(19) United States
(12) Patent Application Publication
(10) Pub. No.: US 2023/0339915 A1
(43) Pub. Date: Oct. 26, 2023

(54) MODULATORS OF ALPHA-1 ANTITRYPSIN

(71) Applicant: Vertex Pharmaceuticals Incorporated, Boston, MA (US)

(72) Inventors: Simon GIROUX, Cambridge, MA (US); Michael Philip CLARK, Concord, MA (US); Qing TANG, Boxborough, MA (US); Philippe Marcel NUHANT, Dorchester, MA (US); Peter JONES, Sharon, MA (US); David MESSERSMITH, Somerville, MA (US); Upul Keerthi BANDARAGE, Lexington, MA (US); Kevin Michael COTTRELL, Cambridge, MA (US); Michael Aaron BRODNEY, Newton, MA (US); Gabrielle Simone FLEMING, Boston, MA (US); Jian WANG, Newton, MA (US); Jinwang XU, Framingham, MA (US); Kevin Brett DANIEL, Boston, MA (US); Michael John BOYD, Sharon, MA (US); Mark A. MORRIS, Somerville, MA (US); Nathan D. WAAL, Somerville, MA (US); Philip Noel COLLIER, Hingham, MA (US); Sarathy KESAVAN, Quincy, MA (US); Steven M. RONKIN, Watertown, MA (US); Hongbo DENG, Wellesley, MA (US); Diane Marie BOUCHER, Beverly, MA (US); Lev T.D. FANNING, San Marcos, CA (US); Amy B. HALL, Wellesley Hills, MA (US); Dennis James HURLEY, San Marcos, CA (US); Mac Arthur JOHNSON, Jr., Derry, NH (US); John Patrick MAXWELL, Hingham, MA (US); Rebecca Jane SWETT, Somerville, MA (US); Timothy Lewis TAPLEY, Cardiff, CA (US); Stephen A. THOMSON, Durham, NC (US); Veronique DAMAGNEZ, Framingham, MA (US)

(21) Appl. No.: 17/916,388

(22) PCT Filed: Apr. 2, 2021
(86) PCT No.: PCT/US2021/025591

§ 371 Date: Sep. 30, 2022

Related U.S. Application Data
(60) Provisional application No. 63/004,713, filed on Apr. 3, 2020.

(51) Int. Cl. C07D 405/04; C07D 209/24; C07D 409/04; C07D 209/22; C07D 405/14; C07D 401/04; C07D 403/04; C07D 413/06; C07D 413/14

(52) U.S. Cl. CPC C07D 405/04; C07D 209/24; C07D 409/04; C07D 209/22; C07D 405/14; C07D 401/04; C07D 403/04; C07D 413/06; C07D 413/14

(57) ABSTRACT

Novel compounds, compositions, and methods of using and preparing the same, which maybe useful for treating alpha-1 antitrypsin deficiency (AATD).

This application claims the benefit of priority of U.S. Provisional Application No. 63/004,713, filed Apr. 3, 2020, the contents of which are incorporated by reference herein in their entirety.

The disclosure provides compounds that are capable of modulating alpha-1 antitrypsin (AAT) activity and methods of treating alpha-1 antitrypsin deficiency (AATD) by administering one or more such compounds.

AATD is a genetic disorder characterized by low circulating levels of AAT. While treatments for AATD exist, there is currently no cure. AAT is produced primarily in liver cells and secreted into the blood, but it is also made by other cell types including lung epithelial cells and certain white blood cells. AAT inhibits several serine proteases secreted by inflammatory cells (most notably neutrophil elastase [NE], proteinase 3, and cathepsin G) and thus protects organs such as the lung from protease-induced damage, especially during periods of inflammation.

The mutation most commonly associated with AATD involves a substitution of lysine for glutamic acid (E342K) in the SERPINA1 gene that encodes the AAT protein. This mutation, known as the Z mutation or the Z allele, leads to misfolding of the translated protein, which is therefore not secreted into the bloodstream and can polymerize within the producing cell. Consequently, circulating AAT levels in individuals homozygous for the Z allele (PiZZ) are markedly reduced; only approximately 15% of mutant Z-AAT protein folds correctly and is secreted by the cell. An additional consequence of the Z mutation is that the secreted Z-AAT has reduced activity compared to wild-type protein, with 40% to 80% of normal antiprotease activity (American thoracic society/European respiratory society, Am J Respir Crit Care Med. 2003; 168(7):818-900; and Ogushi et al. J Clin Invest. 1987; 80(5):1366-74).

The accumulation of polymerized Z-AAT protein within hepatocytes results in a gain-of-function cytotoxicity that can result in cirrhosis or liver cancer later in life and neonatal liver disease in 12% of patients. This accumulation may spontaneously remit but can be fatal in a small number of children. The deficiency of circulating AAT results in unregulated protease activity that degrades lung tissue over time, resulting in emphysema, a form of chronic obstructive pulmonary disease (COPD). This effect is severe in PiZZ individuals and typically manifests in middle age, resulting in a decline in quality of life and shortened lifespan (mean 68 years of age) (Tanash et al. Int J Chron Obstruct Pulm Dis. 2016; 11:1663-9). The effect is more pronounced in PiZZ individuals who smoke, resulting in an even further shortened lifespan (58 years). (Piitulainen and Tanash, COPD 2015; 12(1):36-41). PiZZ individuals account for the majority of those with clinically relevant AATD lung disease. Accordingly, there is a need for additional and effective treatments for AATD.

A milder form of AATD is associated with the SZ genotype in which the Z-allele is combined with an S-allele. The S allele is associated with somewhat reduced levels of circulating AAT but causes no cytotoxicity in liver cells. The result is clinically significant lung disease but not liver disease. (Fregonese and Stolk, Orphanet J Rare Dis. 2008; 33:16.) As with the ZZ genotype, the deficiency of circulating AAT in subjects with the SZ genotype results in unregulated protease activity that degrades lung tissue over time and can result in emphysema, particularly in smokers.

The current standard of care for AAT deficient individuals who have or show signs of developing significant lung or liver disease is augmentation therapy or protein replacement therapy. Augmentation therapy involves administration of a human AAT protein concentrate purified from pooled donor plasma to augment the missing AAT. Although infusions of the plasma protein have been shown to improve survival or slow the rate of emphysema progression, augmentation therapy is often not sufficient under challenging conditions such as during an active lung infection. Similarly, although protein replacement therapy shows promise in delaying progression of disease, augmentation does not restore the normal physiological regulation of AAT in patients and efficacy has been difficult to demonstrate. In addition, augmentation therapy requires weekly visits for treatment and augmentation therapy cannot address liver disease, which is driven by the toxic gain-of-function of the Z allele. Thus, there is a continuing need for new and more effective treatments for AATD.

One aspect of the disclosure provides compounds of Formulae (I), (IIa)-(IIc), (III), (IV), (Va)-(Vc), (VIa)-(VIc), and (VIIa)-(VIIe) as well as tautomers of those compounds, deuterated derivatives of those tautomers and compounds, and pharmaceutically acceptable salts of those compounds, tautomers, or deuterated derivatives that can be employed in the treatment of AATD. For example, compounds of Formula (I), tautomers thereof, deuterated derivatives of those compounds or tautomers, or pharmaceutically acceptable salts of any of the foregoing, can be depicted as:

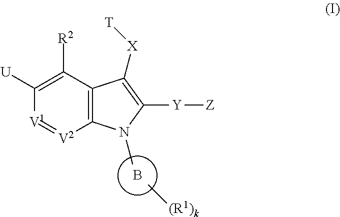

wherein:
V¹ and V² are each independently N or —CR²;
U is —OH or —NH₂;
X is absent or a bond, —(CR^{a}R^{a})ₚ—, or —R^{a′}C═CR^{a′}—;
Y is absent or a bond, —(CR^{b}R^{b})_{q}—, or —R^{b′}C═CR^{b′}—;
T is —CR^{c}R^{c}COOH, —CR^{c}═CR^{c}COOH, —CN, or

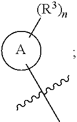

R^{a} and R^{b}, for each occurrence, are each independently hydrogen, halogen, —OH, benzyl, C₁-C₆ alkyl, C₂-C₆ alkenyl, C₁-C₆ haloalkyl, C₁-C₆ alkoxy, or C₁-C₆ haloalkoxy;
R^{a′} and R^{b′}, for each occurrence, are each independently hydrogen, C₁-C₆ alkyl, C₂-C₆ alkenyl, C₁-C₆ haloalkyl, C₁-C₆ alkoxy, or C₁-C₆ haloalkoxy;
R^{c}, for each occurrence, are independently hydrogen, halogen, —OH, benzyl, C₁-C₆ alkyl, C₂-C₆ alkenyl, C₁-C₆ haloalkyl, C₁-C₆ alkoxy, or C₁-C₆ haloalkoxy;
Ring A is C₃-C₁₂ cycloalkyl, 3 to 12-membered heterocyclyl, C₆ or C₁₀ aryl, or 5 to 10-membered heteroaryl;
Z is —CN,

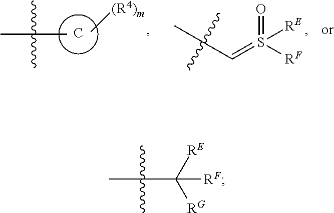

wherein:
when T is not —CN, Ring C is C₃-C₁₂ cycloalkyl, C₆ or C₁₀ aryl, 3 to 12-membered heterocyclyl, or 5 to 10-membered heteroaryl;
when T is —CN, Ring C is C₃-C₁₂ cycloalkyl or 3 to 12-membered heterocyclyl;
R^{E}, R^{F}, and R^{G} are each independently hydrogen, halogen, cyano, C₁-C₆ alkyl, C₂-C₆ alkenyl, C₁-C₆ alkoxy, C₁-C₆ haloalkyl, C₁-C₆ haloalkoxy, —C(═O)R^{s}, —C(═O)OR^{s}, —C(═O)NR^{p}R^{q}, —CR^{p}(═N)OR^{s}, —NR^{p}R^{q}, —NR^{p}C(═O)R^{s}, —NR^{p}C(═O)OR^{s}, —NR^{p}C(═O)NR^{q}R^{r}, —OR^{s}, —OC(═O)R^{s}, or —OC(═O)NR^{p}R^{q}; wherein:
   the C₁-C₆ alkyl or the C₂-C₆ alkenyl of any one of R^{E}, R^{F}, and R^{G} is optionally substituted with 1 to 3 groups selected from cyano, —C(═O)R^{s}, —C(═O)OR^{s}, —C(═O)NR^{p}R^{q}, —NR^{p}C(═O)R^{s}, —NR^{p}C(═O)OR^{s}, —NR^{p}C(═O)NR^{q}R^{r}, —NR^{p}S(═O)ᵣR^{s}, —OR^{s}, —OC(═O)R^{s}, —OC(═O)OR^{s}, —OC(═O)NR^{p}R^{q}, —S(═O)ᵣR^{s}, and —S(═O)ᵣNR^{p}R^{q}; wherein:
      R^{p}, R^{q}, and R^{r}, for each occurrence, are each independently hydrogen, C₁-C₄ alkyl, C₃-C₆ cycloalkyl, or 3 to 6-membered heterocyclyl; wherein:
         the C₁-C₄ alkyl of any one of R^{p}, R^{a}, and R^{r} is optionally substituted with 1 to 3 groups selected from halogen, cyano, —OH, C₁-C₃ alkoxy, —C(═O)NH(C₁-C₂ alkyl), and —C(═O)N(C₁-C₂ alkyl)₂; and
         the C₃-C₆ cycloalkyl or the 3 to 6-membered heterocyclyl of any one of R^{p}, R^{q}, and R^{r} is optionally substituted with 1 to 3 groups selected from halogen, cyano, —OH, C₁-C₃ alkyl, C₁-C₃ alkoxy, C₁-C₃ haloalkyl, C₁-C₃ haloalkoxy, —C(═O)O(C₁-C₂ alkyl), —C(═O)NH(C₁-C₂ alkyl), and —C(═O)N(C₁-C₂ alkyl)₂;
      R^{s}, for each occurrence, is independently hydrogen, C₁-C₄ alkyl, C₃-C₆ cycloalkyl, phenyl, or 5 or 6-membered heteroaryl; wherein:
         the C₁-C₄ alkyl of R^{s} is optionally substituted with 1 to 3 groups selected from halogen, cyano, —OH, —NH₂, —NH(C₁-C₂ alkyl), —N(C₁-C₂ alkyl)₂, C₁-C₃ alkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
         the C₃-C₆ cycloalkyl, the phenyl, or the 5 or 6-membered heteroaryl of R^{s} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
R¹ is halogen, cyano, C₁-C₃ alkyl, C₁-C₃ haloalkyl, C₁-C₃ alkoxy, C₁-C₃ haloalkoxy, or —O—(C₃-C₆ cycloalkyl);
R², for each occurrence, is independently hydrogen, halogen, cyano, C₁-C₆ alkyl, C₂-C₆ alkenyl, C₁-C₆ alkoxy, C₁-C₆ haloalkyl, C₁-C₆ haloalkoxy, C₃-C₆ cycloalkyl, —NR^{h}Rⁱ, phenyl, or 5 or 6-membered heteroaryl; wherein:
   the C₁-C₆ alkyl, the C₂-C₆ alkenyl or the C₃-C₆ cycloalkyl of R² is optionally substituted with 1 to 3 groups selected from cyano, —C(═O)R^{k}, —C(═O)OR^{k}, —C(═O)NR^{h}Rⁱ, —NR^{h}Rⁱ, —NR^{h}C(═O)R^{k}, —NR^{h}C(═O)OR^{k}, —NR^{h}C(═O)NRⁱR^{j}, —NR^{h}S(═O)ₛR^{k}, —OR^{k}, —OC(═O)R^{k}, —OC(═O)OR^{k}, —OC(═O)NR^{h}Rⁱ, —S(═O)ₛR^{k}, and S(═O)ₛNR^{h}Rⁱ; wherein:
      R^{h}, Rⁱ, and R^{j}, for each occurrence, are each independently hydrogen, C₁-C₄ alkyl, or C₃-C₆ cycloalkyl; wherein:
         the C₁-C₄ alkyl of any one of R^{h}, Rⁱ, and R^{j} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
         the C₃-C₆ cycloalkyl of any one of R^{h}, Rⁱ, and R^{j} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
      R^{k}, for each occurrence, is independently hydrogen, C₁-C₄ alkyl, C₃-C₆ cycloalkyl, phenyl, or 5 or 6-membered heteroaryl; wherein:
         —OR^{k} cannot be —OH;
         the C₁-C₄ alkyl of R^{k} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
         the C₃-C₆ cycloalkyl, the phenyl, or the 5 or 6-membered heteroaryl of R^{k} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
R³ and R⁴, for each occurrence, are each independently halogen, cyano, ═O, C₁-C₆ alkyl, C₂-C₆ alkenyl, C₁-C₆ alkoxy, C₁-C₆ haloalkyl, C₁-C₆ haloalkoxy, C₃-C₆ cycloalkyl, —C(═O)R^{y}, —C(═O)OR^{y}, —C(═O)NR^{v}R^{w}, —C(═O)NR^{v}OR^{y}, —C(═O)NR'S(═O)ₜR^{y}, —NR^{v}R^{w}, —NR^{v}C(═O)R^{y}, —NR^{v}C(═O)OR^{y}, —NR^{v}C(═O)NR^{w}R^{x}, —NR^{v}S(═O)ₜR^{y}, —OR^{y}, —OC(═O)R^{y}, —OC(═O)OR^{y}, —OC(═O)NR^{v}R^{w}, —S(═O)ₜR^{y}, —S(═O)ₜNR^{v}R^{w}, —S(═O)ₜNR^{v}C(═O)R^{y}, —P(═O)R^{z}R^{z}, phenyl, or 5 or 6-membered heteroaryl; wherein:
   the C₁-C₆ alkyl, the C₂-C₆ alkenyl, the C₃-C₆ cycloalkyl, the phenyl, or the 5 or 6-membered heteroaryl of any one of R³ and R⁴ is optionally substituted with 1 to 3 groups selected from cyano, —C(═O)R^{y}, —C(═O)OR^{y}, —C(═O)NR^{v}R^{w}, —NR^{v}R^{W}, —NR^{v}C(═O)R^{y}, —NR^{v}C(═O)OR^{y}, —NR^{v}C(═O)NR^{w}R^{x}, —NR^{v}S(═O)ᵣR^{y}, —OR^{y}, —OC(═O)R^{y}, —OC(═O)OR^{y}, —OC(═O)NR^{v}R^{w}, —S(═O)ₜR^{y}, and —S(═O)ₜNR^{v}R^{w}; wherein:
      R^{v}, R^{w}, and R^{x}, for each occurrence, are each independently hydrogen, C₁-C₄ alkyl, C₃-C₆ cycloalkyl, 5 or 6-membered heterocyclyl, or 5 or 6-membered heteroaryl; wherein:
         the C₁-C₄ alkyl of any one of R^{v}, R^{w}, and R^{x} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
         the C₃-C₆ cycloalkyl, the 5 or 6-membered heterocyclyl, or the 5 or 6-membered heteroaryl of any one of R^{v}, R^{w}, and R^{x} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
      R^{y}, for each occurrence, is independently hydrogen, C₁-C₄ alkyl, C₃-C₆ cycloalkyl, phenyl, a 5 or 6-membered heterocyclyl, or a 5 or 6-membered heteroaryl; wherein
         the C₁-C₄ alkyl of R^{y} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
         the C₃-C₆ cycloalkyl, the phenyl, the 5 or 6-membered heterocyclyl, or the 5 or 6-membered heteroaryl of R^{y} is optionally substituted with 1 to 3 groups selected from halogen, cyano, —OH, —NH₂, NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
      R^{z}, for each occurrence, is independently C₁-C₂ alkyl, —OH, or —O(C₁-C₂ alkyl);
k, m, and n are each independently an integer selected from 0, 1, 2, and 3; and
p, q, r, s, and t are each independently an integer selected from 1 and 2.

The compounds of Formulae (I), (IIa)-(IIc), (III), (IV), (Va)-(Vc), (VIa)-(VIc), and (VIIa)-(VIIe) are modulators of AAT activity. In some embodiments, the compounds of Formulae (I), (IIa)-(IIc), (III), (IV), (Va)-(Vc), (VIa)-(VIc), and (VIIa)-(VIIe), as well as tautomers of those compounds, deuterated derivatives of those tautomers and compounds, and pharmaceutically acceptable salts of those compounds, tautomers, or deuterated derivatives have an EC₅₀ of 2.0 μM or less when tested in an AAT Function Assay. In some embodiments, the compounds of Formulae (I), (IIa)-(IIc), (III), (IV), (Va)-(Vc), (VIa)-(VIc), and (VIIa)-(VIIe), as well as tautomers of those compounds, deuterated derivatives of those tautomers and compounds, and pharmaceutically acceptable salts of those compounds, tautomers, or deuterated derivatives have an EC₅₀ of less than 0.5 μM when tested in an AAT Function Assay.

In some embodiments, the compounds of Formulae (I), (IIa)-(IIc), (III), (IV), (Va)-(Vc), (VIa)-(VIc), and (VIIa)-(VIIe), as well as tautomers of those compounds, deuterated derivatives of those tautomers and compounds, and pharmaceutically acceptable salts of those compounds, tautomers, or deuterated derivatives have an IC₅₀ of 5.0 μM or less when tested in a Z-AAT Elastase Activity Assay. In some embodiments, the compounds of Formulae (I), (IIa)-(IIc), (III), (IV), (Va)-(Vc), (VIa)-(VIc), and (VIIa)-(VIIe), as well as tautomers of those compounds, deuterated derivatives of those tautomers and compounds, and pharmaceutically acceptable salts of those compounds, tautomers, or deuterated derivatives have an IC₅₀ of less than 2.0 μM when tested in a Z-AAT Elastase Activity Assay.

In some embodiments, the compounds of Formulae (I), (IIa)-(IIc), (III), (IV), (Va)-(Vc), (VIa)-(VIc), and (VIIa)-(VIIe), as well as tautomers of those compounds, deuterated derivatives of those tautomers and compounds, and pharmaceutically acceptable salts of those compounds, tautomers, or deuterated derivatives have an EC₅₀ of 2.0 μM or less when tested in an AAT Function Assay and have an IC₅₀ of 5.0 μM or less when tested in a Z-AAT Elastase Activity Assay. In some embodiments, the compounds of Formulae (I), (IIa)-(IIc), (III), (IV), (Va)-(Vc), (VIa)-(VIc), and (VIIa)-(VIIe), as well as tautomers of those compounds, deuterated derivatives of those tautomers and compounds, and pharmaceutically acceptable salts of those compounds, tautomers, or deuterated derivatives have an EC₅₀ of less than 0.5 μM when tested in an AAT Function Assay and have an IC₅₀ of 5.0 μM or less when tested in a Z-AAT Elastase Activity Assay. In some embodiments, the compounds of Formulae (I), (IIa)-(IIc), (III), (IV), (Va)-(Vc), (VIa)-(VIc), and (VIIa)-(VIIe), as well as tautomers of those compounds, deuterated derivatives of those tautomers and compounds, and pharmaceutically acceptable salts of those compounds, tautomers, or deuterated derivatives have an EC₅₀ of 2.0 μM or less when tested in an AAT Function Assay and have an IC₅₀ of less than 2.0 μM when tested in a Z-AAT Elastase Activity Assay. In some embodiments, the compounds of Formulae (I), (IIa)-(IIc), (III), (IV), (Va)-(Vc), (VIa)-(VIc), and (VIIa)-(VIIe), as well as tautomers of those compounds, deuterated derivatives of those tautomers and compounds, and pharmaceutically acceptable salts of those compounds, tautomers, or deuterated derivatives have an EC₅₀ of less than 0.5 μM when tested in an AAT Function Assay and have an IC₅₀ of less than 2.0 μM when tested in a Z-AAT Elastase Activity Assay.

In some embodiments, the compounds of Formulae (I), (IIa)-(IIc), (III), (IV), (Va)-(Vc), (VIa)-(VIc), and (VIIa)-(VIIe), as well as tautomers of those compounds, deuterated derivatives of those tautomers and compounds, and pharmaceutically acceptable salts of those compounds, tautomers, or deuterated derivatives are provided for use in the treatment of AATD. In one aspect of the disclosure, the compounds of Formula (I) are selected from Compounds 1-203 and 206-227, tautomers of those compounds, deuterated derivatives of those compounds or tautomers, and pharmaceutically acceptable salts of any of the foregoing for use in the treatment of AATD. In some embodiments of the disclosure, the compounds of the disclosure are selected from Compounds 1-227, tautomers of Compounds 1-227, deuterated derivatives of those compounds or tautomers, and pharmaceutically acceptable salts of any of the foregoing for use in the treatment of AATD.

In some embodiments, the disclosure provides pharmaceutical compositions comprising at least one compound of selected from compounds of Formulae (I), (IIa)-(IIc), (III), (IV), (Va)-(Vc), (VIa)-(VIc), and (VIIa)-(VIIe), tautomers of those compounds, deuterated derivatives of those compounds and tautomers, and pharmaceutically acceptable salts of any of the foregoing. In some embodiments, the pharmaceutical compositions may comprise a compound selected from Compounds 1-227, tautomers of those compounds, deuterated derivatives of those compounds and tautomers, and pharmaceutically acceptable salts of any of the foregoing. These compositions may further include at least one additional active pharmaceutical ingredient and/or at least one carrier.

Another aspect of the disclosure provides methods of treating AATD comprising administering to a subject in need thereof, at least one compound of selected from compounds of Formulae (I), (IIa)-(IIc), (III), (IV), (Va)-(Vc), (VIa)-(VIc), and (VIIa)-(VIIe), tautomers of those compounds, deuterated derivatives of those compounds and tautomers, and pharmaceutically acceptable salts of any of the foregoing or a pharmaceutical composition comprising the at least one such compound, tautomer, deuterated derivative, or pharmaceutically acceptable salt. In some embodiments, the methods comprise administering a compound selected from Compounds 1-227, tautomers of those compounds, deuterated derivatives of those compounds and tautomers, and pharmaceutically acceptable salts of any of the foregoing.

In some embodiments, the methods of treatment include administration of at least one additional active agent to the subject in need thereof, either in the same pharmaceutical composition as the at least one compound of selected from compounds of Formulae (I), (IIa)-(IIc), (III), (IV), (Va)-(Vc), (VIa)-(VIc), and (VIIa)-(VIIe), tautomers of those compounds, deuterated derivatives of those compounds and tautomers, and pharmaceutically acceptable salts of any of the foregoing, or as separate compositions. In some embodiments, the methods comprise administering a compound selected from Compounds 1-227, tautomers of those compounds, deuterated derivatives of those compounds and tautomers, and pharmaceutically acceptable salts of any of the foregoing with at least one additional active agent either in the same pharmaceutical composition or in a separate composition. In some embodiments, the subject in need of treatment carries the ZZ mutation. In some embodiments, the subject in need of treatment carries the SZ mutation.

In some embodiments, the methods of treatment include administration of at least one additional active agent to the subject in need thereof, either in the same pharmaceutical composition as the at least one compound of selected from compounds of Formulae (I), (IIa)-(IIc), (III), (IV), (Va)-(Vc), (VIa)-(VIc), and (VIIa)-(VIIe), tautomers of those compounds, deuterated derivatives of those compounds and tautomers, and pharmaceutically acceptable salts of any of the foregoing, or as separate compositions, wherein the additional active agent is alpha-1 antitrypsin protein (AAT) from the blood plasma of healthy human donors. In some embodiments, the methods comprise administering a compound selected from Compounds 1-227, tautomers of those compounds, deuterated derivatives of those compounds and tautomers, and pharmaceutically acceptable salts of any of the foregoing with at least one additional active agent either in the same pharmaceutical composition or in a separate composition, wherein the additional active agent is alpha-1 antitrypsin protein (AAT) from the blood plasma of healthy human donors.

In some embodiments, the methods of treatment include administration of at least one additional active agent to the subject in need thereof, either in the same pharmaceutical composition as the at least one compound of selected from compounds of Formulae (I), (IIa)-(IIc), (III), (IV), (Va)-(Vc), (VIa)-(VIc), and (VIIa)-(VIIe), tautomers of those compounds, deuterated derivatives of those compounds and tautomers, and pharmaceutically acceptable salts of any of the foregoing, or as separate compositions, wherein the additional active agent is recombinant AAT. In some embodiments, the methods comprise administering a compound selected from Compounds 1-227, tautomers of those compounds, deuterated derivatives of those compounds and tautomers, and pharmaceutically acceptable salts of any of the foregoing with at least one additional active agent either in the same pharmaceutical composition or in a separate composition, wherein the additional active agent is recombinant AAT.

Also provided are methods of modulating AAT, comprising administering to a subject in need thereof, at least one compound of selected from compounds of Formulae (I), (IIa)-(IIc), (III), (IV), (Va)-(Vc), (VIa)-(VIc), and (VIIa)-(VIIe), and tautomers of those compounds, deuterated derivatives of those compounds and tautomers, and pharmaceutically acceptable salts of any of the foregoing or a pharmaceutical composition comprising the at least one compound, tautomer, deuterated derivative, or pharmaceutically acceptable salt. In some embodiments, the methods of modulating AAT comprise administering at least one compound selected from Compounds 1-227, tautomers of those compounds, deuterated derivatives of those compounds and tautomers, and pharmaceutically acceptable salts of any of the foregoing or a pharmaceutical composition comprising the at least one such compound, tautomer, deuterated derivative or pharmaceutically acceptable salt.

Also provided is a compound of Formulae (I), (IIa)-(IIc), (III), (IV), (Va)-(Vc), (VIa)-(VIc), and (VIIa)-(VIIe), and tautomers of those compounds, deuterated derivatives of those compounds and tautomers, and pharmaceutically acceptable salts of any of the foregoing, for use in therapy. In some embodiments, there is provided a compound selected from Compounds 1-227, tautomers of those compounds, deuterated derivatives of those compounds and tautomers, and pharmaceutically acceptable salts of any of the foregoing, for use in therapy.

Also provided is a pharmaceutical composition comprising a compound of Formulae (I), (IIa)-(IIc), (III), (IV), (Va)-(Vc), (VIa)-(VIc), and (VIIa)-(VIIe), and tautomers of those compounds, deuterated derivatives of those compounds and tautomers, and pharmaceutically acceptable salts of any of the foregoing, for use in therapy. In some embodiments, there is provided a pharmaceutical composition comprising a compound selected from Compounds 1-227, tautomers of those compounds, deuterated derivatives of those compounds and tautomers, and pharmaceutically acceptable salts of any of the foregoing, for use in therapy.

### I. DEFINITIONS

The term “AAT” as used herein means alpha-1 antitrypsin or a mutation thereof, including, but not limited to, the AAT gene mutations such as Z mutations. As used herein, “Z-AAT” means AAT mutants which have the Z mutation.

As used herein, “mutations” can refer to mutations in the SERPINA1 gene (the gene encoding AAT) or the effect of alterations in the gene sequence on the AAT protein. A “SERPINA1 gene mutation” refers to a mutation in the SERPINA1 gene, and an “AAT protein mutation” refers to a mutation that results in an alteration in the amino acid sequence of the AAT protein. A genetic defect or mutation, or a change in the nucleotides in a gene in general, results in a mutation in the AAT protein translated from that gene.

As used herein, a patient who is “homozygous” for a particular gene mutation has the same mutation on each allele.

As used herein, a patient who has the PiZZ genotype is a patient who is homozygous for the Z mutation in the AAT protein.

The term “AATD” as used herein means alpha-1 antitrypsin deficiency, which is a genetic disorder characterized by low circulating levels of AAT.

The term “compound,” when referring to a compound of this disclosure, refers to a collection of molecules having an identical chemical structure unless otherwise indicated as a collection of stereoisomers (for example, a collection of racemates, a collection of cis/trans stereoisomers, or a collection of (E) and (Z) stereoisomers), except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this disclosure will depend upon a number of factors including the isotopic purity of reagents used to make the compound and the efficiency of incorporation of isotopes in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

Compounds of the disclosure may optionally be substituted with one or more substituents. It will be appreciated that the phrase “optionally substituted” is used interchangeably with the phrase “substituted or unsubstituted.” In general, the term “substituted,” whether preceded by the term “optionally” or not, refers to the replacement of hydrogen radicals in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by this disclosure are those that result in the formation of stable or chemically feasible compounds.

The term “isotopologue” refers to a species in which the chemical structure differs from a specific compound of this disclosure only in the isotopic composition thereof. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C or ¹⁴C are within the scope of this disclosure.

Unless otherwise indicated, structures depicted herein are also meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the present compounds are within the scope of the disclosure. Unless otherwise stated, all tautomeric forms of the compounds of the disclosure are within the scope of the disclosure.

The term “tautomer,” as used herein, refers to one of two or more isomers of a compound that exist together in equilibrium, and are readily interchanged by migration of an atom or group within the molecule.

“Stereoisomer” refers to both enantiomers and diastereomers.

As used herein, “deuterated derivative” refers to a compound having the same chemical structure as a reference compound, but with one or more hydrogen atoms replaced by a deuterium atom (“D”). It will be recognized that some variation of natural isotopic abundance occurs in a synthesized compound depending on the origin of chemical materials used in the synthesis. The concentration of naturally abundant stable hydrogen isotopes, notwithstanding this variation is small and immaterial as compared to the degree of stable isotopic substitution of deuterated derivatives described herein. Thus, unless otherwise stated, when a reference is made to a “deuterated derivative” of a compound of the disclosure, at least one hydrogen is replaced with deuterium at well above its natural isotopic abundance (which is typically about 0.015%). In some embodiments, the deuterated derivatives of the disclosure have an isotopic enrichment factor for each deuterium atom, of at least 3500 (52.5% deuterium incorporation at each designated deuterium) at least 4500, (67.5% deuterium incorporation), at least 5000 (75% deuterium incorporation) at least 5500 (82.5% deuterium incorporation), at least 6000 (90% deuterium incorporation), at least 6333.3 (95% deuterium incorporation, at least 6466.7 (97% deuterium incorporation, or at least 6600 (99% deuterium incorporation).

The term “isotopic enrichment factor” as used herein means the ratio between the isotopic abundance and the natural abundance of a specified isotope.

The term “alkyl” as used herein, means a straight-chain (i.e., linear or unbranched) or branched, substituted or unsubstituted hydrocarbon chain that is completely saturated or may contain one or more units of saturation, without being fully aromatic. Unless otherwise specified, alkyl groups contain 1-12 alkyl carbon atoms. In some embodiments, alkyl groups contain 1-10 aliphatic carbon atoms. In other embodiments, alkyl groups contain 1-8 aliphatic carbon atoms. In still other embodiments, alkyl groups contain 1-6 alkyl carbon atoms, in other embodiments alkyl groups contain 1-4 alkyl carbon atoms, and in yet other embodiments alkyl groups contain 1-3 alkyl carbon atoms and 1-2 alkyl carbon atoms.

The term “heteroalkyl” as used herein, refers to aliphatic groups wherein one or two carbon atoms are independently replaced by one or more of oxygen, sulfur, nitrogen, phosphorus, or silicon. Heteroalkyl groups may be substituted or unsubstituted, branched or unbranched.

The term “alkenyl” as used herein, means a straight-chain (i.e., linear or unbranched), branched, substituted or unsubstituted hydrocarbon chain that contains one or more carbon-to-carbon double bonds.

The terms “cycloalkyl,” “cyclic alkyl,” “carbocyclyl,” and “carbocycle” refer to a fused, spirocyclic, or bridged monocyclic C₃₋₉ hydrocarbon or a fused, spirocyclic, or bridged bicyclic or tricyclic, C₈₋₁₄ hydrocarbon that is completely saturated or that contains one or more units of unsaturation, but which is not fully aromatic, wherein any individual ring in said bicyclic ring system has 3-9 members. Typically, a cycloalkyl is completely saturated, while a carbocyclyl may contain one or more units of unsaturation but is not aromatic. In some embodiments, the cycloalkyl or carbocycle group contains 3 to 12 carbon atoms. In some embodiments, the cycloalkyl or carbocycle group contains 3 to 8 carbon atoms. In some embodiments, the cycloalkyl or carbocycle group contains 3 to 6 carbon atoms.

The term “heterocycle,” “heterocyclyl,” or “heterocyclic” as used herein refers to fused, spirocyclic, or bridged non-aromatic, monocyclic, bicyclic, or tricyclic ring systems in which one or more ring members is a heteroatom. In some embodiments, “heterocycle,” “heterocyclyl,” or “heterocyclic” group has 3 to 14 ring members in which one or more ring members is a heteroatom independently selected from oxygen, sulfur, nitrogen, phosphorus, and silicon and each ring in the system contains 3 to 9 ring members. In some embodiments, the heterocyclyl contains 3 to 12 ring member atoms. In some embodiments, the heterocyclyl contains 3 to 8 ring member atoms. In some embodiments, the heterocyclyl contains 3 to 6 ring member atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “alkoxy” as used herein, refers to an alkyl group, as previously defined, wherein one carbon of the alkyl group is replaced by an oxygen (“alkoxy”) atom, respectively, provided that the oxygen atom is linked between two carbon atoms. A “cyclic alkoxy” refers to a monocyclic, fused, spirocyclic, bicyclic, bridged bicyclic, tricyclic, or bridged tricyclic hydrocarbon that contains at least one alkoxy group, but is not aromatic. Non-limiting examples of cyclic alkoxy groups include tetrahydropyranyl, tetrahydrofuranyl, oxetanyl, 8-oxabicyclo[3.2.1]octanyl, and oxepanyl.

The terms “haloalkyl” and “haloalkoxy” means an alkyl or alkoxy, as the case may be, which is substituted with one or more halogen atoms. The term “halogen” or means F, Cl, Br, or I. In some embodiments, the halogen is selected from F, Cl, and Br. Examples of haloalkyls include —CHF₂, —CH₂F, —CF₃, —CF₂—, or perhaloalkyl, such as, —CF₂CF₃.

As used herein, “═O” refers to an oxo group.

As used herein, a “cyano” or “nitrile” groups refers to —C≡N.

As used herein, a “hydroxy” group refers to —OH.

As used herein, “aromatic groups” or “aromatic rings” refer to chemical groups that contain conjugated, planar ring systems with delocalized pi electron orbitals comprised of [4n+2] p orbital electrons, wherein n is an integer ranging from 0 to 6. Nonlimiting examples of aromatic groups include aryl and heteroaryl groups.

The term “aryl” refers to monocyclic, bicyclic, and tricyclic ring systems having a total of 5 to 14 ring members, wherein at least one ring in the system is aromatic and wherein each ring in the system contains 3 to 7 ring members. In some embodiments, an aryl contains 6 or 10 carbon atoms. A nonlimiting example of an aryl group is a phenyl ring.

The term “heteroaryl” refers to monocyclic, bicyclic, and tricyclic ring systems having a total of 5 to 10 ring members, wherein at least one ring in the system is aromatic, at least one ring in the system contains one or more heteroatoms, and wherein each ring in the system contains 3 to 7 ring members. In some embodiments, a heteroaryl contains 6 or 10 ring atoms.

Examples of useful protecting groups for nitrogen-containing groups, such as amine groups, include, for example, t-butyl carbamate (Boc), benzyl (Bn), tetrahydropyranyl (THP), 9-fluorenylmethyl carbamate (Fmoc) benzyl carbamate (Cbz), acetamide, trifluoroacetamide, triphenylmethylamine, benzylideneamine, and p-toluenesulfonamide (OTs). Methods of adding (a process generally referred to as “protecting”) and removing (process generally referred to as “deprotecting”) such amine protecting groups are well-known in the art and available, for example, in P. J. Kocienski, Protecting Groups, Thieme, 1994, which is hereby incorporated by reference in its entirety and in Greene and Wuts, Protective Groups in Organic Synthesis, 3rd Edition (John Wiley & Sons, New York, 1999).

Examples of suitable solvents that may be used in this disclosure include, but not limited to, water, methanol (MeOH), ethanol (EtOH), dichloromethane or “methylene chloride” (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Examples of suitable bases that may be used in this disclosure include, but not limited to, 1,8-diazabicyclo[5.4.0]undec-7-ene (DBU), potassium tert-butoxide (KOtBu), potassium carbonate (K₂CO₃), N-methylmorpholine (NN), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH) and sodium methoxide (NaOMe; NaOCH₃).

The disclosure includes pharmaceutically acceptable salts of the disclosed compounds. A salt of a compound of is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this disclosure. Suitable pharmaceutically acceptable salts are, for example, those disclosed in S. M. Berge, et al. J. Pharmaceutical Sciences, 1977, 66, 1-19.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

The terms “patient” and “subject” are used interchangeably and refer to an animal including a human.

The terms “effective dose,” “effective amount,” “therapeutically effective dose,” and “therapeutically effective amount” are used interchangeably herein and refer to that amount of a compound that produces the desired effect for which it is administered (e.g., improvement in AATD or a symptom of AATD, lessening the severity of AATD or a symptom of AATD, and/or reducing the rate of onset or incidence of AATD or a symptom of AATD). The exact amount of an effective dose will depend on the purpose of the treatment, and will be ascertainable by one skilled in the art using known techniques (see, e.g., Lloyd (1999) The Art, Science and Technology of Pharmaceutical Compounding).

As used herein, the term “treatment and its cognates (e.g., “treat,” “treating”) refer to improving AATD or its symptoms in a subject, delaying the onset of AATD or its symptoms in a subject, or lessening the severity of AATD or its symptoms in a subject. “Treatment” and its cognates as used herein, include, but are not limited to the following: improved liver and/or spleen function, lessened jaundice, improved lung function, lessened lung diseases and/or pulmonary exacerbations (e.g., emphysema), lessened skin disease (e.g., necrotizing panniculitis), increased growth in children, improved appetite, and reduced fatigue. Improvements in or lessening the severity of any of these symptoms can be readily assessed according to methods and techniques known in the art or subsequently developed.

The terms “about” and “approximately”, when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent. Typically, the term “about” refers to a variation of up to 10%, up to 5%, or up to 2% of a stated value.

Any one or more of the compounds of Formulae (I), (IIa)-(IIc), (III), (IV), (Va)-(Vc), (VIa)-(VIc), and (VIIa)-(VIIe), tautomers of those compounds, deuterated derivatives of those compounds or tautomers, and pharmaceutically acceptable salts of any of the foregoing may be administered once daily, twice daily, or three times daily for the treatment of AATD. In some embodiments, the any one or more compounds are selected from Compounds 1-227, tautomers of those compounds, deuterated derivatives of those compounds or tautomers, and pharmaceutically acceptable salts of any of the foregoing. In some embodiments, at least one compound chosen from compounds of Formulae (I), (IIa)-(IIc), (III), (IV), (Va)-(Vc), (VIa)-(VIc), and (VIIa)-(VIIe), tautomers of those compounds, deuterated derivatives of those compounds or tautomers, and pharmaceutically acceptable salts of any of the foregoing is administered once daily. In some embodiments, a compound selected from Compounds 1-227, tautomers of those compounds, deuterated derivatives of those compounds or tautomers, and pharmaceutically acceptable salts of any of the foregoing is administered once daily. In some embodiments, at least one compound selected from compounds of Formulae (I), (IIa)-(IIc), (III), (IV), (Va)-(Vc), (VIa)-(VIc), and (VIIa)-(VIIe), tautomers of those compounds, deuterated derivatives of those compounds or tautomers, and pharmaceutically acceptable salts of any of the foregoing are administered twice daily. In some embodiments, a compound selected from Compounds 1-227, tautomers of those compounds, deuterated derivatives of those compounds or tautomers, and pharmaceutically acceptable salts of any of the foregoing is administered twice daily. In some embodiments, at least one compound chosen from compounds of Formulae (I), (IIa)-(IIc), (III), (IV), (Va)-(Vc), (VIa)-(VIc), and (VIIa)-(VIIe), tautomers of those compounds, deuterated derivatives of those compounds or tautomers, and pharmaceutically acceptable salts of any of the foregoing are administered three times daily. In some embodiments, a compound selected from Compounds 1-227, tautomers of those compounds, deuterated derivatives of those compounds or tautomers, and pharmaceutically acceptable salts of any of the foregoing is administered three times daily.

Any one or more of the compounds of (I), (IIa)-(IIc), (III), (IV), (Va)-(Vc), (VIa)-(VIc), and (VIIa)-(VIIe), tautomers of those compounds, deuterated derivatives of those compounds or tautomers, and pharmaceutically acceptable salts of any of the foregoing may be administered in combination with AAT augmentation therapy or AAT replacement therapy for the treatment of AATD. In some embodiments, the any one or more compounds are selected from Compounds 1-227, tautomers of those compounds, deuterated derivatives of those compounds or tautomers, and pharmaceutically acceptable salts of any of the foregoing.

As used herein, “AAT augmentation therapy” refers to the use of alpha-1 antitrypsin protein (AAT) from the blood plasma of healthy human donors to augment (increase) the alpha-1 antitrypsin levels circulating in the blood. “AAT replacement therapy” refers to administration of recombinant AAT.

In some embodiments, 10 mg to 1,500 mg, 100 mg to 1,800 mg, 100 mg to 500 mg, 200 mg to 600 mg, 200 mg to 800 mg, 400 mg to 2,000 mg, 400 mg to 2,500 mg or 400 mg to 600 mg of a compound of Formulae (I), (IIa)-(IIc), (III), (IV), (Va)-(Vc), (VIa)-(VIc), and (VIIa)-(VIIe), tautomers of those compounds, deuterated derivatives of those compounds or tautomers, and pharmaceutically acceptable salts of any of the foregoing is administered once daily, twice daily, or three times daily. In some embodiments, 10 mg to 1,500 mg, 100 mg to 1,800 mg, 100 mg to 500 mg, 200 mg to 600 mg, 200 mg to 800 mg, 400 mg to 2,000 mg, or 400 mg to 600 mg of a compound selected from Compounds 1-227, tautomers of those compounds, deuterated derivatives of those compounds or tautomers, and pharmaceutically acceptable salts of any of the foregoing is administered once daily, twice daily, or three times daily. In some embodiments, 10 mg to 1,500 mg, 100 mg to 1,800 mg, 100 mg to 500 mg, 200 mg to 600 mg, 200 mg to 800 mg, 400 mg to 2,000 mg, or 400 mg to 600 mg of a compound selected from Compounds 1-227, is administered once daily, twice daily, or three times daily.

One of ordinary skill in the art would recognize that, when an amount of a compound is disclosed, the relevant amount of a pharmaceutically acceptable salt form of the compound is an amount equivalent to the concentration of the free base of the compound. It is noted that the disclosed amounts of the compounds, tautomers, deuterated derivatives, and pharmaceutically acceptable salts are based upon the free base form of the reference compound. For example, “10 mg of at least one compound chosen from compounds of Formula (I) and pharmaceutically acceptable salts thereof” includes 10 mg of a compound of Formula (I) and a concentration of a pharmaceutically acceptable salt of compounds of Formula (I) equivalent to 10 mg of compounds of Formula (I).

As used herein, the term “ambient conditions” means room temperature, open air condition and uncontrolled humidity condition.

It should be understood that references herein to methods of treatment (e.g., methods of treating AATD) using one or more compounds (e.g., compounds of Formulae (I), (IIa)-(IIc), (III), (IV), (Va)-(Vc), (VIa)-(VIc), and (VIIa)-(VIIe)), as well as tautomers of those compounds, deuterated derivatives of those compounds and tautomers, and pharmaceutically acceptable salts of those compounds) should also be interpreted as references to:
one or more compounds (e.g., compounds of Formulae (I), (IIa)-(IIc), (III), (IV), (Va)-(Vc), (VIa)-(VIc), and (VIIa)-(VIIe)), as well as tautomers of those compounds, deuterated derivatives of those compounds and tautomers, and pharmaceutically acceptable salts of those compounds) for use in methods of treating, e.g., AATD; and/or
the use of one or more compounds (e.g., compounds of Formulae (I), (IIa)-(IIc), (III), (IV), (Va)-(Vc), (VIa)-(VIc), and (VIIa)-(VIIe)), as well as tautomers of those compounds, deuterated derivatives of those compounds and tautomers, and pharmaceutically acceptable salts of those compounds) in the manufacture of a medicament for treating, e.g., AATD.

### EXAMPLE EMBODIMENTS

Without limitation, some embodiments of the disclosure include:
1. A compound represented by the following structural formula:

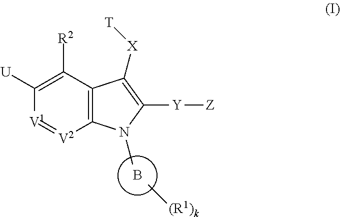

a tautomer thereof, a deuterated derivative of the compound or tautomer, or a pharmaceutically acceptable salt of any of the foregoing, wherein:
   V¹ and V² are each independently N or —CR²;
   U is —OH or —NH₂;
   X is absent or a bond, —(CR^{a}R^{a})ₚ—, or —R^{a′}C═CR^{a′}—;
   Y is absent or a bond, —(CR^{b}R^{b})_{q}—, or —R^{b′}C═CR^{b′}—;
   T is —CR^{c}R^{c}COOH, —CR^{c}═CR^{c}COOH, —CN, or

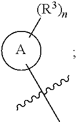

R^{a} and R^{b}, for each occurrence, are each independently hydrogen, halogen, —OH, benzyl, C₁-C₆ alkyl, C₂-C₆ alkenyl, C₁-C₆ haloalkyl, C₁-C₆ alkoxy, or C₁-C₆ haloalkoxy;
R^{a′} and R^{b′}, for each occurrence, are each independently hydrogen, C₁-C₆ alkyl, C₂-C₆ alkenyl, C₁-C₆ haloalkyl, C₁-C₆ alkoxy, or C₁-C₆ haloalkoxy;
R^{c}, for each occurrence, are independently hydrogen, halogen, —OH, benzyl, C₁-C₆ alkyl, C₂-C₆ alkenyl, C₁-C₆ haloalkyl, C₁-C₆ alkoxy, or C₁-C₆ haloalkoxy;
Ring A is C₃-C₁₂ cycloalkyl, 3 to 12-membered heterocyclyl, C₆ or C₁₀ aryl, or 5 to 10-membered heteroaryl;
Ring B is C₄-C₁₂ cycloalkyl, C₆ or C₁₀ aryl, benzyl, or 5 to 10-membered heteroaryl;
Z is —CN,

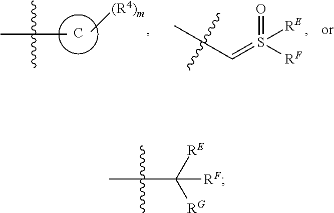

wherein:
   when T is not —CN, Ring C is C₃-C₁₂ cycloalkyl, C₆ or C₁₀ aryl, 3 to 12-membered heterocyclyl, or 5 to 10-membered heteroaryl;
   when T is —CN, Ring C is C₃-C₁₂ cycloalkyl or 3 to 12-membered heterocyclyl;
   R^{E}, R^{F}, and R^{G} are each independently hydrogen, halogen, cyano, C₁-C₆ alkyl, C₂-C₆ alkenyl, C₁-C₆ alkoxy, C₁-C₆ haloalkyl, C₁-C₆ haloalkoxy, —C(═O)R^{s}, —C(═O)OR^{s}, —C(═O)NR^{p}R^{q}, —CR^{p}(═N)OR^{s}, —NR^{p}R^{q}, —NR^{p}C(═O)R^{s}, —NR^{p}C(═O)OR^{s}, —NR^{p}C(═O)NR^{q}R^{r}, —OR^{s}, —OC(═O)R^{s}, or —OC(═O)NR^{p}R^{q}; wherein:
      the C₁-C₆ alkyl or the C₂-C₆ alkenyl of any one of R^{E}, R^{F}, and R^{G} is optionally substituted with 1 to 3 groups selected from cyano, —C(═O)R^{s}, —C(═O)OR^{s}, —C(═O)NR^{p}R^{q}, —NR^{p}C(═O)R^{s}, —NR^{p}C(═O)OR^{s}, —NR^{p}C(═O)NR^{q}R^{r}, —NR^{p}S(═O)ᵣR^{s}, —OR^{s}, —OC(═O)R^{s}, —OC(═O)OR^{s}, —OC(═O)NR^{p}R^{q}, —S(═O)ᵣR^{s}, and —S(═O)ᵣNR^{p}R^{q}; wherein:
         R^{p}, R^{q}, and R^{r}, for each occurrence, are each independently hydrogen, C₁-C₄ alkyl, C₃-C₆ cycloalkyl, or 3 to 6-membered heterocyclyl; wherein:
         the C₁-C₄ alkyl of any one of R^{p}, R^{a}, and R^{r} is optionally substituted with 1 to 3 groups selected from halogen, cyano, —OH, C₁-C₃ alkoxy, —C(═O)NH(C₁-C₂ alkyl), and —C(═O)N(C₁-C₂ alkyl)₂; and
         the C₃-C₆ cycloalkyl or the 3 to 6-membered heterocyclyl of any one of R^{p}, R^{q}, and R^{r} is optionally substituted with 1 to 3 groups selected from halogen, cyano, —OH, C₁-C₃ alkyl, C₁-C₃ alkoxy, C₁-C₃ haloalkyl, C₁-C₃ haloalkoxy, —C(═O)O(C₁-C₂ alkyl), —C(═O)NH(C₁-C₂ alkyl), and —C(═O)N(C₁-C₂ alkyl)₂;
         R^{s}, for each occurrence, is independently hydrogen, C₁-C₄ alkyl, C₃-C₆ cycloalkyl, phenyl, or 5 or 6-membered heteroaryl; wherein:
         the C₁-C₄ alkyl of R^{s} is optionally substituted with 1 to 3 groups selected from halogen, cyano, —OH, —NH₂, —NH(C₁-C₂ alkyl), —N(C₁-C₂ alkyl)₂, C₁-C₃ alkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
         the C₃-C₆ cycloalkyl, the phenyl. or the 5 or 6-membered heteroaryl of R^{s} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
   R¹ is halogen, cyano, C₁-C₃ alkyl, C₁-C₃ haloalkyl, C₁-C₃ alkoxy, C₁-C₃ haloalkoxy, or —O—(C₃-C₆ cycloalkyl);
   R², for each occurrence, is independently hydrogen, halogen, cyano, C₁-C₆ alkyl, C₂-C₆ alkenyl, C₁-C₆ alkoxy, C₁-C₆ haloalkyl, C₁-C₆ haloalkoxy, C₃-C₆ cycloalkyl, —NR^{h}Rⁱ, phenyl, or 5 or 6-membered heteroaryl; wherein:
      the C₁-C₆ alkyl, the C₂-C₆ alkenyl or the C₃-C₆ cycloalkyl of R² is optionally substituted with 1 to 3 groups selected from cyano, —C(═O)R^{k}, —C(═O)OR^{k}, —C(═O)NR^{h}Rⁱ, —NR^{h}Rⁱ, —NR^{h}C(═O)R^{k}, —NR^{h}C(═O)OR^{k}, —NR^{h}C(═O)NRⁱR^{j}, —NR^{h}S(═O)ₛR^{k}, —OR^{k}, —OC(═O)R^{h}, —OC(═O)OR^{h}, —OC(═O)NR^{h}Rⁱ, —S(═O)ₛR^{k}, and S(═O)ₛNR^{h}Rⁱ; wherein:
         R^{h}, Rⁱ, and R^{j}, for each occurrence, are each independently hydrogen, C₁-C₄ alkyl, or C₃-C₆ cycloalkyl; wherein:
         the C₁-C₄ alkyl of any one of R^{h}, Rⁱ, and R^{j} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
         the C₃-C₆ cycloalkyl of any one of R^{h}, Rⁱ, and R^{j} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
         R^{k}, for each occurrence, is independently hydrogen, C₁-C₄ alkyl, C₃-C₆ cycloalkyl, phenyl, or 5 or 6-membered heteroaryl; wherein:
         —OR^{k} cannot be —OH;
         the C₁-C₄ alkyl of R^{k} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
         the C₃-C₆ cycloalkyl, the phenyl, or the 5 or 6-membered heteroaryl of R^{k} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
   R³ and R⁴, for each occurrence, are each independently halogen, cyano, ═O, C₁-C₆ alkyl, C₂-C₆ alkenyl, C₁-C₆ alkoxy, C₁-C₆ haloalkyl, C₁-C₆ haloalkoxy, C₃-C₆ cycloalkyl, —C(═O)R^{y}, —C(═O)OR^{y}, —C(═O)NR^{v}R^{w}, —C(═O)NR^{v}OR^{y}, -(═O)NR^{v}S(═O)ₜR^{y}, —NR^{v}R^{w}, —NR^{v}C(═O)R^{y}, —NR^{v}C(═O)OR^{y}, —NR^{v}C(═O)NR^{w}R^{x}, —NR'S(═O)ₜR^{y}, —OR^{y}, —OC(═O)R^{y}, —OC(═O)OR^{Y}, —OC(═O)NR^{v}R^{w}, —S(═O)ₜR^{y}, —S(═O)ₜNR^{v}R^{w}, —S(═O)ₜNR^{v}C(═O)R^{y}, —P(═O)R^{z}R^{z}, phenyl, or 5 or 6-membered heteroaryl; wherein:
      the C₁-C₆ alkyl, the C₂-C₆ alkenyl, the C₃-C₆ cycloalkyl, or the 5 or 6-membered heteroaryl of any one of R³ and R⁴ is optionally substituted with 1 to 3 groups selected from cyano, —C(═O)R^{y}, —C(═O)OR^{y}, —C(═O)NR^{v}R^{w}, —NR^{v}R^{W}, —NR^{v}C(═O)R^{y}, —NR^{v}C(═O)OR^{y}, —NR^{v}C(═O)NR^{w}R^{x}, —NR^{v}S(═O)ᵣR^{y}, —OR^{y}, —OC(═O)R^{y}, —OC(═O)OR^{y}, —OC(═O)NR^{v}R^{w}, —S(═O)ₜR^{y}, and —S(═O)ₜNR^{v}R^{w}; wherein:
         R^{v}, R^{w}, and R^{x}, for each occurrence, are each independently hydrogen, C₁-C₄ alkyl, C₃-C₆ cycloalkyl, 5 or 6-membered heterocyclyl, or 5 or 6-membered heteroaryl; wherein:
         the C₁-C₄ alkyl of any one of R^{v}, R^{w}, and R^{x} is optionally substituted with 1 to 3 groups selected from halogen, cyano —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
         the C₃-C₆ cycloalkyl, the 5 or 6-membered heterocyclyl, or the 5 or 6-membered heteroaryl of any one of R^{v}, R^{w}, and R^{x} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
         R^{y}, for each occurrence, is independently hydrogen, C₁-C₄ alkyl, C₃-C₆ cycloalkyl, phenyl, a 5 or 6-membered heterocyclyl, or a 5 or 6-membered heteroaryl; wherein
         the C₁-C₄ alkyl of R^{y} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
         the C₃-C₆ cycloalkyl, the phenyl, the 5 or 6-membered heterocyclyl, or the 5 or 6-membered heteroaryl of R^{y} is optionally substituted with 1 to 3 groups selected from halogen, cyano, —OH, —NH₂, NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
         R^{z}, for each occurrence, is independently C₁-C₂ alkyl, —OH, or —O(C₁-C₂ alkyl);
   k, n, and o are each independently an integer selected from 0, 1, 2, and 3; and
   p, q, r, s, and t are each independently an integer selected from 1 and 2.
2. The compound, tautomer, deuterated derivative, or pharmaceutically acceptable salt according Embodiment 1, represented by Formula (IIa):

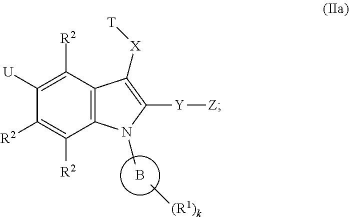

wherein:
   Y is absent or a bond, —CR^{b}R^{b}—, or —R^{b′}C═CR^{b′}—;
   R^{b}, for each occurrence, is independently hydrogen or C₁-C₂ alkyl;
   Ring B is optionally substituted with R¹ and Ring B is C₄-C₆ cycloalkyl, phenyl or 5 or 6-membered heteroaryl;
and wherein all other variables not specifically defined herein are as defined in Embodiment 1.
3. The compound, tautomer, deuterated derivative, or pharmaceutically acceptable salt, according to Embodiment 1 represented by Formulae (IIb) or (IIc):

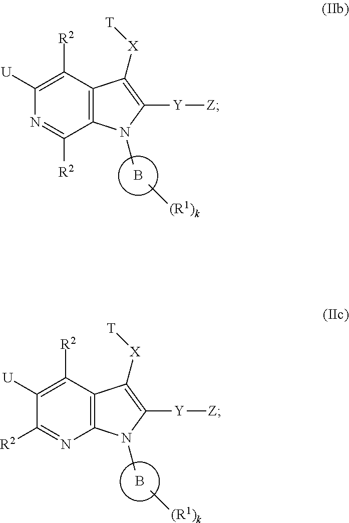

wherein:
   Y is absent or a bond, —CR^{b}R^{b}—, or —R^{b′}C═CR^{b′}—;
   R^{b}, for each occurrence, is independently hydrogen or C₁-C₂ alkyl;
Ring B is optionally substituted with R¹ and Ring B is C₄-C₆ cycloalkyl, phenyl or 5 or 6-membered heteroaryl;
and wherein all other variables not specifically defined herein are as defined in Embodiment 1.
4. The compound, tautomer, deuterated derivative, or pharmaceutically acceptable salt according to any one of Embodiments 1 to 3, wherein Y is absent or a bond, —CH₂—, or —HC═CH—; and wherein all other variables not specifically defined herein are as defined in any one of the preceding Embodiments.
5. The compound, tautomer, deuterated derivative, or pharmaceutically acceptable salt according to any one of Embodiments 1 to 4, represented by Formula (III):

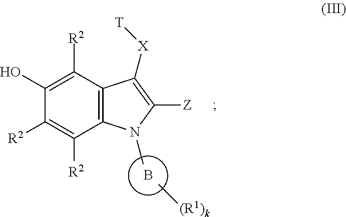

wherein:
   X is absent or a bond, or —(CR^{a}R^{a})ₚ—;
   R^{a}, for each occurrence, is each independently hydrogen or C₁-C₂ alkyl;
   R^{c}, for each occurrence, is independently hydrogen, F, —OH, benzyl, C₁-C₂ alkyl, or C₁-C₂ alkoxy;
   Ring B is optionally substituted with R¹ and Ring B is cyclobutyl, phenyl, pyridinyl, or pyrimidinyl;
and wherein all other variables not specifically defined herein are as defined in any one of the preceding Embodiments.
6. The compound, tautomer, deuterated derivative, or pharmaceutically acceptable salt according to any one of Embodiments 1 to 5, wherein:
   X is absent or a bond, —CH₂—, —CHCH₃—, —CH₂CH₂—, or —CHCH₃CH₂—;
   Ring B is optionally substituted with Rⁱ and Ring B is cyclobutyl, phenyl, pyridine-4-yl, or pyrimidin-4-yl;
and wherein all other variables not specifically defined herein are as defined in any one of the preceding Embodiments.
7. The compound, tautomer, deuterated derivative, or pharmaceutically acceptable salt according to any one of Embodiments 1 to 6, represented by Formula (IV):

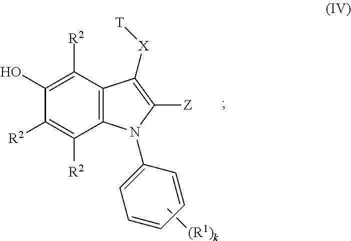

wherein:
   T is —CH₂COOH, —CHCH₃COOH, —CHC₂H₅COOH, —C(CH₃)₂COOH, —CF₂COOH, —CH═CHCOOH, —C(CH₃)(OH)COOH, —C(CH₃)(OCH₃)COOH, cyano, —CH(benzyl)COOH, or Ring A optionally substituted with R³;
   when Z is Ring C, Ring C is optionally substituted with R⁴ and Ring C is C₃-C₆ cycloalkyl, 4 to 8-membered heterocyclyl, phenyl, or 5 or 6-membered heteroaryl; and
   R¹ is halogen, C₁-C₂ alkyl, or C₁-C₂ haloalkyl; and
   k is an integer selected from 0, 1 and 2;
and wherein all other variables not specifically defined herein are as defined in any one of the preceding Embodiments.
8. The compound, tautomer, deuterated derivative, or pharmaceutically acceptable salt according to any one of Embodiments 1 to 7, wherein R¹ is F, Cl, or —CH₃; and wherein all other variables not specifically defined herein are as defined in any one of preceding Embodiments.
9. The compound, tautomer, deuterated derivative, or pharmaceutically acceptable salt according to any one of Embodiments 1 to 8, wherein when T is Ring A, Ring A is optionally substituted with R³, and Ring A is C₃-C₇ cycloalkyl, 4 to 6-membered heterocyclyl, phenyl, or 5 or 6-membered heteroaryl; and
wherein all other variables not specifically defined herein are as defined in any one of preceding Embodiments.
10. The compound, tautomer, deuterated derivative, or pharmaceutically acceptable salt according to any one of Embodiments 1 to 9, wherein when T is Ring A, Ring A is optionally substituted with R³, and Ring A is C₃-C₇ cycloalkyl, 4 to 6-membered heterocyclyl, phenyl, or 5 or 6-membered heteroaryl containing one or two nitrogen atoms; and wherein all other variables not specifically defined herein are as defined in any one of preceding Embodiments.
11. The compound, tautomer, deuterated derivative, or pharmaceutically acceptable salt according to any one of Embodiments 1 to 10, wherein when T is Ring A, Ring A is optionally substituted with R³, and Ring A is selected from

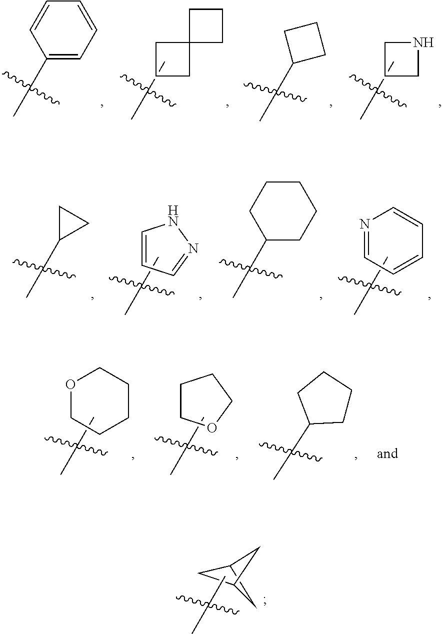

and wherein all other variables not specifically defined herein are as defined in any one of preceding Embodiments.
12. The compound, tautomer, deuterated derivative, or pharmaceutically acceptable salt according to any one of Embodiments 1 to 11, wherein when T is Ring A, Ring A is optionally substituted with R³, and Ring A is selected from

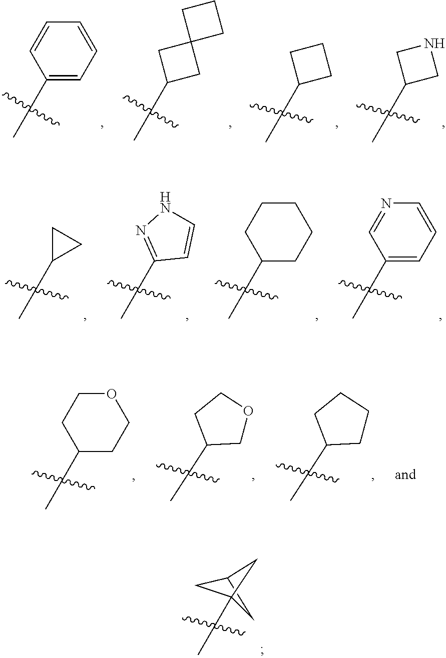

and wherein all other variables not specifically defined herein are as defined in any one of preceding Embodiments.
13. The compound, tautomer, deuterated derivative, or pharmaceutically acceptable salt according to any one of Embodiments 1 to 12, wherein when Z is Ring C, Ring C is optionally substituted with R⁴, and Ring C is C₃-C₄ cycloalkyl or 4 to 6-membered heterocyclyl; and wherein all other variables not specifically defined herein are as defined in any one of the preceding Embodiments.
14. The compound, tautomer, deuterated derivative, or pharmaceutically acceptable salt according to any one of Embodiments 1 to 13, wherein when Z is Ring C, Ring C is optionally substituted with R⁴, and Ring C is

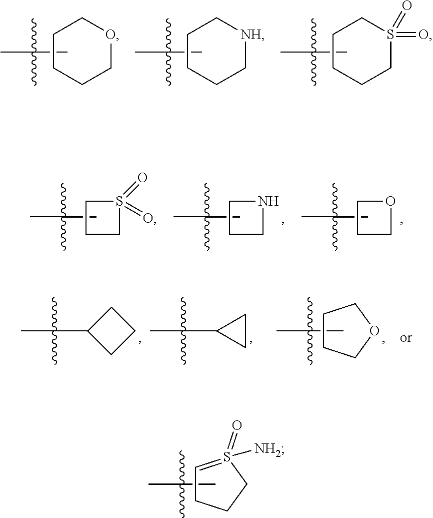

and wherein all other variables not specifically defined herein are as defined in any one of the preceding Embodiments.
15. The compound, tautomer, deuterated derivative, or pharmaceutically acceptable salt according to any one of Embodiments 1 to 14, wherein when Z is Ring C, Ring C is optionally substituted with R⁴, and Ring C is

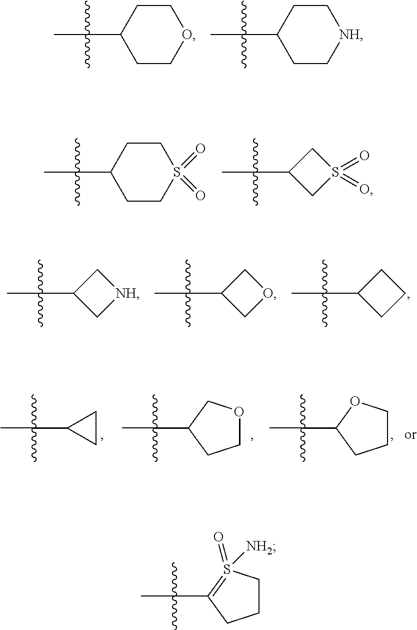

and wherein all other variables not specifically defined herein are as defined in any one of the preceding Embodiments.
16. The compound, tautomer, deuterated derivative, or pharmaceutically acceptable salt according to any one of Embodiments 1 to 12, wherein when Z is

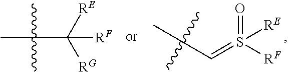

R^{E}, R^{F}, and R^{G} are each independently hydrogen, halogen, cyano, C₁-C₆ alkyl, C₁-C₆ alkoxy, C₁-C₆ haloalkyl, —C(═O)OR^{s}, —C(═O)NR^{p}R^{q}, —CR^{p}(═N)OR^{s}, —NR^{p}R^{q}, or —OR^{s}; wherein:
the C₁-C₆ alkyl of any one of R^{E}, R^{F}, and R^{G} is optionally substituted with 1 to 3 groups selected from cyano and —OR^{s}; wherein:
   R^{p} and R^{a}, for each occurrence, are each independently hydrogen or C₁-C₄ alkyl; and
   R^{s}, for each occurrence, is independently hydrogen or C₁-C₄ alkyl;
and wherein all other variables not specifically defined herein are as defined in any one of preceding Embodiments.
17. The compound, tautomer, deuterated derivative, or pharmaceutically acceptable salt according to any one of Embodiments 1 to 12 and 16, wherein when Z is

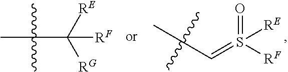

R^{E}, R^{F}, and R^{G} are each independently hydrogen, halogen, C₁-C₂ alkyl, —NR^{p}R^{q}, or —OR^{s}; wherein:
the C₁-C₂ alkyl of any one of R^{E}, R^{F}, and R^{G} is optionally substituted with 1 to 3 groups selected from cyano, —OH, and —OCH₃; wherein:
   R^{p} and R^{a}, for each occurrence, are each independently hydrogen or C₁-C₂ alkyl; and
   R⁸, for each occurrence, is independently hydrogen or C₁-C₂ alkyl;
and wherein all other variables not specifically defined herein are as defined in any one of preceding Embodiments.
18. The compound, tautomer, deuterated derivative, or pharmaceutically acceptable salt according to any one of Embodiments 1 to 12, 16, and 17, wherein:
   when Z is

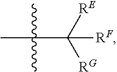

R^{E}, R^{F}, and R^{G} are each independently hydrogen, F, —CH₂CN, —OH, —OCH₃, —CH₃, —C₂H₅, or —CH₂OCH₃; and
   when Z is

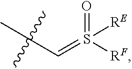

R^{E} and R^{F} are each independently —CH₃ or —NH₂;
and wherein all other variables not specifically defined herein are as defined in any one of preceding Embodiments.
19. The compound, tautomer, deuterated derivative, or pharmaceutically acceptable salt according to any one of Embodiments 1 to 18, represented by Formulae (Va), (Vb), or (Vc):

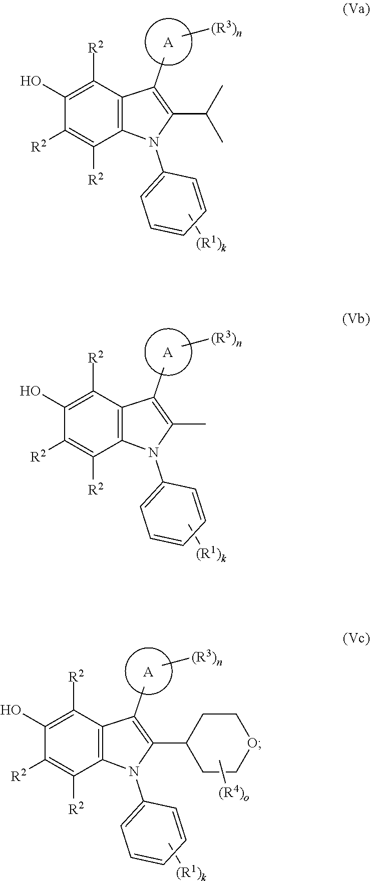

wherein all other variables not specifically defined herein are as defined in any one of preceding Embodiments.
20. The compound, tautomer, deuterated derivative, or pharmaceutically acceptable salt according to any one of Embodiments 1 to 19, represented by Formulae (VIa), (VIb), or (VIc):

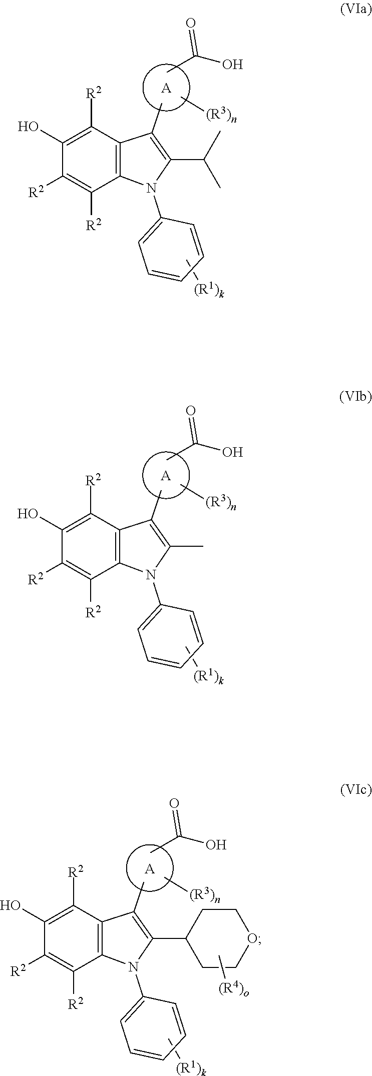

wherein n is an integer selected from 0, 1, and 2; and wherein all other variables not specifically defined herein are as defined in any one of preceding Embodiments.
21. The compound, tautomer, deuterated derivative, or pharmaceutically acceptable salt according to any one of Embodiments 1 to 20, represented by Formulae (VIIa), (VIIb), (VIIc), (VIId), or (VIIe):

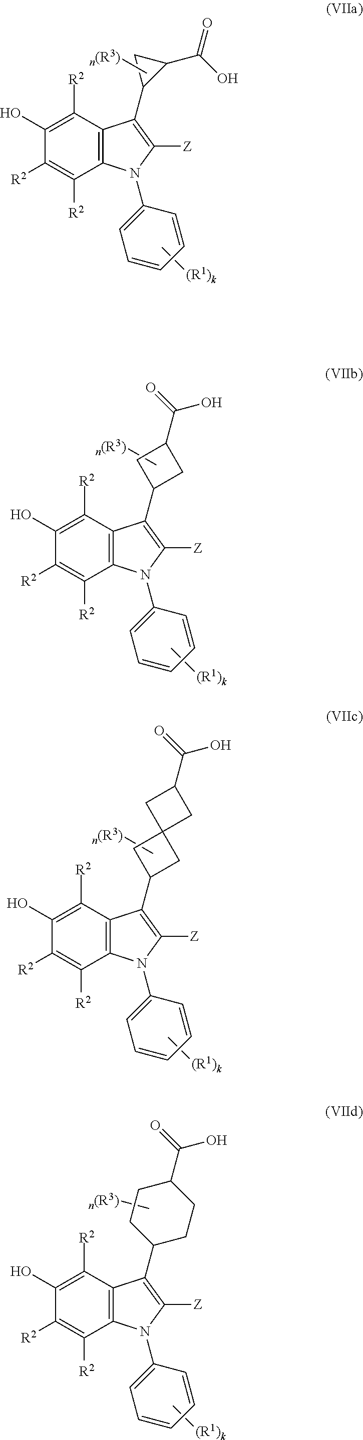

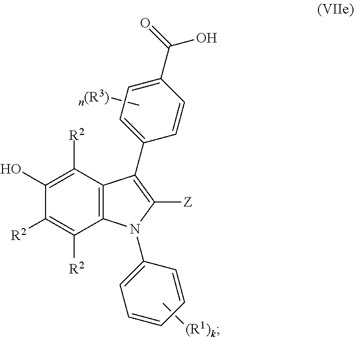

wherein n is an integer selected from 0, 1, and 2; and wherein all other variables not specifically defined herein are as defined in any one of preceding Embodiments.
22. The compound, tautomer, deuterated derivative, or pharmaceutically acceptable salt according to any one of Embodiments 1 to 21, wherein R², for each occurrence, is independently hydrogen, halogen, cyano, C₁-C₄ alkyl, C₁-C₄ alkoxy, C₁-C₄ haloalkyl, —NR^{h}Rⁱ, or cyclopropyl; wherein R^{h} and Rⁱ, for each occurrence, is independently hydrogen or C₁-C₄ alkyl; and wherein all other variables not specifically defined herein are as defined in any one of preceding Embodiments.
23. The compound, tautomer, deuterated derivative, or pharmaceutically acceptable salt according to any one of Embodiments 1 to 22, wherein R², for each occurrence, is independently hydrogen, F, Cl, —CH₃, —NH₂, or cyclopropyl; and wherein all other variables not specifically defined herein are as defined in any one of preceding Embodiments.
24. The compound, tautomer, deuterated derivative, or pharmaceutically acceptable salt according to any one of Embodiments 1 to 23, wherein R³, for each occurrence, is independently halogen, cyano, ═O, C₁-C₆ alkyl, C₁-C₆ alkoxy, C₁-C₆ haloalkyl, —C(═O)OR^{y}, —C(═O)NR^{v}S(═O)₂R^{y}, —S(═O)₂NR^{v}R^{w}, —S(═O)₂NR^{v}C(═O)R^{w}, —P(═O)R^{z}R^{z}, or 5 or 6-membered heteroaryl; wherein:
   the C₁-C₆ alkyl or the 5-membered heteroaryl of R³ is optionally substituted with 1 to 3 groups selected from cyano, —C(═O)OR^{y}, —OR^{y}, and —NR^{v}R^{w}; wherein:
      R^{v} and R^{w}, for each occurrence, are each independently hydrogen or C₁-C₄ alkyl; and
      R^{y}, for each occurrence, is independently hydrogen or C₁-C₄ alkyl;
and wherein all other variables not specifically defined herein are as defined in any one of preceding Embodiments.
25. The compound, tautomer, deuterated derivative, or pharmaceutically acceptable salt according to any one of Embodiments 1 to 24, wherein R³, for each occurrence, is independently halogen, cyano, ═O, C₁-C₄ alkyl, C₁-C₄ alkoxy, C₁-C₄ haloalkyl, —C(═O)OR^{y}, —C(═O)NR^{v}S(═O)₂R^{y}, —S(═O)₂NR^{v}R^{w}, —S(═O)₂NR^{v}C(═O)R^{y}, or 5-membered heteroaryl; wherein:
   the C₁-C₄ alkyl or the 5-membered heteroaryl of R³ is optionally substituted with 1 to 3 groups selected from cyano, —C(═O)OR^{y}, —OR^{y}, and —NR^{v}R^{w}; wherein:
      R^{v} and R^{w}, for each occurrence, are each independently hydrogen or C₁-C₂ alkyl; and
      R^{y}, for each occurrence, is independently hydrogen or C₁-C₂ alkyl;
and wherein all other variables not specifically defined herein are as defined in any one of preceding Embodiments.
26. The compound, tautomer, deuterated derivative, or pharmaceutically acceptable salt according to any one of Embodiments 1 to 25, wherein R³, for each occurrence, is independently halogen, cyano, ═O, C₁-C₂ alkyl, C₁-C₂ alkoxy, C₁-C₂ haloalkyl, —C(═O)OR^{y}, —C(═O)NR'S(═O)₂R^{y}, —S(═O)₂NR^{v}R^{w}, —S(═O)₂NR^{v}C(═O)R^{y}, tetrazolyl, or oxadiazolyl; wherein:
   the C₁-C₂ alkyl or the oxadiazolyl of R³ is optionally substituted with 1 to 3 groups selected from cyano, —COOH, and —OH; wherein:
      R^{v} and R^{w}, for each occurrence, are each independently hydrogen or —CH₃; and
      R^{y}, for each occurrence, is independently hydrogen or —CH₃;
and wherein all other variables not specifically defined herein are as defined in any one of preceding Embodiments.
27. The compound, tautomer, deuterated derivative, or pharmaceutically acceptable salt according to any one of Embodiments 1 to 26, wherein R³, for each occurrence, is independently F, cyano, ═O, —CH₃, —CH₂F, —CHF₂, —CF₃, —CH₂OH, —CH₂OCH₃, —OCH₃, —COOH, —CH₂COOH, —C(═O)NHS(═O)₂CH₃, —S(═O)₂NHCH₃, —S(═O)₂NHC(═O)CH₃, tetrazol-5-yl, 1,2,4-oxadiazol-5(4H)-onyl, or 1,3,4-oxadiazol-2(3H)-onyl; and wherein all other variables not specifically defined herein are as defined in any one of preceding Embodiments.
28. The compound, tautomer, deuterated derivative, or pharmaceutically acceptable salt according to any one of Embodiments 1 to 27, wherein R⁴, for each occurrence, is independently halogen, cyano, C₁-C₆ alkyl, C₁-C₆ haloalkyl, —C(═O)R^{y}, —C(═O)OR^{y}, C(═O)NR^{v}R^{w}, —NR^{v}R^{w}, —OR^{y}, or —P(═O)R^{z}R^{z}; wherein:
   R^{v} and R^{w}, for each occurrence, are each independently hydrogen or C₁-C₄ alkyl; and
   R^{y}, for each occurrence, is independently hydrogen or C₁-C₄ alkyl;
and wherein all other variables not specifically defined herein are as defined in any one of preceding Embodiments.
29. The compound, tautomer, deuterated derivative, or pharmaceutically acceptable salt according to any one of Embodiments 1 to 28, wherein R⁴, for each occurrence, is independently halogen, cyano, C₁-C₄ alkyl, C₁-C₄ haloalkyl, —C(═O)R^{y}, —C(═O)OR^{y}, C(═O)NR^{v}R^{w}, —NR^{v}R^{w}, or —OR^{y}; wherein:
   R^{v} and R^{w}, for each occurrence, are each independently hydrogen or C₁-C₂ alkyl; and
   R^{y}, for each occurrence, is independently hydrogen or C₁-C₄ alkyl;
and wherein all other variables not specifically defined herein are as defined in any one of preceding Embodiments.
30. The compound, tautomer, deuterated derivative, or pharmaceutically acceptable salt according to any one of Embodiments 1 to 29, wherein R⁴, for each occurrence, is independently halogen, cyano, C₁-C₂ alkyl, C₁-C₂ haloalkyl, —C(═O)OR^{y}, or —OR^{y}; wherein:
   R^{y}, for each occurrence, is independently hydrogen or C₁-C₄ alkyl; and wherein all other variables not specifically defined herein are as defined in any one of preceding Embodiments.
31. The compound, tautomer, deuterated derivative, or pharmaceutically acceptable salt according to any one of Embodiments 1 to 30, wherein R⁴, for each occurrence, is —C(═O)OC(CH₃)₃; and wherein all other variables not specifically defined herein are as defined in any one of preceding Embodiments.
32. The compound, tautomer, deuterated derivative, or pharmaceutically acceptable salt according to any one of Embodiments 1 to 31, wherein m is 0; and wherein all other variables not specifically defined herein are as defined in any one of preceding Embodiments.
33. A compound selected from Compounds 1-227, a tautomer thereof, a deuterated derivative of the compound or tautomer, or a pharmaceutically acceptable salt of any of the foregoing.
34. A pharmaceutical composition comprising at least one compound, tautomer, deuterated derivative, or pharmaceutically acceptable salt according to any one of Embodiments 1 to 33.
35. A method of treating alpha-1 antitrypsin (AAT) deficiency comprising administering to a patient in need thereof a therapeutically effective amount of at least one compound, tautomer, deuterated derivative, or pharmaceutically acceptable salt according to any one of Embodiments 1 to 33, or a therapeutically effective amount of a pharmaceutical composition according to Embodiment 34.
36. A method of modulating alpha-1 antitrypsin (AAT) activity comprising the step of contacting said AAT with a therapeutically effective amount of at least one compound, tautomer, deuterated derivative, or pharmaceutically acceptable salt according to any one of Embodiments 1 to 33, or a therapeutically effective amount of a pharmaceutical composition according to Embodiment 34.
37. The method of Embodiment 35 or Embodiment 36, wherein said therapeutically effective amount of the at least one compound, tautomer, deuterated derivative, or pharmaceutically acceptable salt is administered in combination with AAT augmentation therapy and/or AAT replacement therapy.

### II. COMPOUNDS AND COMPOSITIONS

In some embodiments, a compound of the disclosure is a compound of Formula (I):

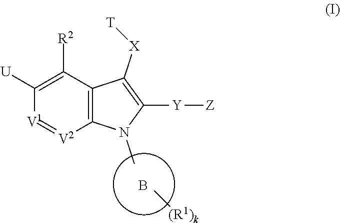

a tautomer thereof, a deuterated derivative of that compound or tautomer, or a pharmaceutically acceptable salt of any of the foregoing, wherein:
V¹ and V² are each independently N or —CR²;
U is —OH or —NH₂;
X is absent or a bond, —(CR^{a}R^{a})ₚ—, or —R^{a′}C═CR^{a′}—;
Y is absent or a bond, —(CR^{b}R^{b})_{q}—, or —R^{b′}C═CR^{b′}—;
T is —CR^{c}R^{c}COOH, —CR^{c}═CR^{c}COOH, —CN, or

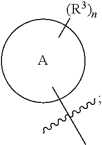

R^{a} and R^{b}, for each occurrence, are each independently hydrogen, halogen, —OH, benzyl, C₁-C₆ alkyl, C₂-C₆ alkenyl, C₁-C₆ haloalkyl, C₁-C₆ alkoxy, or C₁-C₆ haloalkoxy;
R^{a′} and R^{b′}, for each occurrence, are each independently hydrogen, C₁-C₆ alkyl, C₂-C₆ alkenyl, C₁-C₆ haloalkyl, C₁-C₆ alkoxy, or C₁-C₆ haloalkoxy;
R^{c}, for each occurrence, are independently hydrogen, halogen, —OH, benzyl, C₁-C₆ alkyl, C₂-C₆ alkenyl, C₁-C₆ haloalkyl, C₁-C₆ alkoxy, or C₁-C₆ haloalkoxy;
Ring A is C₃-C₁₂ cycloalkyl, 3 to 12-membered heterocyclyl, C₆ or C₁₀ aryl, or 5 to 10-membered heteroaryl;
Ring B is C₄-C₁₂ cycloalkyl, C₆ or C₁₀ aryl, benzyl, or 5 to 10-membered heteroaryl;
Z is —CN,

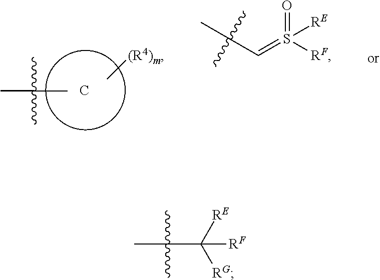

wherein:
when T is not —CN, Ring C is C₃-C₁₂ cycloalkyl, C₆ or C₁₀ aryl, 3 to 12-membered heterocyclyl, or 5 to 10-membered heteroaryl;
when T is —CN, Ring C is C₃-C₁₂ cycloalkyl or 3 to 12-membered heterocyclyl;
R^{E}, R^{F}, and R^{G} are each independently hydrogen, halogen, cyano, C₁-C₆ alkyl, C₂-C₆ alkenyl, C₁-C₆ alkoxy, C₁-C₆ haloalkyl, C₁-C₆ haloalkoxy, —C(═O)R^{s}, —C(═O)OR^{s}, —C(═O)NR^{p}R^{q}, —CR^{p}(═N)OR^{s}, —NR^{p}R^{q}, —NR^{p}C(═O)R^{s}, —NR^{p}C(═O)OR^{s}, —NR^{p}C(═O)NR^{q}R^{r}, —OR^{s}, —OC(═O)R^{s}, or —OC(═O)NR^{p}R^{q}; wherein:
   the C₁-C₆ alkyl or the C₂-C₆ alkenyl of any one of R^{E}, R^{F}, and R^{G} is optionally substituted with 1 to 3 groups selected from cyano, —C(═O)R^{s}, —C(═O)OR^{s}, —C(═O)NR^{p}R^{q}, —NR^{p}C(═O)R^{s}, —NR^{p}C(═O)OR^{s}, —NR^{p}C(═O)NR^{q}R^{r}, —NR^{p}S(═O)ᵣR^{s}, —OR^{s}, —OC(═O)R^{s}, —OC(═O)OR^{s}, —OC(═O)NR^{p}R^{q}, —S(═O)ᵣR^{s}, and —S(═O)ᵣNR^{p}R^{q};
wherein:
   R^{p}, R^{a}, and R^{r}, for each occurrence, are each independently hydrogen, C₁-C₄ alkyl, C₃-C₆ cycloalkyl, or 3 to 6-membered heterocyclyl; wherein:
      the C₁-C₄ alkyl of any one of R^{p}, R^{a}, and R^{r} is optionally substituted with 1 to 3 groups selected from halogen, cyano, —OH, C₁-C₃ alkoxy, —C(═O)NH(C₁-C₂ alkyl), and —C(═O)N(C₁-C₂ alkyl)₂; and
      the C₃-C₆ cycloalkyl or the 3 to 6-membered heterocyclyl of any one of R^{p}, R^{q}, and R^{r} is optionally substituted with 1 to 3 groups selected from halogen, cyano, —OH, C₁-C₃ alkyl, C₁-C₃ alkoxy, C₁-C₃ haloalkyl, C₁-C₃ haloalkoxy, —C(═O)O(C₁-C₂ alkyl), —C(═O)NH(C₁-C₂ alkyl), and —C(═O)N(C₁-C₂ alkyl)₂;
   R^{s}, for each occurrence, is independently hydrogen, C₁-C₄ alkyl, C₃-C₆ cycloalkyl, phenyl, or 5 or 6-membered heteroaryl; wherein:
      the C₁-C₄ alkyl of R^{s} is optionally substituted with 1 to 3 groups selected from halogen, cyano, —OH, —NH₂, —NH(C₁-C₂ alkyl), —N(C₁-C₂ alkyl)₂, C₁-C₃ alkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
      the C₃-C₆ cycloalkyl, the phenyl, or the 5 or 6-membered heteroaryl of R^{s} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
R¹ is halogen, —CN, C₁-C₃ alkyl, C₁-C₃ haloalkyl, C₁-C₃ alkoxy, C₁-C₃ haloalkoxy, or —O—(C₃-C₆ cycloalkyl);
R², for each occurrence, is independently hydrogen, halogen, cyano, C₁-C₆ alkyl, C₂-C₆ alkenyl, C₁-C₆ alkoxy, C₁-C₆ haloalkyl, C₁-C₆ haloalkoxy, C₃-C₆ cycloalkyl, —NR^{h}Rⁱ, phenyl, or 5 or 6-membered heteroaryl; wherein:
   the C₁-C₆ alkyl, the C₂-C₆ alkenyl or the C₃-C₆ cycloalkyl of R² is optionally substituted with 1 to 3 groups selected from cyano, —C(═O)R^{k}, —C(═O)OR^{k}, —C(═O)NR^{h}Rⁱ, —NR^{h}Rⁱ, —NR^{h}C(═O)R^{k}, —NR^{h}C(═O)OR^{k}, —NR^{h}C(═O)NRⁱR^{j}, —NR^{h}S(═O)ₛR^{k}, —OR^{k}, —OC(═O)R^{h}, —OC(═O)OR^{k}, —OC(═O)NR^{h}Rⁱ, —S(═O)ₛR^{k}, and S(═O)ₛNR^{h}Rⁱ; wherein:
      R^{h}, Rⁱ, and R^{j}, for each occurrence, are each independently hydrogen, C₁-C₄ alkyl, or C₃-C₆ cycloalkyl; wherein:
         the C₁-C₄ alkyl of any one of R^{h}, Rⁱ, and R^{j} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
         the C₃-C₆ cycloalkyl of any one of R^{h}, Rⁱ, and R^{j} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
      R^{k}, for each occurrence, is independently hydrogen, C₁-C₄ alkyl, C₃-C₆ cycloalkyl, phenyl, or 5 or 6-membered heteroaryl; wherein:
         —OR^{k} cannot be —OH;
         the C₁-C₄ alkyl of R^{k} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
         the C₃-C₆ cycloalkyl, the phenyl, or the 5 or 6-membered heteroaryl of R^{k} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
R³ and R⁴, for each occurrence, are each independently halogen, cyano, ═O, C₁-C₆ alkyl, C₂-C₆ alkenyl, C₁-C₆ alkoxy, C₁-C₆ haloalkyl, C₁-C₆ haloalkoxy, C₃-C₆ cycloalkyl, —C(═O)R^{y}, —C(═O)OR^{y}, —C(═O)NR^{v}R^{w}, —C(═O)NR^{v}OR^{Y}, —C(═O)NR^{v}S(═O)ₜR^{y}, —NR^{v}R^{w}, —NR^{v}C(═O)R^{y}, —NR^{v}C(═O)OR^{y}, —NR^{v}C(═O)NR^{w}R^{x}, —NR'S(═O)ₜR^{y}, —OR^{y}, —OC(═O)R^{y}, —OC(═O)OR^{y}, —OC(═O)NR^{v}R^{w}, —S(═O)ₜR^{y}, —S(═O)ₜNR^{v}R^{w}, —S(═O)ₜNR^{v}C(═O)R^{y}, —P(═O)R^{z}R^{z}, phenyl, or a 5 or 6-membered heteroaryl; wherein:
   the C₁-C₆ alkyl, the C₂-C₆ alkenyl, or the C₃-C₆ cycloalkyl of any one of R³ and R⁴ is optionally substituted with 1 to 3 groups selected from cyano, —C(═O)R^{y}, —C(═O)OR^{y}, —C(═O)NR^{v}R^{w}, —NR^{v}R^{W}, —NR^{v}C(═O)R^{y}, —NR^{v}C(═O)OR^{y}, —NR^{v}C(═O)NR^{w}R^{x}, —NR^{v}S(═O)ᵣR^{y}, —OR^{y}, —OC(═O)R^{y}, —OC(═O)OR^{y}, —OC(═O)NR^{v}R^{w}, —S(═O)ₜR^{y}, and —S(═O)ₜNR^{v}R^{w}; wherein:
      R^{v}, R^{w}, and R^{x}, for each occurrence, are each independently hydrogen, C₁-C₄ alkyl, C₃-C₆ cycloalkyl, 5 or 6-membered heterocyclyl, or 5 or 6-membered heteroaryl; wherein:
         the C₁-C₄ alkyl of any one of R^{v}, R^{w}, and R^{x} is optionally substituted with 1 to 3 groups selected from halogen, cyano —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
         the C₃-C₆ cycloalkyl, the 5 or 6-membered heterocyclyl, or the 5 or 6-membered heteroaryl of any one of R^{v}, R^{w}, and R^{x} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
      R^{y}, for each occurrence, is independently hydrogen, C₁-C₄ alkyl, C₃-C₆ cycloalkyl, phenyl, a 5 or 6-membered heterocyclyl, or a 5 or 6-membered heteroaryl; wherein
         the C₁-C₄ alkyl of R^{y} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
         the C₃-C₆ cycloalkyl, the phenyl, the 5 or 6-membered heterocyclyl, or the 5 or 6-membered heteroaryl of R^{y} is optionally substituted with 1 to 3 groups selected from halogen, cyano, —OH, —NH₂, NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
      R^{z}, for each occurrence, is independently C₁-C₂ alkyl, —OH, or —O(C₁-C₂ alkyl);
k, m, and n are each independently an integer selected from 0, 1, 2, and 3; and
p, q, r, s, and t are each independently an integer selected from 1 and 2.

In some embodiments, the compound, tautomer, deuterated derivative, or pharmaceutically acceptable salt of the disclosure is represented by Formula (IIa):

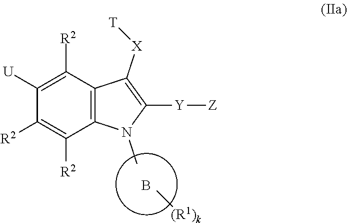

wherein:
Y is absent or a bond, —CR^{b}R^{b}—, or —R^{b′}C═CR^{b}—;
R^{b}, for each occurrence, is independently hydrogen or C₁-C₂ alkyl;
Ring B is optionally substituted with Rⁱ and Ring B is C₄-C₆ cycloalkyl, phenyl, or 5 or 6-membered heteroaryl;
   and wherein all other variables are as defined for Formula (I).

In some embodiments, the compound, tautomer, deuterated derivative, or pharmaceutically acceptable salt of the disclosure is represented by Formula (IIb) or Formulae (IIc):

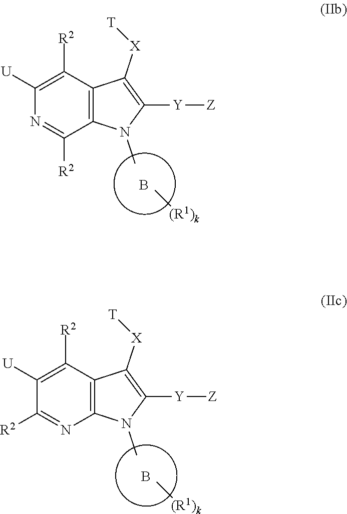

wherein:
Y is absent or a bond, —CR^{b}R^{b}—, or —R^{b′}C═CR^{b′}—;
R^{b}, for each occurrence, is independently hydrogen or C₁-C₂ alkyl;
Ring B is optionally substituted with Rⁱ and Ring B is C₄-C₆ cycloalkyl, phenyl, or 5 or 6-membered heteroaryl;
   and wherein all other variables are as defined for Formula (I).

In some embodiments, Y is absent or a bond, or is selected from —CH₂— and —HC═CH— in the compound, tautomer, deuterated derivative, or pharmaceutically acceptable salt of the Formula (I), (IIa), (IIb), or (IIc), and all other variables not specifically defined herein are as defined in any one of the preceding embodiments.

In some embodiments, the compound, tautomer, deuterated derivative, or pharmaceutically acceptable salt of the disclosure is represented by Formula (III):

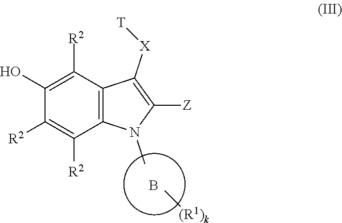

wherein:
X is absent or a bond, or —(CR^{a}R^{a})ₚ—;
R^{a}, for each occurrence, is each independently hydrogen or C₁-C₂ alkyl;
R^{c}, for each occurrence, is independently hydrogen, F, —OH, benzyl, C₁-C₂ alkyl, or C₁-C₂ alkoxy;
Ring B is optionally substituted with Rⁱ and Ring B is cyclobutyl, phenyl, pyridinyl, or pyrimidinyl;
   and wherein all other variables not specifically defined herein are as defined in any one of the preceding embodiments.

In some embodiments, X is absent or a bond, or is selected from —CH₂—, —CHCH₃—, —CH₂CH₂—, and —CHCH₃CH₂— in the compound, tautomer, deuterated derivative, or pharmaceutically acceptable salt of any one of Formulae (I), (IIa), (IIb), (IIc) or (III); Ring B is optionally substituted with Rⁱ and Ring B is selected from cyclobutyl, phenyl, pyridin-4-yl, and pyrimidin-4-yl; and wherein all other variables not specifically defined herein are as defined in any one of the preceding embodiments.

In some embodiments, the compound, tautomer, deuterated derivative, or pharmaceutically acceptable salt of the disclosure is represented by Formula (IV):

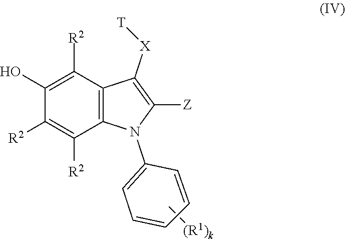

wherein:
T is —CH₂COOH, —CHCH₃COOH, —CHC₂H₅COOH, —C(CH₃)₂COOH, —CF₂COOH, —CH═CHCOOH, —C(CH₃)(OH)COOH, —C(CH₃)(OCH₃)COOH, —CN, —CH(benzyl)COOH, or Ring A optionally substituted with R³;
when Z is Ring C, Ring C is optionally substituted with R⁴ and Ring C is C₃-C₆ cycloalkyl, 4 to 8-membered heterocyclyl, phenyl, or 5 or 6-membered heteroaryl; and
R¹ is halogen, C₁-C₂ alkyl, or C₁-C₂ haloalkyl; and
k is an integer selected from 0, 1 and 2;
   and wherein all other variables not specifically defined herein are as defined in any one of the preceding embodiments.

In some embodiments, R¹ is F, Cl, or —CH₃ in the compound, tautomer, deuterated derivative, or pharmaceutically acceptable salt of any one of Formula (I), (IIa), (IIb), (IIc), (III), or (IV); and all other variables not specifically defined herein are as defined in any one of the preceding embodiments.

In some embodiments, T is Ring A in the compound, tautomer, deuterated derivative, or pharmaceutically acceptable salt of any one of Formula (I), (IIa), (IIb), (IIc), (III), or (IV), Ring A is optionally substituted with R³ and Ring A is C₃-C₇ cycloalkyl, 4 to 6-membered heterocyclyl, phenyl, or 5 or 6-membered heteroaryl; and all other variables are as defined in any one of the preceding embodiments.

In some embodiments, Ring A is optionally substituted with R³ and Ring A is C₃-C₇ cycloalkyl, 5 or 6-membered heterocyclyl, phenyl, or 4 to 6-membered heteroaryl containing one or two nitrogen atoms; and all other variables not specifically defined herein are as defined in any one of the preceding embodiments.

In some embodiments of Formula (I), (IIa), (IIb), (IIc), (III), or (IV), T is Ring A optionally substituted with R³, and Ring A is selected from:

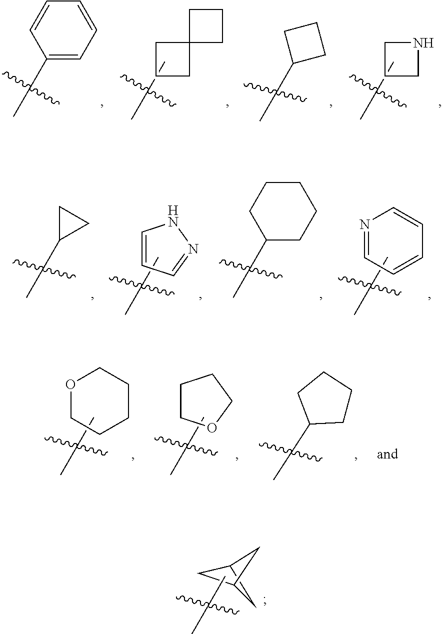

and all other variables not specifically defined herein are as defined in any one of the preceding embodiments.

In some embodiments of Formula (I), (IIa), (IIb), (IIc), (III), or (IV), T is Ring A optionally substituted with R³, and Ring A is selected from:

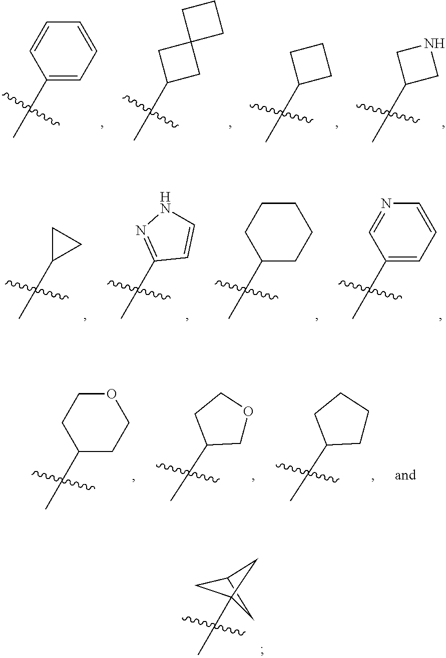

and all other variables not specifically defined herein are as defined in any one of the preceding embodiments.

In some embodiments of Formula (I), (IIa), (IIb), (IIc), (III), or (IV), Z is Ring C optionally substituted with R⁴, and Ring C is a 3 or 4-membered cycloalkyl or a 4 to 6-membered heterocyclyl; and all other variables not specifically defined herein are as defined in any one of the preceding embodiments.

In some embodiments of Formula (I), (IIa), (IIb), (IIc), (III), or (IV), Z is Ring C optionally substituted with R⁴, and Ring C is selected from:

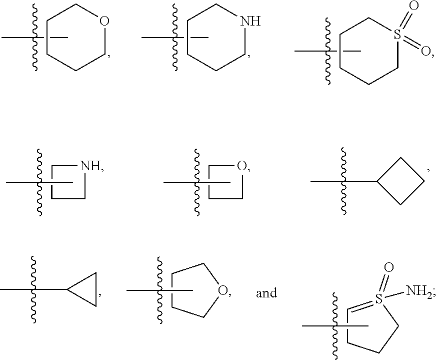

and all other variables not specifically defined herein are as defined in any one of the preceding embodiments.

In some embodiments of Formula (I), (IIa), (IIb), (IIc), (III), or (IV), Z is Ring C is optionally substituted with R⁴ and Ring C is selected from:

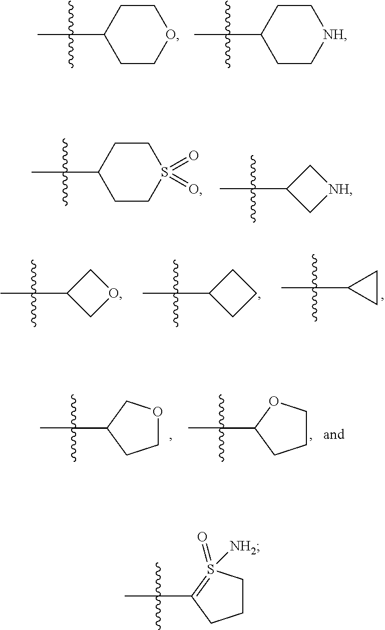

and all other variables not specifically defined herein are as defined in any one of the preceding embodiments.

In some embodiments of Formula (I), (IIa), (IIb), (IIc), (III), or (IV), Z is

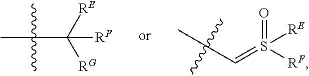

R^{E}, R^{F}, and R^{G} are each independently hydrogen, halogen, cyano, C₁-C₆ alkyl, C₁-C₆ alkoxy, C₁-C₆ haloalkyl, —C(═O)OR^{s}, —C(═O)NR^{p}R^{q}, —CR^{p}(═N)OR^{s}, —NR^{p}R^{q}, or —OR^{s}; wherein:
the C₁-C₆ alkyl of any one of R^{E}, R^{F}, and R^{G} is optionally substituted with 1 to 3 groups selected from cyano and —OR^{s}; wherein:
   R^{p} and R^{a}, for each occurrence, are each independently hydrogen or C₁-C₄ alkyl; and
   R^{s}, for each occurrence, is independently hydrogen or C₁-C₄ alkyl;
      and wherein all other variables not specifically defined herein are as defined in any one of the preceding embodiments.

In some embodiments, in the compound, tautomer, deuterated derivative, or pharmaceutically acceptable salt of the disclosure, wherein when Z is

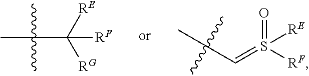

R^{E}, R^{F}, and R^{G} are each independently hydrogen, halogen, C₁-C₂ alkyl, —NR^{p}R^{q}, or —OR^{s}; wherein:
the C₁-C₆ alkyl of any one of R^{E}, R^{F}, and R^{G} is optionally substituted with 1 to 3 groups selected from cyano, —OH, and —OCH₃; wherein:
   R^{p} and R^{a}, for each occurrence, are each independently hydrogen or C₁-C₂ alkyl; and
   R^{s}, for each occurrence, is independently hydrogen or C₁-C₂ alkyl;
      and wherein all other variables not specifically defined herein are as defined in any one of preceding embodiments.

In some embodiments, in the compound, tautomer, deuterated derivative, or pharmaceutically acceptable salt of the disclosure, wherein:
when Z is

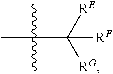

R^{E}, R^{F}, and R^{G} are each independently hydrogen, F, —CH₂CN, —OH, —OCH₃, —CH₃, —C₂H₅, or —CH₂OCH₃; and
when Z is

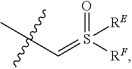

R^{E} and R^{F} are each independently —CH₃ or —NH₂; and wherein all other variables not specifically defined herein are as defined in any one of the preceding embodiments.

In some embodiments, the compound, tautomer, deuterated derivative, or pharmaceutically acceptable salt of the disclosure is represented by Formula (Va), Formula (Vb), or Formula (Vc):

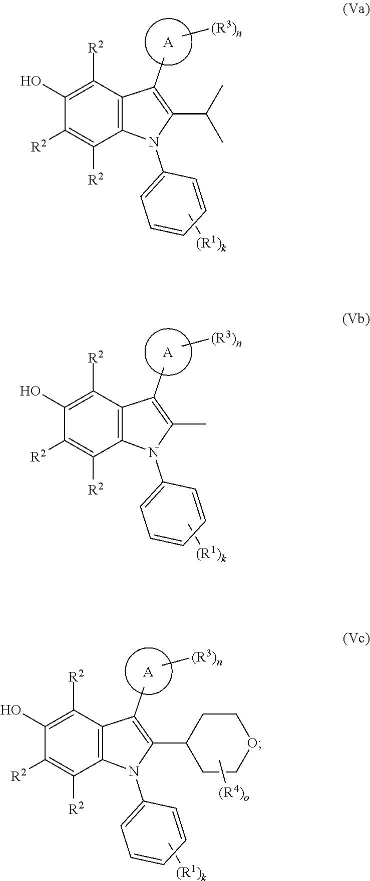

wherein all other variables are as defined for Formula (I) or in any one of the embodiments described above.

In some embodiments, the compound, tautomer, deuterated derivative, or pharmaceutically acceptable salt of the disclosure is represented by Formula (VIa), Formula (VIb), or Formula (VIc):

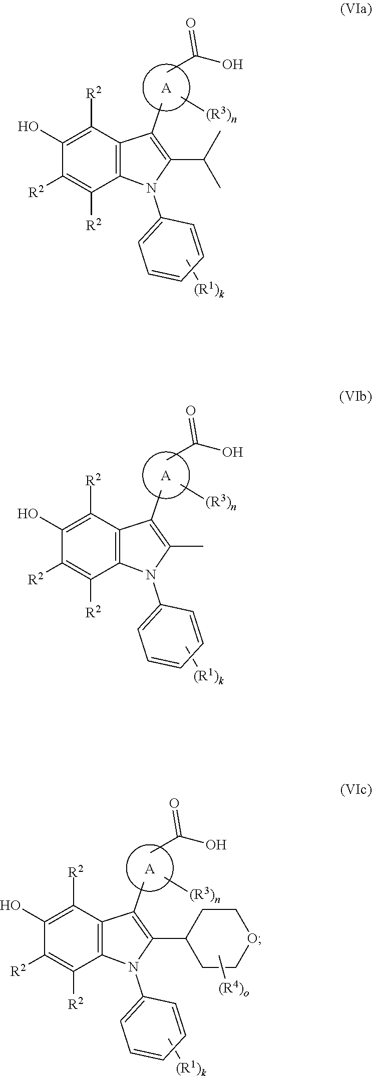

wherein n is an integer selected from 0, 1, and 2; and all other variables not specifically defined herein are as defined for Formula (I) or in any one of the embodiments described above.

In some embodiments, the compound, tautomer, deuterated derivative, or pharmaceutically acceptable salt of the disclosure is represented by Formula (VIIa), Formula (VIIb), Formula (VIIc), Formula (VIId), or Formula (VIIe):

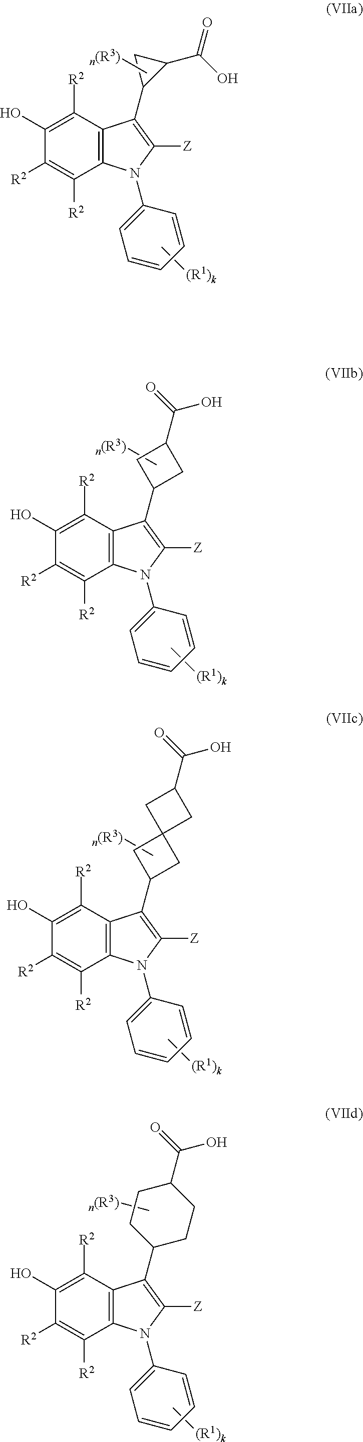

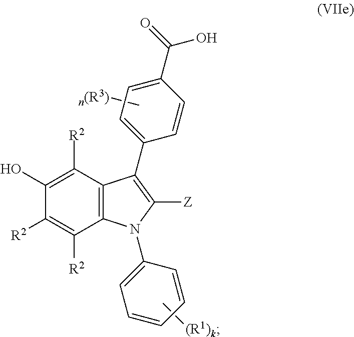

wherein n is an integer selected from 0, 1, and 2; and wherein all other variables not specifically defined herein are as defined for Formula (I) or any one of the embodiments described above.

In some embodiments of Formulae (I), (IIa)-(IIc), (III), (IV), (Va)-(Vc), (VIa)-(VIc), and (VIIa)-(VIIe), R², for each occurrence, is independently selected from hydrogen, halogen, cyano, C₁-C₄ alkyl, C₁-C₄ alkoxy, C₁-C₄ haloalkyl, —NR^{h}Rⁱ, and cyclopropyl; wherein R^{h} and Rⁱ, for each occurrence, is independently hydrogen or C₁-C₄ alkyl; and all other variables not specifically defined herein are as defined in any one of the preceding embodiments.

In some embodiments of Formulae (I), (IIa)-(IIc), (III), (IV), (Va)-(Vc), (VIa)-(VIc), and (VIIa)-(VIIe), R², for each occurrence, is independently selected from F, Cl, —CH₃, —NH₂, and cyclopropyl; and all other variables not specifically defined herein are as defined in any one of the preceding embodiments.

In some embodiments of Formulae (I), (IIa)-(IIc), (III), (IV), (Va)-(Vc), (VIa)-(VIc), and (VIIa)-(VIIe), R², for each occurrence, is independently selected from hydrogen, halogen, cyano, C₁-C₂ alkyl (optionally substituted with 1 to 3 groups selected from —CN, —OH, —OCH₃, and —NH₂), C₁-C₂ haloalkyl, and C₃-C₄ cycloalkyl; and all other variables not specifically defined herein are as defined in any one of the preceding embodiments.

In some embodiments, R³ for each occurrence in the compound, tautomer, deuterated derivative, or pharmaceutically acceptable salt of the disclosure, is independently selected from halogen, cyano, ═O, C₁-C₆ alkyl, C₁-C₆ alkoxy, C₁-C₆ haloalkyl, —C(═O)OR^{y}, —C(═O)NRs(═O)₂R^{y}, —S(═O)₂NR^{v}R^{w}, —S(═O)₂NR^{v}C(═O)R^{y}, —P(═O)R^{z}R^{z}, and 5 and 6-membered heteroaryl;
wherein the C₁-C₆ alkyl or the 5-membered heteroaryl of R³ is optionally substituted with 1 to 3 groups selected from cyano, —OR^{y}, and —NR^{v}R^{w}; wherein:
   R^{v} and R^{w}, for each occurrence, are each independently hydrogen or C₁-C₄ alkyl; and
   R^{y}, for each occurrence, is independently hydrogen or C₁-C₄ alkyl;
      and all other variables not specifically defined herein are as defined in any one of the preceding embodiments.

In some embodiments, R³ for each occurrence in the compound, tautomer, deuterated derivative, or pharmaceutically acceptable salt of the disclosure, is independently selected from halogen, cyano, ═O, C₁-C₄ alkyl, C₁-C₄ alkoxy, C₁-C₄ haloalkyl, —C(═O)OR^{y}, —C(═O)NRs(═O)₂R^{y}, —S(═O)₂NR^{v}R^{w}, —S(═O)₂NR^{v}C(═O)R^{y}, and 5-membered heteroaryl;
wherein the C₁-C₄ alkyl or the 5-membered heteroaryl of R³ is optionally substituted with 1 to 3 groups selected from cyano, —OR^{y}, and —NR^{v}R^{w}; wherein:
   R^{v} and R^{w}, for each occurrence, are each independently hydrogen or C₁-C₂ alkyl; and
   R^{y}, for each occurrence, is independently hydrogen or C₁-C₂ alkyl; and all other variables not specifically defined herein are as defined in any one of the preceding embodiments.

In some embodiments, R³ for each occurrence in the compound, tautomer, deuterated derivative, or pharmaceutically acceptable salt of the disclosure, is independently selected from halogen, cyano, ═O, C₁-C₂ alkyl, C₁-C₂ alkoxy, C₁-C₂ haloalkyl, —C(═O)OR^{y}, —C(═O)NRs(═O)₂R^{y}, —S(═O)₂NR^{v}R^{w}, —S(═O)₂NR^{v}C(═O)R^{y}, tetrazolyl, and oxadiazolyl;
wherein the C₁-C₂ alkyl of R³ is optionally substituted with 1 to 3 groups selected from cyano and —OH; wherein:
   R^{v} and R^{w}, for each occurrence, are each independently hydrogen or —CH₃; and
   R^{y}, for each occurrence, is independently hydrogen or —CH₃;
      and all other variables not specifically defined herein are as defined in any one of the preceding embodiments.

In some embodiments, R³ for each occurrence in the compound, tautomer, deuterated derivative, or pharmaceutically acceptable salt of the disclosure, is independently selected from F, cyano, ═O, —CH₃, —CH₂F, —CHF₂, —CF₃, —CH₂OH, —CH₂OCH₃, —OCH₃, —COOH, —CH₂COOH., —C(═O)NHS(═O)₂CH₃, —S(═O)₂NHCH₃, —S(═O)₂NHC(═O)CH₃, tetrazol-5-yl, 1,2,4-oxadiazol-5(4H)-onyl, and 1,3,4-oxadiazol-2(3H)-onyl; and all other variables not specifically defined herein are as defined in any one of the preceding embodiments.

In some embodiments, R⁴ for each occurrence in the compound, tautomer, deuterated derivative, or pharmaceutically acceptable salt of the disclosure, is independently selected from halogen, cyano, C₁-C₆ alkyl, C₁-C₆ alkoxy, C₁-C₆ haloalkyl, —C(═O)R^{y}, —C(═O)OR^{y}, —OR^{y}, and —S(═O)₂R^{y}; wherein:
wherein the C₁-C₆ alkyl of R⁴ is optionally substituted with 1 to 3 groups selected from cyano, —OR^{y}, —C(═O)OR^{y}, and —NR^{v}R^{w}; wherein:
   R^{v} and R^{w}, for each occurrence, are each independently hydrogen or C₁-C₆ alkyl; and
   R^{y}, for each occurrence, is independently hydrogen and C₁-C₄ alkyl; wherein:
      the C₁-C₄ alkyl of R^{y} is optionally substituted with 1 to 3 groups selected from halogen, cyano, —OH, —OCH₃, and —NH₂;
         and all other variables not specifically defined herein are as defined in any one of the preceding embodiments.

In some embodiments, R⁴, for each occurrence in the compound, tautomer, deuterated derivative, or pharmaceutically acceptable salt of the disclosure, is independently selected from halogen, cyano, C₁-C₆ alkyl, C₁-C₆ haloalkyl, —C(═O)R^{y}, —C(═O)OR^{y}, C(═O)NR^{v}R^{w}, —NR^{v}R^{w}, —OR^{y}, and —P(═O)R^{z}R^{z};
wherein R^{v} and R^{w}, for each occurrence, are each independently hydrogen or C₁-C₄ alkyl; and
   R^{y}, for each occurrence, is independently hydrogen or C₁-C₄ alkyl;
      and all other variables not specifically defined herein are as defined in any one of the preceding embodiments.

In some embodiments, R⁴ for each occurrence in the compound, tautomer, deuterated derivative, or pharmaceutically acceptable salt of the disclosure, is independently selected from halogen, cyano, C₁-C₄ alkyl, C₁-C₄ haloalkyl, —C(═O)R^{y}, —C(═O)OR^{y}, C(═O)NR^{v}R^{w}, —NR^{v}R^{W}, and —OR^{y}; wherein:
R^{v} and R^{w}, for each occurrence, are each independently hydrogen or C₁-C₂ alkyl; and
R^{y}, for each occurrence, is independently hydrogen or C₁-C₄ alkyl;
   and all other variables not specifically defined herein are as defined in any one of the preceding embodiments.

In some embodiments, R⁴ for each occurrence in the compound, tautomer, deuterated derivative, or pharmaceutically acceptable salt of the disclosure, is independently selected from halogen, cyano, C₁-C₂ alkyl, C₁-C₂ haloalkyl, —C(═O)OR^{y}, and —OR^{y}; wherein:
R^{y}, for each occurrence, is independently hydrogen or C₁-C₄ alkyl; and all other variables not specifically defined herein are as defined in any one of the preceding embodiments.

In some embodiments, R⁴ for each occurrence in the compound, tautomer, deuterated derivative, or pharmaceutically acceptable salt of the disclosure, is C(═O)OC(CH₃)₃; and all other variables not specifically defined herein are as defined in any one of the preceding embodiments.

In some embodiments, in the compound, tautomer, deuterated derivative, or pharmaceutically acceptable salt of the disclosure, m is 0; and all other variables not specifically defined herein are as defined in any one of the preceding embodiments.

In some embodiments, the compound of any one of Formulae (I), (IIa)-(IIc), (III), (IV), (Va)-(Vc), (VIa)-(VIc), and (VIIa)-(VIIe) is selected from Compounds 1-227 (Table I below) and tautomers of those compounds, deuterated derivatives of those tautomers and compounds, and pharmaceutically acceptable salt of any of the foregoing.

TABLE I
Compounds 1-227

| | |
|---|---|
| 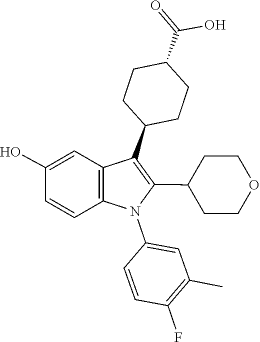 [SMILES: Cc1cc(-n2c(C3CCOCC3)c([C@H]3CC[C@H](C(=O)O)CC3)c3cc(O)ccc32)ccc1F] | 1 |
| 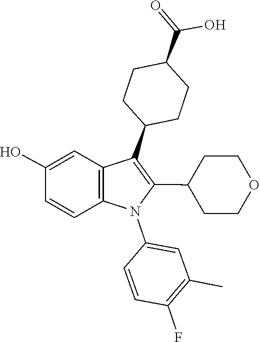 [SMILES: Cc1cc(-n2c(C3CCOCC3)c([C@H]3CC[C@@H](C(=O)O)CC3)c3cc(O)ccc32)ccc1F] | 2 |
| 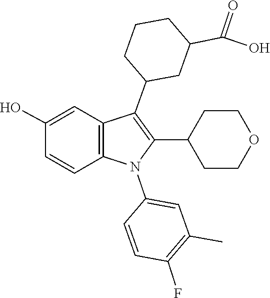 [SMILES: Cc1cc(-n2c(C3CCOCC3)c(C3CCCC(C(=O)O)C3)c3cc(O)ccc32)ccc1F] | 3 |
| 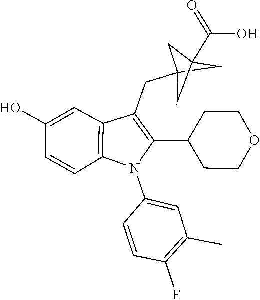 [SMILES: Cc1cc(-n2c(C3CCOCC3)c(CC34CC(C(=O)O)(C3)C4)c3cc(O)ccc32)ccc1F] | 4 |
| 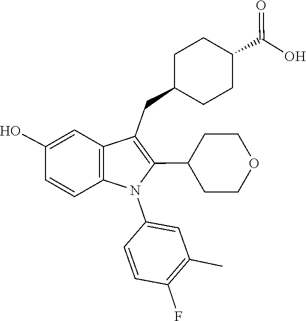 [SMILES: Cc1cc(-n2c(C3CCOCC3)c(C[C@H]3CC[C@H](C(=O)O)CC3)c3cc(O)ccc32)ccc1F] | 5 |
| 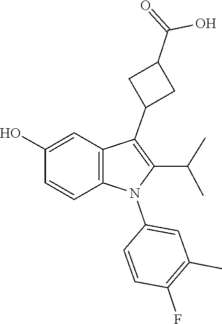 [SMILES: Cc1cc(-n2c(C(C)C)c(C3CC(C(=O)O)C3)c3cc(O)ccc32)ccc1F] | 6 |
| 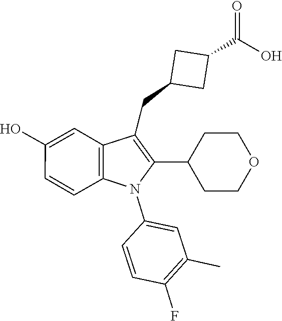 [SMILES: Cc1cc(-n2c(C3CCOCC3)c(C[C@H]3C[C@H](C(=O)O)C3)c3cc(O)ccc32)ccc1F] | 7 |
| 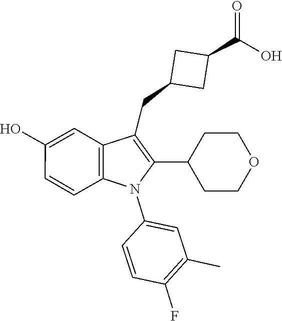 [SMILES: Cc1cc(-n2c(C3CCOCC3)c(C[C@H]3C[C@@H](C(=O)O)C3)c3cc(O)ccc32)ccc1F] | 8 |
| 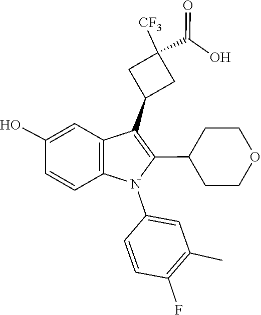 [SMILES: Cc1cc(-n2c(C3CCOCC3)c([C@H]3C[C@@](C(=O)O)(C(F)(F)F)C3)c3cc(O)ccc32)ccc1F] | 9 |
| 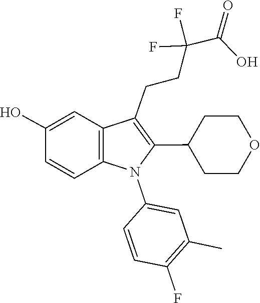 [SMILES: Cc1cc(-n2c(C3CCOCC3)c(CCC(F)(F)C(=O)O)c3cc(O)ccc32)ccc1F] | 10 |
| 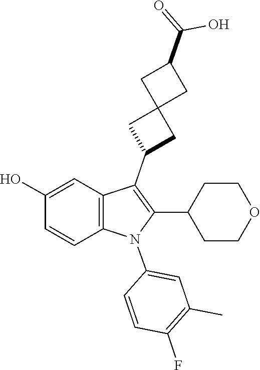 | 11 |
| 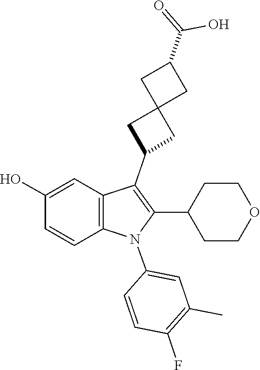 | 12 |
| 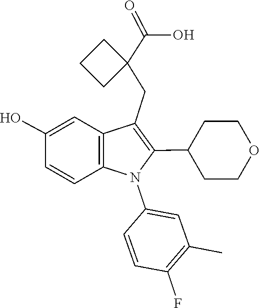 [SMILES: Cc1cc(-n2c(C3CCOCC3)c(CC3(C(=O)O)CCC3)c3cc(O)ccc32)ccc1F] | 13 |
| 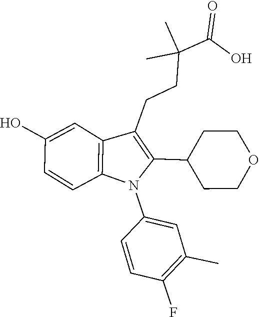 [SMILES: Cc1cc(-n2c(C3CCOCC3)c(CCC(C)(C)C(=O)O)c3cc(O)ccc32)ccc1F] | 14 |
| 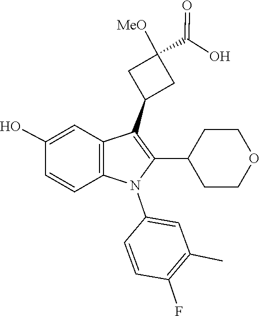 [SMILES: CO[C@]1(C(=O)O)C[C@H](c2c(C3CCOCC3)n(-c3ccc(F)c(C)c3)c3ccc(O)cc32)C1] | 15 |
| 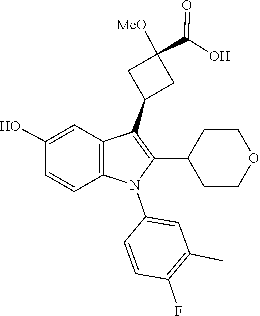 [SMILES: CO[C@]1(C(=O)O)C[C@@H](c2c(C3CCOCC3)n(-c3ccc(F)c(C)c3)c3ccc(O)cc32)C1] | 16 |
| 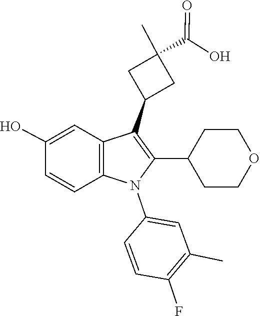 [SMILES: Cc1cc(-n2c(C3CCOCC3)c([C@H]3C[C@](C)(C(=O)O)C3)c3cc(O)ccc32)ccc1F] | 17 |
| 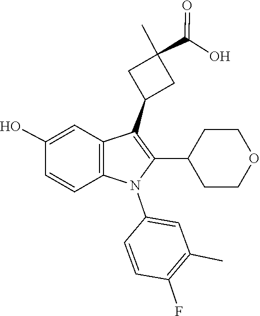 [SMILES: Cc1cc(-n2c(C3CCOCC3)c([C@H]3C[C@@](C)(C(=O)O)C3)c3cc(O)ccc32)ccc1F] | 18 |
| 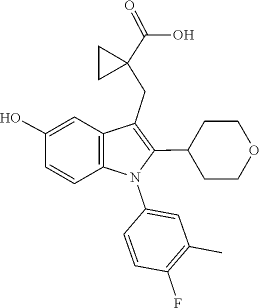 [SMILES: Cc1cc(-n2c(C3CCOCC3)c(CC3(C(=O)O)CC3)c3cc(O)ccc32)ccc1F] | 19 |
| 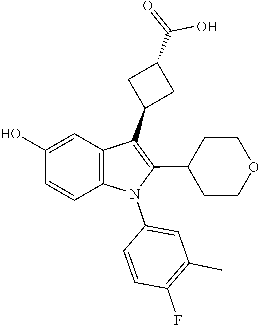 [SMILES: Cc1cc(-n2c(C3CCOCC3)c([C@H]3C[C@H](C(=O)O)C3)c3cc(O)ccc32)ccc1F] | 20 |
| 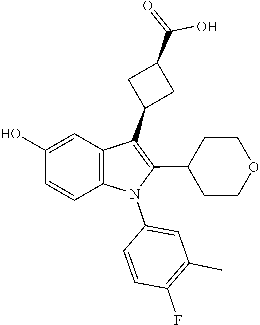 [SMILES: Cc1cc(-n2c(C3CCOCC3)c([C@H]3C[C@@H](C(=O)O)C3)c3cc(O)ccc32)ccc1F] | 21 |
| 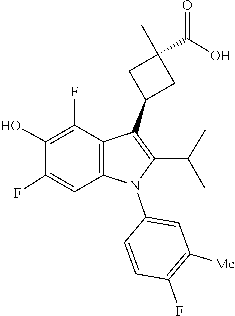 [SMILES: Cc1cc(-n2c(C(C)C)c([C@H]3C[C@](C)(C(=O)O)C3)c3c(F)c(O)c(F)cc32)ccc1F] | 22 |
| 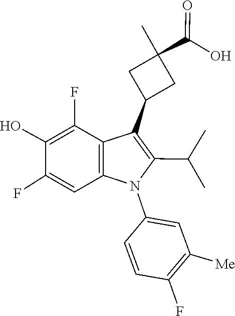 [SMILES: Cc1cc(-n2c(C(C)C)c([C@H]3C[C@@](C)(C(=O)O)C3)c3c(F)c(O)c(F)cc32)ccc1F] | 23 |
| 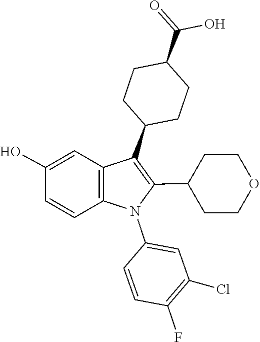 [SMILES: O=C(O)[C@H]1CC[C@@H](c2c(C3CCOCC3)n(-c3ccc(F)c(Cl)c3)c3ccc(O)cc32)CC1] | 24 |
| 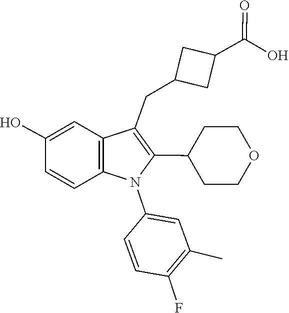 [SMILES: Cc1cc(-n2c(C3CCOCC3)c(CC3CC(C(=O)O)C3)c3cc(O)ccc32)ccc1F] | 25 |
| 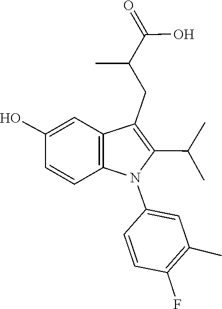 [SMILES: Cc1cc(-n2c(C(C)C)c(CC(C)C(=O)O)c3cc(O)ccc32)ccc1F] | 26 |
| 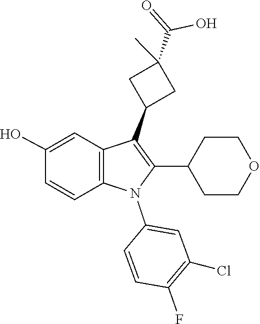 [SMILES: C[C@]1(C(=O)O)C[C@H](c2c(C3CCOCC3)n(-c3ccc(F)c(Cl)c3)c3ccc(O)cc32)C1] | 27 |
| 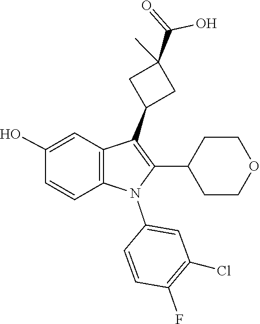 [SMILES: C[C@]1(C(=O)O)C[C@@H](c2c(C3CCOCC3)n(-c3ccc(F)c(Cl)c3)c3ccc(O)cc32)C1] | 28 |
| 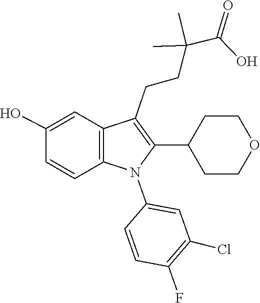 [SMILES: CC(C)(CCc1c(C2CCOCC2)n(-c2ccc(F)c(Cl)c2)c2ccc(O)cc12)C(=O)O] | 29 |
| 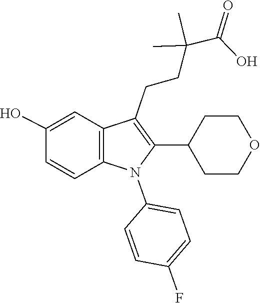 [SMILES: CC(C)(CCc1c(C2CCOCC2)n(-c2ccc(F)cc2)c2ccc(O)cc12)C(=O)O] | 30 |
| 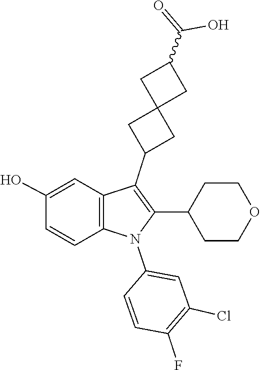 [SMILES: O=C(O)C1CC2(C1)CC(c1c(C3CCOCC3)n(-c3ccc(F)c(Cl)c3)c3ccc(O)cc13)C2] | 31 |
| 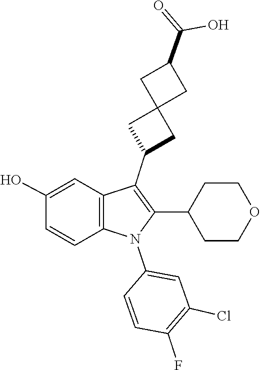 | 32 |
| 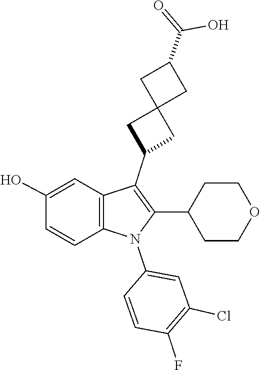 | 33 |
| 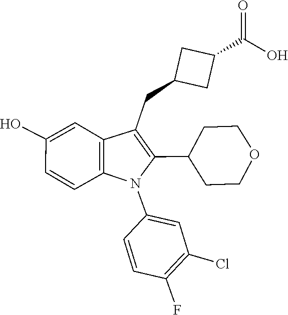 [SMILES: O=C(O)[C@H]1C[C@H](Cc2c(C3CCOCC3)n(-c3ccc(F)c(Cl)c3)c3ccc(O)cc23)C1] | 34 |
| 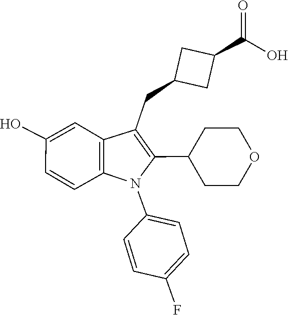 [SMILES: O=C(O)[C@H]1C[C@@H](Cc2c(C3CCOCC3)n(-c3ccc(F)cc3)c3ccc(O)cc23)C1] | 35 |
| 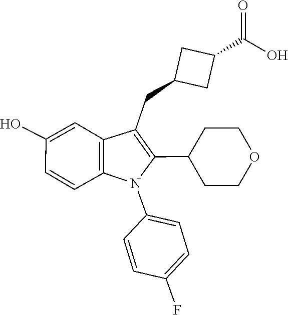 [SMILES: O=C(O)[C@H]1C[C@H](Cc2c(C3CCOCC3)n(-c3ccc(F)cc3)c3ccc(O)cc23)C1] | 36 |
| 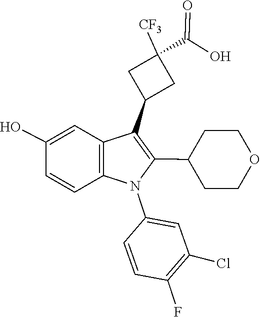 [SMILES: O=C(O)[C@]1(C(F)(F)F)C[C@@H](c2c(C3CCOCC3)n(-c3ccc(F)c(Cl)c3)c3ccc(O)cc32)C1] | 37 |
| 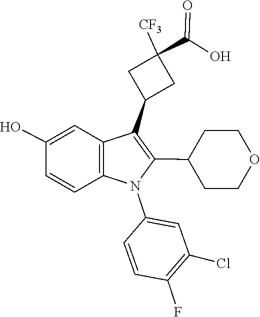 [SMILES: O=C(O)[C@]1(C(F)(F)F)C[C@H](c2c(C3CCOCC3)n(-c3ccc(F)c(Cl)c3)c3ccc(O)cc32)C1] | 38 |
| 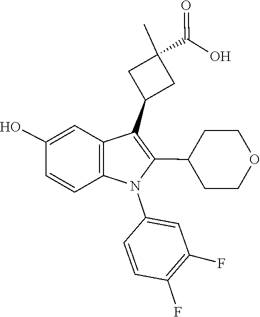 [SMILES: C[C@]1(C(=O)O)C[C@H](c2c(C3CCOCC3)n(-c3ccc(F)c(F)c3)c3ccc(O)cc32)C1] | 39 |
| 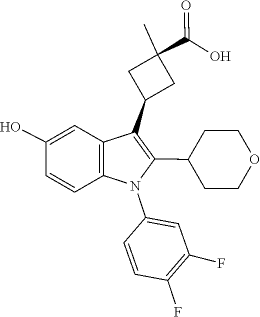 [SMILES: C[C@]1(C(=O)O)C[C@@H](c2c(C3CCOCC3)n(-c3ccc(F)c(F)c3)c3ccc(O)cc32)C1] | 40 |
| 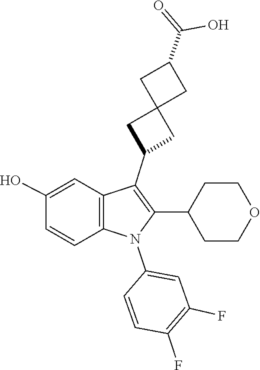 | 41 |
| 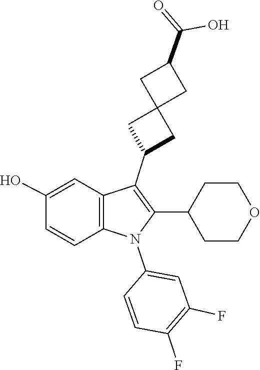 | 42 |
| 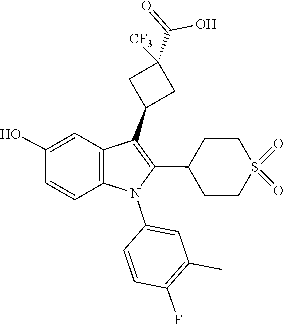 [SMILES: Cc1cc(-n2c(C3CCS(=O)(=O)CC3)c([C@H]3C[C@@](C(=O)O)(C(F)(F)F)C3)c3cc(O)ccc32)ccc1F] | 43 |
| 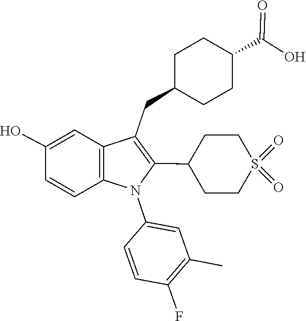 [SMILES: Cc1cc(-n2c(C3CCS(=O)(=O)CC3)c(C[C@H]3CC[C@H](C(=O)O)CC3)c3cc(O)ccc32)ccc1F] | 44 |
| 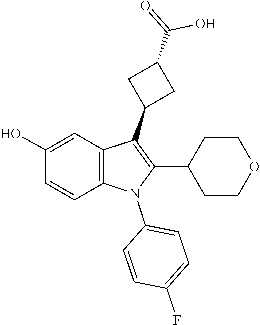 [SMILES: O=C(O)[C@H]1C[C@H](c2c(C3CCOCC3)n(-c3ccc(F)cc3)c3ccc(O)cc32)C1] | 45 |
| 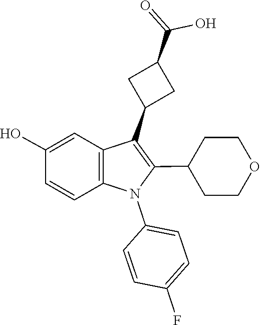 [SMILES: O=C(O)[C@H]1C[C@@H](c2c(C3CCOCC3)n(-c3ccc(F)cc3)c3ccc(O)cc32)C1] | 46 |
| 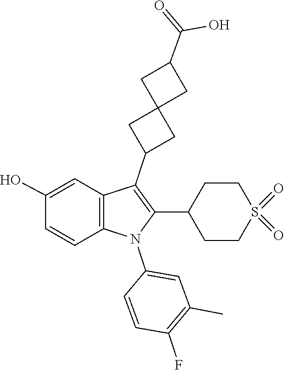 [SMILES: Cc1cc(-n2c(C3CCS(=O)(=O)CC3)c(C3CC4(CC(C(=O)O)C4)C3)c3cc(O)ccc32)ccc1F] | 47 |
| 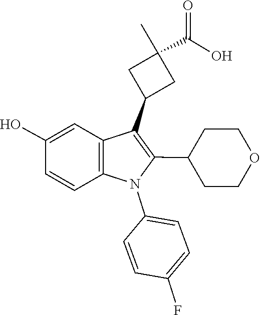 [SMILES: C[C@]1(C(=O)O)C[C@H](c2c(C3CCOCC3)n(-c3ccc(F)cc3)c3ccc(O)cc32)C1] | 48 |
| 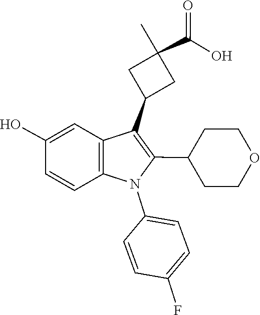 [SMILES: C[C@]1(C(=O)O)C[C@@H](c2c(C3CCOCC3)n(-c3ccc(F)cc3)c3ccc(O)cc32)C1] | 49 |
| 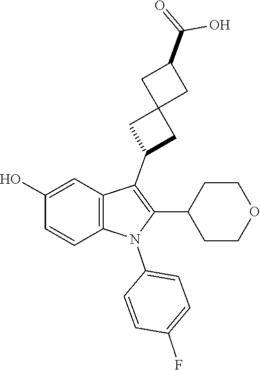 | 50 |
| 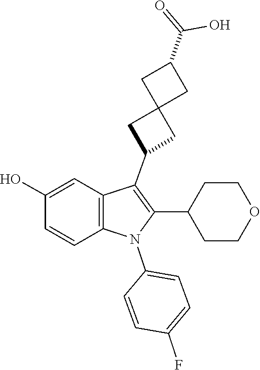 | 51 |
| 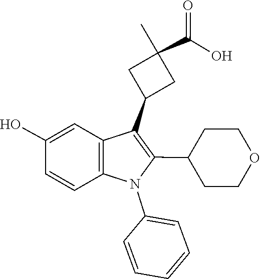 [SMILES: C[C@]1(C(=O)O)C[C@@H](c2c(C3CCOCC3)n(-c3ccccc3)c3ccc(O)cc32)C1] | 52 |
| 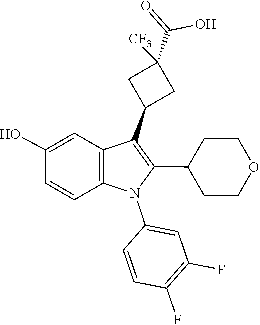 [SMILES: O=C(O)[C@]1(C(F)(F)F)C[C@@H](c2c(C3CCOCC3)n(-c3ccc(F)c(F)c3)c3ccc(O)cc32)C1] | 53 |
| 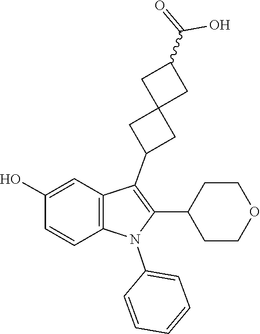 [SMILES: O=C(O)C1CC2(C1)CC(c1c(C3CCOCC3)n(-c3ccccc3)c3ccc(O)cc13)C2] | 54 |
| 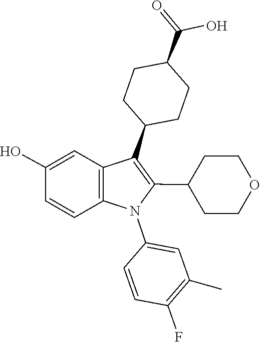 [SMILES: Cc1cc(-n2c(C3CCOCC3)c([C@H]3CC[C@@H](C(=O)O)CC3)c3cc(O)ccc32)ccc1F] | 55 |
| 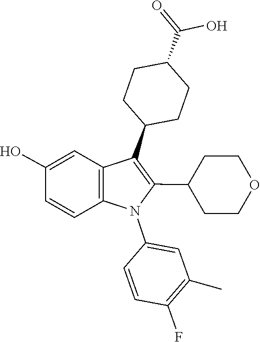 [SMILES: Cc1cc(-n2c(C3CCOCC3)c([C@H]3CC[C@H](C(=O)O)CC3)c3cc(O)ccc32)ccc1F] | 56 |
| 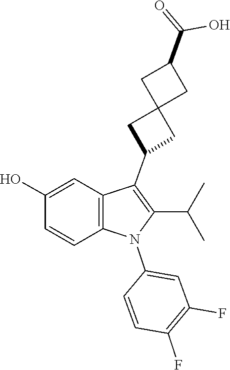 | 57 |
| 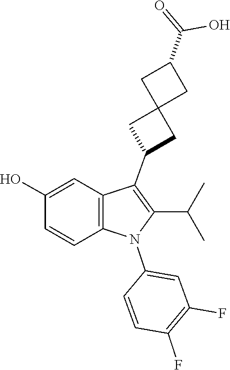 | 58 |
| 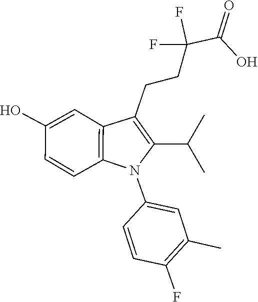 [SMILES: Cc1cc(-n2c(C(C)C)c(CCC(F)(F)C(=O)O)c3cc(O)ccc32)ccc1F] | 59 |
| 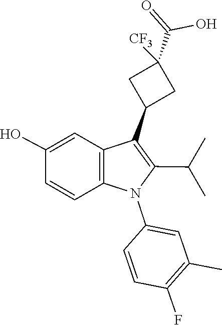 [SMILES: Cc1cc(-n2c(C(C)C)c([C@H]3C[C@@](C(=O)O)(C(F)(F)F)C3)c3cc(O)ccc32)ccc1F] | 60 |
| 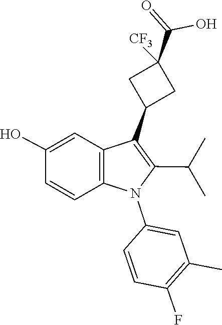 [SMILES: Cc1cc(-n2c(C(C)C)c([C@H]3C[C@](C(=O)O)(C(F)(F)F)C3)c3cc(O)ccc32)ccc1F] | 61 |
| 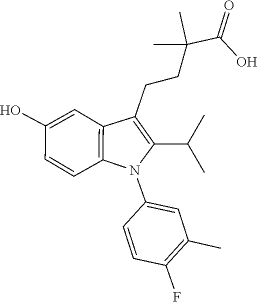 [SMILES: Cc1cc(-n2c(C(C)C)c(CCC(C)(C)C(=O)O)c3cc(O)ccc32)ccc1F] | 62 |
| 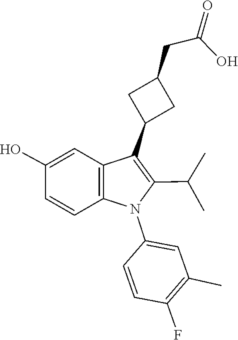 [SMILES: Cc1cc(-n2c(C(C)C)c([C@H]3C[C@@H](CC(=O)O)C3)c3cc(O)ccc32)ccc1F] | 63 |
| 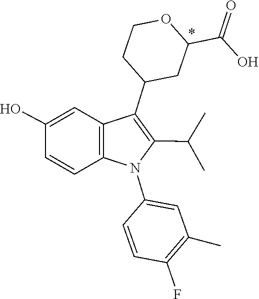 [SMILES: Cc1cc(-n2c(C(C)C)c(C3CCOC(C(=O)O)C3)c3cc(O)ccc32)ccc1F] | 64 |
| 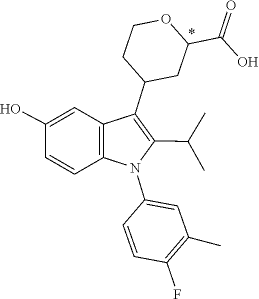 [SMILES: Cc1cc(-n2c(C(C)C)c(C3CCOC(C(=O)O)C3)c3cc(O)ccc32)ccc1F] | 65 |
| 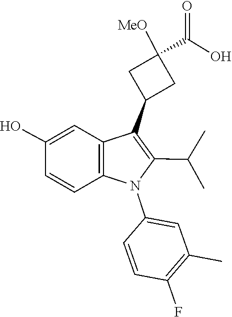 [SMILES: CO[C@]1(C(=O)O)C[C@H](c2c(C(C)C)n(-c3ccc(F)c(C)c3)c3ccc(O)cc32)C1] | 66 |
| 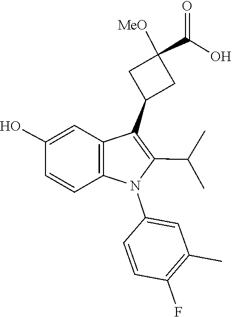 [SMILES: CO[C@]1(C(=O)O)C[C@@H](c2c(C(C)C)n(-c3ccc(F)c(C)c3)c3ccc(O)cc32)C1] | 67 |
| 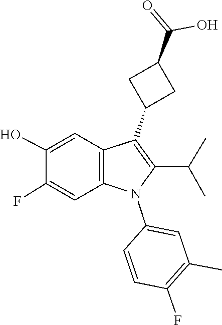 [SMILES: Cc1cc(-n2c(C(C)C)c([C@H]3C[C@H](C(=O)O)C3)c3cc(O)c(F)cc32)ccc1F] | 68 |
| 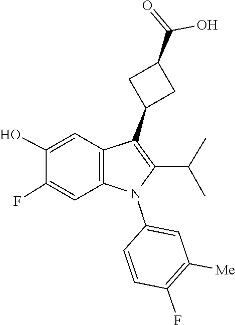 [SMILES: Cc1cc(-n2c(C(C)C)c([C@H]3C[C@@H](C(=O)O)C3)c3cc(O)c(F)cc32)ccc1F] | 69 |
| 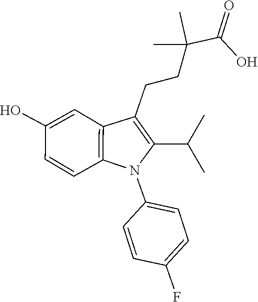 [SMILES: CC(C)c1c(CCC(C)(C)C(=O)O)c2cc(O)ccc2n1-c1ccc(F)cc1] | 70 |
| 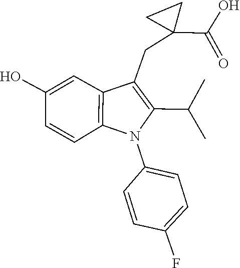 [SMILES: CC(C)c1c(CC2(C(=O)O)CC2)c2cc(O)ccc2n1-c1ccc(F)cc1] | 71 |
| 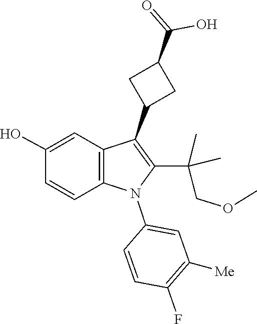 | 72 |
| 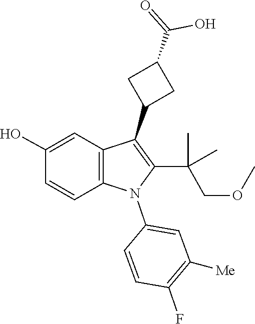 | 73 |
| 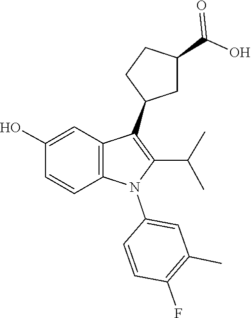 [SMILES: Cc1cc(-n2c(C(C)C)c([C@H]3CC[C@@H](C(=O)O)C3)c3cc(O)ccc32)ccc1F] | 74 |
| 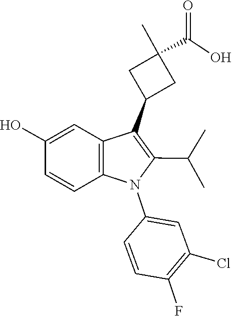 [SMILES: CC(C)c1c([C@H]2C[C@](C)(C(=O)O)C2)c2cc(O)ccc2n1-c1ccc(F)c(Cl)c1] | 75 |
| 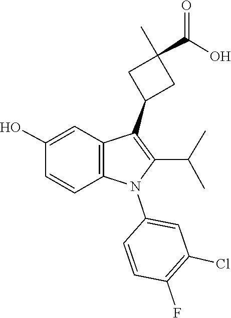 [SMILES: CC(C)c1c([C@H]2C[C@@](C)(C(=O)O)C2)c2cc(O)ccc2n1-c1ccc(F)c(Cl)c1] | 76 |
| 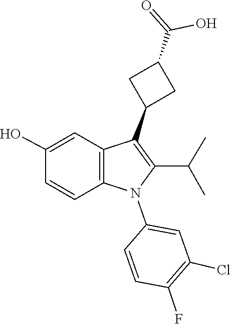 [SMILES: CC(C)c1c([C@H]2C[C@H](C(=O)O)C2)c2cc(O)ccc2n1-c1ccc(F)c(Cl)c1] | 77 |
| 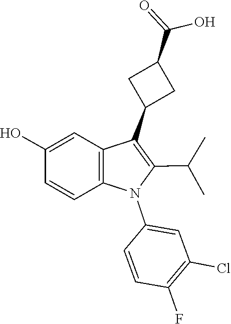 [SMILES: CC(C)c1c([C@H]2C[C@@H](C(=O)O)C2)c2cc(O)ccc2n1-c1ccc(F)c(Cl)c1] | 78 |
| 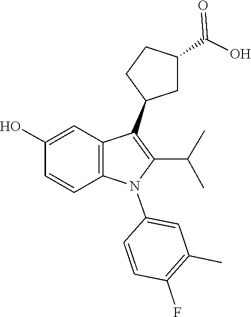 [SMILES: Cc1cc(-n2c(C(C)C)c([C@H]3CC[C@H](C(=O)O)C3)c3cc(O)ccc32)ccc1F] | 79 |
| 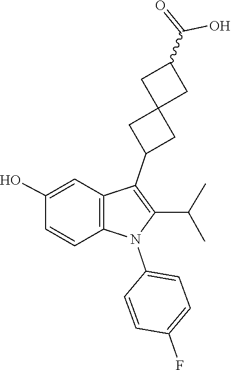 [SMILES: CC(C)c1c(C2CC3(CC(C(=O)O)C3)C2)c2cc(O)ccc2n1-c1ccc(F)cc1] | 80 |
| 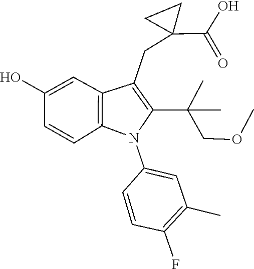 [SMILES: COCC(C)(C)c1c(CC2(C(=O)O)CC2)c2cc(O)ccc2n1-c1ccc(F)c(C)c1] | 81 |
| 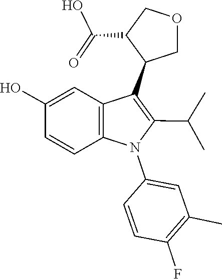 | 82 |
| 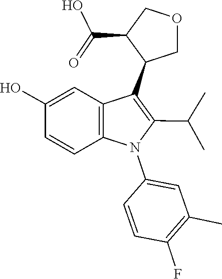 [SMILES: Cc1cc(-n2c(C(C)C)c([C@@H]3COC[C@@H]3C(=O)O)c3cc(O)ccc32)ccc1F] | 83 |
| 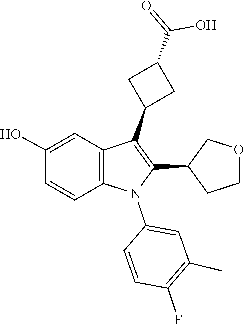 [SMILES: Cc1cc(-n2c([C@@H]3CCOC3)c([C@H]3C[C@H](C(=O)O)C3)c3cc(O)ccc32)ccc1F] | 84 |
| 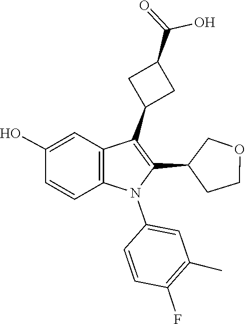 [SMILES: Cc1cc(-n2c([C@@H]3CCOC3)c([C@H]3C[C@@H](C(=O)O)C3)c3cc(O)ccc32)ccc1F] | 85 |
| 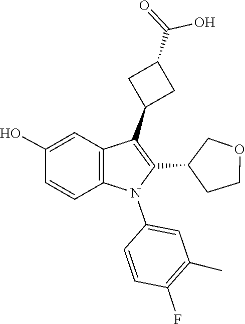 [SMILES: Cc1cc(-n2c([C@H]3CCOC3)c([C@H]3C[C@H](C(=O)O)C3)c3cc(O)ccc32)ccc1F] | 86 |
| 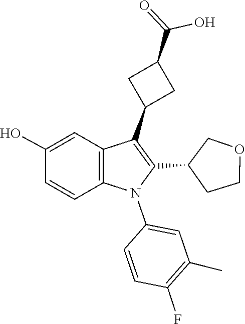 [SMILES: Cc1cc(-n2c([C@H]3CCOC3)c([C@H]3C[C@@H](C(=O)O)C3)c3cc(O)ccc32)ccc1F] | 87 |
| 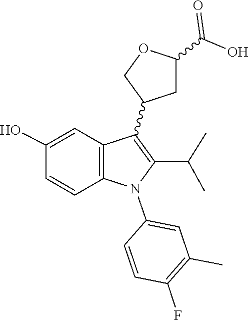 [SMILES: Cc1cc(-n2c(C(C)C)c(C3COC(C(=O)O)C3)c3cc(O)ccc32)ccc1F] | 88 |
| 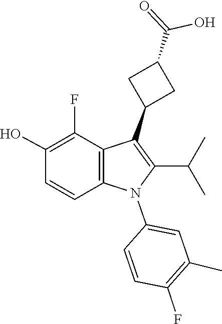 [SMILES: Cc1cc(-n2c(C(C)C)c([C@H]3C[C@H](C(=O)O)C3)c3c(F)c(O)ccc32)ccc1F] | 89 |
| 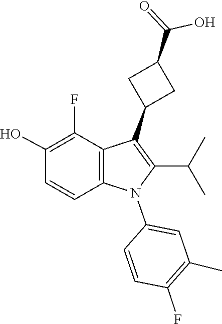 [SMILES: Cc1cc(-n2c(C(C)C)c([C@H]3C[C@@H](C(=O)O)C3)c3c(F)c(O)ccc32)ccc1F] | 90 |
| 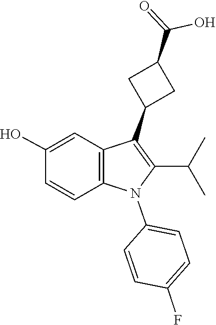 [SMILES: CC(C)c1c([C@H]2C[C@@H](C(=O)O)C2)c2cc(O)ccc2n1-c1ccc(F)cc1] | 91 |
| 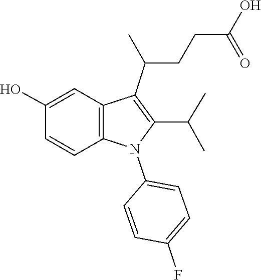 [SMILES: CC(C)c1c(C(C)CCC(=O)O)c2cc(O)ccc2n1-c1ccc(F)cc1] | 92 |
| 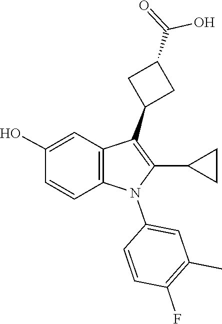 [SMILES: Cc1cc(-n2c(C3CC3)c([C@H]3C[C@H](C(=O)O)C3)c3cc(O)ccc32)ccc1F] | 93 |
| 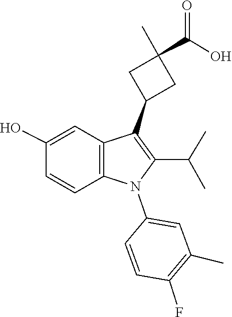 [SMILES: Cc1cc(-n2c(C(C)C)c([C@H]3C[C@@](C)(C(=O)O)C3)c3cc(O)ccc32)ccc1F] | 94 |
| 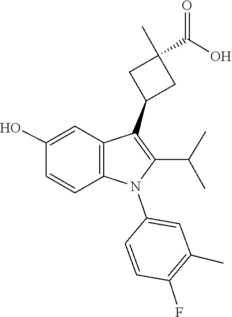 [SMILES: Cc1cc(-n2c(C(C)C)c([C@H]3C[C@](C)(C(=O)O)C3)c3cc(O)ccc32)ccc1F] | 95 |
| 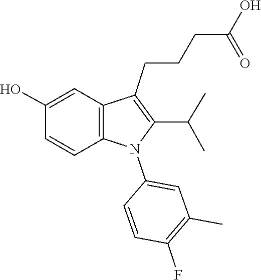 [SMILES: Cc1cc(-n2c(C(C)C)c(CCCC(=O)O)c3cc(O)ccc32)ccc1F] | 96 |
| 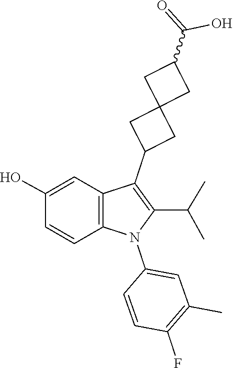 [SMILES: Cc1cc(-n2c(C(C)C)c(C3CC4(CC(C(=O)O)C4)C3)c3cc(O)ccc32)ccc1F] | 97 |
| 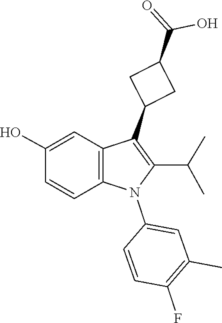 [SMILES: Cc1cc(-n2c(C(C)C)c([C@H]3C[C@@H](C(=O)O)C3)c3cc(O)ccc32)ccc1F] | 98 |
| 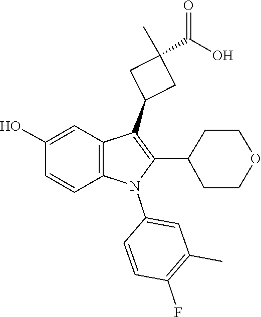 [SMILES: Cc1cc(-n2c(C3CCOCC3)c([C@H]3C[C@](C)(C(=O)O)C3)c3cc(O)ccc32)ccc1F] | 99 |
| 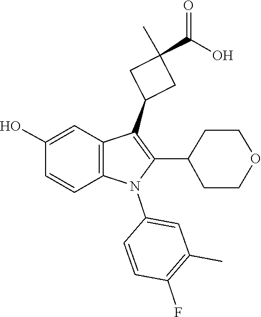 [SMILES: Cc1cc(-n2c(C3CCOCC3)c([C@H]3C[C@@](C)(C(=O)O)C3)c3cc(O)ccc32)ccc1F] | 100 |
| 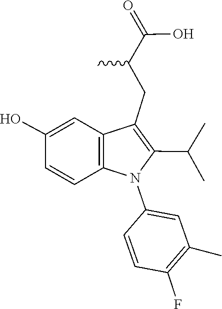 [SMILES: Cc1cc(-n2c(C(C)C)c(CC(C)C(=O)O)c3cc(O)ccc32)ccc1F] | 101 |
| 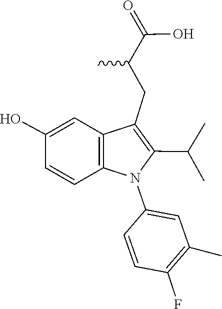 [SMILES: Cc1cc(-n2c(C(C)C)c(CC(C)C(=O)O)c3cc(O)ccc32)ccc1F] | 102 |
| 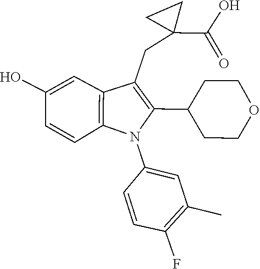 [SMILES: Cc1cc(-n2c(C3CCOCC3)c(CC3(C(=O)O)CC3)c3cc(O)ccc32)ccc1F] | 103 |
| 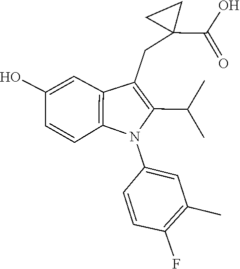 [SMILES: Cc1cc(-n2c(C(C)C)c(CC3(C(=O)O)CC3)c3cc(O)ccc32)ccc1F] | 104 |
| 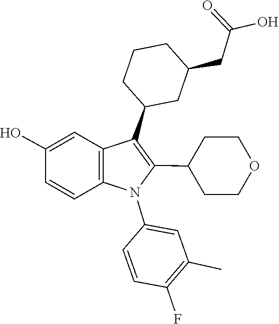 [SMILES: Cc1cc(-n2c(C3CCOCC3)c([C@H]3CCC[C@@H](CC(=O)O)C3)c3cc(O)ccc32)ccc1F] | 105 |
| 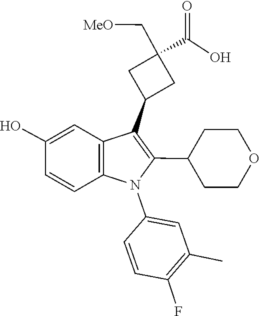 [SMILES: COC[C@]1(C(=O)O)C[C@H](c2c(C3CCOCC3)n(-c3ccc(F)c(C)c3)c3ccc(O)cc32)C1] | 106 |
| 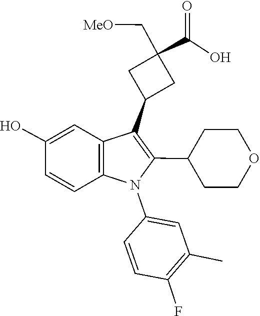 [SMILES: COC[C@]1(C(=O)O)C[C@@H](c2c(C3CCOCC3)n(-c3ccc(F)c(C)c3)c3ccc(O)cc32)C1] | 107 |
| 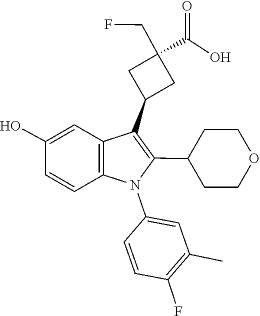 [SMILES: Cc1cc(-n2c(C3CCOCC3)c([C@H]3C[C@](CF)(C(=O)O)C3)c3cc(O)ccc32)ccc1F] | 108 |
| 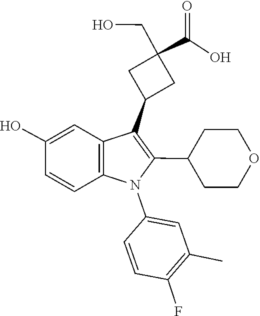 [SMILES: Cc1cc(-n2c(C3CCOCC3)c([C@H]3C[C@@](CO)(C(=O)O)C3)c3cc(O)ccc32)ccc1F] | 109 |
| 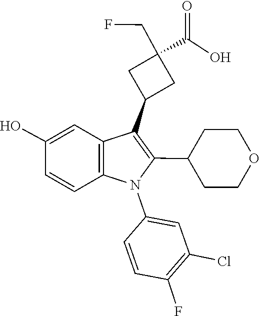 [SMILES: O=C(O)[C@]1(CF)C[C@@H](c2c(C3CCOCC3)n(-c3ccc(F)c(Cl)c3)c3ccc(O)cc32)C1] | 110 |
| 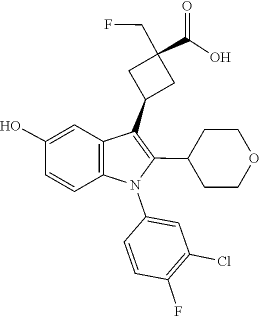 [SMILES: O=C(O)[C@]1(CF)C[C@H](c2c(C3CCOCC3)n(-c3ccc(F)c(Cl)c3)c3ccc(O)cc32)C1] | 111 |
| 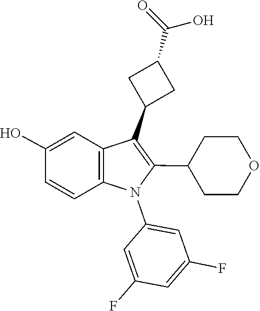 [SMILES: O=C(O)[C@H]1C[C@H](c2c(C3CCOCC3)n(-c3cc(F)cc(F)c3)c3ccc(O)cc32)C1] | 112 |
| 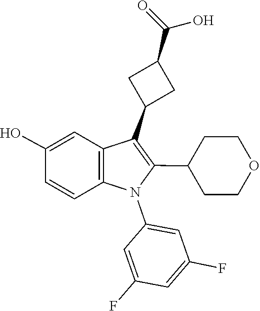 [SMILES: O=C(O)[C@H]1C[C@@H](c2c(C3CCOCC3)n(-c3cc(F)cc(F)c3)c3ccc(O)cc32)C1] | 113 |
| 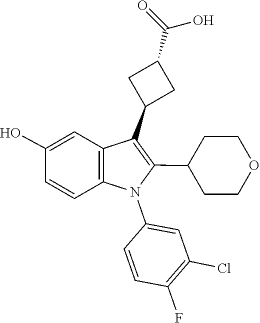 [SMILES: O=C(O)[C@H]1C[C@H](c2c(C3CCOCC3)n(-c3ccc(F)c(Cl)c3)c3ccc(O)cc32)C1] | 114 |
| 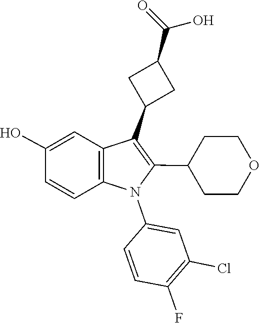 [SMILES: O=C(O)[C@H]1C[C@@H](c2c(C3CCOCC3)n(-c3ccc(F)c(Cl)c3)c3ccc(O)cc32)C1] | 115 |
| 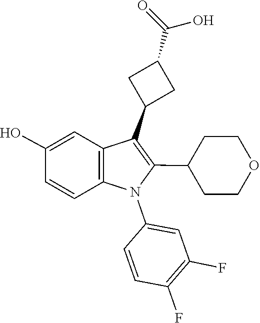 [SMILES: O=C(O)[C@H]1C[C@H](c2c(C3CCOCC3)n(-c3ccc(F)c(F)c3)c3ccc(O)cc32)C1] | 116 |
| 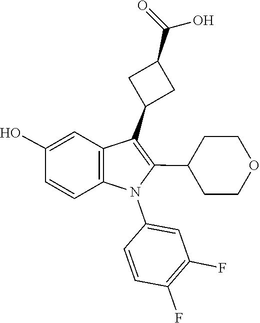 [SMILES: O=C(O)[C@H]1C[C@@H](c2c(C3CCOCC3)n(-c3ccc(F)c(F)c3)c3ccc(O)cc32)C1] | 117 |
| 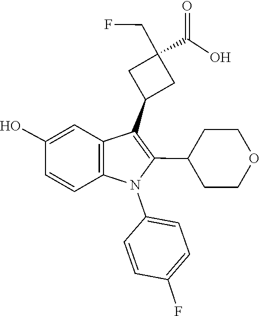 [SMILES: O=C(O)[C@]1(CF)C[C@@H](c2c(C3CCOCC3)n(-c3ccc(F)cc3)c3ccc(O)cc32)C1] | 118 |
| 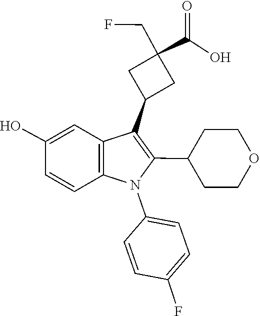 [SMILES: O=C(O)[C@]1(CF)C[C@H](c2c(C3CCOCC3)n(-c3ccc(F)cc3)c3ccc(O)cc32)C1] | 119 |
| 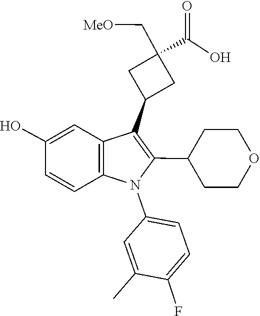 [SMILES: COC[C@]1(C(=O)O)C[C@H](c2c(C3CCOCC3)n(-c3ccc(F)c(C)c3)c3ccc(O)cc32)C1] | 120 |
| 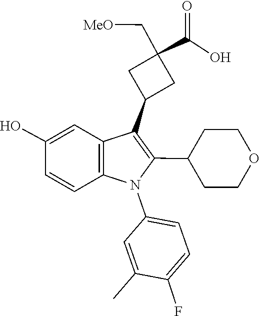 [SMILES: COC[C@]1(C(=O)O)C[C@@H](c2c(C3CCOCC3)n(-c3ccc(F)c(C)c3)c3ccc(O)cc32)C1] | 121 |
| 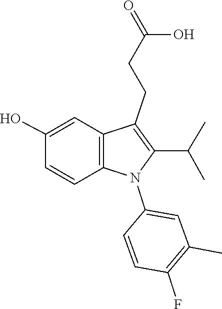 [SMILES: Cc1cc(-n2c(C(C)C)c(CCC(=O)O)c3cc(O)ccc32)ccc1F] | 122 |
| 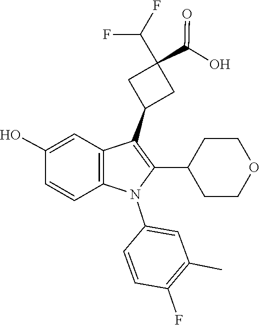 [SMILES: Cc1cc(-n2c(C3CCOCC3)c([C@H]3C[C@](C(=O)O)(C(F)F)C3)c3cc(O)ccc32)ccc1F] | 123 |
| 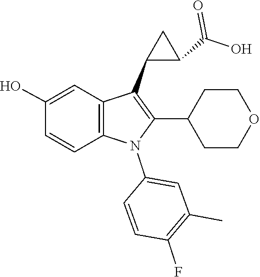 [SMILES: Cc1cc(-n2c(C3CCOCC3)c([C@H]3C[C@@H]3C(=O)O)c3cc(O)ccc32)ccc1F] | 124 |
| 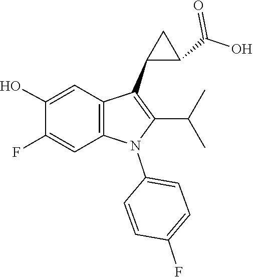 [SMILES: CC(C)c1c([C@H]2C[C@@H]2C(=O)O)c2cc(O)c(F)cc2n1-c1ccc(F)cc1] | 125 |
| 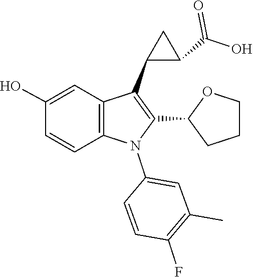 [SMILES: Cc1cc(-n2c([C@H]3CCCO3)c([C@H]3C[C@@H]3C(=O)O)c3cc(O)ccc32)ccc1F] | 126 |
| 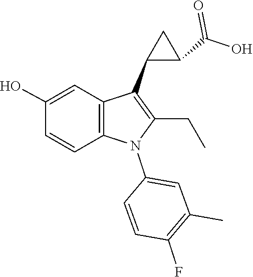 [SMILES: CCc1c([C@H]2C[C@@H]2C(=O)O)c2cc(O)ccc2n1-c1ccc(F)c(C)c1] | 127 |
| 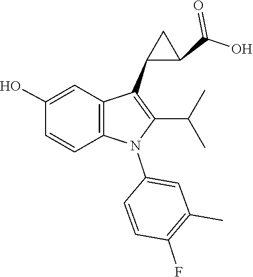 [SMILES: Cc1cc(-n2c(C(C)C)c([C@H]3C[C@H]3C(=O)O)c3cc(O)ccc32)ccc1F] | 128 |
| 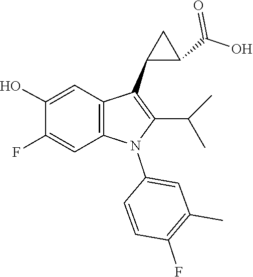 [SMILES: Cc1cc(-n2c(C(C)C)c([C@H]3C[C@@H]3C(=O)O)c3cc(O)c(F)cc32)ccc1F] | 129 |
| 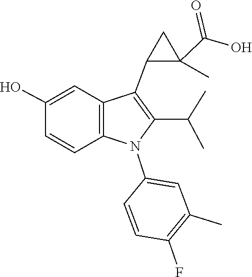 [SMILES: Cc1cc(-n2c(C(C)C)c(C3CC3(C)C(=O)O)c3cc(O)ccc32)ccc1F] | 130 |
| 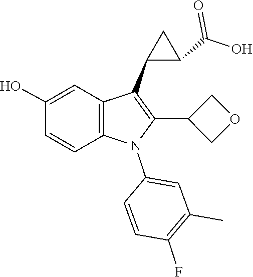 [SMILES: Cc1cc(-n2c(C3COC3)c([C@H]3C[C@@H]3C(=O)O)c3cc(O)ccc32)ccc1F] | 131 |
| 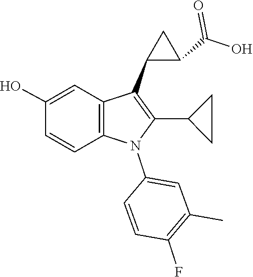 [SMILES: Cc1cc(-n2c(C3CC3)c([C@H]3C[C@@H]3C(=O)O)c3cc(O)ccc32)ccc1F] | 132 |
| 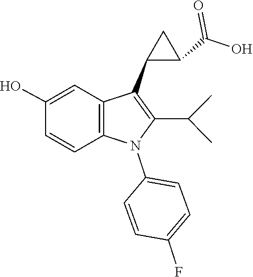 [SMILES: CC(C)c1c([C@H]2C[C@@H]2C(=O)O)c2cc(O)ccc2n1-c1ccc(F)cc1] | 133 |
| 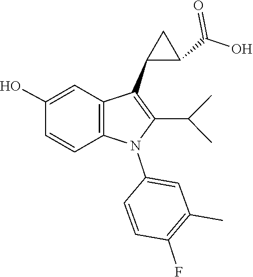 [SMILES: Cc1cc(-n2c(C(C)C)c([C@H]3C[C@@H]3C(=O)O)c3cc(O)ccc32)ccc1F] | 134 |
| 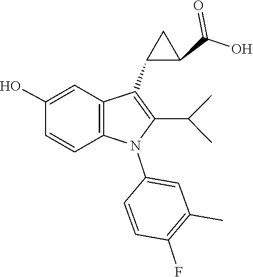 [SMILES: Cc1cc(-n2c(C(C)C)c([C@@H]3C[C@H]3C(=O)O)c3cc(O)ccc32)ccc1F] | 135 |
| 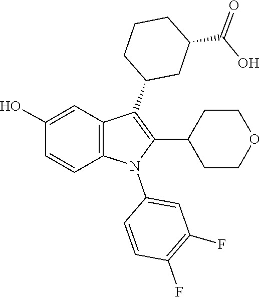 [SMILES: O=C(O)[C@H]1CCC[C@@H](c2c(C3CCOCC3)n(-c3ccc(F)c(F)c3)c3ccc(O)cc23)C1] | 136 |
| 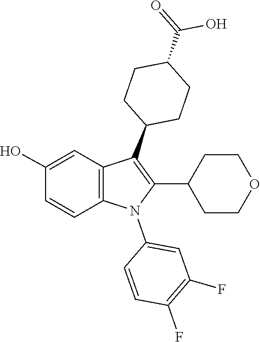 [SMILES: O=C(O)[C@H]1CC[C@H](c2c(C3CCOCC3)n(-c3ccc(F)c(F)c3)c3ccc(O)cc32)CC1] | 137 |
| 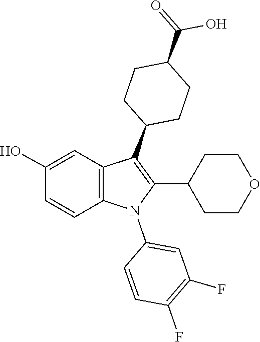 [SMILES: O=C(O)[C@H]1CC[C@@H](c2c(C3CCOCC3)n(-c3ccc(F)c(F)c3)c3ccc(O)cc32)CC1] | 138 |
| 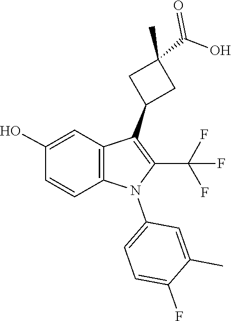 [SMILES: Cc1cc(-n2c(C(F)(F)F)c([C@H]3C[C@](C)(C(=O)O)C3)c3cc(O)ccc32)ccc1F] | 139 |
| 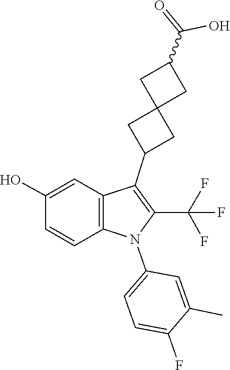 [SMILES: Cc1cc(-n2c(C(F)(F)F)c(C3CC4(CC(C(=O)O)C4)C3)c3cc(O)ccc32)ccc1F] | 140 |
| 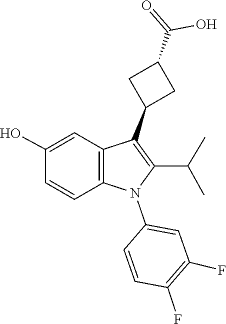 [SMILES: CC(C)c1c([C@H]2C[C@H](C(=O)O)C2)c2cc(O)ccc2n1-c1ccc(F)c(F)c1] | 141 |
| 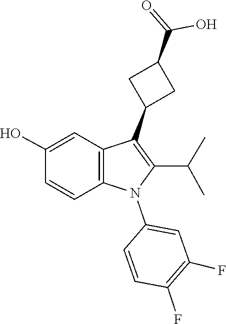 [SMILES: CC(C)c1c([C@H]2C[C@@H](C(=O)O)C2)c2cc(O)ccc2n1-c1ccc(F)c(F)c1] | 142 |
| 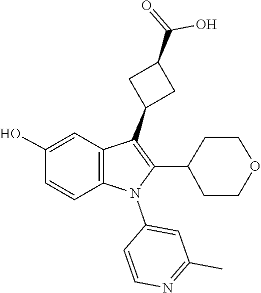 [SMILES: Cc1cc(-n2c(C3CCOCC3)c([C@H]3C[C@@H](C(=O)O)C3)c3cc(O)ccc32)ccn1] | 143 |
| 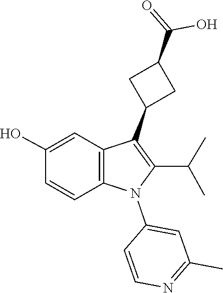 [SMILES: Cc1cc(-n2c(C(C)C)c([C@H]3C[C@@H](C(=O)O)C3)c3cc(O)ccc32)ccn1] | 144 |
| 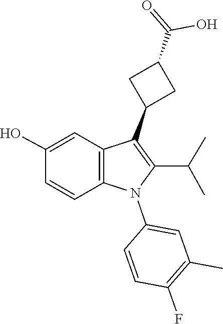 [SMILES: Cc1cc(-n2c(C(C)C)c([C@H]3C[C@H](C(=O)O)C3)c3cc(O)ccc32)ccc1F] | 145 |
| 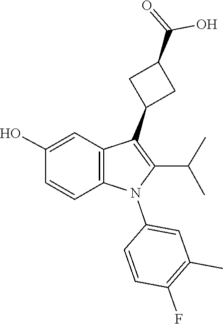 [SMILES: Cc1cc(-n2c(C(C)C)c([C@H]3C[C@@H](C(=O)O)C3)c3cc(O)ccc32)ccc1F] | 146 |
| 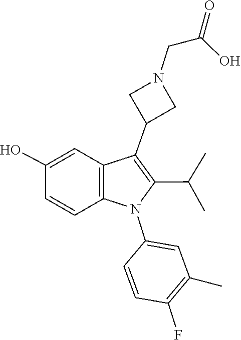 [SMILES: Cc1cc(-n2c(C(C)C)c(C3CN(CC(=O)O)C3)c3cc(O)ccc32)ccc1F] | 147 |
| 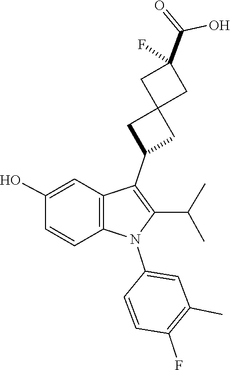 | 148 |
| 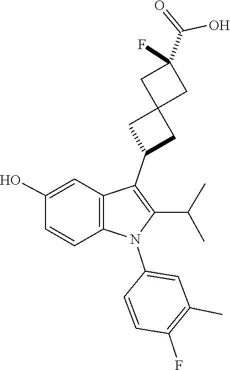 | 149 |
| 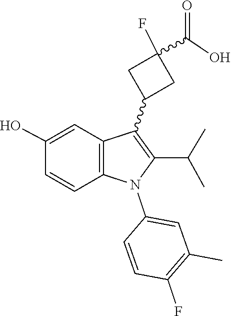 [SMILES: Cc1cc(-n2c(C(C)C)c(C3CC(F)(C(=O)O)C3)c3cc(O)ccc32)ccc1F] | 150 |
| 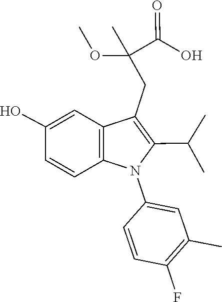 [SMILES: COC(C)(Cc1c(C(C)C)n(-c2ccc(F)c(C)c2)c2ccc(O)cc12)C(=O)O] | 151 |
| 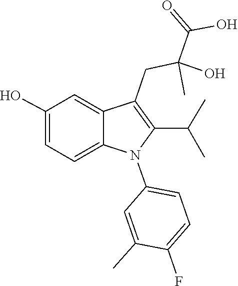 [SMILES: Cc1cc(-n2c(C(C)C)c(CC(C)(O)C(=O)O)c3cc(O)ccc32)ccc1F] | 152 |
| 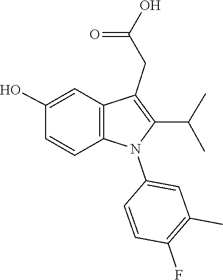 [SMILES: Cc1cc(-n2c(C(C)C)c(CC(=O)O)c3cc(O)ccc32)ccc1F] | 153 |
| 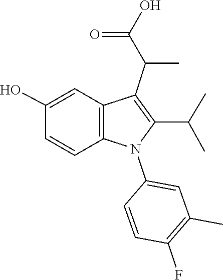 [SMILES: Cc1cc(-n2c(C(C)C)c(C(C)C(=O)O)c3cc(O)ccc32)ccc1F] | 154 |
| 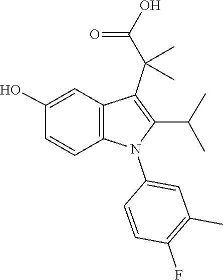 [SMILES: Cc1cc(-n2c(C(C)C)c(C(C)(C)C(=O)O)c3cc(O)ccc32)ccc1F] | 155 |
| 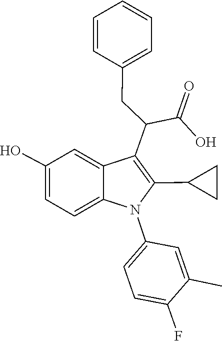 [SMILES: Cc1cc(-n2c(C3CC3)c(C(Cc3ccccc3)C(=O)O)c3cc(O)ccc32)ccc1F] | 156 |
| 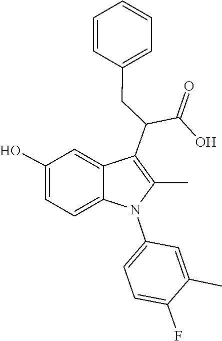 [SMILES: Cc1cc(-n2c(C)c(C(Cc3ccccc3)C(=O)O)c3cc(O)ccc32)ccc1F] | 157 |
| 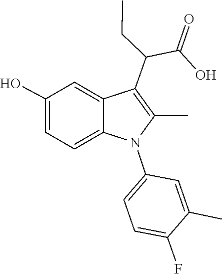 [SMILES: CCC(C(=O)O)c1c(C)n(-c2ccc(F)c(C)c2)c2ccc(O)cc12] | 158 |
| 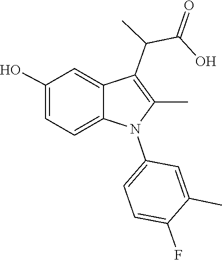 [SMILES: Cc1cc(-n2c(C)c(C(C)C(=O)O)c3cc(O)ccc32)ccc1F] | 159 |
| 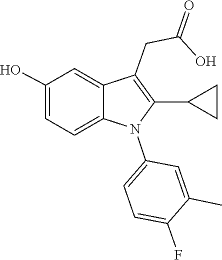 [SMILES: Cc1cc(-n2c(C3CC3)c(CC(=O)O)c3cc(O)ccc32)ccc1F] | 160 |
| 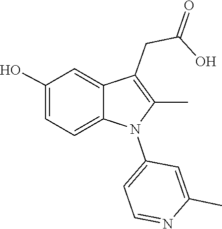 [SMILES: Cc1cc(-n2c(C)c(CC(=O)O)c3cc(O)ccc32)ccn1] | 161 |
| 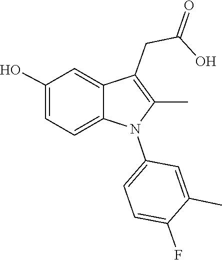 [SMILES: Cc1cc(-n2c(C)c(CC(=O)O)c3cc(O)ccc32)ccc1F] | 162 |
| 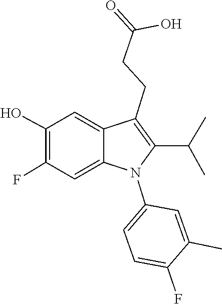 [SMILES: Cc1cc(-n2c(C(C)C)c(CCC(=O)O)c3cc(O)c(F)cc32)ccc1F] | 163 |
| 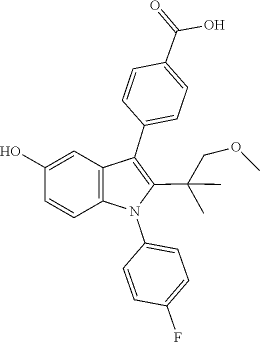 [SMILES: COCC(C)(C)c1c(-c2ccc(C(=O)O)cc2)c2cc(O)ccc2n1-c1ccc(F)cc1] | 164 |
| 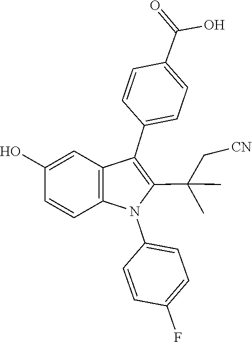 [SMILES: CC(C)(CC#N)c1c(-c2ccc(C(=O)O)cc2)c2cc(O)ccc2n1-c1ccc(F)cc1] | 165 |
| 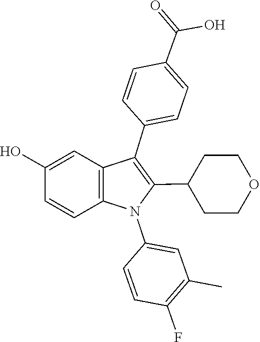 [SMILES: Cc1cc(-n2c(C3CCOCC3)c(-c3ccc(C(=O)O)cc3)c3cc(O)ccc32)ccc1F] | 166 |
| 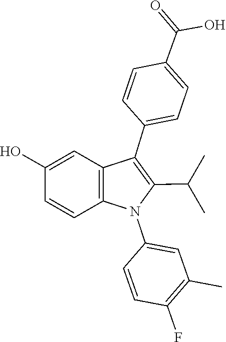 [SMILES: Cc1cc(-n2c(C(C)C)c(-c3ccc(C(=O)O)cc3)c3cc(O)ccc32)ccc1F] | 167 |
| 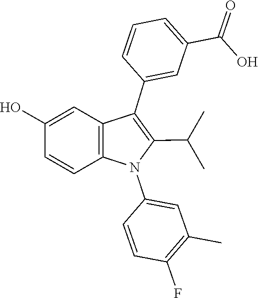 [SMILES: Cc1cc(-n2c(C(C)C)c(-c3cccc(C(=O)O)c3)c3cc(O)ccc32)ccc1F] | 168 |
| 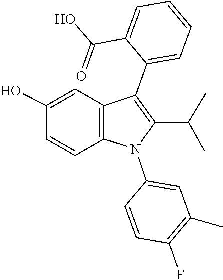 [SMILES: Cc1cc(-n2c(C(C)C)c(-c3ccccc3C(=O)O)c3cc(O)ccc32)ccc1F] | 169 |
| 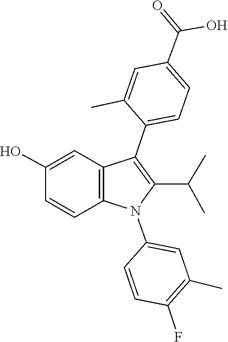 [SMILES: Cc1cc(-n2c(C(C)C)c(-c3ccc(C(=O)O)cc3C)c3cc(O)ccc32)ccc1F] | 170 |
| 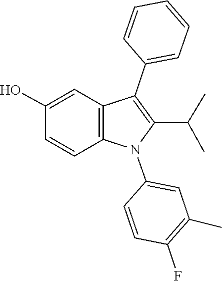 [SMILES: Cc1cc(-n2c(C(C)C)c(-c3ccccc3)c3cc(O)ccc32)ccc1F] | 171 |
| 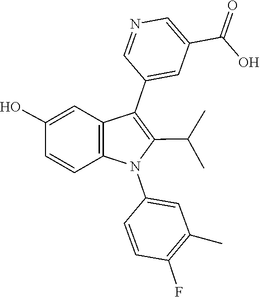 [SMILES: Cc1cc(-n2c(C(C)C)c(-c3cncc(C(=O)O)c3)c3cc(O)ccc32)ccc1F] | 172 |
| 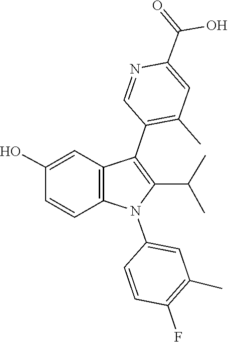 [SMILES: Cc1cc(-n2c(C(C)C)c(-c3cnc(C(=O)O)cc3C)c3cc(O)ccc32)ccc1F] | 173 |
| 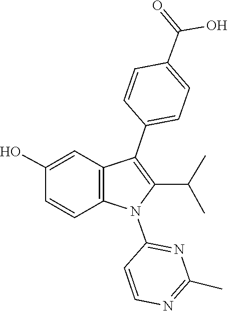 [SMILES: Cc1nccc(-n2c(C(C)C)c(-c3ccc(C(=O)O)cc3)c3cc(O)ccc32)n1] | 174 |
| 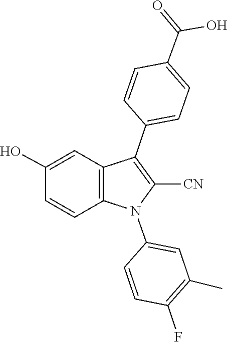 [SMILES: Cc1cc(-n2c(C#N)c(-c3ccc(C(=O)O)cc3)c3cc(O)ccc32)ccc1F] | 175 |
| 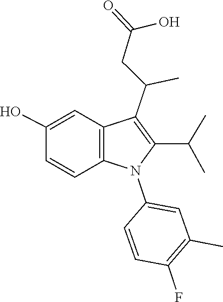 [SMILES: Cc1cc(-n2c(C(C)C)c(C(C)CC(=O)O)c3cc(O)ccc32)ccc1F] | 176 |
| 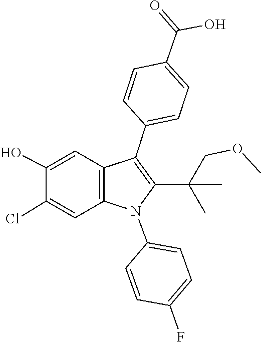 [SMILES: COCC(C)(C)c1c(-c2ccc(C(=O)O)cc2)c2cc(O)c(Cl)cc2n1-c1ccc(F)cc1] | 177 |
| 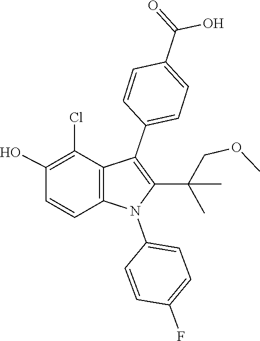 [SMILES: COCC(C)(C)c1c(-c2ccc(C(=O)O)cc2)c2c(Cl)c(O)ccc2n1-c1ccc(F)cc1] | 178 |
| 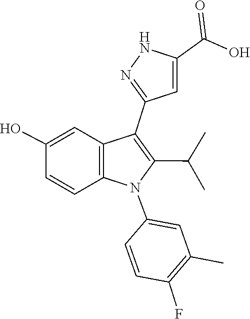 [SMILES: Cc1cc(-n2c(C(C)C)c(-c3cc(C(=O)O)[nH]n3)c3cc(O)ccc32)ccc1F] | 179 |
| 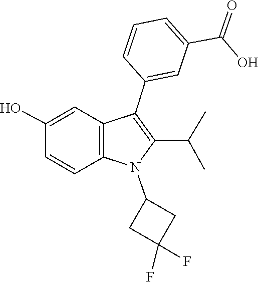 [SMILES: CC(C)c1c(-c2cccc(C(=O)O)c2)c2cc(O)ccc2n1C1CC(F)(F)C1] | 180 |
| 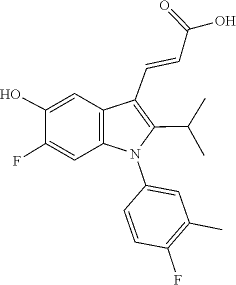 [SMILES: Cc1cc(-n2c(C(C)C)c(/C=C/C(=O)O)c3cc(O)c(F)cc32)ccc1F] | 181 |
| 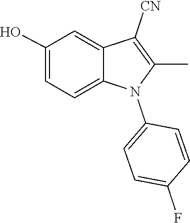 [SMILES: Cc1c(C#N)c2cc(O)ccc2n1-c1ccc(F)cc1] | 182 |
| 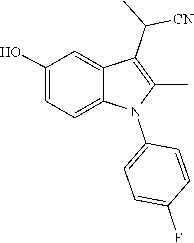 [SMILES: Cc1c(C(C)C#N)c2cc(O)ccc2n1-c1ccc(F)cc1] | 183 |
| 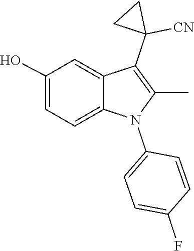 [SMILES: Cc1c(C2(C#N)CC2)c2cc(O)ccc2n1-c1ccc(F)cc1] | 184 |
| 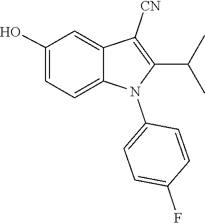 [SMILES: CC(C)c1c(C#N)c2cc(O)ccc2n1-c1ccc(F)cc1] | 185 |
| 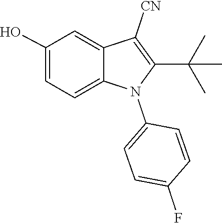 [SMILES: CC(C)(C)c1c(C#N)c2cc(O)ccc2n1-c1ccc(F)cc1] | 186 |
| 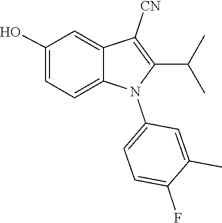 [SMILES: Cc1cc(-n2c(C(C)C)c(C#N)c3cc(O)ccc32)ccc1F] | 187 |
| 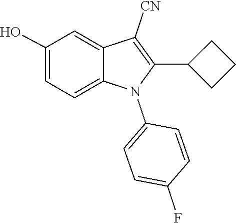 [SMILES: N#Cc1c(C2CCC2)n(-c2ccc(F)cc2)c2ccc(O)cc12] | 188 |
| 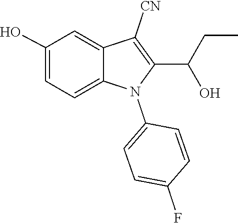 [SMILES: CCC(O)c1c(C#N)c2cc(O)ccc2n1-c1ccc(F)cc1] | 189 |
| 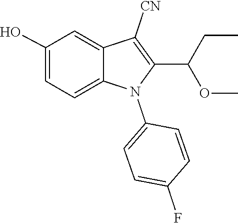 [SMILES: CCC(OC)c1c(C#N)c2cc(O)ccc2n1-c1ccc(F)cc1] | 190 |
| 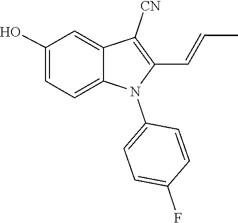 [SMILES: C/C=C/c1c(C#N)c2cc(O)ccc2n1-c1ccc(F)cc1] | 191 |
| 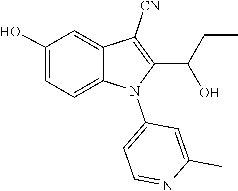 [SMILES: CCC(O)c1c(C#N)c2cc(O)ccc2n1-c1ccnc(C)c1] | 192 |
| 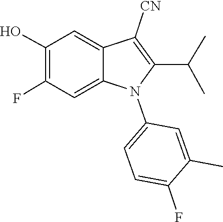 [SMILES: Cc1cc(-n2c(C(C)C)c(C#N)c3cc(O)c(F)cc32)ccc1F] | 193 |
| 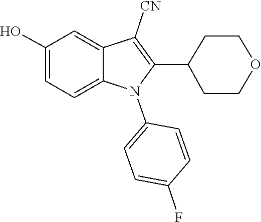 [SMILES: N#Cc1c(C2CCOCC2)n(-c2ccc(F)cc2)c2ccc(O)cc12] | 194 |
| 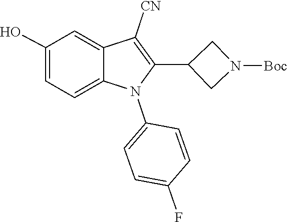 [SMILES: CC(C)(C)OC(=O)N1CC(c2c(C#N)c3cc(O)ccc3n2-c2ccc(F)cc2)C1] | 195 |
| 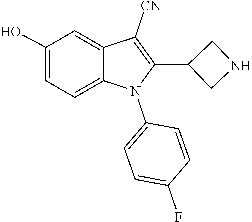 [SMILES: N#Cc1c(C2CNC2)n(-c2ccc(F)cc2)c2ccc(O)cc12] | 196 |
| 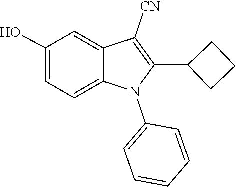 [SMILES: N#Cc1c(C2CCC2)n(-c2ccccc2)c2ccc(O)cc12] | 197 |
| 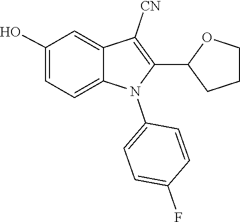 [SMILES: N#Cc1c(C2CCCO2)n(-c2ccc(F)cc2)c2ccc(O)cc12] | 198 |
| 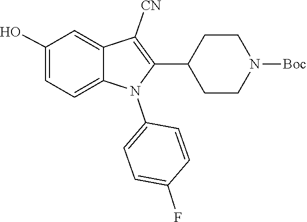 [SMILES: CC(C)(C)OC(=O)N1CCC(c2c(C#N)c3cc(O)ccc3n2-c2ccc(F)cc2)CC1] | 199 |
| 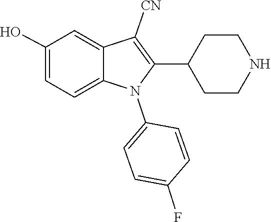 [SMILES: N#Cc1c(C2CCNCC2)n(-c2ccc(F)cc2)c2ccc(O)cc12] | 200 |
| 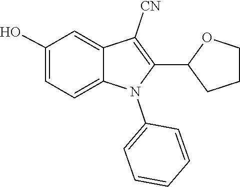 [SMILES: N#Cc1c(C2CCCO2)n(-c2ccccc2)c2ccc(O)cc12] | 201 |
| 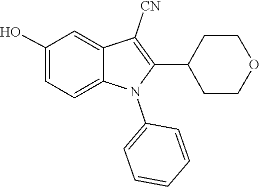 [SMILES: N#Cc1c(C2CCOCC2)n(-c2ccccc2)c2ccc(O)cc12] | 202 |
| 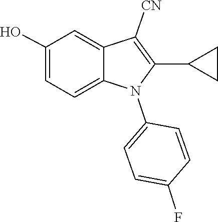 [SMILES: N#Cc1c(C2CC2)n(-c2ccc(F)cc2)c2ccc(O)cc12] | 203 |
| 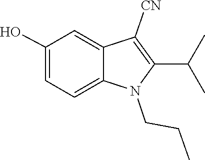 [SMILES: CCCn1c(C(C)C)c(C#N)c2cc(O)ccc21] | 204 |
| 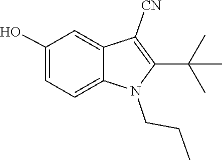 [SMILES: CCCn1c(C(C)(C)C)c(C#N)c2cc(O)ccc21] | 205 |
| 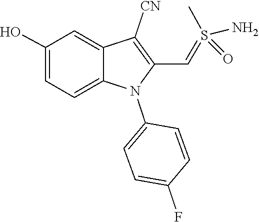 [SMILES: CS(N)(=O)=Cc1c(C#N)c2cc(O)ccc2n1-c1ccc(F)cc1] | 206 |
| 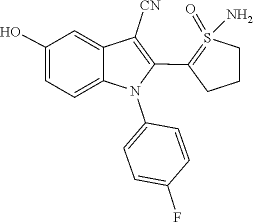 [SMILES: N#Cc1c(C2=S(N)(=O)CCC2)n(-c2ccc(F)cc2)c2ccc(O)cc12] | 207 |
| 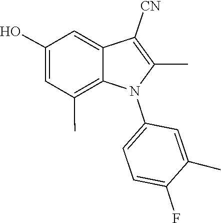 [SMILES: Cc1cc(-n2c(C)c(C#N)c3cc(O)cc(C)c32)ccc1F] | 208 |
| 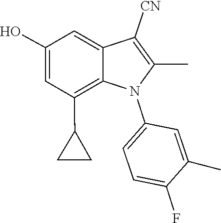 [SMILES: Cc1cc(-n2c(C)c(C#N)c3cc(O)cc(C4CC4)c32)ccc1F] | 209 |
| 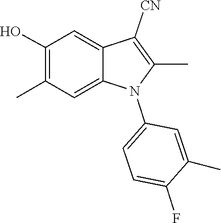 [SMILES: Cc1cc2c(cc1O)c(C#N)c(C)n2-c1ccc(F)c(C)c1] | 210 |
| 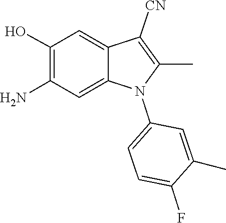 [SMILES: Cc1cc(-n2c(C)c(C#N)c3cc(O)c(N)cc32)ccc1F] | 211 |
| 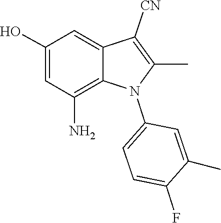 [SMILES: Cc1cc(-n2c(C)c(C#N)c3cc(O)cc(N)c32)ccc1F] | 212 |
| 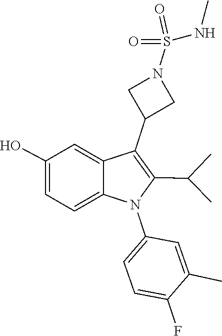 [SMILES: CNS(=O)(=O)N1CC(c2c(C(C)C)n(-c3ccc(F)c(C)c3)c3ccc(O)cc23)C1] | 213 |
| 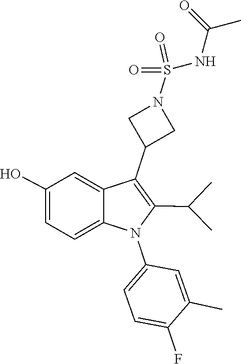 [SMILES: CC(=O)NS(=O)(=O)N1CC(c2c(C(C)C)n(-c3ccc(F)c(C)c3)c3ccc(O)cc23)C1] | 214 |
| 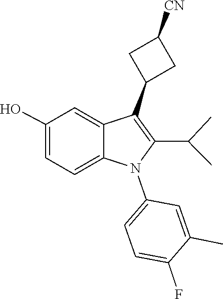 [SMILES: Cc1cc(-n2c(C(C)C)c([C@H]3C[C@@H](C#N)C3)c3cc(O)ccc32)ccc1F] | 215 |
| 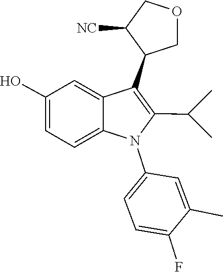 [SMILES: Cc1cc(-n2c(C(C)C)c([C@@H]3COC[C@@H]3C#N)c3cc(O)ccc32)ccc1F] | 216 |
| 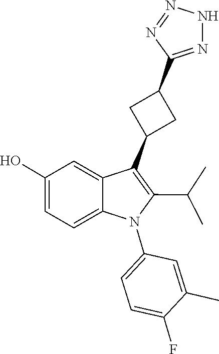 [SMILES: Cc1cc(-n2c(C(C)C)c([C@H]3C[C@@H](c4nn[nH]n4)C3)c3cc(O)ccc32)ccc1F] | 217 |
| 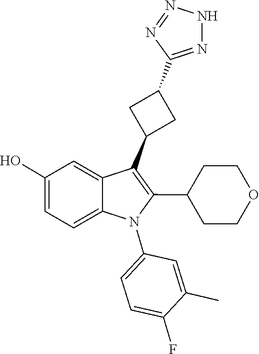 [SMILES: Cc1cc(-n2c(C3CCOCC3)c([C@H]3C[C@H](c4nn[nH]n4)C3)c3cc(O)ccc32)ccc1F] | 218 |
| 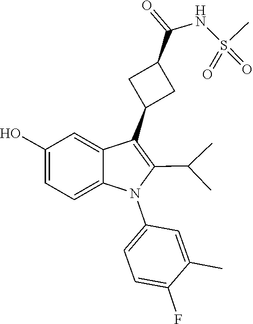 [SMILES: Cc1cc(-n2c(C(C)C)c([C@H]3C[C@@H](C(=O)NS(C)(=O)=O)C3)c3cc(O)ccc32)ccc1F] | 219 |
| 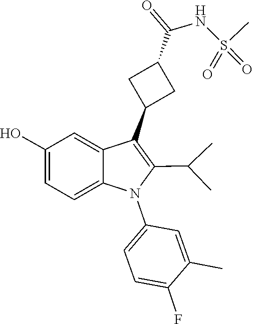 [SMILES: Cc1cc(-n2c(C(C)C)c([C@H]3C[C@H](C(=O)NS(C)(=O)=O)C3)c3cc(O)ccc32)ccc1F] | 220 |
| 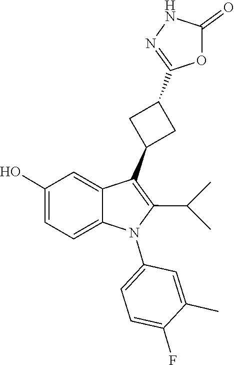 [SMILES: Cc1cc(-n2c(C(C)C)c([C@H]3C[C@H](c4n[nH]c(=O)o4)C3)c3cc(O)ccc32)ccc1F] | 221 |
| 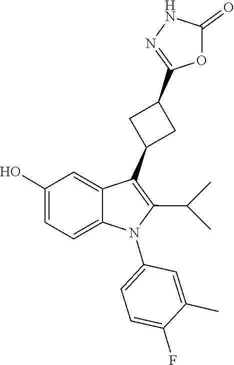 [SMILES: Cc1cc(-n2c(C(C)C)c([C@H]3C[C@@H](c4n[nH]c(=O)o4)C3)c3cc(O)ccc32)ccc1F] | 222 |
| 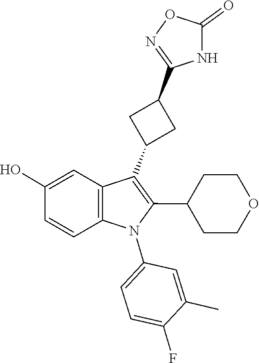 [SMILES: Cc1cc(-n2c(C3CCOCC3)c([C@H]3C[C@H](c4noc(=O)[nH]4)C3)c3cc(O)ccc32)ccc1F] | 223 |
| 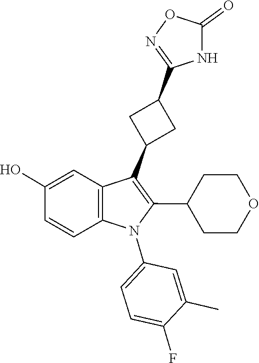 [SMILES: Cc1cc(-n2c(C3CCOCC3)c([C@H]3C[C@@H](c4noc(=O)[nH]4)C3)c3cc(O)ccc32)ccc1F] | 224 |
| 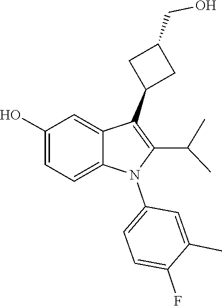 [SMILES: Cc1cc(-n2c(C(C)C)c([C@H]3C[C@H](CO)C3)c3cc(O)ccc32)ccc1F] | 225 |
| 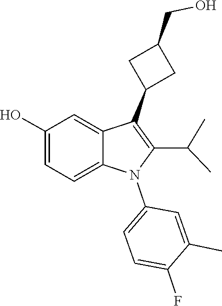 [SMILES: Cc1cc(-n2c(C(C)C)c([C@H]3C[C@@H](CO)C3)c3cc(O)ccc32)ccc1F] | 226 |
| 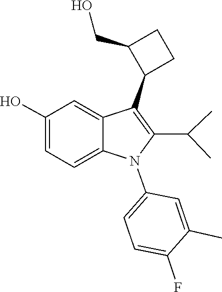 [SMILES: Cc1cc(-n2c(C(C)C)c([C@@H]3CC[C@@H]3CO)c3cc(O)ccc32)ccc1F] | 227 |

a tautomer thereof, a deuterated derivative of the compound or tautomer, or a pharmaceutically acceptable salt of any of the foregoing.

Some embodiments of the disclosure include derivatives of Compounds 1-227 or compounds of Formulae (I), (IIa)-(IIc), (III), (IV), (Va)-(Vc), (VIa)-(VIc), and (VIIa)-(VIIe) or tautomers thereof. In some embodiments, the derivatives are silicon derivatives in which at least one carbon atom in a compound selected from Compounds 1-227 and compounds of Formulae (I), (IIa)-(IIc), (III), (IV), (Va)-(Vc), (VIa)-(VIc), and (VIIa)-(VIIe) has been replaced by silicon. In some embodiments, the derivatives are boron derivatives, in which at least one carbon atom in a compound selected from Compounds 1-227, compounds of Formulae (I), (IIa)-(IIc), (III), (IV), (Va)-(Vc), (VIa)-(VIc), and (VIIa)-(VIIe), and tautomers thereof has been replaced by boron. In other embodiments, the derivatives are phosphate derivatives, in which at least one carbon atom in a compound selected from Compounds 1-227, compounds of Formulae (I), (IIa)-(IIc), (III), (IV), (Va)-(Vc), (VIa)-(VIc), and (VIIa)-(VIIe), and tautomers thereof has been replaced by phosphorus. Because the general properties of silicon, boron, and phosphorus are similar to those of carbon, replacement of carbon by silicon, boron, or phosphorus can result in compounds with similar biological activity to a carbon containing original compound.

In some embodiments, the derivative is a silicon derivative in which one carbon atom in a compound selected from Compounds 1-227, compounds of Formulae (I), (IIa)-(IIc), (III), (IV), (Va)-(Vc), (VIa)-(VIc), and (VIIa)-(VIIe), and tautomers thereof has been replaced by silicon. In other embodiments, two carbon atoms have been replaced by silicon. The carbon replaced by silicon may be a non-aromatic carbon. In some embodiments a quaternary carbon atom of a tert-butyl moiety may be replaced by silicon. In some embodiments, the silicon derivatives of the disclosure may include one or more hydrogen atoms replaced by deuterium. For example, one or more hydrogens of a tert-butyl moiety in which the carbon has been replaced by silicon, may be replaced by deuterium. In other embodiments, a silicon derivative of a compound selected from Compounds 1-227, compounds of Formulae (I), (IIa)-(IIc), (III), (IV), (Va)-(Vc), (VIa)-(VIc), and (VIIa)-(VIIe), and tautomers thereof may have silicon incorporated into a heterocycle ring.

In some embodiments, examples of silicon derivatives of Compounds 1-227 or compounds of Formulae (I), (IIa)-(IIc), (III), (IV), (Va)-(Vc), (VIa)-(VIc), and (VIIa)-(VIIe) include the following compounds:

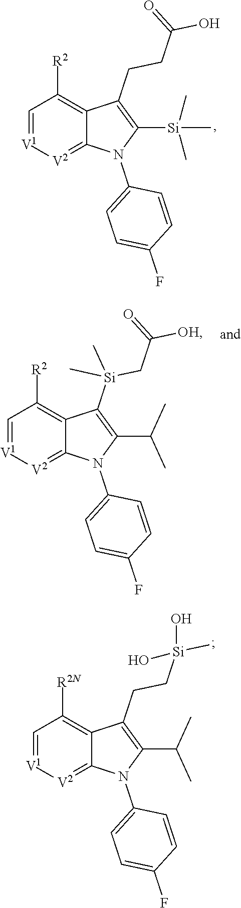

wherein the variables not specifically defined are as defined in any one of Formulae (I), (IIa)-(IIc), (III), (IV), (Va)-(Vc), (VIa)-(VIc), and (VIIa)-(VIIe).

In some embodiments, examples of silicon derivatives of Compounds 1-227 or compounds of Formulae (I), (IIa)-(IIc), (III), (IV), (Va)-(Vc), (VIa)-(VIc), and (VIIa)-(VIIe) include the following compounds:

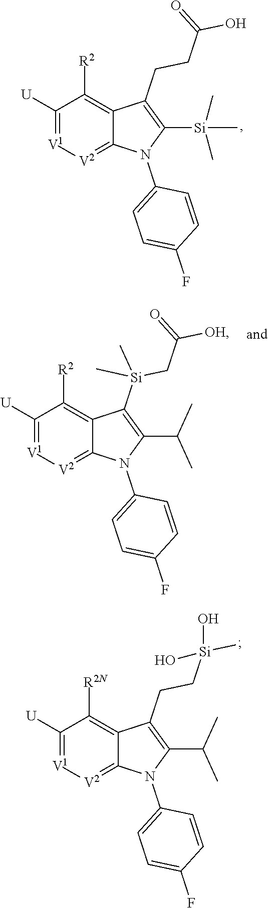

wherein the variables not specifically defined are as defined in any one of Formulae (I), (IIa)-(IIc), (III), (IV), (Va)-(Vc), (VIa)-(VIc), and (VIIa)-(VIIe).

In some embodiments, examples of boron derivatives of Compounds 1-227 or compounds of Formulae (I), (IIa)-(IIc), (III), (IV), (Va)-(Vc), (VIa)-(VIc), and (VIIa)-(VIIe) include the following compounds:

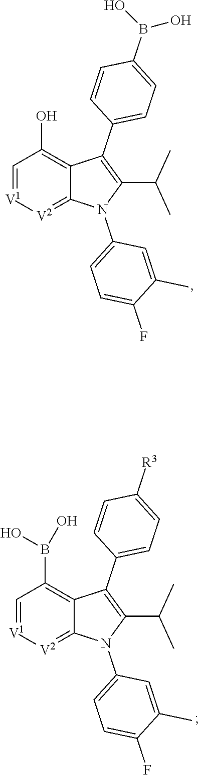

wherein the variables not specifically defined are as defined in any one of Formulae (I), (IIa)-(IIc), (III), (IV), (Va)-(Vc), (VIa)-(VIc), and (VIIa)-(VIIe).

In some embodiments, examples of boron derivatives of Compounds 1-227 or compounds of Formulae (I), (IIa)-(IIc), (III), (IV), (Va)-(Vc), (VIa)-(VIc), and (VIIa)-(VIIe) include the following compounds:

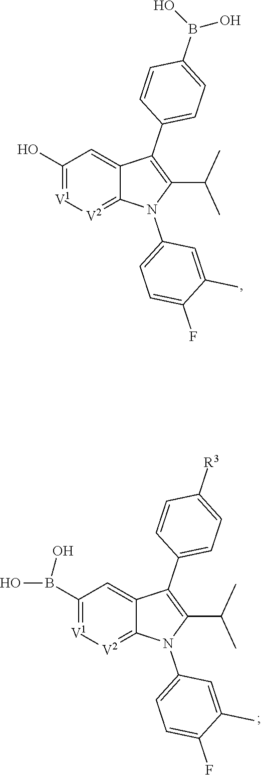

wherein the variables not specifically defined are as defined in any one of Formulae (I), (IIa)-(IIc), (III), (IV), (Va)-(Vc), (VIa)-(VIc), and (VIIa)-(VIIe).

In some embodiments, examples of phosphate derivatives of Compounds 1-227 or compounds of Formulae (I), (IIa)-(IIc), (III), (IV), (Va)-(Vc), (VIa)-(VIc), and (VIIa)-(VIIe) include the following compounds:

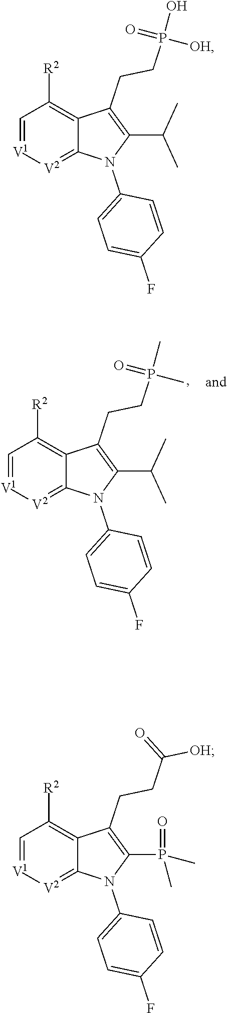

wherein the variables not specifically defined are as defined in any one of Formulae (I), (IIa)-(IIc), (III), (IV), (Va)-(Vc), (VIa)-(VIc), and (VIIa)-(VIIe).

In some embodiments, examples of phosphate derivatives of Compounds 1-227 or compounds of Formulae (I), (IIa)-(IIc), (III), (IV), (Va)-(Vc), (VIa)-(VIc), and (VIIa)-(VIIe) include the following compounds:

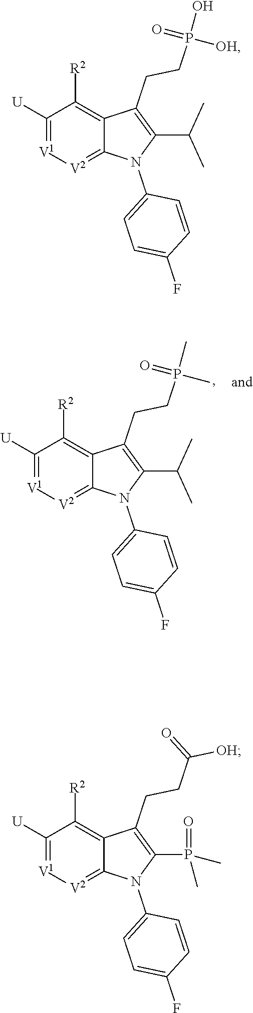

wherein the variables not specifically defined are as defined in any one of Formulae (I), (IIa)-(IIc), (III), (IV), (Va)-(Vc), (VIa)-(VIc), and (VIIa)-(VIIe).

Another aspect of the disclosure provides pharmaceutical compositions comprising a compound selected from compounds according to any of Formulae (I), (IIa)-(IIc), (III), (IV), (Va)-(Vc), (VIa)-(VIc), and (VIIa)-(VIIe), Compounds 1-227, tautomers of those compounds, deuterated derivatives of those compounds and tautomers, and pharmaceutically acceptable salts of any of the foregoing. In some embodiments, the pharmaceutical composition comprising at least one compound chosen from Formulae (I), (IIa)-(IIc), (III), (IV), (Va)-(Vc), (VIa)-(VIc), and (VIIa)-(VIIe) and Compounds 1-227, tautomers of those compounds, deuterated derivatives of those compounds and tautomers, and pharmaceutically acceptable salts of any of the foregoing is administered to a patient in need thereof.

A pharmaceutical composition may further comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, lubricants.

It will also be appreciated that a pharmaceutical composition of this disclosure can be employed in combination therapies; that is, the pharmaceutical compositions described herein can further include at least one other active agent. Alternatively, a pharmaceutical composition comprising at least one compound of Formulae (I), (IIa)-(IIc), (III), (IV), (Va)-(Vc), (VIa)-(VIc), and (VIIa)-(VIIe), tautomers of those compounds, deuterated derivatives of those compounds and tautomers, and pharmaceutically acceptable salts of any of the foregoing can be administered as a separate composition concurrently with, prior to, or subsequent to, a composition comprising at least one additional active agent. In some embodiments, a pharmaceutical composition comprising at least one compound selected from Compounds 1-227 tautomers of those compounds, deuterated derivatives of those compounds and tautomers, and pharmaceutically acceptable salts of any of the foregoing can be administered as a separate composition concurrently with, prior to, or subsequent to, a composition comprising at least one additional active agent.

In some embodiments, a compound of Formula (I), (IIa)-(IIc), (III), (IV), (Va)-(Vc), (VIa)-(VIc), and (VIIa)-(VIIe), tautomers of those compounds, deuterated derivatives of those compounds and tautomers, and pharmaceutically acceptable salts of any of the foregoing, is combined with at least one additional active agent for simultaneous, separate, or sequential use in the treatment of AATD. In some embodiments, when the use is simultaneous, the compound of Formula (I), (IIa)-(IIc), (III), (IV), (Va)-(Vc), (VIa)-(VIc), and (VIIa)-(VIIe), tautomers of those compounds, deuterated derivatives of those compounds and tautomers, and pharmaceutically acceptable salts of any of the foregoing, and the at least one additional active agent are in separate pharmaceutical compostions. In some embodiments, when the use is simultaneous, the compound of Formula (I), (IIa)-(IIc), (III), (IV), (Va)-(Vc), (VIa)-(VIc), and (VIIa)-(VIIe), tautomers of those compounds, deuterated derivatives of those compounds and tautomers, and pharmaceutically acceptable salts of any of the foregoing, and the at least one additional active agent are together in the same pharmaceutical composition. In some embodiments, the compound is a compound selected from Compounds 1-227, tautomers of those compounds, deuterated derivatives of those compounds and tautomers, and pharmaceutically acceptable salts of any of the foregoing.

In some embodiments, a compound of Formula (I), (IIa)-(IIc), (III), (IV), (Va)-(Vc), (VIa)-(VIc), and (VIIa)-(VIIe), tautomers of those compounds, deuterated derivatives of those compounds and tautomers, and pharmaceutically acceptable salts of any of the foregoing, is provided for use in a method of treating AATD, wherein the method comprises co-administering the compound and an additional active agent. In some embodiments, the compound and the additional active agent are co-administered in the same pharmaceutical composition. In some embodiments, the compound and the additional active agent are co-administered in separate pharmaceutical compositions. In some embodiments, the compound and the additional active agent are co-administered simultaneously. In some embodiments, the compound and the additional active agent are co-administered sequentially. In some embodiments, the compound is selected from Compounds 1-227, tautomers of those compounds, deuterated derivatives of those compounds and tautomers, and pharmaceutically acceptable salts of any of the foregoing.

In some embodiments, a combination of a compound of Formula (I), (IIa)-(IIc), (III), (IV), (Va)-(Vc), (VIa)-(VIc), and (VIIa)-(VIIe), tautomers of those compounds, deuterated derivatives of those compounds and tautomers, and pharmaceutically acceptable salts of any of the foregoing, and an additional active agent, is provided for use in a method of treating AATD. In some embodiments, the compound and the additional active agent are co-administered in the same pharmaceutical composition. In some embodiments, the compound and the additional active agent are co-administered in separate pharmaceutical compositions. In some embodiments, the compound and the additional active agent are co-administered simultaneously. In some embodiments, the compound and the additional active agent are co-administered sequentially. In some embodiments, the compound is selected from Compounds 1-227, tautomers of those compounds, deuterated derivatives of those compounds and tautomers, and pharmaceutically acceptable salts of any of the foregoing.

In some embodiments, an additional active agent is provided for use in a method of treating AATD, wherein the method comprises co-administrating the additional active agent and a compound of Formula (I), (IIa)-(IIc), (III), (IV), (Va)-(Vc), (VIa)-(VIc), and (VIIa)-(VIIe), tautomers of those compounds, deuterated derivatives of those compounds and tautomers, and pharmaceutically acceptable salts of any of the foregoing. In some embodiments, the compound and the additional active agent are co-administered in the same pharmaceutical composition. In some embodiments, the compound and the additional active agent are co-administered in separate pharmaceutical compositions. In some embodiments, the compound and the additional active agent are co-administered simultaneously. In some embodiments, the compound and the additional active agent are co-administered sequentially. In some embodiments, the compound is selected from Compounds 1-227, tautomers of those compounds, deuterated derivatives of those compounds and tautomers, and pharmaceutically acceptable salts of any of the foregoing.

In some embodiments, a compound of Formula (I), (IIa)-(IIc), (III), (IV), (Va)-(Vc), (VIa)-(VIc), and (VIIa)-(VIIe), tautomers of those compounds, deuterated derivatives of those compounds and tautomers, and pharmaceutically acceptable salts of any of the foregoing, is provided for use in a method of treating AATD, wherein the compound is prepared for administration in combination with an additional active agent. In some embodiments, the compound and the additional active agent are prepared for administration in the same pharmaceutical composition. In some embodiments, the compound and the additional active agent are prepared for administration in separate pharmaceutical compositions. In some embodiments, the compound and the additional active agent are prepared for simultaneous administration. In some embodiments, the compound and the additional active agent are prepared for sequential administration. In some embodiments, the compound is selected from Compounds 1-227, tautomers of those compounds, deuterated derivatives of those compounds and tautomers, and pharmaceutically acceptable salts of any of the foregoing.

In some embodiments, a combination of a compound of Formula (I), (IIa)-(IIc), (III), (IV), (Va)-(Vc), (VIa)-(VIc), and (VIIa)-(VIIe), tautomers of those compounds, deuterated derivatives of those compounds and tautomers, and pharmaceutically acceptable salts of any of the foregoing, and an additional active agent, is provided for use in a method of treating AATD. In some embodiments, the compound and the additional active agent are prepared for administration in the same pharmaceutical composition. In some embodiments, the compound and the additional active agent are prepared for administration in separate pharmaceutical compositions. In some embodiments, the compound and the additional active agent are prepared for simultaneous administration. In some embodiments, the compound and the additional active agent are prepared for sequential administration. In some embodiments, the compound is selected from Compounds 1-227, tautomers of those compounds, deuterated derivatives of those compounds and tautomers, and pharmaceutically acceptable salts of any of the foregoing.

In some embodiments, an additional active agent is provided for use in a method of treating AATD, wherein the additional active agent is prepared for administration in combination with a compound of Formula (I), (IIa)-(IIc), (III), (IV), (Va)-(Vc), (VIa)-(VIc), and (VIIa)-(VIIe), tautomers of those compounds, deuterated derivatives of those compounds and tautomers, and pharmaceutically acceptable salts of any of the foregoing. In some embodiments, the compound and the additional active agent are prepared for administration in the same pharmaceutical composition. In some embodiments, the compound and the additional active agent are prepared for administration in separate pharmaceutical compositions. In some embodiments, the compound and the additional active agent are prepared for simultaneous administration. In some embodiments, the compound and the additional active agent are prepared for sequential administration. In some embodiments, the compound is selected from Compounds 1-227, tautomers of those compounds, deuterated derivatives of those compounds and tautomers, and pharmaceutically acceptable salts of any of the foregoing.

In some embodiments, the additional active agent is selected the group consisting of alpha-1 antitrypsin protein (AAT) from the blood plasma of healthy human donors and recombinant AAT. In some embodiments, the additional active agent is alpha-1 antitrypsin protein (AAT) from the blood plasma of healthy human donors. In some embodiments, the additional active agent is alpha-1 antitrypsin protein (AAT) from the blood plasma of healthy human donors.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

In another aspect of the disclosure, the compounds and the pharmaceutical compositions, described herein, are used to treat AATD. In some embodiments, the subject in need of treatment with the compounds and compositions of the disclosure carries the ZZ mutation. In some embodiments, the subject in need of treatment with the compounds and compositions of the disclosure carries the SZ mutation.

In some embodiments, the methods of the disclosure comprise administering to a patient in need thereof a compound chosen from any of the compounds of Formulae (I), (IIa)-(IIc), (III), (IV), (Va)-(Vc), (VIa)-(VIc), and (VIIa)-(VIIe), tautomers of those compounds, deuterated derivatives of those compounds and tautomers, and pharmaceutically acceptable salts of any of the foregoing. In some embodiments, the compound of Formula (I) is selected from Compounds 1-227, tautomers of those compounds, deuterated derivatives of those compounds and tautomers, and pharmaceutically acceptable salts of any of the foregoing. In some embodiments, said patient in need thereof has a Z mutation in the alpha-1 antitrypsin gene. In some embodiments said patient in need thereof is homozygous for the Z-mutation in the alpha-1 antitrypsin gene.

Another aspect of the disclosure provides methods of modulating alpha-1 antitrypsin activity comprising the step of contacting said alpha-1-antitrypsin with at least one compound of Formulae (I), (IIa)-(IIc), (III), (IV), (Va)-(Vc), (VIa)-(VIc), and (VIIa)-(VIIe), tautomers of those compounds, deuterated derivatives of those compounds and tautomers, and pharmaceutically acceptable salts of any of the foregoing. In some embodiments, the methods of modulating alpha-1 antitrypsin activity comprising the step of contacting said alpha-1-antitrypsin with at least one compound selected from Compounds 1-227, tautomers of those compounds, deuterated derivatives of those compounds and tautomers, and pharmaceutically acceptable salts of any of the foregoing.

In some embodiments, the methods of modulating alpha-1 antitrypsin activity take place in vivo. In some embodiments, the methods of modulating alpha-1 antitrypsin activity take place ex vivo and said alpha-1-antitrypsin is from a biological sample obtained from a human subject. In some embodiments, the methods of modulating AAT take place in vitro and said alpha-1-antitrypsin is from a biological sample obtained from a human subject. In some embodiments, the biological sample is a blood sample. In some embodiments, the biological sample is a sample taken from a liver biopsy.

### III. PREPARATION OF COMPOUNDS

All the generic, subgeneric, and specific compound formulae disclosed herein are considered part of the disclosure.

A. Compounds of Formula I

The compounds of the disclosure may be made according to standard chemical practices or as described herein. Throughout the following synthetic schemes and in the descriptions for preparing compounds of Formulae (I), (IIa)-(IIc), (III), (IV), (Va)-(Vc), (VIa)-(VIc), and (VIIa)-(VIIe), Compounds 1-227, tautomers of those compounds, deuterated derivatives of those compounds and tautomers, and pharmaceutically acceptable salts of any of the foregoing, the following abbreviations are used:

### ABBREVIATIONS

BrettPhos Pd G4=dicyclohexyl-[3,6-dimethoxy-2-[2,4,6-tri(propan-2-yl)phenyl]phenyl]phosphane; methanesulfonic acid; N-methyl-2-phenylaniline; palladium
DIPEA=N,N-Diisopropylethylamine or N-ethyl-N-isopropyl-propan-2-amine
DMA=dimethyl acetamide
DMAP=dimethylamino pyridine
DME=dimethoxyethane
DMF=dimethylformamide
DMSO=dimethyl sulfoxide
EtOH=ethanol
EtOAc=ethyl acetate
HATU=[dimethylamino(triazolo[4,5-b]pyridin-3-yloxy)methylene]-dimethyl-ammonium (Phosphorus Hexafluoride Ion)
MeOH=methanol
MP-TMT scavenger resin=a macroporous polystyrene-bound trimercaptotriazine, a resin bound equivalent of 2,4,6-trimercaptotriazine (TMT).
MTBE=Methyl tert-butyl ether
NMM=N-methyl morpholine
NMP=N-methyl pyrrolidine
Pd(dppf)₂Cl₂=[1,1′-Bis(diphenylphosphino)ferrocene]dichloropalladium(II)
PdCl₂=palladium(II) dichloride
PdCl₂(PPh₃)₂=Bis(triphenylphosphine)palladium(II) dichloride
SFC=super critical fluid chromatography
SPhos Pd G3=(2-Dicyclohexylphosphino-2′,6′-dimethoxybiphenyl) [2-(2′-amino-1,1′-biphenyl)]palladium(II) methanesulfonate
TBAF=Tetrabutylammonium fluoride
tBuXPhos Pd G1=Chloro[2-(di-tert-butylphosphino)-2′,4′,6′-triisopropyl-1,1′-biphenyl][2-(2-aminoethyl)phenyl)]palladium(II) or t-BuXPhos palladium(II) phenethylamine chloride
tBuXPhos Pd G3=[(2-Di-tert-butylphosphino-2′,4′,6′-triisopropyl-1,1′-biphenyl)-2-(2′-amino-1,1′-biphenyl)] palladium(II) methanesulfonate
tBuXPhos Pd G4=ditert-butyl-[2-(2,4,6-triisopropylphenyl)phenyl]phosphane; dichloromethane; methanesulfonate; N-methyl-2-phenyl-aniline palladium (II)
TFA=trifluoroacetic acid
THE=tetrahydrofuran
XPhos Pd G1=(2-Dicyclohexylphosphino-2′,4′,6′-triisopropyl-1,1′-biphenyl)[2-(2-aminoethyl)phenyl)]palladium(II) chloride or (XPhos) palladium(II) phenethylamine chloride.

In some embodiments, processes for preparing compounds of Formula (I), tautomers thereof, deuterated derivatives of those compounds and tautomers, or pharmaceutically acceptable salts of any of the foregoing, comprise reacting a compound of Formula (I), tautomer, deuterated derivative, or pharmaceutically acceptable salt with a deprotection reagent as depicted in Schemes 1 through 11 below (wherein all variables are as defined for Formula (I) above):

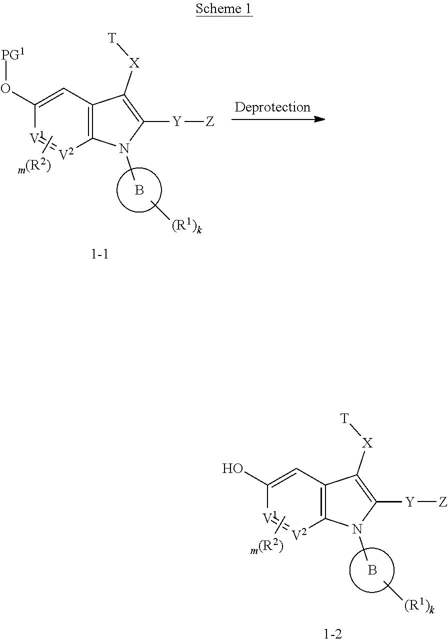

Scheme 1 shows methods for the preparation of a compound of Formula (I). PG¹ is an alcohol protecting group such as Benzyl (Bn), Methoxymethyl (MOM), or Methyl. In some examples, where PG¹ is a benzyl group, a compound of formula 1-2 may be prepared by hydrogenolysis of a compound of formula 1-1 using a palladium on carbon catalyst, under an atmosphere of hydrogen. The reaction may be performed at elevated pressure. A solvent such as Methanol, EtOH or EtOAc may be used. Where PG¹ is a group such as MOM, a compound of Formula (I) may be prepared by treatment with acid such as HCl. In examples where PG¹ is a methyl group, the group may be removed by treatment with AlCl3 in the presence of octanethiol. In some examples, a reagent such as BBr₃ may be used. Any other standard method suitable for the removal of an alcohol group may be used to prepare compound of formula 1-2 from compounds of formula 1-1.

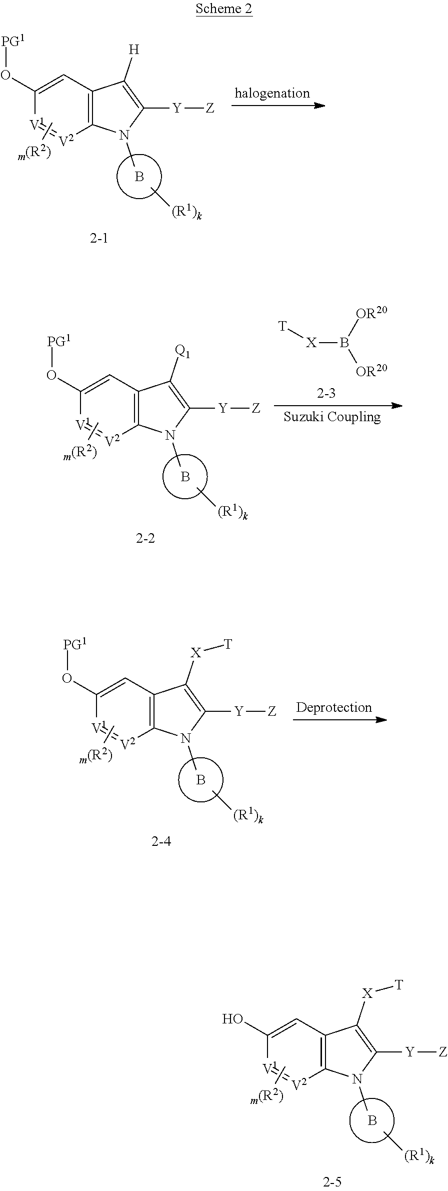

Scheme 2 shows methods for the preparation of a compound of formula 2-5. Q¹ is a halogen such as Br, I or Cl. Compounds of formula 2-3 are boronic acids or esters with R²⁰ an alkyl group (Me), or hydrogen. All other variables are as defined above. Compounds of formula 2-1 may be transformed into compounds of formula 2-2 using any suitable method for the halogenation reaction. For example, N-iodosuccinimide (NIS) or N-bromosuccinimide (NBS) in a solvent such as dichloromethane may be used. A compound of formula 2-4 from 2-2 and 2-3 using standard Suzuki coupling conditions. In some examples, Suzuki coupling conditions may involve a catalyst such as Pd(dppf)Cl₂ and a base such as Na₂CO₃. In some examples, a catalyst such as Pd₂(dba)₃ in the presence of a ligand such as XPhos may be used. A solvent such as DMF or DME may be used. The reaction is performed in the presence of additional heat (e.g. 90° C.). A compound of formula 2-5 may be prepared from 2-4 using any suitable method for the removal of an alcohol protecting group.

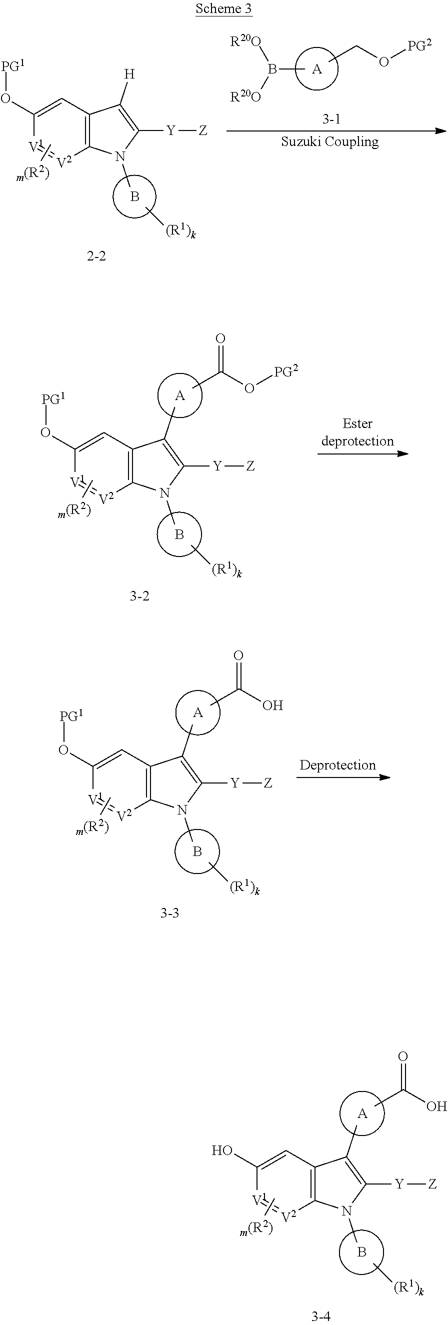

Processes for the preparation of compounds of formula 3-4 are shown in Scheme 3. PG² is any suitable carboxylic acid protecting group. For example, PG² may be Me, Et, Benzyl or tert-Butyl. All other variables are defined as above. Compounds of formula 3-2 may be prepared from compounds of formula 3-1 using any suitable method for Suzuki coupling. For example, Pd(dppf)Cl₂ in the presence of Na₂CO₃ may be used. Compounds of formula 3-3 may be prepared from compounds of formula 3-2 using any suitable method for the removal of a carboxylic acid protecting group. For example, where PG² is a methyl ester, hydrolysis with a base such as LiOH or NaOH in a solvent such as THE and water may be used. Where PG² is a group such as tert-Butyl, treatment with an acid such as TFA or HCl affords compounds of formula 3-3. In some examples, where PG¹ and PG² are both benzyl groups, a compound of formula 3-4 may be prepared directly from a compound of formula 3-2 by hydrogenation.

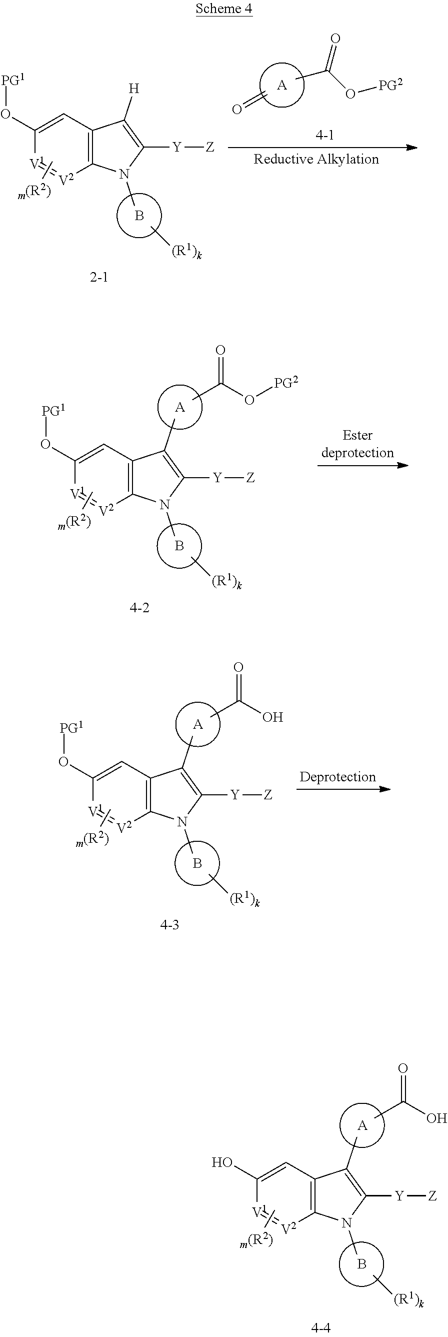

Scheme 4 shows processes for the preparation of compounds of formula 4-4. All variables are defined as above. Compounds of formula 4-2 may be prepared by reductive alkylation between an indole of formula 2-1 and a ketone of formula 4-1. In some examples, reductive alkylation may be performed in the presence of a reagent such as triethyl silane and an acid (such as trifluoroacetic acid or methanesulfonic acid). The reaction may be performed in a solvent such as dichloromethane.

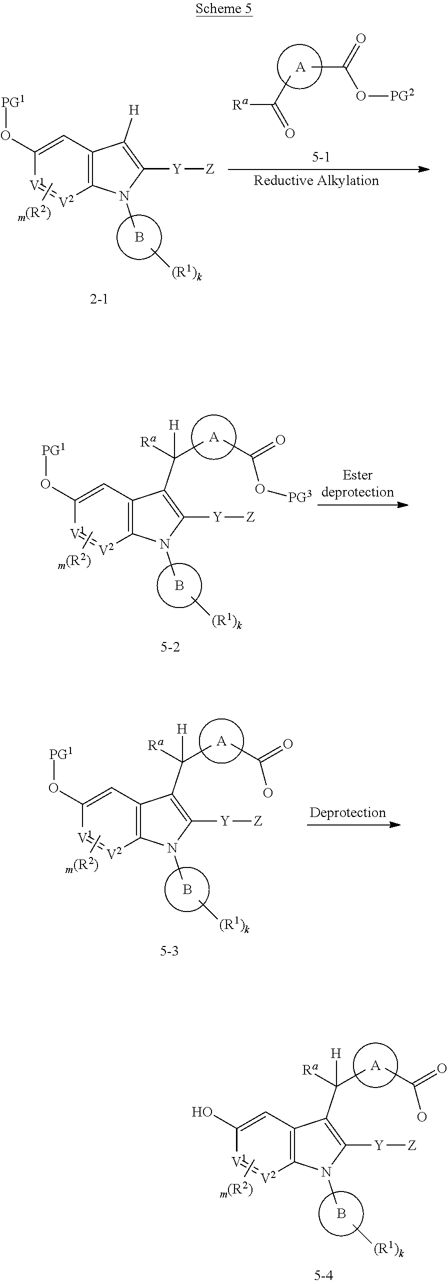

Scheme 5 depicts methods for the preparation of compounds of formula 5-4. All variables are defined as above. Compound of formula 5-2 may be prepared from ketones or aldehydes of formula 5-1 and indoles of formula 2-1 using any suitable conditions for performing a reductive alkylation reaction. In some examples, the reaction may be performed in the presence of triethyl silane and trifluoroacetic acid. A solvent such as dichloromethane may be used. The reaction may be performed in the presence of added heat (e.g. at 40° C.).

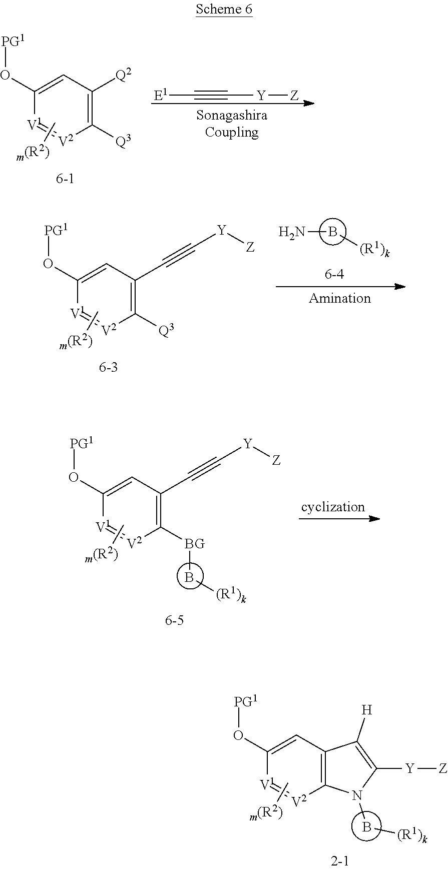

Scheme 6 shows processes for the preparation of indoles of formula 2-1. Q² and Q³ are halogens such as Br, Cl or I. E¹ is hydrogen or SiMe₃. For example, in some processes Q² is iodine and Q³ is bromine. In some examples, compounds of formula 6-3 may be prepared from compound of formula 6-1 and alkynes of formula 6-2 using any suitable conditions for performing a Sonagashira coupling. In some examples, a catalyst such a Pd(PPh₃)₂Cl₂ in the presence of CuI may be used. A base such as triethylamine or diisopropylethylamine may be used. The reaction may be performed in a solvent such as DMF in the presence of added heat. In some examples, where E¹ is SiMe₃, the reaction may be performed in the presence of TBAF. Compounds of formula 6-5 may be prepared from compounds of formula 6-3 by transition metal catalyzed amination with an amine of formula 6-4. Amination may be performed in the presence of a palladium catalyst such as tBuXPhos Pd G3, tBuXPhos Pd G, or any other suitable catalyst for performing Buchwald aminations. A base such as NaOtBu may be used. The reaction may be performed in a solvent such as xylene. The reaction may be performed at room temperature, or in the presence of added heat. In some example, cyclization to compounds of formula 2-1 occurs spontaneously in the amination reaction. In some examples, compounds of formula 2-1 from 6-5 are prepared by treatment with PdCl₂ in a solvent such as MeCN. The reaction may be performed with added heat (e.g. at 50° C.).

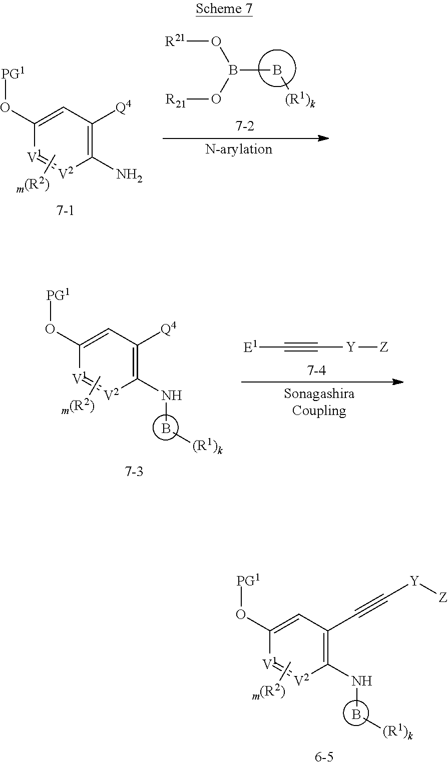

Scheme 7 shows an alternative process for the preparation of a compound of formula 6-5. Q⁴ is a halogen such as Br or I. R²¹ is a hydrogen or an alkyl group such as ethyl. An aniline of formula 7-1 may be arylated with a boronic acid or ester 7-2 using any suitable conditions for N-arylation to give a compound of formula 7-3. In some examples, a Cu(OAc)₂ catalyst may be used. The reaction may be performed in the presence of a base such as K₂CO₃. A solvent such as DMSO may be used. A compound of formula 6-5 may be prepared by Sonagashira coupling of compounds of formula 7-3 with alkynes of formula 7-4 to afford compounds of formula 6-5.

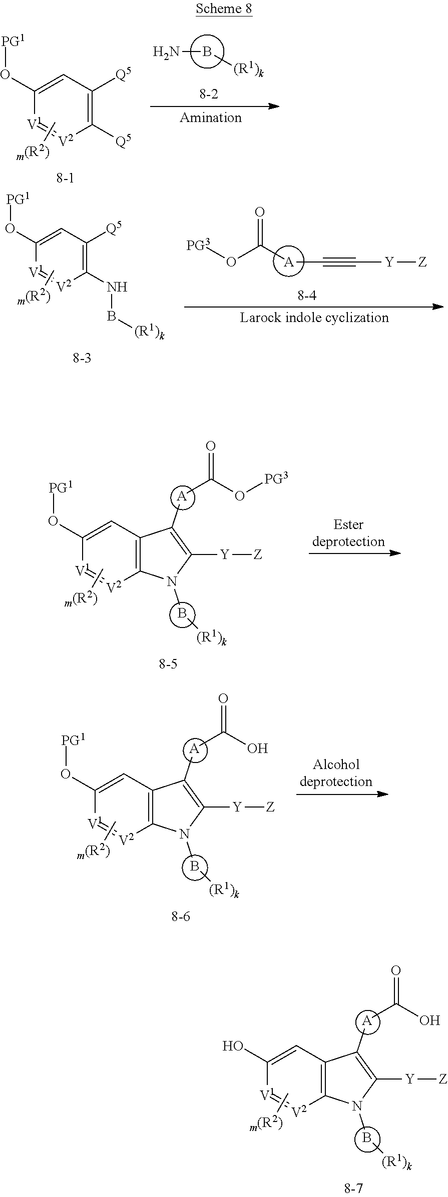

Scheme 8 depicts processes for the preparation of compounds of formula 8-7 from a dihaloaryl of general formula 8-1. Q⁵ is a halogen such as Cl, Br, or I. In some embodiments, group A is an aromatic or heteroaromatic ring. Amination of compound of formula 8-1 with an amine of formula 8-2 affords compounds of formula 8-3. Any suitable method for amination of an aryl halide with an amine may be used. For example, the reaction may be performed in the presence of a catalyst such as Pd(OAc)₂ in the presence of a ligand such as dppf In some examples, the reaction may be performed in the presence of tBuXPhos Pd G1. The reaction may be performed in the presence of a base such as NaOtBu. Indoles of formula 8-5 may be prepared by reaction of compounds of formula 8-3 with disubstituted alkynes of formula 8-4 in the presence of a suitable palladium catalyst. For example, catalysts such as Pd(tBu₃P)₂ or JackiePhos Pd G3 may be used. In some alternative embodiments, Pd(OAc)₂ may be used. The reaction is performed in the presence of a suitable ligand. For example, dicyclohexyl methylamine (cHx)₂NMe may be used. The reaction may be performed in a solvent such as 1,4-dioxane, and in the presence of added heat (e.g. 60° C.).

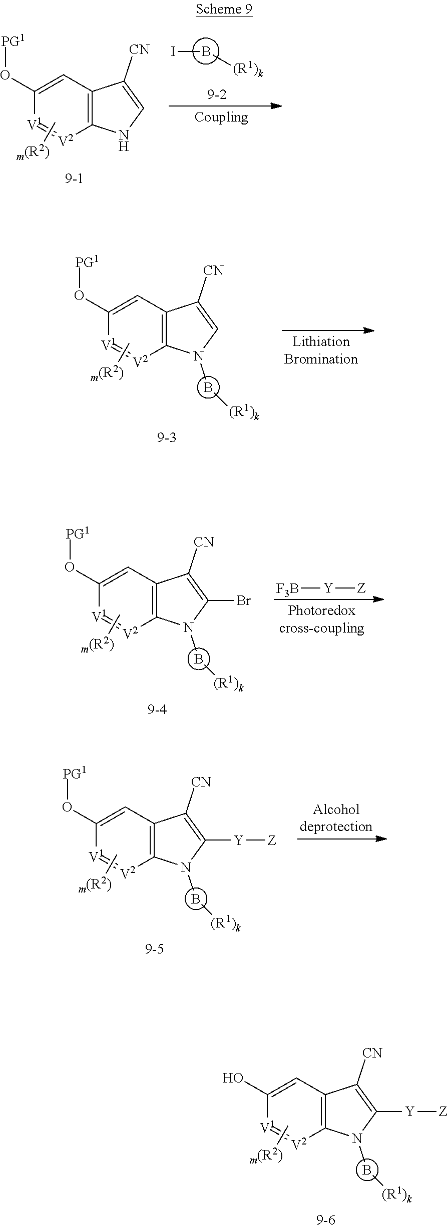

Any suitable conditions for Chan-Lam coupling of a compound of formula 9-1 with an iodide of formula 9-2, as shown in scheme 9, may be used in the preparation of compounds of formula 9-3. Compounds of formula 9-4 may be prepared from compounds of formula 9-3 using any suitable method for bromination of indoles at C2 position. In some embodiments, the reaction is performed in the presence of tert-butyllithium followed by quenching with a source of electrophilic bromide, such as 1,2-dibromotetrachloroethane. sp2-sp3 coupling to afford compounds of formula 9-5 from indoles of formula 9-4 can be carried out using photoredox cross-coupling conditions. For example, using trifluoroborate salts with an iridium based photocatalyst in a flow reactor irradiating with a Vaportech LED 124 Watt lamp @ 450 nM. Compounds of formula 9-6 may be prepared from compounds of formula 9-5 using standard methods for alcohol deprotection.

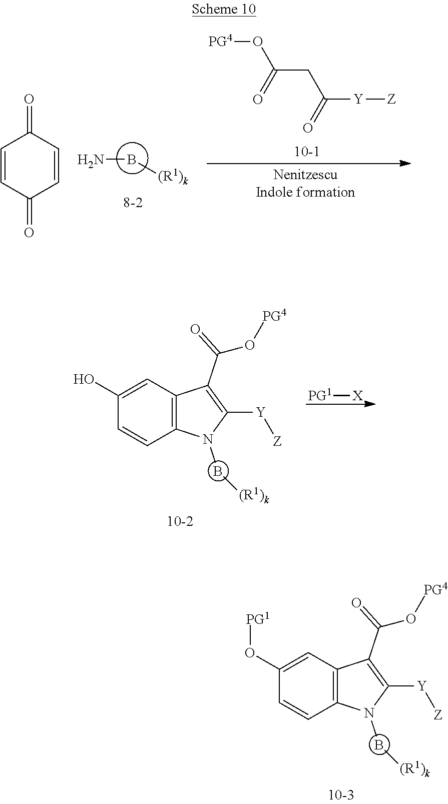

Any suitable conditions for Nenitzescu indole formation of benzoquinone with an amine of formula 8-2 with a keto ester of formula 10-1, as shown in scheme 10, may be used in the preparation of compounds of formula 10-2. In some embodiments, the reaction is performed in the presence of zinc chloride and acetic acid. Compounds of formula 10-3 may be prepared from compounds of formula 10-2 using standard methods for alcohol protection.

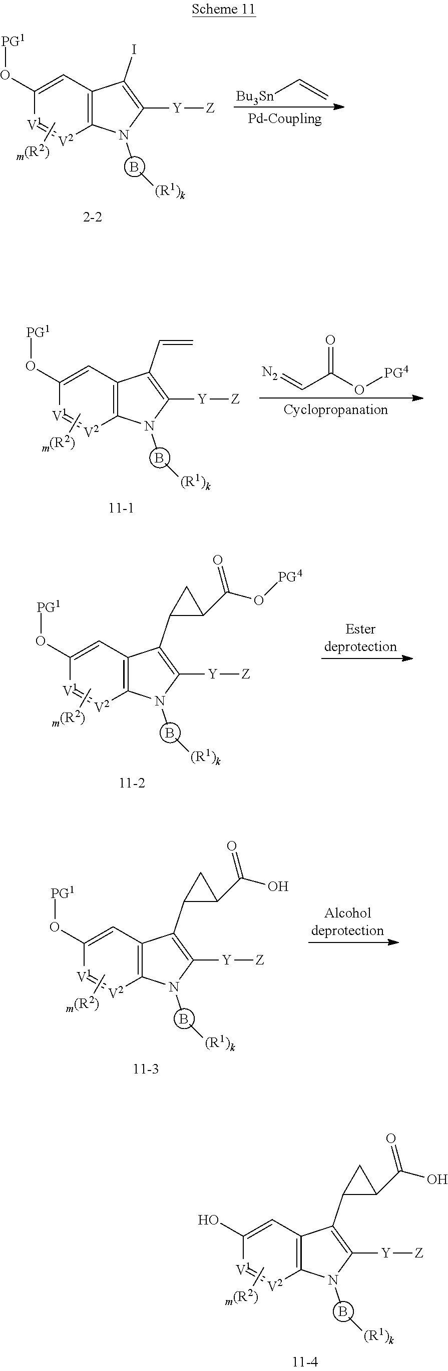

Any suitable conditions for Stille cross-coupling reactions of vinyl-stannanes with an iodide of formula 2-2, as shown in Scheme 11, may be used in the preparation of compounds of formula 11-1. In some embodiments, the reaction is performed in the presence of palladium tetrakis and tetraethylammonium chloride with solvents such as dimethylformamide. Cyclopropanation using reagents such as ethyl 2-diazoacetate in presence of (R,R)-PyBox. The reaction may be performed in a solvent such as toluene, and in the presence of added heat (e.g. 50° C.). Compounds of formula 11-3 may be prepared from compounds of formula 11-2 using previously described standard methods for ester hydrolysis. Compounds of formula 11-4 may be prepared from compounds of formula 11-3 using standard methods for alcohol deprotection.

### EXAMPLES

In order that the disclosure described herein may be more fully understood, the following examples are set forth. It should be understood that these examples are for illustrative purposes only and are not to be construed as limiting this disclosure in any manner.

### Example 1. Synthesis of Compounds

All the specific and generic compounds, the methods for making those compounds, and the intermediates disclosed for making those compounds, are considered to be part of this disclosure.

A. Synthesis of Starting Materials

Preparations of S1-S22 describe synthetic routes to intermediates used in the synthesis of Compound 1-227.

### Preparation of S1-S6

### 5-(benzyloxy)-1-(4-fluorophenyl)-2-(tetrahydro-2H-pyran-4-yl)-1H-indole (S1)

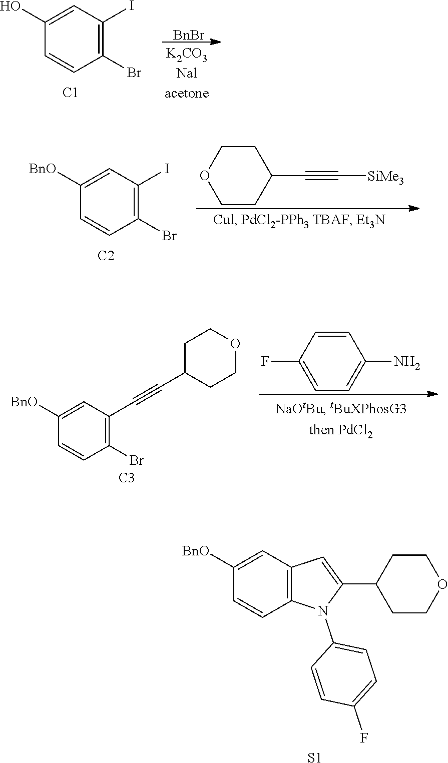

### Step 1. Synthesis of 4-(benzyloxy)-1-bromo-2-iodobenzene (C2)

To a solution of 4-bromo-3-iodo-phenol (88.1 g, 291.9 mmol) in acetone (840 mL) was added K₂CO₃ (48.4 g, 350.3 mmol) and NaI (13.1 g, 87.6 mmol). The resulting suspension was heated to 45-50° C. Benzyl bromide (36.7 mL, 306.5 mmol) was added dropwise and the reaction mixture was heated at 50° C. overnight. The reaction mixture was then cooled to room temperature. The solids were removed by filtration and washed with acetone. The resulting filtrate was concentrated in vacuo, diluted with dichloromethane (400 mL) and washed with 1M NaOH (2×200 mL). The aqueous phases were extracted with dichloromethane (200 mL) and the combined organic layers were dried over Na₂SO₄, filtered and concentrated to afford the 112 g of the desired product. 4-Benzyloxy-1-bromo-2-iodo-benzene (99%). ¹H NMR (300 MHz, Chloroform-d) δ 7.50-7.32 (m, 7H), 6.84 (dd, J=8.8, 2.9 Hz, 1H), 5.02 (s, 2H).

### Step 2. Synthesis of 4-((5-(benzyloxy)-2-bromophenyl)ethynyl)tetrahydro-2H-pyran (C3)

To a solution of 4-benzyloxy-1-bromo-2-iodo-benzene C2 (141.1 g, 344.6 mmol) and trimethyl(2-tetrahydropyran-4-ylethynyl)silane (75.0 g, 407.2 mmol) in triethylamine (900 mL) was added water (13.0 mL, 721.6 mmol) followed by copper iodide (8.0 g, 42.0 mmol) and dichloropalladium; triphenylphosphane (12.0 g, 17.1 mmol). The reaction mixture was purged with nitrogen for 2 minutes and then cooled to 0° C. for 5 minutes. To the mixture was added tetrabutylammonium fluoride (430 mL of 1 M solution in THF, 430.0 mmol). The reaction was stirred at room temperature overnight. The solvents were removed under reduced pressure. The resulting residue was diluted with dichloromethane and filtered through a pad of silica gel. The resulting filtrate was concentrated in vacuo to yield a black oil that crystalized upon standing to afford 320 grams of solid. The solid was diluted again in dichloromethane and filtered through a silica plug using heptane (100%) and then a gradient using (1:9 EtOAc-CH₂Cl₂)/Heptane (0-40%) up until all product comes off. The major homogeneous fractions were concentrated in vacuo and dried under vacuum to give a solid that was triturated with heptanes and filtered. After drying, 81.6 g of a beige solid was obtained. Mother liquor was condensed and was repurified by MPLC—0-15% EtOAc/Heptane on an 880 g silica gel column; Pure fractions gave an oil that crystalized upon standing to afford an additional 49.6 g of desired product. 4-((5-(Benzyloxy)-2-bromophenyl)ethynyl)tetrahydro-2H-pyran (95%). ¹H NMR (300 MHz, Chloroform-d) δ 7.38-7.04 (m, 6H), 6.88 (d, J=3.0 Hz, 1H), 6.59 (dd, J=8.9, 3.1 Hz, 1H), 4.83 (s, 2H), 3.81 (m, 2H), 3.41 (m, 2H), 2.75 (dt, J=7.8, 3.7 Hz, 1H), 1.94-1.42 (m, 4H). ESI-MS m/z calc. 370.06, found 372.36 (M+H)⁺.

### Step 3. 5-(benzyloxy)-1-(4-fluorophenyl)-2-(tetrahydro-2H-pyran-4-yl)-1H-indole (S1)

To a mixture of 4-((5-(benzyloxy)-2-bromophenyl)ethynyl)tetrahydro-2H-pyran C₃ (3.3 g, 8.1 mmol), 4-fluoroaniline (1.0 g, 9.0 mmol) and tBuXxPhos Pd G3 (0.34 g, 0.43 mmol) in dioxane (30 mL) was added sodium tert-butoxide (8.5 mL of 2 M solution, 17.0 mmol). The resulting mixture was stirred for 1 h at 50° C. After cooling to room temperature, the mixture was diluted with CH₂Cl₂, filtered through a pad of celite and filtrate concentrated in vacuo. The residue was purified by silica gel chromatography (80 g ISCO cartridge) eluting with 0-10% EtOAc/CH₂Cl₂ gradient to afford 4-benzyloxy-N-(4-fluorophenyl)-2-(2-tetrahydropyran-4-ylethynyl)aniline that was used without further purification. To a solution of 4-benzyloxy-N-(4-fluorophenyl)-2-(2-tetrahydropyran-4-ylethynyl)aniline in CH₃CN (30 mL) was added PdCl₂ (0.20 g, 1.13 mmol). The reaction mixture was heated at 50° C. After reaction went to completion, the mixture was concentrated in vacuo. The resulting residue was purified by silica gel chromatography (80 g ISCO column) eluting with 0-30%0 CH₂Cl₂/heptanes to afford 1.2 g of product. 5-Benzyloxy-1-(4-fluorophenyl)-2-tetrahydropyran-4-yl-indole (370). ¹H NMR (400 MHz, Chloroform-d) δ 7.50 (d, J=7.0 Hz, 2H), 7.45-7.22 (m, 7H), 7.21-7.11 (i, 1H), 6.96-6.81 (i, 2H), 6.39 (d, J=0.9 Hz, 1H), 5.14 (s, 2H), 4.08-3.92 (1, 2H), 3.35 (td, J=11.8, 2.1 Hz, 2H), 2.79 (ddd, J=11.6, 7.6, 3.8 Hz, 1H), 1.94-1.64 (in, 4H). ESI-MS m/z calc. 401.18, found 402.0 (M+H)⁺.

Compounds S2-S6 (Table 1) were made by a similar method to S1, substituting the appropriate aniline into the Buchwald amination reaction.

TABLE 1
Structure and physicochemical data for intermediates S2-S6

| Intermediate | Structure | Aniline | ¹H NMR; LCMS m/z [M + H]⁺ |
|---|---|---|---|
| S2 | 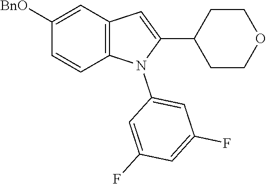 [SMILES: Fc1cc(F)cc(-n2c(C3CCOCC3)cc3cc(OCc4ccccc4)ccc32)c1] | 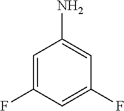 | ¹H NMR (400 MHz, Chloroform-d) δ 7.56-7.30 (m, 6H), 7.17 (d, J = 2.3 Hz, 1H), 7.07-6.83 (m, 5H), 6.42 (d, J = 0.8 Hz, 1H), 5.14 (s, 2H), 4.01 (ddd, J = 11.7, 4.3, 1.8 Hz, 2H), 3.41 (td, J = 11.8, 2.4 Hz, 2H), 2.88 (tt, J = 11.5, 4.0 Hz, 1H), 1.99-1.68 (m, 4H), 1.60 (s, 1H). LCMS m/z 420.0 [M + H]⁺. |
| S3 | 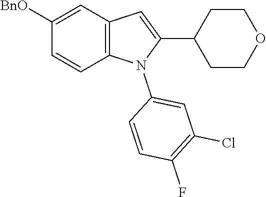 [SMILES: Fc1ccc(-n2c(C3CCOCC3)cc3cc(OCc4ccccc4)ccc32)cc1Cl] | 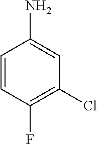 | ¹H NMR (400 MHz, Chloroform-d) δ 7.54-7.33 (m, 7H), 7.27-7.23 (m, 1H), 7.16 (dd, J = 2.2, 0.7 Hz, 1H), 6.95- 6.84 (m, 2H), 6.39 (s, 1H), 5.14 (s, 2H), 4.06-3.96 (m, 2H), 3.38 (td, J = 11.8, 2.2 Hz, 2H), 2.78 (ddd, J = 11.6, 7.6, 3.9 Hz, 1H), 1.94-1.68 (m, 5H). LCMS m/z 436.4 [M + H]⁺. |
| S4 | 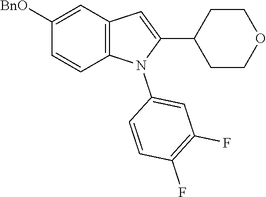 [SMILES: Fc1ccc(-n2c(C3CCOCC3)cc3cc(OCc4ccccc4)ccc32)cc1F] | 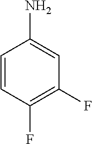 | ¹H NMR (400 MHz, DMSO-d6) δ 7.74-7.62 (m, 2H), 7.50-7.43 (m, 2H), 7.39-7.24 (m, 4H), 7.14 (d, J = 2.3 Hz, 1H), 6.88 (d, J = 8.9 Hz, 1H), 6.78 (dd, J = 8.8, 2.4 Hz, 1H), 6.38 (s, 1H), 3.83 (dt, J = 11.5, 3.1 Hz, 2H), 3.36-3.19 (m, 3H), 2.83-2.81 (m, 1H), 1.72-1.55 (m, 3H). LCMS m/z 420.52 [M + H]⁺ |
| S5 | 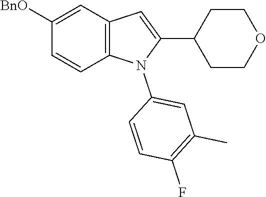 | 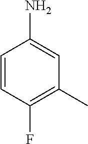 | ¹H NMR (400 MHz, Chloroform-d) δ 7.56-7.30 (m, 6H), 7.17 (d, J = 2.3 Hz, 1H), 7.07-6.83 (m, 5H), 6.42 (d, J = 0.8 Hz, 1H), 5.14 (s, 2H), 4.01 (ddd, J = 11.7, 4.3, 1.8 Hz, 2H), 3.41 (td, J = 11.8, 2.4 Hz, 2H), 2.88 (tt, J = 11.5, 4.0 Hz, 1H), 1.99-1.68 (m, 4H), 1.60 (s, 1H). LCMS m/z 402.0 [M + H]⁺. |
| S6 | 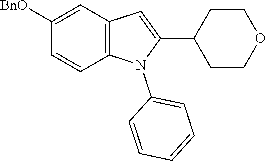 [SMILES: c1ccc(COc2ccc3c(c2)cc(C2CCOCC2)n3-c2ccccc2)cc1] | 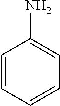 | ¹H NMR (400 MHz, Chloroform-d) δ 7.62-7.53 (m, 2H), 7.51-7.47 (m, 3H), 7.42- 7.31 (m, 4H), 7.17 (dd, J = 2.4, 0.6 Hz, 1H), 6.94 (dt, J = 8.9, 0.7 Hz, 1H), 6.85 (dd, J = 8.9, 2.4 Hz, 1H), 6.39 (t, J = 0.8 Hz, 1H), 5.13 (s, 2H), 4.00-3.84 (m, 2H), 3.34 (td, J = 11.8, 2.4 Hz, 2H), 2.91-2.72 (m, 2H), 1.90-1.69 (m, 4H). LCMS m/z 384.29 [M + H]⁺. |

### Preparation of S7

### 5-(methoxymethoxy)-1-(2-methylpyridin-4-yl)-2-(tetrahydro-2H-pyran-4-yl)-1H-indole (S7)

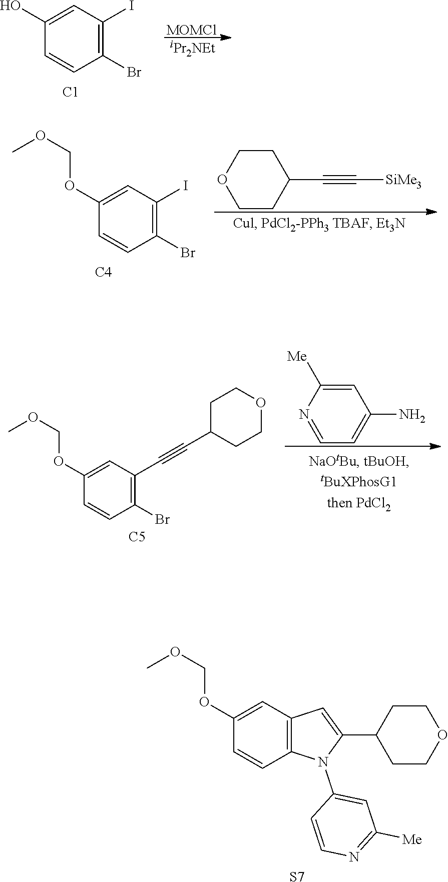

### Step 1. Synthesis of 1-bromo-2-iodo-4-(methoxymethoxy) benzene (C4)

To a cold (0′° C.) solution of 4-bromo-3-iodo-phenol (300.7 g, 1.006 mol) in CH₂Cl₂ (2.5 L) was added ⁱPr₂NEt (185.0 mL, 1.062 mol) followed by chloromethyl methyl ether (80 mL, 1.053 mol) at a rate to keep the temperature below 10° C. After the addition, the reaction was removed from the cooling bath and stirred at room temperature overnight. The resulting dark reddish-brown solution was poured into a separatory funnel and washed with 1N citric acid. The organic layer was separated and washed with 1N NaOH. The organic layer was isolated, dried (MgSO₄), and filtered over a short plug of silica gel. The plug was eluted with CH₂Cl₂ and the filtrate was evaporated in vacuo to afford 309.5 g of product. 1-bromo-2-iodo-4-(methoxymethoxy)benzene (90%). ¹H NMR (300 MHz, Chloroform-d) δ 7.55 (d, J=2.8 Hz, 1H), 7.48 (d, J=8.8 Hz, 1H), 6.90 (dd, J=8.8, 2.9 Hz, 1H), 5.12 (s, 2H), 3.46 (s, 3H).

### Step 2. 4-((2-bromo-5-(methoxymethoxy)phenyl)ethynyl)tetrahydro-2H-pyran (C5)

To a solution of 1-bromo-2-iodo-4-(methoxymethoxy)benzene C4 (2.0 g, 5.8 mmol) and trimethyl(2-tetrahydropyran-4-ylethynyl)silane (1.4 g, 7.6 mmol) in triethylamine (14 mL) was added water (0.21 mL, 11.68 mmol). To the mixture was added iodocopper (0.12 g, 0.65 mmol) and dichloropalladium; triphenylphosphane (0.21 g, 0.29 mmol). The mixture was purged with nitrogen for 2 minutes and tetrabutylammonium fluoride (7.6 mL of 1 M solution, 7.6 mmol) was added. The resulting black mixture was stirred at room temperature overnight. The solvents were removed in vacuo and the residue was diluted with CH₂Cl₂ and filtered through a pad of celite. The filtrate was concentrated in vacuo and the resulting crude material was purified by silica gel chromatography (80 g ISCO column) using a 0-50% EtOAc/heptanes gradient to afford 1.8 g of product. 4-[2-[2-bromo-5-(methoxymethoxy)phenyl]ethynyl]tetrahydropyran (95%). ¹H NMR (300 MHz, Chloroform-d) δ 7.46 (d, J=8.9 Hz, 1H), 7.15 (d, J=3.0 Hz, 1H), 6.86 (dd, J=8.8, 3.0 Hz, 1H), 5.16 (s, 2H), 4.01 (ddd, J=11.6, 6.5, 3.5 Hz, 2H), 3.62 (ddd, J=11.3, 7.6, 3.3 Hz, 2H), 3.48 (s, 3H), 2.96 (tt, J=8.0, 4.2 Hz, 1H), 1.97 (ddt, J=13.8, 7.1, 3.8 Hz, 2H), 1.89-1.71 (m, 2H).

### Step 3. 5-(methoxymethoxy)-1-(2-methylpyridin-4-yl)-2-(tetrahydro-2H-pyran-4-yl)-1H-indole (S7)

To a solution of 4-((2-bromo-5-(methoxymethoxy)phenyl)ethynyl)tetrahydro-2H-pyran C5 (5.02 g, 15.44 mmol) in tert-BuOH (50 mL) was added 2-methylpyridin-4-amine (1.70 g, 15.72 mmol) followed by NaOtBu (4.41 g, 45.89 mmol). tBuXPhos Pd G1 (0.59 g, 0.86 mmol) was added and the mixture was heated and stirred at reflux overnight to drive the reaction to completion. The crude reaction was poured into water. The mixture was extracted with CH₂Cl₂. The organic phase was dried (MgSO₄), filtered, and evaporated in vacuo to afford a dark red oil. The oil was dissolved in CH₂Cl₂ and filtered over a plug of silica gel. The plug was eluted with 25% EtOAc/CH₂Cl₂ and the filtrate was evaporated in vacuo to afford the crude product as a light red solid. The resulting solid was dissolved in CH₂Cl₂ and purified by silica gel chromatography (330 g ISCO silica gel cartridge) using 10% EtOAc/CH₂Cl₂ to elute impurities followed by 25% EtOAc/CH₂Cl₂ used to elute the product as a light yellow solid. The solid was triturated with pentane, filtered, and concentrated in vacuo to afford 6.0 g of product. 5-(methoxymethoxy)-1-(2-methyl-4-pyridyl)-2-tetrahydropyran-4-yl-indole (110%). ¹H NMR (400 MHz, Chloroform-d) δ 8.69 (d, J=5.3 Hz, 1H), 7.28 (d, J=2.2 Hz, 1H), 7.18 (d, J=1.9 Hz, 1H), 7.12 (dd, J=5.3, 1.6 Hz, 1H), 7.03 (d, J=8.9 Hz, 1H), 6.88 (dd, J=8.9, 2.4 Hz, 1H), 6.43 (s, 1H), 5.19 (s, 2H), 3.98 (dd, J=11.7, 2.5 Hz, 2H), 3.51 (s, 3H), 3.36 (td, J=11.8, 2.4 Hz, 2H), 2.90 (tt, J=11.4, 3.9 Hz, 1H), 2.67 (s, 3H), 1.88-1.65 (m, 4H). ESI-MS m/z calc. 352.18, found 353.33 (M+1)⁺.

### Preparation of S8-S11

### 5-(benzyloxy)-1-(4-fluoro-3-methylphenyl)-2-isopropyl-1H-indole (S8)

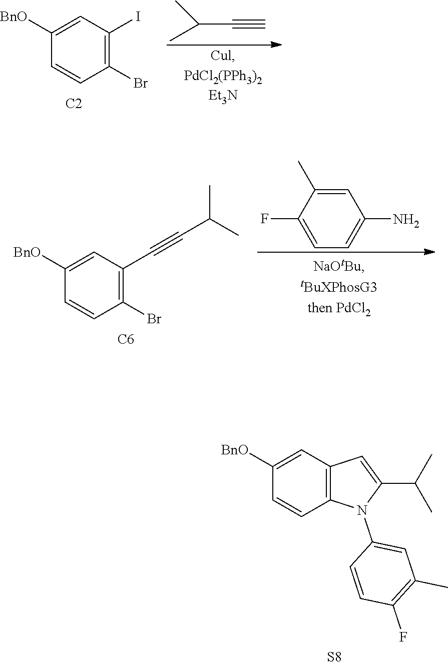

### Step 1. Synthesis of 4-(benzyloxy)-1-bromo-2-(3-methylbut-1-yn-1-yl)benzene (C6)

To a solution of 4-benzyloxy-1-bromo-2-iodo-benzene (172.0 g, 442.1 mmol) in triethylamine (1.5 L) in a 3 L round-bottomed flask was added 3-methylbut-1-yne (40.0 g, 563.7 mmol) followed by CuI (12.0 g, 63.0 mmol) and PdCl₂(PPh₃)₂ (17.4 g, 24.8 mmol). The solution was stirred overnight at room temperature. A solid precipitated during this time. The reaction was stripped of solvent and suspended in 20% CH₂Cl₂/heptanes; Loaded onto silica gel plug (˜1.5 Kg), and eluted with heptanes (2×1 L) and then eluted with 20% CH₂Cl₂/heptanes until no more pure product eluted. Pure fractions were combined to give a waxy tan colored solid that was dried to afford 140 g of product. 4-benzyloxy-1-bromo-2-(3-methylbut-1-ynyl)benzene (92%). ¹H NMR (300 MHz, Chloroform-d) δ 7.51-7.31 (m, 6H), 7.08 (d, J=3.0 Hz, 1H), 6.78 (dd, J=8.9, 3.1 Hz, 1H), 5.04 (s, 2H), 2.85 (hept, J=6.9 Hz, 1H), 1.33 (d, J=6.9 Hz, 6H). ESI-MS m/z calc. 328.04, found 338.56 (M+1)⁺.

### Step 2. Synthesis of 5-(benzyloxy)-1-(4-fluoro-3-methylphenyl)-2-isopropyl-1H-indole (S8)

To a solution of 4-benzyloxy-1-bromo-2-(3-methylbut-1-ynyl)benzene C6 (57.4 g, 165.6 mmol) in tert-BuOH (1 L) in a 1 L round bottom flask was added 4-fluoro-3-methyl-aniline (25.0 g, 199.8 mmol). The mixture was heated to 80° C. and NaOtBu (49.0 g, 494.6 mmol) was added. The mixture was purged with nitrogen for 10 minutes and then t-BuXPhos Pd G1 (5.3 g, 7.7 mmol) was added and the reaction heated to reflux overnight. Stripped off most of the solvent by first passing nitrogen to cool the reaction; then reducing the volume to 200 mL using rotoevaporation. The residue was dissolved in CH₂Cl₂ (500 mL) and filtered through a 500 g pad of silica gel. The silica pad was washed with CH₂Cl₂ (˜3×500 mL). The filtrate was concentrated in vacuo to afford 72 g of a dark brown solid. ¹H NMR showed the material to be a 2:1 mixture of uncyclized intermediate and closed indole S8. The residue was dissolved in DMSO (116 mL) to give ˜0.7 M solution that was heated to 150° C. for 30 minutes then cooled to room temperature. The reaction mixture was partitioned between aqueous saturated NaCl solution and 10% EtOAc/CH₂Cl₂. The aqueous phase was extracted multiple times with CH₂Cl₂ until no more UV material is seen. Organic extracts were combined, dried (Na₂SO₄), filtered and concentrated in vacuo. The resulting crude material was triturated between 1 L of 5% CH₂Cl₂/heptanes. Filtered solids washed with heptanes, then air dried by passing air over solid for 30 minutes. 36.2 g of a grey solid was obtained after drying. 5-benzyloxy-1-(4-fluoro-3-methyl-phenyl)-2-isopropyl-indole (62%). ¹H NMR (300 MHz, DMSO-d6) δ 7.37 (ddt, J=21.3, 11.8, 7.2 Hz, 9H), 7.12 (s, 1H), 6.77 (q, J=8.8 Hz, 2H), 6.32 (s, 1H), 5.10 (s, 2H), 3.01-2.78 (m, 1H), 2.31 (s, 3H), 1.14 (d, J=6.6 Hz, 6H). ESI-MS m/z calc. 373.18, found 374.41 (M+1)⁺.

Compounds S9-S11 (Table 2) were made by a similar method to S8, substituting the appropriate aniline into the amination step.

TABLE 2
Structure and physicochemical data for intermediates S9-S11

| Intermediate | Structure | Aniline | ¹H NMR; LCMS m/z [M + H]⁺ |
|---|---|---|---|
| S9 | 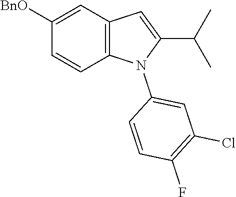 [SMILES: CC(C)c1cc2cc(OCc3ccccc3)ccc2n1-c1ccc(F)c(Cl)c1] | 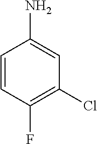 | ¹H NMR (400 MHz, DMSO-d6) δ 7.78 (dd, J = 6.6, 2.6 Hz, 1H), 7.64 (t, J = 8.9 Hz, 1H), 7.52- 7.42 (m, 3H), 7.42-7.34 (m, 2H), 7.34-7.27 (m, 1H), 7.13 (dd, J = 2.4, 0.6 Hz, 1H), 6.85 (dt, J = 8.8, 0.6 Hz, 1H), 6.77 (dd, J = 8.9, 2.4 Hz, 1H), 6.35 (t, J = 0.8 Hz, 1H), 5.11 (s, 2H), 2.99-2.80 (m, 1H), 1.14 (d, J= 6.8 Hz, 6H). LCMS m/z 393.21 [M + H]⁺. |
| S10 | 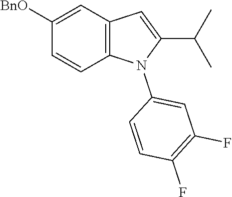 [SMILES: CC(C)c1cc2cc(OCc3ccccc3)ccc2n1-c1ccc(F)c(F)c1] | 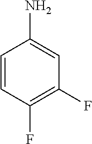 | ¹H NMR (400 MHz, Chloroform-d) δ 7.57-7.33 (m, 7H), 7.16 (d, J = 2.3 Hz, 1H), 6.99-6.80 (m, 2H), 6.38 (s, 1H), 5.13 (s, 2H), 3.02-2.84 (m, 1H), 1.58 (d, J = 0.9 Hz, 3H), 1.22 (d, J = 6.8 Hz, 6H). LCMS m/z 378.0 [M + H]⁺. |
| S11 | 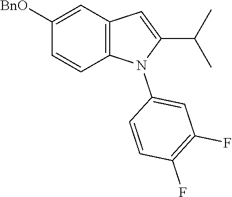 [SMILES: CC(C)c1cc2cc(OCc3ccccc3)ccc2n1-c1ccc(F)c(F)c1] | 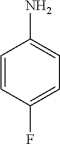 | ¹H NMR (300 MHz, Chloroform-d) δ 7.54-7.12 (m, 10H), 6.95-6.78 (m, 2H), 5.13 (s, 2H), 2.91 (p, J = 6.8 Hz, 1H), 1.21 (d, J = 6.8 Hz, 6H). LCMS m/z 360.2 [M + H]⁺. |

### Preparation of S12

### 2-isopropyl-5-(methoxymethoxy)-1-(2-methylpyridin-4-yl)-1H-indole (S12)

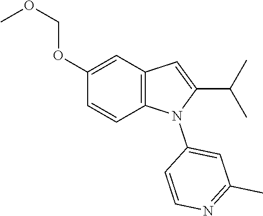

S12 is made by a similar method to S8 using OMOM as replacement for OBn and 2-methylpyridin-4-amine as a replacement for 4-fluoro-3-methyl-aniline. Core made by Sonagashira, Buchwald, cyclization. 1-(4-fluoro-3-methylphenyl)-2-isopropyl-5-(methoxymethoxy)-1H-indole. ¹H NMR (300 MHz, Chloroform-d) δ 8.67 (dd, J=5.3, 0.7 Hz, 1H), 7.31-7.24 (m, 1H), 7.22-7.15 (m, 1H), 7.13 (ddd, J=5.3, 2.0, 0.6 Hz, 1H), 7.03 (dt, J=8.8, 0.7 Hz, 1H), 6.85 (dd, J=8.8, 2.4 Hz, 1H), 6.41 (t, J=0.8 Hz, 1H), 5.19 (s, 2H), 3.51 (s, 3H), 3.03 (pd, J=6.8, 0.8 Hz, 1H), 2.66 (s, 3H), 1.20 (d, J=6.8 Hz, 6H). ESI-MS m/z calc. 310.17, found 311.35 (M+1)⁺.

### Preparation of S13-S15

### 5-(benzyloxy)-1-(4-fluoro-3-methylphenyl)-2-(1-methoxy-2-methylpropan-2-yl)-1H-indole (S13)

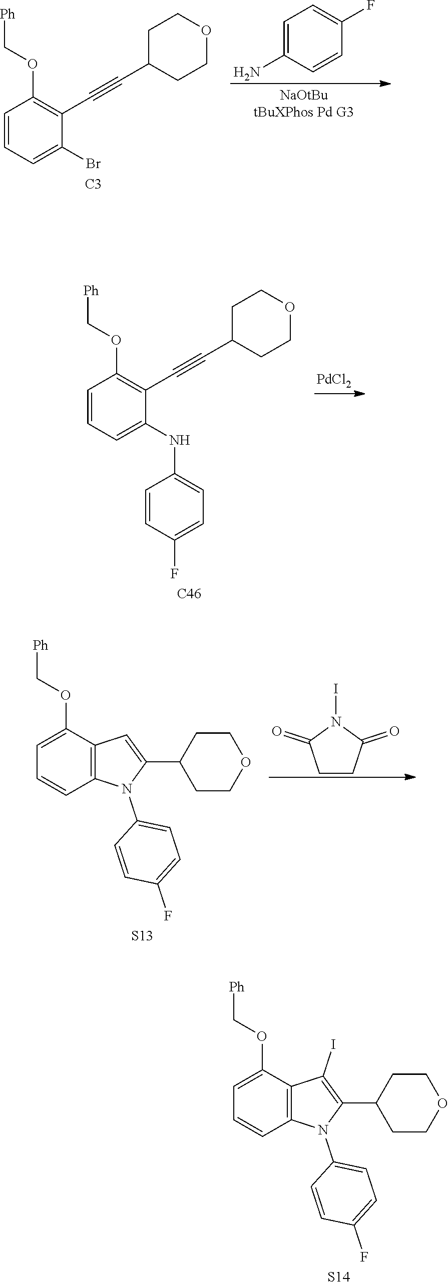

### Step 1. Synthesis of 4-(5-(benzyloxy)-2-bromophenyl)-2,2-dimethylbut-3-yn-1-ol (C7)

To a solution of 4-benzyloxy-1-bromo-2-iodo-benzene C2 (13.3 g, 34.2 mmol) and 2,2-dimethylbut-3-yn-1-ol (4.0 g, 40.8 mmol) in dioxane (75 mL) was added iPr₂NEt (15.0 mL, 86.1 mmol). The reaction mixture was purged with nitrogen for 5-10 minutes. PdCl₂(PPh₃)₂ (1.2 g, 1.7 mmol) was added followed by CuI (0.7 g, 3.7 mmol). The reaction mixture was stirred at room temperature under nitrogen and foil overnight. The reaction was filtered with the aid of EtOAc and then concentrated in vacuo. Purification by silica gel chromatography (330 g ISCO column) using 0-100% EtOAc/heptanes gradient to afford 7.2 g of product. 4-(5-benzyloxy-2-bromo-phenyl)-2,2-dimethyl-but-3-yn-1-ol (81%). ¹H NMR (400 MHz, Chloroform-d) δ 7.45 (d, J=8.9 Hz, 1H), 7.44-7.34 (m, 5H), 7.09 (d, J=3.0 Hz, 1H), 6.82 (dd, J=8.9, 3.0 Hz, 1H), 5.05 (s, 2H), 3.55 (d, J=7.2 Hz, 2H), 2.10 (d, J=7.1 Hz, 1H), 1.35 (s, 6H). ESI-MS m/z calc. 358.06, found 359.17 (M+1)⁺.

### Step 2. Synthesis of 4-(benzyloxy)-1-bromo-2-(4-methoxy-3,3-dimethylbut-1-yn-1-yl)benzene (C8)

To a solution/suspension of 4-(5-benzyloxy-2-bromo-phenyl)-2,2-dimethyl-but-3-yn-1-ol C₇ (7.2 g, 19.9 mmol) and 1-(bromomethyl)-4-methoxy-benzene (3.2 mL, 21.9 mmol) in 2-MeTHF (40 mL) was added at room temperature NaH (0.8 g of 60% w/w, 20.9 mmol). The reaction mixture increased in temperature to ˜35° C. Water and EtOAc were added and the layers were separated. The aqueous layer was re-extracted with EtOAc and the combined organic phases were concentrated in vacuo. The resulting residue was purified by silica gel chromatography (220 g ISCO column) using a 0-100% EtOAc/heptanes gradient to afford 1.71 g of product. The methylated product was obtained. 4-benzyloxy-1-bromo-2-(4-methoxy-3,3-dimethyl-but-1-ynyl)benzene (23%). ¹H NMR (400 MHz, Chloroform-d) δ 7.45-7.33 (m, 7H), 7.09 (d, J=3.1 Hz, 1H), 6.78 (dd, J=8.9, 3.0 Hz, 1H), 5.04 (s, 2H), 3.47 (s, 3H), 3.40 (s, 2H), 1.36 (s, 6H). ESI-MS m/z calc. 372.07, found 375.24 (M+1)⁺.

### Step 3. Synthesis of 4-(benzyloxy)-N-(4-fluoro-3-methylphenyl)-2-(4-methoxy-3,3-dimethylbut-1-yn-1-yl)aniline (C8)

A solution of 4-benzyloxy-1-bromo-2-(4-methoxy-3,3-dimethyl-but-1-ynyl)benzene C₇ (1.71 g, 4.58 mmol) and 4-fluoro-3-methyl-aniline (0.64 g, 5.08 mmol) in dioxane (5 mL) and tert-BuOH (5 mL) was purged with nitrogen for 5-10 minutes. During the purge was added sequentially, tBuXphos Pd G1 (0.20 g, 0.29 mmol) followed by sodium tert-butoxide (1.00 g, 10.41 mmol). The reaction mixture was stirred under nitrogen for 4 hours at room temperature. The reaction mixture was filtered through Celite with the aid of EtOAc and then concentrated in vacuo. Purification by silica gel chromatography (80 g GOLD column) 0-100% EtOAc/heptanes gradient afforded 1.91 g of product. of 4-(benzyloxy)-N-(4-fluoro-3-methylphenyl)-2-(4-methoxy-3,3-dimethylbut-1-yn-1-yl)aniline (100%). ¹H NMR (400 MHz, Chloroform-d) δ 7.46-7.38 (m, 4H), 7.37-7.32 (m, 1H), 7.05 (d, J=8.9 Hz, 1H), 7.01 (d, J=2.9 Hz, 1H), 6.97-6.90 (m, 3H), 6.84 (dd, J=9.0, 3.0 Hz, 1H), 5.02 (s, 2H), 3.43 (s, 3H), 3.34 (s, 2H), 2.29-2.24 (m, 3H), 1.34 (s, 6H). ESI-MS m/z calc. 417.21, found 418.41 (M+1)⁺.

### Step 4. 5-(benzyloxy)-1-(4-fluoro-3-methylphenyl)-2-(1-methoxy-2-methylpropan-2-yl)-1H-indole (S13)

To a solution of N-[4-benzyloxy-2-(4-methoxy-3,3-dimethyl-but-1-ynyl)phenyl]-4-fluoro-3-methyl-aniline C8 (1.23 g, 2.946 mmol) in 2-MeTHF (20 mL) was added KOt-Bu (3.25 mL of 1 M solution, 3.25 mmol). The reaction mixture was heated at 50° C. until reaction ran to completion.

Water and CH₂Cl₂ were added and the layers were separated with the aid of a phase separator. The aqueous layer was re-extracted with CH₂Cl₂ and the layers were separated through a phase separator again and the combined organics concentrated. MTBE was added and an off-white solid was filtered off to afford 800 mg of product. 5-benzyloxy-1-(4-fluoro-3-methyl-phenyl)-2-(2-methoxy-1,1-dimethyl-ethyl)indole (65%). ¹H NMR (400 MHz, Chloroform-d) δ 7.48 (ddt, J=7.5, 1.4, 0.7 Hz, 2H), 7.42-7.37 (m, 2H), 7.35-7.30 (m, 1H), 7.21 (tq, J=7.5, 2.1 Hz, 2H), 7.16-7.13 (m, 1H), 7.12 (d, J=2.3 Hz, 1H), 6.79 (dd, J=8.8, 2.4 Hz, 1H), 6.57 (dt, J=8.9, 0.6 Hz, 1H), 6.43 (d, J=0.8 Hz, 1H), 5.11 (s, 2H), 3.25 (s, 3H), 3.19 (s, 2H), 2.35 (d, J=2.0 Hz, 3H), 1.30 (s, 3H), 1.28 (s, 3H). ESI-MS m/z calc. 417.21, found 418.41 (M+1)⁺.

Compounds S14-S15 (Table 3) were made by a similar method to S13, substituting the appropriate alkyne into the Sonagashira coupling step.

TABLE 3
Structure and physicochemical data for intermediates S14-S15

| Intermediate | Structure | Alkyne | ¹H NMR; LCMS m/z [M + H]⁺ |
|---|---|---|---|
| S14 | 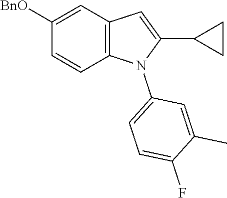 [SMILES: Cc1cc(-n2c(C3CC3)cc3cc(OCc4ccccc4)ccc32)ccc1F] | 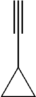 | ¹H NMR (400 MHz, Chloroform-d) δ 7.46 (dd, J = 7.9, 1.0 Hz, 2H), 7.41-7.33 (m, 2H), 7.33-7.17 (m, 3H), 7.13 (t, J = 8.8 Hz, 1H), 7.08 (d, J = 2.3 Hz, 1H), 6.96 (d, J = 8.8 Hz, 1H), 6.81 (dd, J = 8.8, 2.4 Hz, 1H), 6.08 (s, 1H), 5.09 (s, 2H), 2.34 (d, J = 1.9 Hz, 3H), 1.71-1.57 (m, 1H), 0.97-0.68 (m, 4H). LCMS m/z 372.0 [M + H]⁺. |
| S15 | 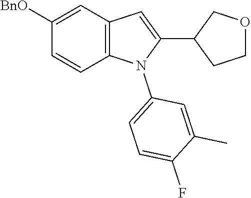 [SMILES: Cc1cc(-n2c(C3CCOC3)cc3cc(OCc4ccccc4)ccc32)ccc1F] | 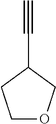 | ¹H NMR (400 MHz, Chloroform-d) δ 7.53-7.48 (m, 2H), 7.46-7.38 (m, 2H), 7.39-7.32 (m, 1H), 7.21- 7.09 (m, 4H), 6.95-6.82 (m, 2H), 6.42 (d, J = 0.8 Hz, 1H), 5.14 (s, 2H), 4.07-3.92 (m, 2H), 3.86 (dt, J = 8.4, 7.3 Hz, 1H), 3.80-3.69 (m, 1H), 3.36 (p, J = 7.8 Hz, 1H), 2.38 (d, J = 2.0 Hz, 3H), 2.26-2.16 (m, 1H), 2.15-2.04 (m, 1H). LCMS m/z 401.5 [M + H]⁺. |

### Preparation of S16

### Synthesis of 5-(benzyloxy)-1-(4-fluoro-3-methylphenyl)-1H-indole (S16)

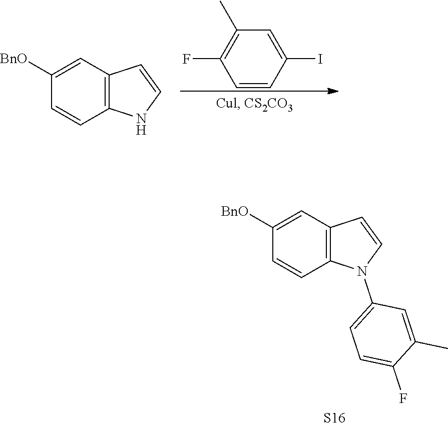

To a solution of 5-benzyloxy-1H-indole (10.0 g, 44.8 mmol) and 1-fluoro-4-iodo-2-methyl-benzene (12.0 g, 50.8 mmol) in DMF (50 mL) was added CuI (0.5 g, 2.6 mmol) and Cs₂CO₃ (25.0 g, 76.7 mmol). The mixture was purged with nitrogen for 5 minutes in a pressure bottle (Qian cap) which was then sealed and heated at 130° C. for 24 h. The solution was diluted with EtOAc (200 mL) and the solid was filtered. The filtrate was washed with water (200 mL) and the organic layer was separated. and the aqueous layer was extracted with EtOAc (2×100 mL). The combined organic layers were dried over Na₂SO₄, filtered and concentrated under reduced pressure. The crude residue was purified by silica gel chromatography (80 g ISCO column) eluting with 0-15% EtOAc/heptanes to afford 7.8 g of product as a white solid. 5-benzyloxy-1-(4-fluoro-3-methyl-phenyl)indole (51%). ESI-MS m/z calc. 331.14, found 326.11 (M+1)⁺.

### Preparation of S17

### 5-(benzyloxy)-4-fluoro-1-(4-fluoro-3-methylphenyl)-2-isopropyl-1H-indole (S17)

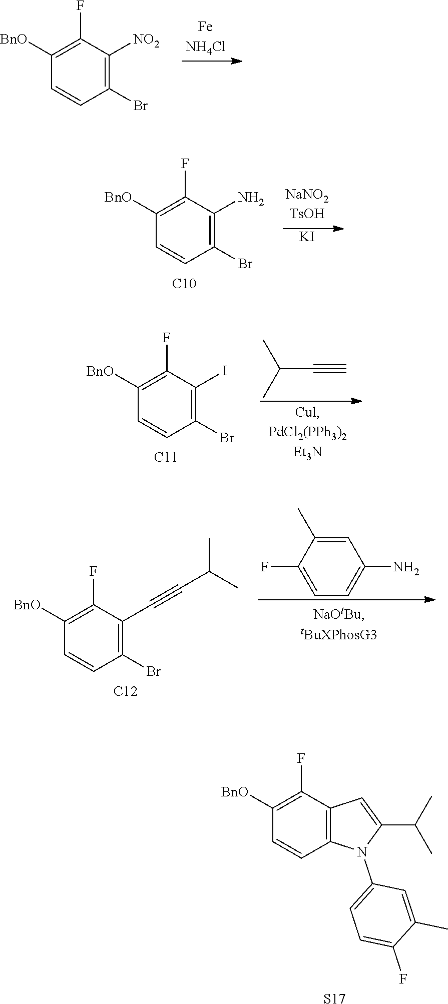

### Step 1. Synthesis of 3-(benzyloxy)-6-bromo-2-fluoroaniline (C10)

To a solution of 1-benzyloxy-4-bromo-2-fluoro-3-nitro-benzene (4.96 g, 15.21 mmol), Fe (4.25 g, 76.10 mmol) in methanol (150 mL) was added NH₄Cl (4.09 g, 76.46 mmol). The reaction mixture was heated to 70° C. overnight. After cooling to room temperature, the mixture was filtered through a pad of celite and the resulting solid was washed with methanol. The filtrate was concentrated in vacuo and then diluted into H₂O and extracted with EtOAc. The organic phase was washed with brine, dried (MgSO₄), filtered, and concentrated in vacuo. The resulting crude material was purified by silica gel chromatography (330 g ISCO column) using 0-15% EtOAc/heptanes gradient to afford 4.02 g of product that formed into a white solid upon drying. 3-benzyloxy-6-bromo-2-fluoro-aniline (88%). ¹H NMR (400 MHz, Chloroform-d) δ 7.48-7.29 (m, 5H), 7.06 (dd, J=8.9, 1.3 Hz, 1H), 6.38-6.28 (m, 1H), 5.11 (s, 2H), 4.12 (s, 2H). ESI-MS m/z calc. 295.0, found 296.5 (M+1)⁺.

### Step 2. Synthesis of 1-(benzyloxy)-4-bromo-2-fluoro-3-iodobenzene (C11)

To a cold (−5° C.) suspension of 3-benzyloxy-6-bromo-2-fluoro-aniline C10 (3.28 g, 10.92 mmol) and TsOH—H₂O (6.24 g, 32.80 mmol) in acetonitrile (100 mL) was added dropwise a solution of NaNO₂ (1.51 g, 21.89 mmol) and KI (4.53 g, 27.29 mmol) in water (7.0 mL) at a rate of 0.20 mL/min with a syringe pump. Internal temp was <−5° C. for entire addition. The reaction mixture turned yellow, then black, then dark orange over time. The reaction mixture was allowed to slowly warm to room temperature overnight. The solvent was removed under reduced pressure and the resulting crude was diluted into water and extracted with EtOAc. The organic phase was washed with brine, dried (MgSO₄), filtered, and concentrated in vacuo. The resulting residue was purified by silica gel chromatography (80 g ISCO column) using a 0-10% EtOAc/heptanes gradient to yield material that was still impure. A second purification by silica gel chromatography (80 g ISCO column) using 0-40% CHCl3/heptanes afforded product as a clear colorless oil. 1-(benzyloxy)-4-bromo-2-fluoro-3-iodobenzene. ¹H NMR (300 MHz, Chloroform-d) δ 7.45-7.29 (m, 6H), 6.88 (dd, J=8.8, 8.3 Hz, 1H), 5.13 (s, 2H).

### Step 3. Synthesis of 1-(benzyloxy)-4-bromo-2-fluoro-3-(3-methylbut-1-yn-1-yl)benzene (C12)

To a solution of 1-benzyloxy-4-bromo-2-fluoro-3-iodo-benzene C11 (1.08 g, 2.63 mmol) in triethylamine (7.0 mL) purged with nitrogen for 5 minutes was added Pd(PPh₃)₂Cl₂ (0.09 g, 0.13 mmol), CuI (0.03 g, 0.13 mmol) and 3-methylbut-1-yne (0.33 mL, 3.18 mmol). The reaction mixture was heated at 40° C. overnight. LCMS shows reaction did not go to completion. Additional 3-methylbut-1-yne (0.33 mL, 3.18 mmol), Pd(PPh₃)₂Cl₂ (0.09 g, 0.13 mmol) and CuI (0.03 g, 0.13 mmol) were added to the reaction mixture. The reaction mixture was heated at 40° C. overnight again. Removed solvent in vacuo. Added H₂O and extracted with EtOAc. Combined organic phases were washed with 1M HCl and then brine, dried (MgSO₄), filtered, and concentrated in vacuo. The resulting residue was purified by silica gel chromatography (120 g ISCO column) using CHCl₃/heptanes gradient to afford 548 mg of desired product. 1-benzyloxy-4-bromo-2-fluoro-3-(3-methylbut-1-ynyl)benzene (60%). ¹H NMR (400 MHz, Chloroform-d) δ 7.43-7.29 (m, 5H), 7.2 (dd, J=8.9, 1.9 Hz, 1H), 6.77 (dd, J=8.9, 8.3 Hz, 1H), 5.12 (s, 2H), 2.87 (heptd, J=6.9, 0.9 Hz, 1H), 1.31 (d, J=6.9 Hz, 6H). ESI-MS m/z calc. 346.0, found 346.9 (M+1)⁺.

### Step 4. Synthesis of 5-(benzyloxy)-4-fluoro-1-(4-fluoro-3-methylphenyl)-2-isopropyl-1H-indole (S17)

To a solution of 1-benzyloxy-4-bromo-2-fluoro-3-(3-methylbut-1-ynyl)benzene Cl₂ (0.55 g, 1.58 mmol) in dioxane (7 mL) was added 4-fluoro-3-methyl-aniline (0.23 g, 1.84 mmol). The mixture was degassed with nitrogen for 10 minutes. tBuXPhos Pd G3 (0.06 g, 0.08 mmol) and NaOtBu (0.46 g, 4.74 mmol) were added to the mixture which was purged again with nitrogen. The reaction mixture was sealed and heated to 80° C. After 10 minutes, the reaction was cooled to room temperature. The mixture was filtered through a pad of fluorosil and washed with CH₂Cl₂/EtOAc. The filtrate was concentrated in vacuo. The resulting residue was diluted into water and extracted with EtOAc. Combined organic phases were washed with brine, dried (MgSO₄), filtered and concentrated in vacuo. The resulting residue was purified by silica gel chromatography (40 g ISCO column) using 0-20% EtOAc/heptanes gradient to afford 515 mg of desired product. 5-benzyloxy-4-fluoro-1-(4-fluoro-3-methyl-phenyl)-2-isopropyl-indole (82%). ESI-MS m/z calc. 391.17, found 391.36 (M+1)⁺.

Compounds S18-S20 were made by a similar method to S17, using the appropriate iodoaniline (Table 4) by Sonagashira coupling with isopropyl alkyne, followed by N-arylation with 4-bromo 2-methyl bromo benzene.

TABLE 4
Structure and physicochemical data for intermediates S18-S20

| Intermediate | Structure | Iodide | ¹H NMR; LCMS m/z [M + H]⁺ |
|---|---|---|---|
| S18 | 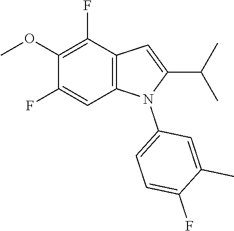 | 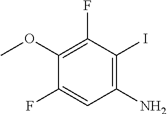 | ¹H NMR (400 MHz, Chloroform-d) δ 8.09-7.99 (m, 1H), 7.35-7.18 (m, 2H), 7.13-6.91 (m, 4H), 6.38 (ddd, J = 10.6, 1.5, 0.8 Hz, 1H), 6.32 (t, J = 0.8 Hz, 1H), 2.86-2.62 (m, 1H), 2.26 (dd, J = 12.7, 2.1 Hz, 4H), 1.08 (dd, J = 6.9, 3.7 Hz, 6H). LCMS m/z 334.5 [M + H]⁺. |
| S19 | 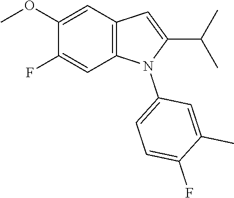 | 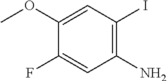 | ¹H NMR (400 MHz, Chloroform-d) δ 7.22-7.04 (m, 4H), 6.73 (dd, J = 11.6, 0.8 Hz, 1H), 6.34 (t, J = 0.8 Hz, 1H), 3.94 (s, 3H), 2.93- 2.89 (m, 1H), 2.37 (d, J = 2.0 Hz, 3H), 1.20 (d, J = 6.8 Hz, 6H). LCMS m/z 316.3 [M + H]⁺. |
| S20 | 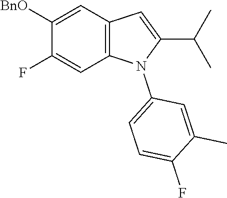 [SMILES: Cc1cc(-n2c(C(C)C)cc3cc(OCc4ccccc4)c(F)cc32)ccc1F] | 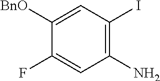 [SMILES: Nc1cc(F)c(OCc2ccccc2)cc1I] | ¹H NMR (400 MHz, Chloroform-d) δ 7.48 (d, J = 7.4 Hz, 2H), 7.38 (t, J =7.5 Hz, 2H), 7.34-7.27 (m, 1H), 7.22-7.07 (m, 3H), 6.82 (t, J = 8.2 Hz, 1H), 6.60 (d, J = 8.7 Hz, 1H), 6.49 (s, 1H), 5.15 (s, 2H), 2.89 (hept, J = 6.9 Hz, 1H), 2.36 (s, 3H), 1.22 (d, J = 5.4 Hz, 6H). |

### Preparation of S21

### Synthesis of 5-(benzyloxy)-6-fluoro-1-(4-fluoro-3-methylphenyl)-2-isopropyl-1H-indole (S21)

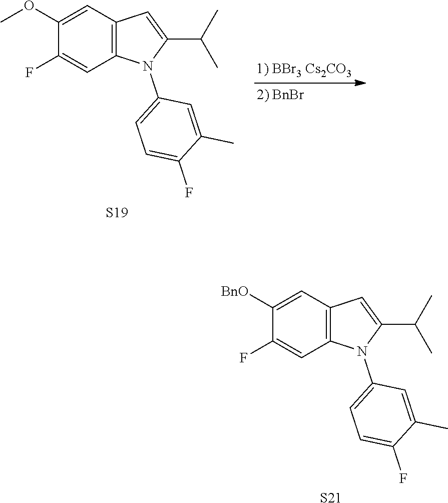

To a cold (0° C.) solution of 6-fluoro-1-(4-fluoro-3-methyl-phenyl)-2-isopropyl-5-methoxy-indole S19 (0.8 g, 2.5 mmol) in CH₂Cl₂ (25 mL) was added BBr₃ (5.0 mL of 1 M solution, 5.0 mmol). The reaction mixture was warmed to room temperature and stirred for 120 min. The mixture was washed with aqueous saturated NaHCO₃ solution. The organic phase was dried over Na₂SO₄, filtered and concentrated in vacuo to afford 6-fluoro-1-(4-fluoro-3-methyl-phenyl)-2-isopropyl-indol-5-ol. The crude product was dissolved in acetone (25 mL) and benzyl bromide (0.35 mL, 2.94 mmol) and CS₂CO₃ (1.6 g, 4.911 mmol) were added and the resulting solution was stirred at room temperature for 24 h. The mixture was diluted into water (25 mL) and extracted with EtOAc (3×25 mL). The combined organic phases were dried over Na₂SO₄ and concentrated in vacuo. The resulting residue was purified by silica gel chromatography using 0-50% EtOAc/heptanes gradient to afford 781 mg of product as a white solid. 5-benzyloxy-6-fluoro-1-(4-fluoro-3-methyl-phenyl)-2-isopropyl-indole (81%). ¹H NMR (400 MHz, Chloroform-d) δ 7.51 (d, J=7.5 Hz, 2H), 7.41 (t, J=7.4 Hz, 2H), 7.34 (t, J=7.3 Hz, 1H), 7.20-7.05 (m, 4H), 6.75 (d, J=11.5 Hz, 1H), 6.31 (s, 1H), 5.17 (s, 2H), 2.99-2.85 (m, 1H), 2.46-2.33 (m, 3H), 1.20 (d, J=6.8 Hz, 7H). ESI-MS m/z calc. 391.17, found 390.69 (M+1)⁺.

### Preparation of S22

### Synthesis of 4-(5-(benzyloxy)-1-(4-fluoro-3-methylphenyl)-1H-indol-2-yl)tetrahydro-2H-thiopyran 1,1-dioxide (S22)

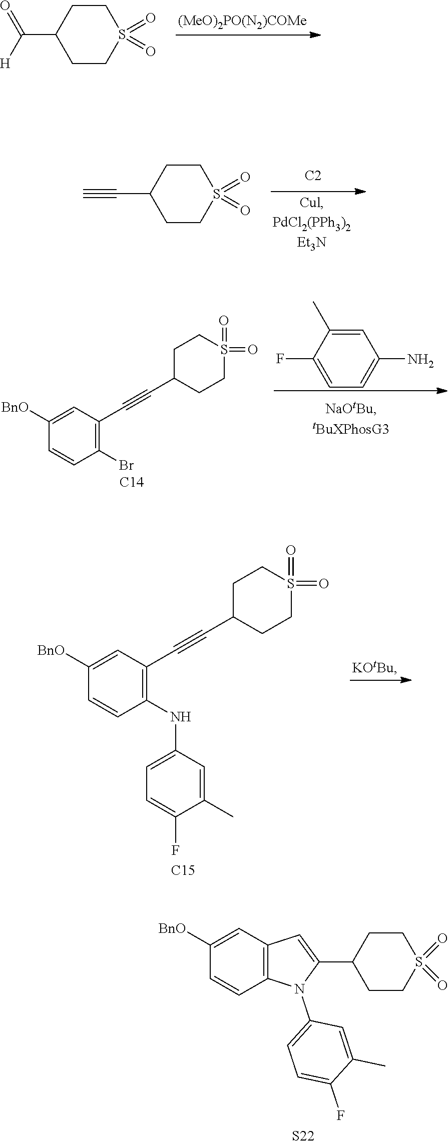

### Step 1. Synthesis of 4-ethynyltetrahydro-2H-thiopyran 1,1-dioxide (C13)

To a solution of 1,1-dioxothiane-4-carbaldehyde (2.93 g, 18.06 mmol), 1-diazo-1-dimethoxyphosphoryl-propan-2-one (5.20 g, 27.07 mmol) in methanol (20 mL) was added K₂CO₃ (5.00 g, 36.18 mmol). The reaction mixture was stirred at room temperature overnight. The reaction was concentrated in vacuo and the resulting residue was diluted with EtOAc and washed with water. The organic phase was dried over Na₂SO₄ and concentrated in vacuo. The resulting residue was purified by silica gel chromatography (4 g ISCO column) using 10-40% EtOAc/heptanes gradient to afford 2.28 g of desired product. 4-ethynylthiane 1,1-dioxide (80%). ¹H NMR (400 MHz, Methanol-d4) δ 4.92-4.76 (m, 1H), 3.14-3.04 (m, 4H), 2.44-2.33 (m, 2H), 2.10 (dtd, J=14.2, 10.1, 3.7 Hz, 2H).

### Step 2. Synthesis of 4-((5-(benzyloxy)-2-bromophenyl)ethynyl)tetrahydro-2H-thiopyran 1,1-dioxide (C14)

To a solution of 4-benzyloxy-1-bromo-2-iodo-benzene (3.50 g, 8.99 mmol) and 4-ethynyltetrahydro-2H-thiopyran 1,1-dioxide C₁₃ (1.98 g, 12.51 mmol) in trimethylamine (15 ml) and dioxane (15 ml) was added Pd(PPh₃)₂Cl₂ (0.61 g, 0.87 mmol) and CuI (0.31 g, 1.62 mmol). The reaction mixture was heated at 60° C. overnight. The reaction was cooled to room temperature and then filtered through a plug of celite. The filtrate was diluted with EtOAc and washed with water. The organic phase was dried over Na₂SO₄ and concentrated in vacuo. The resulting residue was purified by silica gel chromatography (40 g ISCO column) using 10-90% EtOAc/heptanes gradient to afford 2.1 g of product. 4-[2-(5-benzyloxy-2-bromo-phenyl)ethynyl]thiane 1,1-dioxide (51%) ESI-MS m/z calc. 418.02, found 419.35 (M+1)⁺.

### Step 3. Synthesis of 4-((5-(benzyloxy)-2-bromophenyl)ethynyl)tetrahydro-2H-thiopyran 1,1-dioxide (C15)

To a solution of 4-((5-(benzyloxy)-2-bromophenyl)ethynyl)tetrahydro-2H-thiopyran 1,1-dioxide C14 (2.09 g, 4.98 mmol) and 4-fluoro-3-methyl-aniline (0.65 g, 5.19 mmol) in t-BuOH (8 mL) and dioxane (8 mL) was added tBuXPhos Pd G3 (0.20 g, 0.25 mmol) and NaOtBu (1.25 g, 13.01 mmol). The reaction mixture was stirred at room temperature for overnight. The mixture was concentrated in vacuo and resulting residue was diluted with EtOAc and washed with water. The organic phase was dried over Na₂SO₄ and concentrated in vacuo. The resulting residue was purified by silica gel chromatography (40 g ISCO column) using a 10-40% EtOAc/heptanes gradient to 2.12 g of product. N-[4-benzyloxy-2-[2-(1,1-dioxothian-4-yl)ethynyl]phenyl]-4-fluoro-3-methyl-aniline (61%). ESI-MS m/z calc. 463.16, found 464.23 (M+1)⁺.

### Synthesis of 4-(5-(benzyloxy)-1-(4-fluoro-3-methylphenyl)-1H-indol-2-yl)tetrahydro-2H-thiopyran 1,1-dioxide (S22)

To a solution of N-[4-benzyloxy-2-[2-(1,1-dioxothian-4-yl)ethynyl]phenyl]-4-fluoro-3-methyl-aniline C15 (1.12 g, 2.42 mmol) in THE (20 mL) was added KOtBu (0.27 g, 2.40 mmol). The reaction mixture was stirred at room temperature overnight. The mixture was concentrated in vacuo, diluted with EtOAc and washed with water. The organic phase was dried over Na₂SO₄ and concentrated in vacuo. The resulting residue was purified by silica gel chromatography (40 g ISCO column) using a 10-40% EtOAc/heptanes gradient to afford 820 mg of product. 4-[5-benzyloxy-1-(4-fluoro-3-methyl-phenyl)indol-2-yl]thiane 1,1-dioxide (43%). ESI-MS m/z calc. 463.16, found 464.23 (M+1)⁺.

### Preparation of S23

### Synthesis of 2-isopropyl-5-methoxy-1-(2-methylpyrimidin-4-yl)-1H-indole (S23)

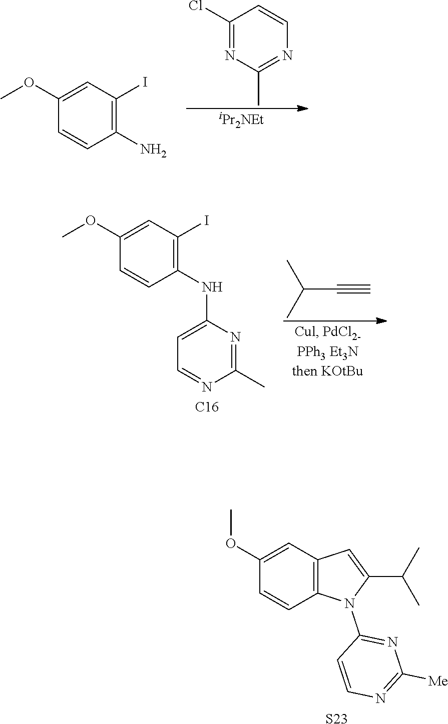

### Step 1. Synthesis of N-(2-iodo-4-methoxyphenyl)-2-methylpyrimidin-4-amine (C16)

A mixture of 2-iodo-4-methoxy-aniline (2.52 g, 10.12 mmol), 4-chloro-2-methyl-pyrimidine (1.80 g, 14.00 mmol) and iPr₂NEt (4.0 mL, 22.9 mmol) in DMSO (10 mL) was irradiated in microwave for 20 minutes at 180° C. The reaction mixture was cooled to room temperature, diluted with EtOAc, washed with H₂O, dried over Na₂SO₄, filtered and concentrated in vacuo. The resulting residue was purified by silica gel chromatography (40 g ISCO column) using 0-60% EtOAc/CH₂Cl₂ gradient to afford 1.0 g of product. N-(2-iodo-4-methoxy-phenyl)-2-methyl-pyrimidin-4-amine (29%). ESI-MS m/z calc. 341.0, found 342.0 (M+1)⁺.

### Step 2. Synthesis of N-(2-iodo-4-methoxyphenyl)-2-methylpyrimidin-4-amine (S23)

To a solution of N-(2-iodo-4-methoxy-phenyl)-2-methyl-pyrimidin-4-amine C16 (1.00 g, 2.93 mmol) and 3-methylbut-1-yne (0.40 g, 5.87 mmol) in trimethylamine (10 mL) was added Pd(PPh₃)₂Cl₂ (0.20 g, 0.28 mmol) and CuI (0.15 g, 0.79 mmol). The reaction mixture was heated at 50° C. for 1 hour. The mixture was concentrated in vacuo and diluted with EtOAc, filtered through a pad of celite and the filtrate concentrated in vacuo. The resulting residue was purified by silica gel chromatography (40 g ISCO column) using 0-100% EtOAc/heptanes gradient to afford 420 mg of product. N-[4-methoxy-2-(3-methylbut-1-ynyl)phenyl]-2-methyl-pyrimidin-4-amine (51%). ESI-MS m/z calc. 281.2, found 282.0 (M+1)⁺.

To a solution of N-[4-methoxy-2-(3-methylbut-1-ynyl)phenyl]-2-methyl-pyrimidin-4-amine (0.42 g) in THE (20 mL) was added KOtBu (0.40 g, 3.57 mmol. The reaction mixture was heated to reflux and maintained at that temperature overnight. The mixture was cooled, concentrated in vacuo and diluted with water. The aqueous phase was extracted with CH₂Cl₂ and the organic phase was dried (MgSO₄), filtered, and concentrated in vacuo. The resulting residue was purified by silica gel chromatography (12 g ISCO column) using 0-30% EtOAc/CH₂Cl₂ gradient to afford 320 mg of product. 2-isopropyl-5-methoxy-1-(2-methylpyrimidin-4-yl)indole (39%). ¹H NMR (400 MHz, Chloroform-d) δ 8.75 (d, J=5.4 Hz, 1H), 7.44 (dt, J=9.0, 0.6 Hz, 1H), 7.33-7.23 (m, 1H), 7.06 (d, J=2.4 Hz, 1H), 6.83 (dd, J=8.9, 2.5 Hz, 1H), 6.49 (t, J=0.8 Hz, 1H), 3.87 (s, 3H), 3.62 (pd, J=6.8, 0.9 Hz, 1H), 2.81 (d, J=0.5 Hz, 3H), 1.27 (d, J=6.8 Hz, 6H). ESI-MS m/z calc. 281.2, found 282.0 (M+1)⁺.

### Preparation of S24

### Synthesis of 5-(benzyloxy)-6-fluoro-1-(4-fluoro-3-methylphenyl)-2-isopropyl-1H-indole (S24)

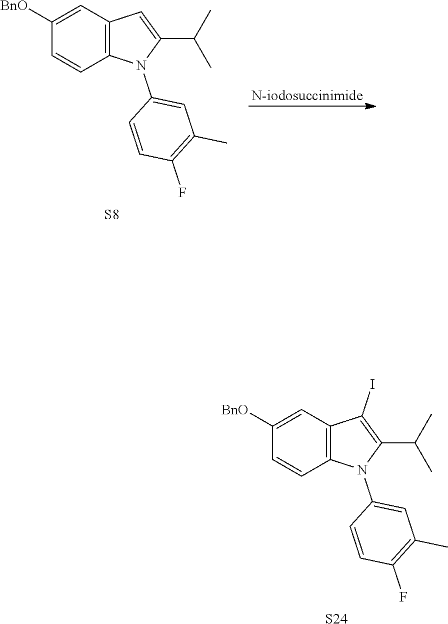 [SMILES: Cc1cc(-n2c(C(C)C)cc3cc(OCc4ccccc4)ccc32)ccc1F ; "S8" ; Cc1cc(-n2c(C(C)C)c(I)c3cc(OCc4ccccc4)ccc32)ccc1F ; "S24"]

To a cold (0° C.) of 5-benzyloxy-1-(4-fluoro-3-methyl-phenyl)-2-isopropyl-indole (4.00 g, 10.50 mmol) in CH₂Cl₂ (70 mL) was added N-iodosuccinimide (2.98 g, 12.58 mmol). The solution was stirred at 0° C. for 2.5 hr. The mixture was washed with aqueous saturated NaHCO₃, 1N Na₂S203 solution, dried over Na₂SO₄, filtered and concentrated in vacuo. The resulting residue was purified by silica gel chromatography (80 g ISCO column) using 0-30% EtOAc/heptanes gradient to afford desired product. 5-(benzyloxy)-6-fluoro-1-(4-fluoro-3-methylphenyl)-2-isopropyl-1H-indole (87%). ¹H NMR (400 MHz, Chloroform-d) δ 7.52-7.47 (m, 2H), 7.43-7.36 (m, 2H), 7.36-7.29 (m, 1H), 7.18-7.06 (m, 3H), 7.01 (dd, J=2.4, 0.5 Hz, 1H), 6.84 (dd, J=8.8, 2.4 Hz, 1H), 6.74 (dd, J=8.8, 0.5 Hz, 1H), 5.14 (s, 2H), 3.13-3.01 (m, 1H), 2.34 (d, J=2.1 Hz, 3H), 1.34 (dd, J=7.2, 3.2 Hz, 6H). ESI-MS m/z calc. 499.08, found 499.59 (M+1)⁺.

Compounds S25-S26 (Table 5) were made by a similar method to S24 from the appropriate indole intermediate.

TABLE 5
Structure and physicochemical data for compounds S25-S29

| | | Derived from | ¹H NMR; LCMS m/z |
|---|---|---|---|
| Intermediate | Structure | intermediate | [M + H]⁺ |
| S25 | 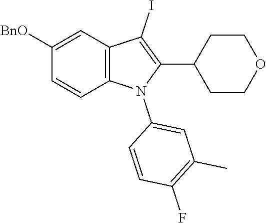 | S8 | ¹H NMR (400 MHz, Chloroform-d) δ 7.54- 7.47 (m, 2H), 7.46- 7.30 (m, 4H), 7.23- 7.06 (m, 4H), 6.94- 6.77 (m, 2H), 6.38 (d, J = 0.8 Hz, 1H), 5.14 (s, 2H), 4.00 (ddd, J = 11.8, 4.6, 2.0 Hz, 2H), 3.37 (td, J = 11.8, 2.4 Hz, 2H), 2.85-2.72 (m, 1H), 2.38 (d, J = 2.0 Hz, 3H), 1.82 (d, J = 4.2 Hz, 1H). LCMS m/z 416.8 [M + H]⁺ |
| S26 | 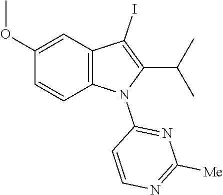 [SMILES: COc1ccc2c(c1)c(I)c(C(C)C)n2-c1ccnc(C)n1] | S22 | ¹H NMR (400 MHz, Chloroform-d) δ 8.82 (d, J = 5.4 Hz, 1H), 7.27 (d, J = 0.5 Hz, 1H), 7.19 (dd, J= 5.4, 0.6 Hz, 1H), 6.94 (d, J = 2.5 Hz, 1H), 6.86 (dd, J = 8.9, 2.5 Hz, 1H), 3.39 (hept, J = 7.0 Hz, 1H), 2.89- 2.73 (m, 3H), 1.50 (d, J = 7.1 Hz, 6H). |
| S27 | 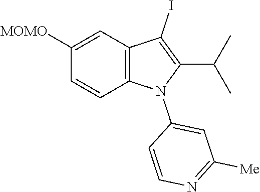 | | ¹H NMR (400 MHz, Chloroform-d) δ 8.71 (dd, J = 5.2, 0.7 Hz, 1H), 7.15 (td, J = 2.2, 0.6 Hz, 1H), 7.09 (ddd, J = 5.3, 2.0, 0.7 Hz, 1H), 6.98-6.82 (m, 2H), 5.26 (s, 2H), 3.55 (s, 3H), 3.11 (p, J = 7.2 Hz, 1H), 2.70 (s, 3H), 1.43 (d, J = 7.2 Hz, 6H), 1.37-1.21 (m, 1H), 0.97-0.81 (m, 1H). LCMS m/z 437.0 [M + H]⁺. |
| S28 | 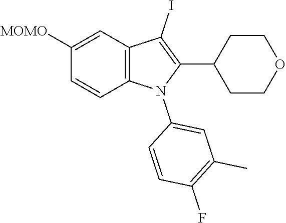 | | ¹H NMR (400 MHz, Chloroform-d) δ 6.99- 6.80 (m, 4H), 6.66 (dd, J = 8.8, 2.4 Hz, 1H), 6.51 (dd, J = 8.8, 0.5 Hz, 1H), 5.00 (s, 2H), 3.78 (dd, J = 11.6, 4.3 Hz, 2H), 3.30 (s, 3H), 3.11 (td, J = 11.9, 2.1 Hz, 2H), 2.82-2.63 (m, 1H), 2.31-2.03 (m, 5H). |
| S29 | 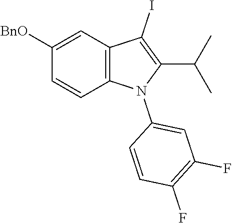 [SMILES: CC(C)c1c(I)c2cc(OCc3ccccc3)ccc2n1-c1ccc(F)c(F)c1] | S10 | LCMS m/z 504.0 [M + H]⁺. |

### Preparation S30 and S31

### Synthesis of 6-bromo-1-(4-fluoro-3-methylphenyl)-5-hydroxy-2-methyl-1H-indole-3-carbonitrile (S30) and 7-bromo-1-(4-fluoro-3-methylphenyl)-5-hydroxy-2-methyl-1H-indole-3-carbonitrile

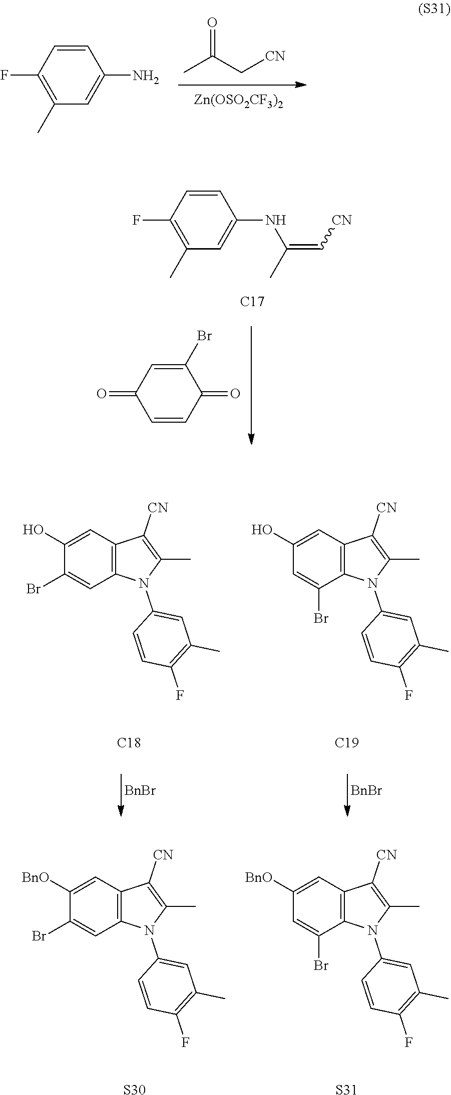

### Step 1. Synthesis of 3-((4-fluoro-3-methylphenyl)amino)but-2-enenitrile (C17)

To a solution of 3-oxobutanenitrile (4.93 g, 59.33 mmol) and 4-fluoro-3-methyl-aniline (7.42 g, 59.29 mmol) was added zinc trifluoromethanesulfonate (1.08 g, 2.97 mmol). The reaction mixture was stirred overnight at room temperature at which point the mixture solidified. The solid was dissolved in CH₂Cl₂ and purified by silica gel chromatography (330 g ISCO column) using 0-100% CH₂Cl₂/heptanes gradient to afford 7.9 g of product as a likely mixture of E and Z isomers. (Z)isomer: (Z)-3-(4-fluoro-3-methyl-anilino)but-2-enenitrile (68%). ¹H NMR (400 MHz, Chloroform-d) δ 7.06-6.83 (m, 3H), 5.70 (s, 1H), 4.21 (s, 1H), 2.26 (d, J=2.1 Hz, 3H), 2.24 (s, 3H). ESI-MS m/z calc. 190.09, found 191.29 (M+1)⁺.

### Step 2. Synthesis of 6-bromo-1-(4-fluoro-3-methylphenyl)-5-hydroxy-2-methyl-1H-indole-3-carbonitrile (C18) and 7-bromo-1-(4-fluoro-3-methylphenyl)-5-hydroxy-2-methyl-1H-indole-3-carbonitrile (C19)

To a refluxing solution of 2-bromo-1,4-benzoquinone (9.07 g, 43.65 mmol) and diiodozinc (1.33 g, 4.17 mmol) in CH₂Cl₂ (120 mL) was added dropwise a solution of 3-(4-fluoro-3-methyl-anilino)-but-2-enenitrile C17 (7.91 g, 41.58 mmol) in CH₂Cl₂ (33 mL). The mixture was heated at reflux for 1 hour and then cooled to room temperature. Divided sample into two lots for purification. The resulting residue was purified by silica gel chromatography using 0-10% EtOAc/CH₂Cl₂ gradient to afford 1.5 g of first product. 6-bromo-1-(4-fluoro-3-methyl-phenyl)-5-hydroxy-2-methyl-indole-3-carbonitrile (20%). ¹H NMR (400 MHz, DMSO-d6) δ 10.15 (s, 1H), 7.54-7.45 (m, 1H), 7.45-7.36 (m, 2H), 7.16 (s, 1H), 7.10 (s, 1H), 2.35 (s, 3H), 2.34-2.27 (m, 3H). ESI-MS m/z calc. 358.01, found 359.02 (M+1)⁺. Second product isolated. 7-bromo-1-(4-fluoro-3-methyl-phenyl)-5-hydroxy-2-methyl-indole-3-carbonitrile (1.04 g, 14%). ¹H NMR (400 MHz, DMSO-d6) δ 9.69 (s, 1H), 7.48-7.38 (m, 1H), 7.38-7.29 (m, 2H), 7.00-6.86 (m, 2H), 2.34-2.26 (m, 3H), 2.23 (s, 3H). ESI-MS m/z calc. 358.01, found 359.07 (M+1)⁺.

### Step 3a. Synthesis 6-bromo-1-(4-fluoro-3-methylphenyl)-5-hydroxy-2-methyl-1H-indole-3-carbonitrile (S30)

To a suspension of 6-bromo-1-(4-fluoro-3-methyl-phenyl)-5-hydroxy-2-methyl-indole-3-carbonitrile C18 (0.94 g, 2.57 mmol) and K₂CO₃ (0.71 g, 5.15 mmol) in DMF (6 mL) was added benzylbromide (0.35 mL, 2.94 mmol). The reaction mixture was heated to 70° for 4 hours. The mixture was cooled to room temperature, diluted with water and stirred for 30 minutes. Filtered brown precipitate. Triturated with heptanes and filtered. The brown solid was purified by silica gel chromatography (120 g ISCO column) using CH₂Cl to afford 1.12 g product. 5-benzyloxy-6-bromo-1-(4-fluoro-3-methyl-phenyl)-2-methyl-indole-3-carbonitrile (95%). ¹H NMR (400 MHz, DMSO-d6) δ 7.55-7.48 (m, 3H), 7.46-7.37 (m, 5H), 7.37-7.30 (m, 1H), 7.28 (s, 1H), 5.31 (s, 2H), 2.37 (s, 3H), 2.32 (d, J=1.4 Hz, 3H). ESI-MS m/z calc. 448.06, found 449.1 (M+1)⁺.

### Step 3b. Synthesis 7-bromo-1-(4-fluoro-3-methylphenyl)-5-hydroxy-2-methyl-1H-indole-3-carbonitrile (S31)

To a suspension of 7-bromo-1-(4-fluoro-3-methyl-phenyl)-5-hydroxy-2-methyl-indole-3-carbonitrile C19 (0.54 g, 1.47 mmol) and K₂CO₃ (0.61 g, 4.41 mmol) in DMF (3.5 mL) was added benzylbromide (0.35 mL, 2.94 mmol). The reaction mixture was heated to 70° for 1 hour. The mixture was cooled to room temperature, diluted with water and EtOAc. The organic phase was washed dried (MgSO₄), filtered, and concentrated in vacuo. The resulting residue was purified by silica gel chromatography (40 g ISCO column) using 0-50% CH₂Cl₂/heptanes gradient to afford 620 mg product. 5-benzyloxy-7-bromo-1-(4-fluoro-3-methyl-phenyl)-2-methyl-indole-3-carbonitrile (94%). ¹H NMR (400 MHz, Chloroform-d) δ 7.51-7.31 (m, 5H), 7.19 (d, J=2.3 Hz, 1H), 7.18-7.05 (m, 4H), 5.11 (s, 2H), 2.35 (d, J=2.1 Hz, 3H), 2.30 (s, 3H).

### Preparation of S32

### Synthesis of 5-(benzyloxy)-2-bromo-1-(4-fluorophenyl)-1H-indole-3-carbonitrile (S32)

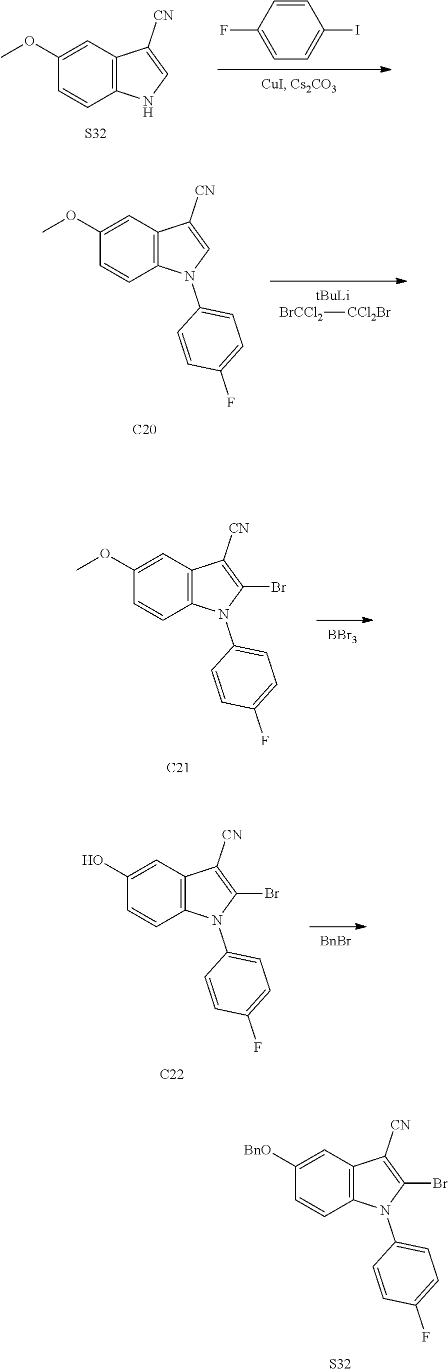

### Step 1. Synthesis of 1-(4-fluorophenyl)-5-methoxy-1H-indole-3-carbonitrile (C20)

To a solution of 5-methoxy-1H-indole-3-carbonitrile S32 (1.25 g, 7.28 mmol), 1-fluoro-4-iodo-benzene (1.76 g, 7.93 mmol) in DMF (12 mL) purged with nitrogen was added iodocopper (0.28 g, 1.45 mmol) and Cs₂CO₃ (3.56 g, 10.92 mmol). The reaction was sealed and heated at 120° C. for 15 hours. The mixture was diluted with water and extracted three times with EtOAc. The combined organic phases were washed with water, dried (MgSO₄), filtered, and concentrated in vacuo. The resulting residue was purified by silica gel chromatography (40 g ISCO column) using 0-50% EtOAc/heptanes gradient to afford 1.03 g product. 1-(4-fluorophenyl)-5-methoxy-indole-3-carbonitrile (53%). ¹H NMR (300 MHz, Chloroform-d) δ 7.72 (d, J=1.9 Hz, 1H), 7.58-7.40 (m, 2H), 7.38-7.11 (m, 4H), 6.99 (dd, J=9.1, 2.4 Hz, 1H), 3.93 (d, J=2.0 Hz, 3H). ESI-MS m/z calc. 266.08, found 267.12 (M+1)⁺.

### Step 2. Synthesis of 2-bromo-1-(4-fluorophenyl)-5-methoxy-1H-indole-3-carbonitrile (C21)

To a cold (−10° C.) solution of 1-(4-fluorophenyl)-5-methoxy-indole-3-carbonitrile C20 (12.05 g, 45.25 mmol) in THE (280 mL) was added dropwise a solution of tert-butyllithium (31 mL of 1.7 M solution in pentane, 52.70 mmol). After 1 hour, a solution of 1,2-dibromo-1,1,2,2-tetrachloro-ethane (19.0 g, 58.0 mmol) in THE (60 mL) was added dropwise. After 1 hour, the cooling bath was removed and the mixture was stirred at room temperature for 2 hours. The mixture was diluted with water and extracted three times with EtOAc. The combined organic phases were dried (MgSO₄), filtered, and concentrated in vacuo. The resulting residue was purified by silica gel chromatography (220 g ISCO column) using 0-20% EtOAc/heptanes gradient to afford 14.7 g of product. 2-bromo-1-(4-fluorophenyl)-5-methoxy-indole-3-carbonitrile (94%). ESI-MS m/z calc. 344.0, found 345.1 (M+1)⁺.

### Step 3. Synthesis of 2-bromo-1-(4-fluorophenyl)-5-hydroxy-1H-indole-3-carbonitrile (C22)

To a cold (0° C.) solution of 2-bromo-1-(4-fluorophenyl)-5-methoxy-indole-3-carbonitrile C21 (13.2 g, 38.2 mmol) in CH₂Cl₂ (250 mL) was added tribromoborane (90 mL of 1 M solution in CH₂Cl₂, 90.0 mmol). After 90 minutes, the cooling bath was removed and the mixture was stirred at room temperature for 1 h. Water was added carefully. The mixture was extracted with three times with CH₂Cl₂. There was white solid in the aqueous phase and collected through filtration. The combined organic phases were evaporated. The residue and the solid were dissolved in 20% MeOH/CH₂Cl₂ and the mixture was purified by silica gel chromatography (220 g ISCO column) using a 0-4% MeOH/CH₂Cl₂ gradient to afford 11.9 g of product. 2-bromo-1-(4-fluorophenyl)-5-hydroxy-indole-3-carbonitrile (94%). ¹H NMR (300 MHz, DMSO-d6) δ 9.57 (s, 1H), 7.83-7.58 (m, 2H), 7.57-7.34 (m, 2H), 6.95 (dd, J=5.4, 3.1 Hz, 2H), 6.80 (dd, J=9.0, 2.3 Hz, 1H). ESI-MS m/z calc. 329.98, found 330.65 (M+1)⁺.

### Step 4. Synthesis of 5-(benzyloxy)-2-bromo-1-(4-fluorophenyl)-1H-indole-3-carbonitrile (S32)

To a solution of 2-bromo-1-(4-fluorophenyl)-5-hydroxy-indole-3-carbonitrile C22 (1.10 g, 3.32 mmol) and CS₂CO₃ (3.50 g, 10.74 mmol) in acetone (25 mL) was added benzyl bromide (0.75 mL, 6.31 mmol). The reaction mixture was heated at 70° C. at room temperature for 18 hours. The solvent was removed under reduced pressure and the resulting residue was dissolved in EtOAc (10 mL) and washed with aqueous saturated NaHCO₃ solution. The organic phase was dried (MgSO₄), filtered, and concentrated in vacuo. The resulting residue was purified by silica gel chromatography using 0-50% EtOAc/heptanes gradient to afford 870 mg of product. 5-benzyloxy-2-bromo-1-(4-fluorophenyl)indole-3-carbonitrile (60%). ESI-MS m/z calc. 420.02, found 420.98 (M+1)⁺.

### Preparation of S33

### 2-bromo-1-(4-fluorophenyl)-5-(methoxymethoxy)-1H-indole-3-carbonitrile (S33)

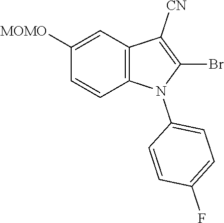

S33 is made by a similar method to S32 using OMOM as replacement for OBn. 2-bromo-1-(4-fluorophenyl)-5-(methoxymethoxy)-1H-indole-3-carbonitrile. ¹H NMR (300 MHz, DMSO-d6) δ 7.77-7.60 (m, 2H), 7.58-7.40 (m, 2H), 7.31 (dd, J=2.1, 0.7 Hz, 1H), 7.11-6.89 (m, 2H), 5.27 (s, 3H), 3.40 (s, 3H). ESI-MS m/z calc. 374.00, found 375.01 (M+1)⁺.

### Preparation S34

### 2-bromo-1-(4-fluorophenyl)-5-methoxy-1H-indole-3-carbonitrile (S34)

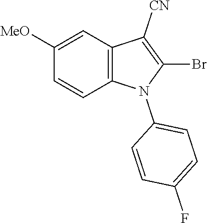 [SMILES: COc1ccc2c(c1)c(C#N)c(Br)n2-c1ccc(F)cc1]

S34 is made by a similar method to S32 using OMe as replacement for OBn. 2-bromo-1-(4-fluorophenyl)-5-methoxy-1H-indole-3-carbonitrile. ESI-MS m/z calc. 344.0, found 345.1 (M+1)⁺.

### Preparation of S35

### 5-(benzyloxy)-2-bromo-1-phenyl-1H-indole-3-carbonitrile (S35)

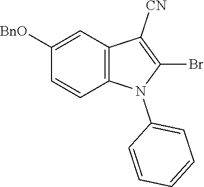 [SMILES: N#Cc1c(Br)n(-c2ccccc2)c2ccc(OCc3ccccc3)cc12]

S35 is made by a similar method to S30 using iodobenzene. 5-(benzyloxy)-2-bromo-1-phenyl-1H-indole-3-carbonitrile. ESI-MS m/z calc. 402.04, found 403.09 (M+1)⁺.

### Preparation of S36

### Synthesis of 2-(J-(4-fluorophenyl)-5-methoxy-2-methyl-1H-indol-3-yl)acetonitrile (S36)

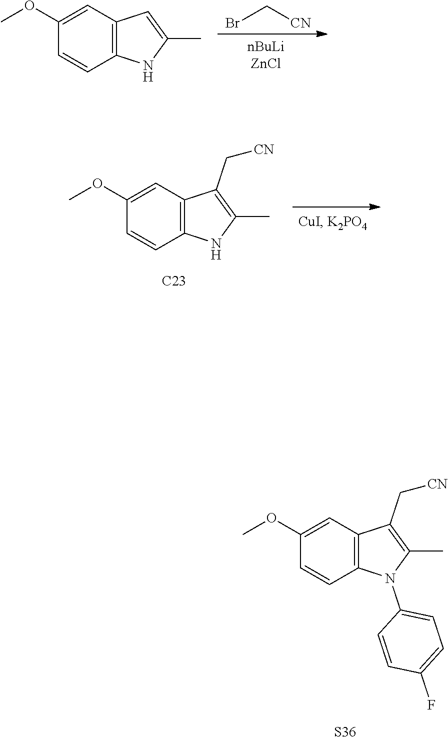

### Step 1. Synthesis of 2-(5-methoxy-2-methyl-1H-indol-3-yl)acetonitrile (C23)

To a cold (0° C.) solution of 5-methoxy-2-methyl-1H-indole (6.45 g, 40.01 mmol) in THE (80 mL) was added dropwise n-butyllithium (16 mL of 2.5 M solution in hexanes, 40 mmol) while the internal temperature was kept below 10° C. with an ice/ethanol bath. After 0.25 hours, zinc chloride (80 mL of 0.5 M in THF, 40 mmol) was added dropwise, while internal temperature was maintained between 0-2° C. The cooling bath was removed, and the mixture was stirred for 2 h and then concentrated at reduced pressure to a give a wax which was dissolved in toluene (80 mL). To this solution was added bromo acetonitrile (2.75 mL, 40.01 mmol) and the mixture was stirred for 24 hours at room temperature. Additional bromo acetonitrile (2.75 mL, 40.01 mmol) was added and the mixture was stirred for an additional 1 hour. The reaction mixture was quenched with 1 M HCl (30 mL) and the layers were separated. The organic phase was washed with brine. The aqueous layer was extracted once more with EtOAc, then washed once with brine. The combined organic phases were dried over Na₂SO₄, filtered and concentrated in vacuo. The resulting residue was purified by silica gel chromatography using 0-5% EtOAc/CH₂Cl₂ gradient to afford 4.1 g of product. 2-(5-methoxy-2-methyl-1H-indol-3-yl)acetonitrile (51%). ¹H NMR (400 MHz, DMSO-d6) δ 10.86 (s, 1H), 7.17 (d, J=8.7 Hz, 1H), 7.02 (d, J=2.4 Hz, 1H), 6.68 (dd, J=8.7, 2.4 Hz, 1H), 3.93 (s, 2H), 3.76 (s, 3H), 2.35 (s, 3H). ESI-MS m/z calc. 200.1, found 201.0 (M+1)⁺.

### Step 2. Synthesis of 2-(1-(4-fluorophenyl)-5-methoxy-2-methyl-1H-indol-3-yl)acetonitrile (S36)

To a suspension of 2-(5-methoxy-2-methyl-1H-indol-3-yl)acetonitrile C23 (1.32 g, 6.59 mmol) in toluene (13.2 mL) degassed for 10 minutes with nitrogen was added K₃PO₄ (4.2 g, 19.8 mmol), iodocopper (0.75 g, 3.96 mmol), N,N′-dimethylethane-1,2-diamine (0.42 mL, 3.956 mmol) and 1-fluoro-4-iodo-benzene (approximately 2.93 g, 13.18 mmol). The pressure flask was sealed with a screw cap and the reaction mixture was heated at 110° C. for 16 h. The reaction mixture was allowed to cool to room temperature and filtered through a plug of celite, with further washing with CH₂Cl₂. The filtrate was concentrated to a dark oil under reduced pressure and the crude material was purified by silica gel chromatography using 0-20% EtOAc/CH₂Cl₂ gradient to afford 845 mg of product. 2-[1-(4-fluorophenyl)-5-methoxy-2-methyl-indol-3-yl]acetonitrile (44%). ESI-MS m/z calc. 294.1, found 295.2 (M+1)⁺.

### Preparation of S37

### Synthesis of 1-(4-fluoro-3-methylphenyl)-3-iodo-5-methoxy-1H-indole-2-carbonitrile (S37)

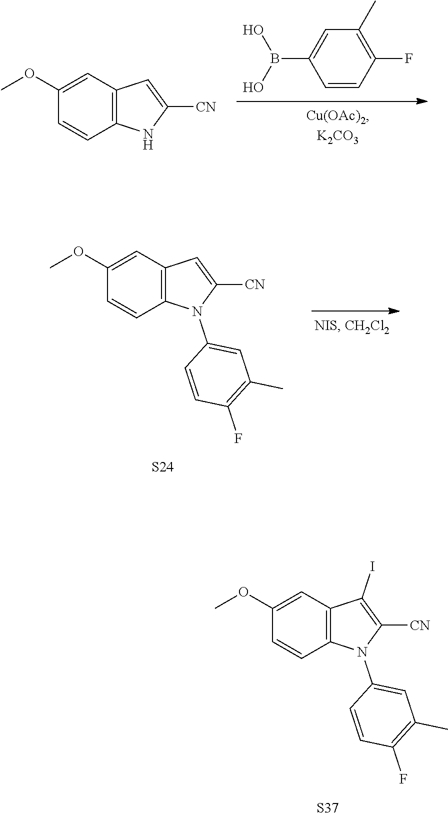

### Step 1. Synthesis of 1-(4-fluoro-3-methylphenyl)-5-methoxy-1H-indole-2-carbonitrile (C24)

To a solution of 5-methoxy-1H-indole-2-carbonitrile (0.133 g, 0.704 mmol), (4-fluoro-3-methyl-phenyl)boronic acid (0.219 g, 1.423 mmol), copper (II) acetate (0.270 g, 1.487 mmol), potassium carbonate (0.225 g, 1.628 mmol) in dimethyl sulfoxide (2 mL) was added 3 angstrom molecular sieves (0.235 g). The reaction mixture was stirred open to air at room temperature overnight. The mixture was diluted with water and extracted twice with ethyl acetate. The combined organic phases were washed twice with water, brine, dried over sodium sulfate, filtered and concentrated under reduced pressure. The crude residue was purified by reverse phase flash chromatography (RF ISCO, C18 column, 30 g) eluting with CH₃CN/water (0-100%, 0.1% TFA) to afford 88 mg of product. 1-(4-fluoro-3-methylphenyl)-3-iodo-5-methoxy-1H-indole-2-carbonitrile (44%). ¹H NMR (400 MHz, DMSO-d6) δ 7.63-7.52 (m, 2H), 7.50-7.37 (m, 2H), 7.28-7.17 (m, 2H), 7.06 (dd, J=9.1, 2.5 Hz, 1H), 3.80 (s, 3H), 2.34 (d, J=2.1 Hz, 3H). ESI-MS m/z calc. 280.10, found 281.47 (M+1)⁺.

### Step 2. Synthesis of 1-(4-fluoro-3-methylphenyl)-3-iodo-5-methoxy-1H-indole-2-carbonitrile (S37)

To a cold (0° C.) solution of 1-(4-fluoro-3-methyl-phenyl)-5-methoxy-indole-2-carbonitrile (0.088 g, 0.306 mmol) in dichloromethane (1.5 mL) was added N-Iodosuccinimide (0.077 g, 0.342 mmol). The reaction mixture was stirred for 1 hour at 0° C. The ice bath was removed and the mixture was warmed to room temperature and stirred for 36 hours. The reaction was quenched with water and extracted twice with CH₂Cl₂. The combined organic phases were washed with 1N sodium thiosulfate, passed through a phase separator, and resulting filtrate concentrated under reduced pressure. The resulting residue was purified by silica gel chromatography (24 g ISCO column) using 0-100% EtOAc/CH₂Cl₂ gradient to afford 52 mg of product. 1-(4-fluoro-3-methyl-phenyl)-3-iodo-5-methoxy-indole-2-carbonitrile (39%). ¹H NMR (400 MHz, Chloroform-d) δ 7.33-7.25 (m, 2H), 7.22 (t, J=8.7 Hz, 1H), 7.17 (dd, J=9.1, 0.6 Hz, 1H), 7.08 (dd, J=9.1, 2.4 Hz, 1H), 6.89 (dd, J=2.3, 0.5 Hz, 1H), 3.94 (s, 3H), 2.40 (d, J=2.1 Hz, 3H). ESI-MS m/z calc. 406.0, found 407.3 (M+1)⁺.

### Preparation of S38

### Synthesis of 5-(benzyloxy)-1-(4-fluoro-3-methylphenyl)-2-(tetrahydrofuran-2-yl)-3-vinyl-1H-indole (S38)

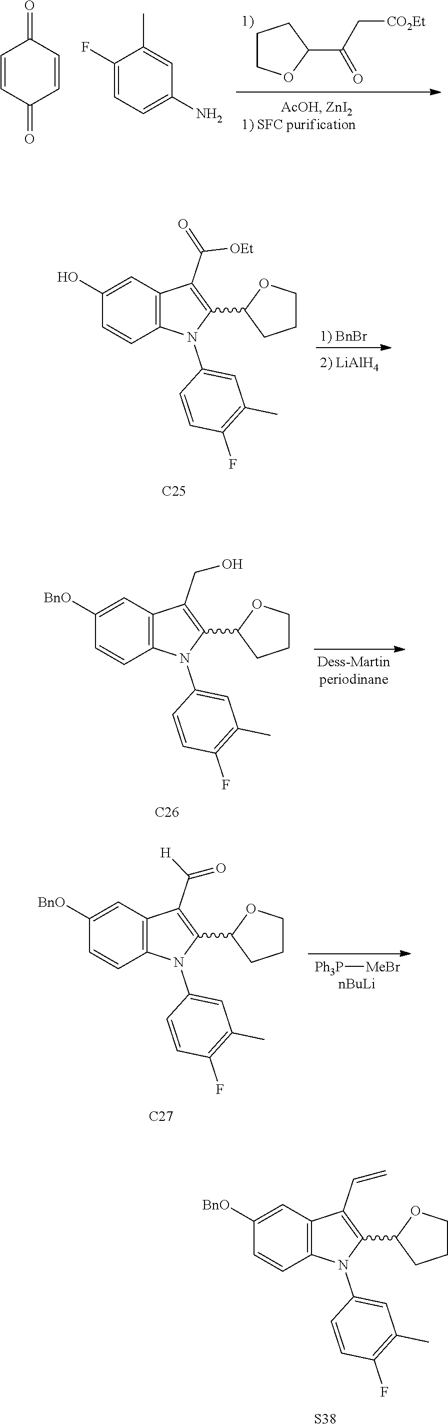

### Step 1. Synthesis of ethyl 1-(4-fluoro-3-methylphenyl)-5-hydroxy-2-(tetrahydrofuran-2-yl)-1H-indole-3-carboxylate (C25)

A suspension of 4-fluoro-3-methyl-aniline (2.00 g, 15.98 mmol) and ethyl 3-oxo-3-tetrahydrofuran-2-yl-propanoate (approximately 2.97 g, 15.98 mmol) in AcOH (0.09 mL, 1.59 mmol) in a sealed Teflon septa vial was heated at 90° C. for 16 hours. The reaction mixture was cooled to room temperature diluted with CH₂Cl₂ and then concentrated under reduced pressure, and this was repeated twice more. The residue was then further dried under high vacuum for 1 h, then dissolved in anhydrous CH₂Cl₂ (62 mL) under a nitrogen atmosphere upon which 1,4-benzoquinone (1.73 g, 15.98 mmol) was added followed by diiodozinc (0.51 g, 1.59 mmol) and the reaction was then heated at reflux for 24 h under a nitrogen atmosphere. The reaction mixture was cooled to room temperature, filtered and the filtrate was concentrated under reduced pressure. The resulting residue was purified by silica gel chromatography using 0-20% EtOAc/heptanes gradient. The desired fractions were pooled and concentrated in vacuo and the solid was triturated with Et₂O/hexanes to afford 350 mg of product. Ethyl 1-(4-fluoro-3-methyl-phenyl)-5-hydroxy-2-tetrahydrofuran-2-yl-indole-3-carboxylate (5%). SFC chiral chromatography afforded separation of the enantiomers. ¹H NMR (400 MHz, DMSO-d6) δ 9.07 (s, 1H), 7.42 (d, J=2.2 Hz, 1H), 7.41-7.19 (m, 3H), 6.66 (dd, J=8.9, 1.8 Hz, 1H), 6.58 (d, J=8.5 Hz, 1H), 5.79-5.70 (m, 1H), 4.31 (q, J=7.1 Hz, 2H), 3.55-3.47 (m, 1H), 3.06-2.97 (m, 1H), 2.30 (s, 3H), 2.27-2.17 (m, 1H), 1.98-1.71 (m, 2H), 1.61-1.50 (m, 1H), 1.37 (t, J=7.1 Hz, 3H). ESI-MS m/z calc. 383.1533, found 384.5 (M+1)⁺.

### Step 2. Synthesis of (5-(benzyloxy)-1-(4-fluoro-3-methylphenyl)-2-(tetrahydrofuran-2-yl)-1H-indol-3-yl)methanol (C26)

To a solution of ethyl 1-(4-fluoro-3-methyl-phenyl)-5-hydroxy-2-[(2R)-tetrahydrofuran-2-yl]indole-3-carboxylate C25 (0.63 g, 1.65 mmol) in DMF (6.5 mL) was added K₂CO₃ (0.71 g, 5.10 mmol) and the reaction was cooled to 0° C. Bromomethylbenzene (0.26 mL, 2.14 mmol) was added slowly under an atmosphere of nitrogen. The reaction mixture was gradually warmed to room temperature and stirred for 4 hours. The mixture was diluted with water and diethyl ether. The aqueous phase was washed with diethyl ether. The organic phase was dried (MgSO₄), filtered, and concentrated in vacuo. The resulting residue was purified by silica gel chromatography using 0-60% EtOAc/heptanes gradient to afford 725 mg of product. Ethyl 5-benzyloxy-1-(4-fluoro-3-methyl-phenyl)-2-[(2R)-tetrahydrofuran-2-yl]indole-3-carboxylate (93%). ¹H NMR (400 MHz, DMSO-d6) δ 7.59 (d, J=2.5 Hz, 1H), 7.52-7.19 (m, 8H), 6.95-6.83 (m, 1H), 6.70 (dd, J=8.9, 2.0 Hz, 1H), 5.76 (q, J=8.2 Hz, 1H), 5.16 (s, 2H), 4.31 (q, J=7.1 Hz, 2H), 3.52 (dt, J=7.9, 4.1 Hz, 1H), 3.00 (p, J=7.0 Hz, 1H), 2.37-2.16 (m, 4H), 1.92 (dt, J=12.1, 8.7 Hz, 1H), 1.79 (dt, J=20.1, 8.1 Hz, 1H), 1.56 (s, 1H), 1.35 (t, J=7.1 Hz, 3H). ESI-MS m/z calc. 473.20, found 474.37 (M+1)⁺.

To a solution of ethyl 5-benzyloxy-1-(4-fluoro-3-methyl-phenyl)-2-[(2R)-tetrahydrofuran-2-yl]indole-3-carboxylate (0.70 g, 1.48 mmol) in THE (18 mL) was added lithium aluminum hydride (1.5 mL of 1 M, 1.5 mmol). The reaction mixture was stirred at room temperature overnight. Rochelle salt and CH₂Cl₂ were added. The resulting residue was purified by silica gel chromatography (40 g ISCO column) using EtOAc/heptanes gradient to afford 533 mg of product. [5-benzyloxy-1-(4-fluoro-3-methyl-phenyl)-2-[(2R)-tetrahydrofuran-2-yl]indol-3-yl]methanol (84%). ESI-MS m/z calc. 431.19, found 430.78 (M+1)⁺.

### Step 3. Synthesis of 5-(benzyloxy)-1-(4-fluoro-3-methylphenyl)-2-(tetrahydrofuran-2-yl)-1H-indole-3-carbaldehyde (C27)

To cold (0° C.) solution of [5-benzyloxy-1-(4-fluoro-3-methyl-phenyl)-2-[(2R)-tetrahydrofuran-2-yl]indol-3-yl]methanol C₂₆ (0.44 g, 1.02 mmol) in CH₂Cl₂ (12 mL) was added a solution of (1,1-diacetoxy-3-oxo-1Î>>5,2-benziodoxol-1-yl) acetate (0.43 g, 1.02 mmol) in CH₂Cl₂ (12 mL). After 30 minutes, the mixture was diluted into 2N NaOH and CH₂Cl₂. The phases were separated by passing through a phase separator. The resulting residue was purified by silica gel chromatography using a EtOAc/heptanes gradient to afford 113 mg of product. 5-benzyloxy-1-(4-fluoro-3-methyl-phenyl)-2-[(2R)-tetrahydrofuran-2-yl]indole-3-carbaldehyde (18%). ¹H NMR (400 MHz, DMSO-d6) δ 10.37 (s, 1H), 7.87 (d, J=2.4 Hz, 1H), 7.59-7.24 (m, 8H), 6.94 (dd, J=9.0, 2.5 Hz, 1H), 6.83 (d, J=8.9 Hz, 1H), 5.15 (s, 2H), 4.99 (dt, J=12.7, 7.6 Hz, 1H), 3.86 (dq, J=13.5, 6.9 Hz, 1H), 3.69 (q, J=7.1 Hz, 1H), 2.32 (d, J=2.3 Hz, 3H), 2.17 (d, J=7.0 Hz, 1H), 2.04-1.79 (m, 3H). ESI-MS m/z calc. 429.17, found 430.31 (M+1)⁺.

### Step 4. Synthesis of 5-(benzyloxy)-1-(4-fluoro-3-methylphenyl)-2-(tetrahydrofuran-2-yl)-3-vinyl-1H-indole (S38)

n-BuLi (0.165 mL of 2.5 M, 0.413 mmol) was added to a cold (0° C.) solution of methyl-(triphenyl)phosphonium bromide (0.131 g, 0.367 mmol) in THE (2.4 mL) under nitrogen. The resulting yellow color solution was stirred at 0° C. for 2 hours and 5-benzyloxy-1-(4-fluoro-3-methyl-phenyl)-2-[(2R)-tetrahydrofuran-2-yl]indole-3-carbaldehyde C₂₇ (0.113 g, 0.182 mmol) in THE (0.6 mL) was added dropwise. The cooling bath was removed and the mixture was stirred at room temperature for 2 hours. The mixture was quenched with aqueous saturated NH₄Cl solution. The solvent was removed under reduced pressure and the crude product was dissolved in EtOAc (200 mL) and washed with brine. The organic phase dried over Na₂SO₄, filtered and concentrated under reduced pressure. The resulting residue was purified by chromatography on neutral alumina using EtOAc/heptanes to afford 79 mg of product. 5-benzyloxy-1-(4-fluoro-3-methyl-phenyl)-2-[(2R)-tetrahydrofuran-2-yl]-3-vinyl-indole (100%). ESI-MS m/z calc. 427.19, found 428.33 (M+1)⁺.

Compounds S39-S44 (Table 6) were prepared from the appropriate indole intermediate as described for the preparation of S38.

TABLE 6
Structure and physicochemical data for compounds S39-S44

| Intermediate | Structure | ¹H NMR; LCMS m/z [M + H]⁺ |
|---|---|---|
| S39 | 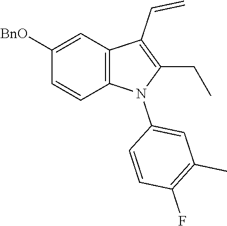 [SMILES: C=Cc1c(CC)n(-c2ccc(F)c(C)c2)c2ccc(OCc3ccccc3)cc12] | ¹H NMR (400 MHz, Chloroform-d) δ 7.51-7.44 (m, 2H), 7.35 (m, 3H), 7.18- 7.06 (m, 3H), 6.95-6.76 (m, 3H), 5.63 (d, J = 17.8 Hz, 1H), 5.23 (d, J = 11.6 Hz, 1H), 5.13 (s, 2H), 5.05-4.93 (m, 1H), 2.70 (q, J = 7.2, 6.8 Hz, 2H), 2.34 (s, 3H), 1.04 (t, J = 7.5 Hz, 3H). LCMS m/z 385.7 [M + H]⁺ |
| S40 | 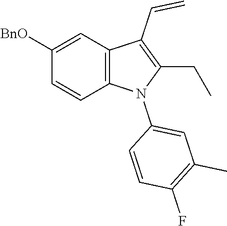 | LCMS m/z 401.0 [M + H]⁺ |
| S41 | 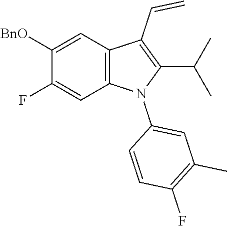 [SMILES: C=Cc1c(C(C)C)n(-c2ccc(F)c(C)c2)c2cc(F)c(OCc3ccccc3)cc12] | ¹H NMR (400 MHz, Chloroform-d) δ 7.52 (dd, J = 11.0, 7.6 Hz, 3H), 7.42 (dd, J = 8.3, 6.6 Hz, 2H), 7.36 (d, J = 7.2 Hz, 1H), 7.23-6.96 (m, 4H), 6.65 (d, J = 11.3 Hz, 1H), 5.56 (dd, J = 17.7, 1.6 Hz, 1H), 5.29 (dd, J = 11.3, 1.6 Hz, 1H), 5.20 (s, 2H), 3.03 (m, 1H), 2.38 (d, J = 2.0 Hz, 3H), 1.32 (dd, J = 7.2, 2.1 Hz, 6H). LCMS m/z 417.6 [M + H]⁺ |
| S42 | 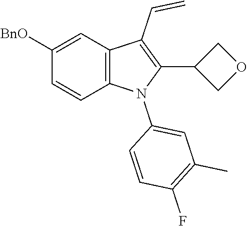 | ¹H NMR (400 MHz, Chloroform-d) δ 7.16-6.98 (m, 4H), 6.90 (d, J = 8.7 Hz, 1H), 6.71 (dd, J = 8.7, 2.2 Hz, 1H), 4.98 (dd, J = 7.2, 5.2 Hz, 1H), 4.91-4.78 (m, 1H), 4.75-4.49 (m, 3H), 2.60 (m, 1H), 2.33 (s, 3H), 1.89 (dt, J = 8.5, 4.6 Hz, 1H), 1.68 (dt, J = 8.9, 4.4 Hz, 1H), 1.39-1.30 (m, 1H). LCMS m/z 381.6 [M + H]⁺ |
| S43 | 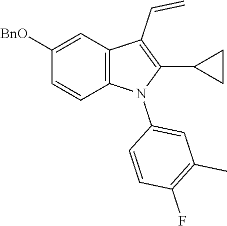 [SMILES: C=Cc1c(C2CC2)n(-c2ccc(F)c(C)c2)c2ccc(OCc3ccccc3)cc12] | ¹H NMR (400 MHz, Chloroform-d) δ 7.59-7.29 (m, 6H), 7.24-7.08 (m, 4H), 6.99 (d, J = 8.7 Hz, 1H), 6.87 (dd, J = 9.0, 2.6 Hz, 1H), 5.64 (dd, J = 17.9, 1.8 Hz, 1H), 5.34-5.23 (m, 3H), 5.13 (s, 2H), 2.46-2.30 (m, 3H), 1.88-1.74 (m, 1H), 0.84-0.73 (m, 2H), 0.61-0.46 (m, 2H). LCMS m/z 398.7 [M + H]⁺. |
| S44 | 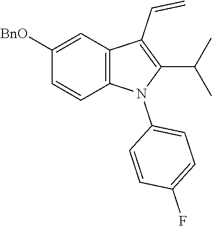 [SMILES: C=Cc1c(C(C)C)n(-c2ccc(F)cc2)c2ccc(OCc3ccccc3)cc12] | ¹H NMR (400 MHz, Chloroform-d) δ 7.55-7.45 (m, 4H), 7.41-7.26 (m, 8H), 7.10 (dd, J = 17.7, 11.5 Hz, 1H), 6.84 (dd, J = 8.8, 2.4 Hz, 1H), 6.73 (dt, J = 8.8, 0.7 Hz, 1H), 5.63 (dd, J = 17.7, 1.7 Hz, 1H), 5.37-5.23 (m, 2H), 5.12 (s, 2H), 2.96 (td, J = 7.2, 1.0 Hz, 1H), 1.35 (d, J = 7.2 Hz, 3H), 1.27 (d, J = 7.2 Hz, 3H). LCMS m/z 386.3 [M + H]⁺. |

### Compounds 1 and 2

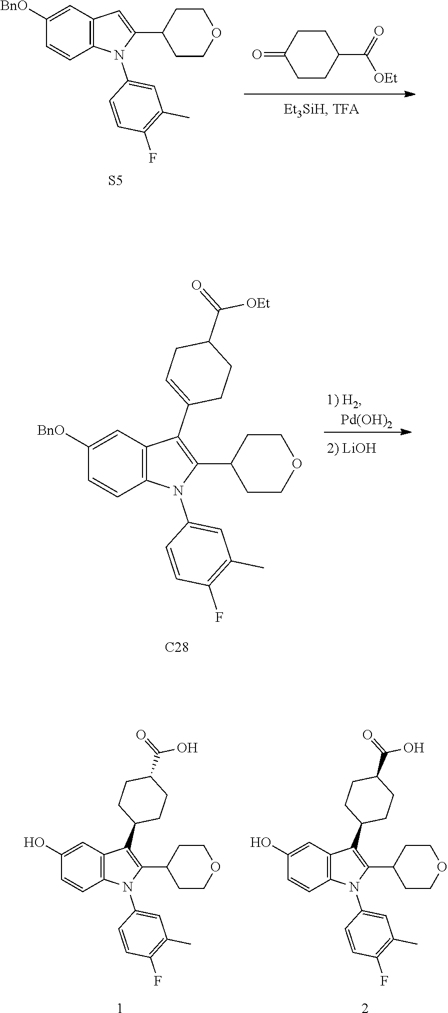

### Step 1. Synthesis of ethyl 4-(5-(benzyloxy)-1-(4-fluoro-3-methylphenyl)-2-(tetrahydro-2H-pyran-4-yl)-1H-indol-3-yl)cyclohex-3-ene-1-carboxylate (C28)

A solution of 5-benzyloxy-1-(4-fluoro-3-methyl-phenyl)-2-tetrahydropyran-4-yl-indole S5 (0.35 g, 0.84 mmol), ethyl 4-oxocyclohexanecarboxylate (0.60 g, 3.53 mmol), trifluoroacetic acid (0.30 mL, 3.89 mmol) and triethylsilane (0.54 mL, 3.38 mmol) in CH₂Cl₂ (7 mL) was stirred at 50° C. for 3 days. The reaction mixture was washed with water and dried over Na₂SO₄. The solvent was removed under reduced pressure and crude product was purified by silica gel chromatography eluting with 0-50% EtOAc/heptane to afford 226 mg of product. Ethyl 4-[5-benzyloxy-1-(4-fluoro-3-methyl-phenyl)-2-tetrahydropyran-4-yl-indol-3-yl]cyclohex-3-ene-1-carboxylate (47%). Retention time: 0.9 minutes 1H NMR (400 MHz, Chloroform-d) δ 7.53-7.47 (m, 2H), 7.45-7.39 (m, 2H), 7.34 (d, J=7.3 Hz, 1H), 7.19-7.06 (m, 3H), 6.98 (d, J=2.3 Hz, 1H), 6.84 (dd, J=8.8, 2.4 Hz, 1H), 6.79-6.66 (m, 1H), 5.81 (s, 1H), 5.11 (s, 1H), 4.27-4.23 (m, 2H), 3.97 (d, J=11.4 Hz, 2H), 3.30 (t, J=12.0 Hz, 2H), 2.87-2.69 (m, 2H), 2.56-2.53 (m, 2H), 2.43 (m, 2H), 2.37 (d, J=2.0 Hz, 3H), 2.19-2.17 (m, 1H), 2.11-1.92 (m, 3H), 1.64-1.62 (m, 2H), 1.34 (t, J=7.1 Hz, 3H). ESI-MS m/z calc. 567.28, found 568.53 (M+1)⁺.

### Step 2. Synthesis of trans-4-(1-(4-fluoro-3-methylphenyl)-5-hydroxy-2-(tetrahydro-2H-pyran-4-yl)-1H-indol-3-yl)cyclohexane-1-carboxylic acid (1) and cis-4-(1-(4-fluoro-3-methylphenyl)-5-hydroxy-2-(tetrahydro-2H-pyran-4-yl)-1H-indol-3-yl)cyclohexane-1-carboxylic acid (2)

To a solution of ethyl 4-[5-benzyloxy-1-(4-fluoro-3-methyl-phenyl)-2-tetrahydropyran-4-yl-indol-3-yl]cyclohex-3-ene-1-carboxylate C28 (0.20 g, 0.35 mmol) in MeOH (10 mL), purged with nitrogen, was added Pd(OH)₂ (0.10 g, 0.1424 mmol). The system was evacuated and purged with hydrogen (balloon) for 3 hours. The mixture was filtered through a pad of celite and the filtrate was concentrated in vacuo. The resulting residue was purified by silica gel chromatography using 0-80% EtOAc/heptanes gradient to afford 168 mg of product as mixture of cis (major) and trans (minor) isomers. Ethyl 4-[1-(4-fluoro-3-methyl-phenyl)-5-hydroxy-2-tetrahydropyran-4-yl-indol-3-yl]cyclohexane-carboxylate (100%). ESI-MS m/z calc. 479.25, found 480.56 (M+1)⁺. To a solution of ethyl 4-[1-(4-fluoro-3-methyl-phenyl)-5-hydroxy-2-tetrahydropyran-4-yl-indol-3-yl]cyclohexane-carboxylate (168 mg) in MeOH (5 mL), THE (1 mL) and water (1 mL) was added LiOH (0.10 g, 4.18 mmol). The reaction mixture was stirred at room temperature for 16 h. The solvent was evaporated under reduced pressure. The crude residue was acidified with 10% HCl and extracted twice with EtOAc. The organic phase was dried (MgSO₄), filtered, and concentrated in vacuo.

The resulting residue was purified by silica gel chromatography 0-80% EtOAc/heptanes gradient to afford 110 mg (63%) of major product 1 and 10 mg (6%) of minor product 2. Major product 1 trans-4-[1-(4-fluoro-3-methyl-phenyl)-5-hydroxy-2-tetrahydropyran-4-yl-indol-3-yl] cyclohexanecarboxylic acid. ¹H NMR (400 MHz, Methanol-d4) δ 7.25-7.13 (m, 2H), 7.13-7.02 (m, 2H), 6.59 (d, J=8.7 Hz, 1H), 6.53 (dd, J=8.7, 2.3 Hz, 1H), 3.95 (dd, J=11.6, 4.1 Hz, 2H), 3.30-3.28 (m, 2H), 3.06 (m, 1H), 2.83-2.81 (m, 2H), 2.42-2.27 (m, 7H), 2.18-1.96 (m, 2H), 1.80-1.60 (m, 6H). ESI-MS m/z calc. 451.22, found 452.56 (M+1)⁺. Minor product 2 cis-4-[1-(4-fluoro-3-methyl-phenyl)-5-hydroxy-2-tetrahydropyran-4-yl-indol-3-yl]cyclohexanecarboxylic acid. ¹H NMR (400 MHz, Methanol-d4) δ 7.26-7.14 (m, 2H), 7.10 (dd, J=8.5, 2.6 Hz, 2H), 6.61 (d, J=8.7 Hz, 1H), 6.55 (dd, J=8.7, 2.3 Hz, 1H), 3.96 (dd, J=11.5, 4.1 Hz, 2H), 3.30-3.30 (m, 2H), 3.05 (m, 1H), 2.90-2.74 (m, 1H), 2.55-2.45 (m, 1H), 2.34 (d, J=1.9 Hz, 3H), 2.23-1.98 (m, 7H), 1.88-1.86 (m, 2H), 1.70-1.55 (m, 4H). ESI-MS m/z calc. 451.22, found 452.56 (M+1)⁺.

Compounds 3-104 were prepared as described for compounds 1 and 2 by reductive alkylation with the appropriate aldehyde or ketone reagent, and the relevant indole intermediate.

TABLE 7
Method of preparation, structure and physicochemical data for compounds 3-104

| | | Aldehyde or | |
|---|---|---|---|
| Compound | Method/Product | Ketone | ¹H NMR; LCMS m/z [M + H]⁺ |
| 3 | From S5^{1,2,3} 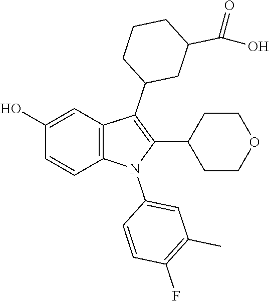 [SMILES: Cc1cc(-n2c(C3CCOCC3)c(C3CCCC(C(=O)O)C3)c3cc(O)ccc32)ccc1F] | 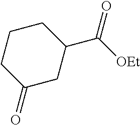 [SMILES: CCOC(=O)C1CCCC(=O)C1] | ¹H NMR (400 MHz, Methanol-d4) δ 7.25-7.16 (m, 2H), 7.12-7.10 (m, 2H), 6.63-6.60 (m, 1H), 6.56-6.53 (m, 1H), 3.97-3.94 (m, 2H), 3.36-3.23 (m, 2H), 3.17- 3.03 (m, 1H), 2.83-2.80 (m, 1H), 2.60-2.49 (m, 1H), 2.34 (d, J = 2.0 Hz, 3H), 2.30- 2.16 (m, 1H), 2.07-2.00 (m, 5H), 1.82-1.80 (m, 2H), 1.74- 1.45 (m, 4H). LCMS m/z 452.56 [M + H]⁺. |
| 4 | From S5^{1,3,4} 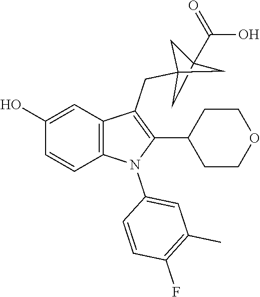 [SMILES: Cc1cc(-n2c(C3CCOCC3)c(CC34CC(C(=O)O)(C3)C4)c3cc(O)ccc32)ccc1F] | 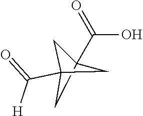 | ¹H NMR (400 MHz, Methanol-d4) δ 7.26-7.18 (m, 2H), 7.15-7.07 (m, 1H), 6.85 (dd, J = 2.0, 1.0 Hz, 1H), 6.58 (dd, J = 2.1, 1.4 Hz, 2H), 3.93 (dd, J = 11.6, 4.0 Hz, 2H), 3.34-3.27 (m, 4H), 3.04 (s, 2H), 2.89 (m, 1H), 2.34 (d, J = 1.9 Hz, 3H), 1.98-1.91 (m, 6H), 1.66-1.62 (m, 2H). LCMS m/z 450.54 [M + H]⁺. |
| 5 | From S2^{1,3,4} 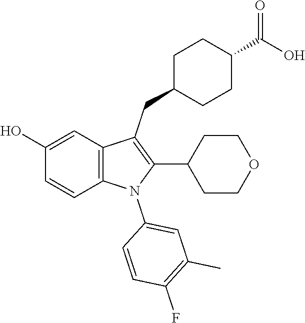 | 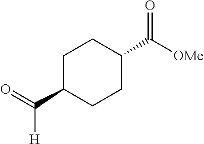 [SMILES: COC(=O)[C@H]1CC[C@H](C=O)CC1] | ¹H NMR (400 MHz, Chloroform-d) δ 7.54-7.49 (m, 2H), 7.44-7.40 (m, 2H), 7.37-7.31 (m, 1H), 7.19- 6.99 (m, 4H), 6.84 (dd, J = 8.8, 2.4 Hz, 1H), 6.74 (dd, J = 8.8, 0.5 Hz, 1H), 5.15 (s, 2H), 3.99 (dd, J = 11.4, 4.1 Hz, 2H), 3.69 (s, 3H), 3.33 (t, J = 11.6 Hz, 2H), 2.97-2.85 (m, 1H), 2.71 (d, J = 7.2 Hz, 2H), 2.36 (d, J = 1.9 Hz, 3H), 2.32- 2.30 (m, 1H), 2.03-1.84 (m, 5H), 1.63-1.60 (m, 3H), 1.40- 1.37 (m,, 2H), 1.16-1.02 (m, 2H). LCMS m/z 570.51 [M + H]⁺. |
| 6 | From S8^{1,3,4,15} 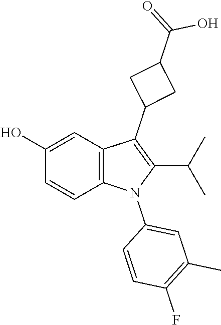 [SMILES: Cc1cc(-n2c(C(C)C)c(C3CC(C(=O)O)C3)c3cc(O)ccc32)ccc1F] | 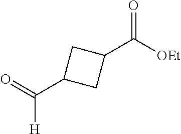 [SMILES: CCOC(=O)C1CC(C=O)C1] | LCMS m/z 382.3 [M + H]⁺. |
| 7 | From S3^{1,4,5,6} 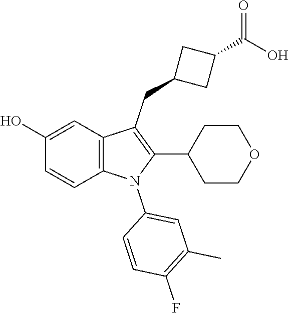 [SMILES: Cc1cc(-n2c(C3CCOCC3)c(C[C@H]3C[C@H](C(=O)O)C3)c3cc(O)ccc32)ccc1F] | 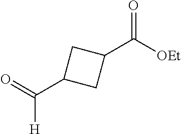 [SMILES: CCOC(=O)C1CC(C=O)C1] | ¹H NMR (400 MHz, Methanol-d4) δ 7.26-7.16 (m, 2H), 7.12 (ddd, J = 9.2, 4.6, 2.7 Hz, 1H), 6.93 (dd, J = 1.9, 1.0 Hz, 1H), 6.61-6.51 (m, 2H), 3.92 (dd, J = 11.5, 4.1 Hz, 2H), 3.34 (dd, J = 11.9, 2.0 Hz, 2H), 3.27-3.11 (m, 1H), 3.03-2.88 (m, 3H), 2.86-2.75 (m, 1H), 2.43- 2.26 (m, 5H), 2.12 (ddd, J = 11.9, 9.1, 6.1 Hz, 2H), 2.06- 1.88 (m, 2H), 1.65 (d, J = 13.3 Hz, 2H). LCMS m/z 438.0 [M + H]⁺ |
| 8 | From S3^{1,4,5,6} 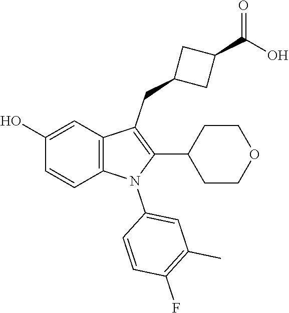 [SMILES: Cc1cc(-n2c(C3CCOCC3)c(C[C@H]3C[C@@H](C(=O)O)C3)c3cc(O)ccc32)ccc1F] | 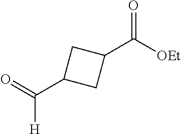 [SMILES: CCOC(=O)C1CC(C=O)C1] | ¹H NMR (400 MHz, Methanol-d4) δ 7.26-7.16 (m, 2H), 7.12 (ddd, J = 9.2, 4.6, 2.7 Hz, 1H), 6.93 (dd, J = 1.9, 1.0 Hz, 1H), 6.61-6.51 (m, 2H), 3.92 (dd, J = 11.5, 4.1 Hz, 2H), 3.34 (dd, J = 11.9, 2.0 Hz, 2H), 3.27-3.11 (m, 1H), 3.03-2.88 (m, 3H), 2.86-2.75 (m, 1H), 2.43- 2.26 (m, 5H), 2.12 (ddd, J = 11.9, 9.1, 6.1 Hz, 2H), 2.06- 1.88 (m, 2H), 1.65 (d, J = 13.3 Hz, 2H). LCMS m/z 438.0 [M + H]⁺ |
| 9 | From S5^{1,2} 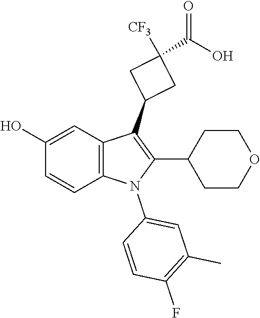 [SMILES: Cc1cc(-n2c(C3CCOCC3)c([C@H]3C[C@@](C(=O)O)(C(F)(F)F)C3)c3cc(O)ccc32)ccc1F] | 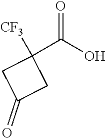 [SMILES: O=C1CC(C(=O)O)(C(F)(F)F)C1] | ¹H NMR (400 MHz, Chloroform-d) δ 7.38 (d, J = 2.4 Hz, 1H), 7.18 (t, J = 8.8 Hz, 1H), 7.08 (ddd, J = 20.5, 7.5, 3.6 Hz, 2H), 6.78 (d, J = 8.8 Hz, 1H), 6.71 (dd, J = 8.7, 2.3 Hz, 1H), 4.20 (dd, J = 11.4, 4.6 Hz, 3H), 3.33 (dt, J = 33.3, 11.6 Hz, 4H), 2.97- 2.72 (m, 3H), 2.37 (d, J = 1.9 Hz, 3H), 2.16 (d, J = 12.9 Hz, 2H), 1.69 (d, J = 13.6 Hz, 2H). LCMS m/z 492.0 [M + H]⁺. |
| 10 | From S5^{1,3} 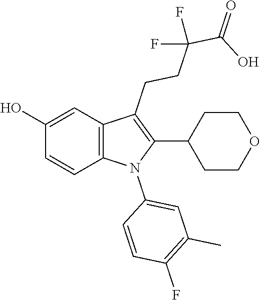 [SMILES: Cc1cc(-n2c(C3CCOCC3)c(CCC(F)(F)C(=O)O)c3cc(O)ccc32)ccc1F] | 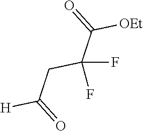 [SMILES: CCOC(=O)C(F)(F)CC=O] | ¹H NMR (400 MHz, Methanol-d4) δ 7.28-7.18 (m, 2H), 7.13-7.11 (m, 1H), 6.87 (dd, J = 2.2, 0.7 Hz, 1H), 6.68-6.51 (m, 2H), 4.01- 3.88 (m, 2H), 3.34-3.31 (m, 2H), 3.32-3.00 (m, 2H), 2.85- 2.82 (m, 1H), 2.47-2.40 (m, 2H), 2.34 (d, J = 2.0 Hz, 3H), 2.08-1.97 (m, 2H), 1.69 (d, J = 12.6 Hz, 3H). |
| 11 | From S5^{1,3,4} 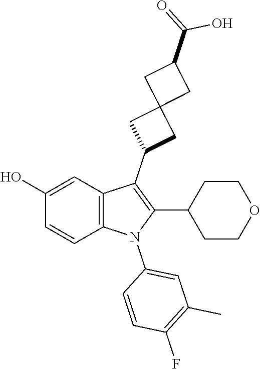 | 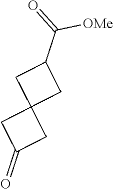 [SMILES: COC(=O)C1CC2(CC(=O)C2)C1] | ¹H NMR (400 MHz, DMSO- d6) δ 12. 07 (s, 1H), 8.77 (s, 1H), 7.37-7.24 (m, 2H), 7.18 (dd, J = 8.2, 4.3 Hz, 1H), 7.10 (d, J = 2.2 Hz, 1H), 6.57 (d, J = 8.7 Hz, 1H), 6.53-6.30 (m, 1H), 3.90-3.60 (m, 3H), 3.18 (t, J = 11.7 Hz, 2H), 3.03- 3.01 (m, 1H), 2.80-2.56 (m, 3H), 2.53-2.42 (m, 3H), 2.38- 2.20 (m, 6H), 1.86-1.83 (m, 2H), 1.58-1.56 (m, 2H). LCMS m/z 464.1 [M + H]⁺ |
| 12 | From S5^{1,3,4} 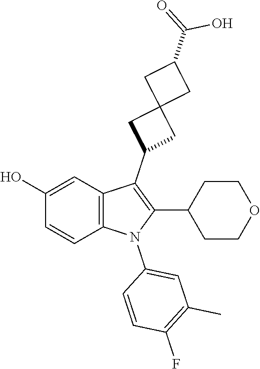 | 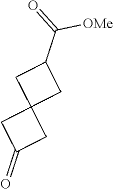 [SMILES: COC(=O)C1CC2(CC(=O)C2)C1] | ¹H NMR (400 MHz, DMSO- d6) δ 8.77 (s, 1H), 7.38-7.24 (m, 2H), 7.17-7.15 (m, 1H), 7.10 (d, J = 2.1 Hz, 1H), 6.57 (d, J = 8.7 Hz, 1H), 6.52 (dd, J = 8.8, 2.1 Hz, 1H), 3.92- 3.70 (m, 3H), 3.17 (t, J = 11.6 Hz, 2H), 3.02 (p, J = 8.4 Hz, 1H), 2.79-2.57 (m, 3H), 2.50-2.43 (m, 2H), 2.40-2.04 (m, 7H), 1.94-1.71 (m, 2H), 1.60-1.57 (m, 2H). LCMS m/z 464.0 [M + H]⁺ |
| 13 | From S5^{1,3,7} 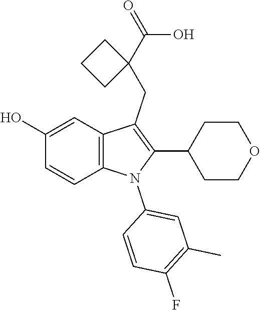 [SMILES: Cc1cc(-n2c(C3CCOCC3)c(CC3(C(=O)O)CCC3)c3cc(O)ccc32)ccc1F] | 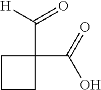 | ¹H NMR (400 MHz, Chloroform-d) δ 7.18-7.08 (m, 3H), 6.99 (d, J = 2.3 Hz, 1H), 6.67-6.50 (m, 2H), 3.92 (d, J = 11.4 Hz, 3H), 3.35 (t, J = 11.7 Hz, 2H), 3.26 (s, 2H), 3.11 (t, J = 12.4 Hz, 1H), 2.47- 2.36 (m, 2H), 2.35-2.28 (m, 3H), 2.22 (dd, J = 13.1, 6.1 Hz, 2H), 1.96-1.72 (m, 5H), 1.57 (d, J = 12.9 Hz, 2H). LCMS m/z 438.35 [M + H]⁺ |
| 14 | From S5^{1,3,4} 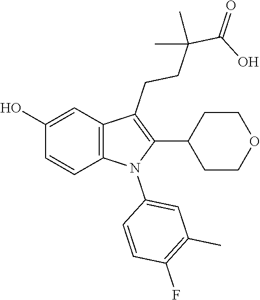 [SMILES: Cc1cc(-n2c(C3CCOCC3)c(CCC(C)(C)C(=O)O)c3cc(O)ccc32)ccc1F] | 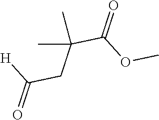 | ¹H NMR (400 MHz, DMSO- d6) δ 12.34 (s, 1H), 8.74 (s, 1H), 7.45-7.24 (m, 2H), 7.23- 7.07 (m, 1H), 6.81 (d, J = 2.2 Hz, 1H), 6.60 (s, 1H), 6.53 (dd, J = 8.7, 2.1 Hz, 1H), 3.92-3.84 (m, 2H), 3.16 (t, J = 11.6 Hz, 2H), 2.85-2.64 (m, 3H), 2.30 (d, J = 1.9 Hz, 3H), 1.90-1.88 (m, 2H), 1.78- 1.75 (m, 2H), 1.63 (m, 2H), 1.24 (s, 6H). LCMS m/z 440.2 [M + H]⁺ |
| 15 | From S5^{1,3,4} 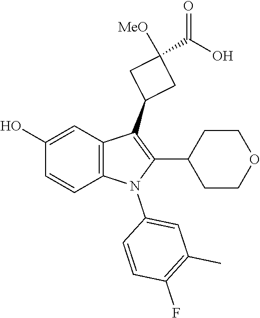 [SMILES: CO[C@]1(C(=O)O)C[C@H](c2c(C3CCOCC3)n(-c3ccc(F)c(C)c3)c3ccc(O)cc32)C1] | 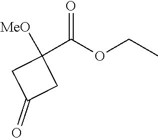 | ¹H NMR (400 MHz, DMSO- d6) δ 12.87 (s, 1H), 8.85 (s, 1H), 7.36-7.23 (m, 3H), 7.22- 7.15 (m, 1H), 6.64-6.53 (m, 2H), 3.91-3.83 (m, 2H), 3.82-3.71 (m, 1H), 3.27 (s, 3H), 3.21-3.11 (m, 2H), 2.75- 2.67 (m, 5H), 2.30 (s, 3H), 1.94-1.79 (m, 2H), 1.67- 1.56 (m, 2H). LCMS m/z 454.23 [M + H]⁺ |
| 16 | From S5^{1,3,4} 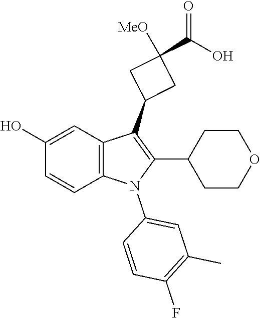 [SMILES: CO[C@]1(C(=O)O)C[C@@H](c2c(C3CCOCC3)n(-c3ccc(F)c(C)c3)c3ccc(O)cc32)C1] | 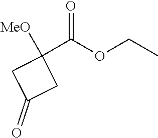 | ¹H NMR (400 MHz, DMSO- d6) δ 12.92 (s, 1H), 8.74 (s, 1H), 7.36-7.27 (m, 3H), 7.22- 7.16 (m, 1H), 6.58 (s, 2H), 4.04 (p, J = 9.8 Hz, 1H), 3.91- 3.83 (m, 2H), 3.31 (s, 3H), 3.23-3.13 (m, 2H), 3.04- 2.94 (m, 2H), 2.81-2.67 (m, 1H), 2.47-2.39 (m, 2H), 2.30 (s, 3H), 1.94-1.78 (m, 2H), 1.67-1.56 (m, 2H). LCMS m/z 453.20 [M + H]⁺ |
| 17 | From S5^{1,2} 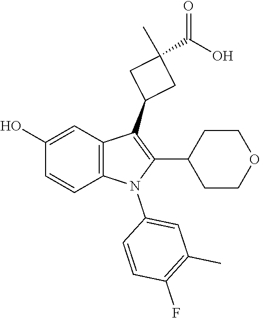 [SMILES: Cc1cc(-n2c(C3CCOCC3)c([C@H]3C[C@](C)(C(=O)O)C3)c3cc(O)ccc32)ccc1F] | 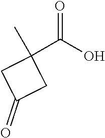 | ¹H NMR (400 MHz, Methanol-d4) δ 7.34 (dd, J = 2.2, 0.7 Hz, 1H), 7.26-7.13 (m, 2H), 7.08 (ddd, J = 8.1, 4.5, 2.7 Hz, 1H), 6.67-6.54 (m, 2H), 4.13-4.01 (m, 1H), 3.93 (dd, J = 11.6, 4.2 Hz, 2H), 3.26 (dd, J = 11.9, 2.0 Hz, 2H), 2.88-2.57 (m, 5H), 2.33 (d, J = 1.9 Hz, 3H), 2.03 (tdd, J = 12.6, 8.7, 3.8 Hz, 2H), 1.61 (s, 5H). LCMS m/z 437.20 [M + H]⁺ |
| 18 | From S5^{1,2} 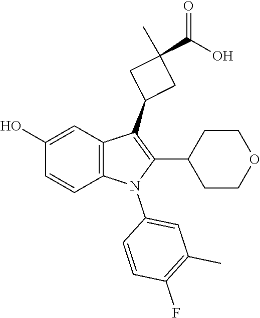 [SMILES: Cc1cc(-n2c(C3CCOCC3)c([C@H]3C[C@@](C)(C(=O)O)C3)c3cc(O)ccc32)ccc1F] | 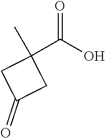 | ¹H NMR (400 MHz, Methanol-d4) δ 7.43 (d, J = 2.1 Hz, 1H), 7.28-7.00 (m, 4H), 6.70-6.52 (m, 3H), 4.13- 3.76 (m, 4H), 3.16 (t, J = 10.8 Hz, 2H), 2.98-2.59 (m, 3H), 2.33 (s, 3H), 2.27-1.92 (m, 4H), 1.75 (td, J = 9.7, 8.6, 3.6 Hz, 1H), 1.63 (d, J = 17.5 Hz, 5H). LCMS m/z 438.75 [M + H]⁺ |
| 19 | From S5^{1,3,7} 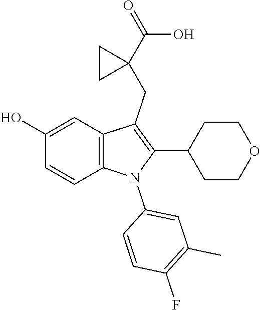 [SMILES: Cc1cc(-n2c(C3CCOCC3)c(CC3(C(=O)O)CC3)c3cc(O)ccc32)ccc1F] | 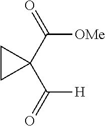 | ¹H NMR (400 MHz, Methanol-d4) δ 7.25-7.15 (m, 2H), 7.13 (ddd, J = 8.9, 4.6, 2.7 Hz, 1H), 6.89 (dd, J = 1.9, 1.0 Hz, 1H), 6.61-6.47 (m, 2H), 3.93-3.81 (m, 2H), 3.47 (s, 2H), 3.39-3.31 (m, 5H), 3.10-2.96 (m, 1H), 2.34 (d, J = 1.9 Hz, 3H), 2.04- 1.82 (m, 2H), 1.62 (d, J = 13.5 Hz, 3H), 1.28 (s, 2H), 0.92 (q, J = 3.3 Hz, 2H), 0.45 (q, J = 3.4 Hz, 2H). LCMS m/z 424.37 [M + H]⁺ |
| 20 | From S5^{1,5,2} 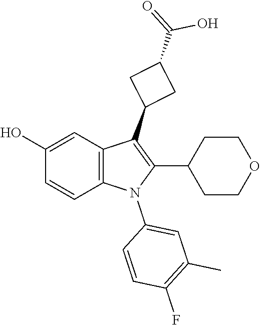 [SMILES: Cc1cc(-n2c(C3CCOCC3)c([C@H]3C[C@H](C(=O)O)C3)c3cc(O)ccc32)ccc1F] | 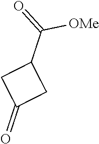 | ¹H NMR (400 MHz, Methanol-d4) δ 7.32 (d, J = 2.2 Hz, 1H), 7.28-7.13 (m, 2H), 7.14-7.01 (m, 1H), 6.67- 6.54 (m, 2H), 4.27 (t, J = 9.3 Hz, 1H), 3.94 (dd, J = 11.4, 4.3 Hz, 2H), 3.01 (d, J = 10.6 Hz, 2H), 2.80 (s, 0H), 2.69- 2.53 (m, 1H), 2.34 (d, J = 2.0 Hz, 3H), 2.02 (d, J = 11.1 Hz, 1H), 1.62 (d, J = 13.4 Hz, 2H). LCMS m/z 424.7 [M + H]⁺ |
| 21 | From S5^{1,5,2} 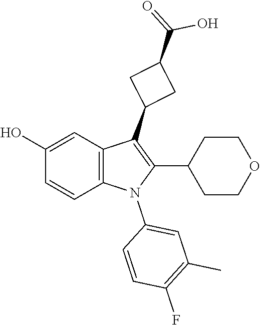 [SMILES: Cc1cc(-n2c(C3CCOCC3)c([C@H]3C[C@@H](C(=O)O)C3)c3cc(O)ccc32)ccc1F] | 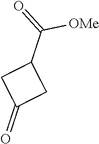 | ¹H NMR (400 MHz, Methanol-d4) δ 7.50 (d, J = 2.1 Hz, 1H), 7.29-7.17 (m, 2H), 7.10 (dt, J = 7.9, 3.5 Hz, 1H), 6.70-6.51 (m, 2H), 3.97 (h, J = 6.4 Hz, 3H), 3.23 (p, J = 9.2 Hz, 1H), 3.00 (q, J = 10.4 Hz, 2H), 2.86 (tt, J = 12.5, 3.9 Hz, 1H), 2.55 (qd, J = 8.5, 2.6 Hz, 2H), 2.36 (d, J = 2.0 Hz, 3H), 2.17-1.97 (m, 3H), 1.66 (d, J = 13.4 Hz, 2H). LCMS m/z 424.0 [M + H]⁺ |
| 22 | From S18^{1,8} 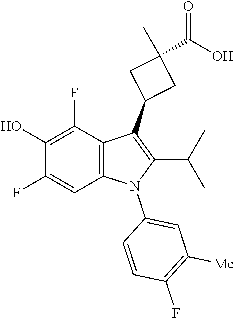 [SMILES: Cc1cc(-n2c(C(C)C)c([C@H]3C[C@](C)(C(=O)O)C3)c3c(F)c(O)c(F)cc32)ccc1F] | 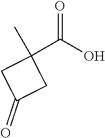 | ¹H NMR (400 MHz, Chloroform-d) δ 7.17 (t, J = 8.7 Hz, 1H), 7.13-7.02 (m, 2H), 6.42 (dd, J = 10.2, 1.4 Hz, 1H), 4.30-4.12 (m, 1H), 2.97 (h, J = 7.2 Hz, 1H), 2.88- 2.79 (m, 2H), 2.70-2.57 (m, 2H), 2.36 (d, J = 1.9 Hz, 3H), 1.30 (dd, J = 7.2, 2.4 Hz, 6H). LCMS m/z 432.5 [M + H]⁺ |
| 23 | From S18^{1,8} 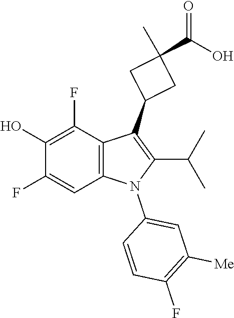 [SMILES: Cc1cc(-n2c(C(C)C)c([C@H]3C[C@@](C)(C(=O)O)C3)c3c(F)c(O)c(F)cc32)ccc1F] | 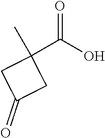 | ¹H NMR (400 MHz, Chloroform-d) δ 7.07 (t, J = 8.8 Hz, 1H), 6.98 (ddt, J = 15.7, 7.8, 2.8 Hz, 2H), 6.30 (dd, J = 10.3, 1.5 Hz, 1H), 3.96-3.80 (m, 1H), 3.00- 2.78 (m, 3H), 2.33-2.13 (m, 2H), 1.98 (s, 3H), 1.61 (s, 3H), 1.27-1.15 (m, 6H). LCMS m/z 432.5 [M + H]⁺ |
| 24 | From S3^{1,2,3} 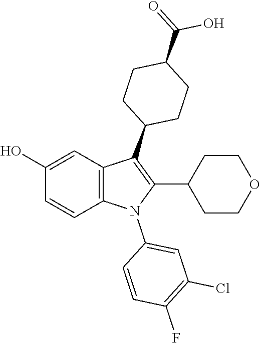 [SMILES: O=C(O)[C@H]1CC[C@@H](c2c(C3CCOCC3)n(-c3ccc(F)c(Cl)c3)c3ccc(O)cc32)CC1] | 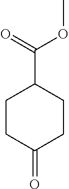 | ¹H NMR (400 MHz, DMSO- d6) δ 12.25 (s, 1H), 8.72 (s, 1H), 7.72 (dd, J = 6.7, 2.6 Hz, 1H), 7.60 (t, J = 8.9 Hz, 1H), 7.38-7.36 (m, 1H), 6.91 (d, J = 2.2 Hz, 1H), 6.63 (s, 1H), 6.55 (dd, J = 8.8, 2.2 Hz, 1H), 3.87-3.84 (m, 2H), 3.23-3.02 (m, 3H), 2.99 (m, 1H), 2.80- 2.66 (m, 2H), 2.18-2.15 (m, 3H), 1.98-1.82 (m, 2H), 1.72- 1.48 (m, 6H). LCMS m/z 472.59 [M + H]⁺ |
| 25 | From S5^{1,2,5,6,15} 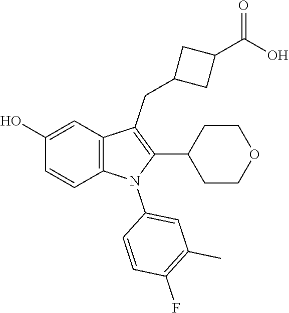 [SMILES: Cc1cc(-n2c(C3CCOCC3)c(CC3CC(C(=O)O)C3)c3cc(O)ccc32)ccc1F] | 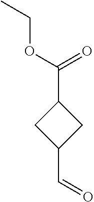 | LCMS m/z 438.5 [M + H]⁺ |
| 26 | From S8^{1,3,7,6,15} 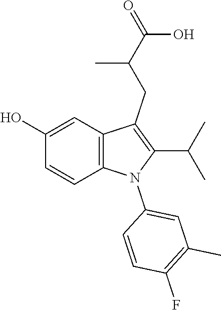 [SMILES: Cc1cc(-n2c(C(C)C)c(CC(C)C(=O)O)c3cc(O)ccc32)ccc1F] | 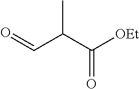 | ¹H NMR (400 MHz, Chloroform-d) δ 7.20-7.06 (m, 3H), 7.00 (dd, J = 2.1, 0.9 Hz, 1H), 6.77-6.54 (m, 2H), 3.33-3.18 (m, 1H), 3.11 (hept, J = 7.2 Hz, 1H), 2.99- 2.78 (m, 2H), 2.33 (dd, J = 2.1, 1.1 Hz, 3H), 1.31-1.10 (m, 9H). LCMS m/z 369.4 [M + H]⁺ |
| 27 | From S3^{8,2} 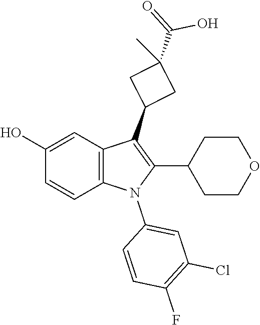 [SMILES: C[C@]1(C(=O)O)C[C@H](c2c(C3CCOCC3)n(-c3ccc(F)c(Cl)c3)c3ccc(O)cc32)C1] | 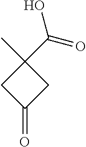 | ¹H NMR (300 MHz, Methanol-d4) δ 7.55-7.42 (m, 2H), 7.35 (dd, J = 2.1, 0.8 Hz, 1H), 7.31-7.24 (m, 1H), 6.71-6.52 (m, 2H), 4.07 (t, J = 9.6 Hz, 1H), 3.95 (dd, J = 11.6, 4.2 Hz, 2H), 2.88-2.62 (m, 4H), 2.20-1.94 (m, 1H), 1.65-1.55 (m, 4H). LCMS m/z 457.0 [M + H]⁺ |
| 28 | From S3^{8,2} 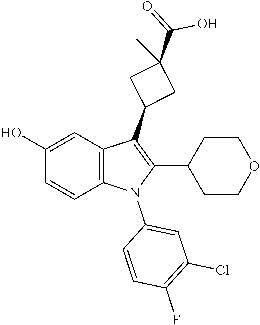 [SMILES: C[C@]1(C(=O)O)C[C@@H](c2c(C3CCOCC3)n(-c3ccc(F)c(Cl)c3)c3ccc(O)cc32)C1] | 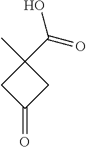 | ¹H NMR (300 MHz, Methanol-d4) δ 7.57-7.39 (m, 3H), 7.25 (ddd, J = 8.7, 4.3, 2.5 Hz, 1H), 6.72-6.56 (m, 2H), 4.16-3.90 (m, 3H), 3.37 (d, J = 2.1 Hz, 1H), 3.16 (td, J = 10.0, 2.6 Hz, 2H), 2.83 (tt, J = 12.4, 3.8 Hz, 1H), 2.28-1.85 (m, 4H), 1.65-1.58 (m, 5H). LCMS m/z 458.1 [M + H]⁺ |
| 29 | From S3^{1,10,4} 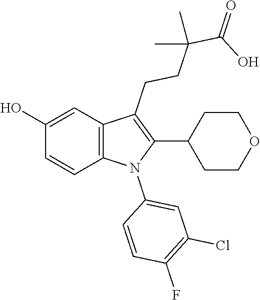 [SMILES: CC(C)(CCc1c(C2CCOCC2)n(-c2ccc(F)c(Cl)c2)c2ccc(O)cc12)C(=O)O] | 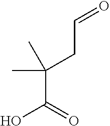 | ¹H NMR (400 MHz, DMSO- d6) δ 12.35 (s, 1H), 8.80 (s, 1H), 7.71 (d, J = 2.4 Hz, 1H), 7.61 (t, J = 8.9 Hz, 1H), 7.40- 7.37 (m, 1H), 6.82 (d, J = 2.1 Hz, 1H), 6.64 (d, J = 8.6 Hz, 1H), 6.57-6.41 (m, 1H), 3.90- 3.70 (m, 2H), 3.19 (t, J = 11.6 Hz, 2H), 2.81-2.61 (m, 3H), 1.89-1.84 (m, 2H), 1.79- 1.72 (m, 2H), 1.65-1.62 (m, 2H), 1.24 (s, 6H). LCMS m/z 460.1 [M + H]⁺ |
| 30 | From S1^{1,10,4,11} 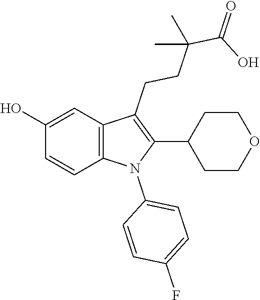 [SMILES: CC(C)(CCc1c(C2CCOCC2)n(-c2ccc(F)cc2)c2ccc(O)cc12)C(=O)O] | 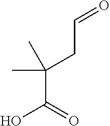 | ¹H NMR (400 MHz, DMSO- d6) δ 12.34 (s, 1H), 8.76 (s, 1H), 7.40 (d, J = 7.8 Hz, 4H), 6.56 (q, J = 8.7 Hz, 2H), 3.97- 3.71 (m, 2H), 3.16 (t, J = 11.7 Hz, 2H), 2.72-2.68 (m, 3H), 1.96-1.83 (m, 2H), 1.78-1.75 (m,1H), 1.62 (d, J = 14.3 Hz, 2H), 1.24 (s, 6H). LCMS m/z 426.2 [M + H]⁺ |
| 31 | From S3^{1,3,4} 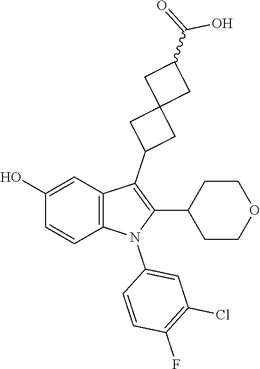 [SMILES: O=C(O)C1CC2(C1)CC(c1c(C3CCOCC3)n(-c3ccc(F)c(Cl)c3)c3ccc(O)cc13)C2] | 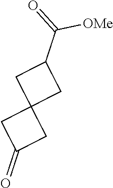 [SMILES: COC(=O)C1CC2(CC(=O)C2)C1] | ¹H NMR (400 MHz, Chloroform-d) δ 7.38-7.34 (m, 1H), 7.31 (d, J = 8.6 Hz, 1H), 7.26 (d, J = 2.3 Hz, 1H), 7.20-6.92 (m, 1H), 6.75 (dd, J = 8.7, 0.5 Hz, 1H), 6.68 (dd, J = 8.7, 2.3 Hz, 1H), 4.05 (dd, J = 11.6, 4.2 Hz, 2H), 3.87 (p, J = 9.3 Hz, 1H), 3.37-3.31 (m,, 2H), 3.22-3.18 (m, 1H), 2.84-2.74 (m, 3H), 2.62-2.31 (m, 6H), 2.09-2.05 (m, 2H), 1.64-01.61 (m, 2H). LCMS m/z 484.3 [M + H]⁺ |
| 32 | From S3^{1,3,4,6} 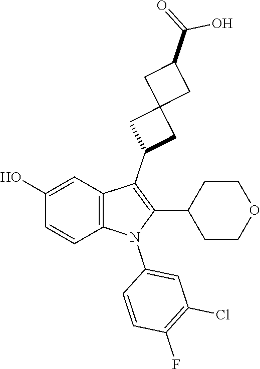 | 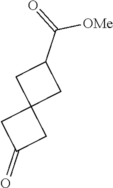 [SMILES: COC(=O)C1CC2(CC(=O)C2)C1] | ¹H NMR (400 MHz, Methanol-d4) δ 7.50-7.36 (m, 2H), 7.29-7.20 (m, 2H), 6.70-6.56 (m, 2H), 4.02- 3.91 (m, 2H), 3.86-3.83 (m, 1H), 3.32-3.31 (m, 3H), 3.11- 3.07 (m, 1H), 2.82-2.74 (m, 3H), 2.58-2.23 (m, 5H), 2.03-1.99 (m, 2H), 1.66-1.63 (m, 2H). LCMS m/z 484.2 [M + H]⁺ |
| 33 | From S3^{1,3,4,6} 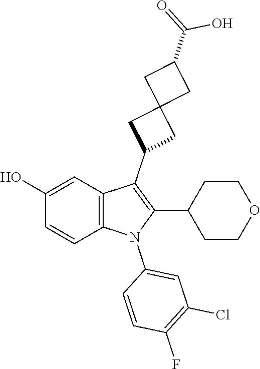 | 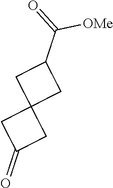 [SMILES: COC(=O)C1CC2(CC(=O)C2)C1] | ¹H NMR (400 MHz, Chloroform-d) δ 7.40-7.23 (m, 3H), 7.18 (m, 1H), 6.75 (d, J = 8.7 Hz, 1H), 6.68 (dd, J = 8.7, 2.3 Hz, 1H), 4.05 (d, J = 11.0 Hz, 2H), 3.89-3.84 (m, 1H), 3.37-3.34 (m, 2H), 3.20-3.17 (m, J = 8.5 Hz, 1H), 2.84-2.78 (m, 3H), 2.59- 2.49 (m, 6H), 2.07-2.04 (m, 2H), 1.63 (d, J = 13.3 Hz, 2H). LCMS m/z 484.3 [M + H]⁺ |
| 34 | From S3^{1,4,5} 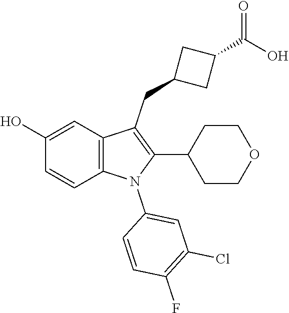 [SMILES: O=C(O)[C@H]1C[C@H](Cc2c(C3CCOCC3)n(-c3ccc(F)c(Cl)c3)c3ccc(O)cc23)C1] | 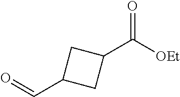 [SMILES: CCOC(=O)C1CC(C=O)C1] | ¹H NMR (400 MHz, Methanol-d4) δ 7.49 (dd, J = 6.6, 2.6 Hz, 1H), 7.43 (t, J = 8.8 Hz, 1H), 7.28 (ddd, J = 8.7, 4.3, 2.5 Hz, 1H), 6.94 (dd, J = 2.0, 1.0 Hz, 1H), 6.59 (t, J = 1.4 Hz, 2H), 4.02- 3.84 (m, 2H), 3.36 (d, J = 12.3 Hz, 2H), 3.17 (tt, J = 9.5, 5.6 Hz, 1H), 3.04-2.91 (m, 3H), 2.81 (p, J = 7.5 Hz, 1H), 2.33 (ddd, J = 10.6, 8.1, 5.4 Hz, 2H), 2.12 (ddd, J = 11.6, 9.0, 6.1 Hz, 2H), 2.04-1.86 (m, 2H), 1.67 (d, J = 13.3 Hz, 2H). LCMS m/z 458.5 [M + H]⁺ |
| 35 | From S1^{1,4,5,11} 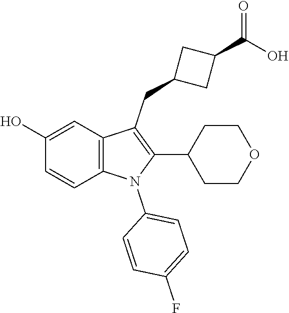 [SMILES: O=C(O)[C@H]1C[C@@H](Cc2c(C3CCOCC3)n(-c3ccc(F)cc3)c3ccc(O)cc23)C1] | 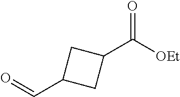 [SMILES: CCOC(=O)C1CC(C=O)C1] | ¹H NMR (400 MHz, Methanol-d4) δ 7.31 (dt, J = 7.9, 2.4 Hz, 4H), 6.99-6.92 (m, 1H), 6.63-6.51 (m, 2H), 3.93 (dd, J = 11.5, 4.1 Hz, 2H), 3.34 (ddq, J = 7.1, 3.8, 2.0 Hz, 5H), 3.08-2.89 (m, 4H), 2.72-2.56 (m, 1H), 2.39- 2.26 (m, 2H), 2.11 (td, J = 9.3, 2.6 Hz, 2H), 2.04-1.89 (m, 2H), 1.74-1.60 (m, 2H). LCMS m/z 424.4 [M + H]⁺ |
| 36 | From S1^{1,4,5,11} 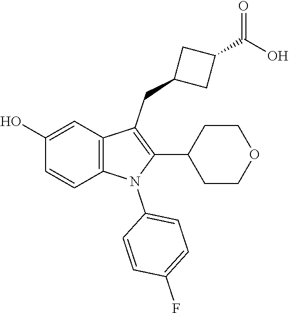 [SMILES: O=C(O)[C@H]1C[C@H](Cc2c(C3CCOCC3)n(-c3ccc(F)cc3)c3ccc(O)cc23)C1] | 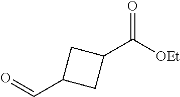 [SMILES: CCOC(=O)C1CC(C=O)C1] | ¹H NMR (400 MHz, Methanol-d4) δ 7.41-7.27 (m, 4H), 6.97 (dd, J = 2.0, 0.9 Hz, 1H), 6.63-6.50 (m, 2H), 3.94 (dd, J = 11.5, 4.2 Hz, 2H), 3.25-3.11 (m, 1H), 2.99 (d, J = 7.9 Hz, 3H), 2.90- 2.76 (m, 1H), 2.44-2.30 (m, 2H), 2.22-2.07 (m, 2H), 2.07- 1.91 (m, 3H), 1.68 (d, J = 13.2 Hz, 2H). LCMS m/z 424.3 [M + H]⁺ |
| 37 | From S3^{1,4} 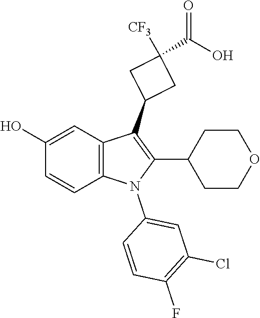 [SMILES: O=C(O)[C@]1(C(F)(F)F)C[C@@H](c2c(C3CCOCC3)n(-c3ccc(F)c(Cl)c3)c3ccc(O)cc32)C1] | 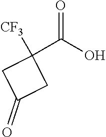 [SMILES: O=C1CC(C(=O)O)(C(F)(F)F)C1] | ¹H NMR (400 MHz, Methanol-d4) δ 7.54-7.45 (m, 2H), 7.33 (dd, J = 1.9, 1.0 Hz, 1H), 7.30 (ddd, J = 8.7, 4.3, 2.5 Hz, 1H), 6.70-6.63 (m, 2H), 4.19 (p, J = 9.7 Hz, 1H), 3.97 (dd, J = 11.6, 4.1 Hz, 2H), 3.24-3.09 (m, 2H), 2.93-2.71 (m, 3H), 2.02 (d, J = 12.9 Hz, 2H), 1.69 (d, J = 13.3 Hz, 2H). LCMS m/z 512.0 [M + H]⁺ |
| 38 | From S3^{1,4} 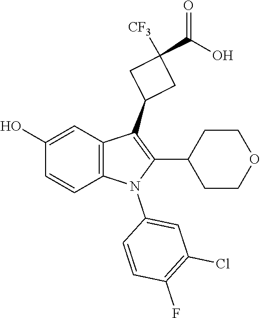 [SMILES: O=C(O)[C@]1(C(F)(F)F)C[C@H](c2c(C3CCOCC3)n(-c3ccc(F)c(Cl)c3)c3ccc(O)cc32)C1] | 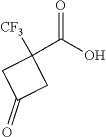 [SMILES: O=C1CC(C(=O)O)(C(F)(F)F)C1] | ¹H NMR (400 MHz, Methanol-d4) δ 7.56 (ddd, J = 6.6, 2.6, 1.5 Hz, 1H), 7.48 (t, J = 8.7 Hz, 1H), 7.42-7.31 (m, 1H), 6.72-6.57 (m, 2H), 6.47 (d, J = 0.7 Hz, 1H), 4.32- 4.10 (m, 1H), 4.02-3.91 (m, 2H), 3.47-3.36 (m, 2H), 3.26-3.10 (m, 1H), 2.98- 2.76 (m, 3H), 1.91-1.71 (m, 4H). LCMS m/z 512.0 [M + H]⁺ |
| 39 | From S4^{1,2} 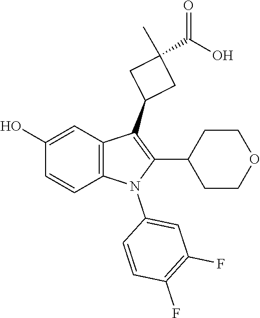 [SMILES: C[C@]1(C(=O)O)C[C@H](c2c(C3CCOCC3)n(-c3ccc(F)c(F)c3)c3ccc(O)cc32)C1] | 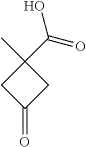 | ¹H NMR (400 MHz, Chloroform-d) δ 7.44-7.31 (m, 2H), 7.15 (ddd, J = 10.1, 7.0, 2.5 Hz, 1H), 7.09-7.02 (m, 1H), 6.79-6.64 (m, 2H), 4.22-3.99 (m, 3H), 3.34 (dt, J = 11.4, 7.0 Hz, 2H), 2.94 (td, J = 9.3, 2.7 Hz, 2H), 2.85- 2.67 (m, 3H), 2.05 (dd, J = 14.7, 10.6 Hz, 2H), 1.70 (s, 3H), 1.63 (d, J = 12.6 Hz, 2H). LCMS m/z 442.5 [M + H]⁺ |
| 40 | From S4^{1,2} 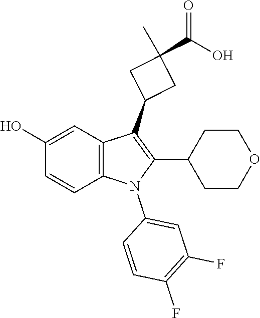 | 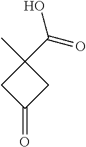 | ¹H NMR (400 MHz, Chloroform-d) δ 7.70 (d, J = 1.7 Hz, 1H), 7.43-7.31 (m, 1H), 7.26-6.97 (m, 2H), 6.77 (d, J = 1.3 Hz, 2H), 6.73- 6.54 (m, 1H), 4.25-3.90 (m, 4H), 3.46-3.24 (m, 5H), 3.06 (td, J = 9.1, 2.7 Hz, 1H), 2.88- 2.73 (m, 2H), 2.26 (dd, J = 11.9, 8.6 Hz, 2H), 2.17-1.96 (m, 2H), 1.98-1.82 (m, 1H), 1.70 (d, J = 14.6 Hz, 4H). LCMS m/z 457.0 [M + H]⁺ |
| 41 | From S4^{1,3,4,6} 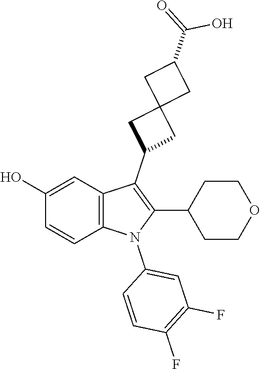 | 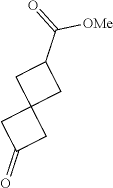 [SMILES: COC(=O)C1CC2(CC(=O)C2)C1] | ¹H NMR (400 MHz, Methanol-d4) δ 7.52-7.43 (m, 1H), 7.34-7.20 (m, 2H), 7.15-6.97 (m, 1H), 6.65 (d, J = 8.7 Hz, 1H), 6.59 (dd, J = 8.7, 2.3 Hz, 1H), 4.00-3.71 (m, 3H), 3.34-3.10 (m, 2H), 3.10-3.07 (m, 1H), 2.86-2.67 (m, 3H), 2.58-2.19 (m, 6H), 2.09-1.91 (m, 2H), 1.70- 1.56 (m, 2H) LCMS m/z 468.2 [M + H]⁺ |
| 42 | From S4^{1,3,4,6} 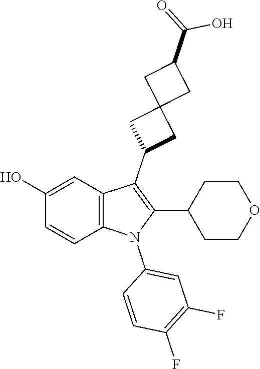 | 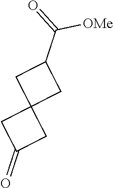 [SMILES: COC(=O)C1CC2(CC(=O)C2)C1] | ¹H NMR (400 MHz, Methanol-d4) δ 7.45 (dt, J = 10.5, 8.8 Hz, 1H), 7.34-7.14 (m, 2H), 7.12-7.09 (m, 1H), 4.03-3.76 (m, 3H), 3.31-3.28 (m, 2H), 3.15-2.95 (m, 2H), 2.76-2.73 (m, 3H), 2.57-2.22 (m, 5H), 2.12-1.84 (m, 2H), 1.66-1.62 (m, 2H). LCMS m/z 468.5 [M + H]⁺ |
| 43 | From S22¹ 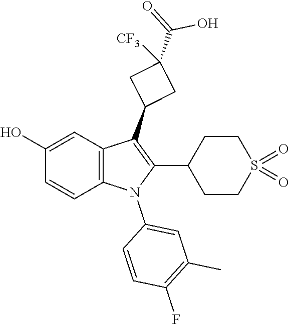 [SMILES: Cc1cc(-n2c(C3CCS(=O)(=O)CC3)c([C@H]3C[C@@](C(=O)O)(C(F)(F)F)C3)c3cc(O)ccc32)ccc1F] | 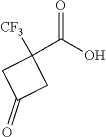 [SMILES: O=C1CC(C(=O)O)(C(F)(F)F)C1] | ¹H NMR (400 MHz, Chloroform-d) δ 7.20 (td, J = 8.7, 3.7 Hz, 1H), 7.16-7.00 (m, 2H), 6.81-6.66 (m, 2H), 6.64-6.50 (m, 1H), 4.59 (p, J = 7.5 Hz, 1H), 4.24 (dd, J = 20.1, 10.1 Hz, 1H), 3.53- 3.38 (m, 1H), 3.31 (td, J = 10.5, 2.8 Hz, 1H), 3.21-3.02 (m, 2H), 3.00-2.80 (m, 4H), 2.54-2.48 (m, 1H), 2.46- 2.34 (m, 3H), 2.28 (d, J = 15.3 Hz, 1H). LCMS m/z 468.5 [M + H]⁺ 540.21 (M + 1)⁺ |
| 44 | From S22^{1,3,4} 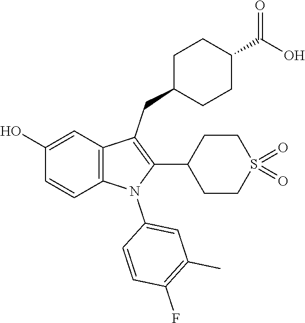 [SMILES: Cc1cc(-n2c(C3CCS(=O)(=O)CC3)c(C[C@H]3CC[C@H](C(=O)O)CC3)c3cc(O)ccc32)ccc1F] | 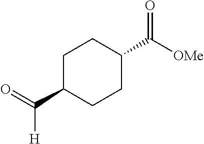 [SMILES: COC(=O)[C@H]1CC[C@H](C=O)CC1] | ¹H NMR (400 MHz, Chloroform-d) δ 7.19 (t, J = 8.7 Hz, 1H), 7.13 (dd, J = 6.7, 2.5 Hz, 1H), 7.08 (dt, J = 7.8, 3.4 Hz, 1H), 7.01-6.94 (m, 1H), 6.75-6.66 (m, 2H), 3.09 (d, J = 13.7 Hz, 2H), 2.90 (dt, J = 25.0, 13.8 Hz, 3H), 2.71 (d, J = 7.1 Hz, 1H), 2.55 (p, J = 12.7 Hz, 2H), 2.41-2.25 (m, 5H), 2.11 (t, J = 14.9 Hz, 5H), 1.93 (d, J = 12.2 Hz, 3H), 1.53-1.36 (m, 4H). LCMS m/z 468.5 [M + H]⁺ 514.2 (M + 1)⁺ |
| 45 | From S1^{1,2,11,6} 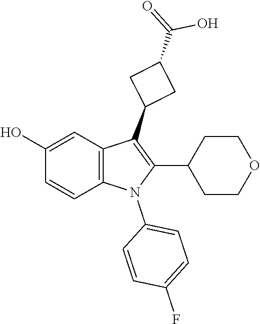 [SMILES: O=C(O)[C@H]1C[C@H](c2c(C3CCOCC3)n(-c3ccc(F)cc3)c3ccc(O)cc32)C1] | 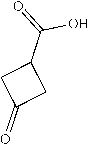 | ¹H NMR (400 MHz, Methanol-d4) δ 7.42-7.21 (m, 6H), 6.67-6.56 (m, 2H), 4.25 (p, J = 9.3 Hz, 1H), 3.92 (dd, J = 11.4, 4.3 Hz, 2H), 3.41-3.20 (m, 2H), 2.98 (qd, J = 9.7, 2.4 Hz, 2H), 2.79 (tt, J = 12.5, 3.7 Hz, 1H), 2.63 (tt, J = 9.7, 3.4 Hz, 2H), 2.10- 1.91 (m, 2H), 1.69-1.52 (m, 2H). LCMS m/z 468.5 [M + H]⁺ 410.5 (M + 1)⁺ |
| 46 | From S1^{1,2,11,6} 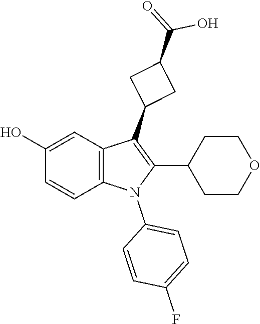 [SMILES: O=C(O)[C@H]1C[C@@H](c2c(C3CCOCC3)n(-c3ccc(F)cc3)c3ccc(O)cc32)C1] | 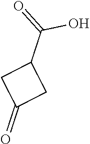 | ¹H NMR (400 MHz, Methanol-d4) δ 7.55 (d, J = 2.2 Hz, 1H), 7.28 (d, J = 6.7 Hz, 4H), 6.69-6.52 (m, 2H), 4.03-3.84 (m, 3H), 3.40- 3.23 (m, 5H), 3.14 (p, J = 9.0 Hz, 1H), 2.97 (qd, J = 9.9, 2.5 Hz, 2H), 2.82 (tt, J = 12.5, 3.8 Hz, 1H), 2.52 (qd, J = 8.6, 2.5 Hz, 2H), 2.04 (qd, J = 12.7, 4.5 Hz, 2H), 1.70-1.56 (m, 2H). LCMS m/z 468.5 [M + H]⁺ 410.5 (M + 1)⁺ |
| 47 | From S22^{1,3,4} 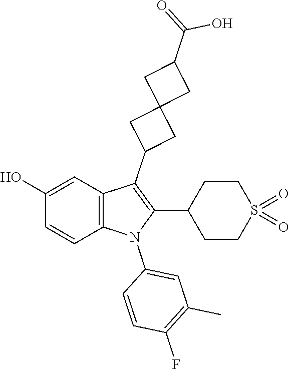 [SMILES: Cc1cc(-n2c(C3CCS(=O)(=O)CC3)c(C3CC4(CC(C(=O)O)C4)C3)c3cc(O)ccc32)ccc1F] | 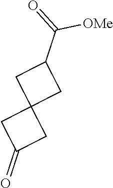 [SMILES: COC(=O)C1CC2(CC(=O)C2)C1] | ¹H NMR (400 MHz, Chloroform-d) δ 7.26 (d, J = 2.3 Hz, 1H), 7.18 (t, J = 8.7 Hz, 1H), 7.09 (d, J = 6.7 Hz, 1H), 7.04 (dd, J = 10.4, 2.2 Hz, 1H), 6.78-6.64 (m, 2H), 3.78 (p, J = 9.3 Hz, 1H), 3.19 (q, J = 8.4 Hz, 1H), 3.09 (d, J = 13.8 Hz, 2H), 2.92 (td, J = 13.7, 3.4 Hz, 2H), 2.78 (q, J = 10.6 Hz, 3H), 2.68-2.50 (m, 5H), 2.50-2.41 (m, 2H), 2.37 (s, 3H), 2.07 (s, 3H). LCMS m/z 512.3 [M + H]⁺ |
| 48 | From S1^{1,2} 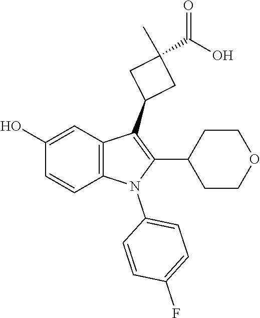 [SMILES: C[C@]1(C(=O)O)C[C@H](c2c(C3CCOCC3)n(-c3ccc(F)cc3)c3ccc(O)cc32)C1] | 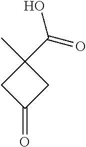 | ¹H NMR (400 MHz, Methanol-d4) δ 7.40-7.24 (m, 5H), 6.68-6.53 (m, 2H), 4.16-4.00 (m, 1H), 3.93 (dd, J = 11.4, 4.3 Hz, 2H), 3.26 (dd, J = 11.9, 2.0 Hz, 2H), 2.89-2.56 (m, 5H), 2.11- 1.94 (m, 3H), 1.67-1.56 (m, 5H). LCMS m/z 424.5 [M + H]⁺ |
| 49 | From S1^{1,2} 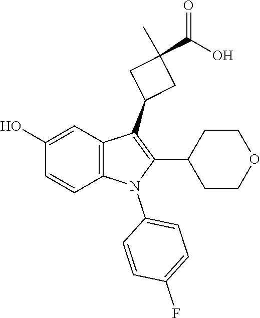 [SMILES: C[C@]1(C(=O)O)C[C@@H](c2c(C3CCOCC3)n(-c3ccc(F)cc3)c3ccc(O)cc32)C1] | 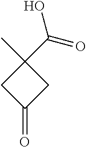 | ¹H NMR (400 MHz, Methanol-d4) δ 7.44 (dd, J = 2.2, 0.8 Hz, 1H), 7.29 (d, J = 6.7 Hz, 4H), 6.67-6.50 (m, 2H), 4.18-3.89 (m, 3H), 3.23- 3.12 (m, 2H), 2.91-2.81 (m, 1H), 2.17 (td, J = 9.0, 2.6 Hz, 2H), 2.03 (dd, J = 13.2, 4.5 Hz, 2H), 1.71-1.49 (m, 5H). LCMS m/z 424.5 [M + H]⁺ |
| 50 | From S1^{1,3,4,6} 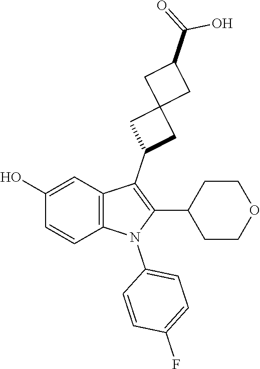 | 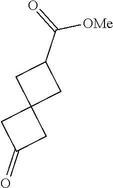 [SMILES: COC(=O)C1CC2(CC(=O)C2)C1] | ¹H NMR (400 MHz, DMSO- d6) δ 7.45-7.28 (m, 4H), 6.62-6.46 (m, 2H), 3.94- 3.73 (m, 3H), 3.21-3.11 (m, 2H), 3.03-3.01 (m, 1H), 2.77- 2.55 (m, 3H), 2.46-2.43 (m, 2H), 2.39-2.14 (m, 4H), 1.84-1.81 (m,, 2H), 1.59-1.56 (m, 2H). LCMS m/z 450.5 [M + H]⁺ |
| 51 | From S1^{1,3,4,6} 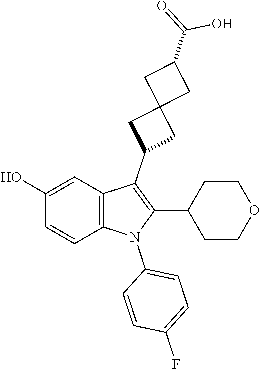 | 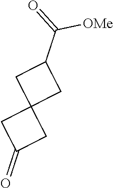 [SMILES: COC(=O)C1CC2(CC(=O)C2)C1] | LCMS m/z 450.5 [M + H]⁺ |
| 52 | From S6^{1,4} 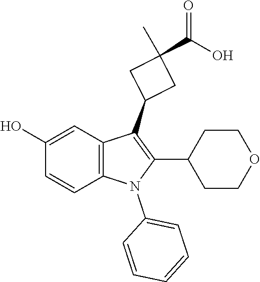 [SMILES: C[C@]1(C(=O)O)C[C@@H](c2c(C3CCOCC3)n(-c3ccccc3)c3ccc(O)cc32)C1] | 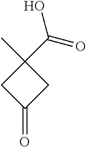 | ¹H NMR (400 MHz, DMSO- d6) δ 12.31 (s, 1H), 8.86 (s, 1H), 7.64-7.45 (m, 3H), 7.33 (d, J = 1.7 Hz, 1H), 7.26 (d, J = 2.2 Hz, 1H), 6.61 (s, 1H), 6.53 (dd, J = 8.7, 2.2 Hz, 1H), 3.98-3.94 (m, 1H), 3.86 (dd, J = 11.5, 3.9 Hz, 2H), 3.19- 2.95 (m, 2H), 2.81-2.67 (m, 2H), 2.54-2.50 (m,3H), 2.02- 1.81 (m, 2H), 1.69-1.46 (m, 5H). LCMS m/z 406.6 [M + H]⁺ |
| 53 | From S4^{1,2} 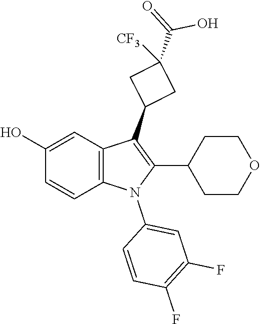 [SMILES: O=C(O)[C@]1(C(F)(F)F)C[C@@H](c2c(C3CCOCC3)n(-c3ccc(F)c(F)c3)c3ccc(O)cc32)C1] | 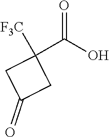 [SMILES: O=C1CC(C(=O)O)(C(F)(F)F)C1] | ¹H NMR (400 MHz, MeOD- d) δ 7.59-7.44 (m, 1H), 7.37 (ddd, J = 11.0, 7.2, 2.4 Hz, 1H), 7.19 (dd, J = 10.3, 1.8 Hz, 1H), 6.74-6.54 (m, 2H), 6.43 (s, 1H), 4.18 (dt, J = 14.6, 9.5 Hz, 1H), 3.93 (d, J = 11.2 Hz, 2H), 3.44-3.33 (m, 2H), 3.15 (td, J = 10.3, 2.8 Hz, 1H), 2.97-2.67 (m, 2H), 1.76 (dq, J = 8.0, 3.7, 3.2 Hz, 3H). LCMS m/z 496.5 [M + H]⁺ 540.21 (M + 1)⁺ |
| 54 | From S6^{1,4} 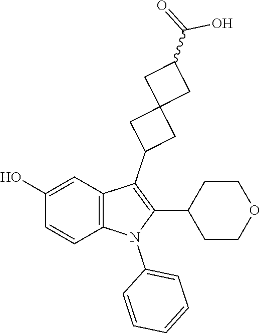 [SMILES: O=C(O)C1CC2(C1)CC(c1c(C3CCOCC3)n(-c3ccccc3)c3ccc(O)cc13)C2] | 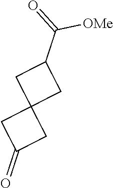 [SMILES: COC(=O)C1CC2(CC(=O)C2)C1] | ¹H NMR (400 MHz, DMSO- d6) δ 12.07 (s, 1H), 8.77 (s, 1H), 7.66-7.44 (m, 3H), 7.39- 7.23 (m, 2H), 7.11 (d, J = 2.2 Hz, 1H), 6.59 (s, 1H), 6.52 (dd, J = 8.8, 2.2 Hz, 1H), 3.91-3.60 (m, 3H), 3.13 (t, J = 11.6 Hz, 2H), 3.04-3.01 (m, 1H), 2.79-2.58 (m, 2H), 2.53-2.49 (m, 3H), 2.38-2.22 (m, 4H), 1.87-1.84 (m, 2H), 1.58 (d, J = 12.9 Hz, 2H). LCMS m/z 432.5 [M + H]⁺ |
| 55 | From S5^{1,2,3} 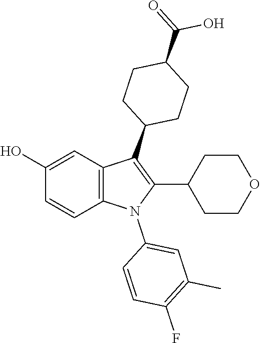 [SMILES: Cc1cc(-n2c(C3CCOCC3)c([C@H]3CC[C@@H](C(=O)O)CC3)c3cc(O)ccc32)ccc1F] | 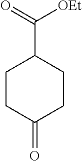 [SMILES: CCOC(=O)C1CCC(=O)CC1] | ¹H NMR (400 MHz, Methanol-d4) δ 7.25-7.13 (m, 2H), 7.13-7.02 (m, 2H), 6.59 (d, J = 8.7 Hz, 1H), 6.53 (dd, J = 8.7, 2.3 Hz, 1H), 3.95 (dd, J = 11.6, 4.1 Hz, 2H), 3.30-3.28 (m, 2H), 3.06 (m, 1H), 2.83-2.81 (m, 2H), 2.42- 2.27 (m, 7H), 2.18-1.96 (m, 2H), 1.80-1.60 (m, 6H). LCMS m/z 452.6 [M + H]⁺ |
| 56 | From S5^{1,2,3} 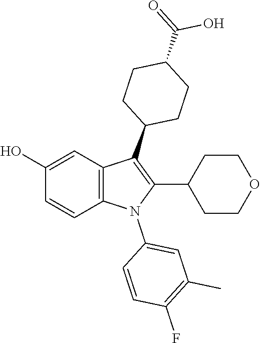 [SMILES: Cc1cc(-n2c(C3CCOCC3)c([C@H]3CC[C@H](C(=O)O)CC3)c3cc(O)ccc32)ccc1F] | 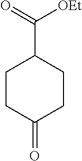 [SMILES: CCOC(=O)C1CCC(=O)CC1] | ¹H NMR (400 MHz, Methanol-d4) δ 7.26-7.14 (m, 2H), 7.10 (dd, J = 8.5, 2.6 Hz, 2H), 6.61 (d, J = 8.7 Hz, 1H), 6.55 (dd, J = 8.7, 2.3 Hz, 1H), 3.96 (dd, J = 11.5, 4.1 Hz, 2H), 3.30-3.30 (m, 2H), 3.05 (m, 1H), 2.90-2.74 (m, 1H), 2.55-2.45 (m, 1H), 2.34 (d, J = 1.9 Hz, 3H), 2.23- 1.98 (m, 7H), 1.88-1.86 (m, 2H), 1.70-1.55 (m, 4H). LCMS m/z 452.6 [M + H]⁺ |
| 57 | From S4^{1,3,4,6} 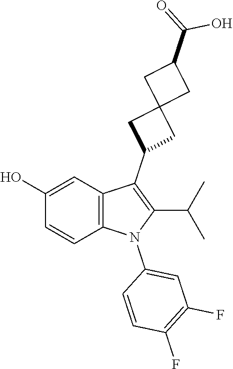 | 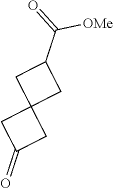 [SMILES: COC(=O)C1CC2(CC(=O)C2)C1] | ¹H NMR (400 MHz, Methanol-d4) δ 7.44 (dt, J = 10.5, 8.8 Hz, 1H), 7.25 (ddd, J = 10.9, 7.1, 2.5 Hz, 1H), 7.20 (d, J = 2.2 Hz, 1H), 7.11- 7.09 (m 1H), 6.64 (d, J = 8.7 Hz, 1H), 6.57 (dd, J = 8.7, 2.3 Hz, 1H), 3.86-3.73 (m, 1H), 3.09 (p, J = 8.5 Hz, 1H), 2.95- 2.93 (m, 1H), 2.74-2.72 (m, 2H), 2.54-2.34 (m, 6H), 2.28 (ddd, J = 11.1, 8.4, 4.7 Hz, 1H), 1.28-1.25 (m, 4H), 1.25 (d, J = 2.4 Hz, 3H). LCMS m/z 426.6 [M + H]⁺ |
| 58 | From S4^{1,3,4,6} 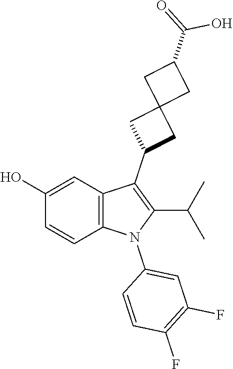 | 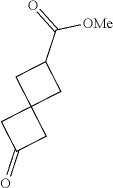 [SMILES: COC(=O)C1CC2(CC(=O)C2)C1] | ¹H NMR (400 MHz, Methanol-d4) δ 7.45-7.35 (m, 1H), 7.29-7.14 (m, 2H), 7.08 (d, J = 8.5 Hz, 1H), 6.65-6.62 (m, 1H), 6.60-6.52 (m, 1H), 3.80 (p, J = 9.3 Hz, 1H), 3.09 (p, J = 8.5 Hz, 1H), 2.95-2.91 (m, 1H), 2.73-2.77 (m, 2H), 2.56-2.34 (m, 5H), 2.31-2.28 (m, 1H), 1.25 (d, J = 6.8 Hz, 6H). LCMS m/z 426.2 [M + H]⁺ |
| 59 | From S8^{1,3,4} 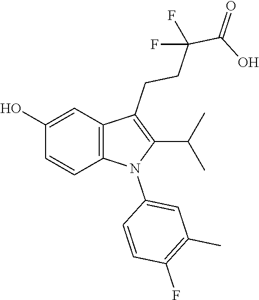 [SMILES: Cc1cc(-n2c(C(C)C)c(CCC(F)(F)C(=O)O)c3cc(O)ccc32)ccc1F] | 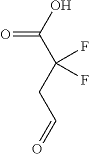 | ¹H NMR (400 MHz, Methanol-d4) δ 7.25-7.16 (m, 2H), 7.12 (dd, J = 4.9, 2.9 Hz, 1H), 6.90 (d, J = 2.3 Hz, 1H), 6.61 (d, J = 8.6 Hz, 1H), 6.54 (dd, J = 8.7, 2.3 Hz, 1H), 2.98-2.96 (m, 3H), 2.33-2.29 (m, 5H), 1.29 (d, J = 8 Hz, 6H). LCMS m/z 406.5 [M + H]⁺ |
| 60 | From S8^{1,2} 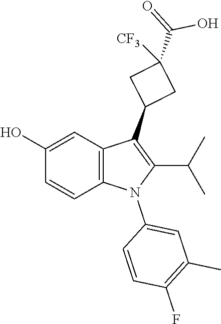 [SMILES: Cc1cc(-n2c(C(C)C)c([C@H]3C[C@@](C(=O)O)(C(F)(F)F)C3)c3cc(O)ccc32)ccc1F] | 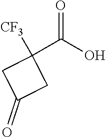 [SMILES: O=C1CC(C(=O)O)(C(F)(F)F)C1] | ¹H NMR (400 MHz, Chloroform-d) δ 7.38-7.32 (m, 1H), 7.22-7.01 (m, 3H), 6.76 (d, J = 8.7 Hz, 1H), 6.69 (dd, J = 8.7, 2.3 Hz, 1H), 4.34- 4.15 (m, 1H), 3.29 (ddd, J = 12.9, 10.7, 2.3 Hz, 2H), 2.99 (p, J = 7.2 Hz, 1H), 2.87 (td, J = 9.2, 2.8 Hz, 2H), 2.36 (d, J = 1.9 Hz, 3H), 1.29 (dd, J = 7.2, 2.0 Hz, 6H). LCMS m/z 450.0 [M + H]⁺ |
| 61 | From S8^{1,2} 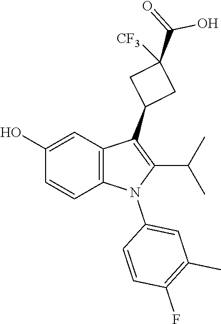 [SMILES: Cc1cc(-n2c(C(C)C)c([C@H]3C[C@](C(=O)O)(C(F)(F)F)C3)c3cc(O)ccc32)ccc1F] | 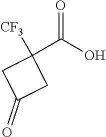 [SMILES: O=C1CC(C(=O)O)(C(F)(F)F)C1] | ¹H NMR (400 MHz, Chloroform-d) δ 7.63 (d, J = 2.2 Hz, 1H), 7.22-7.01 (m, 3H), 6.80-6.66 (m, 2H), 4.25- 4.06 (m, 1H), 3.41 (t, J = 11.6 Hz, 2H), 3.02-2.88 (m, 2H), 2.88-2.77 (m, 3H), 2.34 (d, J = 1.9 Hz, 3H), 1.27 (dd, J = 7.2, 2.0 Hz, 6H). LCMS m/z 450.2 [M + H]⁺ |
| 62 | From S8^{1,3,4} 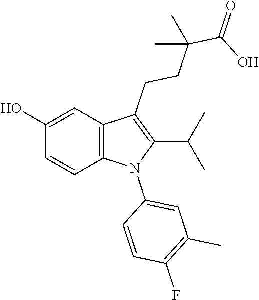 [SMILES: Cc1cc(-n2c(C(C)C)c(CCC(C)(C)C(=O)O)c3cc(O)ccc32)ccc1F] | 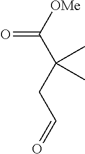 | ¹H NMR (400 MHz, DMSO- d6) δ 12.27 (s, 1H), 8.71 (s, 1H), 7.38-7.25 (m, 2H), 7.19-7.16 (m, 1H), 6.80 (d, J = 2.2 Hz, 1H), 6.58 (d, J = 8.6 Hz, 1H), 6.51 (dd, J = 8.6, 2.3 Hz, 1H), 2.92-2.77 (m, 1H), 2.72-2.62 (m, 2H), 2.30 (d, J = 1.9 Hz, 3H), 1.83-1.65 (m, 2H), 1.24-1.22 (m, 12H). LCMS m/z 398.2 [M + H]⁺ |
| 63 | From S8^{1,2,5} 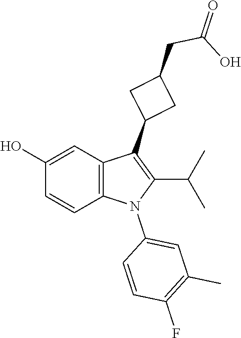 [SMILES: Cc1cc(-n2c(C(C)C)c([C@H]3C[C@@H](CC(=O)O)C3)c3cc(O)ccc32)ccc1F] | 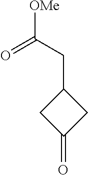 | ¹H NMR (400 MHz, Methanol-d4) δ 7.36-7.30 (m, 1H), 7.23-6.99 (m, 3H), 6.65-6.46 (m, 2H), 3.89- 3.71 (m, 1H), 2.95 (p, J = 7.2 Hz, 1H), 2.75-2.57 (m, 3H), 2.45 (dd, J = 12.1, 8.6 Hz, 4H), 2.33 (d, J = 2.0 Hz, 3H), 1.26 (dd, J = 7.2, 0.8 Hz, 7H). LCMS m/z 396.0 [M + H]⁺ |
| 64 | From S8^{1,2,5,12} 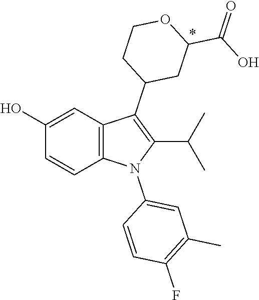 [SMILES: Cc1cc(-n2c(C(C)C)c(C3CCOC(C(=O)O)C3)c3cc(O)ccc32)ccc1F] | 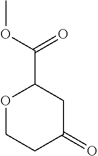 | ¹H NMR (400 MHz, Methanol-d4) δ 7.23-7.04 (m, 3H), 6.62 (dd, J = 8.7, 0.5 Hz, 1H), 6.54 (dd, J = 8.7, 2.3 Hz, 1H), 4.60 (dd, J = 6.0, 1.4 Hz, 1H), 4.05-3.89 (m, 2H), 2.99-2.82 (m, 1H), 2.58 (d, J = 6.0 Hz, 1H), 2.33 (t, J = 1.8 Hz, 3H), 2.28-2.19 (m, 1H), 1.70-1.58 (m, 1H), 1.36-1.27 (m, 5H). LCMS m/z 412.0 [M + H]⁺ |
| 65 | From S8^{1,2,5,12} 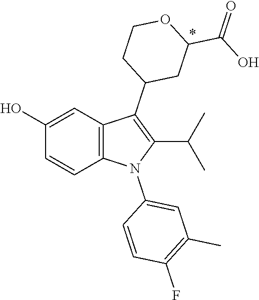 [SMILES: Cc1cc(-n2c(C(C)C)c(C3CCOC(C(=O)O)C3)c3cc(O)ccc32)ccc1F] | 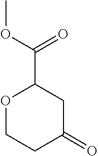 | ¹H NMR (400 MHz, Methanol- d4) δ 7.27-7.05 (m, 4H), 6.62 (d, J = 8.7 Hz, 1H), 6.54 (dt, J = 8.8, 1.5 Hz, 1H), 4.60 (d, J = 5.8 Hz, 1H), 3.99 (dd, J = 11.7, 8.6 Hz, 2H), 3.30 (p, J = 1.6 Hz, 4H), 2.96-2.85 (m, 1H), 2.58 (d, J = 6.0 Hz, 0H), 2.26 (s, 0H), 1.65 (d, J = 13.3 Hz, 1H), 1.36-1.21 (m, 7H). LCMS m/z 412.0 [M + H]⁺ |
| 66 | From S8^{1,3,4} 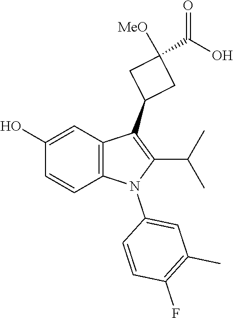 [SMILES: CO[C@]1(C(=O)O)C[C@H](c2c(C(C)C)n(-c3ccc(F)c(C)c3)c3ccc(O)cc32)C1] | 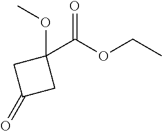 | ¹H NMR (400 MHz, Methanol-d4) δ 7.37 (d, J = 2.2 Hz, 1H), 7.24-7.18 (m, 1H), 7.18-7.14 (m, 1H), 7.12- 7.06 (m, 1H), 6.64 (d, J = 8.7 Hz, 1H), 6.60-6.55 (m, 1H), 3.97-3.84 (m, 1H), 3.39 (s, 3H), 2.98-2.86 (m, 3H), 2.83-2.75 (m, 2H), 2.34 (d, J = 1.8 Hz, 3H), 1.28 (d, J = 7.2 Hz, 6H). LCMS m/z 412.0 [M + H]⁺ |
| 67 | From S8^{1,3,4} 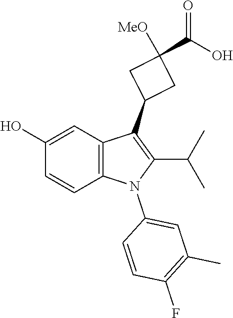 [SMILES: CO[C@]1(C(=O)O)C[C@@H](c2c(C(C)C)n(-c3ccc(F)c(C)c3)c3ccc(O)cc32)C1] | 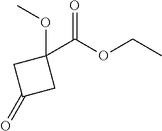 | ¹H NMR (400 MHz, Methanol-d4) δ 7.46 (d, J = 1.9 Hz, 1H), 7.25-7.14 (m, 2H), 7.12-7.05 (m, 1H), 6.64- 6.60 (m, 1H), 6.57 (dd, J = 8.7, 2.2 Hz, 1H), 4.20-4.06 (m, 1H), 3.43 (s, 3H), 3.22- 3.11 (m, 2H), 3.00-2.90 (m, 1H), 2.54-2.44 (m, 2H), 2.34 (d, J = 1.9 Hz, 3H), 1.28 (d, J = 7.2 Hz, 6H). LCMS m/z 412.0 [M + H]⁺ |
| 68 | From S19^{1,3,8} 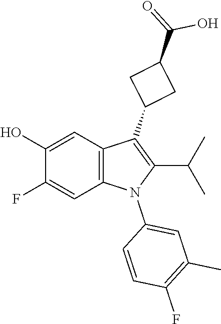 [SMILES: Cc1cc(-n2c(C(C)C)c([C@H]3C[C@H](C(=O)O)C3)c3cc(O)c(F)cc32)ccc1F] | 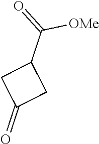 | ¹H NMR (400 MHz, Chloroform-d) δ 7.48 (d, J = 8.4 Hz, 1H), 7.20-6.92 (m, 3H), 6.63 (d, J = 10.9 Hz, 1H), 4.39-4.22 (m, 1H), 3.52- 3.39 (m, 1H), 3.07-3.05 (m,, 2H), 2.95 (p, J = 7.2 Hz, 1H), 2.747-2.74 (m, 2H), 2.36 (d, J = 2.0 Hz, 3H), 1.27 (d, J = 4 Hz, 3H), 1.25 (d, J = 4 Hz, 3H). LCMS m/z 400.0 [M + H]⁺ |
| 69 | From S19^{1,3,8} 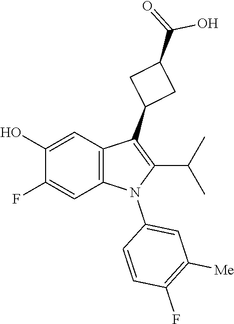 [SMILES: Cc1cc(-n2c(C(C)C)c([C@H]3C[C@@H](C(=O)O)C3)c3cc(O)c(F)cc32)ccc1F] | 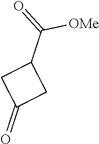 | ¹H NMR (400 MHz, Chloroform-d) δ 7.79 (d, J = 8.5 Hz, 1H), 7.19-6.95 (m, 3H), 6.60 (d, J = 11.0 Hz, 1H), 4.02-3.79 (m, 1H), 3.32- 3.18 (m, 1H), 3.16-3.02 (m, 2H), 2.95 (p, J = 7.2 Hz, 1H), 2.68-2.51 (m, 2H), 2.35 (d, J = 2.0 Hz, 3H), 1.27 (d, J = 4 Hz, 3H), 1.26 (d, J = 4 Hz, 3H). LCMS m/z 400.1 [M + H]⁺ |
| 70 | From S11^{1,3,4} 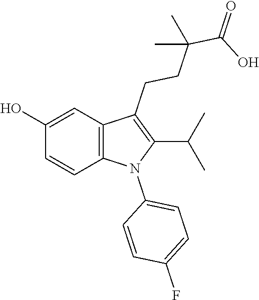 [SMILES: CC(C)c1c(CCC(C)(C)C(=O)O)c2cc(O)ccc2n1-c1ccc(F)cc1] | 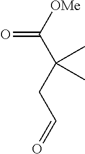 | ¹H NMR (400 MHz, Chloroform-d) δ 7.32-7.24 (m, 2H), 7.21 (dd, J = 8.9, 8.1 Hz, 2H), 7.02 (dd, J = 2.4, 0.6 Hz, 1H), 6.73 (dd, J = 8.7, 0.6 Hz, 1H), 6.66 (dd, J = 8.7, 2.4 Hz, 1H), 3.02-2.89 (m, 1H), 2.87-2.77 (m, 2H), 2.01- 1.92 (m, 2H), 1.40 (s, 6H), 1.28 (d, J = 7.2 Hz, 6H). LCMS m/z 384.0 [M + H]⁺ |
| 71 | From S11^{1,3,4} 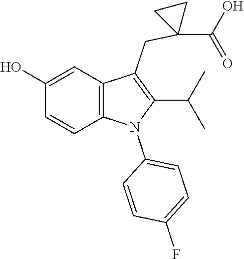 [SMILES: CC(C)c1c(CC2(C(=O)O)CC2)c2cc(O)ccc2n1-c1ccc(F)cc1] | 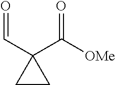 | ¹H NMR (400 MHz, Chloroform-d) δ 7.35-7.27 (m, 2H), 7.27-7.18 (m, 2H), 6.97 (dd, J = 2.0, 1.0 Hz, 1H), 6.65 (t, J = 1.5 Hz, 2H), 3.49 (s, 2H), 3.18-3.05 (m, 1H), 1.27 (q, J = 3.8 Hz, 2H), 1.21 (d, J = 7.2 Hz, 6H), 0.86 (q, J = 4.0 Hz, 2H). LCMS m/z 368.0 [M + H]⁺ |
| 72 | From S13^{9,8} 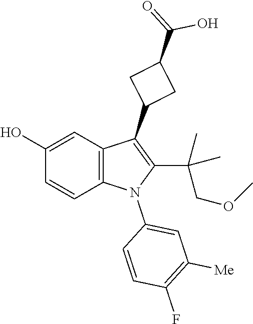 | 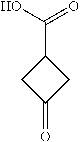 | ¹H NMR (400 MHz, DMSO- d6) δ 12.12 (s, 1H), 8.51 (s, 1H), 7.33 (t, J = 9.0 Hz, 1H), 7.28 (dd, J = 6.9, 2.6 Hz, 1H), 7.22 (dd, J = 8.1, 4.5 Hz, 1H), 6.70 (s, 1H), 6.51 (d, J = 8.6 Hz, 1H), 6.18 (d, J = 8.6 Hz, 1H), 3.97 (t, J = 9.1 Hz, 1H), 3.14 (s, 3H), 3.12 (d, J = 1.9 Hz, 2H), 3.11-3.03 (m, 1H), 2.83 (q, J = 10.4 Hz, 2H), 2.52-2.45 (d, J = 10.7 Hz, 2H), 2.30 (d, J = 1.8 Hz, 3H), 1.21 (s, 3H), 1.20 (s, 3H). LCMS m/z 426.3 [M + H]⁺ |
| 73 | From S13^{9,8} 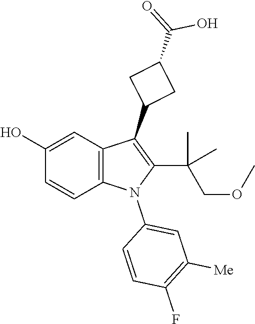 | 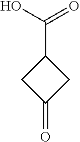 | ¹H NMR (400 MHz, DMSO- d6) δ 12.18-12.07 (m, 1H), 8.57 (s, 1H), 7.33 (t, J = 8.9 Hz, 1H), 7.29-7.24 (m, 1H), 7.21 (s, 1H), 6.53 (d, J = 8.5 Hz, 1H), 6.40 (s, 1H), 6.19 (d, J = 8.7 Hz, 1H), 4.12 (t, J = 9.3 Hz, 1H), 3.16-3.10 (m, 6H), 2.88 (q, J = 9.7 Hz, 2H), 2.52-2.48 (m, 2H), 2.30 (s, 3H), 1.21 (s, 3H), 1.19 (s, 3H). LCMS m/z 426.3 [M + H]⁺ |
| 74 | From S8^{1,3,4,12} 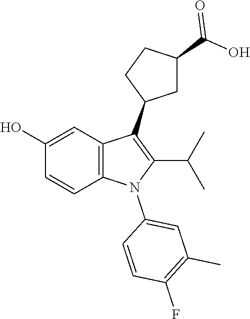 [SMILES: Cc1cc(-n2c(C(C)C)c([C@H]3CC[C@@H](C(=O)O)C3)c3cc(O)ccc32)ccc1F] | 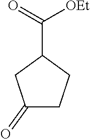 [SMILES: CCOC(=O)C1CCC(=O)C1] | ¹H NMR (400 MHz, Chloroform-d) δ 7.24-7.22 (m, 1H), 7.16-6.98 (m, 3H), 6.73 (s, 1H), 6.66-6.64 (m, 1H), 3.54-3.51 (m, 1H), 3.07- 3.04 (m, 1H), 2.91 (p, J = 7.2 Hz, 1H), 2.62-2.46 (m, 1H), 2.42-2.23 (m, 6H), 2.16- 1.93 (m, 2H), 1.27-1.22 (m, 6H). LCMS m/z 396.1 [M + H]⁺. |
| 75 | From S9^{1,2} 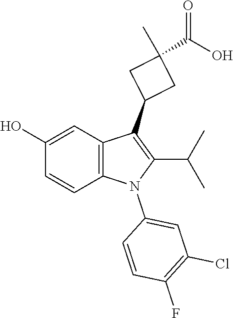 [SMILES: CC(C)c1c([C@H]2C[C@](C)(C(=O)O)C2)c2cc(O)ccc2n1-c1ccc(F)c(Cl)c1] | 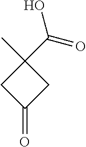 | ¹H NMR (400 MHz, Methanol-d4) δ 7.57-7.41 (m, 3H), 7.35-7.30 (m, 1H), 6.61-6.56 (m, 2H), 4.16- 3.99 (m, 1H), 3.15 (q, J = 9.0, 8.6 Hz, 1H), 3.01-2.84 (m, 3H), 2.66-2.53 (m, 2H), 1.22 (d, J = 6.8 Hz, 6H). LCMS m/z 416.0 [M + H]⁺. |
| 76 | From S9^{1,2} 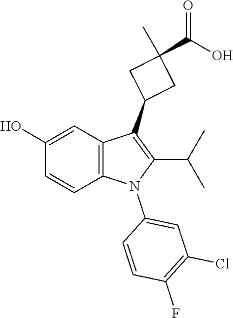 [SMILES: CC(C)c1c([C@H]2C[C@@](C)(C(=O)O)C2)c2cc(O)ccc2n1-c1ccc(F)c(Cl)c1] | 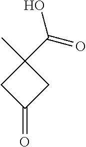 | ¹H NMR (400 MHz, Chloroform-d) δ 7.75-7.67 (m, 1H), 7.37 (dd, J = 6.5,2.5 Hz, 1H), 7.31-7.26 (m, 2H), 7.19 (ddd, J = 8.7, 4.3, 2.5 Hz, 1H), 6.80-6.70 (m, 2H), 4.03 (q, J = 9.4 Hz, 1H), 3.32 (t, J = 10.8 Hz, 2H), 2.94 (p, J = 7.2 Hz, 1H), 2.23 (dd, J = 12.0, 8.7 Hz, 2H), 1.68 (s, 3H), 1.34-1.29 (m, 6H). LCMS m/z 416.7 [M + H]⁺. |
| 77 | From S9^{1,2} 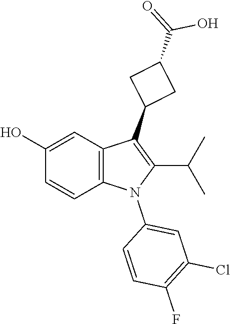 [SMILES: CC(C)c1c([C@H]2C[C@H](C(=O)O)C2)c2cc(O)ccc2n1-c1ccc(F)c(Cl)c1] | 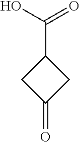 | ¹H NMR (400 MHz, Methanol-d4) δ 7.50-7.38 (m, 2H), 7.33 (dd, J = 2.3, 0.6 Hz, 1H), 7.25 (ddd, J = 8.7, 4.2, 2.5 Hz, 1H), 6.70-6.54 (m, 2H), 4.14-3.90 (m, 1H), 2.91 (p, J = 7.2 Hz, 1H), 2.80- 2.60 (m, 4H), 1.60 (s, 3H), 1.27 (d, J = 7.2 Hz, 6H). LCMS m/z 402.7 [M + H]⁺. |
| 78 | From S9^{1,2} 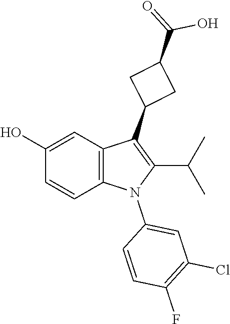 [SMILES: CC(C)c1c([C@H]2C[C@@H](C(=O)O)C2)c2cc(O)ccc2n1-c1ccc(F)c(Cl)c1] | 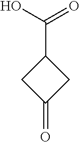 | ¹H NMR (400 MHz, Chloroform-d) δ 7.75 (t, J = 1.4 Hz, 1H), 7.38 (dd, J = 6.5, 2.5 Hz, 1H), 7.31 (d, J = 8.6 Hz, 1H), 7.24-7.15 (m, 1H), 6.77 (d, J = 1.4 Hz, 2H), 3.96 (t, J = 9.5 Hz, 1H), 3.37- 3.08 (m, 3H), 2.99-2.87 (m, 1H), 2.61 (dt, J = 11.7, 8.4 Hz, 2H), 1.37-1.21 d, 6H). LCMS m/z 402.7 [M + H]⁺. |
| 79 | From S8^{1,3,4,12} 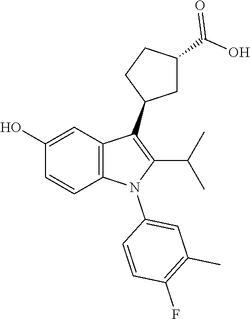 [SMILES: Cc1cc(-n2c(C(C)C)c([C@H]3CC[C@H](C(=O)O)C3)c3cc(O)ccc32)ccc1F] | 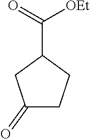 [SMILES: CCOC(=O)C1CCC(=O)C1] | ¹H NMR (400 MHz, Chloroform-d) δ 7.19-7.06 (m, 3H), 7.03 (d, J = 2.4 Hz, 1H), 6.77 (d, J = 8.7 Hz, 1H), 6.63 (dd, J = 8.7, 2.4 Hz, 1H), 3.81-3.62 (m, 1H), 3.34- 3.21 (m, 1H), 2.98-2.95 (m, 1H), 2.52-2.16 (m, 6H), 2.15- 2.04 (m, 1H), 1.33-1.29 (m, 6H). LCMS m/z 396.4 [M + H]⁺. |
| 80 | From S11^{9,8,12} 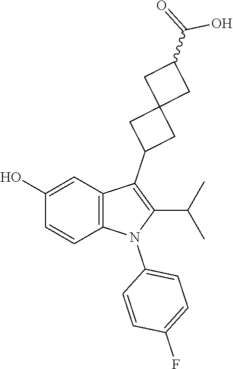 [SMILES: CC(C)c1c(C2CC3(CC(C(=O)O)C3)C2)c2cc(O)ccc2n1-c1ccc(F)cc1] | 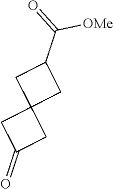 [SMILES: COC(=O)C1CC2(CC(=O)C2)C1] | ¹H NMR (300 MHz, DMSO- d6) δ 7.37 (d, J = 7.0 Hz, 4H), 7.14 (d, J = 2.1 Hz, 1H), 6.58- 6.47 (m, 2H), 3.70 (p, J = 9.3 Hz, 1H), 3.35 (s, 2H), 2.92-2.78 (m, 1H), 2.60 (t, J = 8.9 Hz, 3H), 2.41-2.28 (m, 1H), 2.28-2.15 (m, 3H), 2.08 (d, J = 2.9 Hz, 2H), 1.20 (d, J = 2.7 Hz, 3H), 1.17 (d, J = 2.7 Hz, 3H). LCMS m/z 408.3 [M + H]⁺ |
| 81 | From S13^{9,4,3} 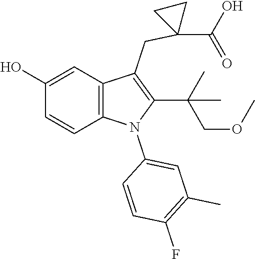 [SMILES: COCC(C)(C)c1c(CC2(C(=O)O)CC2)c2cc(O)ccc2n1-c1ccc(F)c(C)c1] | 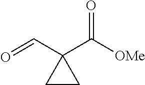 | ¹H NMR (400 MHz, DMSO- d6) δ 8.72 (s, 1H), 7.30 (t, J = 9.0 Hz, 1H), 7.25 (dd, J = 7.0, 2.6 Hz, 1H), 7.19 (dd, J = 8.3, 4.5 Hz, 1H), 6.72 (d, J = 2.3 Hz, 1H), 6.47 (dd, J = 8.7, 2.3 Hz, 1H), 6.21 (d, J = 8.7 Hz, 1H), 3.47 (d, J = 2.2 Hz, 2H), 3.13 (s, 3H), 2.29 (d, J = 1.8 Hz, 2H), 1.27 (s, 3H), 1.25 (s, 3H), 0.97 (d, J = 3.1 Hz, 2H), 0.65 (q, J = 10.4, 9.9 Hz, 2H). LCMS m/z 426.3 [M + H]⁺ |
| 82 | From S8^{1,13,10,2,12} 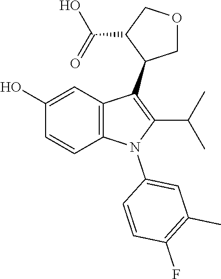 | 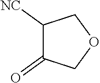 | ¹H NMR (400 MHz, Chloroform-d) δ 7.24-7.03 (m, 4H), 6.74 (d, J = 8.7 Hz, 1H), 6.67 (dt, J = 8.9, 1.7 Hz, 1H), 4.52 (t, J = 8.6 Hz, 1H), 4.22 (dq, J = 22.8, 6.0 Hz, 4H), 3.69 (q, J = 7.3 Hz, 1H), 3.03 (p, J = 7.2 Hz, 1H), 2.35 (s, 3H), 1.26 (d, J = 7.0 Hz, 6H). LCMS m/z 398.8 [M + H]⁺ |
| 83 | From S8^{1,13,10,2,12} 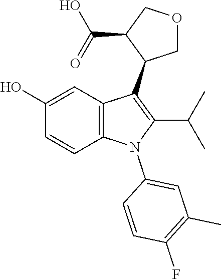 [SMILES: Cc1cc(-n2c(C(C)C)c([C@@H]3COC[C@@H]3C(=O)O)c3cc(O)ccc32)ccc1F] | 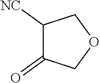 | ¹H NMR (400 MHz, Chloroform-d) δ 7.16 (d, J = 8.2 Hz, 4H), 6.77 (d, J = 8.6 Hz, 1H), 6.71 (d, J = 8.7 Hz, 1H), 6.46 (s, 1H), 4.61 (t, J = 8.9 Hz, 1H), 4.46-4.35 (m, 2H), 4.19 (t, J = 7.9 Hz, 1H), 4.06 (t, J = 8.7 Hz, 1H), 3.61 (q, J = 7.8 Hz, 1H), 2.91 (p, J = 6.8 Hz, 1H), 2.36 (s, 3H), 1.22 (s, 6H). LCMS m/z 398.7 [M + H]⁺ |
| 84 | From S15^{6,1,2,5} 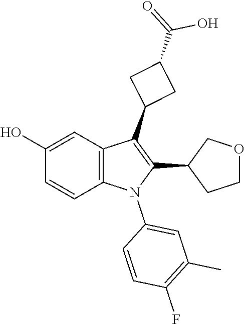 [SMILES: Cc1cc(-n2c([C@@H]3CCOC3)c([C@H]3C[C@H](C(=O)O)C3)c3cc(O)ccc32)ccc1F] | 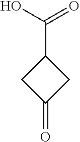 | ¹H NMR (400 MHz, Methanol-d4) δ 7.33 (dd, J = 5.9, 2.2 Hz, 1H), 7.28-7.04 (m, 3H), 6.70 (d, J = 8.8 Hz, 1H), 6.65-6.52 (m, 2H), 4.28- 4.14 (m, 1H), 4.06 (td, J = 8.4, 4.2 Hz, 1H), 3.92 (t, J = 8.7 Hz, 1H), 3.84-3.71 (m, 2H), 3.06-2.94 (m, 3H), 2.70- 2.57 (m, 3H), 2.33 (d, J = 2.1 Hz, 3H), 2.27-1.92 (m, 2H). LCMS m/z 410.7 [M + H]⁺. |
| 85 | From S15^{6,1,2,5} 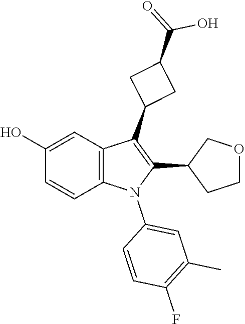 [SMILES: Cc1cc(-n2c([C@@H]3CCOC3)c([C@H]3C[C@@H](C(=O)O)C3)c3cc(O)ccc32)ccc1F] | 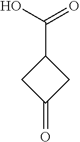 | ¹H NMR (400 MHz, Methanol-d4) δ 7.57-7.47 (m, 1H), 7.27-7.04 (m, 3H), 6.68 (d, J = 8.7 Hz, 1H), 6.59 (dd, J = 8.8, 2.3 Hz, 1H), 4.09 (d, J = 3.5 Hz, 1H), 3.99- 3.79 (m, 3H), 3.71 (q, J = 8.4 Hz, 1H), 3.27-3.17 (m, 1H), 3.07-2.86 (m, 2H), 2.55- 2.45 (m, 2H), 2.33 (s, 3H), 2.29-2.20 (m, 1H), 2.12-2.05 (m, 1H). LCMS m/z 410.7 [M + H]⁺. |
| 86 | From S15^{6,1,2,5} 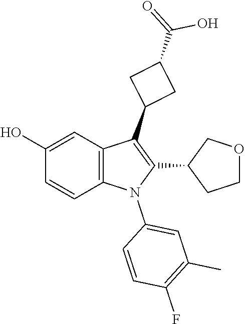 [SMILES: Cc1cc(-n2c([C@H]3CCOC3)c([C@H]3C[C@H](C(=O)O)C3)c3cc(O)ccc32)ccc1F] | 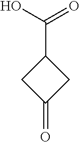 | ¹H NMR (400 MHz, Methanol-d4) δ 7.33 (d, J = 2.2 Hz, 1H), 7.28-7.04 (m, 4H), 6.70 (d, J = 8.7 Hz, 1H), 6.61 (dd, J = 8.7, 2.3 Hz, 1H), 4.26-4.14 (m, 1H), 4.07 (td, J = 8.4, 4.1 Hz, 1H), 3.93 (t, J = 8.7 Hz, 1H), 3.81-3.70 (m, 2H), 3.00 (dtd, J = 14.9, 9.7, 5.3 Hz, 2H), 2.60 (dtt, J = 12.4, 6.7, 3.2 Hz, 2H), 2.40- 2.31 (m, 4H), 2.30-2.18 (m, 1H), 2.17-1.98 (m, 1H). LCMS m/z 410.7 [M + H]⁺. |
| 87 | From S15^{6,1,2,5} 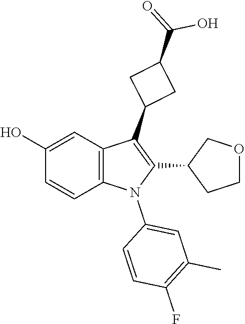 [SMILES: Cc1cc(-n2c([C@H]3CCOC3)c([C@H]3C[C@@H](C(=O)O)C3)c3cc(O)ccc32)ccc1F] | 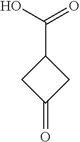 | ¹H NMR (400 MHz, Methanol-d4) δ 7.51 (d, J = 2.2 Hz, 1H), 7.30-7.05 (m, 4H), 6.68 (d, J = 8.7 Hz, 1H), 6.64-6.55 (m, 1H), 4.09 (q, J = 7.2 Hz, 2H), 4.00-3.81 (m, 3H), 3.71 (td, J = 8.8, 6.5 Hz, 1H), 3.18 (tt, J = 9.8, 8.2 Hz, 1H), 3.07-2.90 (m, 2H), 2.57- 2.43 (m, 2H), 2.39-2.30 (m, 4H), 2.25 (dddd, J = 12.6, 9.7, 6.6, 3.4 Hz, 1H), 2.09 (ddd, J = 12.1, 8.4, 3.2 Hz, 1H), 1.34-1.27 (m, 1H). LCMS m/z 410.7 [M + H]⁺. |
| 88 | From S8^{1,5,2,12} 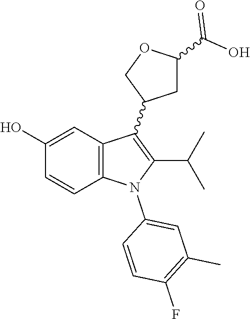 [SMILES: Cc1cc(-n2c(C(C)C)c(C3COC(C(=O)O)C3)c3cc(O)ccc32)ccc1F] | 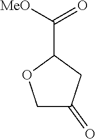 | ¹H NMR (400 MHz, Methanol-d4) δ 8.46 (s, 1H), 7.29-7.06 (m, 4H), 6.64- 6.47 (m, 3H), 4.55 (t, J = 8.3 Hz, 1H), 4.43 (t, J = 8.5 Hz, 1H), 4.20 (d, J = 9.2 Hz, 1H), 4.08 (t, J = 7.8 Hz, 1H), 2.88 (p, J = 6.8 Hz, 1H), 2.76- 2.51 (m, 2H), 2.34 (d, J = 1.9 Hz, 3H), 1.20 (d, J = 6.5 Hz, 6H). LCMS m/z 398.7 [M + H]⁺. |
| 89 | From S20^{1,4} 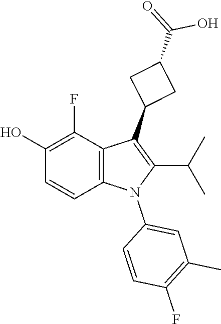 [SMILES: Cc1cc(-n2c(C(C)C)c([C@H]3C[C@H](C(=O)O)C3)c3c(F)c(O)ccc32)ccc1F] | 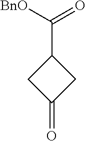 [SMILES: O=C1CC(C(=O)OCc2ccccc2)C1] | ¹H NMR (400 MHz, Chloroform-d) δ 7.13 (t, J = 8.7 Hz, 1H), 7.10-7.00 (m, 2H), 6.79 (t, J = 8.4 Hz, 1H), 6.51 (d, J = 8.7 Hz, 1H), 4.35 (p, J = 9.2 Hz, 1H), 3.35 (t, J = 10.1 Hz, 1H), 3.05-2.79 (m, 3H), 2.79-2.59 (m, 2H), 2.33 (s, 3H), 1.28 (dd, J = 7.3, 2.2 Hz, 6H) |
| 90 | From S17^{1,4} 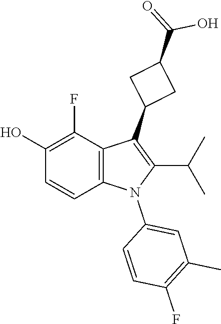 [SMILES: Cc1cc(-n2c(C(C)C)c([C@H]3C[C@@H](C(=O)O)C3)c3c(F)c(O)ccc32)ccc1F] | 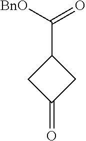 [SMILES: O=C1CC(C(=O)OCc2ccccc2)C1] | ¹H NMR (400 MHz, Chloroform-d) δ 7.20-7.02 (m, 3H), 6.79 (t, J = 8.4 Hz, 1H), 6.49 (d, J = 8.7 Hz, 1H), 4.04-3.80 (m, 1H), 3.26 (p, J = 9.2 Hz, 1H), 3.05-2.79 (m, 3H), 2.77-2.54 (m, 2H), 2.33 (d, J = 2.0 Hz, 3H), 1.37- 1.13 (m, 7H). LCMS m/z 400.8 [M + H]⁺. |
| 91 | From S11^{1,3,4} 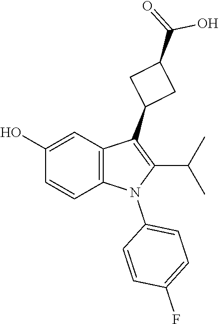 [SMILES: CC(C)c1c([C@H]2C[C@@H](C(=O)O)C2)c2cc(O)ccc2n1-c1ccc(F)cc1] | 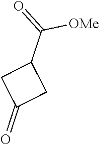 | ¹H NMR (400 MHz, Methanol-d4) δ 7.48 (dd, J = 2.2, 0.7 Hz, 1H), 7.31-7.25 (m, 4H), 6.58 (qd, J = 8.7, 1.4 Hz, 2H), 4.01-3.83 (m, 1H), 3.27-3.11 (m, 1H), 3.04- 2.85 (m, 3H), 2.57-2.45 (m, 2H), 1.27 (d, J = 7.3 Hz, 6H). LCMS m/z 366.7 [M + H]⁺. |
| 92 | From S11^{1,3,4} 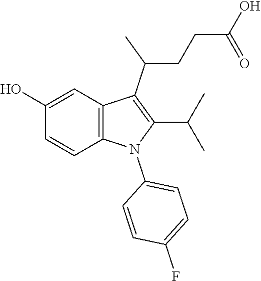 [SMILES: CC(C)c1c(C(C)CCC(=O)O)c2cc(O)ccc2n1-c1ccc(F)cc1] | 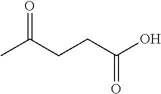 | ¹H NMR (400 MHz, Methanol-d4) δ 7.37-7.21 (m, 4H), 7.05 (dd, J = 2.3, 0.6 Hz, 1H), 6.62-6.51 (m, 2H), 3.27-3.19 (m, 1H), 2.99 (p, J = 7.3 Hz, 1H), 2.33-2.20 (m, 3H), 2.18-2.03 (m, 1H), 1.45 (d, J = 7.0 Hz, 3H), 1.26 (d, J = 7.3 Hz, 3H), 1.23 (d, J = 7.2 Hz, 3H). LCMS m/z 370.7 [M + H]⁺. |
| 93 | From S14^{1,3,7} 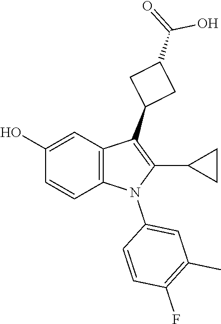 | 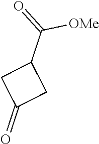 | ¹H NMR (400 MHz, Chloroform-d) δ 7.22 (d, J = 2.3 Hz, 1H), 7.18-7.08 (m, 3H), 7.03-6.92 (m, 1H), 6.70 (dd, J = 8.7, 2.4 Hz, 1H), 4.29 (tt, J = 9.7, 8.3 Hz, 1H), 3.48- 3.31 (m, 1H), 3.06-2.90 (m, 2H), 2.80 (tdd, J = 9.4, 3.8, 2.6 Hz, 2H), 2.39-2.27 (m, 3H), 1.73 (tt, J = 8.3, 5.3 Hz, 1H), 0.74-0.62 (m, 2H), 0.43-0.30 (m, 2H). LCMS m/z 379.5 [M + H]⁺. |
| 94 | From S8^{1,2} 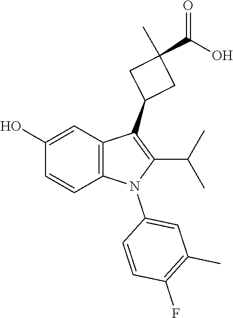 [SMILES: Cc1cc(-n2c(C(C)C)c([C@H]3C[C@@](C)(C(=O)O)C3)c3cc(O)ccc32)ccc1F] | 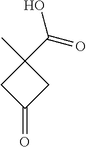 | ¹H NMR (400 MHz, Chloroform-d) δ 7.70 (d, J = 2.2 Hz, 1H), 7.20-7.02 (m, 3H), 6.79-6.67 (m, 2H), 4.52 (p, J = 7.3 Hz, 0H), 4.06 (ddd, J = 10.3, 8.8, 1.4 Hz, 1H), 3.34 (ddd, J = 12.4, 10.0, 2.3 Hz, 2H), 3.05-2.78 (m, 1H), 2.35 (d, J = 1.9 Hz, 3H), 2.22 (td, J = 9.0, 2.6 Hz, 2H), 1.99 (ddd, J = 10.1, 7.2, 2.8 Hz, 1H), 1.68 (s, 3H), 1.30 (dd, J = 7.2, 1.5 Hz, 6H). LCMS m/z 395.4 [M + H]⁺ |
| 95 | From S8^{1,2} 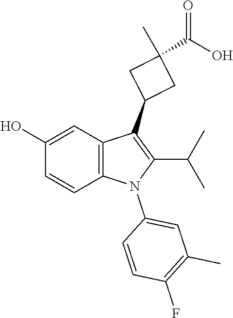 [SMILES: Cc1cc(-n2c(C(C)C)c([C@H]3C[C@](C)(C(=O)O)C3)c3cc(O)ccc32)ccc1F] | 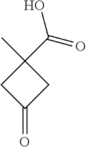 | ¹H NMR (400 MHz, Chloroform-d) δ 7.37 (d, J = 2.3 Hz, 1H), 7.24-7.06 (m, 3H), 6.75 (d, J = 8.7 Hz, 1H), 6.66 (dd, J = 8.7, 2.3 Hz, 1H), 4.25-4.06 (m, 1H), 3.07- 2.84 (m, 3H), 2.76 (tt, J = 10.3, 2.0 Hz, 2H), 2.35 (d, J = 1.9 Hz, 3H), 1.68 (s, 3H), 1.28 (dd, J = 7.2, 2.0 Hz, 6H). LCMS m/z 395.0 [M + H]⁺. |
| 96 | From S8^{1,3,4} 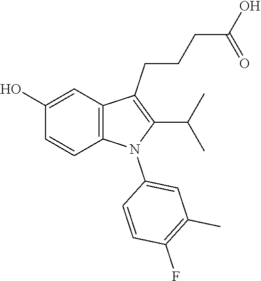 [SMILES: Cc1cc(-n2c(C(C)C)c(CCCC(=O)O)c3cc(O)ccc32)ccc1F] | 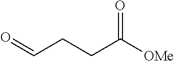 | ¹H NMR (400 MHz, DMSO- d6) δ 8.70 (s, 1H), 7.39-7.29 (m, 2H), 7.19-7.17(m,1H), 6.84 (d, J = 2.2 Hz, 1H), 6.63- 6.45 (m, 2H), 2.90 (p, J = 7.2 Hz, 1H), 2.80-2.66 (m, 2H), 2.38-2.36 (m, 2H), 2.30 (d, J = 2.0 Hz, 3H), 1.84-1.80 (m, 3H), 1.22 (d, J = 8.0 Hz, 6H). LCMS m/z 370.3 [M + H]⁺. |
| 97 | From S8^{1,3,4} 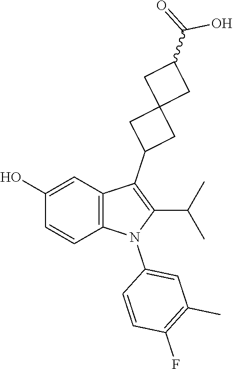 [SMILES: Cc1cc(-n2c(C(C)C)c(C3CC4(CC(C(=O)O)C4)C3)c3cc(O)ccc32)ccc1F] | 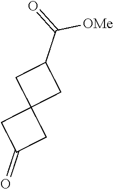 [SMILES: COC(=O)C1CC2(CC(=O)C2)C1] | ¹H NMR (400 MHz, DMSO- d6) δ 12.06 (s, 1H), 8.73 (s, 1H), 7.38-7.26 (m, 2H), 7.17 (d, J = 3.3 Hz, 1H), 7.09 (d, J = 2.2 Hz, 1H), 6.57 (s, 1H), 6.50 (dd, J = 8.7, 2.2 Hz, 1H), 3.73-3.71 (m, 1H), 3.03-3.01 (m, 1H), 2.93-2.80 (m, 1H), 2.71-2.54 (m, 2H), 2.46- 2.09 (m, 9H), 1.20-1.16 (m, 6H). LCMS m/z 421.2 [M + H]⁺ |
| 98 | From S8^{9,3,8} 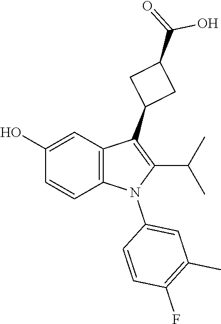 [SMILES: Cc1cc(-n2c(C(C)C)c([C@H]3C[C@@H](C(=O)O)C3)c3cc(O)ccc32)ccc1F] | 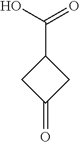 | ¹H NMR (300 MHz, Chloroform-d) δ 7.69 (dd, J = 2.1, 0.8 Hz, 1H), 7.09 (ddt, J = 10.5, 8.4, 7.0 Hz, 3H), 6.81- 6.62 (m, 2H), 3.94 (t, J = 9.3 Hz, 1H), 3.35-3.04 (m, 3H), 2.93 (p, J = 7.2 Hz, 1H), 2.58 (dt, J = 11.4, 8.0 Hz, 2H), 2.32 (d, J = 2.0 Hz, 3H), 1.27 (dd, J = 7.2, 1.0 Hz, 6H). LCMS m/z 382.3 [M + H]⁺. |
| 99 | From S5^{1,2} 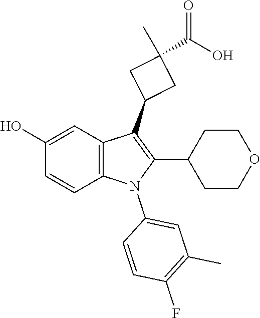 [SMILES: Cc1cc(-n2c(C3CCOCC3)c([C@H]3C[C@](C)(C(=O)O)C3)c3cc(O)ccc32)ccc1F] | 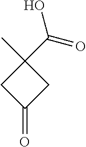 | ¹H NMR (400 MHz, Methanol-d4) δ 7.34 (dd, J = 2.2, 0.7 Hz, 1H), 7.26-7.13 (m, 2H), 7.08 (ddd, J = 8.1, 4.5, 2.7 Hz, 1H), 6.67-6.54 (m, 2H), 4.13-4.01 (m, 1H), 3.93 (dd, J = 11.6, 4.2 Hz, 2H), 3.26 (dd, J = 11.9, 2.0 Hz, 2H), 2.88-2.57 (m, 5H), 2.33 (d, J = 1.9 Hz, 3H), 2.03 (tdd, J = 12.6, 8.7, 3.8 Hz, 2H), 1.61 (s, 5H). LCMS m/z 438.7 [M + H]⁺. |
| 100 | From S5^{1,2} 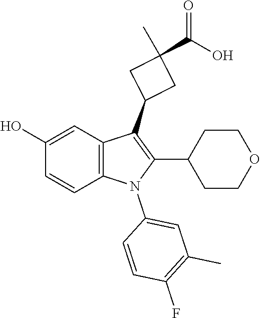 [SMILES: Cc1cc(-n2c(C3CCOCC3)c([C@H]3C[C@@](C)(C(=O)O)C3)c3cc(O)ccc32)ccc1F] | 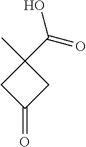 | ¹H NMR (400 MHz, Methanol-d4) δ 7.43 (d, J = 2.1 Hz, 1H), 7.28-7.00 (m, 4H), 6.70-6.52 (m, 3H), 4.13- 3.76 (m, 4H), 3.16 (t, J = 10.8 Hz, 2H), 2.98-2.59 (m, 3H), 2.33 (s, 3H), 2.27-1.92 (m, 4H), 1.75 (td, J = 9.7, 8.6, 3.6 Hz, 1H), 1.63 (d, J = 17.5 Hz, 5H). LCMS m/z 438.8 [M + H]⁺ |
| 101 | From S8^{1,3,7,6,14} 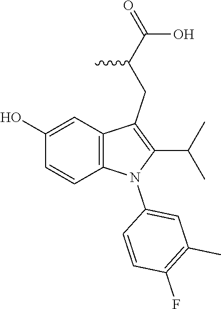 [SMILES: Cc1cc(-n2c(C(C)C)c(CC(C)C(=O)O)c3cc(O)ccc32)ccc1F] | 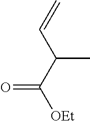 | ¹H NMR (300 MHz, Methanol-d4) δ 7.28-7.08 (m, 3H), 6.93 (t, J = 1.5 Hz, 1H), 6.54 (d, J = 1.4 Hz, 2H), 3.25-3.06 (m, 2H), 2.92- 2.74 (m, 2H), 2.33 (d, J = 2.1 Hz, 3H), 1.22 (t, J = 6.7 Hz, 6H), 1.16 (d, J = 6.2 Hz, 3H). LCMS m/z 370.4 [M + H]⁺. |
| 102 | From S8^{1,3,7,6,14} 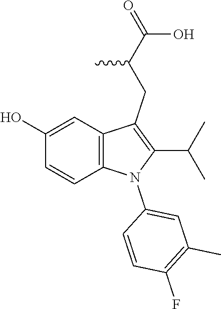 [SMILES: Cc1cc(-n2c(C(C)C)c(CC(C)C(=O)O)c3cc(O)ccc32)ccc1F] | 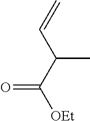 | ¹H NMR (400 MHz, Methanol-d4) δ 7.28-7.08 (m, 3H), 6.92 (t, J = 1.5 Hz, 1H), 6.54 (d, J = 1.5 Hz, 2H), 3.24-3.07 (m, 2H), 2.90- 2.74 (m, 2H), 2.33 (d, J = 2.0 Hz, 3H), 1.22 (td, J = 7.3, 1.2 Hz, 6H), 1.17 (d, J = 6.3 Hz, 3H). LCMS m/z 370.3 [M + H]⁺ |
| 103 | From S5^{1,3,7} 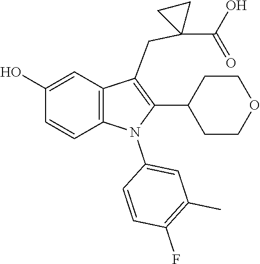 [SMILES: Cc1cc(-n2c(C3CCOCC3)c(CC3(C(=O)O)CC3)c3cc(O)ccc32)ccc1F] | 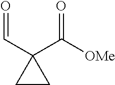 | ¹H NMR (400 MHz, Methanol-d4) δ 7.25-7.15 (m, 2H), 7.13 (ddd, J = 8.9, 4.6, 2.7 Hz, 1H), 6.89 (dd, J = 1.9, 1.0 Hz, 1H), 6.61-6.47 (m, 2H), 3.93-3.81 (m, 2H), 3.47 (s, 2H), 3.39-3.31 (m, 5H), 3.10-2.96 (m, 1H), 2.34 (d, J = 1.9 Hz, 3H), 2.04- 1.82 (m, 2H), 1.62 (d, J = 13.5 Hz, 3H), 1.28 (s, 2H), 0.92 (q, J = 3.3 Hz, 2H), 0.45 (q, J = 3.4 Hz, 2H). LCMS m/z 424.4 [M + H]⁺ |
| 104 | From S8^{1,3,4} 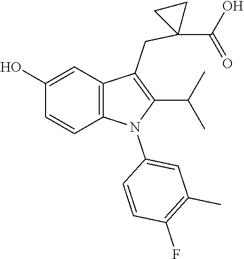 [SMILES: Cc1cc(-n2c(C(C)C)c(CC3(C(=O)O)CC3)c3cc(O)ccc32)ccc1F] | 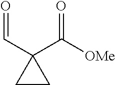 | ¹H NMR (400 MHz, Chloroform-d) δ 7.11 (dq, J = 9.2, 2.8, 2.4 Hz, 3H), 6.94 (dd, J = 2.3, 0.7 Hz, 1H), 6.75- 6.47 (m, 2H), 3.47 (s, 2H), 3.09 (p, J = 7.2 Hz, 1H), 2.33 (d, J = 2.0 Hz, 3H), 2.18 (s, 1H), 1.23 (d, J = 3.0 Hz, 2H), 1.20 (d, J = 2.9 Hz, 6H), 0.83 (q, J = 4.0 Hz, 2H). LCMS m/z 381.0 [M + H]⁺ |

1. Reductive alkylation: Et3SiH, TFA, CH₂Cl₂ at 50° C.
2. Hydrogenation: H₂, Pd(OH)₂
3. Hydrolysis conditions: LiOH, THF, MeOH, H₂O
4. Hydrogenation: H₂, Pd/C, MeOH or EtOAc
5. Hydrolysis conditions: NaOH, MeOH
6. SFC chiral separation to obtain individual stereoisomer.
7. Hydrogenation: H₂, Pd/C on wood, EtOAc
8. BBr₃, CH₂Cl₂
9. Reductive alkylation: Et₃SiH, MeSO₃H, CH₂Cl₂ at 50° C.
10. Hydrolysis conditions: KOH, MeOH, THF, H₂O, 70° C.
11. Note: N-monofluorophenyl substitution obtained from overreduction of N-3-chloro-4-fluorophenyl intermediate under hydrogenation conditions
12. Final compound is a racemic mixture of isomers
13. Reduction conditions: Mg, MeOH
14. Final compound is single stereoisomer of unknown absolute configuration
15. Final compound is a mixture of cis and trans isomers

### Compound 105

### Synthesis of cis-2-(3-(1-(4-fluoro-3-methylphenyl)-5-hydroxy-2-(tetrahydro-2H-pyran-4-yl)-1H-indol-3-yl)cyclohexyl)acetic acid (105)

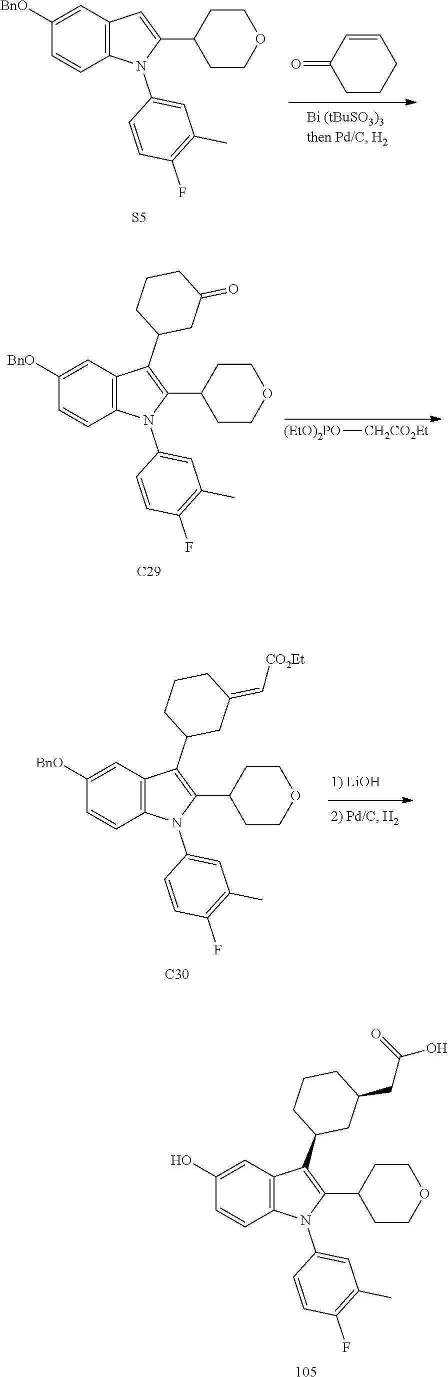

### Step 1. Synthesis of 3-(5-(benzyloxy)-1-(4-fluoro-3-methylphenyl)-2-(tetrahydro-2H-pyran-4-yl)-1H-indol-3-yl)cyclohexan-1-one (C29)

To a suspension of 5-benzyloxy-1-(4-fluoro-3-methyl-phenyl)-2-tetrahydropyran-4-yl-indole S5 (0.30 g, 0.69 mmol) and cyclohex-2-en-1-one (0.10 mL, 1.04 mmol) in CH₃CN (6 mL) was added bismuth; 2-methylpropane-2-sulfonate (0.06 g, 0.10 mmol). The suspension was stirred at room temperature for 1 hour. The solvent was removed under reduced pressure, the crude product was dissolved in EtOAc (10 mL) and washed with water. The organic phase was dried (MgSO₄), filtered, and concentrated in vacuo. The resulting residue was purified by silica gel chromatography (40 g ISCO column) using 0-40% EtOAc/heptanes gradient to afford 300 mg of product. 3-[5-benzyloxy-1-(4-fluoro-3-methyl-phenyl)-2-tetrahydropyran-4-yl-indol-3-yl]cyclo-hexanone (83%). ¹H NMR (400 MHz, DMSO-d6) δ 8.74 (s, 1H), 7.43-7.30 (m, 2H), 7.26-7.05 (m, 2H), 6.62 (d, J=8 Hz, 1H), 6.56 (dd, J=8.8, 2.1 Hz, 1H), 3.85 (d, J=11.0 Hz, 2H), 3.42 (d, J=12.9 Hz, 1H), 3.22-3.05 (m, 3H), 2.80-2.63 (m, 2H), 2.42-2.24 (m, 1H), 2.21-2.11 (m, 1H), 1.92-1.75 (m, 3H), 1.65-1.62 (m, 2H). ESI-MS m/z calc. 511.25, found 512.6 (M+1)⁺. To a mixture of 3-[5-benzyloxy-1-(4-fluoro-3-methyl-phenyl)-2-tetrahydropyran-4-yl-indol-3-yl] cyclohexanone (0.07 g) in MeOH (5 mL) and EtOAc (2 mL) was added Pd on C, wet, Degussa (0.05 g, 0.05 mmol). The suspension was purged with nitrogen. The system was evacuated and purged with hydrogen and then the mixture was stirred under an atmosphere of hydrogen for 3 hours. The mixture was filtered, and filtrate was concentrated under reduced pressure. The resulting residue was purified by silica gel chromatography (40 g ISCO column) using 0-60% EtOAc/heptanes gradient to afford 60 mg of product. 3-[1-(4-fluoro-3-methyl-phenyl)-5-hydroxy-2-tetrahydropyran-4-yl-indol-3-yl]cyclohexanone (20%). ¹H NMR (400 MHz, DMSO-d6) δ 8.74 (s, 1H), 7.43-7.30 (m, 2H), 7.26-7.05 (m, 2H), 6.62 (d, J=8 Hz, 1H), 6.56 (dd, J=8.8, 2.1 Hz, 1H), 3.85 (d, J=11.0 Hz, 2H), 3.42 (d, J=12.9 Hz, 1H), 3.22-3.05 (m, 3H), 2.80-2.63 (m, 2H), 2.42-2.24 (m, 1H), 2.21-2.11 (m, 1H), 1.92-1.75 (m, 3H), 1.65-1.62 (m, 2H). ESI-MS m/z calc. 421.21, found 422.59 (M+1)⁺.

### Step 2. Synthesis of ethyl (E)-2-(3-(5-(benzyloxy)-1-(4-fluoro-3-methylphenyl)-2-(tetrahydro-2H-pyran-4-yl)-1H-indol-3-yl)cyclohexylidene)acetate (C30)

To a solution of ethyl 2-diethoxyphosphorylacetate (0.26 g, 1.15 mmol) in THE (5 mL) was added KOtBu (0.13 g, 1.16 mmol). The reaction mixture was stirred at room temperature for 30 minutes. A solution of 3-[5-benzyloxy-1-(4-fluoro-3-methyl-phenyl)-2-tetrahydropyran-4-yl-indol-3-yl]cyclohexanone C29 (0.30 g, 0.57 mmol) in THE (5 mL) was added dropwise. The reaction mixture was stirred at room temperature for 3 hours. The solvent was evaporated under reduced pressure and the residue was dissolved in water (10 mL). The aqueous phase was extracted twice with EtOAc and the combined organic phases were dried over Na₂SO₄, filtered and concentrated in vacuo. The resulting residue was purified by silica gel chromatography (40 g ISCO column) using 0-40% EtOAc/heptanes gradient to afford 240 mg of product. Ethyl-2-[3-[5-benzyloxy-1-(4-fluoro-3-methyl-phenyl)-2-tetrahydropyran-4-yl-indol-3-yl]cyclohexylidene]acetate (69%). ESI-MS m/z calc. 581.29, found 582.57 (M+1)⁺.

### Step 3. cis-2-(3-(1-(4-fluoro-3-methylphenyl)-5-hydroxy-2-(tetrahydro-2H-pyran-4-yl)-1H-indol-3-yl)cyclohexyl)acetic acid (105)

To a solution of ethyl (2E)-2-[3-[5-benzyloxy-1-(4-fluoro-3-methyl-phenyl)-2-tetrahydropyran-4-yl-indol-3-yl]cyclohexylidene]acetate C30 (0.18 g, 0.30 mmol) in MeOH (5 mL), THE (1 mL) and water (1 mL) was added LiOH. The reaction mixture was stirred at room temperature for 18 hours. The solvent was removed under reduced pressure. The crude product was dissolved in water (5 mL) and acidified with 6N HCl. The aqueous phase was acidified with 6M HCl. The aqueous phase was extracted three times with EtOAc. The combined organic phases were dried over Na₂SO₄, filtered and concentrated in vacuo to afford 160 mg of product. 2-[3-[5-benzyloxy-1-(4-fluoro-3-methyl-phenyl)-2-tetrahydropyran-4-yl-indol-3-yl]cyclohexylidene]acetic acid (96%). ESI-MS m/z calc. 553.26, found 554.49 (M+1)⁺. The product (155 mg) was dissolved in methanol (5 mL) and Pd on C, wet, Degussa (0.10 g, 0.09 mmol) was added. The system was evacuated and purged with hydrogen and the mixture was stirred under an atmosphere of hydrogen for 3 hours. The solution was filtered and the filtrate was concentrated under reduced pressure. The resulting residue was purified by silica gel chromatography (40 g ISCO column) using 0-80% EtOAc/heptanes gradient to afford 112 mg of product. Racemic cis-2-[3-[1-(4-fluoro-3-methyl-phenyl)-5-hydroxy-2-tetrahydropyran-4-yl-indol-3-yl]cyclohexyl]acetic acid (78%). ¹H NMR (400 MHz, chloroform-d) δ 7.25-7.00 (m, 4H), 6.61 (d, J=8.7 Hz, 1H), 6.54 (dd, J=8.8, 2.2 Hz, 1H), 3.96 (d, J=11.5 Hz, 2H), 3.29-3.27 (m, 2H), 3.16-3.05 (m, 1H), 2.81-2.75 (m, 1H), 2.73-2.47 (m, 1H), 2.34 (s, 3H), 2.27 (q, J=6.7, 5.8 Hz, 1H), 2.17-1.45 (m, 12H), 1.24-1.08 (m, 1H). ESI-MS m/z calc. 465.2, found 466.6 (M+1)⁺.

### Compounds 106 and 107

### Synthesis of trans-3-(1-(4-fluoro-3-methylphenyl)-5-hydroxy-2-(tetrahydro-2H-pyran-4-yl)-1H-indol-3-yl)-1-(methoxymethyl)cyclobutane-1-carboxylic acid (106) and cis-3-(1-(4-fluoro-3-methylphenyl)-5-hydroxy-2-(tetrahydro-2H-pyran-4-yl)-1H-indol-3-yl)-1-(methoxymethyl)cyclobutane-1-carboxylic acid (107)

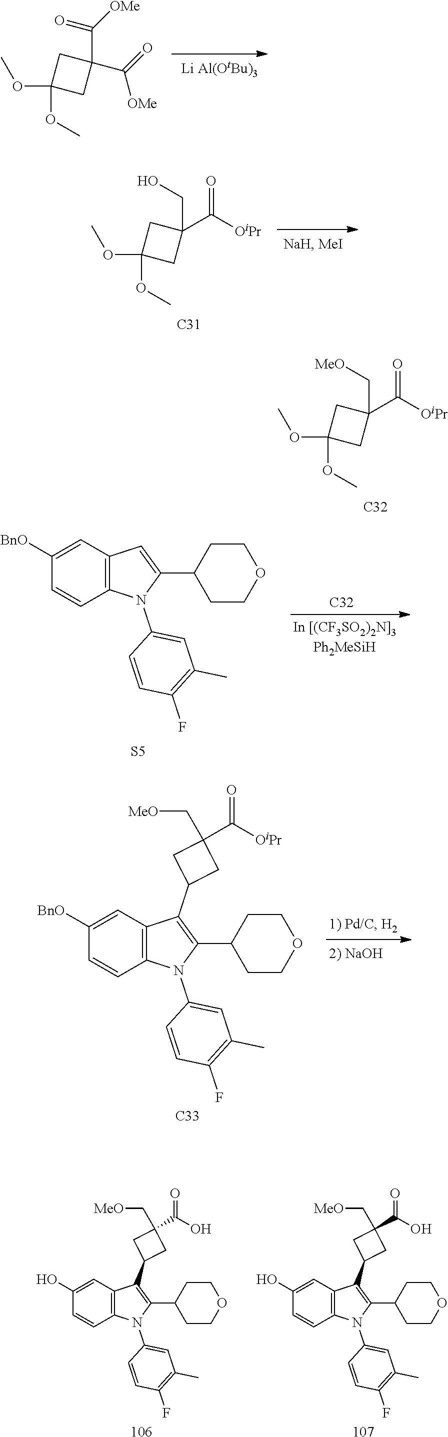

### Step 1. Synthesis of isopropyl 1-(hydroxymethyl)-3,3-dimethoxycyclobutane-1-carboxylate (C31)

To a cold (−78° C.) solution of diisopropyl 3,3-dimethoxycyclobutane-1,1-dicarboxylate (10.00 g, 34.68 mmol) in THE (40 mL) was added lithium tritert-butylaluminum hydride (80.0 mL of 1 M solution, 80.0 mmol). The mixture was stirred overnight at room temperature and then heated to 50° C. for 2 hours. The mixture was cooled to room temperature and quenched with aqueous saturated NH₄Cl solution. The mixture was extracted with CH₂Cl₂. The organic phase was dried over Na₂SO₄, filtered and concentrated in vacuo. The resulting residue was purified by silica gel chromatography (40 g ISCO column) using 0-40% EtOAc/heptanes gradient to afford 4.5 g of product. Isopropyl 1-(hydroxymethyl)-3,3-dimethoxy-cyclobutanecarboxylate (56%). ¹H NMR (400 MHz, Chloroform-d) δ 5.08 (p, J=6.3 Hz, 1H), 3.83 (d, J=6.6 Hz, 2H), 3.25-3.11 (m, 6H), 2.61-2.48 (m, 2H), 2.42 (td, J=6.5, 1.1 Hz, 1H), 2.26-2.14 (m, 2H), 1.35-1.22 (m, 6H).

### Step 2. Synthesis of isopropyl 3,3-dimethoxy-1-(methoxymethyl)cyclobutane-1-carboxylate (C32)

To a solution of isopropyl 1-(hydroxymethyl)-3,3-dimethoxy-cyclobutanecarboxylate C31 (1.00 g, 4.31 mmol) in DMF (10 mL) was added NaH (0.27 g of 60% w/w, 6.67 mmol). The reaction mixture was stirred for 10 minutes. To the mixture was added methyliodide (4.00 mL of 2 M solution, 8.00 mmol). The reaction was stirred for 2 hours at room temperature. The reaction was quenched with aqueous saturated NH₄Cl solution. The aqueous phase was extracted with EtOAc. The organic phase was washed with brine, dried over Na₂SO₄, filtered and concentrated in vacuo. The resulting residue was purified by silica gel chromatography (40 g ISCO column) using 0-20% EtOAc/heptanes gradient to afford 180 mg of product. Isopropyl 3,3-dimethoxy-1-(methoxymethyl)cyclobutanecarboxylate (17%). ¹H NMR (400 MHz, Chloroform-d) δ 5.07 (hept, J=6.2 Hz, 1H), 3.63 (s, 2H), 3.36 (s, 3H), 3.16 (d, J=2.2 Hz, 6H), 2.62-2.51 (m, 2H), 2.25-2.10 (m, 2H), 1.26 (d, J=6.3 Hz, 6H).

### Step 3. Synthesis of isopropyl 3-(5-(benzyloxy)-1-(4-fluoro-3-methylphenyl)-2-(tetrahydro-2H-pyran-4-yl)-1H-indol-3-yl)-1-(methoxymethyl)cyclobutane-1-carboxylate (C33)

To a vial charged with bis(trifluoromethylsulfonyl)azanide; indium(3+) (0.045 g, 0.047 mmol) was added dioxane (0.5 mL) and mixture was stirred for 5 minutes. To the mixture was added 5-benzyloxy-1-(4-fluoro-3-methyl-phenyl)-2-tetrahydropyran-4-yl-indole S5 (0.200 g, 0.481 mmol), isopropyl 3,3-dimethoxy-1-(methoxymethyl)cyclobutanecarboxylate C₃₂ (0.130 g, 0.528 mmol) and methyl(diphenyl)silane (0.120 g, 0.605 mmol). The reaction mixture was heated at 47° C. for 90 min and then concentrated in vacuo. The resulting residue was purified by silica gel chromatography (40 g ISCO column) using 0-30% CH₂Cl₂/heptanes gradient to afford 170 mg of product. ESI-MS m/z calc. 599.3, found 600.0 (M+1)⁺.

### Step 4. Synthesis of trans-3-(1-(4-fluoro-3-methylphenyl)-5-hydroxy-2-(tetrahydro-2H-pyran-4-yl)-1H-indol-3-yl)-1-(methoxymethyl)cyclobutane-1-carboxylic acid (106) and cis-3-(1-(4-fluoro-3-methylphenyl)-5-hydroxy-2-(tetrahydro-2H-pyran-4-yl)-1H-indol-3-yl)-1-(methoxymethyl)cyclobutane-1-carboxylic acid (107)

To a solution of isopropyl 3-[5-benzyloxy-1-(4-fluoro-3-methyl-phenyl)-2-tetrahydropyran-4-yl-indol-3-yl]-1-(methoxymethyl)cyclobutanecarboxylate C33 (0.169 g, 0.283 mmol) in MeOH (10 mL) was added Pd/C (0.050 g of 10% w/w, 0.047 mmol). The mixture was stirred under an atmosphere of hydrogen for 1 hour. The mixture was filtered though a pad of celite and filtrate concentrated in vacuo to afford 100 mg of 3-[1-(4-fluoro-3-methyl-phenyl)-5-hydroxy-2-tetrahydropyran-4-yl-indol-3-yl]-1-(methoxymethyl)cyclobutanecarboxylate (69%). ESI-MS m/z calc. 509.26, found 510.0 (M+1)⁺.

To a solution of the product in MeOH (10 mL) was added NaOH (0.50 mL of 3 M solution, 1.50 mmol). The mixture was stirred at 50° C. for 1 hour. The reaction was neutralized with 1 N HCl and extracted with CH₂Cl₂. The resulting residue was purified by reverse phase HPLC to afford 10.4 mg of product. Trans-3-[1-(4-fluoro-3-methyl-phenyl)-5-hydroxy-2-tetrahydropyran-4-yl-indol-3-yl]-1-(methoxymethyl)cyclobutanecarboxylic acid (7%). ¹H NMR (400 MHz, Methanol-d4) δ 7.44 (dd, J=2.1, 0.8 Hz, 1H), 7.28-7.15 (m, 2H), 7.13-7.03 (m, 1H), 6.68-6.48 (m, 2H), 4.11 (p, J=9.8 Hz, 1H), 4.01-3.87 (m, 2H), 3.80 (s, 2H), 2.84 (q, J=13.8, 12.5 Hz, 3H), 2.71-2.58 (m, 2H), 2.34 (d, J=2.0 Hz, 3H), 2.03 (q, J=12.7 Hz, 2H), 1.63 (d, J=13.4 Hz, 2H). ESI-MS m/z calc. 467.2, found 468.5 (M+1)⁺. Cis-3-[1-(4-fluoro-3-methyl-phenyl)-5-hydroxy-2-tetrahydropyran-4-yl-indol-3-yl]-1-(methoxymethyl)cyclobutanecarboxylic acid (10.9 mg, 7%). ¹H NMR (400 MHz, Methanol-d4) δ 7.28-7.18 (m, 3H), 7.18-7.08 (m, 1H), 4.13 (tt, J=10.2, 9.0 Hz, 1H), 3.92 (dt, J=11.2, 3.1 Hz, 3H), 3.71 (s, 2H), 3.40 (s, 4H), 3.36 (dd, J=11.0, 3.7 Hz, 1H), 2.94-2.77 (m, 4H), 2.75-2.62 (m, 3H), 2.34 (d, J=2.3 Hz, 3H), 1.77 (td, J=9.9, 8.9, 3.8 Hz, 4H). ESI-MS m/z calc. 467.21, found 468.58 (M+1)⁺.

### Preparation of C₃₄

### isopropyl 1-(fluoromethyl)-3,3-dimethoxycyclobutane-1-carboxylate (C34)

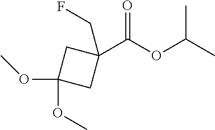 [SMILES: COC1(OC)CC(CF)(C(=O)OC(C)C)C1]

To a cold (−78° C.) solution of isopropyl 1-(hydroxymethyl)-3,3-dimethoxy-cyclobutanecarboxylate C31 (1.37 g, 5.89 mmol) in CH₂Cl₂ (10 mL) was added 2,6-lutidine (1.00 mL, 8.63 mmol) and trifluoromethanesulfonic anhydride (1.20 mL, 7.13 mmol). The reaction mixture was stirred at −78° C. and gradually warmed to room temperature. The reaction was quenched with water and extracted with CH₂Cl₂. The organic phase was washed with aqueous saturated NaHCO₃ solution, aqueous saturated NH₄Cl solution and brine. The organic phase was dried over Na₂SO₄, filtered and concentrated in vacuo to afford 1.8 g of product.

Trifluoromethylsulfonyloxymethyl)cyclobutanecarboxylate. ¹H NMR (400 MHz, Chloroform-d) δ 5.10 (p, J=6.3 Hz, 1H), 4.81 (s, 2H), 3.18 (d, J=1.8 Hz, 6H), 2.65-2.55 (m, 2H), 2.29-2.20 (m, 2H), 1.28 (s, 6H). The product was dissolved in THE (10 mL) and cooled to −78° C. To the solution was added tetrabutylammonium fluoride (9.8 mL of 1 M solution in THF, 9.8 mmol). The reaction mixture was stirred at room temperature for 1 hour, quenched with water and extracted with EtOAc. The organic phase was washed with brine, dried (MgSO₄), filtered, and concentrated in vacuo. The resulting residue was purified by silica gel chromatography (40 g ISCO column) using 0-30% EtOAc/heptanes gradient to afford 0.8 g of product. Isopropyl 1-(fluoromethyl)-3,3-dimethoxy-cyclobutanecarboxylate (58%). ¹H NMR (400 MHz, Chloroform-d) δ 5.08 (p, J=6.3 Hz, 1H), 4.71 (s, 1H), 4.59 (s, 1H), 3.17 (d, J=0.6 Hz, 6H), 2.62-2.53 (m, 2H), 2.28-2.18 (m, 2H), 1.28 (d, J=6.3 Hz, 6H).

### Preparation C35

### isopropyl 3,3-dimethoxy-1-(methoxymethyl)cyclobutane-1-carboxylate (C35)

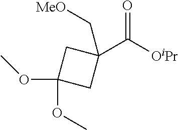 [SMILES: COCC1(C(=O)OC(C)C)CC(OC)(OC)C1]

To a solution of isopropyl 1-(hydroxymethyl)-3,3-dimethoxy-cyclobutanecarboxylate C₃₁ (1.00 g, 4.31 mmol) in DMF (10 mL) was added NaH (0.27 g of 60% w/w, 6.67 mmol) and the mixture was stirred for 10 minutes. To the mixture was added Mel (4.00 mL of 2 M, 8.00 mmol). The reaction mixture was stirred at room temperature for 2 hours. The reaction was quenched by addition of aqueous saturated NH₄Cl solution and extracted with EtOAc. The organic phase washed with brine, dried over Na₂SO₄, filtered and concentrated in vacuo. The resulting residue was purified by silica gel chromatography (40 g ISCO column) using 0-20% EtOAc/heptanes gradient to afford 180 mg of product. Isopropyl 3,3-dimethoxy-1-(methoxymethyl)cyclobutanecarboxylate (17%). ¹H NMR (400 MHz, Chloroform-d) δ 5.07 (hept, J=6.2 Hz, 1H), 3.63 (s, 2H), 3.36 (s, 3H), 3.16 (d, J=2.2 Hz, 6H), 2.62-2.51 (m, 2H), 2.25-2.10 (m, 2H), 1.26 (d, J=6.3 Hz, 6H).

### Compounds 108-122

Compounds 108-122 were prepared using a method described for the preparation of compound 106 and 107 using the appropriate ketal or ketone and the relevant indole intermediate. Any modifications to this method are noted in the table footnotes.

TABLE 8
Method of preparation, structure and physicochemical data for compounds 108-122

| Compound | Method/Product | Ketal/Ketone | ¹H NMR; LCMS m/z [M + H]⁺ |
|---|---|---|---|
| 108 | 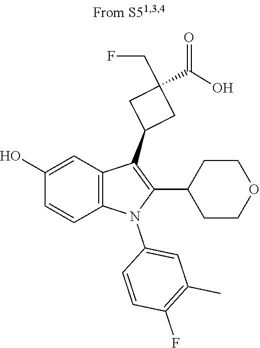 [SMILES: Cc1cc(-n2c(C3CCOCC3)c([C@H]3C[C@](CF)(C(=O)O)C3)c3cc(O)ccc32)ccc1F] | 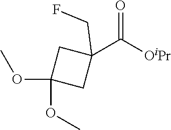 | ¹H NMR (400 MHz, Methanol- d4) δ 7.35 (d, J = 2.1 Hz, 1H), 7.28-7.14 (m, 3H), 7.14-7.05 (m, 1H), 6.66-6.55 (m, 2H), 4.83 (s, 1H), 4.71 (s, 1H), 4.21 (d, J = 9.6 Hz, 1H), 3.95 (dd, J = 11.5, 4.2 Hz, 3H), 2.98-2.77 (m, 2H), 2.71-2.57 (m, 2H), 2.34 (d, J = 2.1 Hz, 4H), 2.11- 1.97 (m, 2H), 1.64 (d, J = 13.5 Hz, 2H). LCMS m/z 456.5 [M + H]⁺. |
| 109 | 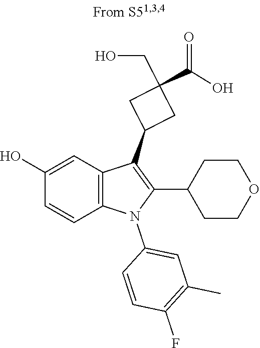 [SMILES: Cc1cc(-n2c(C3CCOCC3)c([C@H]3C[C@@](CO)(C(=O)O)C3)c3cc(O)ccc32)ccc1F] | 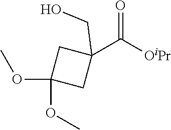 | ¹H NMR (400 MHz, Methanol- d4) δ 7.42 (d, J = 2.0 Hz, 1H), 7.26-7.14 (m, 2H), 7.07 (ddd, J = 8.4, 4.5, 2.7 Hz, 1H), 6.68- 6.52 (m, 2H), 4.23-4.03 (m, 1H), 3.94 (d, J = 17.2 Hz, 4H), 3.26 (dd, J = 11.9, 2.0 Hz, 2H), 2.82 (td, J = 11.0, 10.0, 3.3 Hz, 3H), 2.62 (td, J = 9.0, 2.6 Hz, 2H), 2.33 (d, J = 1.9 Hz, 3H), 2.11-1.91 (m, 2H), 1.62 (dd, J = 13.2, 3.8 Hz, 2H). LCMS m/z 454.3 [M + H]⁺. |
| 110 | 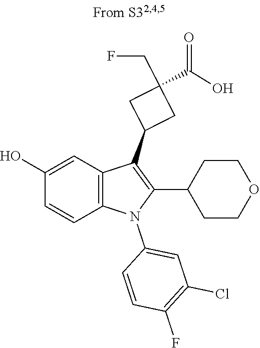 [SMILES: O=C(O)[C@]1(CF)C[C@@H](c2c(C3CCOCC3)n(-c3ccc(F)c(Cl)c3)c3ccc(O)cc32)C1] | 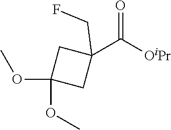 | ¹H NMR (400 MHz, Methanol- d4) δ 7.45 (dd, J = 6.6, 2.5 Hz, 1H), 7.37 (t, J = 8.8 Hz, 1H), 7.25 (ddd, J = 6.2, 4.3, 2.1 Hz, 1H), 6.73 (s, 1H), 6.56-6.46 (m, 2H), 4.11 (p, J = 9.5 Hz, 1H), 3.84 (d, J = 11.3 Hz, 2H), 3.42-3.24 (m, 3H), 3.16-3.00 (m, 2H), 2.74 (td, J = 10.8, 4.7 Hz, 1H), 2.31 (td, J = 9.4, 2.6 Hz, 2H), 1.80-1.58 (m, 4H). LCMS m/z 476.5 [M + H]⁺. |
| 111 | 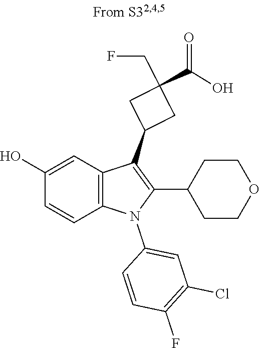 [SMILES: O=C(O)[C@]1(CF)C[C@H](c2c(C3CCOCC3)n(-c3ccc(F)c(Cl)c3)c3ccc(O)cc32)C1] | 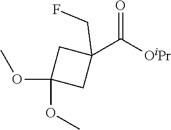 | ¹H NMR (400 MHz, Methanol- d4) δ 7.43 (dd, J = 6.6, 2.5 Hz, 1H), 7.41-7.30 (m, 2H), 7.24 (ddd, J = 8.7, 4.3, 2.6 Hz, 1H), 6.56-6.48 (m, 2H), 6.41 (s, 1H), 4.15-4.02 (m, 1H), 3.84 (d, J = 11.5 Hz, 2H), 3.35-3.24 (m, 2H), 2.82 (td, J = 10.0, 2.5 Hz, 2H), 2.73 (dq, J = 10.4, 5.4 Hz, 1H), 2.62 (td, J = 9.5, 2.6 Hz, 2H), 1.74-1.62 (m, 4H). LCMS m/z 476.5 [M + H]⁺. |
| 112 | 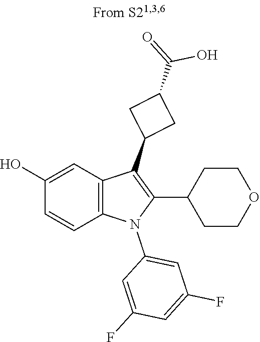 [SMILES: O=C(O)[C@H]1C[C@H](c2c(C3CCOCC3)n(-c3cc(F)cc(F)c3)c3ccc(O)cc32)C1] | 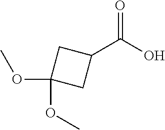 | ¹H NMR (400 MHz, Methanol- d4) δ 7.21-7.10 (m, 1H), 7.06 (dd, J = 7.6, 2.2 Hz, 2H), 6.70 (d, J = 8.7 Hz, 1H), 6.62 (d, J = 8.7 Hz, 1H), 6.52 (s, 1H), 4.27 (s, 0H), 3.94 (dt, J = 11.3, 3.2 Hz, 2H), 3.39 (dd, J = 14.6, 11.3 Hz, 1H), 3.22 (d, J = 0.9 Hz, 1H), 2.99 (s, 1H), 2.64 (s, 1H), 1.77 (dd, J = 8.9, 3.5 Hz, 4H). LCMS m/z 428.3 [M + H]⁺. |
| 113 | 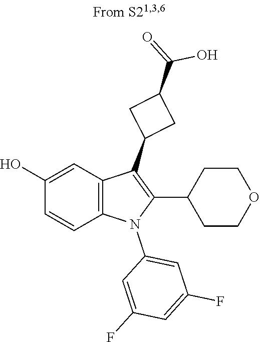 [SMILES: O=C(O)[C@H]1C[C@@H](c2c(C3CCOCC3)n(-c3cc(F)cc(F)c3)c3ccc(O)cc32)C1] | 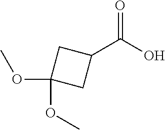 | ¹H NMR (400 MHz, Methanol- d4) δ 7.20-7.03 (m, 3H), 6.81 (t, J = 0.7 Hz, 1H), 6.70 (dd, J = 8.7, 0.7 Hz, 1H), 6.60 (d, J = 8.7 Hz, 1H), 4.03 (s, 0H), 3.97- 3.90 (m, 2H), 3.40 (td, J = 11.4, 3.1 Hz, 2H), 3.22-3.06 (m, 1H), 3.00-2.86 (m, 3H), 2.61 (dd, J = 8.7, 2.8 Hz, 2H), 1.87- 1.72 (m, 3H). LCMS m/z 428.3 [M + H]⁺. |
| 114 | 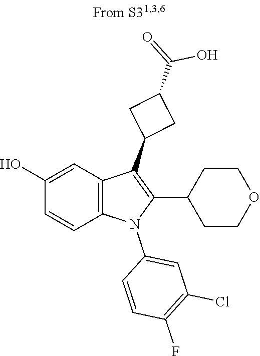 [SMILES: O=C(O)[C@H]1C[C@H](c2c(C3CCOCC3)n(-c3ccc(F)c(Cl)c3)c3ccc(O)cc32)C1] | 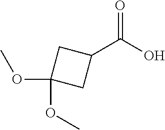 | ¹H NMR (400 MHz, Methanol- d4) δ 7.50 (d, J = 2.0 Hz, 1H), 7.30 (d, J = 6.6 Hz, 4H), 6.70- 6.50 (m, 2H), 4.05-3.86 (m, 3H), 2.98 (d, J = 10.4 Hz, 2H), 2.84 (s, 1H), 2.54 (d, J = 9.3 Hz, 2H), 2.05 (dd, J = 13.0, 4.4 Hz, 2H), 1.70-1.52 (m, 2H) LCMS m/z 444.3 [M + H]⁺. |
| 115 | 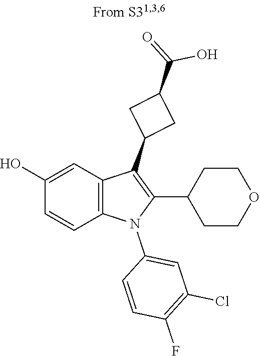 [SMILES: O=C(O)[C@H]1C[C@@H](c2c(C3CCOCC3)n(-c3ccc(F)c(Cl)c3)c3ccc(O)cc32)C1] | 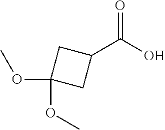 | ¹H NMR (400 MHz, Methanol- d4) δ 7.20-7.03 (m, 3H), 6.81 (t, J = 0.7 Hz, 1H), 6.70 (dd, J = 8.7, 0.7 Hz, 1H), 6.60 (d, J = 8.7 Hz, 1H), 4.03 (s, 0H), 3.97- 3.90 (m, 2H), 3.40 (td, J = 11.4, 3.1 Hz, 2H), 3.22-3.06 (m, 1H), 3.00-2.86 (m, 3H), 2.61 (dd, J = 8.7, 2.8 Hz, 2H), 1.87- 1.72 (m, 3H). LCMS m/z 428.3 [M + H]⁺. |
| 116 | 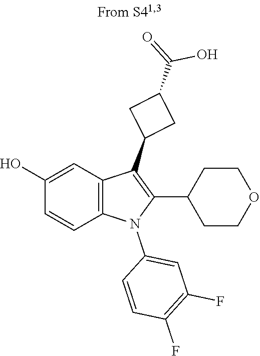 [SMILES: O=C(O)[C@H]1C[C@H](c2c(C3CCOCC3)n(-c3ccc(F)c(F)c3)c3ccc(O)cc32)C1] | 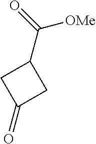 | ¹H NMR (400 MHz, Methanol- d4) δ 7.47 (q, J = 9.3 Hz, 1H), 7.40-7.21 (m, 2H), 7.12 (d, J = 9.0 Hz, 1H), 6.74-6.55 (m, 2H), 4.27 (t, J = 9.3 Hz, 1H), 3.95 (dd, J = 11.6, 4.3 Hz, 2H), 3.00 (q, J = 10.6, 10.1 Hz, 2H), 2.85-2.75 (m, 1H), 2.63 (ddd, J = 13.2, 9.8, 3.3 Hz, 3H), 2.17- 1.94 (m, 2H), 1.65 (d, J = 13.1 Hz, 2H). LCMS m/z 428.5 [M + H]⁺ |
| 117 | 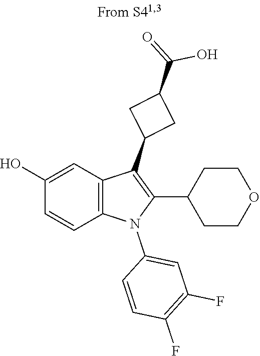 [SMILES: O=C(O)[C@H]1C[C@@H](c2c(C3CCOCC3)n(-c3ccc(F)c(F)c3)c3ccc(O)cc32)C1] | 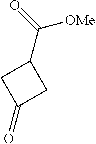 | ¹H NMR (400 MHz, Methanol- d4) δ 7.58-7.39 (m, 2H), 7.28 (ddd, J = 11.0, 7.1, 2.5 Hz, 1H), 7.18-7.07 (m, 1H), 6.73-6.54 (m, 2H), 4.04-3.87 (m, 3H), 3.27-3.13 (m, 1H), 2.98 (q, J = 10.2 Hz, 2H), 2.83 (ddt, J = 12.5, 7.8, 3.8 Hz, 1H), 2.54 (qd, J = 8.4, 2.6 Hz, 2H), 2.15-1.97 (m, 2H), 1.67 (d, J = 12.2 Hz, 2H). LCMS m/z 428.5 [M + H]⁺ |
| 118 | 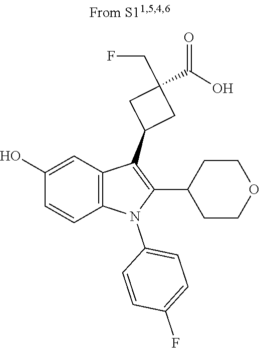 [SMILES: O=C(O)[C@]1(CF)C[C@@H](c2c(C3CCOCC3)n(-c3ccc(F)cc3)c3ccc(O)cc32)C1] | 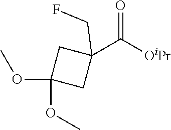 | ¹H NMR (400 MHz, Methanol- d4) δ 7.49-7.25 (m, 5H), 6.66- 6.53 (m, 2H), 4.76 (d, J = 47.7 Hz, 2H), 4.31-4.14 (m, 1H), 4.02-3.83 (m, 2H), 2.98-2.73 (m, 3H), 2.70-2.58 (m, 3H), 2.42 (d, J = 9.4 Hz, 0H), 2.15- 1.96 (m, 2H), 1.85-1.56 (m, 3H). LCMS m/z 442.5 [M + H]⁺ |
| 119 | 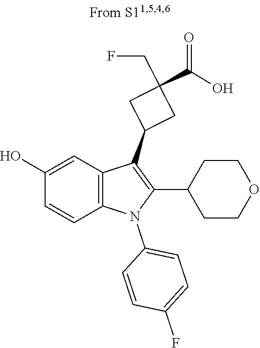 [SMILES: O=C(O)[C@]1(CF)C[C@H](c2c(C3CCOCC3)n(-c3ccc(F)cc3)c3ccc(O)cc32)C1] | 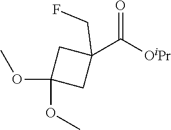 | ¹H NMR (400 MHz, Methanol- d4) δ 7.53 (d, J = 2.3 Hz, 1H), 7.36-7.25 (m, 3H), 6.67-6.50 (m, 2H), 5.49 (s, 1H), 4.09 (ddd, J = 9.3, 6.2, 3.2 Hz, 1H), 4.02-3.87 (m, 2H), 2.87-2.56 (m, 1H),3.33-3.14 (m, 2H), 2.38 (m, 2H), 1.70-1.47 (m, 4H). LCMS m/z 442.0 [M + H]⁺ |
| 120 | 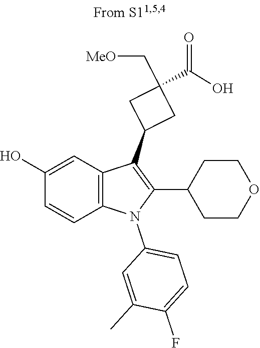 [SMILES: COC[C@]1(C(=O)O)C[C@H](c2c(C3CCOCC3)n(-c3ccc(F)c(C)c3)c3ccc(O)cc32)C1] | 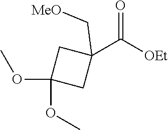 | ¹H NMR (400 MHz, Methanol- d4) δ 7.44 (dd, J = 2.1, 0.8 Hz, 1H), 7.28-7.15 (m, 2H), 7.13- 7.03 (m, 1H), 6.68-6.48 (m, 2H), 4.11 (p, J = 9.8 Hz, 1H), 4.01-3.87 (m, 2H), 3.80 (s, 2H), 2.84 (q, J = 13.8, 12.5 Hz, 3H), 2.71-2.58 (m, 2H), 2.34 (d, J = 2.0 Hz, 3H), 2.03 (q, J = 12.7 Hz, 2H), 1.63 (d, J = 13.4 Hz, 2H). LCMS m/z 468.5 [M + H]⁺ |
| 121 | 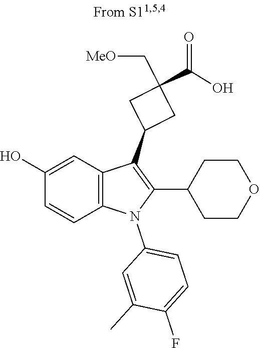 [SMILES: COC[C@]1(C(=O)O)C[C@@H](c2c(C3CCOCC3)n(-c3ccc(F)c(C)c3)c3ccc(O)cc32)C1] | 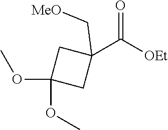 | ¹H NMR (400 MHz, Methanol- d4) δ 7.28-7.18 (m, 3H), 7.18- 7.08 (m, 1H), 4.13 (tt, J = 10.2, 9.0 Hz, 1H), 3.92 (dt, J = 11.2, 3.1 Hz, 3H), 3.71 (s, 2H), 3.40 (s, 4H), 3.36 (dd, J = 11.0, 3.7 Hz, 1H), 2.94-2.77 (m, 4H), 2.75-2.62 (m, 3H), 2.34 (d, J = 2.3 Hz, 3H), 1.77 (td, J = 9.9, 8.9, 3.8 Hz, 4H). LCMS m/z 468.6 [M + H]⁺ |
| 122 | 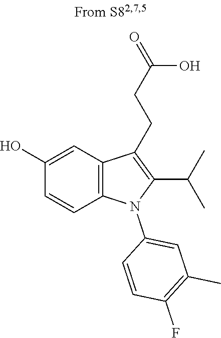 [SMILES: Cc1cc(-n2c(C(C)C)c(CCC(=O)O)c3cc(O)ccc32)ccc1F] | 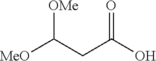 | ¹H NMR (400 MHz, Chloroform-d) δ 7.20-7.08 (m, 3H), 7.01 (d, J = 2.3 Hz, 1H), 6.75 (d, J = 8.7 Hz, 1H), 6.67 (dd, J = 8.7, 2.3 Hz, 1H), 3.27- 3.15 (m, 2H), 3.03 (p, J = 7.2 Hz, 1H), 2.81-2.71 (m, 2H), 2.36 (d, J = 2.0 Hz, 3H), 1.31 (d, J = 2.8 Hz, 3H), 1.29 (d, J = 2.8 Hz, 3H). LCMS m/z 355.4 [M + H]⁺ |

¹Reductive alkylation: In[CF₃SO₂)₂N]₃, Ph₂MeSiH, dioxane, 50° C.
²Reductive alkylation: Et₃SiH, TFA, CH₂Cl₂, 50° C.
³Hydrogenation: H₂, Pd(OH)₂
⁴Hydrolysis conditions: NaOH, MeOH
⁵Hydrogenation: H₂, Pd/C, MeOH
⁶SFC chiral chromatography
⁷Hydrolysis conditions: LiOH, MeOH, THF, H₂O

### Compound 123

### Synthesis of cis-1-(difluoromethyl)-3-(1-(4-fluoro-3-methylphenyl)-5-hydroxy-2-(tetrahydro-2H-pyran-4-yl)-1H-indol-3-yl)cyclobutane-1-carboxylic acid (123)

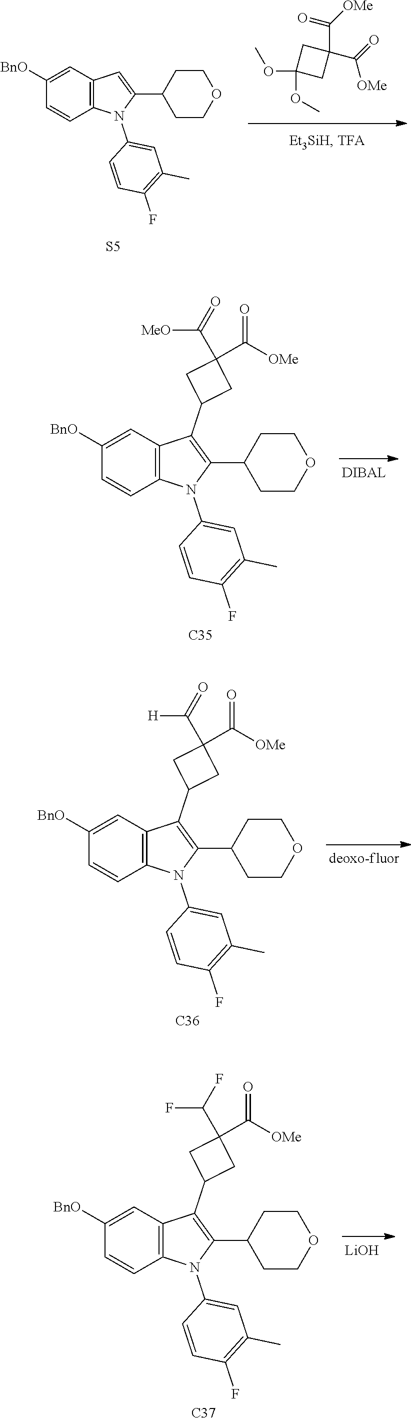

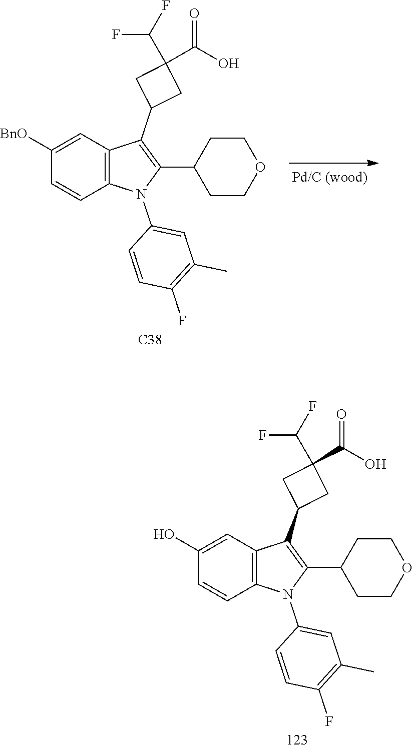

### Step 1. Synthesis of dimethyl 3-(5-(benzyloxy)-1-(4-fluoro-3-methylphenyl)-2-(tetrahydro-2H-pyran-4-yl)-1H-indol-3-yl)cyclobutane-1,1-dicarboxylate (C35)

To a solution of 5-benzyloxy-1-(4-fluoro-3-methyl-phenyl)-2-tetrahydropyran-4-yl-indole S5 (0.500 g, 1.203 mmol) and dimethyl 3-oxocyclobutane-1,1-dicarboxylate (0.500 g, 2.686 mmol) in CH₂Cl₂ (7.0 mL) was added triethylsilane (0.600 mL, 3.757 mmol) followed by 2,2,2-trifluoroacetic acid (0.250 mL, 3.245 mmol). The mixture was stirred at room temperature for 48 h. The reaction mixture was diluted with 15 mL of CH₂Cl₂ and washed with aqueous saturated. NaHCO₃ and brine. The organic phase was dried (MgSO₄), filtered, and concentrated in vacuo. The resulting residue was purified by silica gel chromatography (40 g ISCO column) using 0-60% EtOAc/heptanes gradient to afford 210 mg of product. Dimethyl 3-[5-benzyloxy-1-(4-fluoro-3-methyl-phenyl)-2-tetrahydropyran-4-yl-indol-3-yl]cyclobutane-1,1-dicarboxylate (30%). ¹H NMR (400 MHz, Chloroform-d) δ 7.73 (d, J=2.3 Hz, 1H), 7.58-7.51 (m, 2H), 7.45-7.32 (m, 2H), 7.20-7.03 (m, 4H), 6.86 (dd, J=8.8, 2.3 Hz, 1H), 6.78 (d, J=8.9 Hz, 1H), 5.22 (s, 2H), 4.14-4.04 (m, 1H), 3.92 (s, 3H), 3.85 (s, 3H), 3.48-3.26 (m, 2H), 3.03-2.94 (m, 1H), 2.36 (d, J=2.0 Hz, 3H), 2.02 (dtd, J=17.4, 12.4, 4.8 Hz, 2H), 1.68-1.50 (m, 2H). ESI-MS m/z calc. 585.25, found 586.02 (M+1)⁺.

### Step 2. Synthesis of methyl 3-(5-(benzyloxy)-1-(4-fluoro-3-methylphenyl)-2-(tetrahydro-2H-pyran-4-yl)-1H-indol-3-yl)-1-formylcyclobutane-1-carboxylate (C36)

To a cold (−78° C.) solution of dimethyl 3-[5-benzyloxy-1-(4-fluoro-3-methyl-phenyl)-2-tetrahydropyran-4-yl-indol-3-yl]cyclobutane-1,1-dicarboxylate C₃₅ (0.100 g, 0.171 mmol) in CH₂Cl₂ (3.0 mL) was added diisobutyl aluminum hydride (0.340 mL of 1 M solution, 0.340 mmol). The mixture was stirred at −78° C. for 3 h. The reaction was quenched with aqueous saturated NH₄Cl solution and extracted three times with EtOAc. The organic phase was dried (MgSO₄), filtered, and concentrated in vacuo. The resulting residue was purified by silica gel chromatography (40 g ISCO column) using 0-70% EtOAc/heptanes gradient to afford 33 mg of product. Methyl 3-[5-benzyloxy-1-(4-fluoro-3-methyl-phenyl)-2-tetrahydropyran-4-yl-indol-3-yl]-1-formyl-cyclobutanecarboxylate (35%). ESI-MS m/z calc. 555.24, found 556.32 (M+1)⁺.

### Step 3. Synthesis of methyl 3-(5-(benzyloxy)-1-(4-fluoro-3-methylphenyl)-2-(tetrahydro-2H-pyran-4-yl)-1H-indol-3-yl)-1-(difluoromethyl)cyclobutane-1-carboxylate (C37)

To a cold (0° C.) solution of methyl 3-[5-benzyloxy-1-(4-fluoro-3-methyl-phenyl)-2-tetrahydropyran-4-yl-indol-3-yl]-1-formyl-cyclobutanecarboxylate C₃₆ (0.032 g, 0.058 mmol) in CH₂Cl₂ (2 mL) was added Deoxo-fluor (0.023 mL, 0.125 mmol) and the mixture was warmed to room temperature and allowed to stir at that temperature for 2 hours. The reaction was quenched with ice and extracted with CH₂Cl₂. The organic phase was dried (MgSO₄), filtered, and concentrated in vacuo. The resulting residue was purified by silica gel chromatography (4 g ISCO column) using 0-50% EtOAc/heptanes gradient to afford 8 mg of product. Methyl 3-[5-benzyloxy-1-(4-fluoro-3-methyl-phenyl)-2-tetrahydropyran-4-yl-indol-3-yl]-1-(difluoromethyl)-cyclobutanecarboxylate (24%). ESI-MS m/z calc. 577.244, found 578.38 (M+1)⁺.

### Step 4. Synthesis of 3-(5-(benzyloxy)-1-(4-fluoro-3-methylphenyl)-2-(tetrahydro-2H-pyran-4-yl)-1H-indol-3-yl)-1-(difluoromethyl)cyclobutane-1-carboxylic acid (C38)

To a solution of methyl 3-[5-benzyloxy-1-(4-fluoro-3-methyl-phenyl)-2-tetrahydropyran-4-yl-indol-3-yl]-1-(difluoromethyl)cyclobutanecarboxylate C₃₇ (0.023 g, 0.039 mmol) in MeOH (0.6 mL), THE (0.25 mL) and H₂O (0.12 mL) and the mixture was stirred at 25° C. for 18 h. The solvent was evaporated under reduced pressure and the white solid was dissolved in water (10 mL) and slowly acidified with HCl (0.43 mL of 2 M solution, 0.86 mmol). The aqueous layer was extracted three times with EtOAc, dried (MgSO₄), filtered, and concentrated in vacuo to afford 21 mg of product. 3-[5-benzyloxy-1-(4-fluoro-3-methyl-phenyl)-2-tetrahydropyran-4-yl-indol-3-yl]-1-(difluoro-methyl)cyclobutanecarboxylic acid (86%). ESI-MS m/z calc. 563.23, found 564.42 (M+1)⁺.

### Step 5. Synthesis of cis-1-(difluoromethyl)-3-(1-(4-fluoro-3-methylphenyl)-5-hydroxy-2-(tetrahydro-2H-pyran-4-yl)-1H-indol-3-yl)cyclobutane-1-carboxylic acid (123)

To a solution of 3-[5-benzyloxy-1-(4-fluoro-3-methyl-phenyl)-2-tetrahydropyran-4-yl-indol-3-yl]-1-(difluoromethyl)cyclobutanecarboxylic acid C38 (0.021 g, 0.037 mmol) in EtOAc (1.0 mL) purged with nitrogen was added Pd/wood carbon (0.010 g of 10% w/w, 0.004 mmol). The reaction mixture was evacuated and purged with hydrogen and stirred under a hydrogen atmosphere for 2 hours. The crude mixture was filtered through a pad of celite and the filtrate was concentrated in vacuo. The resulting residue was purified by silica gel chromatography (12 g ISCO column) using 0-20% EtOAc/CH₂Cl₂ gradient to afford 9.3 mg of product. 1-(difluoromethyl)-3-[1-(4-fluoro-3-methyl-phenyl)-5-hydroxy-2-tetrahydropyran-4-yl-indol-3-yl]cyclobutanecarboxylic acid (48%). ¹H NMR (400 MHz, Chloroform-d) δ 7.70 (s, 1H), 7.17-6.94 (m, 4H), 6.72 (s, 2H), 6.40 (t, J=56.4 Hz, 1H), 4.19-4.03 (m, 1H), 4.00 (dd, J=11.6, 4.2 Hz, 2H), 3.30 (t, J=11.3 Hz, 4H), 2.75 (dt, J=22.1, 12.2 Hz, 3H), 2.33 (d, J=1.9 Hz, 3H), 2.02 (d, J=23.2 Hz, 3H), 1.60 (d, J=13.2 Hz, 2H), 1.35-1.11 (m, 2H). ESI-MS m/z calc. 473.18, found 474.31 (M+1)⁺.

### Compound 124

### Synthesis of trans-2-(1-(4-fluoro-3-methylphenyl)-5-hydroxy-2-(tetrahydro-2H-pyran-4-yl)-1H-indol-3-yl)cyclopropane-1-carboxylic acid (124)

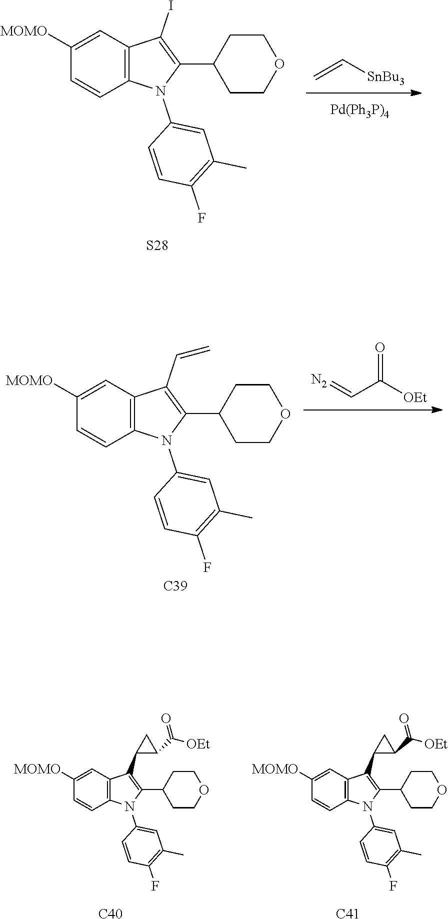

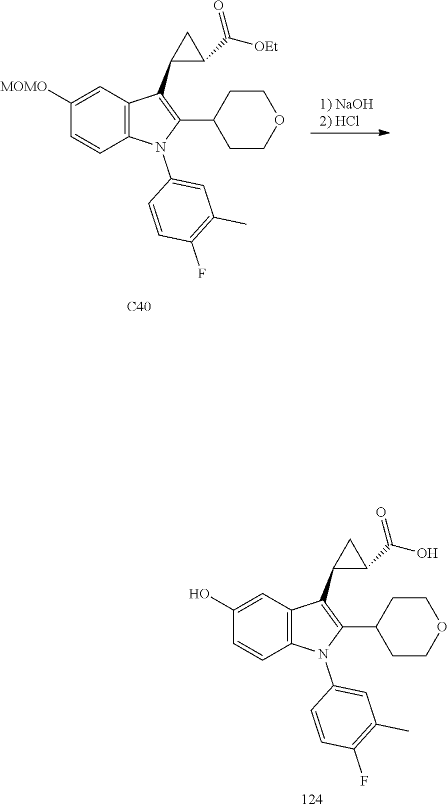

### Step 1. Synthesis of 1-(4-fluoro-3-methylphenyl)-5-(methoxymethoxy)-2-(tetrahydro-2H-pyran-4-yl)-3-vinyl-1H-indole (C39)

To a solution of 1-(4-fluoro-3-methyl-phenyl)-3-iodo-5-(methoxymethoxy)-2-tetrahydropyran-4-yl-indole S28 (0.23 g, 0.464 mmol), tetraethylammonium chloride (0.14 g, 0.85 mmol), palladium; triphenylphosphane (0.028 g, 0.024 mmol) in DMF (5 mL) was added tributyl(vinyl)stannane (0.180 mL, 0.616 mmol). The mixture was stirred under atmosphere of nitrogen for 5 minutes then stirred overnight at 80° C. The reaction mixture was diluted with EtOAc and washed with water. The organic phase was dried over Na₂SO₄, filtered and concentrated in vacuo. The resulting residue was purified by silica gel chromatography (12 g ISCO column) using 0-20% EtOAc/heptanes gradient to afford 170 mg of product. 1-(4-fluoro-3-methyl-phenyl)-5-(methoxymethoxy)-2-tetrahydropyran-4-yl-3-vinyl-indole (93%). ¹H NMR (400 MHz, Chloroform-d) δ 7.36 (dd, J=2.4, 0.5 Hz, 1H), 6.98-6.82 (m, 4H), 6.65 (dd, J=8.8, 2.3 Hz, 1H), 6.55 (dd, J=8.8, 0.6 Hz, 1H), 5.46 (dd, J=17.7, 1.7 Hz, 1H), 5.11 (dd, J=11.5, 1.6 Hz, 1H), 4.98 (s, 2H), 3.84-3.70 (m, 2H), 3.29 (s, 3H), 3.08 (d, J=2.1 Hz, 1H), 2.13 (d, J=2.0 Hz, 3H), 1.40 (d, J=13.6 Hz, 2H).

### Step 2. Synthesis of trans-ethyl-2-(1-(4-fluoro-3-methylphenyl)-5-(methoxymethoxy)-2-(tetrahydro-2H-pyran-4-yl)-1H-indol-3-yl)cyclopropane-1-carboxylate (124)

To a suspension of 1-(4-fluoro-3-methyl-phenyl)-5-(methoxymethoxy)-2-tetrahydropyran-4-yl-3-vinyl-indole C39 (0.170 g, 0.430 mmol), (R,R)-PyBox (0.013 g, 0.043 mmol) and acridine-3,6-diamine; 10-methylacridin-10-ium-3,6-diamine; chloride (0.010 g, 0.021 mmol) in THE (10 mL) was added a solution of ethyl 2-diazoacetate (0.35 mL, 3.33 mmol) in toluene (3 mL). The reaction mixture was heated at 50° C. overnight. The mixture was concentrated, diluted with EtOAc and washed with water, dried over Na₂SO₄, filtered and concentrated in vacuo. The resulting residue was purified by silica gel chromatography (12 g ISCO column) using 0-40% EtOAc/heptanes gradient to afford 120 mg of trans cyclopropyl as the major isomer product. Trans-Ethyl-2-[1-(4-fluoro-3-methyl-phenyl)-5-(methoxymethoxy)-2-tetrahydropyran-4-yl-indol-3-yl]cyclopropane-carboxylate (58%). ESI-MS m/z calc. 481.2, found 482.0 (M+1)⁺.

### Step 3. Synthesis of trans-ethyl-2-(1-(4-fluoro-3-methylphenyl)-5-(methoxymethoxy)-2-(tetrahydro-2H-pyran-4-yl)-1H-indol-3-yl)cyclopropane-1-carboxylate (124)

To a solution of Trans-ethyl-2-[1-(4-fluoro-3-methyl-phenyl)-5-(methoxymethoxy)-2-tetrahydro-pyran-4-yl-indol-3-yl]cyclopropanecarboxylate C₄₀ (0.120 g, 0.249 mmol) in MeOH (1 mL) was added NaOH (1.00 mL of 1 M solution, 1.00 mmol). The mixture was heated at 50° C. for 1 hour. The mixture was concentrated in vacuo and acidified with 4 M HCl in dioxane and stirred for 1 h. Removed HCl and dioxane. The crude residue was purified by reverse phase HPLC to afford 2.8 mg of trans-2-[1-(4-fluoro-3-methyl-phenyl)-5-hydroxy-2-tetrahydropyran-4-yl-indol-3-yl]cyclopropanecarboxylic acid. 1H NMR (400 MHz, Chloroform-d) δ 7.01-6.78 (m, 4H), 6.44 (t, J=1.4 Hz, 2H), 3.87-3.70 (m, 2H), 3.11 (t, J=11.9 Hz, 2H), 2.76 (dt, J=12.3, 6.3 Hz, 1H), 2.48-2.36 (m, 1H), 2.24-2.06 (m, 4H), 2.05-1.90 (m, 1H), 1.89-1.75 (m, 2H), 1.58 (dt, J=9.0, 4.6 Hz, 1H), 1.42 (d, J=11.7 Hz, 2H), 1.36-1.21 (m, 1H). ESI-MS m/z calc. 409.2, found 408.6 (M+1)⁺.

### Compounds 125-139

Compounds in Table 9 (125-139) were prepared by an analogous method to that described for the preparation of compound 124. The appropriate vinyl indole intermediate was used in each example.

TABLE 9
Method of preparation, structure and physicochemical data for compounds 125-135

| | | | ¹H NMR; LCMS m/z |
|---|---|---|---|
| Compound | Product | Method | [M + H]⁺ |
| 125 | 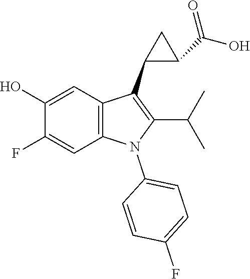 [SMILES: CC(C)c1c([C@H]2C[C@@H]2C(=O)O)c2cc(O)c(F)cc2n1-c1ccc(F)cc1] | From S19^{1,2,4} | ¹H NMR (400 MHz, Chloroform-d) δ 7.33-7.16 (m, 5H), 6.54 (d, J = 10.9 Hz, 1H), 3.17-2.98 (m, 1H), 2.67-2.62 (m, 1H), 2.12- 2.01 (m, 2H), 1.84-1.68 (m, 1H), 1.59-1.41 (m, 1H), 1.29 (t, J = 7.3 Hz, 7H). LCMS m/z 370.7 [M + H]⁺. |
| 126 | 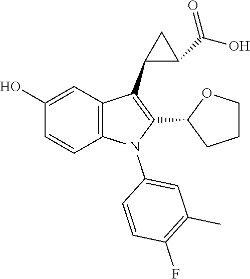 [SMILES: Cc1cc(-n2c([C@H]3CCCO3)c([C@H]3C[C@@H]3C(=O)O)c3cc(O)ccc32)ccc1F] | From S38^{1,3,4} | ¹H NMR (400 MHz, DMSO- d6) δ 7.39-7.11 (m, 3H), 6.93 (s, 1H), 6.68 (d, J = 8.7 Hz, 1H), 6.59 (dd, J = 8.8, 2.2 Hz, 1H), 4.91 (d, J = 8.8 Hz, 1H), 3.70-3.53 (m, 2H), 2.28 (s, 3H), 2.04 (d, J = 10.1 Hz, 1H), 1.97-1.71 (m, 2H), 1.63 (s, 1H), 1.41 (s, 1H), 1.22 (d, J = 12.8 Hz, 2H). LCMS m/z 396.2 [M + H]⁺. |
| 127 | 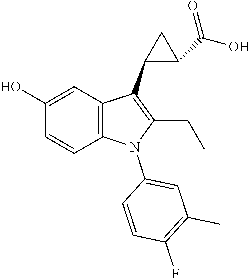 [SMILES: CCc1c([C@H]2C[C@@H]2C(=O)O)c2cc(O)ccc2n1-c1ccc(F)c(C)c1] | From S39^{,3,4} | LCMS m/z 354.6 [M + H]⁺. |
| 128 | 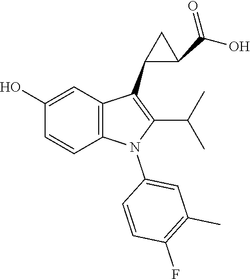 [SMILES: Cc1cc(-n2c(C(C)C)c([C@H]3C[C@H]3C(=O)O)c3cc(O)ccc32)ccc1F] | From S40^{1,3,4} | ¹H NMR (400 MHz, Methanol-d4) δ 7.26-7.04 (m, 5H), 6.58-6.40 (m, 2H), 2.489-2.47(m, 1H), 2.32 (d, J = 2.3 Hz, 3H), 2.14-2.11 (m, 1H), 1.76-1.73 (m, 1H), 1.59-1.56 (m, 1H), 1.25 (s, 2H), 1.16 (dd, J = 7.2, 2.4 Hz, 4H). LCMS m/z 368.2 [M + H]⁺. |
| 129 | 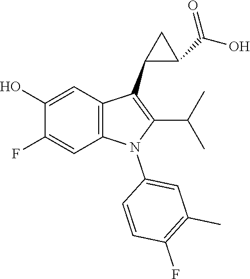 [SMILES: Cc1cc(-n2c(C(C)C)c([C@H]3C[C@@H]3C(=O)O)c3cc(O)c(F)cc32)ccc1F] | From S41^{1,3,4} | ¹H NMR (400 MHz, DMSO- d6) δ 12.36 (s, 1H), 9.20 (s, 1H), 7.41-7.26 (m, 2H), 7.22 (d, J = 4.2 Hz, 1H), 7.08 (d, J = 8.6 Hz, 1H), 6.47 (d, J = 11.3 Hz, 1H), 3.05-2.86 (m, 1H), 2.36-2.09 (m, 4H), 1.91-1.79 (m, 1H), 1.50 (s, 0H), 1.35 (ddd, J = 8.4, 6.7, 4.0 Hz, 1H), 1.26-1.14 (m, 8H). LCMS m/z 384.7 [M + H]⁺. |
| 130 | 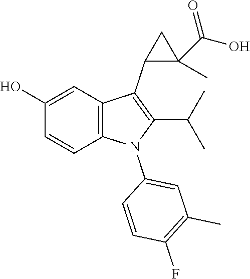 [SMILES: Cc1cc(-n2c(C(C)C)c(C3CC3(C)C(=O)O)c3cc(O)ccc32)ccc1F] | From S40^{1,3,4,6} | ¹H NMR (400 MHz, Chloroform-d) δ 7.15-7.08 (m, 3H), 7.04 (d, J = 1.8 Hz, 1H), 6.69-6.57 (m, 2H), 3.21-3.10 (m, 1H), 2.83- 2.76 (m, 1H), 2.33 (s, 3H), 2.02 (dd, J = 9.5, 4.0 Hz, 1H), 1.51-1.42 (m, 1H), 1.33-1.20 (m, 6H), 1.14 (s, 3H). LCMS m/z 381.6 [M + H]⁺. |
| 131 | 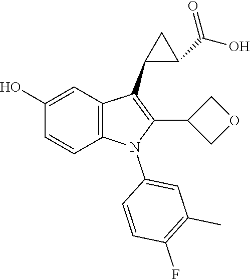 [SMILES: Cc1cc(-n2c(C3COC3)c([C@H]3C[C@@H]3C(=O)O)c3cc(O)ccc32)ccc1F] | From S42^{1,3,4} | ¹H NMR (400 MHz, Chloroform-d) δ 7.15-7.08 (m, 3H), 7.04 (d, J = 1.8 Hz, 1H), 6.69-6.57 (m, 2H), 3.21-3.10 (m, 1H), 2.83- 2.76 (m, 1H), 2.33 (s, 3H), 2.02 (dd, J = 9.5, 4.0 Hz, 1H), 1.51-1.42 (m, 1H), 1.33-1.20 (m, 6H), 1.14 (s, 3H). LCMS m/z 381.6 [M + H]⁺. |
| 132 | 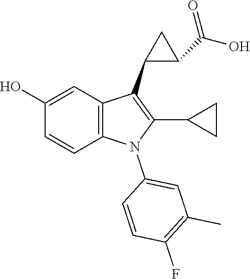 [SMILES: Cc1cc(-n2c(C3CC3)c([C@H]3C[C@@H]3C(=O)O)c3cc(O)ccc32)ccc1F] | From S43^{1,3,4} | ¹H NMR (300 MHz, Chloroform-d) δ 7.21-7.08 (m, 3H), 7.03 (d, J = 2.3 Hz, 1H), 6.92 (d, J = 8.7 Hz, 1H), 6.68 (dd, J = 8.7, 2.4 Hz, 1H), 2.60-2.54 (m, 1H), 2.33 (d, J = 1.6 Hz, 3H), 2.09- 1.96 (m, 1H), 1.79-1.66 (m, 2H), 1.63-1.46 (m, 1H), 0.91-0.73 (m, 2H), 0.68- 0.52 (m, 2H). LCMS m/z 365.1 [M + H]⁺. |
| 133 | 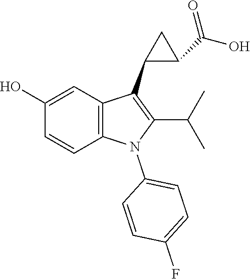 [SMILES: CC(C)c1c([C@H]2C[C@@H]2C(=O)O)c2cc(O)ccc2n1-c1ccc(F)cc1] | From S44^{1,3,4} | ¹H NMR (400 MHz, DMSO- d6) δ 12.34 (s, 1H), 8.83 (d, J = 1.8 Hz, 1H), 7.62-7.60 (m, 1H), 7.52-7.51 (m, 2H), 7.41 (t, J = 7.6 Hz, 1H), 6.92 (t, J = 1.7 Hz, 1H), 6.53 (dd, J = 8.7, 2.2 Hz, 1H), 6.48 (d, J = 8.6 Hz, 1H), 3.34 (s, 3H), 2.967-2.95 (m, 1H), 2.35- 2.33 (m, 1H), 1.85-1.82 (m, 1H), 1.56-1.47 (m, 1H), 1.39-1.37 (m, 1H), 1.24 (dd, J = 7.2, 3.0 Hz, 3H), 1.18 (dq, J = 7.1, 5.5, 5.0 Hz, 3H). LCMS m/z 353.0 [M + H]⁺. |
| 134 | 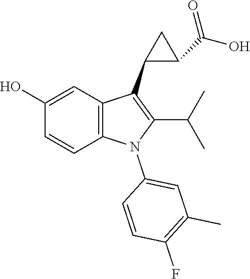 [SMILES: Cc1cc(-n2c(C(C)C)c([C@H]3C[C@@H]3C(=O)O)c3cc(O)ccc32)ccc1F] | From S40^{1,3,4} | ¹H NMR (400 MHz, DMSO- d6) δ 12.33 (s, 1H), 8.78 (s, 1H), 7.44-7.29 (m, 2H), 7.29-7.14 (m, 1H), 6.90 (dd, J = 2.1, 0.8 Hz, 1H), 6.65- 6.42 (m, 2H), 2.99 (p, J = 7.2 Hz, 1H), 2.42-2.22 (m, 4H), 1.82 (dd, J = 8.5, 4.4 Hz, 1H), 1.49 (dt, J = 8.9, 4.3 Hz, 1H), 1.36 (ddd, J = 8.2, 6.6, 3.8 Hz, 1H), 1.28-1.18 (m, 6H) ppm. LCMS m/z 368.4 [M + H]⁺. |
| 135 | 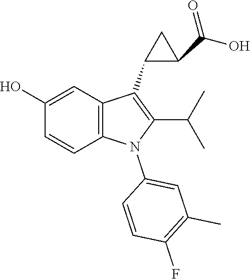 [SMILES: Cc1cc(-n2c(C(C)C)c([C@@H]3C[C@H]3C(=O)O)c3cc(O)ccc32)ccc1F] | From S40^{1,3,4} | ¹H NMR (400 MHz, DMSO- d6) δ 12.33 (s, 1H), 8.77 (s, 1H), 7.38-7.29 (m, 2H), 7.22-7.20(m, 1H), 6.90 (d, J = 2.0 Hz, 1H), 6.58-6.36 (m, 2H), 2.99 (p, J = 7.1 Hz, 1H), 2.40-2.27 (m, 4H), 1.90-1.83 (m, 1H), 1.60- 1.49 (m, 1H), 1.45-1.38 (m, 1H), 1.30-1.22 (m, 6H). LCMS m/z 367.5 [M + H]⁺. |

¹Cyclopropanation: ethyl 2-diazoacetate, (R,R)-PyBox, THF, toluene 50° C.
²Hydrolysis conditions: NaOH, MeOH
³Hydrolysis conditions: LiOH, MeOH, THF, H₂O
⁴Hydrogenation: H₂, Pd/C, MeOH
⁵Cyclopropanation: ethyl 2-diazopropanoate, (R,R)-PyBox, THF, toluene 50° C.
⁶Cyclopropanation with ethyl 2-diazopropanoate, Rh(OAc)₂ in dichloromethane.

Compound is mixture of stereoisomers.

### Compound 136

### Synthesis of cis-3-(1-(3,4-difluorophenyl)-5-hydroxy-2-(tetrahydro-2H-pyran-4-yl)-1H-indol-3-yl)cyclohexane-1-carboxylic acid (136)

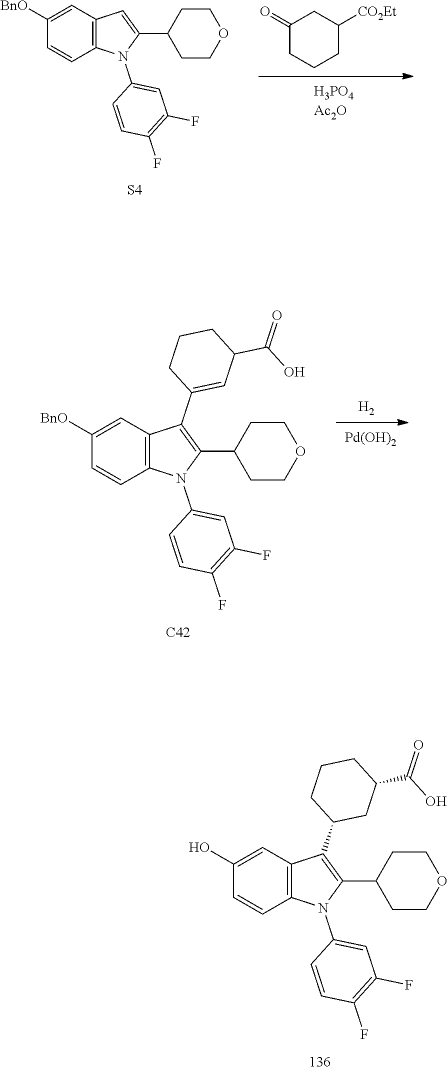

### Step 1. Synthesis of 3-(5-(benzyloxy)-1-(3,4-difluorophenyl)-2-(tetrahydro-2H-pyran-4-yl)-1H-indol-3-yl)cyclohex-2-ene-1-carboxylic acid (C42)

A solution of 5-benzyloxy-1-(3,4-difluorophenyl)-2-tetrahydropyran-4-yl-indole S4 (0.50 g, 1.14 mmol), ethyl 3-oxocyclohexanecarboxylate (0.39 g, 2.29 mmol), phosphoric acid (0.20 mL, 3.44 mmol) and acetic anhydride (0.20 mL, 2.12 mmol) in acetic acid (2.00 mL, 35.17 mmol) was heated at 110° C. for days in a sealable tube reactor. The solvent was removed under reduced pressure and sample was diluted with water (10 mL). The aqueous phase was extracted three times with EtOAc. The organic phase was dried (MgSO₄), filtered, and concentrated in vacuo. The resulting residue was purified by silica gel chromatography (40 g ISCO column) using 0-80% EtOAc/heptanes gradient to afford 213 mg of product. 3-[5-Benzyloxy-1-(3,4-difluorophenyl)-2-tetrahydropyran-4-yl-indol-3-yl]cyclohex-2-ene-1-carboxylic acid (33%). ESI-MS m/z calc. 543.2, found 544.5 (M+1)⁺.

### Step 2. Synthesis of cis-3-(1-(3,4-difluorophenyl)-5-hydroxy-2-(tetrahydro-2H-pyran-4-yl)-1H-indol-3-yl)cyclohexane-1-carboxylic acid (136)

To a solution of 3-[1-(3,4-difluorophenyl)-5-hydroxy-2-tetrahydropyran-4-yl-indol-3-yl]cyclohex-2-ene-1-carboxylic acid C42 (0.213 g, 0.469 mmol) in MeOH (8 mL) and EtOAc (2 mL) was added Pd(OH)₂ (0.08 g, 0.11 mmol). The system was evacuated and purged with hydrogen. The reaction mixture was stirred under an atmosphere of hydrogen for 18 hours. The crude mixture was filtered through a pad of celite and the filtrate was concentrated in vacuo. To afford 200 mg of product. 3-[1-(3,4-difluorophenyl)-5-hydroxy-2-tetrahydropyran-4-yl-indol-3-yl]cyclohexanecarboxylic acid (91%) as a racemic mixture of cis-isomers. ESI-MS m/z calc. 455.19, found 456.57 (M+1)⁺.

### Compounds 137 and 138

Compounds 137-138 were prepared from S4 using the same method as that described for the preparation of compound 136.

TABLE 10
Method of preparation, structure and physicochemical data for compounds 137-138

| Compound | Method/Product | Ketone | ¹H NMR; LCMS m/z [M + H]⁺ |
|---|---|---|---|
| 137 | 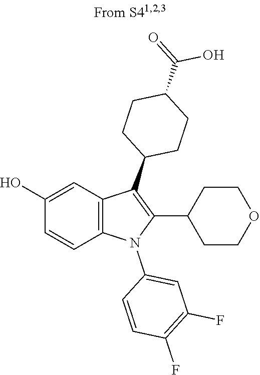 [SMILES: O=C(O)[C@H]1CC[C@H](c2c(C3CCOCC3)n(-c3ccc(F)c(F)c3)c3ccc(O)cc32)CC1] | 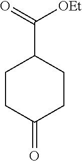 [SMILES: CCOC(=O)C1CCC(=O)CC1] | ¹H NMR (400 MHz, Methanol- d4) δ 7.49 (dt, J = 10.4, 8.8 Hz, 1H), 7.32-7.29 (m,, 1H), 7.18- 7.09 (m, 3H), 6.69 (d, J = 8.6 Hz, 1H), 6.60 (dd, J = 8.7, 2.3 Hz, 1H), 4.00 (dd, J = 11.6, 4.1 Hz, 2H), 3.06-3.04 (m, 1H), 2.84- 2.83 (m, 1H), 2.60-2.46 (m, 1H), 2.30-2.03 (m, 8H), 1.94 (d, J = 15.4 Hz, 2H), 1.77-1.45 (m, 4H). LCMS m/z 456.52 [M + H]⁺. |
| 138 | 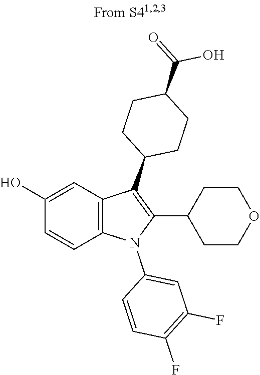 [SMILES: O=C(O)[C@H]1CC[C@@H](c2c(C3CCOCC3)n(-c3ccc(F)c(F)c3)c3ccc(O)cc32)CC1] | 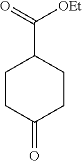 [SMILES: CCOC(=O)C1CCC(=O)CC1] | ¹H NMR (400 MHz, Methanol- d4) δ 7.49 (dt, J = 10.5, 8.8 Hz, 1H), 7.31 (ddd, J = 11.0, 7.2, 2.5 Hz, 1H), 7.14-7.10(m, 1H), 7.11- 7.04 (m, 1H), 6.67 (dd, J = 8.8, 0.5 Hz, 1H), 6.59 (dd, J = 8.7, 2.3 Hz, 1H), 3.99 (dd, J = 11.6, 4.2 Hz, 2H), 3.09-3.07 (m, 1H), 2.84-2.80 (m, 2H), 2.35 (t, J = 12.6 Hz, 4H), 2.14-2.00 (m, 2H), 1.81-1.64 (m, 6H). LCMS m/z 456.5 [M + H]⁺. |

¹Reductive alkylation: H₃PO₄, Ac₂O, AcOH, 110° C.
²Hydrogenation: H₂, Pd(OH)₂
³Hydrolysis conditions: LiOH, THF, MeOH, H₂O

### Compound 139

### Synthesis of trans-3-(1-(4-fluoro-3-methylphenyl)-5-hydroxy-2-(trifluoromethyl)-1H-indol-3-yl)-1-methylcyclobutane-1-carboxylic acid (139)

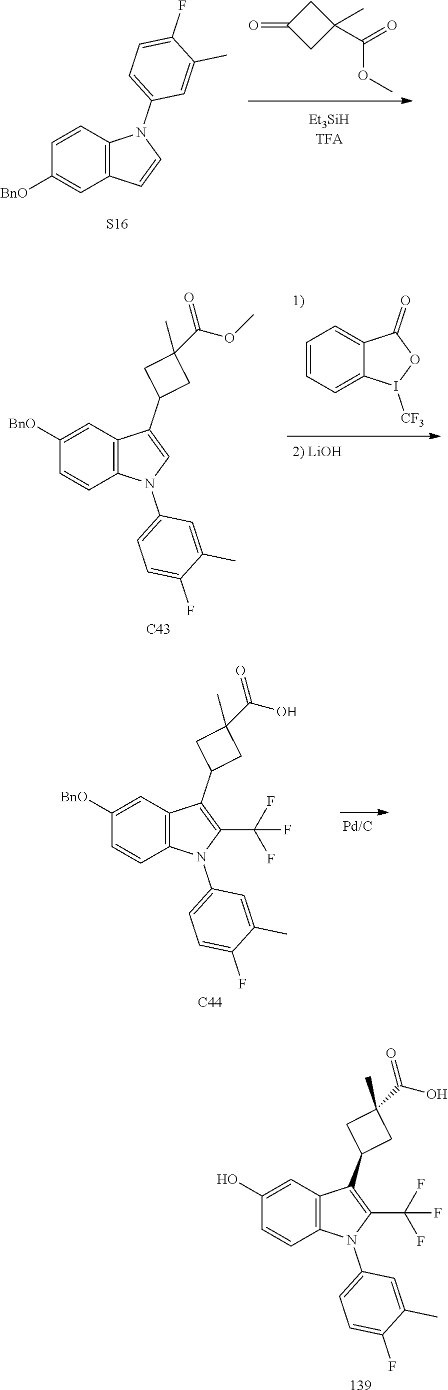

### Step 1. Synthesis of methyl 3-(5-(benzyloxy)-1-(4-fluoro-3-methylphenyl)-1H-indol-3-yl)-1-methylcyclobutane-1-carboxylate (C43)

To a solution of methyl 1-methyl-3-oxo-cyclobutanecarboxylate (0.48 g, 3.34 mmol), 5-benzyloxy-1-(4-fluoro-3-methyl-phenyl)indole S16 (0.75 g, 2.21 mmol) in CH₂Cl₂ (10 mL) was added trifluoroacetic acid (0.35 mL, 4.54 mmol) and triethylsilane (1.10 mL, 6.89 mmol). The reaction mixture was stirred at 50° C. for 48 hours. The mixture was diluted into water and dried over Na₂SO₄, filtered and concentrated in vacuo. The resulting residue was purified by silica gel chromatography using 0-50% EtOAc/heptanes gradient to afford 0.68 g of product. Methyl 3-[5-benzyloxy-1-(4-fluoro-3-methyl-phenyl)indol-3-yl]-1-methyl-cyclobutanecarboxylate (65%). ESI-MS m/z calc. 457.2, found 458.5 (M+1)⁺. The mixture of cis and trans isomers was taken onto next step without further purification.

### Step 2. Synthesis of 3-(5-(benzyloxy)-1-(4-fluoro-3-methylphenyl)-2-(trifluoromethyl)-1H-indol-3-yl)-1-methylcyclobutane-1-carboxylic acid (C44)

To a solution of methyl 3-[5-benzyloxy-1-(4-fluoro-3-methyl-phenyl)indol-3-yl]-1-methyl-cyclobutanecarboxylate C43 (0.68 g, 1.43 mmol) in CH₃CN (10 mL) was added 1-(trifluoromethyl)-1-3,2-benziodoxol-3-one (1.2 g, 2.278 mmol) (Togni's reagent). The reaction mixture was heated to 80° C. for 2 days. The solvent was removed under reduced pressure. The crude product was dissolved in EtOAc (10 mL) and washed with water. The organic phase was dried (MgSO₄), filtered, and concentrated in vacuo. The crude residue was purified by reverse phase flash chromatography (RF ISCO, C18 column, 30 g) eluting with CH₃CN/water (0-100%, 0.1% TFA) to afford 35 mg of product. Methyl 3-[5-benzyloxy-1-(4-fluoro-3-methyl-phenyl)-2-(trifluoromethyl)indol-3-yl]-1-methyl-cyclobutanecarboxylate (5%). The product was dissolved in MeOH (4.0 mL), THE (1.0 mL) and water (1.0 mL) and lithium hydroxide (0.05 g, 2.09 mmol) was added. The mixture was stirred at room temperature for 18 hours. The solvent was removed under reduced pressure. The crude residue was diluted in water (5 mL) and acidified with 6N HCl. The aqueous phase was extracted three times with EtOAc. The combined organic phases were dried over Na₂SO₄, filtered and concentrated in vacuo to afford 25 mg of product. 3-[5-benzyloxy-1-(4-fluoro-3-methyl-phenyl)-2-(trifluoromethyl)indol-3-yl]-1-methyl-cyclobutanecarboxylic acid. The crude product was used in following step without further purification.

### Step 3. Synthesis of trans-3-(1-(4-fluoro-3-methylphenyl)-5-hydroxy-2-(trifluoromethyl)-1H-indol-3-yl)-1-methylcyclobutane-1-carboxylic acid (139)

A solution of 3-[5-benzyloxy-1-(4-fluoro-3-methyl-phenyl)-2-(trifluoromethyl)indol-3-yl]-1-methyl-cyclobutanecarboxylic acid C44 (0.025 g, 0.049 mmol) in MeOH (3 mL) was purged with nitrogen. Pd/C (0.010 g, 0.009 mmol) was added followed by EtOAc (2 mL). The system was evacuated and purged with hydrogen. The reaction was stirred under an atmosphere of hydrogen for 18 hours. The crude mixture was filtered through a pad of celite and the filtrate was concentrated in vacuo. The crude residue was purified by reverse phase flash chromatography (RF ISCO, C18 column, 30 g) eluting with CH₃CN/water (0-100%, 0.1% TFA) to afford 12 mg of product. 3-[1-(4-Fluoro-3-methyl-phenyl)-5-hydroxy-2-(trifluoromethyl)indol-3-yl]-1-methyl-cyclobutanecarboxylic acid (53%). ¹H NMR (400 MHz, Methanol-d4) δ 7.33 (d, J=2.2 Hz, 1H), 7.24-7.04 (m, 4H), 6.84-6.76 (m, 2H), 4.13-3.98 (m, 1H), 2.92 (td, J=9.0, 2.7 Hz, 2H), 2.58 (td, J=10.0, 2.7 Hz, 2H), 2.33 (s, 3H), 1.52 (s, 3H). ESI-MS m/z calc. 421.13, found 422.23 (M+1)⁺.

### Compound 140

### 6-(1-(4-fluoro-3-methylphenyl)-5-hydroxy-2-(trifluoromethyl)-1H-indol-3-yl)spiro[3.3]heptane-2-carboxylic acid (140)

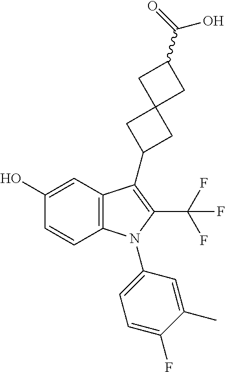 [SMILES: Cc1cc(-n2c(C(F)(F)F)c(C3CC4(CC(C(=O)O)C4)C3)c3cc(O)ccc32)ccc1F]

Compound 140 was prepared from 6-(5-(benzyloxy)-1-(4-fluoro-3-methylphenyl)-1H-indol-3-yl)spiro[3.3]heptane-2-carboxylic acid as described for C44 in the preparation of 139. Hydrogenation with Pd/C in EtOAc afforded final product. ¹H NMR (400 MHz, Methanol-d4) δ 7.26 (dd, J=2.2, 0.7 Hz, 1H), 7.22-7.09 (m, 3H), 6.86-6.72 (m, 2H), 3.94-3.78 (m, 1H), 3.07-3.07 (m, 1H), 2.69-2.41 (m, 6H), 2.35-2.28 (m, 5H). LCMS m/z 448.5 [M+H]⁺.

### Compound 141 and 142

### Synthesis of trans-3-(1-(3,4-difluorophenyl)-5-hydroxy-2-isopropyl-H-indol-3-yl)cyclobutane-1-carboxylic acid (141) and cis-3-(1-(3,4-difluorophenyl)-5-hydroxy-2-isopropyl-1H-indol-3-yl)cyclobutane-1-carboxylic acid (142)

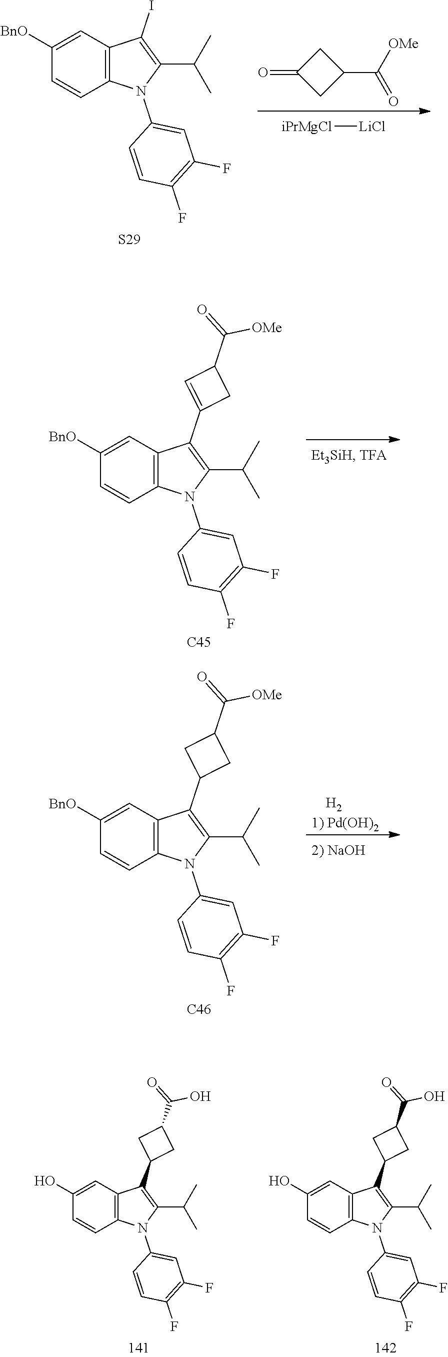

### Step 1. Synthesis of methyl 3-(5-(benzyloxy)-1-(3,4-difluorophenyl)-2-isopropyl-1H-indol-3-yl)cyclobut-2-ene-1-carboxylate (C46)

To a cold (0° C.) solution of 5-benzyloxy-1-(3,4-difluorophenyl)-3-iodo-2-isopropyl-indole S29 (0.37 g, 0.73 mmol) in THE (2 mL) was added iPrMgCl-LiCl (0.58 mL of 1.3 M solution, 0.75 mmol). The reaction mixture was stirred for 1 hour then slowly warmed to room temperature over 30 minutes. A solution of methyl 3-oxocyclobutanecarboxylate (0.10 g, 0.78 mmol) in THE (0.5 mL) was added to the reaction which was then stirred at room temperature for 2 hours. The reaction was quenched with H₂O and extracted with CH₂Cl₂. The organic phase was concentrated in vacuo and used without further purification in the next step.

To a solution of the crude product dissolved in CH₂Cl₂ (5 mL) was added triethylamine (0.16 mL, 2.08 mmol) and triethylsilane (0.45 mL, 2.82 mmol). The reaction mixture was stirred at room temperature overnight and then concentrated in vacuo. The resulting residue was purified by silica gel chromatography using 0-10% MeOH/CH₂Cl₂ gradient to afford 120 mg of product. Methyl 3-[5-benzyloxy-1-(3,4-difluorophenyl)-2-isopropyl-indol-3-yl]cyclobutanecarboxylate (34%) ESI-MS m/z calc. 489.2, found 490.3 (M+1)⁺.

### Step 2. Synthesis of trans-3-(1-(3,4-difluorophenyl)-5-hydroxy-2-isopropyl-1H-indol-3-yl)cyclobutane-1-carboxylic acid (141) and cis-3-(1-(3,4-difluorophenyl)-5-hydroxy-2-isopropyl-1H-indol-3-yl)cyclobutane-1-carboxylic acid (142)

To a solution of methyl 3-[5-benzyloxy-1-(3,4-difluorophenyl)-2-isopropyl-indol-3-yl]cyclobutanecarboxylate C46 (0.12 g, 0.25 mmol) in EtOAc (10 mL) was added Pd(OH)₂ (0.03 g, 0.21 mmol) The reaction mixture was evacuated and purged with hydrogen and stirred under a hydrogen atmosphere for 2 hours. The crude mixture was filtered through a pad of celite and the filtrate was concentrated in vacuo to afford 80 mg of crude product that was used without further purification in the next step. methyl 3-[1-(3,4-difluorophenyl)-5-hydroxy-2-isopropyl-indol-3-yl]cyclobutanecarboxylate (82%). ESI-MS m/z calc. 399.2, found 400.5 (M+1)⁺.

To a solution of methyl 3-[1-(3,4-difluorophenyl)-5-hydroxy-2-isopropyl-indol-3-yl]cyclobutanecarboxylate (80 mg) in MeOH (10 mL) was added NaOH (0.50 mL of 3 M solution, 1.50 mmol). The reaction mixture was stirred at room temperature for 1 hour and concentrated in vacuo. The crude residue was purified by reverse phase flash chromatography eluting with CH₃CN/water (0-100%, 0.1% TFA) to afford 18.6 mg of 3-[1-(3,4-difluorophenyl)-5-hydroxy-2-isopropyl-indol-3-yl]cyclobutanecarboxylic acid (37%). ¹H NMR (400 MHz, Chloroform-d) δ 7.39-7.30 (m, 2H), 7.22-7.12 (m, 1H), 7.07 (ddd, J=8.8, 4.0, 1.8 Hz, 1H), 6.80-6.75 (m, 1H), 6.68 (dd, J=8.7, 2.4 Hz, 1H), 4.29 (t, J=9.4 Hz, 1H), 3.45 (t, J=9.8 Hz, 1H), 3.18-3.02 (m, 2H), 2.99-2.87 (m, 1H), 2.81-2.70 (m, 2H), 1.28 (d, J=7.2 Hz, 6H). ESI-MS m/z calc. 385.1, found 386.0 (M+1)⁺ and 18 mg of 3-[1-(3,4-difluorophenyl)-5-hydroxy-2-isopropyl-indol-3-yl]cyclobutanecarboxylic acid (18.5 mg, 37%), ¹H NMR (400 MHz, Chloroform-d) δ 7.74 (dd, J=2.0, 0.9 Hz, 1H), 7.38-7.32 (m, 1H), 7.15 (ddd, J=10.5, 7.1, 2.5 Hz, 1H), 7.07 (ddd, J=8.8, 3.9, 1.9 Hz, 1H), 6.82-6.75 (m, 2H), 3.94 (d, J=9.4 Hz, 1H), 3.16 (d, J=10.7 Hz, 2H), 3.02-2.88 (m, 1H), 2.68-2.56 (m, 2H), 1.30 (d, J=7.2 Hz, 6H). ESI-MS m/z calc. 385.1, found 386.0 (M+1)⁺.

### Compounds 143-146

Compounds 143-146 (Table 11) were prepared from the appropriate ketone and indole iodide intermediate using the method described for the preparation of compound 141 and 142.

TABLE 11
Method of preparation, structure and physicochemical data for compounds 143-146

| Compound | Method/Product | Ketone | ¹H NMR; LCMS m/z [M + H]⁺ |
|---|---|---|---|
| 143 | 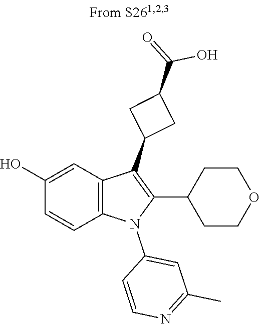 [SMILES: Cc1cc(-n2c(C3CCOCC3)c([C@H]3C[C@@H](C(=O)O)C3)c3cc(O)ccc32)ccn1] | 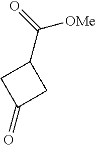 | ¹H NMR (400 MHz, Methanol- d4) δ 8.59 (d, J = 5.4 Hz, 1H), 8.44 (s, 2H), 7.55 (s, 1H), 7.29 (s, 1H), 7.19 (d, J = 5.6 Hz, 1H), 6.85 (d, J = 8.8 Hz, 1H), 6.63 (d, J = 8.7 Hz, 1H), 3.99 (d, J = 10.6 Hz, 3H), 2.99 (d, J = 10.8 Hz, 1H), 2.63 (s, 3H), 2.54 (d, J = 9.4 Hz, 2H), 2.12 (d, J = 13.0 Hz, 2H), 1.72 (d, J = 13.0 Hz, 2H). LCMS m/z 407.7 [M + H]⁺. |
| 144 | 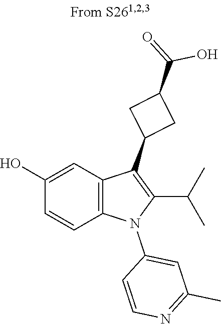 [SMILES: Cc1cc(-n2c(C(C)C)c([C@H]3C[C@@H](C(=O)O)C3)c3cc(O)ccc32)ccn1] | 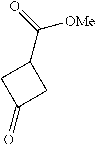 | ¹H NMR (400 MHz, Methanol- d4) δ 8.57 (d, J = 5.4 Hz, 1H), 7.52 (d, J = 2.3 Hz, 1H), 7.27 (d, J = 2.0 Hz, 1H), 7.22-7.16 (m, 1H), 6.84 (d, J = 8.8 Hz, 1H), 6.62 (dd, J = 8.8, 2.3 Hz, 1H), 4.03-3.84 (m, 1H), 3.19 (d, J = 9.3 Hz, 1H), 3.07-2.88 (m, 3H), 2.62 (s, 3H), 2.57-2.42 (m, 1H), 1.34 (d, J = 7.2 Hz, 6H). LCMS m/z 365.7 [M + H]⁺. |
| 145 | 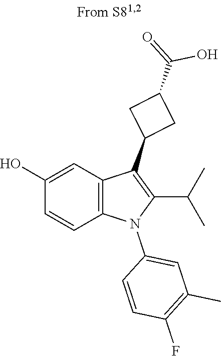 [SMILES: Cc1cc(-n2c(C(C)C)c([C@H]3C[C@H](C(=O)O)C3)c3cc(O)ccc32)ccc1F] | 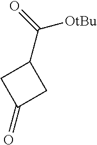 [SMILES: CC(C)(C)OC(=O)C1CC(=O)C1] | ¹H NMR (300 MHz, Methanol- d4) δ 7.30 (dd, J = 2.2, 0.7 Hz, 1H), 7.25-7.02 (m, 3H), 6.68- 6.50 (m, 2H), 4.36-4.11 (m, 1H), 3.12-2.82 (m, 3H), 2.73- 2.51 (m, 3H), 2.33 (d, J = 2.0 Hz, 3H), 1.25 (d, J = 7.2 Hz, 6H). LCMS m/z 382.3 [M + H]⁺. |
| 146 | 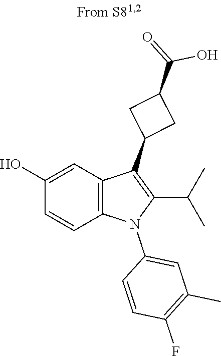 [SMILES: Cc1cc(-n2c(C(C)C)c([C@H]3C[C@@H](C(=O)O)C3)c3cc(O)ccc32)ccc1F] | 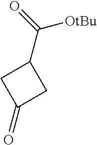 [SMILES: CC(C)(C)OC(=O)C1CC(=O)C1] | ¹H NMR (300 MHz, Methanol- d4) δ 7.47 (d, J = 2.1 Hz, 1H), 7.29-7.00 (m, 3H), 6.69-6.44 (m, 2H), 3.91 (tt, J = 10.3, 8.4 Hz, 1H), 3.18 (dq, J = 10.7, 8.3 Hz, 1H), 3.06-2.86 (m, 3H), 2.51 (qd, J = 8.4, 2.5 Hz, 2H), 2.32 (d, J = 2.0 Hz, 3H), 1.26 (d, J = 7.2 Hz, 6H). LCMS m/z 382.3 [M + H]⁺. |
| 147 | 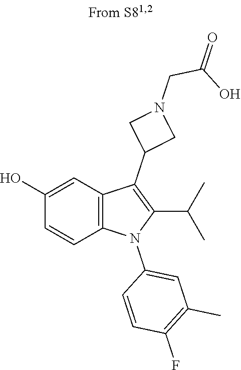 [SMILES: Cc1cc(-n2c(C(C)C)c(C3CN(CC(=O)O)C3)c3cc(O)ccc32)ccc1F] | 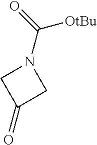 [SMILES: CC(C)(C)OC(=O)N1CC(=O)C1] | ¹H NMR (400 MHz, DMSO-d6) δ 9.22 (s, 1H), 7.59 (s, 1H), 7.38- 7.30 (m, 1H), 7.28-7.23 (m, 1H), 7.21-7.11 (m, 1H), 6.66- 6.52 (m, 2H), 4.68-4.55 (m, 1H), 4.53-4.36 (m, 2H), 4.21- 4.12 (m, 2H), 4.08-3.96 (m, 2H), 2.91-2.77 (m, 1H), 2.30 (s, 3H), 1.22 (d, J = 7.1 Hz, 6H). LCMS m/z 397.3 [M + H]⁺. |

¹iPrMgCl—LiCl, THF, 0° C.
²Et₃SiH, TFA, CH₂Cl₂
³Hydrolysis conditions: NaOH, MeOH
⁴Alkylation of the intermediate amine 3-(azetidin-3-yl)-5-benzyloxy-1-(4-fluoro-3-methyl-phenyl)-2-isopropyl-indole with benzyl 2-chloroacetate, then benzyl group removal by hydrogenolysis afforded the product.

### Compounds 148 and 149

### Synthesis of 2-fluoro-6-(1-(4-fluoro-3-methylphenyl)-5-hydroxy-2-isopropyl-1H-indol-3-yl)spiro[3.3]heptane-2-carboxylic acid (148) and 2-fluoro-6-(1-(4-fluoro-3-methylphenyl)-5-hydroxy-2-isopropyl-1H-indol-3-yl)spiro[3.3]heptane-2-carboxylic acid (149)

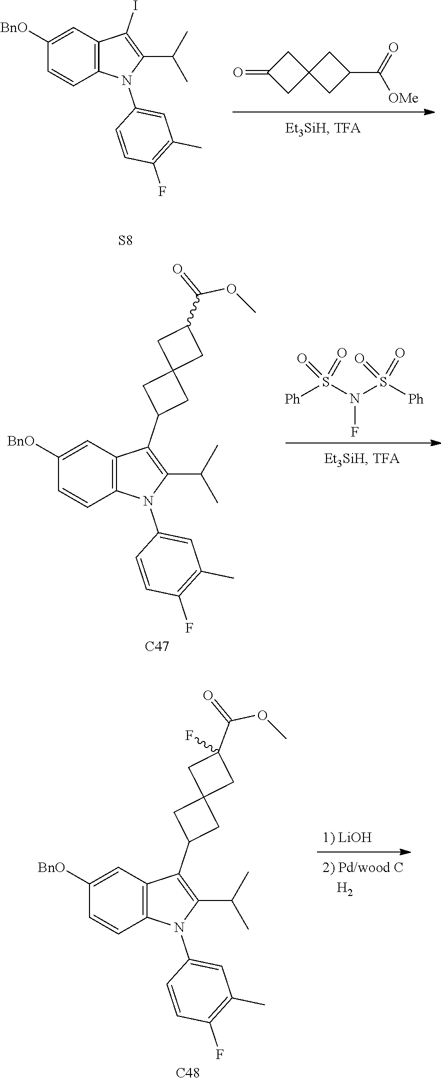

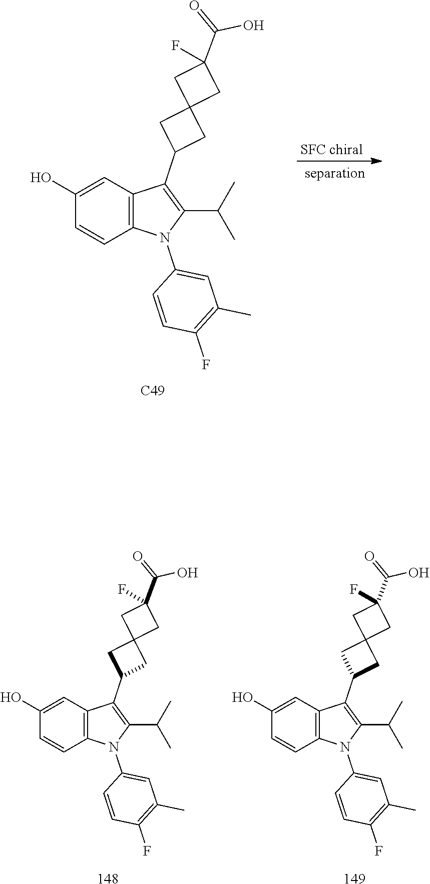

### Step 1. Synthesis of methyl 6-(5-(benzyloxy)-1-(4-fluoro-3-methylphenyl)-2-isopropyl-1H-indol-3-yl)spiro[3.3]heptane-2-carboxylate (C47)

A solution of 5-benzyloxy-1-(4-fluoro-3-methyl-phenyl)-2-isopropyl-indole S8 (0.80 g, 2.14 mmol), methyl 2-oxospiro[3.3]heptane-6-carboxylate (0.56 g, 3.33 mmol) in CH₂Cl₂ (12.0 mL) was added trifluoroacetic acid (0.34 mL, 4.41 mmol) and triethylsilane (1.05 mL, 6.57 mmol). The reaction mixture was stirred at room temperature for 3 days. The reaction mixture was diluted with water, dried over Na₂SO₄, filtered and concentrated in vacuo. The resulting residue was purified by silica gel chromatography (40 g ISCO column) using 0-50% EtOAc/heptanes gradient to afford 0.94 g of product. Methyl 6-[5-benzyloxy-1-(4-fluoro-3-methyl-phenyl)-2-isopropyl-indol-3-yl]spiro[3.3]heptane-2-carboxylate (82%). ¹H NMR (400 MHz, Chloroform-d) δ 7.49-7.44 (m, 2H), 7.43-7.35 (m, 2H), 7.36-7.29 (m, 2H), 7.11-7.01 (m, 3H), 6.81-6.62 (m, 2H), 5.13 (s, 2H), 3.86-3.74 (m, 1H), 3.71 (s, 3H), 3.11 (p, J=8.5 Hz, 1H), 2.94 (h, J=7.2 Hz, 1H), 2.70 (dt, J=25.0, 10.7 Hz, 2H), 2.48 (dd, J=8.5, 1.3 Hz, 2H), 2.40 (dd, J=11.5, 8.5 Hz, 1H), 2.35 (d, J=5.3 Hz, 5H), 1.23 (dt, J=7.3, 1.6 Hz, 6H). ESI-MS m/z calc. 525.27, found 525.21 (M+1)⁺.

### Step 2. Synthesis of methyl 6-(5-(benzyloxy)-1-(4-fluoro-3-methylphenyl)-2-isopropyl-1H-indol-3-yl)-2-fluorospiro[3.3]heptane-2-carboxylate (C48)

To a cold (−78° C.) solution of methyl 6-[5-benzyloxy-1-(4-fluoro-3-methyl-phenyl)-2-isopropyl-indol-3-yl]spiro[3.3]heptane-2-carboxylate C47 (0.85 g, 1.59 mmol) in tetrahydrofuran (20 mL) was added (diisopropylamino)lithium (1.05 mL of 2 M, 2.10 mmol). The mixture was warmed to −10° C. and stirred for 30 minutes. The mixture was cooled to −78° C. and N-(benzenesulfonyl)-N-fluoro-benzenesulfonamide (0.65 g, 2.06 mmol) in THE (2.0 mL) was added and the mixture was slowly warmed to room temperature. The reaction was quenched with aqueous saturated NH₄Cl solution and extracted with EtOAc. The resulting residue was purified by silica gel chromatography (40 g ISCO column) using 0-50% EtOAc/heptanes gradient to afford 0.5 g of product. Methyl 6-[5-benzyloxy-1-(4-fluoro-3-methyl-phenyl)-2-isopropyl-indol-3-yl]-2-fluoro-spiro[3.3]heptane-2-carboxylate (58%). ¹H NMR (400 MHz, Chloroform-d) δ 7.47 (dd, J=8.1, 1.5 Hz, 2H), 7.44-7.35 (m, 2H), 7.35-7.30 (m, 2H), 7.19-6.99 (m, 3H), 6.92-6.65 (m, 2H), 5.13 (s, 2H), 3.84-379 (m, 4H), 2.96 (ddd, J=14.7, 9.4, 5.5 Hz, 2H), 2.85-2.58 (m, 5H), 2.59-2.41 (m, 2H), 2.33 (d, J=2.0 Hz, 3H), 1.23 (dd, J=7.2, 1.4 Hz, 6H).

### Step 3. Synthesis of 2-fluoro-6-(1-(4-fluoro-3-methylphenyl)-5-hydroxy-2-isopropyl-1H-indol-3-yl)spiro[3.3]heptane-2-carboxylic acid (C49)

To a solution of methyl 6-[5-benzyloxy-1-(4-fluoro-3-methyl-phenyl)-2-isopropyl-indol-3-yl]-2-fluoro-spiro[3.3]heptane-2-carboxylate C48 (0.50 g, 0.92 mmol) in MeOH (10.0 mL), THE (3.0 mL) and H₂O (1.5 mL) was added lithium hydroxide (0.65 g, 15.49 mmol). The reaction mixture was stirred at room temperature for 18 hours. The solvent was evaporated under reduced pressure and the white solid was dissolved in water (10 mL) and slowly acidified with HCl (12.0 mL of 2 M, 24.0 mmol). The aqueous phase was extracted three times with EtOAc dried (MgSO₄), filtered, and concentrated in vacuo to afford 480 mg of product. 6-[5-benzyloxy-1-(4-fluoro-3-methyl-phenyl)-2-isopropyl-indol-3-yl]-2-fluoro-spiro[3.3]heptane-2-carboxylic acid (95%). ESI-MS m/z calc. 529.24, found 530.51 (M+1)⁺. A solution of the product (480.0 mg, 0.9063 mmol) in EtOAc (20.0 mL) was purged with nitrogen. To the mixture was added Pd/wood carbon (0.24 g of 10% w/w, 0.09 mmol) and the mixture was evacuated and purged with hydrogen. The crude mixture was filtered through a pad of celite and the filtrate was concentrated in vacuo. The resulting residue was purified by silica gel chromatography (4 g ISCO column) using 0-10% MeOH/heptanes gradient to afford 0.38 g of product (89%). The crude product was submitted for SFC purification to afford 67.5 mg of 2-fluoro-6-(1-(4-fluoro-3-methylphenyl)-5-hydroxy-2-isopropyl-1H-indol-3-yl)spiro[3.3]heptane-2-carboxylic acid (148), ¹H NMR (400 MHz, Chloroform-d) δ 7.20 (d, J=2.3 Hz, 1H), 7.15-6.99 (m, 3H), 6.70 (d, J=8.7 Hz, 1H), 6.61 (dd, J=8.7, 2.4 Hz, 1H), 3.86-3.74 (m, 1H), 3.10-2.41 (m, 9H), 2.32 (d, J=2.1 Hz, 3H), 1.26-1.15 (m, 6H), ESI-MS m/z calc. 439.19, found 440.55 (M+1)⁺ and 61 mg of 2-fluoro-6-(1-(4-fluoro-3-methylphenyl)-5-hydroxy-2-isopropyl-1H-indol-3-yl)spiro[3.3]heptane-2-carboxylic acid (149). 1H NMR (400 MHz, Chloroform-d) δ 7.20 (d, J=2.3 Hz, 1H), 7.15-7.00 (m, 3H), 6.70 (d, J=8.7 Hz, 1H), 6.60 (dd, J=8.7, 2.4 Hz, 1H), 3.81 (tt, J=10.1, 8.4 Hz, 1H), 3.09-2.41 (m, 9H), 2.32 (d, J=1.9 Hz, 3H), 1.23 (dt, J=7.2, 1.2 Hz, 6H).

### Compound 150

### 6-(1-(4-fluoro-3-methylphenyl)-5-hydroxy-2-(trifluoromethyl)-1H-indol-3-yl)spiro[3.3]heptane-2-carboxylic acid (150)

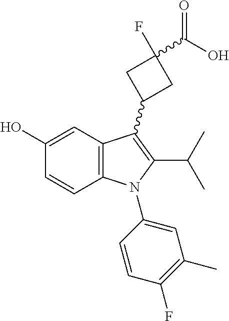 [SMILES: Cc1cc(-n2c(C(C)C)c(C3CC(F)(C(=O)O)C3)c3cc(O)ccc32)ccc1F]

Compound 150 was prepared from methyl 3-(5-(benzyloxy)-1-(4-fluoro-3-methylphenyl)-2-isopropyl-1H-indol-3-yl)cyclobutane-1-carboxylate as described for C₄₈ in the preparation of 148. Ester hydrolysis with sodium hydroxide in methanol was followed by hydrogenation with Pd/C in EtOAc to afford final product. Compound 150 is isolated as single stereoisomer with unknown absolute configuration. ¹H NMR (400 MHz, Chloroform-d) δ 7.71-7.60 (m, 1H), 7.17-7.03 (m, 3H), 6.80-6.68 (m, 2H), 4.41 (p, J=9.6 Hz, 1H), 3.54 (dt, J=28.6, 11.6 Hz, 2H), 2.95 (p, J=7.2 Hz, 1H), 2.76 (dddd, J=20.2, 11.5, 8.8, 3.3 Hz, 2H), 2.33 (d, J=2.0 Hz, 3H), 1.29 (dd, J=7.2, 1.7 Hz, 6H). LCMS m/z 400.3 [M+H]⁺.

### Compound 151

### Synthesis of 3-(1-(4-fluoro-3-methylphenyl)-5-hydroxy-2-isopropyl-1H-indol-3-yl)-2-methoxy-2-methylpropanoic acid (151)

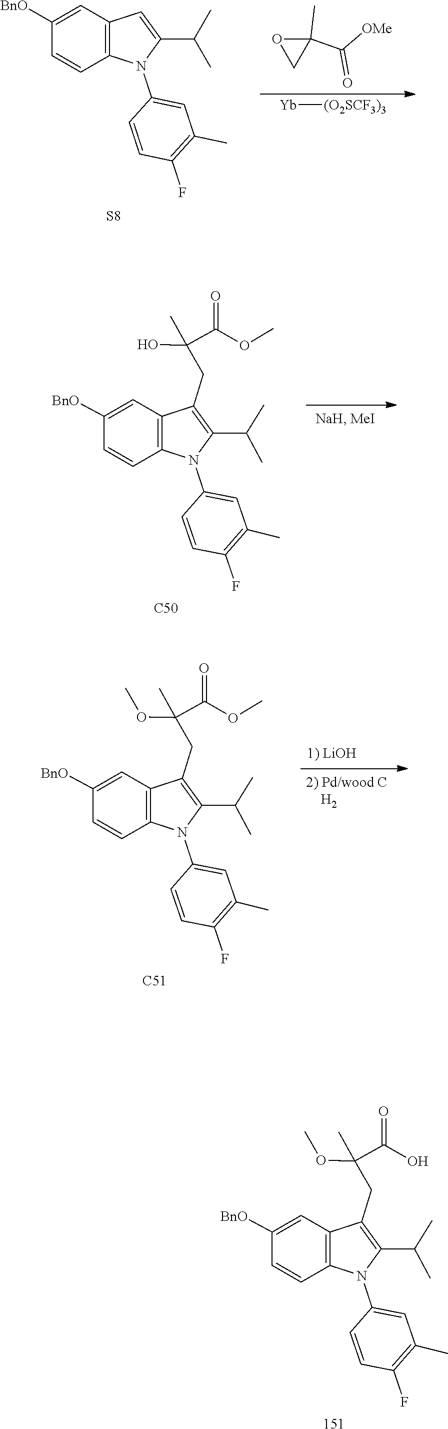

### Step 1. Synthesis of methyl 3-(5-(benzyloxy)-1-(4-fluoro-3-methylphenyl)-2-isopropyl-1H-indol-3-yl)-2-hydroxy-2-methylpropanoate (C50)

To a solution of 5-benzyloxy-1-(4-fluoro-3-methyl-phenyl)-2-isopropyl-indole S8 (0.50 g, 1.34 mmol) in 1,2-dichloroethane (7.0 mL) was added methyl 2-methyloxirane-2-carboxylate (0.43 mL, 4.02 mmol) and tris(trifluoromethylsulfonyloxy)ytterbium (0.40 g, 0.65 mmol). The reaction mixture was heated at 80° C. for 16 hours. The reaction was quenched with aqueous saturated NaHCO₃ solution and extracted with CH₂Cl₂, The combined organic phases were dried (MgSO₄), filtered, and concentrated in vacuo. The resulting residue was purified by silica gel chromatography (40 g ISCO column) using 0-40% EtOAc/heptanes to afford 275 mg of product. Methyl 3-[5-benzyloxy-1-(4-fluoro-3-methyl-phenyl)-2-isopropyl-indol-3-yl]-2-hydroxy-2-methyl-propanoate (42%). ¹H NMR (400 MHz, Chloroform-d) δ 7.54-7.48 (m, 2H), 7.47-7.38 (m, 2H), 7.38-7.30 (m, 1H), 7.23-7.06 (m, 4H), 6.87-6.76 (m, 1H), 6.64 (d, J=8.8 Hz, 1H), 5.13 (s, 2H), 3.74 (d, J=2.8 Hz, 3H), 3.43-3.10 (m, 3H), 2.35 (dd, J=4.0, 2.0 Hz, 3H), 1.60 (s, 3H), 1.15 (ddd, J=11.3, 7.2, 1.2 Hz, 6H).

### Step 2. Synthesis of methyl 3-(5-(benzyloxy)-1-(4-fluoro-3-methylphenyl)-2-isopropyl-1H-indol-3-yl)-2-methoxy-2-methylpropanoate (C51)

To a cold (0° C.) solution of methyl 3-[5-benzyloxy-1-(4-fluoro-3-methyl-phenyl)-2-isopropyl-indol-3-yl]-2-hydroxy-2-methyl-propanoate C50 (0.11 g, 0.22 mmol) in DMF (2 mL) was added sodium hydride (0.020 g of 60% w/w, 0.500 mmol). The reaction mixture was stirred for 30 minutes. To the mixture was added iodomethane (0.030 mL, 0.482 mmol) and the mixture was stirred at room temperature for 12 hours. The reaction was quenched with aqueous saturated NH₄Cl solution and extracted with EtOAc. The organic phase was washed with brine, dried (MgSO₄), filtered, and concentrated in vacuo. The resulting residue was purified by silica gel chromatography (40 g ISCO column) using 0-40% EtOAc/heptanes gradient to afford 95 mg of product. Methyl 3-[5-benzyloxy-1-(4-fluoro-3-methyl-phenyl)-2-isopropyl-indol-3-yl]-2-methoxy-2-methyl-propanoate (84%). ¹H NMR (400 MHz, Chloroform-d) δ 7.56-7.45 (m, 2H), 7.45-7.39 (m, 2H), 7.38-7.29 (m, 1H), 7.22 (d, J=2.4 Hz, 1H), 7.19-7.08 (m, 3H), 6.87-6.72 (m, 1H), 6.63 (d, J=8.7 Hz, 1H), 5.12 (s, 2H), 3.76 (d, J=0.5 Hz, 3H), 3.43-3.34 (m, 1H), 3.29 (s, 3H), 3.24-3.13 (m, 2H), 2.34 (d, J=1.9 Hz, 3H), 1.19-1.02 (m, 6H).

### Step 3. Synthesis of 3-(1-(4-fluoro-3-methylphenyl)-5-hydroxy-2-isopropyl-1H-indol-3-yl)-2-methoxy-2-methylpropanoic acid (151)

To a solution of methyl 3-[5-benzyloxy-1-(4-fluoro-3-methyl-phenyl)-2-isopropyl-indol-3-yl]-2-methoxy-2-methyl-propanoate C51 (0.090 mg, 0.178 mmol) in MeOH (2.0 mL), THE (0.6 mL) and H₂O (0.40 mL) was added lithium hydroxide (0.128 g, 3.050 mmol). The reaction mixture was stirred at room temperature for 18 hours. The solvent was evaporated under reduced pressure and the white solid was dissolved in water (10 mL) and slowly acidified with HCl (1.8 mL of 2 M, 3.6 mmol). The aqueous phase was extracted three times with EtOAc. The organic phase was dried (MgSO₄), filtered, and concentrated in vacuo to afford 80 mg of product. 3-[5-benzyloxy-1-(4-fluoro-3-methyl-phenyl)-2-isopropyl-indol-3-yl]-2-methoxy-2-methyl-propanoic acid (91%). ¹H NMR (400 MHz, Chloroform-d) δ 7.53-7.45 (m, 2H), 7.45-7.35 (m, 2H), 7.35-7.27 (m, 1H), 7.21-7.07 (m, 4H), 6.79 (dd, J=8.8, 2.5 Hz, 1H), 6.62 (d, J=8.8 Hz, 1H), 5.12 (s, 2H), 3.38-3.22 (m, 4H), 2.40-2.26 (m, 2H), 1.16-1.03 (m, 6H). To a nitrogen purged solution of 3-[5-benzyloxy-1-(4-fluoro-3-methyl-phenyl)-2-isopropyl-indol-3-yl]-2-methoxy-2-methyl-propanoic acid (0.070 g, 0.143 mmol) in EtOAc (2.0 mL) was added Pd on carbon (0.039 g of 10% w/w, 0.015 mmol) and the mixture was evacuated and filled with hydrogen. The mixture was stirred under an atmosphere of hydrogen for 2 h. The crude mixture was filtered through a pad of celite, filtered and concentrated in vacuo. and purified using ISCO (4 g gold The resulting residue was purified by silica gel chromatography (4 g ISCO column) using 0-10% MeOH/CH₂Cl₂. 3-[1-(4-fluoro-3-methyl-phenyl)-5-hydroxy-2-isopropyl-indol-3-yl]-2-methoxy-2-methyl-propanoic acid (33%). ¹H NMR (400 MHz, Chloroform-d) δ 7.17-7.07 (m, 3H), 7.05 (d, J=2.3 Hz, 1H), 6.67-6.51 (m, 2H), 3.35 (s, 3H), 3.29 (q, J=7.2 Hz, 1H), 3.22-3.17 (m, 2H), 2.33-2.27 (m, 3H), 1.54 (s, 3H), 1.15-1.07 (m, 6H). ESI-MS m/z calc. 399.18, found 400.31 (M+1)⁺.

### Compound 152

### 3-[1-(4-fluoro-3-methyl-phenyl)-5-hydroxy-2-isopropyl-indol-3-yl]-2-hydroxy-2-methyl-propanoic acid (152)

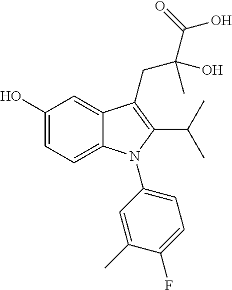 [SMILES: Cc1cc(-n2c(C(C)C)c(CC(C)(O)C(=O)O)c3cc(O)ccc32)ccc1F]

Compound 152 was prepared from 5-benzyloxy-1-(4-fluoro-3-methyl-phenyl)-2-isopropyl-indole S8 as described for C51 in the preparation of 151. Ester hydrolysis with lithium hydroxide in methanol, THE and water was followed by hydrogenation with Pd/C (wood) in EtOAc to afford final product. ¹H NMR (400 MHz, Chloroform-d) δ 7.23-7.08 (m, 3H), 7.04 (d, J=2.2 Hz, 1H), 6.75-6.52 (m, 2H), 3.48 (d, J=14.9 Hz, 1H), 3.31 (p, J=7.3 Hz, 1H), 3.17 (d, J=14.9 Hz, 1H), 2.34 (d, J=2.1 Hz, 3H), 1.64 (s, 3H), 1.16 (t, J=6.5 Hz, 6H). LCMS m/z 386.3 [M+H]⁺.

### Preparation 153

### Synthesis of 2-(1-(4-fluoro-3-methylphenyl)-5-hydroxy-2-isopropyl-1H-indol-3-yl)acetic acid (153)

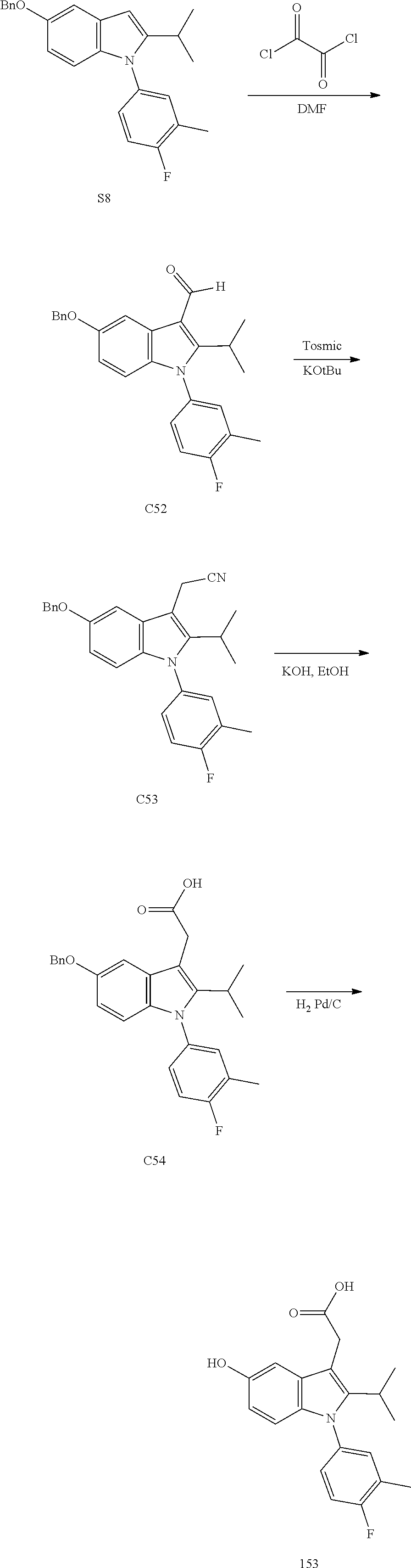

### Step 1. Synthesis of 5-(benzyloxy)-1-(4-fluoro-3-methylphenyl)-2-isopropyl-1H-indole-3-carbaldehyde (C52)

To as solution of oxalyl chloride (13.0 mL of 2 M solution, 26.0 mmol) in CH₂Cl₂ at 0° C. was added DMF (13.0 mL, 167.9 mmol). The suspension was stirred at 0° C. for 10 minutes. 5-benzyloxy-1-(4-fluoro-3-methyl-phenyl)-2-isopropyl-indole S8 (5.0 g, 13.4 mmol) in CH₂Cl₂ (50 mL) was added dropwise. The reaction mixture was stirred at room temperature overnight. The solution was basified with aqueous saturated NaHCO₃ solution and extracted three times with CH₂Cl₂. The organic phase was dried (MgSO₄), filtered, and concentrated in vacuo. The resulting residue was purified by silica gel chromatography (80 g ISCO column) using 0-50% EtOAc/heptanes gradient to afford 4.67 g of product. 5-benzyloxy-1-(4-fluoro-3-methyl-phenyl)-2-isopropyl-indole-3-carbaldehyde (81%). ¹H NMR (300 MHz, Chloroform-d) δ 10.42 (s, 1H), 7.95 (d, J=2.5 Hz, 1H), 7.47-7.37 (m, 2H), 7.40-7.21 (m, 3H), 7.18-6.99 (m, 3H), 6.83 (dd, J=8.9, 2.5 Hz, 1H), 6.69 (dd, J=8.9, 0.5 Hz, 1H), 5.09 (s, 2H), 3.09 (p, J=7.2 Hz, 1H), 2.30 (d, J=2.0 Hz, 3H), 1.38 (dd, J=7.2, 2.1 Hz, 6H). ESI-MS m/z calc. 401.18, found 402.27 (M+1)⁺.

### Step 2. Synthesis of 2-(5-(benzyloxy)-1-(4-fluoro-3-methylphenyl)-2-isopropyl-1H-indol-3-yl)acetonitrile (C53)

To a cold (0° C.) solution of 5-benzyloxy-1-(4-fluoro-3-methyl-phenyl)-2-isopropyl-indole-3-carbaldehyde C₅₂ (1.75 g, 4.36 mmol) and TOSMIC (1.13 g, 5.67 mmol) in DME (16.5 mL) and EtOH (0.5 mL) was added potassium tert-butoxide (1.21 g, 10.46 mmol) portionwise. The reaction was stirred 1 hour at 0° C. MeOH (16.5 mL) was added and the reaction was heated to 90° C. and stirred for 30 minutes. The mixture was concentrated in vacuo. The residue was solubilized with an excess of aqueous saturated solution of NH₄Cl and CH₂Cl₂ to pH=4. The phases were separated and the aqueous phase was extracted twice with CH₂Cl₂. The combined organic phases were dried over Na₂SO₄, filtered and concentrated in vacuo. The resulting residue was purified by silica gel chromatography (40 g ISCO column) using 0-50% EtOAc/heptanes gradient to afford 1.12 g of product. 2-[5-benzyloxy-1-(4-fluoro-3-methyl-phenyl)-2-isopropyl-indol-3-yl]acetonitrile (62%). ¹H NMR (400 MHz, Chloroform-d) δ 7.53-7.45 (m, 2H), 7.44-7.35 (m, 2H), 7.35-7.28 (m, 1H), 7.20-7.04 (m, 4H), 6.86 (dd, J=8.8, 2.3 Hz, 1H), 6.79 (dd, J=8.9, 0.5 Hz, 1H), 5.14 (s, 2H), 3.90 (s, 2H), 3.00 (hept, J=7.3 Hz, 1H), 2.34 (d, J=2.0 Hz, 3H), 1.33 (d, J=4.1 Hz, 3H), 1.31 (d, J=4.1 Hz, 3H). ESI-MS m/z calc. 412.2, found 411.9 (M+1)⁺.

### Step 3. Synthesis of 2-(5-(benzyloxy)-1-(4-fluoro-3-methylphenyl)-2-isopropyl-1H-indol-3-yl)acetic acid (C54)

To a solution of 2-[5-benzyloxy-1-(4-fluoro-3-methyl-phenyl)-2-isopropyl-indol-3-yl]acetonitrile C53 (0.10 g, 0.24 mmol) in EtOH (1.7 mL) was added KOH (0.79 g of 50% w/w, 7.005 mmol) in water (1.7 mL). The mixture was irradiated in a microwave at 145° C. for 45 minutes. After cooling to room temperature, the reaction mixture was poured in a solution of water (20 mL) containing HCl (0.72 mL of 37% w/v, 7.276 mmol) and CH₂Cl₂ (20 mL). The aqueous phase was extracted twice with CH₂Cl₂. The combined organic layers were dried over Na₂SO₄, filtered and concentrated in vacuo to afford 104 mg of product. 2-[5-benzyloxy-1-(4-fluoro-3-methyl-phenyl)-2-isopropyl-indol-3-yl]acetic acid (97%). ESI-MS m/z calc. 431.19, found 432.45 (M+1)⁺. The crude product was used in the next step without further purification.

### Step 4: Synthesis of 2-(1-(4-fluoro-3-methylphenyl)-5-hydroxy-2-isopropyl-1H-indol-3-yl)acetic acid (153)

To a vial containing Pd on C (wet, Degussa, 0.027 g, 0.025 mmol) was added 2-[5-benzyloxy-1-(4-fluoro-3-methyl-phenyl)-2-isopropyl-indol-3-yl]acetic acid C54 (0.104 g, 0.241 mmol) The vial was sealed and was purged with one cycle of vacuum and nitrogen. EtOAc (4.8 mL) was added and The reaction mixture was evacuated and purged with hydrogen and stirred under a hydrogen atmosphere for 5 hours. The crude mixture was filtered through a pad of celite and the filtrate was concentrated in vacuo. The resulting residue was purified by silica gel chromatography (40 g ISCO column) using 0-30% MeOH/CH₂Cl₂ gradient to afford 33 mg of product. 2-[1-(4-fluoro-3-methyl-phenyl)-5-hydroxy-2-isopropyl-indol-3-yl]acetic acid (39%). ¹H NMR (400 MHz, Chloroform-d) δ 7.17-7.07 (m, 3H), 6.91-6.87 (m, 1H), 6.73 (d, J=8.6 Hz, 1H), 6.67 (d, J=8.7 Hz, 1H), 3.83 (s, 2H), 3.06-2.92 (m, 1H), 2.34 (s, 3H), 1.30-1.21 (m, 6H). ESI-MS m/z calc. 341.14, found 342.07 (M+1)⁺.

### Compounds 154 and 155

### Synthesis of 2-(1-(4-fluoro-3-methylphenyl)-5-hydroxy-2-isopropyl-1H-indol-3-yl)-2-methylpropanoic acid (154) and 2-(1-(4-fluoro-3-methylphenyl)-5-hydroxy-2-isopropyl-1H-indol-3-yl)propanoic acid (155)

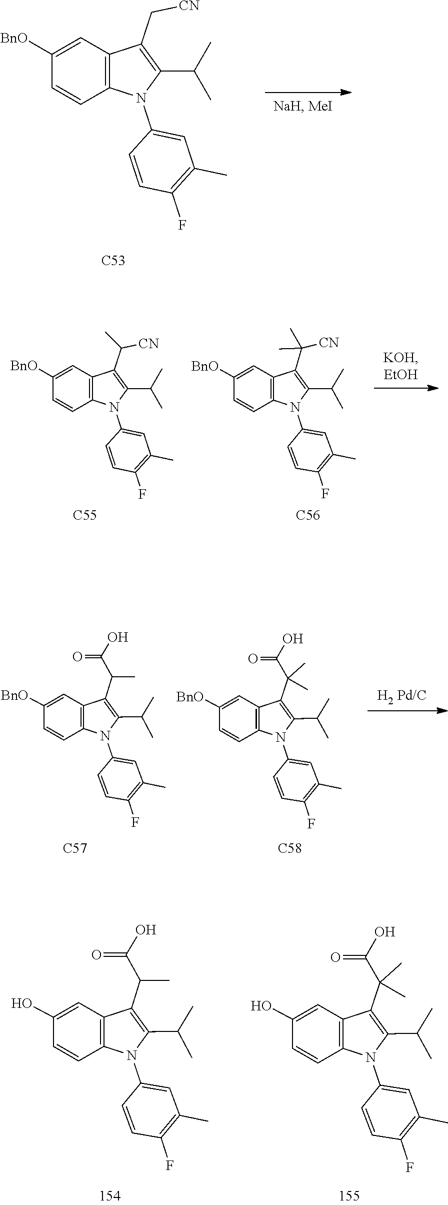

### Step 1. Synthesis of 2-(5-(benzyloxy)-1-(4-fluoro-3-methylphenyl)-2-isopropyl-1H-indol-3-yl)propanenitrile (C55) and 2-(5-(benzyloxy)-1-(4-fluoro-3-methylphenyl)-2-isopropyl-1H-indol-3-yl)-2-methylpropanenitrile (C56)

To a cold (0° C.) solution of 2-[5-benzyloxy-1-(4-fluoro-3-methyl-phenyl)-2-isopropyl-indol-3-yl]acetonitrile C53 (0.200 g, 0.485 mmol) in DMF (2.4 mL) was added sodium hydride (0.100 g, 2.425 mmol). The mixture was stirred until gas evolution stopped, then methyl iodide (0.151 mL, 2.426 mmol) was added. The reaction was stirred at 0° C. for 30 minutes and then 60 minutes at room temperature. The mixture was then stirred overnight at 50° C. The reaction was quenched by the addition of aqueous saturated NH₄Cl solution. The aqueous phase was extracted three times with CH₂Cl₂. The combined organic phases were dried (MgSO₄), filtered, and concentrated in vacuo. The resulting residue was purified by silica gel chromatography to afford an inseparable mixture of 2-[5-benzyloxy-1-(4-fluoro-3-methyl-phenyl)-2-isopropyl-indol-3-yl]-2-methyl-propanenitrile (85 mg, 23%): ESI-MS m/z calc. 440.2, found 439.9 (M+1)⁺ and 2-[5-benzyloxy-1-(4-fluoro-3-methyl-phenyl)-2-isopropyl-indol-3-yl]propanenitrile (56 mg, 27%): ESI-MS m/z calc. 426.21, found 427.81 (M+1)⁺. The mixture was taken on to the next step without further purification.

### Step 2. Synthesis of 2-(5-(benzyloxy)-1-(4-fluoro-3-methylphenyl)-2-isopropyl-1H-indol-3-yl)propanoic acid (C57) and 2-(5-(benzyloxy)-1-(4-fluoro-3-methylphenyl)-2-isopropyl-1H-indol-3-yl)-2-methylpropanoic acid (C58)

To a solution of 2-[5-benzyloxy-1-(4-fluoro-3-methyl-phenyl)-2-isopropyl-indol-3-yl]-2-methyl-propanenitrile C55 (0.085 g, 0.190 mmol) and 2-[5-benzyloxy-1-(4-fluoro-3-methyl-phenyl)-2-isopropyl-indol-3-yl]propanenitrile C56 (0.056 g, 0.131 mmol) in EtOH (3.2 mL) was added a solution of KOH (0.625 g of 50% w/w, 5.570 mmol) in water (3.2 mL). The reaction mixture was irradiated in a microwave reactor at 180° C. for 2.5 hours. After cooling to room temperature, the reaction mixture was poured in a solution of water (20 mL) containing HCl (0.570 mL of 37% w/v, 5.784 mmol) and CH₂Cl₂ (20 mL). The phases were separated and the aqueous phase was extracted twice with CH₂Cl₂. The combined organic layers were dried over Na₂SO₄, filtered and concentrated in vacuo to afford a mixture of 2-[5-benzyloxy-1-(4-fluoro-3-methyl-phenyl)-2-isopropyl-indol-3-yl]-2-methyl-propanamide (71 mg, 80%): ESI-MS m/z calc. 458.24, found 459.41 (M+1)⁺ and 2-[5-benzyloxy-1-(4-fluoro-3-methyl-phenyl)-2-isopropyl-indol-3-yl]propanoic acid (71 mg, 83%): ESI-MS m/z calc. 445.21, found 446.40 (M+1)⁺. The mixture was used in the next step without further purification.

### Step 3: Synthesis of 2-(1-(4-fluoro-3-methylphenyl)-5-hydroxy-2-isopropyl-1H-indol-3-yl)-2-methylpropanoic acid (154) and 2-(1-(4-fluoro-3-methylphenyl)-5-hydroxy-2-isopropyl-1H-indol-3-yl)propanoic acid (155)

To a vial containing Pd on C (wet, Degussa, 0.027 g, 0.025 mmol) was added 2-[5-benzyloxy-1-(4-fluoro-3-methyl-phenyl)-2-isopropyl-indol-3-yl]acetic acid (0.104 g, 0.241 mmol). The vial was sealed and was purged with one cycle of vacuum and nitrogen. EtOAc (3 mL) was added and The reaction mixture was evacuated and purged with hydrogen and stirred under a hydrogen atmosphere for 16 hours. The crude mixture was filtered through a pad of celite and the filtrate was concentrated in vacuo. The crude residue was purified by reverse phase flash chromatography (RF ISCO, C18 column, 30 g) eluting with CH₃CN/water (0-100%, 0.1% TFA) to afford 2-[1-(4-fluoro-3-methyl-phenyl)-5-hydroxy-2-isopropyl-indol-3-yl]propanoic acid (4.9 mg, 9%): ¹H NMR (400 MHz, Chloroform-d) δ 7.19-7.00 (m, 4H), 6.72 (d, J=8.6 Hz, 1H), 6.68-6.60 (m, 1H), 4.27-4.14 (m, 1H), 3.07-2.92 (m, 1H), 2.33 (s, 3H), 1.63 (d, J=7.1 Hz, 3H), 1.34 (d, J=7.2 Hz, 3H), 1.29 (d, J=7.0 Hz, 3H). ESI-MS m/z calc. 355.1584, found 356.07 (M+1)⁺ and 2-[1-(4-fluoro-3-methyl-phenyl)-5-hydroxy-2-isopropyl-indol-3-yl]-2-methyl-propanamide (20.3 mg, 35%): ¹H NMR (400 MHz, Chloroform-d) δ 7.35-7.30 (m, 1H), 7.19-7.10 (m, 3H), 6.63 (d, J=8.8 Hz, 1H), 6.54 (d, J=8.7 Hz, 1H), 5.77 (s, 1H), 5.47 (s, 1H), 3.31 (s, 1H), 2.33 (s, 3H), 1.81 (s, 6H), 1.07 (d, J=7.1 Hz, 6H). ESI-MS m/z calc. 368.19, found 369.13 (M+1)⁺.

### Preparation 156

### Synthesis of 2-(2-cyclopropyl-1-(4-fluoro-3-methylphenyl)-5-hydroxy-1H-indol-3-yl)-3-phenylpropanoic acid (156)

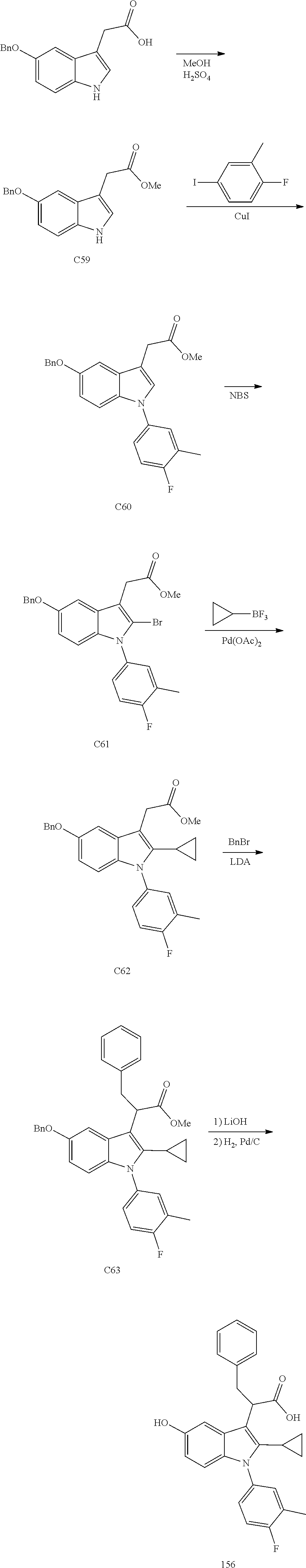

### Step 1. Synthesis of methyl 2-(5-(benzyloxy)-1H-indol-3-yl)acetate (C59)

To a solution of 2-(5-benzyloxy-1H-indol-3-yl)acetic acid (10.0 g, 35.6 mmol) in MeOH (50.0 mL, 1.2 mol) was added H₂SO₄ (2.0 mL, 37.5 mmol). The reaction mixture was heated to reflux and stirred for 3 hours and then cooled to room temperature. The solvent was evaporated under reduced pressure. The residue was dissolved in EtOAc (200 mL) and washed with aqueous saturated NaHCO₃ solution. The organic phase was dried (MgSO₄), filtered, and concentrated in vacuo. The resulting residue was purified by silica gel chromatography using 10-90% EtOAc/heptanes gradient to afford 10.1 g of product. Methyl 2-(5-benzyloxy-1H-indol-3-yl)acetate (96%). ¹H NMR (400 MHz, Chloroform-d) δ 7.98 (s, 1H), 7.51 (ddd, J=6.8, 1.5, 0.8 Hz, 2H), 7.46-7.39 (m, 2H), 7.35 (d, J=7.3 Hz, 1H), 7.30-7.25 (m, 2H), 7.18 (dd, J=3.9, 2.5 Hz, 2H), 6.98 (dd, J=8.8, 2.4 Hz, 1H), 5.14 (s, 2H), 3.76 (s, 2H), 3.71 (s, 3H). ESI-MS m/z calc. 295.12, found 296.09 (M+1)⁺.

### Step 2. Synthesis of methyl 2-(5-(benzyloxy)-1-(4-fluoro-3-methylphenyl)-1H-indol-3-yl)acetate (C60)

Copper (I) iodide (3.20 g, 16.80 mmol) was added to nitrogen purged solution of methyl 2-(5-benzyloxy-1H-indol-3-yl)acetate C59 (10.50 g, 34.12 mmol), 1-fluoro-4-iodo-2-methyl-benzene (10.50 g, 44.49 mmol), KH₂PO₄ (9.30 g, 68.34 mmol) and N,N-dimethylethylenediamine (3.60 mL, 33.81 mmol) in toluene (80 mL) and DMSO (9 mL). The solution was heated at 120° C. for 20 h. The reaction mixture was cooled to room temperature and filtered. The solid was washed with EtOAc (200 mL). The filtrate was washed with aqueous saturated NaHCO₃ solution. The organic phase was dried over Na₂SO₄, filtered and concentrated in vacuo. The resulting residue was purified by silica gel chromatography (120 g ISCO column) using 0-60% EtOAc/heptanes gradient to afford 6.4 g of product. Methyl 2-[5-benzyloxy-1-(4-fluoro-3-methyl-phenyl)indol-3-yl]acetate (45%). ¹H NMR (400 MHz, Chloroform-d) δ 7.59-7.48 (m, 2H), 7.44-7.33 (m, 4H), 7.32-7.24 (m, 4H), 7.20 (d, J=2.4 Hz, 1H), 7.14 (t, J=8.8 Hz, 1H), 6.99 (dd, J=9.0, 2.5 Hz, 1H), 5.16 (s, 2H), 3.80 (d, J=0.9 Hz, 2H), 3.73 (s, 3H), 2.37 (d, J=2.0 Hz, 3H). ESI-MS m/z calc. 403.16, found 404.1 (M+1)⁺.

### Step 3. Synthesis of methyl 2-(5-(benzyloxy)-2-bromo-1-(4-fluoro-3-methylphenyl)-1H-indol-3-yl)acetate (C61)

To solution of methyl 2-[5-benzyloxy-1-(4-fluoro-3-methyl-phenyl)indol-3-yl]acetate C60 (0.81 g, 1.94 mmol) in CCl₄ (15 mL) was added N-bromosuccinimide. The reaction mixture was stirred at room temperature for 3 hours. The solvent was evaporated under reduced pressure. The resulting residue was purified by silica gel chromatography (40 g ISCO column) using 0-60% EtOAc/heptanes gradient to afford 0.32 g of product. methyl 2-[5-benzyloxy-2-bromo-1-(4-fluoro-3-methyl-phenyl)indol-3-yl]acetate (30%). ¹H NMR (400 MHz, Chloroform-d) δ 7.48-7.40 (m, 2H), 7.38-7.31 (m, 2H), 7.30-7.24 (m, 1H), 7.18-7.04 (m, 4H), 6.94 (d, J=8.9 Hz, 1H), 6.85 (dd, J=8.9, 2.4 Hz, 1H), 5.08 (s, 2H), 3.76 (s, 2H), 3.67 (s, 3H), 2.31 (d, J=2.0 Hz, 3H). ESI-MS m/z calc. 481.06888, found 482.0 (M+1)⁺.

### Step 4. Synthesis of methyl 2-(5-(benzyloxy)-2-cyclopropyl-1-(4-fluoro-3-methylphenyl)-1H-indol-3-yl)acetate (C62)

Palladium (II) acetate (0.114 g, 0.508 mmol) was added to a nitrogen purged solution of cyclopropyl(trifluoro)boranuide (Potassium Ion (1)) (1.90 g, 12.84 mmol), methyl 2-[5-benzyloxy-2-bromo-1-(4-fluoro-3-methyl-phenyl)indol-3-yl]acetate C61 (1.40 g, 2.55 mmol) and X-Phos (1.89 g, 2.546 mmol) and Pd(OAc)₂ (0.114 g, 0.507 mmol) in toluene (70 mL) and water (10 mL). The reaction was capped in a sealable tube (Qian Cap) and the reaction mixture was heated at 120° C. for 18 hours. Additional cyclopropyl(trifluoro)-boranuide (Potassium Ion (1)) (1.90 g, 12.84 mmol), X-Phos (1.89 g, 2.55 mmol) and Pd(OAc)₂ (0.114 g, 0.507 mmol) were added and the reaction was heated at 120° C. for 18 hours. The mixture was cooled to room temperature and the solid was filtered. The solid was washed with EtOAc (100 mL). The combined filtrate was washed with water (50 mL) and the organic phase was separated. The organic layer was dried (MgSO₄) and the solvent was evaporated under reduced pressure. The resulting residue was purified by silica gel chromatography (40 g ISCO column) using 0-60% EtOAc/heptanes gradient to afford 0.72 g of product. The crude residue was purified by reverse phase flash chromatography (RF ISCO, C18 column, 30 g) eluting with CH₃CN/water (0-100%, 0.1% TFA) to afford. The crude residue was purified again by reverse phase flash chromatography (RF ISCO, C18 column, 30 g) eluting with CH₃CN/water (0-100%, 0.1% TFA) to afford 650 mg of product. methyl 2-[5-benzyloxy-2-cyclopropyl-1-(4-fluoro-3-methyl-phenyl)indol-3-yl]acetate (57%). 1H NMR (400 MHz, Chloroform-d) δ 7.52-7.49 (m, 2H), 7.44-7.38 (m, 2H), 7.36-7.24 (m, 1H), 7.24-7.13 (m, 4H), 7.01 (dd, J=8.9, 0.5 Hz, 1H), 6.88 (dd, J=8.8, 2.4 Hz, 1H), 5.14 (s, 2H), 3.71 (s, 3H), 2.36 (d, J=2.0 Hz, 3H), 1.79-1.76 (m, 1H), 0.85-0.69 (m, 2H), 0.63-0.40 (m, 2H). ESI-MS m/z calc. 443.2, found 444.2 (M+1)⁺.

### Step 5. Synthesis of methyl 2-(5-(benzyloxy)-2-cyclopropyl-1-(4-fluoro-3-methylphenyl)-1H-indol-3-yl)-3-phenylpropanoate (C63)

To a cold (−78° C.) solution of methyl 2-[5-benzyloxy-2-cyclopropyl-1-(4-fluoro-3-methyl-phenyl)indol-3-yl]acetate (C62) (0.27 g, 0.60 mmol) in anhydrous THF (10 mL) was added LDA (450 μL of 2 M, 0.9000 mmol). The solution was stirred at −78° C. for 45 minutes. A solution of benzyl bromide (1.10 mL, 9.29 mmol) in THE (1 mL) was added dropwise and the reaction was stirred at −78° C. for 2 hours and slowly warmed to room temperature. The reaction was quenched with aqueous saturated NH₄Cl solution (5 mL) and extracted twice with EtOAc (10 mL). The combined organic phases were dried over Na₂SO₄, filtered and concentrated in vacuo. The resulting residue was purified by silica gel chromatography (40 g ISCO column) using 0-5% EtOAc/heptanes gradient to afford 240 mg of product. methyl 2-[5-benzyloxy-2-cyclopropyl-1-(4-fluoro-3-methyl-phenyl)indol-3-yl]-3-phenyl-propanoate (74%). ¹H NMR (400 MHz, Chloroform-d) δ 7.53-7.44 (m, 2H), 7.35-7.30 (m, 3H), 7.29-6.90 (m, 10H), 6.81 (dd, J=8.8, 2.4 Hz, 1H), 5.11 (s, 2H), 4.32 (dd, J=9.1, 6.5 Hz, 1H), 3.58 (s, 3H), 3.48 (dd, J=13.3, 6.5 Hz, 1H), 3.10 (dd, J=13.3, 9.1 Hz, 1H), 2.26 (s, 3H), 1.11-0.90 (m, 1H), 0.62-0.60 (m, 1H), 0.51-0.47 (m, 1H), 0.43-0.26 (m, 1H), 0.04-0.05 (m, 1H). ESI-MS m/z calc. 533.2, found 534.2 (M+1)⁺.

### Step 6. Synthesis of 2-(2-cyclopropyl-1-(4-fluoro-3-methylphenyl)-5-hydroxy-1H-indol-3-yl)-3-phenylpropanoic acid (156)

To a stirred solution of methyl 2-[5-benzyloxy-2-cyclopropyl-1-(4-fluoro-3-methyl-phenyl)indol-3-yl]-3-phenyl-propanoate C63 (0.075 g, 0.127 mmol) in THE (1 mL), MeOH (3 mL) and water (1 mL) was added LiOH (0.050 g, 2.088 mmol). The reaction mixture was stirred at room temperature for 18 hours and the solvent was removed under reduced pressure. The residue was dissolved in water (2 mL) and acidified with 6 N HCl. The white ppt was extracted with EtOAc (3×5 mL). The combined organic extracts were dried and concentrated under reduced pressure to afford 65 mg of product. 2-[5-benzyloxy-2-cyclopropyl-1-(4-fluoro-3-methyl-phenyl)indol-3-yl]-3-phenyl-propanoic acid (96%). ESI-MS m/z calc. 519.22, found 520.25 (M+1)⁺.

To a solution of 2-[5-benzyloxy-2-cyclopropyl-1-(4-fluoro-3-methyl-phenyl)indol-3-yl]-3-phenyl-propanoic acid (0.060 mg, 0.112 mmol) in MeOH (5 mL) and EtOAc (2 mL) was added Pd/C (0.100 g, 0.094 mmol). The mixture was purged with nitrogen. The reaction mixture was evacuated and purged with hydrogen and stirred under a hydrogen atmosphere for 1 hour. The crude mixture was filtered through a pad of celite and the filtrate was concentrated in vacuo. The crude residue was purified by reverse phase flash chromatography (RF ISCO, C18 column, 30 g) eluting with CH₃CN/water (0-100%, 0.1% TFA) to afford 35 mg of product. 2-[2-cyclopropyl-1-(4-fluoro-3-methyl-phenyl)-5-hydroxy-indol-3-yl]-3-phenyl-propanoic acid (70%). ¹H NMR (400 MHz, Chloroform-d) δ 7.27-7.25 (m, 2H), 7.21-7.12 (m, 3H), 7.07 (t, J=8.8 Hz, 1H), 6.95.6.93 (m, 4H), 6.75 (dd, J=8.8, 2.3 Hz, 1H), 4.38 (dd, J=9.5, 5.8 Hz, 1H), 3.49-3.44 (m, 1H), 3.15-2.90 (m, 1H), 2.30 (s, 3H), 1.11-1.08 (m, 1H), 0.64-0.62 (m, 1H), 0.55-0.23 (m, 2H). ESI-MS m/z calc. 429.2, found 430.2 (M+1)⁺.

TABLE 12
Method of preparation, structure and physicochemical data for compounds 157-162

| Compound | Product | ¹H NMR; LCMS m/z [M + H]⁺ |
|---|---|---|
| 157 | 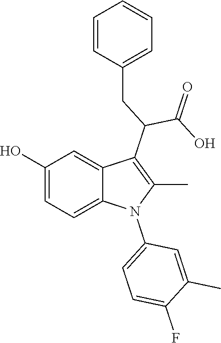 [SMILES: Cc1cc(-n2c(C)c(C(Cc3ccccc3)C(=O)O)c3cc(O)ccc32)ccc1F] | ¹H NMR (400 MHz, Chloroform- d) δ 7.23-7.08 (m, 3H), 7.04 (d, J = 2.2 Hz, 1H), 6.75-6.52 (m, 2H), 3.48 (d, J = 14.9 Hz, 1H), 3.31 (p, J = 7.3 Hz, 1H), 3.17 (d, J = 14.9 Hz, 1H), 2.34 (d, J = 2.1 Hz, 3H), 1.64 (s, 3H), 1.16 (t, J = 6.5 Hz, 6H). LCMS m/z 386.3 [M + H]⁺. |
| 158¹ | 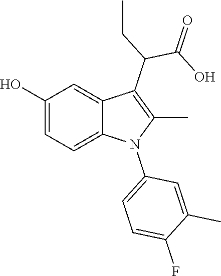 [SMILES: CCC(C(=O)O)c1c(C)n(-c2ccc(F)c(C)c2)c2ccc(O)cc12] | ¹H NMR (400 MHz, Chloroform- d) δ 7.18-7.07 (m, 4H), 6.89 (d, J = 8.7 Hz, 1H), 6.67 (dd, J = 8.7, 2.4 Hz, 1H), 3.78-3.64 (m, 1H), 2.36 (d, J = 2.0 Hz, 3H), 2.33- 2.28 (m, 1H), 2.25 (s, 3H), 2.07- 1.84 (m, 1H), 0.95 (t, J = 7.4 Hz, 3H). LCMS m/z 342.2 [M + H]⁺. |
| 159¹ | 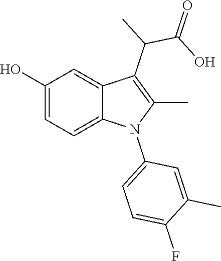 [SMILES: Cc1cc(-n2c(C)c(C(C)C(=O)O)c3cc(O)ccc32)ccc1F] | ¹H NMR (400 MHz, Chloroform- d) δ 7.18-7.09 (m, 4H), 6.89 (d, J = 8.9 Hz, 1H), 6.68 (dd, J = 8.7, 2.4 Hz, 1H), 4.01 (q, J = 7.2 Hz, 1H), 2.35 (d, J = 2.0 Hz, 3H), 2.25 (s, 3H), 1.63 (d, J = 7.2 Hz, 3H). LCMS m/z 328.1 [M + H]⁺. |
| 160 | 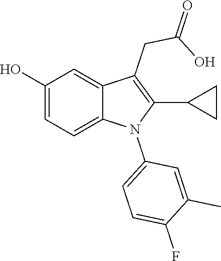 [SMILES: Cc1cc(-n2c(C3CC3)c(CC(=O)O)c3cc(O)ccc32)ccc1F] | ¹H NMR (400 MHz, Chloroform- d) δ 7.24-7.10 (m, 4H), 7.01- 6.87 (m, 2H), 6.72 (dd, J = 8.7, 2.5 Hz, 1H), 3.91 (s, 3H), 2.36 (d, J = 1.9 Hz, 3H), 1.81-1.79 (m, 1H)), 0.80-0.73 (m, 2H), 0.57- 0.47 (m, 2H). LCMS m/z 340.1 [M + H]⁺. |
| 161¹ | 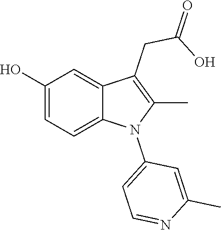 [SMILES: Cc1cc(-n2c(C)c(CC(=O)O)c3cc(O)ccc32)ccn1] | ¹H NMR (400 MHz, DMSO-d6) δ 8.56 (d, J = 5.4 Hz, 1H), 8.39 (s, 1H), 7.32 (d, J = 2.0 Hz, 1H), 7.24 (dd, J = 5.4, 1.7 Hz, 1H), 7.04 (d, J = 8.7 Hz, 1H), 6.84 (d, J = 2.2 Hz, 1H), 6.57 (dd, J = 8.7, 2.3 Hz, 1H), 3.51 (s, 2H), 2.54 (s, 3H), 2.25 (s, 3H). LCMS m/z 297.1 [M + H]⁺. |
| 162¹ | 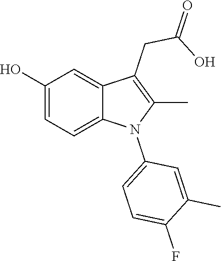 [SMILES: Cc1cc(-n2c(C)c(CC(=O)O)c3cc(O)ccc32)ccc1F] | ¹H NMR (400 MHz, Acetone-d6) δ 7.28 (d, J = 8.2 Hz, 1H), 7.26- 7.17 (m, 2H), 6.99 (d, J = 2.0 Hz, 1H), 6.84 (d, J = 8.7 Hz, 1H), 6.63 (dd, J = 8.7, 2.1 Hz, 1H), 3.67 (s, 1H), 2.36 (d, J = 1.3 Hz, 3H), 2.23 (s, 3H). LCMS m/z 314.0 [M + H]⁺. |

¹methyl 2-(5-methoxy-2-methyl-1H-indol-3-yl)acetate was used as an alternative to C62. The hydrogenation step was omitted. BBr3 was used in the final step to remove the OMe group.

### Compound 163

### Synthesis of 3-(6-fluoro-1-(4-fluoro-3-methylphenyl)-5-hydroxy-2-isopropyl-1H-indol-3-yl)propanoic acid (163)

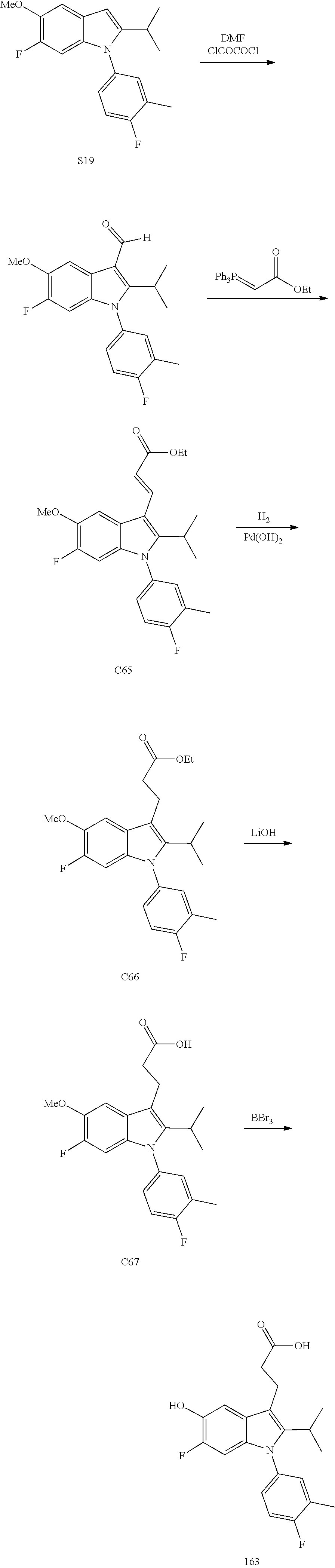

### Step 1. Synthesis of 6-fluoro-1-(4-fluoro-3-methylphenyl)-2-isopropyl-5-methoxy-1H-indole-3-carbaldehyde (C64)

To a cold (0° C.) solution of DMF (3.00 mL, 38.74 mmol) in CH₂Cl₂ (5 mL) was added oxalyl chloride (3.3 mL of 2 M, 6.600 mmol). The solution was stirred at room temperature for 30 minutes. Added a solution of 6-fluoro-1-(4-fluoro-3-methyl-phenyl)-2-isopropyl-5-methoxy-indole S19 (1.20 g, 3.67 mmol) in CH₂Cl₂ (15 mL). The resulting solution was stirred at room temperature for 2 hours. A solution of aqueous saturated NaHCO₃ was added slowly to quench. The organic phase was dried (MgSO₄), filtered, and concentrated in vacuo The resulting residue was purified by silica gel chromatography using 0-60% EtOAc/heptanes gradient to afford 1.12 g of product. 6-fluoro-1-(4-fluoro-3-methyl-phenyl)-2-isopropyl-5-methoxy-indole-3-carbaldehyde (89%). ¹H NMR (400 MHz, Chloroform-d) δ 10.51 (s, 1H), 8.01 (d, J=8.4 Hz, 1H), 7.30-7.20 (m, 2H), 7.20-7.04 (m, 2H), 6.62 (d, J=11.0 Hz, 1H), 4.01 (s, 3H), 3.19 (p, J=7.2 Hz, 1H), 2.40 (d, J=2.0 Hz, 3H), 1.47 (dd, J=7.2, 2.6 Hz, 6H). ESI-MS m/z calc. 343.1384, found 344.19 (M+1)⁺.

### Step 2. Synthesis of ethyl (E)-3-(6-fluoro-1-(4-fluoro-3-methylphenyl)-2-isopropyl-5-methoxy-1H-indol-3-yl)acrylate (C65)

To a solution of 6-fluoro-1-(4-fluoro-3-methyl-phenyl)-2-isopropyl-5-methoxy-indole-3-carbaldehyde C64 (0.36 g, 1.05 mmol) in toluene (10 mL) was added ethyl 2-(triphenyl-Î>>5-phosphanylidene)acetate (0.73 g, 2.10 mmol). The reaction mixture was heated at 120° C. for 48 hours. The mixture was cooled to room temperature and diluted with water. The organic phase was dried (MgSO₄), filtered, and concentrated in vacuo. The resulting residue was purified by silica gel chromatography using 0-60% EtOAc/heptanes gradient to afford 0.24 g of product. Ethyl (E)-3-[6-fluoro-1-(4-fluoro-3-methyl-phenyl)-2-isopropyl-5-methoxy-indol-3-yl]prop-2-enoate (55%). 1H NMR (400 MHz, Chloroform-d) δ 8.22 (d, J=15.8 Hz, 1H), 7.43 (d, J=8.0 Hz, 1H), 7.26-7.06 (m, 3H), 6.63 (d, J=11.2 Hz, 1H), 6.38 (d, J=15.9 Hz, 1H), 4.33 (q, J=7.1 Hz, 2H), 4.00 (s, 3H), 3.16 (p, J=7.2 Hz, 1H), 2.39 (d, J=2.0 Hz, 3H), 1.48-1.32 (m, 9H). ESI-MS m/z calc. 413.18, found 414.28 (M+1)⁺.

### Step 3. Synthesis of ethyl 3-(6-fluoro-1-(4-fluoro-3-methylphenyl)-2-isopropyl-5-methoxy-1H-indol-3-yl)propanoate (C66)

To a solution of ethyl (E)-3-[6-fluoro-1-(4-fluoro-3-methyl-phenyl)-2-isopropyl-5-methoxy-indol-3-yl]prop-2-enoate C65 (0.24 g, 0.57 mmol) in MeOH (10 mL) purged with nitrogen was added palladium hydroxide (0.05 g, 0.07 mmol). The reaction mixture was evacuated and purged with hydrogen and stirred under a hydrogen atmosphere for 2 hours. The crude mixture was filtered through a pad of celite and the filtrate was concentrated in vacuo to afford 220 mg of product. Ethyl 3-[6-fluoro-1-(4-fluoro-3-methyl-phenyl)-2-isopropyl-5-methoxy-indol-3-yl]propanoate (92%). ¹H NMR (400 MHz, Chloroform-d) δ 7.23-7.04 (m, 4H), 6.62 (d, J=11.5 Hz, 1H), 4.21 (q, J=7.1 Hz, 2H), 3.97 (s, 3H), 3.26-3.15 (m, 2H), 3.08-2.97 (m, 1H), 2.74-2.61 (m, 2H), 2.36 (d, J=2.0 Hz, 3H), 1.38-1.22 (m, 9H). ESI-MS m/z calc. 415.19, found 416.35 (M+1)⁺.

### Step 4. Synthesis of 3-(6-fluoro-1-(4-fluoro-3-methylphenyl)-2-isopropyl-5-methoxy-1H-indol-3-yl)propanoic acid (C67)

To a solution of ethyl 3-[6-fluoro-1-(4-fluoro-3-methyl-phenyl)-2-isopropyl-5-methoxy-indol-3-yl]propanoate C66 (0.14 g, 0.34 mmol) in MeOH (6.0 mL), THE (3.0 mL) and water (1.0 mL) was added lithium hydroxide (0.14 g, 3.36 mmol). After 2 hours, the solvent was concentrated in vacuo and the crude residue was dissolved in water (10 mL) and acidified with 10% HCl. The aqueous phase was extracted three times with EtOAc. The organic phase was dried (MgSO₄), filtered, and concentrated in vacuo to afford 130 mg of product. 3-[6-Fluoro-1-(4-fluoro-3-methyl-phenyl)-2-isopropyl-5-methoxy-indol-3-yl]propanoic acid (99%). ¹H NMR (400 MHz, Chloroform-d) δ 7.21-6.98 (m, 4H), 6.63 (d, J=11.5 Hz, 1H), 3.97 (s, 3H), 3.30-3.18 (m, 2H), 3.11-2.89 (m, 1H), 2.82-2.62 (m, 2H), 2.37 (d, J=2.0 Hz, 1H), 1.30 (d, J=2.4 Hz, 3H), 1.28 (d, J=2.5 Hz, 3H). ESI-MS m/z calc. 387.16, found 388.26 (M+1)⁺.

### Step 5. Synthesis of 3-(6-fluoro-1-(4-fluoro-3-methylphenyl)-5-hydroxy-2-isopropyl-1H-indol-3-yl)propanoic acid (163)

To a cold (0° C.) solution of 3-[6-fluoro-1-(4-fluoro-3-methyl-phenyl)-2-isopropyl-5-methoxy-indol-3-yl]propanoic acid C67 (0.130 g, 0.334 mmol) in CH₂Cl₂ (5.0 mL) was added tribromoborane (1.0 mL of 1 M, 1.000 mmol). The reaction mixture was stirred at room temperature for 3 hours. The mixture was diluted into water and extracted with CH₂Cl₂. The organic phase was dried (MgSO₄), filtered, and concentrated in vacuo. The crude residue was purified by reverse phase flash chromatography (RF ISCO, C₁₈ column, 30 g) eluting with CH₃CN/water (0-100%, 0.1% TFA) to afford 112 mg of product. 3-[6-fluoro-1-(4-fluoro-3-methyl-phenyl)-5-hydroxy-2-isopropyl-indol-3-yl]propanoic acid (86%). ¹H NMR (400 MHz, DMSO-d6) δ 12.19 (s, 1H), 9.09 (s, 1H), 7.32-7.28 (m, 2H), 7.19 (d, J=4.2 Hz, 1H), 7.00 (d, J=8.5 Hz, 1H), 6.49 (d, J=11.5 Hz, 1H), 5.75 (s, 1H), 3.00-2.96 (m, 4H), 2.30 (s, 3H), 1.21 (d, J=7.2 Hz, 6H). MS m/z calc. 373.14896, found 374.29 (M+1)⁺.

### Compound 164

### Synthesis of 4-(1-(4-fluorophenyl)-5-hydroxy-2-(1-methoxy-2-methylpropan-2-yl)-1H-indol-3-yl)benzoic acid (164)

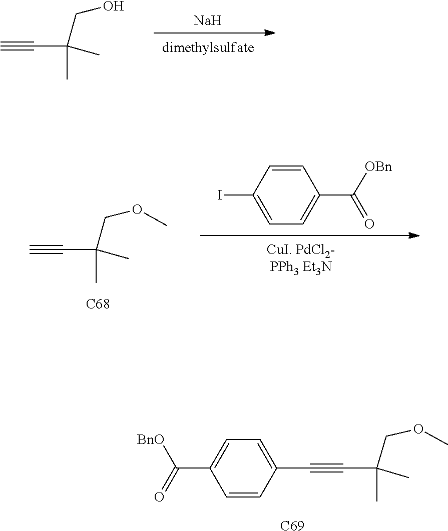

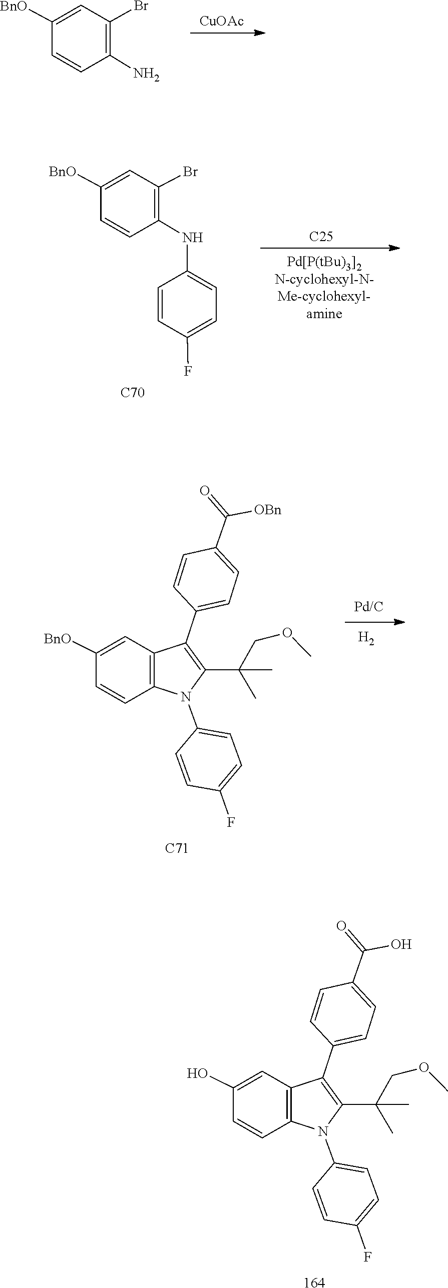

### Step 1. Synthesis of 4-methoxy-3,3-dimethylbut-1-yne (C68)

To a cold (0° C.) solution of 2,2-dimethylbut-3-yn-1-ol (20.0 g, 203.8 mmol) in DMF (140 mL) was added NaH (8.2 g of 60% w/w, 204.0 mmol) portion wise over 10 minutes. The mixture was stirred for 30 minutes. To the mixture was added dropwise dimethyl sulfate (23.5 mL, 248.4 mmol). After 10 minutes at 0° C., the reaction was stirred at room temperature for 90 minutes. The mixture was diluted into 280 mL of cold water and stirred for 15 minutes. The organic phase was filtered to afford 16 g of crude product that was used without further purification. 4-methoxy-3,3-dimethyl-but-1-yne (73%). ¹H NMR (300 MHz, Chloroform-d) δ 3.43 (s, 3H), 3.27 (s, 2H), 2.15 (s, 1H), 1.25 (s, 6H).

### Step 2. Synthesis of benzyl 4-(4-methoxy-3,3-dimethylbut-1-yn-1-yl)benzoate (C69)

A solution of benzyl 4-iodobenzoate (15.00 g, 44.40 mmol), Pd(PPh₃)₂Cl₂ (0.94 g, 1.33 mmol), iodocopper (0.51 g, 2.66 mmol) in triethylamine (100 mL) a n d THE (100 mL) was purged with nitrogen for 5 minutes. To the mixture was added 4-methoxy-3,3-dimethyl-but-1-yne C68 (7.22 g, 64.37 mmol). The mixture was purged with nitrogen for 1 minute. The flask was stirred at room temperature for 5 minutes and then heated to 50° C. for 2 hours. The mixture was filtered and resulting solid was washed twice with EtOAc. The filtrate was concentrated in vacuo. The resulting residue was purified by silica gel chromatography (330 g ISCO column) using 0-50% EtOAc/heptanes gradient to afford 13 g of product. Benzyl 4-(4-methoxy-3,3-dimethyl-but-1-ynyl)benzoate (79%). ¹H NMR (300 MHz, Chloroform-d) δ 8.04-7.94 (m, 2H), 7.51-7.32 (m, 7H), 5.37 (s, 2H), 3.46 (q, 3H), 3.36 (q, 2H), 1.33 (s, 6H). ESI-MS m/z calc. 322.2, found 323.1 (M+1)⁺.

### Step 3. Synthesis of 4-(benzyloxy)-2-bromo-N-(4-fluorophenyl)aniline (C70)

A 50 mL round bottom flask charged with 4-benzyloxy-2-bromo-aniline (0.50 g, 1.78 mmol), (4-fluorophenyl)boronic acid (0.50 g, 3.55 mmol), copper(II)acetate (0.65 g, 3.55 mmol) and 4 A Sieves (0.50 g) in CH₂Cl₂ (15 mL) was stirred open to the air for 15 minutes. Triethylamine 0.62 mL, 4.45 mmol) was added dropwise at ambient temperature and the resulting dark blue/purple mixture was stirred open to the air for 16 hours. The crude reaction mixture was diluted with ethyl acetate, then washed with water and brine. The combined organic phases were washed with brine, dried (MgSO₄), filtered, and concentrated in vacuo. The resulting residue was purified by silica gel chromatography (40 g ISCO column) using 0-20% EtOAc/heptanes gradient to afford 380 mg of product. 4-benzyloxy-2-bromo-N-(4-fluorophenyl)aniline (56%). ¹H NMR (400 MHz, DMSO-d6) δ 7.47-7.37 (m, 5H), 7.37-7.34 (m, 1H), 7.32 (d, J=2.9 Hz, 1H), 7.16 (d, J=8.8 Hz, 1H), 7.00 (ddd, J=8.8, 5.9, 3.1 Hz, 3H), 6.81-6.74 (m, 2H), 5.09 (s, 2H). ESI-MS m/z calc. 371.03, found 372.19 (M+1)⁺.

### Step 4. Synthesis of benzyl 4-(5-(benzyloxy)-1-(4-fluorophenyl)-2-(I-methoxy-2-methylpropan-2-yl)-1H-indol-3-yl)benzoate (C71)

A solution of 4-benzyloxy-2-bromo-N-(4-fluorophenyl)aniline C70 (0.25 g, 0.66 mmol), benzyl 4-(4-methoxy-3,3-dimethyl-but-1-ynyl)benzoate C69 (0.38 g, 1.05 mmol) and Pd[P(tBu)₃]₂ (0.017 g, 0.033 mmol) was evacuated and purged with nitrogen twice. A solution of 1,4-dioxane (4 mL) and N-cyclohexyl-N-methyl-cyclohexanamine (0.35 mL, 1.61 mmol) was bubbled with nitrogen for 2 minutes and subsequently added to the reaction vial. The reaction vial was sealed and heated to 100° C. LCMS after 1 hour shows complete consumption of limiting starting material. The reaction solution was cooled to room temperature, diluted with water, and extracted with ethyl acetate. The organic phase was washed with brine, dried (MgSO₄), filtered, and concentrated in vacuo. The resulting residue was purified by silica gel chromatography (40 g ISCO column) using 10-35% EtOAc/heptanes gradient to afford 355 mg of product. Benzyl 4-[5-benzyloxy-1-(4-fluorophenyl)-2-(2-methoxy-1,1-dimethyl-ethyl)indol-3-yl]benzoate (79%). ¹H NMR (400 MHz, Chloroform-d) δ 8.21-8.14 (m, 2H), 7.59-7.49 (m, 4H), 7.50-7.29 (m, 10H), 7.28-7.18 (m, 2H), 6.82 (dd, J=8.8, 2.4 Hz, 1H), 6.63-6.53 (m, 2H), 5.45 (s, 2H), 4.93 (s, 2H), 3.08 (s, 3H), 3.07 (s, 2H), 1.12 (s, 6H). ESI-MS m/z calc. 613.26, found 614.37 (M+1)⁺.

### Step 5. Synthesis of benzyl 4-(5-(benzyloxy)-1-(4-fluorophenyl)-2-(I-methoxy-2-methylpropan-2-yl)-1H-indol-3-yl)benzoate (164)

To a slurry of Pd/C (0.06 g, 0.06 mmol) in EtOH (10 mL) was added a solution of benzyl 4-[5-benzyloxy-1-(4-fluorophenyl)-2-(2-methoxy-1,1-dimethyl-ethyl)indol-3-yl]benzoate C71 (0.14 g, 0.21 mmol) in EtOAc (10 mL). The reaction vial was evacuated and backfilled with hydrogen three times and then stirred at room temperature under 1 atm hydrogen for 30 minutes. The reaction mixture was filtered through a pad of Celite and the filtrate was concentrated to dryness. The resulting material was triturated with 9:1 heptane:EtOAc, filtered, and concentrated in vacuo to afford 89 mg of product. 4-[1-(4-fluorophenyl)-5-hydroxy-2-(2-methoxy-1,1-dimethyl-ethyl)indol-3-yl]benzoic acid (85%). ¹H NMR (400 MHz, DMSO-d6) δ 12.96 (s, 1H), 8.67 (s, 1H), 8.02 (d, J=8.2 Hz, 3H), 7.61-7.36 (m, 7H), 6.53 (dd, J=8.7, 2.3 Hz, 1H), 6.37 (d, J=8.7 Hz, 1H), 6.26 (d, J=2.3 Hz, 1H), 3.01 (s, 3H), 2.99 (s, 4H), 1.05 (s, 7H). ESI-MS m/z calc. 433.17, found 434.32 (M+1)⁺.

### Compound 165

### Synthesis of 4-(2-(I-cyano-2-methylpropan-2-yl)-1-(4-fluorophenyl)-5-hydroxy-1H-indol-3-yl)benzoic acid (165)

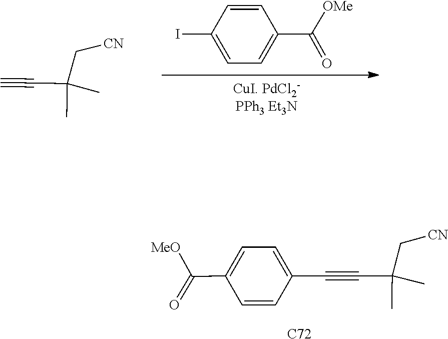

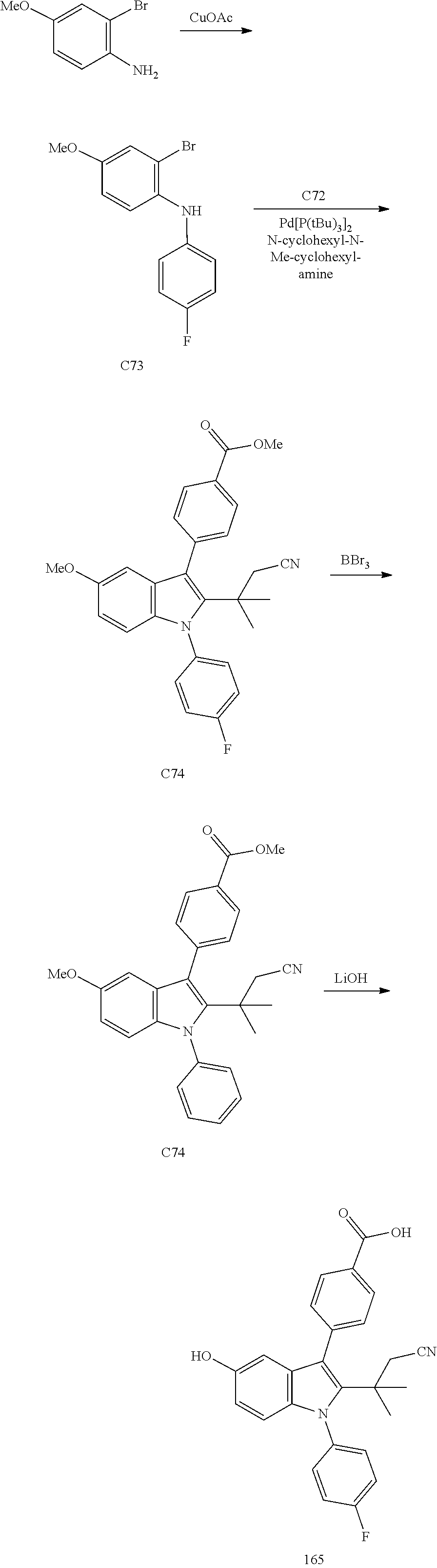

### Step 1. Synthesis of methyl 4-(4-cyano-3,3-dimethylbut-1-yn-1-yl)benzoate (C72)

A solution of benzyl 4-iodobenzoate (15.00 g, 44.36 mmol), Pd(PPh₃)₂Cl₂ (0.94 g, 1.33 mmol), and CuI (0.51 g, 2.66 mmol) in triethylamine (100 mL) and THE (100 mL) was purged with nitrogen for 5 minutes. To the mixture was added 4-methoxy-3,3-dimethyl-but-1-yne (7.22 g, 64.37 mmol). The reaction mixture was purged with nitrogen for 2 minutes. The flask was sealed and heated to 50° C. for 2 hours. The mixture was filtered and the solid was washed twice with EtOAc. The filtrate was concentrated in vacuo. The resulting residue was purified by silica gel chromatography (330 g ISCO column) using 0-50% EtOAc/heptanes gradient to afford 13 g of product. Benzyl 4-(4-methoxy-3,3-dimethyl-but-1-ynyl)benzoate (79%). ¹H NMR (300 MHz, Chloroform-d) δ 8.04-7.94 (m, 2H), 7.51-7.32 (m, 7H), 5.37 (s, 2H), 3.46 (q, 3H), 3.36 (q, 2H), 1.33 (s, 6H). ESI-MS m/z calc. 322.2, found 323.1 (M+1)⁺.

### Step 2. Synthesis of 2-bromo-N-(4-fluorophenyl)-4-methoxyaniline (C73)

At ambient temperature, a 50 mL round bottom flask was charged with 2-bromo-4-methoxy-aniline (0.52 g, 2.57 mmol), (4-fluorophenyl)boronic acid (0.73 g, 5.18 mmol), copper (II) acetate (0.94 g, 5.15 mmol) and 4 A Sieves (0.47 g). Dichloromethane (15 mL) was added to the mixture and the slurry was stirred open to the air for 15 minutes. Triethylamine (0.89 mL, 6.39 mmol) was added dropwise at ambient temperature and the resulting dark purple mixture was stirred open to the air overnight. The mixture was filtered through a pad of celite and washed with CH₂Cl₂. The filtrate was washed with water and brine. The organic phase was washed with brine, dried (MgSO₄), filtered, and concentrated in vacuo. The resulting residue was purified by silica gel chromatography (80 g ISCO column) using 0-20% EtOAc/heptanes gradient to afford 543 mg of product. 2-bromo-N-(4-fluorophenyl)-4-methoxy-aniline (67%). ¹H NMR (400 MHz, Chloroform-d) δ 7.15 (d, J=2.8 Hz, 1H), 7.13 (s, 1H), 7.03-6.96 (m, 4H), 6.82 (dd, J=8.9, 2.8 Hz, 1H), 5.65 (s, 1H), 3.80 (s, 3H). ESI-MS m/z calc. 295.01, found 296.12 (M+1)⁺.

### Step 3. Synthesis of methyl 4-(2-(I-cyano-2-methylpropan-2-yl)-1-(4-fluorophenyl)-5-methoxy-1H-indol-3-yl)benzoate (C74)

A vial containing methyl 4-(4-cyano-3,3-dimethyl-but-1-ynyl)benzoate C72 (0.31 g, 1.29 mmol), 2-bromo-N-(4-fluorophenyl)-4-methoxy-aniline C73 (0.25 g, 0.84 mmol), and Pd[P(tBu)₃]₂ (0.02 g, 0.05 mmol) was evacuated and purged with nitrogen (2×). A solution of 1,4-dioxane (5 mL) and N-cyclohexyl-N-methyl-cyclohexanamine (0.45 mL, 2.10 mmol) was added and the reaction was stirred at 90° C. for 17 h. LCMS shows incomplete conversion to product. The mixture was cooled to room temperature and purged with nitrogen. Another 0.05 equivalents of Pd[P(tBu)₃]₂ (0.02 g, 0.04 mmol) and mixture was heated to 90° C. for 21 hours. The reaction was diluted with water and extracted with ethyl acetate. The organic phase was dried (MgSO₄), filtered, and concentrated in vacuo. The resulting residue was purified by silica gel chromatography (40 g ISCO column) using 0-50% EtOAc/heptanes gradient to afford 155 mg of product. Methyl 4-[2-(2-cyano-1,1-dimethyl-ethyl)-1-(4-fluorophenyl)-5-methoxy-indol-3-yl]benzoate (38%). ¹H NMR (400 MHz, Chloroform-d) δ 8.18 (d, J=8.2 Hz, 2H), 7.60 (d, J=8.2 Hz, 2H), 7.54-7.47 (m, 1H), 6.80 (dd, J=8.9, 2.5 Hz, 1H), 6.59 (d, J=8.9 Hz, 1H), 6.48 (d, J=2.4 Hz, 1H), 4.00 (s, 3H), 3.72 (s, 3H), 2.44 (s, 2H), 1.31 (s, 6H). ESI-MS m/z calc. 456.18, found 457.36 (M+1)⁺.

### Step 4. methyl 4-(2-(I-cyano-2-methylpropan-2-yl)-1-(4-fluorophenyl)-5-hydroxy-1H-indol-3-yl)benzoate (C75)

To a cold (0° C.) solution of methyl 4-[2-(2-cyano-1,1-dimethyl-ethyl)-1-(4-fluorophenyl)-5-methoxy-indol-3-yl]benzoate C74 (0.093 g, 0.204 mmol) in dichloromethane (5.5 mL) was added tribromoborane (0.300 mL of 1 M solution, 0.300 mmol). The reaction mixture was stirred for 4 hours at room temperature. LCMS after shows product (minor) and starting material (major). Additional tribromoborane (0.200 mL of 1 M solution, 0.200 mmol) was added and the reaction was stirred at room temperature for another 1 hour. The reaction vial was cooled to 0° C. and quenched with aqueous saturated NaHCO₃ solution. The organic layer was washed with brine, dried over MgSO₄, filtered through a phase separator, and concentrated in vacuo. The resulting residue was purified by silica gel chromatography (40 g ISCO column) using 0-20% EtOAc/heptanes gradient to afford 42 mg of product. Methyl 4-[2-(2-cyano-1,1-dimethyl-ethyl)-1-(4-fluorophenyl)-5-hydroxy-indol-3-yl]benzoate (35%). ¹H NMR (400 MHz, Chloroform-d) δ 8.19-8.13 (m, 2H), 7.61-7.55 (m, 2H), 7.53-7.46 (m, 2H), 7.33-7.29 (m, 2H), 6.72 (dd, J=8.7, 2.5 Hz, 1H), 6.55 (dd, J=8.7, 0.6 Hz, 1H), 6.46 (dd, J=2.5, 0.6 Hz, 1H), 4.49 (s, 1H), 4.00 (s, 3H), 2.44 (s, 2H), 1.31 (s, 6H). ESI-MS m/z calc. 442.16928, found 443.23 (M+1)⁺.

### Step 5. Synthesis of 4-(2-(1-cyano-2-methylpropan-2-yl)-1-(4-fluorophenyl)-5-hydroxy-1H-indol-3-yl)benzoic acid (165)

To a solution of methyl 4-[2-(2-cyano-1,1-dimethyl-ethyl)-1-(4-fluorophenyl)-5-hydroxy-indol-3-yl]benzoate C75 (0.040 g, 0.069 mmol) in water (0.5 mL), THE (0.5 mL) and MeOH (1 mL) was added lithium hydroxide Ion (0.015 g, 0.626 mmol). The reaction mixture was stirred at room temperature for 5 hours. The mixture was concentrated, diluted in water, acidified using 6N HCl, and extracted with ethyl acetate. The organic phase was dried (MgSO₄), filtered, and concentrated in vacuo. The product was then triturated using 9:1 heptanes:ethyl acetate to afford 10 mg of product. 4-[2-(2-cyano-1,1-dimethyl-ethyl)-1-(4-fluorophenyl)-5-hydroxy-indol-3-yl]benzoic acid (32%). ¹H NMR (400 MHz, Chloroform-d) δ 8.25-8.17 (m, 2H), 7.63-7.56 (m, 2H), 7.52-7.44 (m, 2H), 7.31-7.26 (m, 2H), 6.71 (dd, J=8.7, 2.5 Hz, 1H), 6.53 (d, J=8.8 Hz, 1H), 6.45 (d, J=2.4 Hz, 1H), 2.43 (s, 2H), 1.30 (s, 6H). ESI-MS m/z calc. 428.15, found 429.28 (M+1)⁺.

### Compound 166

### Synthesis of 4-(1-(4-fluoro-3-methylphenyl)-5-hydroxy-2-(tetrahydro-2H-pyran-4-yl)-1H-indol-3-yl)benzoic acid (165)

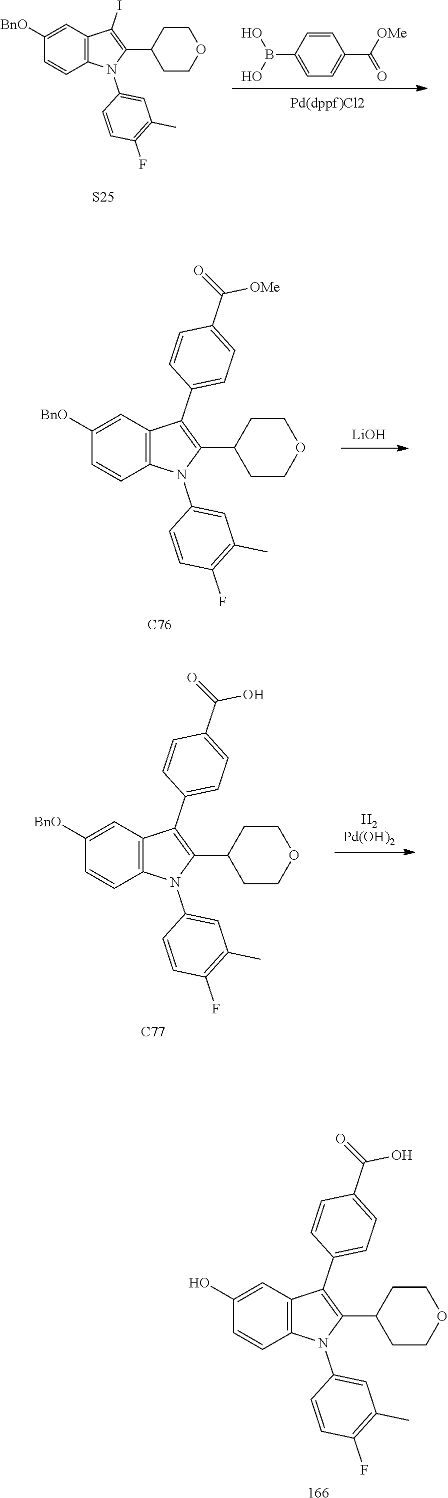

### Step 1. Synthesis of methyl 4-(5-(benzyloxy)-1-(4-fluoro-3-methylphenyl)-2-(tetrahydro-2H-pyran-4-yl)-1H-indol-3-yl)benzoate (C76)

To a solution of 5-benzyloxy-1-(4-fluoro-3-methyl-phenyl)-3-iodo-2-tetrahydropyran-4-yl-indole S25 (0.062 g, 0.109 mmol), (4-methoxycarbonylphenyl)boronic acid (0.022 g, 0.122 mmol) and sodium carbonate (0.110 mL of 2 M solution, 0.220 mmol) in DMF (1 mL) was added Pd(dppf)Cl₂—CH₂Cl₂ (0.009 mg, 0.011 mmol). The reaction mixture was heated to 100° C. and stirred at this temperature overnight. The mixture was diluted into EtOAc and water and filtered through celite. The aqueous phase was extracted with EtOAc. The combined organic phases were washed with water (2×), brine, dried over sodium sulfate, filtered and concentrated in vacuo. The resulting residue was purified by silica gel chromatography using 0-35% EtOAc/heptanes gradient to afford 45 mg of product. Methyl 4-[5-benzyloxy-1-(4-fluoro-3-methyl-phenyl)-2-tetrahydropyran-4-yl-indol-3-yl]benzoate (71%). ¹H NMR (400 MHz, Chloroform-d) δ 8.15 (d, J=8.2 Hz, 2H), 7.52 (d, J=8.2 Hz, 2H), 7.42 (d, J=7.2 Hz, 2H), 7.36 (t, J=7.5 Hz, 2H), 7.31 (d, J=7.0 Hz, 1H), 7.20 (dd, J=13.7, 7.0 Hz, 3H), 6.92 (d, J=2.4 Hz, 1H), 6.87-6.83 (m, 1H), 6.77 (d, J=8.8 Hz, 1H), 5.00 (s, 2H), 3.98 (s, 3H), 3.83 (d, J=11.5 Hz, 2H), 3.20 (t, J=11.4 Hz, 2H), 3.00 (d, J=12.4 Hz, 1H), 2.39-2.34 (m, 3H), 1.79 (d, J=13.0 Hz, 2H), 1.60 (d, J=12.3 Hz, 2H). ESI-MS m/z calc. 549.23, found 550.49 (M+1)⁺.

### Step 2. Synthesis of methyl 4-(5-(benzyloxy)-1-(4-fluoro-3-methylphenyl)-2-(tetrahydro-2H-pyran-4-yl)-1H-indol-3-yl)benzoate (C77)

To a solution of methyl 4-[5-benzyloxy-1-(4-fluoro-3-methyl-phenyl)-2-tetrahydropyran-4-yl-indol-3-yl]benzoate C76 (0.045 g, 0.078 mmol) in THE (1.6 mL)/methanol (1.6 mL) was added LiOH (0.800 mL of 1 M solution, 0.800 mmol). The reaction mixture was heated 50° C. and stirred at this temperature overnight. The mixture was concentrated under reduced pressure. 1 mL of water was added and the mixture acidified to pH 5 with 1 N HCl. The mixture was extracted three times with CH₂Cl₂. The combined organic phases were dried (MgSO₄), filtered, and concentrated in vacuo to afford 40 mg of product. 4-[5-benzyloxy-1-(4-fluoro-3-methyl-phenyl)-2-tetrahydropyran-4-yl-indol-3-yl]benzoic acid (95%). ¹H NMR (400 MHz, Chloroform-d) δ 8.20-8.14 (m, 2H), 7.52-7.46 (m, 2H), 7.37-7.33 (m, 2H), 7.32-7.25 (m, 2H), 7.26-7.19 (m, 1H), 7.17-7.10 (m, 3H), 6.87 (d, J=2.3 Hz, 1H), 6.80 (dd, J=8.8, 2.4 Hz, 1H), 6.71 (d, J=8.8 Hz, 1H), 4.94 (s, 2H), 3.86-3.77 (m, 2H), 3.72-3.66 (m, 1H), 3.22-3.11 (m, 2H), 2.99-2.87 (m, 1H), 2.33-2.26 (m, 3H), 1.83-1.70 (m, 3H), 1.60-1.48 (m, 2H). ESI-MS m/z calc. 535.22, found 536.49 (M+1)⁺.

### Step 3. Synthesis of 4-(1-(4-fluoro-3-methylphenyl)-5-hydroxy-2-(tetrahydro-2H-pyran-4-yl)-1H-indol-3-yl)benzoic acid (166)

To a solution of 4-[5-benzyloxy-1-(4-fluoro-3-methyl-phenyl)-2-tetrahydropyran-4-yl-indol-3-yl]benzoic acid C77 (0.040 g, 0.074 mmol) in MeOH (1 mL) was added dihydroxypalladium (0.002 g, 0.014 mmol). The mixture was placed under 1 atmosphere of hydrogen atmosphere and stirred for 1 hour. The mixture was filtered through a pad of celite and then a pad of florisil. The filtrate was concentrated in vacuo. The resulting residue was purified by silica gel chromatography (4 g ISCO column) using 0-10% MeOH/CH₂Cl₂ gradient to afford 28 mg of product. 4-[1-(4-fluoro-3-methyl-phenyl)-5-hydroxy-2-tetrahydropyran-4-yl-indol-3-yl]benzoic acid (83%). ¹H NMR (400 MHz, Methanol-d4) δ 8.16-8.06 (m, 2H), 7.53-7.44 (m, 2H), 7.31-7.17 (m, 3H), 6.78 (dd, J=2.0, 1.0 Hz, 1H), 6.72-6.64 (m, 2H), 3.83 (d, J=11.2 Hz, 2H), 3.29-3.19 (m, 2H), 3.07-2.96 (m, 1H), 2.42-2.34 (m, 3H), 1.89-1.75 (m, 2H), 1.69-1.57 (m, 2H). ESI-MS m/z calc. 445.17, found 446.49 (M+1)⁺.

### Compounds 167-176

Compounds 167-176 (Table 13) were prepared by Suzuki coupling of the appropriate boronic acid with the relevant iodoindole intermediate as described for the preparation of compound 166.

TABLE 13
Structure and physicochemical data for compounds 167-176

| | | Boronic | |
|---|---|---|---|
| Compound | Method/Product | Acid | ¹H NMR; LCMS m/z [M + H]⁺ |
| 167 | 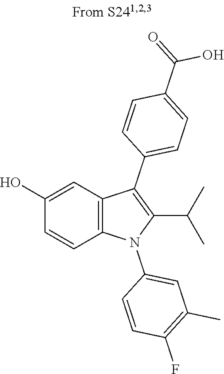 [SMILES: Cc1cc(-n2c(C(C)C)c(-c3ccc(C(=O)O)cc3)c3cc(O)ccc32)ccc1F] | 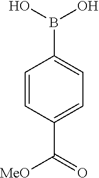 [SMILES: COC(=O)c1ccc(B(O)O)cc1] | ¹H NMR (400 MHz, Chloroform-d) δ 8.24-8.13 (m, 2H), 7.63-7.54 (m, 2H), 7.25- 7.12 (m, 3H), 6.85 (dd, J = 2.4, 0.6 Hz, 1H), 6.81-6.65 (m, 2H), 3.17 (s, 1H), 2.37 (d, J = 2.0 Hz, 3H), 1.13 (dd, J = 7.2, 2.9 Hz, 6H). LCMS m/z 403.1 [M + H]⁺. |
| 168 | 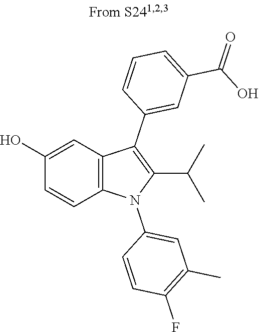 [SMILES: Cc1cc(-n2c(C(C)C)c(-c3cccc(C(=O)O)c3)c3cc(O)ccc32)ccc1F] | 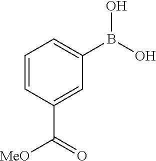 [SMILES: COC(=O)c1cccc(B(O)O)c1] | ¹H NMR (400 MHz, Chloroform-d) δ 8.23 (d, J = 2.0 Hz, 1H), 8.14-8.07 (m, 1H), 7.76-7.65 (m, 1H), 7.57 (t, J = 7.7 Hz, 1H), 7.26-7.12 (m, 3H), 6.83-6.63 (m, 3H), 3.17- 3.05 (m, 1H), 2.37 (d, J = 1.9 Hz, 3H), 1.12 (dd, J = 7.4, 3.1 Hz, 6H). LCMS m/z 403.1 [M + H]⁺. |
| 169 | 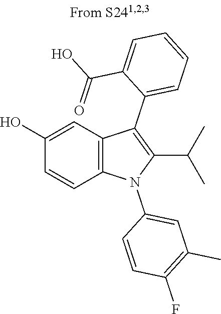 [SMILES: Cc1cc(-n2c(C(C)C)c(-c3ccccc3C(=O)O)c3cc(O)ccc32)ccc1F] | 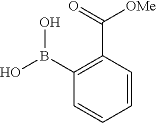 [SMILES: COC(=O)c1ccccc1B(O)O] | ¹H NMR (400 MHz, Chloroform-d) δ 8.24 (s, 1H), 7.65-7.57 (m, 1H), 7.57-7.48 (m, 1H), 7.46-7.37 (m, 1H), 7.24-7.10 (m, 3H), 6.82-6.72 (m, 1H), 6.72-6.65 (m, 1H), 6.52 (d, J = 2.3 Hz, 1H), 2.98 (s, 1H), 2.41-2.32 (m, 3H), 1.10- 0.95 (m, 6H). LCMS m/z 403.1 [M + H]⁺. |
| 170 | 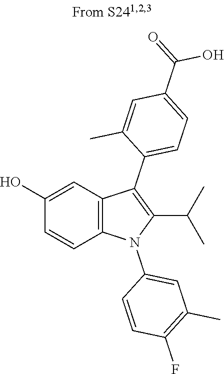 [SMILES: Cc1cc(-n2c(C(C)C)c(-c3ccc(C(=O)O)cc3C)c3cc(O)ccc32)ccc1F] | 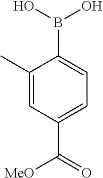 | ¹H NMR (400 MHz, Chloroform-d) δ 8.09 (s, 1H), 7.99 (d, J = 7.9 Hz, 1H), 7.44 (d, J = 7.9 Hz, 1H), 7.19 (dd, J = 11.2, 6.7 Hz, 3H), 6.80 (d, J = 8.7 Hz, 1H), 6.68 (dd, J = 8.8, 2.3 Hz, 1H), 6.47 (t, J = 1.7 Hz, 1H), 2.94 (s, 1H), 2.37 (s, 3H), 2.25 (s, 3H), 1.18-1.08 (m, 3H), 0.96 (d, J = 7.1 Hz, 3H). LCMS m/z 416.8 [M + H]⁺. |
| 171 | 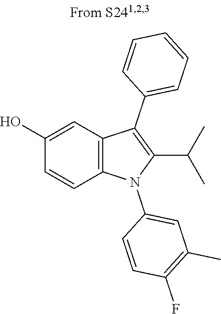 | 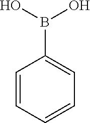 | ¹H NMR (400 MHz, Chloroform-d) δ 8.09 (s, 1H), 7.99 (d, J = 7.9 Hz, 1H), 7.44 (d, J = 7.9 Hz, 1H), 7.19 (dd, J = 11.2, 6.7 Hz, 3H), 6.80 (d, J = 8.7 Hz, 1H), 6.68 (dd, J = 8.8, 2.3 Hz, 1H), 6.47 (t, J = 1.7 Hz, 1H), 2.94 (s, 1H), 2.37 (s, 3H), 2.25 (s, 3H), 1.18-1.08 (m, 3H), 0.96 (d, J = 7.1 Hz, 3H). LCMS m/z 416.8 [M + H]⁺ |
| 172 | 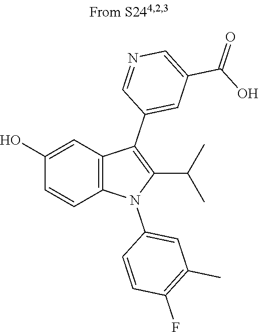 [SMILES: Cc1cc(-n2c(C(C)C)c(-c3cncc(C(=O)O)c3)c3cc(O)ccc32)ccc1F] | 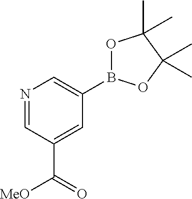 | ¹H NMR (400 MHz, Methanol- d4) δ 9.24 (s, 1H), 9.03 (s, 1H), 8.96 (t, J = 1.9 Hz, 1H), 7.34 (t, J = 7.0 Hz, 1H), 7.31-7.26 (m, 2H), 6.80 (dd, J = 1.9, 1.0 Hz, 1H), 6.72-6.68 (m, 2H), 3.19 (q, J = 7.2 Hz, 1H), 2.40-2.35 (m, 3H), 1.19 (dd, J = 7.2, 1.3 Hz, 6H). LCMS m/z 405.3 [M + H]⁺ |
| 173 | 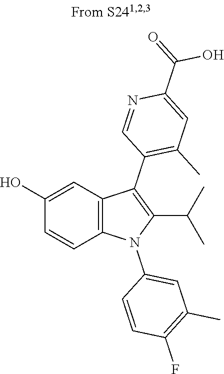 [SMILES: Cc1cc(-n2c(C(C)C)c(-c3cnc(C(=O)O)cc3C)c3cc(O)ccc32)ccc1F] | 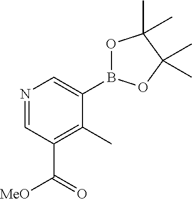 | ¹H NMR (400 MHz, Methanol- d4) δ 8.65 (s, 1H), 8.44 (s, 1H), 7.34 (d, J = 6.7 Hz, 1H), 7.30- 7.25 (m, 2H), 6.74 (d, J = 8.8 Hz, 1H), 6.67 (dd, J = 8.7, 2.2 Hz, 1H), 6.36 (d, J = 2.3 Hz, 1H), 3.04-2.93 (m, 1H), 2.47 (d, J = 2.4 Hz, 3H), 2.38 (t, J = 2.5 Hz, 3H), 1.19-1.11 (m, 3H), 0.99 (d, J = 7.1 Hz, 3H). LCMS m/z 418.6 [M + H]⁺ |
| 174 | 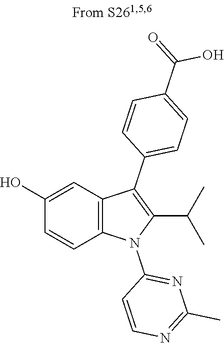 [SMILES: Cc1nccc(-n2c(C(C)C)c(-c3ccc(C(=O)O)cc3)c3cc(O)ccc32)n1] | 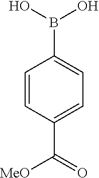 [SMILES: COC(=O)c1ccc(B(O)O)cc1] | ¹H NMR (400 MHz, Methanol- d4) δ 8.94 (d, J = 6.8 Hz, 1H), 8.24-8.16 (m, 2H), 7.99 (d, J = 6.8 Hz, 1H), 7.80-7.71 (m, 1H), 7.62-7.53 (m, 2H), 6.84 (dd, J = 8.9, 2.5 Hz, 1H), 6.55 (d, J = 2.4 Hz, 1H), 3.65-3.47 (m, 1H), 2.95 (s, 3H), 2.67 (s, 1H), 1.31 (d, J = 7.0 Hz, 6H). LCMS m/z 388.3 [M + H]⁺ |
| 175 | 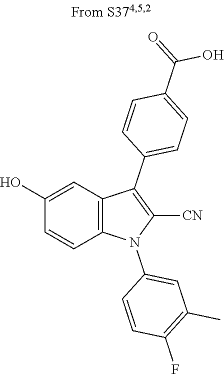 [SMILES: Cc1cc(-n2c(C#N)c(-c3ccc(C(=O)O)cc3)c3cc(O)ccc32)ccc1F] | 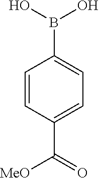 [SMILES: COC(=O)c1ccc(B(O)O)cc1] | ¹H NMR (400 MHz, DMSO-d6) δ 13.12 (s, 1H), 9.50 (s, 1H), 8.19-8.10 (m, 2H), 7.88-7.80 (m, 2H), 7.66 (dd, J = 7.3, 2.8 Hz, 1H), 7.60-7.50 (m, 1H), 7.46 (t, J = 9.0 Hz, 1H), 7.22 (d, J = 9.0 Hz, 1H), 7.16 (d, J = 2.2 Hz, 1H), 7.03 (dd, J = 9.0, 2.3 Hz, 1H), 2.36 (s, 3H). LCMS m/z 387.5 [M + H]⁺ |
| 176 | 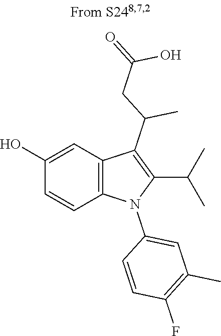 [SMILES: Cc1cc(-n2c(C(C)C)c(C(C)CC(=O)O)c3cc(O)ccc32)ccc1F] | 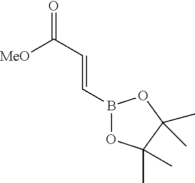 | ¹H NMR (400 MHz, Chloroform-d) δ 7.19-7.04 (m, 4H), 6.74 (d, J = 8.7 Hz, 1H), 6.64 (dd, J = 8.6, 2.2 Hz, 1H), 3.80 (q, J = 7.3 Hz, 1H), 3.08- 2.94 (m, 2H), 2.87 (dd, J = 15.3, 5.4 Hz, 1H), 2.35 (s, 3H), 1.55 (d, J = 6.9 Hz, 3H), 1.30 (t, J = 7.7 Hz, 6H). LCMS m/z 370.3 [M + H]⁺ |

¹Suzuki coupling: Pd(dppf)Cl₂—CH₂Cl₂, Na₂CO₃, H₂O, DMF at 100° C.
²Hydrolysis conditions: LiOH, THF, MeOH, H₂O
³Hydrogenation: H₂, Pd(OH)₂, MeOH
⁴Suzuki coupling: Pd(Ph₃P)₄, K₂CO₃, dioxane at 110° C.
⁵BBr₃, CH₂Cl₂, 0° C.
⁶Hydrolysis conditions: NaOH, MeOH
⁷Hydrogenation: H₂, Pd/C on wood or H₂, Pd/C, EtOAc
⁸Suzuki coupling: Pd(Ph₃P)₂Cl₂, Na₂CO₃, H₂O, DME at 80° C.

### Compound 177

### Synthesis of 4-(6-chloro-1-(4-fluorophenyl)-5-hydroxy-2-(1-methoxy-2-methylpropan-2-yl)-1H-indol-3-yl)benzoic acid (177)

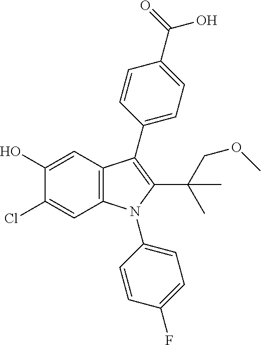 [SMILES: COCC(C)(C)c1c(-c2ccc(C(=O)O)cc2)c2cc(O)c(Cl)cc2n1-c1ccc(F)cc1]

To a solution of 4-[1-(4-fluorophenyl)-5-hydroxy-2-(2-methoxy-1,1-dimethyl-ethyl)indol-3-yl]benzoic acid 164 (0.025 g, 0.058 mmol) in MeCN (1.25 mL) was added 1-chloropyrrolidine-2,5-dione (0.015 g, 0.112 mmol). The reaction mixture was stirred at room temperature for 20 minutes and then at 45° C. for 1 hour. The crude mixture was purified by directly loading on to reverse phase HPLC to afford 3.3 mg of product. 4-[6-chloro-1-(4-fluorophenyl)-5-hydroxy-2-(2-methoxy-1,1-dimethyl-ethyl)indol-3-yl]benzoic acid (12%). ¹H NMR (400 MHz, Chloroform-d) δ 8.23-8.17 (m, 2H), 7.62-7.56 (m, 2H), 7.49-7.42 (m, 2H), 7.29 (s, 6H), 6.67 (s, 1H), 6.63 (s, 1H), 3.09 (s, 3H), 3.07 (s, 2H), 1.14 (s, 6H).

### Compound 178

### Synthesis of 4-(4-chloro-1-(4-fluorophenyl)-5-hydroxy-2-(I-methoxy-2-methylpropan-2-yl)-1H-indol-3-yl)benzoic acid (178)

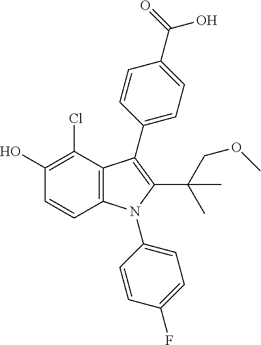 [SMILES: COCC(C)(C)c1c(-c2ccc(C(=O)O)cc2)c2c(Cl)c(O)ccc2n1-c1ccc(F)cc1]

To a solution of 4-[1-(4-fluorophenyl)-5-hydroxy-2-(2-methoxy-1,1-dimethyl-ethyl)indol-3-yl]benzoic acid 164 (0.029 g, 0.067 mmol) in NaOH (1.0 mL of 1 M solution, 1.0 mmol) was added sodium hypochlorite (0.130 mL of 5% w/v solution, 0.087 mmol). After 1 minute, the reaction mixture was diluted with water (1 mL) and HCl (1.5 mL of 1 M solution, 1.5 mmol). The mixture was extracted three times with EtOAc. The combined organic phases were dried (MgSO₄), filtered, and concentrated in vacuo. The crude material was triturated with 9:1 heptanes:EtOAc and filtered to afford 9.8 mg of product as an off-white solid. 4-[4-chloro-1-(4-fluorophenyl)-5-hydroxy-2-(2-methoxy-1,1-dimethyl-ethyl)indol-3-yl]benzoic acid (29%). ¹H NMR (400 MHz, Chloroform-d) δ 8.17-8.09 (m, 2H), 7.63 (d, J=8.2 Hz, 2H), 7.49-7.41 (m, 2H), 7.29-7.22 (m, 2H), 6.81 (d, J=8.8 Hz, 1H), 6.47 (d, J=8.8 Hz, 1H), 3.11 (s, 3H), 2.99 (s, 2H), 1.08 (s, 7H). ESI-MS m/z calc. 467.13, found 468.29 (M+1)⁺.

### Compound 179

### Synthesis of 4-(1-(4-fluorophenyl)-5-hydroxy-2-(1-methoxy-2-methylpropan-2-yl)-1H-indol-3-yl)benzoic acid (179)

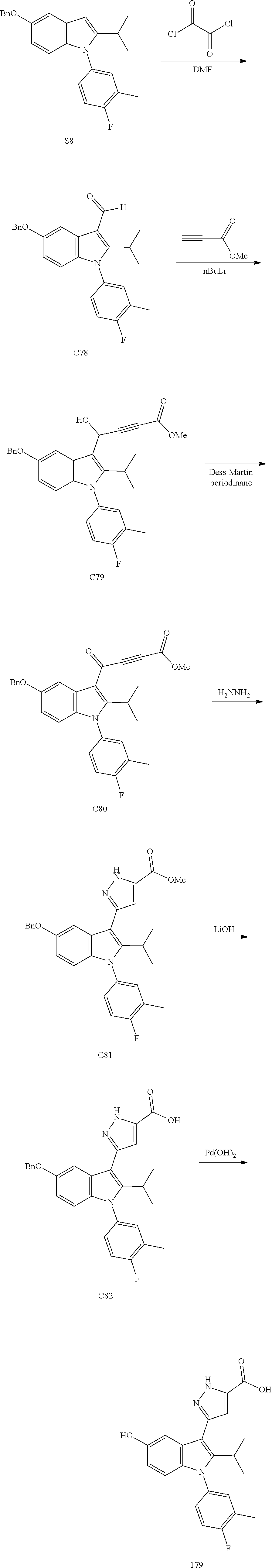

### Step 1. Synthesis of 4-methoxy-3,3-dimethylbut-1-yne (C78)

To a cold (0° C.) solution of oxalyl chloride (13.00 mL of 2 M, 26.00 mmol) in CH₂Cl₂ was added DMF (13 mL, 167.9 mmol). The suspension was stirred at 0° C. for 10 minutes. 5-benzyloxy-1-(4-fluoro-3-methyl-phenyl)-2-isopropyl-indole S8 (5.00 g, 13.39 mmol) in CH₂Cl₂ (50 mL) was added dropwise and the mixture was stirred at room temperature overnight. The solution was basified with aqueous saturated NaHCO₃ solution and extracted with three times with CH₂Cl₂. The organic phase was dried over Na₂SO₄, filtered and concentrated in vacuo. The resulting residue was purified by silica gel chromatography (80 g ISCO column) using 0-60% EtOAc/heptanes gradient to afford 4.67 g of product. 5-benzyloxy-1-(4-fluoro-3-methyl-phenyl)-2-isopropyl-indole-3-carbaldehyde (81%). ¹H NMR (300 MHz, Chloroform-d) δ 10.42 (s, 1H), 7.95 (d, J=2.5 Hz, 1H), 7.47-7.37 (m, 2H), 7.40-7.21 (m, 3H), 7.18-6.99 (m, 3H), 6.83 (dd, J=8.9, 2.5 Hz, 1H), 6.69 (dd, J=8.9, 0.5 Hz, 1H), 5.09 (s, 2H), 3.09 (p, J=7.2 Hz, 1H), 2.30 (d, J=2.0 Hz, 3H), 1.38 (dd, J=7.2, 2.1 Hz, 6H). ESI-MS m/z calc. 401.18, found 402.27 (M+1)⁺.

### Step 2. Synthesis of benzyl 4-(4-methoxy-3,3-dimethylbut-1-yn-1-yl)benzoate (C79)

To a cold (−78° C.) solution of methyl prop-2-ynoate (0.105 mL, 1.180 mmol) in THE (1 mL) was added n-butyllithium (0.470 mL of 2.5 M, 1.175 mmol). The reaction mixture was stirred at 30 minutes and a solution of 5-benzyloxy-1-(4-fluoro-3-methyl-phenyl)-2-isopropyl-indole-3-carbaldehyde C78 (0.335 g, 0.782 mmol) in THE (4 mL) was added dropwise. The mixture was stirred for 1 hour and −78° C. bath was switched to 0° C. and the mixture was stirred for 1 hour. The reaction mixture was quenched by addition of aqueous saturated NH₄Cl solution and extracted with EtOAc. The organic phase was dried (MgSO₄), filtered, and concentrated in vacuo. The resulting residue was purified by silica gel chromatography using 0-30% EtOAc/heptanes gradient to afford 154 mg of product. Methyl 4-[5-benzyloxy-1-(4-fluoro-3-methyl-phenyl)-2-isopropyl-indol-3-yl]-4-hydroxy-but-2-ynoate (40%). ¹H NMR (400 MHz, Chloroform-d) δ 7.56 (d, J=2.3 Hz, 1H), 7.52-7.48 (m, 2H), 7.41-7.36 (m, 2H), 7.34-7.30 (m, 1H), 7.18-7.05 (m, 3H), 6.85 (dd, J=8.9, 2.4 Hz, 1H), 6.77 (dd, J=8.8, 0.5 Hz, 1H), 6.08 (d, J=4.1 Hz, 1H), 5.15 (s, 2H), 3.77 (d, J=1.4 Hz, 3H), 3.07-3.00 (m, 1H), 2.34 (d, J=2.0 Hz, 3H), 2.21 (d, J=4.5 Hz, 1H), 1.36-1.28 (m, 6H). ESI-MS m/z calc. 485.20, found 486.01 (M+1)⁺.

### Step 3. Synthesis of 4-(benzyloxy)-2-bromo-N-(4-fluorophenyl)aniline (C80)

To a solution of methyl 4-[5-benzyloxy-1-(4-fluoro-3-methyl-phenyl)-2-isopropyl-indol-3-yl]-4-hydroxy-but-2-ynoate C79 (0.154 g, 0.310 mmol) in CH₂Cl₂ (2 mL) was added Dess-Martin periodinane (0.160 g, 0.377 mmol). The reaction mixture was stirred for 2 hours and 2-methyl-2-propanol (0.100 mL, 1.046 mmol) was added to expedite the reaction which was then stirred at room temperature overnight. The resulting residue was purified by silica gel chromatography (4 g ISCO column) using 0-20% EtOAc/heptanes gradient to afford 10 mg of product. Methyl 4-[5-benzyloxy-1-(4-fluoro-3-methyl-phenyl)-2-isopropyl-indol-3-yl]-4-oxo-but-2-ynoate (6%). ¹H NMR (400 MHz, Chloroform-d) δ 8.09 (d, J=2.4 Hz, 1H), 7.49 (d, J=7.7 Hz, 2H), 7.39 (t, J=7.4 Hz, 2H), 7.33 (d, J=7.2 Hz, 1H), 7.20 (t, J=8.7 Hz, 1H), 7.14 (t, J=6.5 Hz, 2H), 6.90 (dd, J=8.7, 2.5 Hz, 1H), 6.72 (d, J=8.9 Hz, 1H), 5.18 (s, 2H), 3.88 (s, 3H), 3.68-3.58 (m, 1H), 2.37 (s, 3H), 1.29 (d, J=2.3 Hz, 6H). ESI-MS m/z calc. 483.18, found 484.05 (M+1)⁺.

### Step 4. Synthesis of benzyl 4-(5-(benzyloxy)-1-(4-fluorophenyl)-2-(I-methoxy-2-methylpropan-2-yl)-1H-indol-3-yl)benzoate (C81)

To a solution of methyl 4-[5-benzyloxy-1-(4-fluoro-3-methyl-phenyl)-2-isopropyl-indol-3-yl]-4-oxo-but-2-ynoate C₉₀ (0.010 g, 0.019 mmol) in ethanol (0.5 mL) was added hydrazine hydrate (0.005 mL, 0.102 mmol). The reaction mixture was stirred at room temperature for 4 hours and the solvent was reduced under reduced pressure The resulting residue was purified by silica gel chromatography (4 g ISCO column) using 0-10% EtOAc/CH₂Cl₂ gradient to afford 8 mg of product. Methyl 3-[5-benzyloxy-1-(4-fluoro-3-methyl-phenyl)-2-isopropyl-indol-3-yl]-1H-pyrazole-5-carboxylate (78%). ¹H NMR (400 MHz, Chloroform-d) δ 10.34 (s, 1H), 7.47-7.42 (m, 2H), 7.41-7.35 (m, 2H), 7.34-7.30 (m, 1H), 7.21-7.12 (m, 3H), 6.98 (d, J=10.0 Hz, 2H), 6.89-6.76 (m, 2H), 5.04 (s, 2H), 4.00 (s, 3H), 3.15-3.06 (m, 1H), 2.36 (d, J=2.0 Hz, 3H), 1.16 (dd, J=7.1, 1.3 Hz, 6H). ESI-MS m/z calc. 497.21, found 498.03 (M+1)⁺.

### Step 5. Synthesis of benzyl 4-(5-(benzyloxy)-1-(4-fluorophenyl)-2-(1-methoxy-2-methylpropan-2-yl)-1H-indol-3-yl)benzoate (179)

To a solution of methyl 3-[5-benzyloxy-1-(4-fluoro-3-methyl-phenyl)-2-isopropyl-indol-3-yl]-1H-pyrazole-5-carboxylate C81 (0.008 g, 0.015 mmol) in THE (0.3 mL)/methanol (0.3 mL) was added lithium hydroxide (0.300 mL of 1 M, 0.300 mmol). The reaction mixture was heated to 50° C. and stirred overnight. The mixture was acidified with 1N HCl and extracted twice with EtOAc. The combined organic phases were washed with brine, dried over sodium sulfate, filtered and concentrated under in vacuo to afford 5 mg of product. 3-[5-benzyloxy-1-(4-fluoro-3-methyl-phenyl)-2-isopropyl-indol-3-yl]-1H-pyrazole-5-carboxylic acid (68%). ¹H NMR (400 MHz, Chloroform-d) δ 7.44 (d, J=7.4 Hz, 2H), 7.35 (t, J=7.4 Hz, 2H), 7.29 (d, J=7.2 Hz, 1H), 7.23-7.14 (m, 3H), 7.04-7.00 (m, 2H), 6.88 (dd, J=8.9, 2.3 Hz, 1H), 6.81 (d, J=8.8 Hz, 1H), 5.06 (s, 2H), 3.17-3.08 (m, 1H), 2.37 (d, J=1.9 Hz, 3H), 1.17 (d, J=7.1 Hz, 6H). ESI-MS m/z calc. 483.2, found 484.2 (M+1)⁺.

A mixture of 3-[5-benzyloxy-1-(4-fluoro-3-methyl-phenyl)-2-isopropyl-indol-3-yl]-1H-pyrazole-5-carboxylic acid (0.005 g, 0.010 mmol) and dihydroxypalladium (0.001 g, 0.007 mmol) in methanol (0.5 mL) was stirred under a hydrogen atmosphere for 1 hour. The crude mixture was filtered through a pad of celite and the filtrate was concentrated in vacuo to afford 3.9 mg of product. 3-[1-(4-fluoro-3-methyl-phenyl)-5-hydroxy-2-isopropyl-indol-3-yl]-1H-pyrazole-5-carboxylic acid (96%). ¹H NMR (400 MHz, Methanol-d4) δ 7.32-7.16 (m, 3H), 6.84 (s, 1H), 6.72 (d, J=2.2 Hz, 1H), 6.70-6.58 (m, 2H), 4.12 (d, J=12.2 Hz, 1H), 3.12-3.04 (m, 1H), 2.36 (d, J=1.9 Hz, 3H), 1.15 (d, J=7.1 Hz, 6H). ESI-MS m/z calc. 393.15, found 394.07 (M+1)⁺.

### Preparation 180

### Synthesis of 3-(1-(3,3-difluorocyclobutyl)-5-hydroxy-2-isopropyl-1H-indol-3-yl)benzoic acid (180)

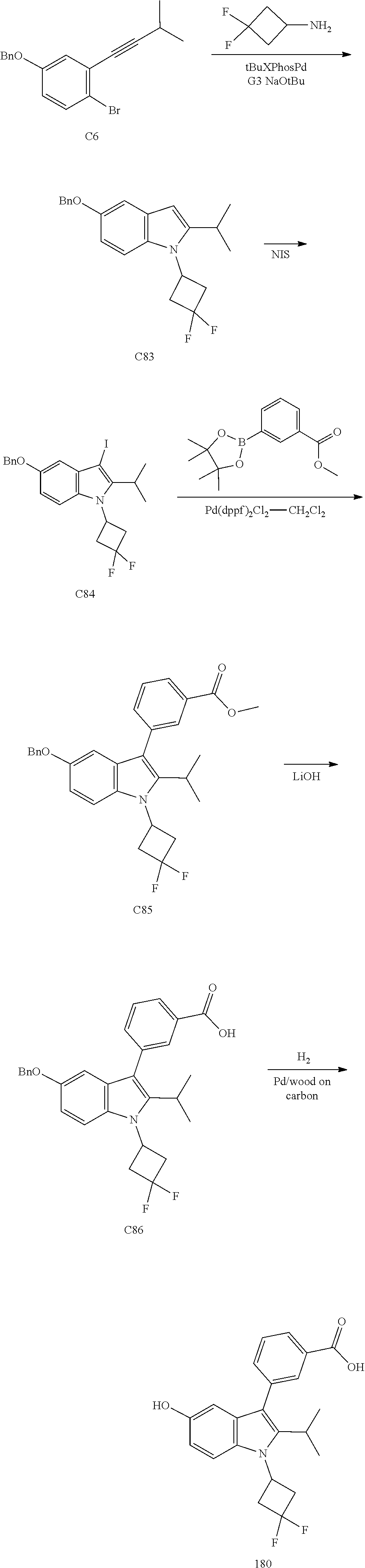

### Step 1. Synthesis of 5-(benzyloxy)-1-(3,3-difluorocyclobutyl)-2-isopropyl-1H-indole (C83)

To a solution 4-benzyloxy-1-bromo-2-(3-methylbut-1-ynyl)benzene C6 (0.23 g, 0.66 mmol) in tBuOH (2.5 mL)/Dioxane (1.3 mL) was added NaOtBu (0.26 g, 2.71 mmol). The mixture was purged with nitrogen and 3,3-difluorocyclobutanamine (Hydrochloride salt) (0.115 g, 0.801 mmol) and [2-(2-aminophenyl)phenyl]-methylsulfonyloxy-palladium; ditert-butyl-[2-(2,4,6-triisopropylphenyl)phenyl]phosphane (0.050 g, 0.063 mmol) were added. The mixture was heated at 80° C. for 18 hours. The reaction mixture was diluted into water (100 mL) and the aqueous layer was extracted three times with EtOAc (3×100 mL). The organic phase was dried (MgSO₄), filtered, and concentrated in vacuo. The resulting residue was purified by silica gel chromatography (24 g ISCO column) using 0-40% EtOAc/heptanes gradient to afford 195 mg of product. 4-benzyloxy-N-(3,3-difluorocyclobutyl)-2-(3-methylbut-1-ynyl)aniline (83%). ¹H NMR (400 MHz, Chloroform-d) δ 7.39 (tdd, J=16.9, 11.9, 8.8 Hz, 5H), 6.98 (d, J=2.9 Hz, 1H), 6.85 (dd, J=8.8, 2.9 Hz, 1H), 6.39 (d, J=8.9 Hz, 1H), 4.98 (s, 2H), 4.41 (d, J=5.6 Hz, 1H), 3.84 (q, J=7.9, 5.9 Hz, 1H), 3.06 (dtt, J=11.4, 7.9, 3.7 Hz, 2H), 2.86 (h, J=6.8 Hz, 1H), 2.60-2.40 (m, 2H), 1.31 (d, J=6.8 Hz, 6H). ESI-MS m/z calc. 355.17, found 356.71 (M+1)⁺. Indium tribromide (0.015 g, 0.042 mmol) was added to a solution of 4-benzyloxy-N-(3,3-difluorocyclobutyl)-2-(3-methylbut-1-ynyl)aniline (0.195 g) in toluene (2 mL) and the resulting solution was heated at 80° C. for 2 hours and then cooled to room temperature. The solvent was removed under reduced pressure. The resulting residue was purified by silica gel chromatography (24 g ISCO column) using 0-40% EtOAc/heptanes gradient to afford 140 mg of product. 5-benzyloxy-1-(3,3-difluorocyclobutyl)-2-isopropyl-indole (59%). ¹H NMR (400 MHz, Chloroform-d) δ 7.51-7.30 (m, 5H), 7.15 (d, J=2.5 Hz, 1H), 6.93 (dd, J=8.9, 2.6 Hz, 1H), 6.21 (t, J=0.8 Hz, 1H), 5.12 (s, 2H), 4.91 (tt, J=8.9, 4.4 Hz, 1H), 3.79-3.58 (m, 2H), 3.18-3.01 (m, 3H), 1.33 (d, J=6.8 Hz, 6H). ESI-MS m/z calc. 355.17, found 356.24 (M+1)⁺.

### Step 2. Synthesis of 5-(benzyloxy)-1-(3,3-difluorocyclobutyl)-3-iodo-2-isopropyl-1H-indole (C84)

A solution of N-iodosuccinimide (0.087 g, 0.387 mmol) and 5-benzyloxy-1-(3,3-difluorocyclobutyl)-2-isopropyl-indole C83 (0.140 g, 0.394 mmol) in CH₂Cl₂ (4.0 mL) was purged with a stream of nitrogen. The reaction vial was sealed and stirred for 30 minutes. The mixture was concentrated in vacuo. The resulting residue was purified by silica gel chromatography (12 g ISCO column) using 0-50% EtOAc/heptanes gradient to afford 166 mg of product. 5-benzyloxy-1-(3,3-difluorocyclobutyl)-3-iodo-2-isopropyl-indole (88%). ¹H NMR (400 MHz, Chloroform-d) δ 7.52 (ddt, J=7.5, 1.4, 0.7 Hz, 2H), 7.45-7.39 (m, 3H), 7.38-7.34 (m, 1H), 7.03 (d, J=2.5 Hz, 1H), 6.98 (dd, J=8.9, 2.6 Hz, 1H), 5.16 (s, 2H), 5.11 (dt, J=8.9, 4.8 Hz, 1H), 3.72-3.52 (m, 3H), 3.22-3.04 (m, 2H), 1.46 (d, J=7.3 Hz, 6H).

### Step 3. Synthesis of methyl 3-(5-(benzyloxy)-1-(3,3-difluorocyclobutyl)-2-isopropyl-1H-indol-3-yl)benzoate (C85)

A suspension of 5-benzyloxy-1-(3,3-difluorocyclobutyl)-3-iodo-2-isopropyl-indole C84 (0.160 g, 0.332 mmol), methyl 3-(4,4,5,5-tetramethyl-1,3,2-dioxaborolan-2-yl)benzoate (0.130 g, 0.504 mmol) and Pd(dppf)Cl₂—CH₂Cl₂ (0.014 g, 0.017 mmol) was purged with nitrogen. DMF (2.0 mL) and Sodium carbonate (0.500 mL of 2 M, 1.000 mmol) were added. The reaction mixture was heated at 80° C. for 20 minutes. The volatiles were removed under reduced pressure and the residue was diluted into water and ethyl acetate were added. The resulting residue was purified by silica gel chromatography using 0-50% EtOAc/heptanes gradient to afford 130 mg of product. Methyl 3-[5-benzyloxy-1-(3,3-difluorocyclobutyl)-2-isopropyl-indol-3-yl]benzoate (80%). ¹H NMR (400 MHz, Chloroform-d) δ 8.07-8.01 (m, 2H), 7.57-7.46 (m, 3H), 7.46-7.29 (m, 5H), 7.09-6.86 (m, 2H), 5.11 (pd, J=8.9, 3.4 Hz, 1H), 5.01 (s, 2H), 3.93 (s, 3H), 3.83-3.66 (m, 2H), 3.38 (p, J=7.3 Hz, 1H), 3.13 (dddd, J=16.0, 13.2, 9.2, 4.0 Hz, 2H), 1.34 (d, J=7.3 Hz, 6H).

### Step 4. Synthesis of 3-(5-(benzyloxy)-1-(3,3-difluorocyclobutyl)-2-isopropyl-1H-indol-3-yl)benzoic acid (C86)

To a solution of methyl 3-[5-benzyloxy-1-(3,3-difluorocyclobutyl)-2-isopropyl-indol-3-yl]benzoate C85 (0.13 g, 0.27 mmol) in MeOH (2.20 mL), THF (0.80 mL) and H₂O (0.50 mL) was added lithium hydroxide (0.190 g, 4.53 mmol). The reaction mixture was stirred at 25° C. for 12 hours. The solvent was evaporated under reduced pressure and the white solid was dissolved in water (8 mL) and slowly acidified with HCl (2.5 mL of 2 M, 5.00 mmol). The aqueous layer was extracted three times with EtOAc. The organic phase was dried (MgSO₄), filtered, and concentrated in vacuo to afford 100 mg product. 3-[5-benzyloxy-1-(3,3-difluorocyclobutyl)-2-isopropyl-indol-3-yl]benzoic acid (75%). ¹H NMR (400 MHz, Chloroform-d) δ 8.15-8.04 (m, 2H), 7.67-7.47 (m, 3H), 7.45-7.28 (m, 5H), 7.01-6.90 (m, 2H), 5.12 (qt, J=9.1, 4.5 Hz, 1H), 5.02 (s, 2H), 3.74 (tdd, J=16.2, 12.9, 8.5 Hz, 2H), 3.39 (hept, J=7.0 Hz, 1H), 3.25-3.02 (m, 2H), 2.13 (s, 3H), 1.35 (d, J=7.3 Hz, 6H). ESI-MS m/z calc. 475.2, found 476.2 (M+1)⁺.

### Step 5. Synthesis of 3-(1-(3,3-difluorocyclobutyl)-5-hydroxy-2-isopropyl-1H-indol-3-yl)benzoic acid (180)

A solution of 3-[5-benzyloxy-1-(3,3-difluorocyclobutyl)-2-isopropyl-indol-3-yl]benzoic acid C86 (0.10 g, 0.20 mmol) in EtOAc (3.0 mL) and MeOH (1.0 mL) was purged with nitrogen. Pd/wood carbon (0.06 g of 10% w/w, 0.03 mmol) was added and the mixture was evacuated and filled with hydrogen. The mixture was stirred under an atmosphere of hydrogen for 2 hours. The mixture was filtered through a pad of celite and the filtrate was concentrated in vacuo. The resulting residue was purified by silica gel chromatography (4 g ISCO column) using 0-10% MeOH/CH₂Cl₂ gradient to afford 38 mg of product. 3-[1-(3,3-difluorocyclobutyl)-5-hydroxy-2-isopropyl-indol-3-yl]benzoic acid (48%). 1H NMR (400 MHz, Chloroform-d) δ 7.99 (tt, J=3.7, 1.9 Hz, 2H), 7.59-7.49 (m, 2H), 7.44 (d, J=8.6 Hz, 1H), 6.85-6.61 (m, 2H), 5.20 (tt, J=9.0, 4.6 Hz, 1H), 3.68 (tdd, J=16.2, 13.3, 8.4 Hz, 2H), 3.49-3.33 (m, 1H), 3.25-3.08 (m, 2H), 1.34 (d, J=7.3 Hz, 6H). ESI-MS m/z calc. 385.15, found 386.22 (M+1)⁺.

### Preparation 181

### Synthesis of (E)-3-(6-fluoro-1-(4-fluoro-3-methylphenyl)-5-hydroxy-2-isopropyl-1H-indol-3-yl)acrylic acid (181)

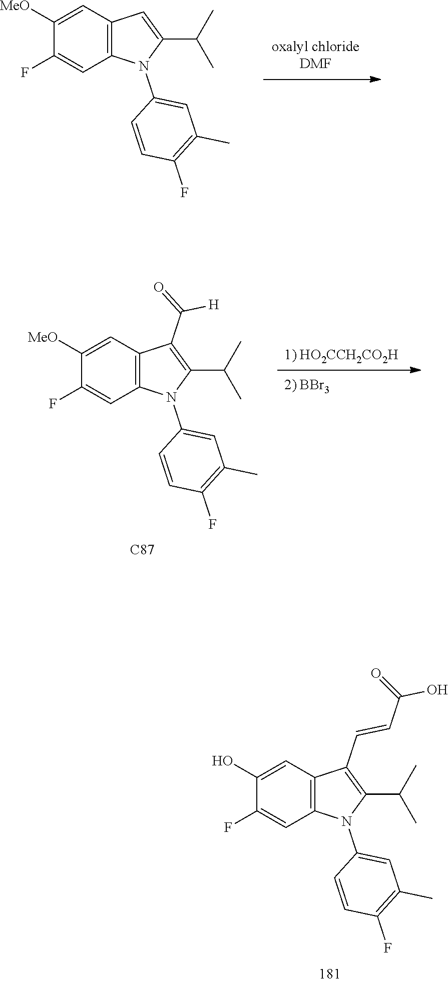 [SMILES: COc1cc2cc(C(C)C)n(-c3ccc(F)c(C)c3)c2cc1F ; COc1cc2c(C=O)c(C(C)C)n(-c3ccc(F)c(C)c3)c2cc1F ; "C87" ; Cc1cc(-n2c(C(C)C)c(/C=C/C(=O)O)c3cc(O)c(F)cc32)ccc1F]

### Step 1. Synthesis of 6-fluoro-1-(4-fluoro-3-methylphenyl)-2-isopropyl-5-methoxy-1H-indole-3-carbaldehyde (C87)

A solution of oxalyl chloride (13.0 mL of 2 M solution (1.8 mL of 2 M, 3.6 mmol) was added to a cold (0° C.) solution of DMF (1.6 mL, 20.66 mmol) in CH₂Cl₂ (5 mL). The solution was stirred at room temperature for 30 minutes. 6-fluoro-1-(4-fluoro-3-methyl-phenyl)-2-isopropyl-5-methoxy-indole (0.65 g, 2.05 mmol) in CH₂Cl₂ (10 mL) was added. The resulting solution was stirred at room temperature for 2 hours. Aqueous saturated NaHCO₃ solution (10 mL) was slowly added. The organic phase was dried (MgSO₄), filtered, and concentrated in vacuo. The resulting residue was purified by silica gel chromatography using 0-60% EtOAc/heptanes gradient to afford 0.58 g of product. 6-fluoro-1-(4-fluoro-3-methyl-phenyl)-2-isopropyl-5-methoxy-indole-3-carbaldehyde (80%). ¹H NMR (400 MHz, Chloroform-d) δ 10.50 (s, 1H), 8.01 (d, =8.4 Hz, 1H), 7.27 (d, J=9.8 Hz, 1H), 7.18-6.89 (m, 1H), 6.62 (d, J=11.0 Hz, 2H), 4.01 (s, 3H), 2.40 (d, J=2.0 Hz, 1H), 1.48 (d, J=2.6 Hz, 3H), 1.46 (d, J=2.6 Hz, 3H). ESI-MS m/z calc. 343.14, found 344.13 (M+1)⁺.

### Step 2. Synthesis of (E)-3-(6-fluoro-1-(4-fluoro-3-methylphenyl)-5-hydroxy-2-isopropyl-1H-indol-3-yl)acrylic acid (181)

To a solution of 6-fluoro-1-(4-fluoro-3-methyl-phenyl)-2-isopropyl-5-methoxy-indole-3-carbaldehyde C87 (0.05 g, 0.14 mmol) and malonic acid (0.10 g, 0.96 mmol) in pyridine (0.5 mL) was added piperidine (0.1 mL). The reaction mixture was heated in a closed vial for 24 h at 100° C. The mixture was poured into water (5 mL) and extracted twice with EtOAc. The organic phase was dried (MgSO₄), filtered, and concentrated in vacuo. The resulting residue was purified by silica gel chromatography using 0-60% EtOAc/heptanes gradient to afford 26 mg of product. (E)-3-[6-fluoro-1-(4-fluoro-3-methyl-phenyl)-2-isopropyl-5-methoxy-indol-3-yl]prop-2-enoic acid (46%). ESI-MS m/z calc. 371.13, found 372.16 (M+1)⁺.

To a solution of (E)-3-[6-fluoro-1-(4-fluoro-3-methyl-phenyl)-2-isopropyl-5-methoxy-indol-3-yl]prop-2-enoic acid (26 mg) in CH₂Cl₂ (3 mL) was added BBr₃ (0.427 mL of 1 M, 0.427 mmol). The reaction mixture was stirred at room temperature for 2 hours. The mixture was diluted with aqueous saturated NaHCO₃ solution (1 mL). The organic phase was dried (MgSO₄), filtered, and concentrated in vacuo. The crude residue was purified by reverse phase flash chromatography (RF ISCO, C₁₈ column, 30 g) eluting with CH₃CN/water (0-100%, 0.1% TFA) to afford 15 mg of product. (E)-3-[6-fluoro-1-(4-fluoro-3-methyl-phenyl)-5-hydroxy-2-isopropyl-indol-3-yl]prop-2-enoic acid (27%). ¹H NMR (400 MHz, Methanol-d4) δ 8.21 (d, J=15.8 Hz, 1H), 7.38 (d, J=8.3 Hz, 1H), 7.30-7.02 (m, 3H), 6.54 (d, J=11.0 Hz, 1H), 6.29 (d, J=15.8 Hz, 1H), 3.16-2.87 (m, 1H), 2.37 (d, J=2.1 Hz, 3H), 1.37 (d, J=2.2 Hz, 3H), 1.35 (d, J=2.2 Hz, 3H). ESI-MS m/z calc. 371.13, found 372.16 (M+1)⁺.

### Compound 182

### 1-(4-fluorophenyl)-5-hydroxy-2-methyl-1H-indole-3-carbonitrile (182)

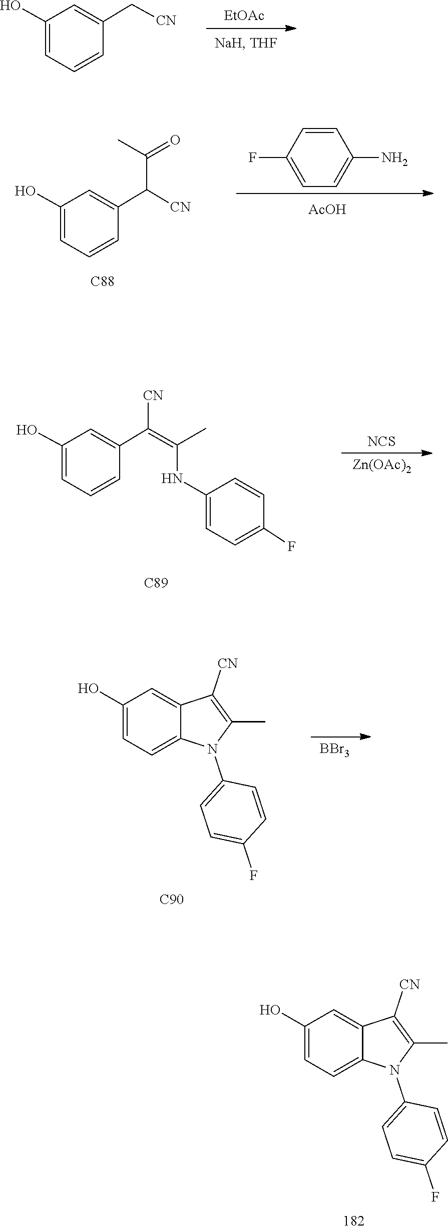

### Step 1. Synthesis of 2-(3-hydroxyphenyl)-3-oxobutanenitrile (C182)

To a cold (0° C.) solution of 2-(3-hydroxyphenyl)acetonitrile (3.00 g, 20.38 mmol) in THE (30 mL) was slowly added NaH (1.06 g of 60% w/w, 26.49 mmol). The cooling bath was then removed and the reaction mixture was stirred at room temperature for 1 hour. Ethyl acetate (2.39 mL, 24.46 mmol) was added in one lot. The reaction mixture was then heated to 60° C. for 3 hours. The mixture was cooled to room temperature and ⅔ of the solvent was removed under reduced pressure. The residue was dissolved in cold water (0° C., 50 mL). With vigorous stirring, 1N HCl solution was added dropwise until its pH reached neutral level. The aqueous phase was extracted three times with EtOAc. The combined organic phases were dried (MgSO₄), filtered and concentrated in vacuo. The resulting residue was purified by silica gel chromatography using 0-70% EtOAc/heptanes gradient to afford 3 g of product. 2-(3-hydroxyphenyl)-3-oxobutanenitrile (78%).

### Step 2. Synthesis of (E)-3-((4-fluorophenyl)amino)-2-(3-hydroxyphenyl)but-2-enenitrile (C89)

A mixture of 2-(3-hydroxyphenyl)-3-oxo-butanenitrile C88 (1.00 g, 5.28 mmol), 4-fluoroaniline (1.02 mL, 10.57 mmol) and acetic acid (0.60 mL, 10.57 mmol) in EtOH (10.00 mL) was stirred at 50° C. Additional 0.5 eq of 4-fluroaniline and 0.5 eq of acetic acid were added and the temperature was increased to 60° C. under reaction was complete. The mixture was evaporated to partially remove the solvent, the residue was poured into cold water (0° C., 40 mL) and neutralized with aqueous saturated NaHCO₃ solution. The combined organic phases were dried (MgSO₄), filtered and concentrated in vacuo. The resulting residue was purified by silica gel chromatography using 0-70% EtOAc/heptanes gradient to afford 900 mg of product (E)-3-(4-fluoroanilino)-2-(3-hydroxyphenyl)but-2-enenitrile (60%).

### Step 3. Synthesis of 1-(4-fluorophenyl)-5-hydroxy-2-methyl-1H-indole-3-carbonitrile (C90)

To a solution of (E)-3-(4-fluoroanilino)-2-(3-hydroxyphenyl)but-2-enenitrile C89 (0.70 g, 2.48 mmol) in 1,2-dichloroethane (7.0 mL) was added N-chlorosuccinimide (0.36 g, 2.72 mmol) in one portion. The reaction was stirred at room temperature until the total consumption of starting material. Then Zn(OAc)₂ hydrate (3.2 mmol) was added in one portion. The reaction temperature was gradually raised to reflux and stirred overnight. The reaction mixture was quenched with water and the aqueous was extracted with EtOAc. The combined organic phases were dried (MgSO₄), filtered and concentrated in vacuo. The resulting residue was purified by silica gel chromatography using 0-10% EtOAc/heptanes gradient to afford the desired product.

### Step 4. Synthesis of 1-(4-fluorophenyl)-5-hydroxy-2-methyl-1H-indole-3-carbonitrile (182)

To a cold (0° C.) solution of 1-(4-fluorophenyl)-5-hydroxy-2-methyl-indole-3-carbonitrile C90 (0.030 g, 0.107 mmol) in CH₂Cl₂ under nitrogen atmosphere (1.2 mL) was added tribromoborane (1.07 mL of 1 M, 1.07 mmol) dropwise. The reaction mixture was stirred for 90 minutes. Desired product observed. the reaction mixture was cooled to 0° C. and quenched with aqueous saturated NaHCO₃ solution slowly. The aqueous layer was extracted with CH₂Cl₂. The combined organic phases were dried (MgSO₄), filtered and concentrated in vacuo. The crude residue was purified by reverse phase flash chromatography (RF ISCO, C₁₈ column, 30 g) eluting with CH₃CN/water (0-100%, 0.1% TFA) to afford product. 1-(4-fluorophenyl)-5-hydroxy-2-methyl-indole-3-carbonitrile. ¹H NMR (400 MHz, DMSO-d6) δ 9.31 (s, 1H), 7.64-7.52 (m, 2H), 7.50-7.41 (m, 2H), 6.94-6.85 (m, 2H), 6.72 (dd, J=8.8, 2.3 Hz, 1H), 2.36 (s, 3H). ESI-MS m/z found 267.2 (M+1)⁺.

### Compound 183

### 2-(1-(4-fluorophenyl)-5-hydroxy-2-methyl-1H-indol-3-yl)propanenitrile (183)

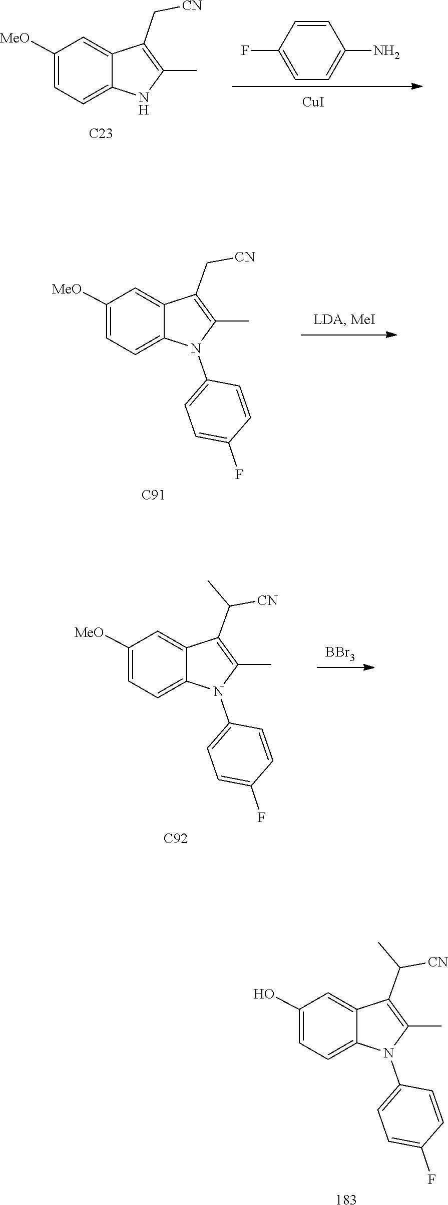

### Step 1. Synthesis of 2-(1-(4-fluorophenyl)-5-methoxy-2-methyl-1H-indol-3-yl)acetonitrile (C91)

To a suspension of 2-(5-methoxy-2-methyl-1H-indol-3-yl)acetonitrile C23 (1.32 g, 6.59 mmol) in toluene (13.20 mL) degassed for 10 minutes with nitrogen was added K₃PO₄ (4.20 g, 19.78 mmol), iodocopper (0.75 g, 3.96 mmol), N,N′-dimethylethane-1,2-diamine (0.42 mL, 3.96 mmol) and 1-fluoro-4-iodo-benzene (1.52 mL, 13.18 mmol). The pressure flask was sealed with a screw cap and the reaction mixture was heated at 110° C. for 16 hours. The reaction mixture was allowed to cool to room temperature and filtered through a plug of celite, with further washing with CH₂Cl₂. The filtrate was concentrated under reduced pressure. The resulting residue was purified by silica gel chromatography using 0-15% EtOAc/hexanes gradient to afford 845 mg of product. 2-[1-(4-fluorophenyl)-5-methoxy-2-methyl-indol-3-yl]acetonitrile (44%). ESI-MS m/z calc. 294.1, found 295.2 (M+1)⁺.

### Step 2. Synthesis of 2-(1-(4-fluorophenyl)-5-methoxy-2-methyl-1H-indol-3-yl)propanenitrile (C92)

To a cold (−78° C.) solution of 2-[1-(4-fluorophenyl)-5-methoxy-2-methyl-indol-3-yl]acetonitrile C91 (0.186 g, 0.632 mmol) in THE (2.8 mL) under a nitrogen atmosphere was added dropwise a solution of LDA (0.348 mL of 2 M in THF/heptane/ethylbenzene, 0.695 mmol). The reaction mixture was stirred for 30 minutes at −78° C. A solution of iodomethane (0.039 mL, 0.632 mmol) in THE (0.200 mL) was added dropwise. After 5 minutes, the reaction was quenched with aqueous saturated NH₄Cl solution and extracted twice with EtOAc. The organic phase was dried (MgSO₄), filtered, and concentrated in vacuo. The resulting residue was purified by silica gel chromatography using 0-15% EtOAc/hexanes gradient to afford 96 mg of product. 2-[1-(4-fluorophenyl)-5-methoxy-2-methyl-indol-3-yl]propanenitrile (49%). ESI-MS m/z calc. 308.1, found 309.3 (M+1)⁺.

### Step 3. Synthesis of 2-(1-(4-fluorophenyl)-5-hydroxy-2-methyl-1H-indol-3-yl)propanenitrile (183)

To a cold (−78° C.) solution of 2-[1-(4-fluorophenyl)-5-methoxy-2-methyl-indol-3-yl]propanenitrile C92 (0.095 g, 0.308 mmol) in CH₂Cl₂ (2 mL) under a nitrogen atmosphere was added dropwise boron tribromide (1.540 mL of 1 M, 1.540 mmol). The cooling bath was removed and the reaction continued stirring while warming to room temperature. After 1 hour, the reaction was cooled −78° C. and slowly quenched with MeOH. The mixture was partitioned between CH₂Cl₂ and aqueous saturated NaHCO₃ solution. The layers were separated and the aqueous layer was extracted again with CH₂Cl₂. The combined organic phases were dried over Na₂SO₄, filtered and concentrated in vacuo. The crude material was dissolved in DMF (1 mL) and purified by Waters mass directed LC/MS: (15-99% ACN/H₂O (5 mM HCl)). The desired fractions were partitioned between CH₂Cl₂/water, the layers were separated and the aqueous layer was extracted once more with CH₂Cl₂. The combined organic phases were dried over Na₂SO₄, filtered and concentrated in vacuo to afford 15 mg of product. 2-[1-(4-fluorophenyl)-5-hydroxy-2-methyl-indol-3-yl]propanenitrile (16%). ESI-MS m/z calc. 294.1, found 295.3 (M+1)⁺.

### Compound 184

### 1-(1-(4-fluorophenyl)-5-hydroxy-2-methyl-1H-indol-3-yl)cyclopropane-1-carbonitrile (184)

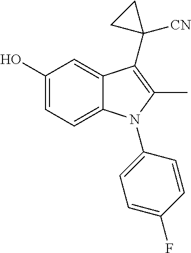 [SMILES: Cc1c(C2(C#N)CC2)c2cc(O)ccc2n1-c1ccc(F)cc1]

Compound 184 was prepared from 2-(1-(4-fluorophenyl)-5-methoxy-2-methyl-1H-indol-3-yl)propanenitrile C91 by alkylation with 1-bromo-2-chloroethane as described in the preparation of compound 183. LCMS m/z 307.4 [M+H]⁺.

### Compound 185

### 1-(4-fluorophenyl)-5-hydroxy-2-isopropyl-1H-indole-3-carbonitrile (185)

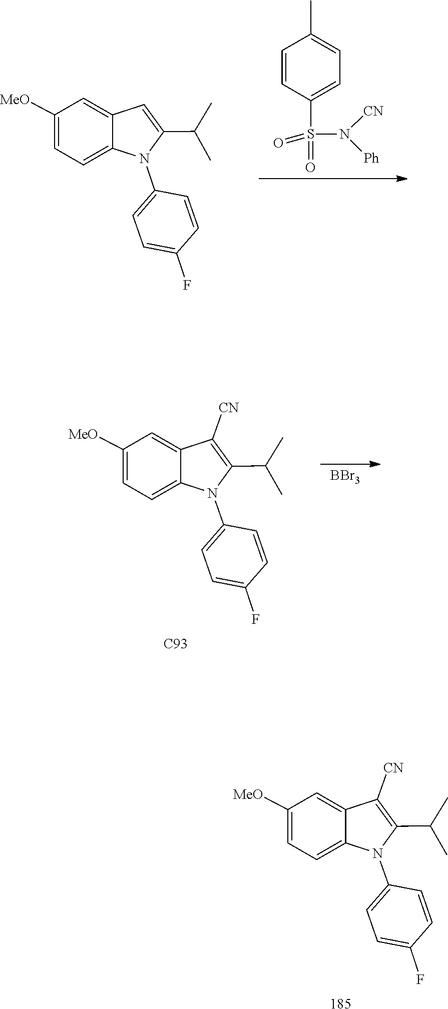

### Step 1. Synthesis of 2-(1-(4-fluorophenyl)-5-methoxy-2-methyl-1H-indol-3-yl)acetonitrile (C93)

1-(4-fluorophenyl)-2-isopropyl-5-methoxy-indole (0.126 g, 0.444 mmol) and N-cyano-4-methyl-N-phenyl-benzenesulfonamide (0.127 g, 0.444 mmol) were placed in a sealed vial with a Teflon pressure cap. The vial was evacuated and purged with nitrogen (3 cycles). Anhydrous DCE (0.446 mL) was added followed by the addition of boron trifluoride etherate (0.108 mL of 46.5% w/v, 0.355 mmol). The reaction mixture was heated at 80° C. for 16 hours. The reaction was cooled to room temperature the solvent was removed under reduced pressure. The resulting residue was purified by silica gel chromatography (40 g ISCO column) using 0-60% EtOAc/heptanes gradient to afford 32 mg of product. 1-(4-fluorophenyl)-2-isopropyl-5-methoxy-indole-3-carbonitrile (23%).

### Step 2. Synthesis 1-(4-fluorophenyl)-5-hydroxy-2-isopropyl-1H-indole-3-carbonitrile (185)

To a cold (−78° C.) solution of 1-(4-fluorophenyl)-2-isopropyl-5-methoxy-indole-3-carbonitrile C93 (0.032 g, 0.104 mmol) in CH₂Cl₂ (1.5 mL) under a nitrogen atmosphere was added dropwise boron tribromide (0.519 mL of 1 M solution in CH₂Cl₂, 0.519 mmol). The reaction mixture was stirred at −78° C. for 5 minutes and then gradually warmed to room temperature. After 1 hour at room temperature, the reaction cooled in an ice water bath and slowly quenched with aqueous saturated NaHCO₃ solution. The aqueous phase was extracted three times with CH₂Cl₂. The organic phase was dried (MgSO₄), filtered, and concentrated in vacuo. The crude material was dissolved in DMF (1 mL) and purified by Waters mass directed LC/MS: (15-99% ACN/H₂O (5 mM HCl)). The desired fractions were diluted with CH₂Cl₂. The aqueous phase was extracted once more with CH₂Cl₂. The combined organic phases were dried over Na₂SO₄, filtered and concentrated under reduced pressure to afford 22 mg of product. 1-(4-fluorophenyl)-5-hydroxy-2-isopropyl-indole-3-carbonitrile (71%). ¹H NMR (400 MHz, DMSO-d6) δ 9.31 (s, 1H), 7.62-7.52 (m, 2H), 7.52-7.43 (m, 2H), 6.91 (d, J=2.2 Hz, 1H), 6.77 (d, J=8.8 Hz, 1H), 6.71 (dd, J=8.8, 2.2 Hz, 1H), 2.87 (hept, J=7.1 Hz, 1H), 1.34 (d, J=7.0 Hz, 6H). ESI-MS m/z calc. 294.1, found 295.2 (M+1)⁺.

### Preparation of S45

### 2-(tert-butyl)-1-(4-fluorophenyl)-5-methoxy-1H-indole (S45)

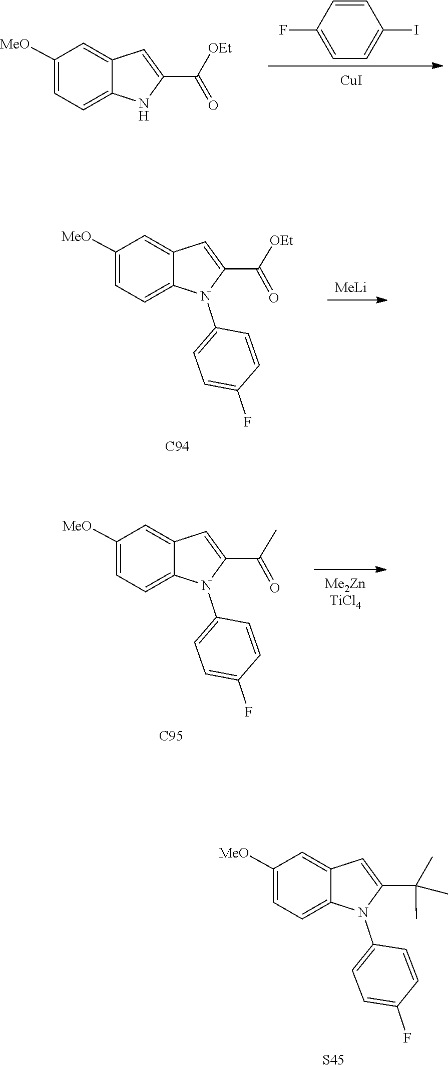

### Step 1. Synthesis of ethyl 1-(4-fluorophenyl)-5-methoxy-1H-indole-2-carboxylate (C94)

A solution of ethyl 5-methoxy-1H-indole-2-carboxylate (2.00 g, 9.12 mmol) in toluene (20.00 mL) was purged with nitrogen for 10 minutes. K₃PO₄ (5.81 g, 27.37 mmol), 1-fluoro-4-iodo-benzene (2.10 mL, 18.25 mmol), N,N′-dimethylethane-1,2-diamine (0.58 mL, 5.47 mmol) and copper (I) iodide (1.04 g, 5.47 mmol) were added sequentially to the degassing solution. The tube was then sealed and heated at 110° C. for 16 hours. The reaction mixture was cooled to room temperature and filtered through a pad of celite and further washed with CH₂Cl₂ (200 mL). The organic phase was dried (MgSO₄), filtered, and concentrated in vacuo. The resulting residue was purified by silica gel chromatography (80 g ISCO column) using 0-30% EtOAc/heptanes gradient to afford 2.49 g of product ethyl 1-(4-fluorophenyl)-5-methoxy-indole-2-carboxylate (87%).

### Step 2. Synthesis of I-(1-(4-fluorophenyl)-5-methoxy-1H-indol-2-yl)ethan-1-one (C95)

Under a nitrogen atmosphere, methyl lithium (9.78 mL of 1.6 M solution, 15.64 mmol) was slowly added to a cold (−30° C.) suspension of ethyl 1-(4-fluorophenyl)-5-methoxy-indole-2-carboxylate C94 (2.45 g, 7.82 mmol) in ether (24.50 mL). The reaction mixture was quenched with ammonium chloride. The layers were separated and the aqueous phase was extracted with ether. The combined organic layer was dried (MgSO₄), filtered and concentrated in vacuo. The resulting residue was purified by silica gel chromatography (40 g ISCO column) using 0-20% EtOAc/heptanes gradient to afford 0.5 g of product. 1-[1-(4-fluorophenyl)-5-methoxy-indol-2-yl]ethanone (23%). ESI-MS m/z calc. 283.1, found 284.4 (M+1)⁺.

### Step 3. Synthesis of 2-(tert-butyl)-1-(4-fluorophenyl)-5-methoxy-1H-indole (S45)

To a cold (−30 C) solution of TiCl₄ (6.35 mL of 1 M solution, 6.35 mmol) in CH₂Cl₂ (6.00 mL) was added dimethyl zinc (3.18 mL of 2 M solution, 6.35 mmol). After stirring for 10 minutes, 1-[1-(4-fluorophenyl)-5-methoxy-indol-2-yl]ethanone C95 (0.60 g, 2.12 mmol) was added. After 10 minutes, the reaction was warmed to 0° C. and then warmed to room temperature and stirred for an additional 3 hours. The reaction was poured onto ice and extracted with ether. The organic phase was washed with water and aqueous saturated NaHCO₃ solution. The organic phase was dried (MgSO₄), filtered, and concentrated in vacuo. The resulting residue was purified by silica gel chromatography using 0-20%0 EtOAc/heptanes gradient to afford 444 mg of product. 2-tert-butyl-1-(4-fluorophenyl)-5-methoxy-indole (710%). ESI-MS m/z calc. 297.2, found 298.0 (M+1)₁.

### Compounds 186-188

Compounds 186-188 were prepared as described for the preparation of compound 185. Any modifications are noted in the table footnotes.

TABLE 14
Structure and physicochemical data for compounds 186-188

| Compound | Product | Method | ¹H NMR; LCMS m/z [M + H]⁺ |
|---|---|---|---|
| 186 | 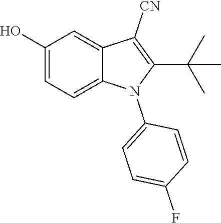 [SMILES: CC(C)(C)c1c(C#N)c2cc(O)ccc2n1-c1ccc(F)cc1] | From S45³ | ¹H NMR (400 MHz, DMSO-d6) δ 9.30 (s, 1H), 7.60-7.53 (m, 2H), 7.47- 7.41 (m, 2H), 6.90 (d, J = 2.2 Hz, 1H), 6.68 (dd, J = 8.8, 2.3 Hz, 1H), 6.45 (d, J = 8.8 Hz, 1H), 1.33 (s, 9H). LCMS m/z 309.4 [M + H]⁺. |
| 187 | 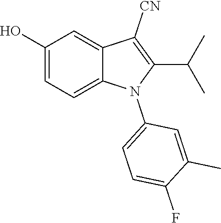 [SMILES: Cc1cc(-n2c(C(C)C)c(C#N)c3cc(O)ccc32)ccc1F] | FN^{1,3} | ¹H NMR (400 MHz, DMSO-d6) δ 9.31 (s, 1H), 7.49-7.44 (m, 1H), 7.41 (t, J = 8.9 Hz, 1H), 7.36 (ddd, J = 8.3, 4.7, 2.7 Hz, 1H), 6.91 (dd, J = 2.3, 0.6 Hz, 1H), 6.78 (dd, J = 8.8, 0.6 Hz, 1H), 6.71 (dd, J = 8.8, 2.3 Hz, 1H), 2.89 (p, J = 7.0 Hz, 1H), 2.33 (d, J = 2.0 Hz, 3H), 1.35 (dd, J = 7.0, 4.9 Hz, 6H). LCMS m/z 309.4 [M + H]⁺. |
| 188 | 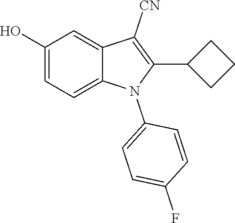 [SMILES: N#Cc1c(C2CCC2)n(-c2ccc(F)cc2)c2ccc(O)cc12] | From S35^{2,3} | ¹H NMR (300 MHz, DMSO-d6) δ 9.34 (s, 1H), 7.55-7.42 (m,, 3H), 6.92 (s, 1H), 6.72 (d, J = 9.0 Hz, 1H), 3.74- 3.55 (m, 1H), 3.35 (s, 1H), 2.43-2.36 (m,, 2H), 2.11-1.68 (m, 3H). LCMS m/z 307.2 [M + H]⁺. |

¹Method utilizes intermediate with different phenol protecting group (Me instead of Bn) prepared in same fashion as S8
²Photocoupling: Vaportech Easy Medchem flow reactor at a flow rate of 0.25 mL min irradiating with LED Gen 1 24 Watt @ 450 nm. (40 min residence time)
³BBr₃, CH₂Cl₂

### Compounds 189, 190 and 191

### 1-(4-fluorophenyl)-5-hydroxy-2-(1-hydroxypropyl)indole-3-carbonitrile (189), 1-(4-fluorophenyl)-5-hydroxy-2-(1-methoxypropyl)indole-3-carbonitrile (190) and 1-(4-fluorophenyl)-5-hydroxy-2-[(E)-prop-1-enyl]indole-3-carbonitrile (191)

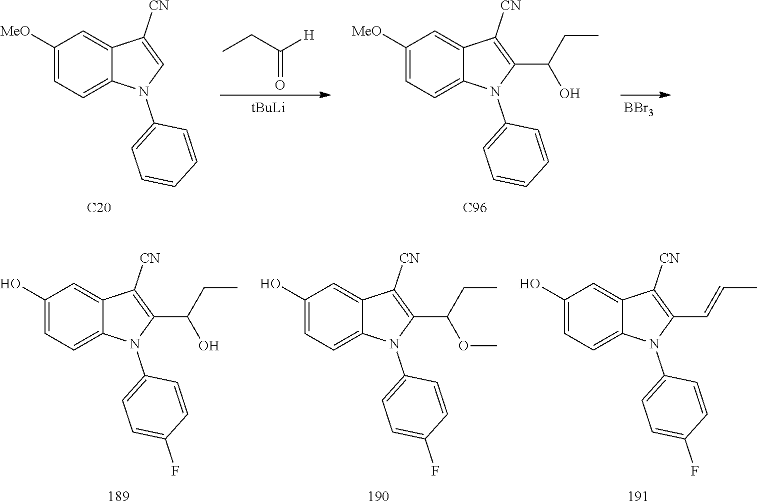

### Step 1. Synthesis of 1-(4-fluorophenyl)-2-(1-hydroxypropyl)-5-methoxy-1H-indole-3-carbonitrile (C96)

To a cold (−78° C.) solution of 1-(4-fluorophenyl)-5-methoxy-indole-3-carbonitrile (0.25 g, 0.93 mmol) in THF (6 mL) was added dropwise a solution of tert-butyllithium (0.64 mL of 1.7 M in pentane, 1.08 mmol). After 1 hour, the reaction mixture was added to propanal (0.07 mL, 0.93 mmol). The reaction mixture was kept at −78° C. for 1 hour, then warmed to room temperature. After 2 hours, the mixture was diluted into water and extracted three times with EtOAc. The organic phase was dried (MgSO₄), filtered, and concentrated in vacuo. The resulting residue was purified by silica gel chromatography (12 g ISCO column) using 0-40% EtOAc/heptanes gradient to afford 78 mg of product. 1-(4-fluorophenyl)-2-(1-hydroxypropyl)-5-methoxy-indole-3-carbonitrile (52%). ¹H NMR (400 MHz, Chloroform-d) δ 7.35-7.24 (m, 2H), 7.21-7.10 (m, 2H), 7.07 (t, J=1.5 Hz, 1H), 6.80 (d, J=1.5 Hz, 2H), 4.66-4.42 (m, 1H), 3.79 (s, 3H), 2.39 (d, J=6.1 Hz, 1H), 1.92-1.74 (m, 2H), 0.82 (t, J=7.4 Hz, 3H). ESI-MS m/z calc. 324.13, found 325.15 (M+1)⁺.

### Step 2. Synthesis of 1-(4-fluorophenyl)-5-hydroxy-2-(I-hydroxypropyl)indole-3-carbonitrile (189), 1-(4-fluorophenyl)-5-hydroxy-2-(I-methoxypropyl)indole-3-carbonitrile (190) and 1-(4-fluorophenyl)-5-hydroxy-2-[(E)-prop-1-enyl]indole-3-carbonitrile (191)

To a cold (−78° C.) solution of 1-(4-fluorophenyl)-2-(1-hydroxypropyl)-5-methoxy-indole-3-carbonitrile C95 (0.076 g, 0.230 mmol) in CH₂Cl₂ (10 mL) was added a solution of tribromoborane (0.70 mL of 1 M, 0.70 mmol) in CH₂Cl₂. After 90 minutes, the reaction temperature was raised to room temperature. After 2 hours, the mixture was kept at 4° C. for 2 days and then additional tribromoborane (0.30 mL of 1 M, 0.30 mmol) was added. The reaction mixture was stirred for 1 hour and diluted into water and extracted three times with CH₂Cl₂. The solvent was removed under reduced pressure. The crude residue was purified by reverse phase flash chromatography (RF ISCO, C18 column, 30 g) eluting with CH₃CN/water (0-100%, 0.1% TFA) to afford the desired products.

Product 189: 1-(4-fluorophenyl)-5-hydroxy-2-(1-hydroxypropyl)indole-3-carbonitrile (TFA salt) (7.2 mg, 7%). Racemic mixture. ¹H NMR (300 MHz, DMSO-d6) δ 9.35 (s, 1H), 7.71-7.41 (m, 4H), 6.95 (s, 1H), 6.89-6.65 (m, 2H), 5.75 (s, 1H), 4.46 (br. s, 1H), 1.86-1.49 (m, 2H), 0.76 (t, J=7.1 Hz, 3H). ESI-MS m/z calc. 310.11, found 311.15 (M+1)⁺.

Product 190: 1-(4-fluorophenyl)-5-hydroxy-2-(1-methoxypropyl)indole-3-carbonitrile (38 mg, 44%). Racemic mixture. ¹H NMR (300 MHz, Chloroform-d) δ 7.29 (q, J=8.9, 8.2 Hz, 5H), 6.87 (d, J=1.2 Hz, 2H), 4.31 (t, J=7.0 Hz, 1H), 3.36 (s, 3H), 1.88 (dt, J=14.7, 7.4 Hz, 1H), 1.72 (dq, J=13.9, 7.1 Hz, 1H), 0.89 (t, J=7.4 Hz, 3H). ESI-MS m/z calc. 324.13, found 325.15 (M+1)⁺.

Product 191: 1-(4-fluorophenyl)-5-hydroxy-2-[(E)-prop-1-enyl]indole-3-carbonitrile (5.5 mg, 8%). ¹H NMR (300 MHz, Chloroform-d) δ 7.36-7.21 (m, 5H), 7.00-6.67 (m, 3H), 6.23-6.03 (m, 1H), 1.92 (dd, J=6.8, 1.5 Hz, 3H). ESI-MS m/z calc. 292.10, found 293.15 (M+1)⁺.

### Compound 192

### 5-hydroxy-2-(I-hydroxypropyl)-1-(2-methylpyridin-4-yl)-1H-indole-3-carbonitrile (192)

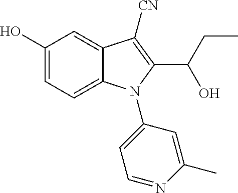 [SMILES: CCC(O)c1c(C#N)c2cc(O)ccc2n1-c1ccnc(C)c1]

Compound 192 was prepared in same fashion as 189 using 4-iodo-2-methyl pyridine instead of 4-fluoroiodobenzene as described in the synthesis of C20. Lithiation with tert-butyl lithium and alkylation with propanal was followed by boron tribromide removal of methyl protecting group on phenol. ¹H NMR (400 MHz, DMSO-d6) δ 8.70 (d, J=5.3 Hz, 1H), 7.44 (d, J=1.7 Hz, 1H), 7.37 (dd, J=5.3, 1.7 Hz, 1H), 6.96 (d, J=8.9 Hz, 1H), 6.94 (d, J=2.1 Hz, 1H), 6.77 (dd, J=8.9, 2.3 Hz, 1H), 6.05-5.61 (m, 1H), 4.54 (t, J=6.8 Hz, 1H), 2.59 (s, 3H), 1.84-1.65 (m, 2H), 0.76 (t, J=7.4 Hz, 3H). LCMS m/z 309.4 [M+H]⁺.

### Compound 193

### 6-fluoro-1-(4-fluoro-3-methylphenyl)-5-hydroxy-2-isopropyl-1H-indole-3-carbonitrile (193)

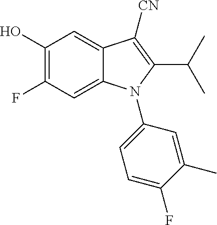 [SMILES: Cc1cc(-n2c(C(C)C)c(C#N)c3cc(O)c(F)cc32)ccc1F]

### Step 1. Synthesis of 6-fluoro-1-(4-fluoro-3-methylphenyl)-2-isopropyl-5-methoxy-1H-indole-3-carbonitrile (C97)

A solution of 6-fluoro-1-(4-fluoro-3-methyl-phenyl)-2-isopropyl-5-methoxy-indole-3-carbaldehyde C64 (0.55 g, 1.59 mmol) and NH₂OH—HCl (0.17 g, 2.39 mmol) in EtOH (20 mL) was heated at 85° C. for 3 hours. The solvent was removed under reduced pressure. The residue was dissolved in water (10 mL) and extracted three times with EtOAc. The organic phase was dried (MgSO₄), filtered, and concentrated in vacuo to afford 554 mg of product. 1-(3,4-difluorophenyl)-2-isopropyl-5-methoxy-6-methyl-indole-3-carbaldehyde oxime (97%).

To a solution of the product (554 mg) in dioxane (10.0 mL) and pyridine (2.0 mL, 24.73 mmol) was added methanesulfonyl chloride (0.74 g, 6.46 mmol). The reaction mixture was heated at 100° C. for 24 hours in a closed vial. The solvent was evaporated under reduced pressure. The residue was dissolved in water (100 mL) and extracted three times with EtOAc. The organic phase was dried (MgSO₄), filtered, and concentrated in vacuo. The resulting residue was purified by silica gel chromatography using 0-60% EtOAc/heptanes gradient to afford 280 mg of product. 6-fluoro-1-(4-fluoro-3-methyl-phenyl)-2-isopropyl-5-methoxy-indole-3-carbonitrile (50%). ¹H NMR (400 MHz, Chloroform-d) δ 7.26-7.20 (m, 2H), 7.17-7.07 (m, 2H), 6.70 (d, J=10.9 Hz, 1H), 3.98 (s, 3H), 3.04-2.83 (m, 1H), 2.40 (d, J=2.1 Hz, 3H), 1.45 (dd, J=7.1, 5.7 Hz, 6H). ESI-MS m/z calc. 340.14, found 341.22 (M+1)⁺.

### Step 2. Synthesis 6-fluoro-1-(4-fluoro-3-methylphenyl)-5-hydroxy-2-isopropyl-1H-indole-3-carbonitrile (193)

To a cold (0° C.) solution of 6-fluoro-1-(4-fluoro-3-methyl-phenyl)-2-isopropyl-5-methoxy-indole-3-carbonitrile C97 (0.117 g, 0.333 mmol) in CH₂Cl₂ (3 mL) was added tribromoborane (1.0 mL of 1 M, 1.0 mmol). The resulting solution was stirred at room temperature for 18 h. The reaction was quenched by addition of aqueous saturated NaHCO₃ solution. The aqueous phase was extracted three times with CH₂Cl₂. The combined organic phases were dried (MgSO₄), filtered, and concentrated in vacuo. The resulting residue was purified by silica gel chromatography using 0-70% EtOAc/heptanes gradient to afford 83 mg of product. 6-fluoro-1-(4-fluoro-3-methyl-phenyl)-5-hydroxy-2-isopropyl-indole-3-carbonitrile (70%). ¹H NMR (400 MHz, DMSO-d6) δ 9.79 (s, 1H), 7.50-7.34 (m, 3H), 7.09 (d, J=8.1 Hz, 1H), 6.77 (d, J=10.9 Hz, 1H), 2.88 (q, J=7.0 Hz, 1H), 2.32 (d, J=2.0 Hz, 3H), 1.34 (d, J=4.0 Hz, 3H). 1.32 (d, J=4.0 Hz, 3H). ESI-MS m/z calc. 326.12, found 327.19 (M+1)⁺.

### Compound 194

### 1-(4-fluorophenyl)-5-hydroxy-2-(tetrahydro-2H-pyran-4-yl)-1H-indole-3-carbonitrile (194)

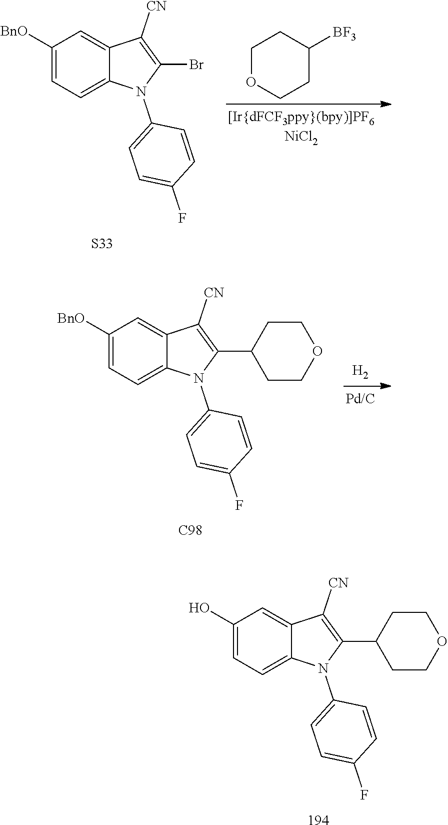

### Step 1. Synthesis of 5-(benzyloxy)-1-(4-fluorophenyl)-2-(tetrahydro-2H-pyran-4-yl)-1H-indole-3-carbonitrile (C98)

Dimethyl acetamide (1.0 mL) was added to dichloronickel; 1,2-dimethoxyethane (0.005 g, 0.023 mmol) and 4-tert-butyl-2-(4-tert-butyl-2-pyridyl)pyridine (0.006 g, 0.022 mmol). The vial was sealed with a rubber septa, the contents were stirred for five minutes and the solution turned green. 5-benzyloxy-2-bromo-1-(4-fluorophenyl)indole-3-carbonitrile S33 (0.082 g, 0.186 mmol), trifluoro(tetrahydropyran-4-yl)boranuide (Potassium Ion (1)) (0.053 g, 0.276 mmol), 2,6-lutidine (0.036 mL, 0.311 mmol), and [Ir{dFCF₃ ppy}(bpy)]PF₆ (Phosphorus Hexafluoride Ion) (0.005 g, 0.005 mmol) were added to the reaction mixture followed by 1,4-dioxane (4.0 mL) (anhydrous, sparged for 10 minutes with nitrogen prior to use). The contents were filtered into a second 10 mL vial sealed with a septa, evacuated, and filled with nitrogen gas. The entire solution was run through a Vaportech easy Medchem flow reactor at a flow rate of 0.25 mL min irradiating with Vaportech LED Gen 124 Watt @ 450 nm. (40 min residence time). The product was collected and the majority of the solvent was removed under reduced pressure. The resulting residue was purified by silica gel chromatography (40 g ISCO column) using 0-60% EtOAc/heptanes gradient to afford 36 mg of product. 5-benzyloxy-1-(4-fluorophenyl)-2-tetrahydropyran-4-yl-indole-3-carbonitrile (44%). ¹H NMR (400 MHz, Chloroform-d) δ 7.60-7.49 (m, 2H), 7.45-7.23 (m, 8H), 6.97 (d, J=2.4 Hz, 1H), 6.85 (dd, J=8.9, 0.6 Hz, 1H), 5.16 (s, 2H), 4.12-3.97 (m, 2H), 3.35-3.29 (m, 2H), 2.90-2.82 (m, 2H), 2.42-2.34 (m, 2H), 1.74-1.69 (m, 2H). ESI-MS m/z calc. 426.17, found 427.19 (M+1)⁺.

### Step 2. Synthesis of 5-(benzyloxy)-1-(4-fluorophenyl)-2-(tetrahydro-2H-pyran-4-yl)-1H-indole-3-carbonitrile (194)

To a solution of 5-benzyloxy-1-(4-fluorophenyl)-2-tetrahydropyran-4-yl-indole-3-carbonitrile C98 (0.036 g, 0.084 mmol) in MeOH (2.0 mL) and EtOAc (1.0 mL) purged with nitrogen was added Pd/C (0.100 g, 0.094 mmol). The reaction mixture was evacuated and purged with hydrogen and stirred under a hydrogen atmosphere for 2 hours. The crude mixture was filtered through a pad of celite and the filtrate was concentrated in vacuo. The resulting residue was purified by silica gel chromatography (40 g ISCO column) using 0-60% EtOAc/heptanes gradient to afford 18 mg of product. 1-(4-fluorophenyl)-5-hydroxy-2-tetrahydropyran-4-yl-indole-3-carbonitrile (61%). ESI-MS m/z calc. 336.13, found 337.11 (M+1)⁺. ¹H NMR (400 MHz, Chloroform-d) δ 7.33 (s, 2H), 7.31 (s, 2H), 7.22 (t, J=1.5 Hz, 1H), 6.82 (s, 2H), 4.12-3.95 (m, 2H), 3.32 (td, J=12.0, 1.9 Hz, 2H), 2.93-2.73 (m, 1H), 2.41-2.31 (m, 2H), 1.74-1.70 (m, 2H).

### Compounds 195-203

Compounds 195-203 (Table 15) were prepared from S33 or S32 using the method used for the preparation of compound 194. The appropriate alkyl boronate salt was used in the reaction.

TABLE 15
Structure and physicochemical data for compounds 195-203

| | | | ¹H NMR; LCMS m/z |
|---|---|---|---|
| Compound | Method/Product | Reagent | [M + H]⁺ |
| 195 | 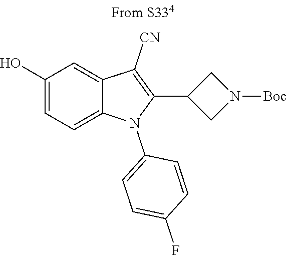 [SMILES: CC(C)(C)OC(=O)N1CC(c2c(C#N)c3cc(O)ccc3n2-c2ccc(F)cc2)C1] | 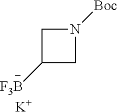 | ¹H NMR (300 MHz, Chloroform-d) δ 7.29 (s, 2H), 7.27 (s, 2H), 7.23 (dd, J = 2.1, 0.9 Hz, 1H), 6.92-6.82 (m, 2H), 6.37- 6.32(m, 1H), 4.35 (brs, 2H), 4.18-4.02 (brm, 2H), 4.02-3.86 (m, 1H), 1.46 (s, 9H). LCMS m/z 408.2 [M + H]⁺. |
| 196 | 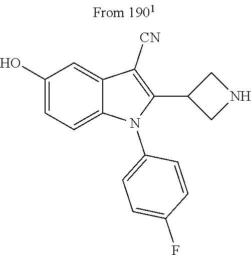 [SMILES: N#Cc1c(C2CNC2)n(-c2ccc(F)cc2)c2ccc(O)cc12] | | ¹H NMR (400 MHz, Methanol-d4) δ 7.59- 7.14 (m, 4H), 7.04 (s, 1H), 6.92 (d, J = 8.9 Hz, 1H), 6.83 (dd, J = 8.9, 2.4 Hz, 1H), 4.83- 4.49(m, 3H), 4.12-4.08 (m, 2H). LCMS m/z 308.2 [M + H]⁺. |
| 197 | 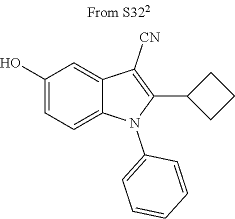 [SMILES: N#Cc1c(C2CCC2)n(-c2ccccc2)c2ccc(O)cc12] | 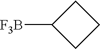 | ¹H NMR (300 MHz, Chloroform-d) δ 7.66- 7.49 (m, 3H), 7.36-7.26 (m, 3H), 7.24 (dd, J = 2.4, 0.6 Hz, 1H), 6.89 (dd, J = 8.8, 0.6 Hz, 1H), 6.79 (dd, J = 8.8, 2.4 Hz, 1H), 5.53 (s, 1H), 3.69- 3.50 (m, 1H), 2.75-2.52 (m, 2H), 2.17 (dddt, J = 13.6, 9.1, 6.5, 2.6 Hz, 2H), 2.03-1.83 (m, 2H). LCMS m/z 289.2 [M + H]⁺. |
| 198 | 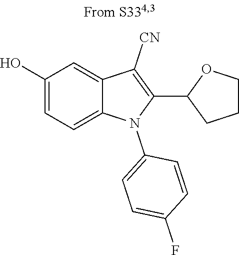 [SMILES: N#Cc1c(C2CCCO2)n(-c2ccc(F)cc2)c2ccc(O)cc12] | 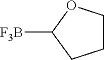 | ¹H NMR (400 MHz, Chloroform-d) δ 7.42- 7.34 (s, 2H), 7.33-7.20 (m, 3H), 6.91-6.76 (m, 2H), 4.98-4.70 (m, 1H), 4.25-4.10 (m, 1H), 3.95- 3.71 (m, 1H), 2.28- 2.09 (m, 3H), 2.06-1.88 (m, 1H). LCMS m/z 323.2 [M + H]⁺. |
| 199 | 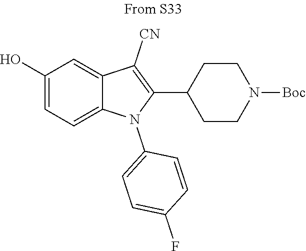 [SMILES: CC(C)(C)OC(=O)N1CCC(c2c(C#N)c3cc(O)ccc3n2-c2ccc(F)cc2)CC1] | 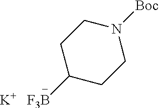 | ¹H NMR (400 MHz, Chloroform-d) δ 7.32 (s, 2H), 7.31 (s, 2H), 7.24- 7.15 (m, 1H), 6.85-6.70 (m, 2H), 4.24 (brs, 2H), 2.77-2.46 (m, 3H), 2.16 (s, 2H), 1.84-1.75 (m, 2H), 1.50 (s, 9H). LCMS m/z 436.2 [M + H]⁺. |
| 200 | 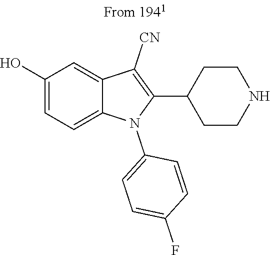 [SMILES: N#Cc1c(C2CCNCC2)n(-c2ccc(F)cc2)c2ccc(O)cc12] | | ¹H NMR (400 MHz, Methanol-d4) δ 7.51- 7.45 (m, 2H), 7.43-7.24 (m, 2H), 7.04-6.93 (m, 1H), 6.86-6.70 (m, 2H), 3.48-3.45 (m, 2H), 3.03- 2.94 (m, 3H), 2.45-2.35 (m, 2H), 2.18-2.14 (m, 2H). LCMS m/z 336.2 [M + H]⁺. |
| 201 | 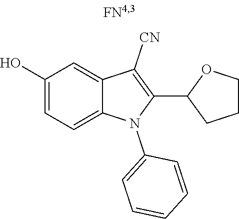 [SMILES: N#Cc1c(C2CCCO2)n(-c2ccccc2)c2ccc(O)cc12] | 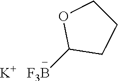 | ¹H NMR (400 MHz, Chloroform-d) δ 7.42- 7.34 (s, 2H), 7.33-7.20 (m, 3H), 6.91-6.76 (m, 2H), 4.98-4.70 (m, 1H), 4.25-4.10 (m, 1H), 3.95- 3.71 (m, 1H), 2.28- 2.09 (m, 3H), 2.06-1.88 (m, 1H). LCMS m/z 323.2 [M + H]⁺. |
| 202 | 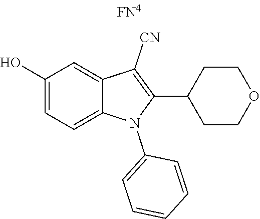 [SMILES: N#Cc1c(C2CCOCC2)n(-c2ccccc2)c2ccc(O)cc12] | 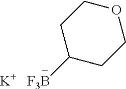 | ¹H NMR (300 MHz, Chloroform-d) δ 7.61- 7.46 (m, 3H), 7.30-7.21 (m, 2H), 7.15 (dd, J = 2.2, 0.8 Hz, 1H), 6.81- 6.62 (m, 2H), 5.50 (s, 1H), 4.06-3.87 (m, 2H), 3.22 (td, J = 11.9, 1.9 Hz, 2H), 2.80 (tt, J = 12.3, 3.6 Hz, 1H), 2.41-2.15 (m, 2H), 1.74-1.56 (m, 2H). LCMS m/z 319.1 [M + H]⁺. |
| 203 | 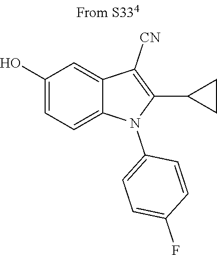 [SMILES: N#Cc1c(C2CC2)n(-c2ccc(F)cc2)c2ccc(O)cc12] | 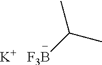 | ¹H NMR (400 MHz, Chloroform-d) δ 7.46- 7.37 (m, 2H), 7.35-7.27 (m, 2H), 7.18 (d, J = 2.4 Hz, 1H), 6.91 (d, J = 8.8 Hz, 1H), 6.81 (dd, J = 8.8, 2.4 Hz, 1H), 5.43 (s, 1H), 1.84-1.72 (m, 1H), 1.29-1.12 (m, 2H), 1.04- 0.96 (m, 2H). LCMS m/z 293.1 [M + H]⁺. |

¹Final deprotection step: HCl (4M solution), dioxane, room temperature.
²Final deprotection step: BBr₃, CH₂Cl₂
³Compound is a racemic mixture
⁴Final deprotection step: H₂, Pd/C, MeOH or EtOAc

### Compound 204

### 5-hydroxy-2-isopropyl-1-propyl-1H-indole-3-carbonitrile (204)

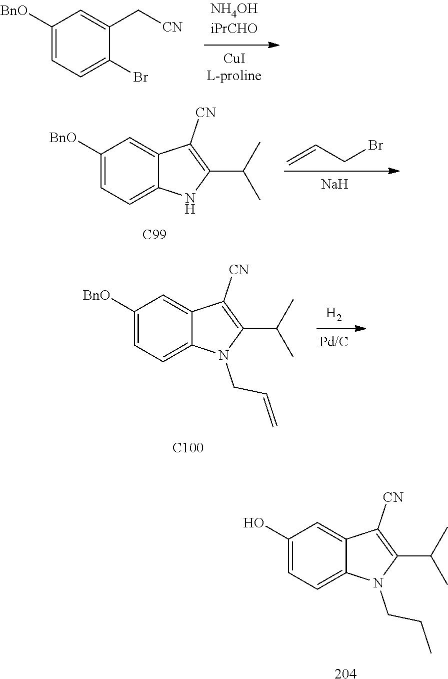

### Step 1. Synthesis of 5-(benzyloxy)-2-isopropyl-1H-indole-3-carbonitrile (C99)

To a suspension of 2-(5-benzyloxy-2-bromo-phenyl)acetonitrile (0.200 g, 0.546 mmol), potassium carbonate (0.080 g, 0.575 mmol), L-proline (0.026 g, 0.224 mmol), iodocopper (0.021 g, 0.110 mmol) in DMSO (2.084 mL) was added 2-methylpropanal (0.075 g, 1.033 mmol) and ammonium hydroxide (0.500 mL). The vial was sealed and irradiated in a microwave reactor at 100° C. After 20 hours, the mixture was cooled to room temperature and opened and then reheated to 100° C. for 6 hrs. The mixture was cooled to room temperature, diluted with water and extracted twice with EtOAc. The organic phases were washed twice with brine, dried over sodium sulfate, filtered and concentrated in vacuo. The resulting residue was purified by silica gel chromatography (12 g ISCO column) using 0-100% EtOAc/heptanes gradient to afford 140 mg of product. 5-benzyloxy-2-isopropyl-1H-indole-3-carbonitrile (75%). ¹H NMR (300 MHz, Chloroform-d) δ 8.29 (s, 1H), 7.53-7.29 (m, 6H), 7.30-7.23 (m, 2H), 7.23-7.16 (m, 1H), 6.96 (dd, J=8.8, 2.4 Hz, 1H), 3.38 (p, J=7.0 Hz, 1H), 1.44 (d, J=7.0 Hz, 7H). ESI-MS m/z calc. 290.14, found 291.15 (M+1)⁺.

### Step 2. Synthesis of I-allyl-5-(benzyloxy)-2-isopropyl-1H-indole-3-carbonitrile (C100)

To a flask containing 5-benzyloxy-2-isopropyl-1H-indole-3-carbonitrile C99 (0.097 g, 0.286 mmol) was added 1-methylpyrrolidin-2-one (1.0 mL) followed by 60% sodium hydride (0.047 g, 60% w/w, 1.175 mmol). To the reaction mixture was added dropwise 3-bromoprop-1-ene (0.070 g, 0.579 mmol). The resulting mixture was stirred at room temperature for 30 minutes, cooled to 0° C. and quenched with HCl (1.0 mL of 1 M, 1.000 mmol). The mixture was cooled to room temperature, diluted with water and extracted twice with EtOAc. The organic phases were washed twice with brine, dried over sodium sulfate, filtered and concentrated in vacuo. (134 mg). The resulting residue was purified by silica gel chromatography (12 g ISCO column) using 0-100% EtOAc/heptanes gradient to afford 80 mg of product. 1-allyl-5-benzyloxy-2-isopropyl-indole-3-carbonitrile (82%). ESI-MS m/z calc. 330.17, found 331.16 (M+1)⁺,

### Step 3. Synthesis of 5-hydroxy-2-isopropyl-1-propyl-1H-indole-3-carbonitrile (204)

To a solution of 1-allyl-5-benzyloxy-2-isopropyl-indole-3-carbonitrile C100 (0.081 g, 0.234 mmol) in MeOH (50 mL) was added Pd/C (0.098 mg of 10%). The reaction mixture was evacuated and purged with hydrogen and stirred under a hydrogen atmosphere for 1 hour. The crude mixture was filtered through a pad of celite and the filtrate was concentrated in vacuo. The resulting residue was purified by silica gel chromatography (4 g ISCO column) using 0-100% EtOAc/heptanes gradient to afford 51 mg of product. 5-hydroxy-2-isopropyl-1-propyl-indole-3-carbonitrile (89%). ¹H NMR (300 MHz, Chloroform-d) δ 7.31-7.12 (m, 3H), 6.86 (dd, J=8.8, 2.4 Hz, 1H), 6.05 (s, 1H), 4.13-3.98 (m, 2H), 3.22 (p, J=7.0 Hz, 1H), 1.78 (dt, J=15.0, 7.5 Hz, 2H), 1.53 (d, J=7.0 Hz, 7H), 0.98 (t, J=7.4 Hz, 3H). ESI-MS m/z calc. 242.14, found 243.12 (M+1)⁺.

### Compound 205

### 2-(tert-butyl)-5-hydroxy-1-propyl-1H-indole-3-carbonitrile (205)

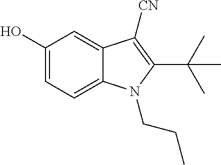 [SMILES: CCCn1c(C(C)(C)C)c(C#N)c2cc(O)ccc21]

Compound 205 was prepared from 2-(5-benzyloxy-2-bromo-phenyl)acetonitrile by copper-mediated coupling with pivaldehyde and cyclization as described for C99 in the preparation of compound 204. ¹H NMR (300 MHz, Chloroform-d) δ 7.22-7.07 (m, 2H), 6.86 (dd, J=8.8, 2.5 Hz, 1H), 5.48 (s, 1H), 4.29-4.09 (m, 2H), 1.98-1.75 (m, 2H), 1.62 (s, 9H), 1.06 (t, J=7.5 Hz, 3H). LCMS m/z 257.2 [M+H]⁺.

### Compound 206

### 1-(3-cyano-1-(4-fluorophenyl)-5-hydroxy-1H-indol-2-yl)-S-methylmethanesulfinamide (206)

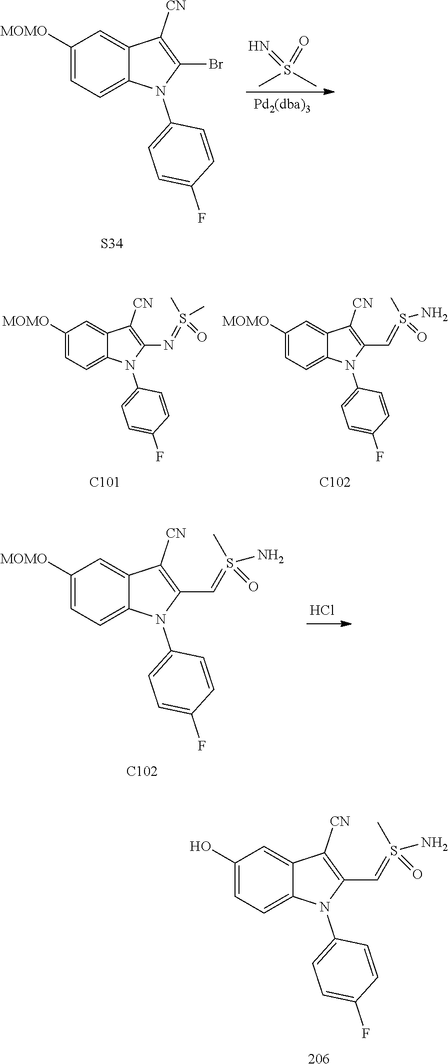

### Step 1. Synthesis of 1-(3-cyano-1-(4-fluorophenyl)-5-(methoxymethoxy)-1H-indol-2-yl)-S-methylmethanesulfinamide (C102)

To a solution of 2-bromo-1-(4-fluorophenyl)-5-(methoxymethoxy)indole-3-carbonitrile S34 (0.206 g, 0.549 mmol) in 1,4-dioxane (6.0 mL) was added imino-dimethyl-oxo-Î>>6-sulfane (0.066 g, 0.708 mmol). The mixture was bubbled with nitrogen gas for 5 minutes and Pd₂(dba)₃ (0.027 g, 0.029 mmol), Xantphos (0.031 g, 0.053 mmol) and Cs₂CO₃ (0.315 g, 0.967 mmol) were added. The vial was sealed and irradiated in a microwave reactor at 120° C. for 17 hours. The mixture was then filtered through celite and concentrated onto silica gel. The resulting residue was purified by silica gel chromatography (12 g ISCO column) using 0-100% EtOAc/heptanes gradient to afford two products. 64 mg of 2-[[dimethyl (oxo)-Î>>6-sulfanylidene]amino]-1-(4-fluorophenyl)-5-(methoxymethoxy)indole-3-carbonitrile C101 (27%). ¹H NMR (300 MHz, Chloroform-d) δ 7.47-7.35 (m, 2H), 7.33-7.30 (m, 1H), 7.27-7.13 (m, 2H), 6.98-6.92 (m, 1H), 6.89 (dd, J=8.8, 2.3 Hz, 1H), 5.22 (s, 2H), 3.53 (s, 3H), 3.27 (s, 6H). ESI-MS m/z calc. 387.1, found 388.1 (M+1)⁺. 61 mg of 2-[(amino-methyl-oxo-Î>>6-sulfanylidene)methyl]-1-(4-fluorophenyl)-5-(methoxymethoxy) indole-3-carbonitrile C102 (25%). ¹H NMR (300 MHz, Chloroform-d) δ 7.59-7.41 (m, 2H), 7.30-7.13 (m, 3H), 7.05 (d, J=8.7 Hz, 1H), 6.85 (dd, J=8.7, 2.3 Hz, 1H), 5.22 (s, 2H), 4.95 (s, 1H), 4.77 (s, 2H), 3.54 (s, 3H), 3.30 (s, 3H). ESI-MS m/z calc. 387.1, found 388.1 (M+1)⁺. ¹⁹F NMR (282 MHz, Chloroform-d) 6-113.76. ¹³C NMR (75 MHz, Chloroform-d) δ 163.32 (s), 160.05 (s), 152.93 (s), 152.79 (s), 151.71 (s), 131.65 (s), 131.07 (s), 129.30 (d), 129.18 (d), 123.50 (s), 116.38 (d), 116.08 (d), 110.46 (d), 110.15 (d), 105.55 (d), 95.76 (t), 86.60 (s), 71.04 (d), 55.98 (t), 49.51 (t).

### Step 2. Synthesis of 1-(3-cyano-1-(4-fluorophenyl)-5-hydroxy-1H-indol-2-yl)-S-methylmethanesulfinamide (206)

A suspension of 1-(3-cyano-1-(4-fluorophenyl)-5-(methoxymethoxy)-1H-indol-2-yl)-S-methylmethanesulfinamide C102 (0.045 g, 0.095 mmol) in MeOH (3 mL) was treated with hydrogen chloride (0.300 mL of 12 M aqueous solution, 3.600 mmol) at 50° C. for 2 hours. The reaction mixture was evaporated. The resulting residue was purified by silica gel chromatography (4 g ISCO column) using 0-10% MeOH/CH₂Cl₂ gradient to afford 22 mg of product. 1-(3-cyano-1-(4-fluorophenyl)-5-hydroxy-1H-indol-2-yl)-S-methylmethanesulfinamide (46%). ¹H NMR (300 MHz, DMSO-d6) δ 8.79 (s, 1H), 7.61-7.44 (m, 2H), 7.44-7.32 (m, 2H), 7.25 (d, J=2.2 Hz, 1H), 6.82 (d, J=8.5 Hz, 1H), 6.51 (dd, J=8.6, 2.2 Hz, 1H), 6.33 (s, 2H), 4.98 (s, 1H), 3.31 (s, 3H), 3.28 (s, 3H). ESI-MS m/z calc. 343.1, found 344.1 (M+1)⁺.

### Compound 207

### 7-amino-1-(4-fluoro-3-methyl-phenyl)-5-hydroxy-2-methyl-indole-3-carbonitrile (207)

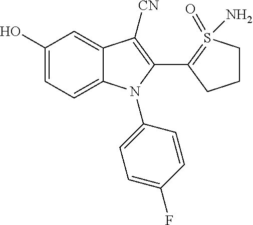 [SMILES: N#Cc1c(C2=S(N)(=O)CCC2)n(-c2ccc(F)cc2)c2ccc(O)cc12]

Compound 207 was prepared from 2-bromo-1-(4-fluorophenyl)-5-(methoxymethoxy)indole-3-carbonitrile S34 by palladium coupling with 1-iminotetrahydro-1H-1λ⁶-thiophene-1-oxide as described for C101 in the preparation of compound 206. ¹H NMR (300 MHz, DMSO-d6) δ 8.79 (s, 1H), 7.63-7.45 (m, 2H), 7.44-7.30 (m, 2H), 7.26 (d, J=2.2 Hz, 1H), 6.87 (d, J=8.6 Hz, 1H), 6.51 (dd, J=8.6, 2.3 Hz, 1H), 6.01 (s, 2H), 3.56 (dt, J=11.8, 4.8 Hz, 1H), 3.41-3.24 (m, 1H), 2.83 (dd, J=7.9, 5.7 Hz, 2H), 2.33-2.15 (m, 2H). LCMS m/z 370.0 [M+H]⁺.

### Compound 208

### 1-(4-fluoro-3-methylphenyl)-5-hydroxy-2,7-dimethyl-1H-indole-3-carbonitrile (208)

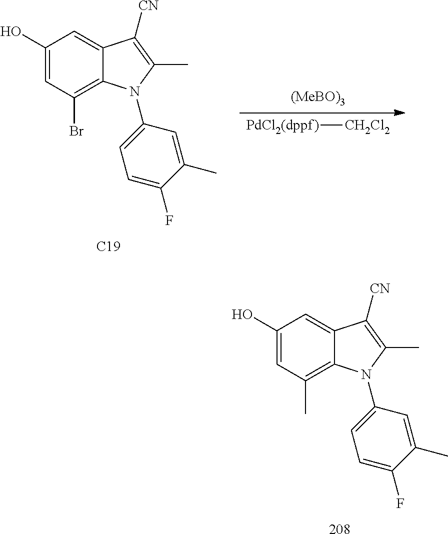

A mixture of 7-bromo-1-(4-fluoro-3-methyl-phenyl)-5-hydroxy-2-methyl-indole-3-carbonitrile C19 (0.050 g, 0.139 mmol), 2,4,6-trimethyl-1,3,5,2,4,6-trioxatriborinane (0.033 mL, 0.236 mmol), PdCl₂(dppf)·CH₂C₂ (0.009 g, 0.010 mmol) and K₂C₃ (0.058 g, 0.420 mmol) in dioxane (0.550 mL) was heated to 90° C. for 1 hour. The mixture was cooled to room temperature and diluted into water and EtOAc. Combined organic phases were washed with brine, dried (MgSO₄), filtered, and concentrated in vacuo. The resulting residue was purified by silica gel chromatography (12 g ISCO column) using 0-25%0 EtOAc/heptanes gradient to afford 27 mg of product. 1-(4-fluoro-3-methyl-phenyl)-5-hydroxy-2,7-dimethyl-indole-3-carbonitrile (65%2). ¹H NMR (400 MHz, DMSO-d6) δ9.18 (s, 1H), 7.51-7.41 (m, 1H), 7.41-7.29 (m, 2H), 6.74 (dd, J=2.4, 0.7 Hz, 1H), 6.47 (dd, J=2.4, 0.9 Hz, 1H), 2.30 (d, J 2.0 Hz, 3H), 2.21 (s, 3H), 1.74 (s, 3H). ESI-MS m/z calc. 294.1, found 295.2 (M+1)₃.

### Compounds 209 and 210

TABLE 16
Structure and physicochemical data for compounds 209-210

| | | | ¹H NMR; LCMS m/z |
|---|---|---|---|
| Compound | Method/Product | Reagent | [M + H]⁺ |
| 209 | 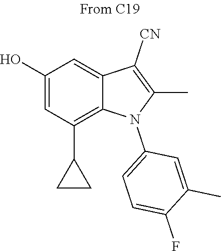 | | ¹H NMR (300 MHz, DMSO-d6) δ 8.79 (s, 1H), 7.63-7.45 (m, 2H), 7.44- 7.30 (m, 2H), 7.26 (d, J = 2.2 Hz, 1H), 6.87 (d, J = 8.6 Hz, 1H), 6.51 (dd, J = 8.6, 2.3 Hz, 1H), 6.01 (s, 2H), 3.56 (dt, J = 11.8, 4.8 Hz, 1H), 3.41-3.24 (m, 1H), 2.83 (dd, J = 7.9, 5.7 Hz, 2H), 2.33-2.15 (m, 2H). LCMS m/z 370.0 [M + H]⁺. |
| 210 | 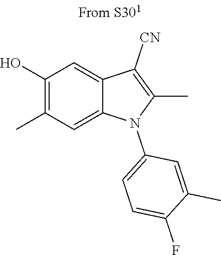 [SMILES: Cc1cc2c(cc1O)c(C#N)c(C)n2-c1ccc(F)c(C)c1] | (MeBO)₃ | ¹H NMR (400 MHz, DMSO-d6) δ 9.34 (s, 1H), 7.48-7.43 (m, 1H), 7.43- 7.28 (m, 2H), 6.93 (s, 1H), 6.80 (d, J = 0.8 Hz, 1H), 2.33 (s, 3H), 2.32 (d, J = 2.0 Hz, 3H), 2.15 (s, 3H). LCMS m/z 295.2 [M + H]⁺. |

¹Final deprotection step: H₂, Pd/C, EtOAc

### Compound 211

### 6-amino-1-(4-fluoro-3-methylphenyl)-5-hydroxy-2-methyl-1H-indole-3-carbonitrile (211)

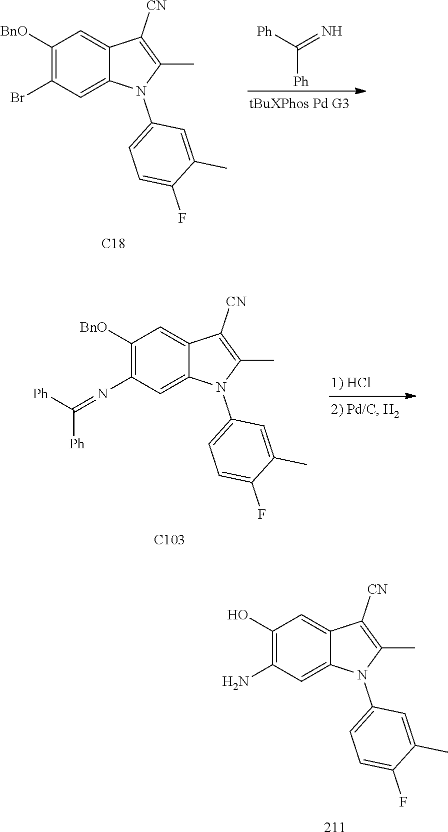

### Step 1: Synthesis of 5-(benzyloxy)-6-((diphenylmethylene)amino)-1-(4-fluoro-3-methylphenyl)-2-methyl-1H-indole-3-carbonitrile (C103)

A mixture of 5-benzyloxy-6-bromo-1-(4-fluoro-3-methyl-phenyl)-2-methyl-indole-3-carbonitrile C18 (0.100 g, 0.223 mmol), diphenylmethanimine (0.041 mL, 0.245 mmol), Xantphos Pd G3 (0.010 g, 0.011 mmol) and Cs₂CO₃ (0.218 g, 0.669 mmol) in dioxane (1.0 mL) was heated at 100° C. overnight. Additional diphenylmethanimine (0.041 mL, 0.245 mmol), Xantphos Pd G3 (0.010 g, 0.011 mmol) and Cs₂CO₃ (0.218 g, 0.669 mmol) were added and the mixture was heated at 100° C. for 6.5 hours. The mixture was cooled to room temperature filtered and concentrated in vacuo. The resulting residue was purified by silica gel chromatography (40 g ISCO column) using 0-60% EtOAc/heptanes gradient to afford 83 mg of product. 6-(benzhydrylideneamino)-5-benzyloxy-1-(4-fluoro-3-methyl-phenyl)-2-methyl-indole-3-carbonitrile (64%). ESI-MS m/z calc. 549.22, found 550.31 (M+1)⁺.

### Step 2: Synthesis of 6-amino-1-(4-fluoro-3-methylphenyl)-5-hydroxy-2-methyl-1H-indole-3-carbonitrile (211)

To a solution of 6-(benzhydrylideneamino)-5-benzyloxy-1-(4-fluoro-3-methyl-phenyl)-2-methyl-indole-3-carbonitrile C103 (0.083 g, 0.151 mmol) in THE (0.600 mL) was added HCl (0.250 mL of 2 M, 0.500 mmol). The reaction mixture was stirred at room temperature for 1 hour and the mixture was concentrated in vacuo. The residue was diluted with EtOAc, concentrated in vacuo, and triturated with heptanes to afford 58 mg of product. 6-amino-5-benzyloxy-1-(4-fluoro-3-methyl-phenyl)-2-methyl-indole-3-carbonitrile (Hydrochloride salt) (87%). ¹H NMR (400 MHz, DMSO-d6) δ 7.67-7.56 (m, 2H), 7.54-7.25 (m, 7H), 7.06 (s, 1H), 5.33 (s, 2H), 2.38 (s, 3H), 2.33 (d, J=1.9 Hz, 3H). ESI-MS m/z calc. 385.16, found 386.24 (M+1)⁺.

A suspension of the deprotected aniline 6-amino-5-benzyloxy-1-(4-fluoro-3-methyl-phenyl)-2-methyl-indole-3-carbonitrile (Hydrochloride salt) (0.021 g, 0.048 mmol) and Pd/C (0.006 g of 10% w/w, 0.006 mmol) in MeOH (1 mL) was stirred under an atmosphere of hydrogen for 1.5 hours. The crude mixture was filtered through a pad of celite and the filtrate was concentrated in vacuo. The crude residue was purified by reverse phase flash chromatography (RF ISCO, C₁₈ column, 30 g) eluting with CH₃CN/water (0-100%, 0.1% TFA). Fractions with desired product were neutralized with aqueous saturated NaHCO₃ solution and extracted with CH₂Cl₂. Combined organic extracts were passed through a phase separator and concentrated in vacuo to afford 4.7 mg of product. 6-amino-1-(4-fluoro-3-methyl-phenyl)-5-hydroxy-2-methyl-indole-3-carbonitrile (32%). ¹H NMR (400 MHz, Chloroform-d) δ 7.25-7.03 (m, 5H), 6.38 (s, 1H), 2.36 (s, 8H). ESI-MS m/z calc. 295.11, found 295.55 (M+1)⁺.

### Compound 212

### 7-amino-1-(4-fluoro-3-methyl-phenyl)-5-hydroxy-2-methyl-indole-3-carbonitrile (212)

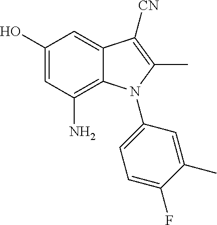 [SMILES: Cc1cc(-n2c(C)c(C#N)c3cc(O)cc(N)c32)ccc1F]

Compound 212 was prepared from 5-benzyloxy-7-bromo-1-(4-fluoro-3-methyl-phenyl)-2-methyl-indole-3-carbonitrile S31 by palladium coupling with diphenylmethanimine as described for C103 in the preparation of compound 211. ¹H NMR (400 MHz, DMSO-d6) δ 8.93 (s, 1H), 7.52-7.41 (m, 1H), 7.41-7.31 (m, 2H), 6.19 (d, J=2.2 Hz, 1H), 6.01 (d, J=2.2 Hz, 1H), 4.05 (s, 2H), 2.30 (d, J=1.5 Hz, 3H), 2.17 (s, 3H). ESI-MS m/z calc. 295.11, found 295.37 (M+1)⁺.

### Compound 213

### 3-(1-(4-fluoro-3-methylphenyl)-5-hydroxy-2-isopropyl-1H-indol-3-yl)-N-methylazetidine-1-sulfonamide (213)

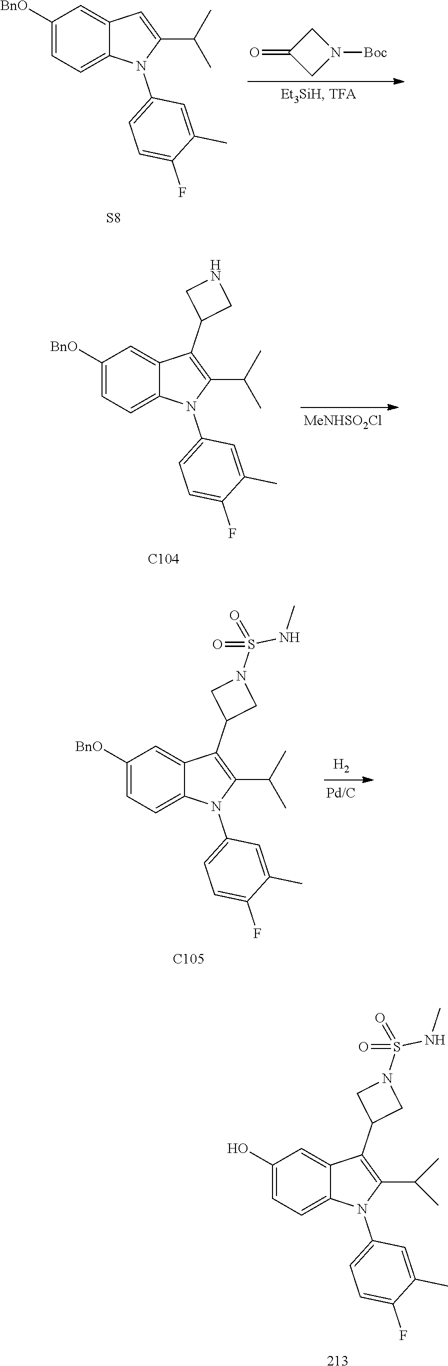

### Step 1. Synthesis of 3-(azetidin-3-yl)-5-(benzyloxy)-1-(4-fluoro-3-methylphenyl)-2-isopropyl-1H-indole (C104)

To a solution of 5-benzyloxy-1-(4-fluoro-3-methyl-phenyl)-2-isopropyl-indole S8 (0.72 g, 1.92 mmol) and tert-butyl 3-oxoazetidine-1-carboxylate (0.66 g, 3.83 mmol) in CH₂Cl₂ (12.8 mL) at 25° C. was added triethylsilane (1.80 mL, 11.27 mmol) followed by 2,2,2-trifluoroacetic acid (0.59 mL, 7.67 mmol). The mixture was heated to 50° C. and stirred for 3 days. The reaction mixture was evaporated to dryness, then was diluted with CH₂Cl₂ (5 mL) and HCl (9.6 mL of 4 M in dioxane, 38.40 mmol) was slowly added. The reaction was stirred 1 hour at room temperature and was evaporated to dryness. The residue was neutralized with aqueous saturated NaHCO₃ solution and the resulting aqueous phase was extracted three times with CH₂Cl₂. The organic phase was dried (MgSO₄), filtered, and concentrated in vacuo. The resulting residue was purified by silica gel chromatography (40 g ISCO column) using 0-50% MeOH/CH₂Cl₂ gradient to afford 136 mg of product. 3-(azetidin-3-yl)-5-benzyloxy-1-(4-fluoro-3-methyl-phenyl)-2-isopropyl-indole (13%). ¹H NMR (400 MHz, Chloroform-d) δ 7.63 (d, J=2.3 Hz, 1H), 7.56-7.47 (m, 2H), 7.41-7.35 (m, 2H), 7.34-7.28 (m, 1H), 7.17-7.00 (m, 3H), 6.84 (dd, J=8.9, 2.3 Hz, 1H), 6.78 (dd, J=8.9, 0.5 Hz, 1H), 5.20 (s, 2H), 4.57-4.48 (m, 1H), 4.43 (t, J=8.3 Hz, 2H), 3.95 (t, J=8.1 Hz, 2H), 2.98-2.86 (m, 1H), 2.32 (d, J=2.0 Hz, 3H), 1.24 (dd, J=7.3, 1.7 Hz, 6H). ESI-MS m/z calc. 428.2, found 428.0.

### Step 2. Synthesis of 3-(5-(benzyloxy)-1-(4-fluoro-3-methylphenyl)-2-isopropyl-1H-indol-3-yl)-N-methylazetidine-1-sulfonamide (C105)

To a solution of 3-(azetidin-3-yl)-5-(benzyloxy)-1-(4-fluoro-3-methylphenyl)-2-isopropyl-1H-indole C104 (0.040 g, 0.093 mmol) in CH₂Cl₂ was added triethylamine (0.014 mL, 0.103 mmol) followed by N-methylsulfamoyl chloride (0.014 g, 0.103 mmol). The reaction mixture was stirred at room temperature overnight. The solvent was removed under reduced pressure. The resulting residue was purified by silica gel chromatography using 0-100% EtOAc/heptanes gradient to afford 49 mg of product. 3-(5-(benzyloxy)-1-(4-fluoro-3-methylphenyl)-2-isopropyl-1H-indol-3-yl)-N-methylazetidine-1-sulfonamide (59%). ESI-MS m/z calculated 521.8, found 522.8 (M+1)⁺.

### Step 3. Synthesis of 3-(1-(4-fluoro-3-methylphenyl)-5-hydroxy-2-isopropyl-1H-indol-3-yl)-N-methylazetidine-1-sulfonamide (213)

A solution of -[5-benzyloxy-1-(4-fluoro-3-methyl-phenyl)-2-isopropyl-indol-3-yl]-N-methyl-azetidine-1-sulfonamide C105 (0.029 g, 0.056 mmol) in EtOAc (5 mL) was purged with nitrogen. Pd/C (0.006 g, 0.006 mmol, wet, Degussa) The reaction mixture was evacuated and purged with hydrogen and stirred under a hydrogen atmosphere for 30 minutes. The crude mixture was filtered through a pad of celite and the filtrate was concentrated in vacuo. The resulting residue was purified by silica gel chromatography using 0-100% EtOAc/heptanes gradient to afford 19 mg of product. 3-[1-(4-fluoro-3-methyl-phenyl)-5-hydroxy-2-isopropyl-indol-3-yl]-N-methyl-azetidine-1-sulfonamide (77%). ¹H NMR (400 MHz, Chloroform-d) δ 7.64 (s, 1H), 7.18-7.01 (m, 3H), 6.82-6.65 (m, 2H), 5.68 (s, 1H), 4.59-4.54 (m, 2H), 4.40-4.25 (m, 1H), 4.17-4.10 (m, 2H), 2.97-2.90 (m, 1H), 2.88 (d, J=5.3 Hz, 3H), 2.33 (s, 3H), 1.29-1.22 (m, 6H) ESI-MS m/z calc. 431.18, found 431.19 (M+1)⁺.

### Compound 214

### N-((3-(1-(4-fluoro-3-methylphenyl)-5-hydroxy-2-isopropyl-1H-indol-3-yl)azetidin-1-yl)sulfonyl)acetamide (214)

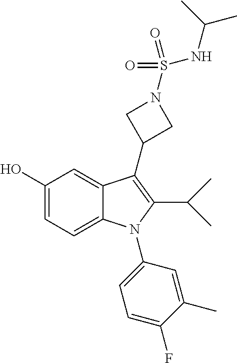 [SMILES: Cc1cc(-n2c(C(C)C)c(C3CN(S(=O)(=O)NC(C)C)C3)c3cc(O)ccc32)ccc1F]

Compound 214 was prepared from 3-(azetidin-3-yl)-5-(benzyloxy)-1-(4-fluoro-3-methylphenyl)-2-isopropyl-1H-indole using N-acetylsulfamoyl chloride as described for C105 in the preparation of 213. Hydrogenation with Pd/C in EtOAc afforded final product. ¹H NMR (400 MHz, Chloroform-d) δ 7.95 (s, 1H), 7.53 (s, 1H), 7.16-6.97 (m, 3H), 6.80-6.67 (m, 2H), 5.54 (s, 1H), 4.62-4.55 (m, 2H), 4.54-4.44 (m, 2H), 4.35-4.23 (m, 1H), 2.97-2.86 (m, 1H), 2.36-2.28 (m, 6H), 1.25-1.19 (m, 6H). ESI-MS m/z calc. 459.16, found 459.94 (M+1)⁺.

### Compound 215

### 3-[1-(4-fluoro-3-methyl-phenyl)-5-hydroxy-2-isopropyl-indol-3-yl]cyclobutanecarbonitrile (215)

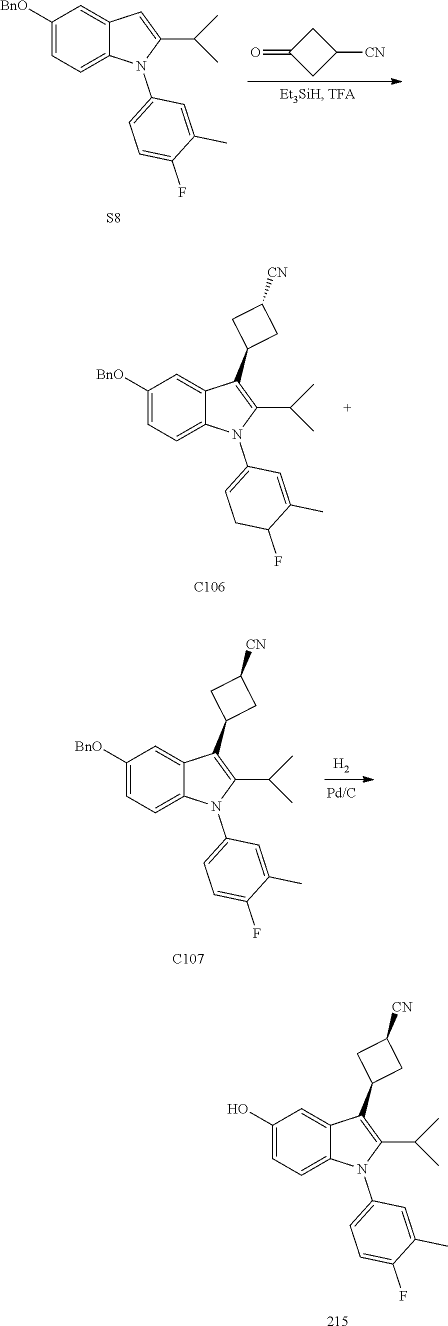

### Step 1. Synthesis of trans-3-(5-(benzyloxy)-1-(4-fluoro-3-methylphenyl)-2-isopropyl-1H-indol-3-yl)cyclobutane-1-carbonitrile (C106) and cis-3-(5-(benzyloxy)-1-(4-fluoro-3-methylphenyl)-2-isopropyl-1H-indol-3-yl)cyclobutane-1-carbonitrile (C107)

To a solution of 5-benzyloxy-1-(4-fluoro-3-methyl-phenyl)-2-isopropyl-indole S8 (0.50 g, 1.34 mmol), 3-oxocyclobutanecarbonitrile (0.26 g, 2.68 mmol) and triethylsilane (0.65 mL, 4.01 mmol) in CH₂Cl₂ (10 mL) was added trifluoroacetic acid (0.21 mL, 2.67 mmol). The solution was heated at 50° C. for 18 hours. The mixture was diluted with CH₂Cl₂ (10 mL) and washed water. The organic layer was separated, dried (MgSO₄), filtered and the solvent was removed under reduced pressure. The resulting residue was purified by silica gel chromatography (40 g ISCO column) using 0-50% EtOAc/heptanes gradient to afford 200 mg of product. 3-[5-benzyloxy-1-(4-fluoro-3-methyl-phenyl)-2-isopropyl-indol-3-yl]cyclobutanecarbonitrile. ESI-MS m/z calc. 452.2, found 452.1 (M+1)⁺.

### Step 2. Synthesis of 3-[1-(4-fluoro-3-methyl-phenyl)-5-hydroxy-2-isopropyl-indol-3-yl]cyclobutanecarbonitrile (215)

A solution of 3-[5-benzyloxy-1-(4-fluoro-3-methyl-phenyl)-2-isopropyl-indol-3-yl]cyclobutene-carbonitrile C107 (0.200 g, 0.439 mmol) in MeOH (5 mL) and EtOAc (5 mL) was purged with nitrogen. Pd/C (0.060 g, 0.056 mmol, wet, Degussa) The reaction mixture was evacuated and purged with hydrogen and stirred under a hydrogen atmosphere for 2 hours. The crude mixture was filtered through a pad of celite and the filtrate was concentrated in vacuo. The resulting residue was purified by silica gel chromatography (12 g ISCO column) using 0-70% EtOAc/heptanes gradient to afford 110 mg of product. 3-[1-(4-fluoro-3-methyl-phenyl)-5-hydroxy-2-isopropyl-indol-3-yl]cyclobutanecarbonitrile (67%). ¹H NMR (400 MHz, DMSO-d6) δ 8.90 (s, 1H), 7.39-7.28 (m, 2H), 7.22 (d, J=2.1 Hz, 1H), 7.18-7.16 (m, 2H), 6.69-6.08 (m, 2H), 4.09-3.84 (m, 1H), 3.48-3.34 (m, 1H), 2.95-2.79 (m, 3H), 2.66-2.62 (m, 2H), 2.29 (d, J=1.9 Hz, 3H), 1.21 (d, J=7.2 Hz, 6H). ESI-MS m/z calc. 362.18 found 361.44 (M+1)⁺.

### Compound 216

### N-((3-(1-(4-fluoro-3-methylphenyl)-5-hydroxy-2-isopropyl-1H-indol-3-yl)azetidin-1-yl)sulfonyl)acetamide (216)

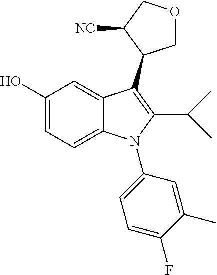 [SMILES: Cc1cc(-n2c(C(C)C)c([C@@H]3COC[C@@H]3C#N)c3cc(O)ccc32)ccc1F]

Compound 216 was prepared from 5-benzyloxy-1-(4-fluoro-3-methyl-phenyl)-2-isopropyl-indole (S8) using 4-oxotetrahydrofuran-3-carbonitrile as described in the preparation of C109. Hydrogenation with Pd/C in EtOAc afforded final product. ¹H NMR (400 MHz, Chloroform-d) δ 7.013-6.99 (m, 4H), 6.67-6.50 (m, 2H), 4.62 (s, 1H), 4.49 (dq, J=5.0, 3.1, 2.4 Hz, 1H), 4.37-4.18 (m, 2H), 4.18-4.00 (m, 2H), 3.48 (q, J=7.7 Hz, 1H), 2.99 (p, J=6.8 Hz, 1H), 2.26 (s, 3H), 1.24 (dd, J=7.3, 2.7 Hz, 3H), 1.17 (d, J=7.1 Hz, 3H). ESI-MS m/z found 379.7 (M+1)⁺.

### Compound 217

### 1-(4-fluoro-3-methyl-phenyl)-2-isopropyl-3-[3-(2H-tetrazol-5-yl)cyclobutyl]indol-5-ol (217)

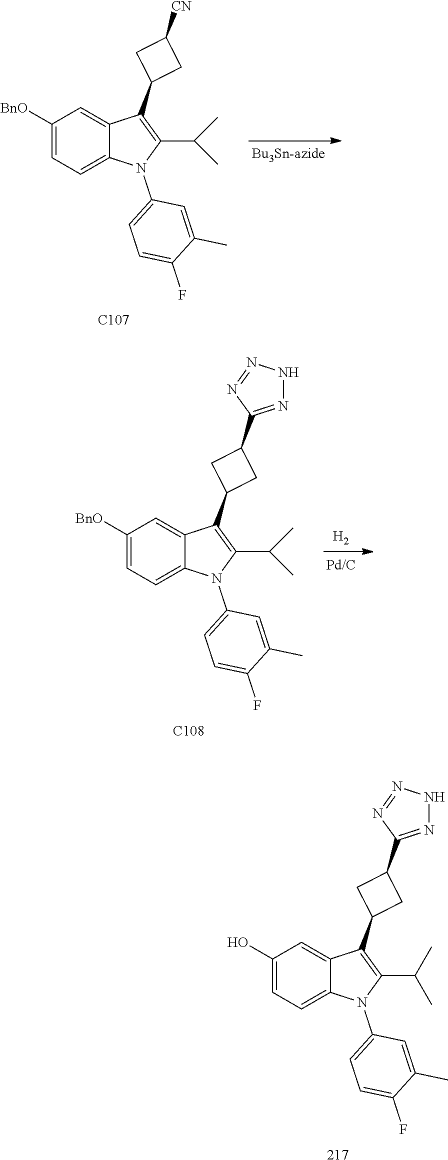

### Step 1. Synthesis of 3-((1s,3s)-3-(2H-tetrazol-5-yl)cyclobutyl)-5-(benzyloxy)-1-(4-fluoro-3-methylphenyl)-2-isopropyl-1H-indole (C108)

A solution of azido(tributyl)stannane (0.26 g, 0.78 mmol) and 3-[5-benzyloxy-1-(4-fluoro-3-methyl-phenyl)-2-isopropyl-indol-3-yl]cyclobutanecarbonitrile C107 (0.18 g, 0.39 mmol) in m-xylene (10 mL) was heated at 180° C. for 18 hour and then cooled to room temperature. The reaction mixture was diluted into water (10 mL) and EtOAc (10 mL). The organic phase was dried (MgSO₄), filtered, and concentrated in vacuo. The resulting residue was purified by silica gel chromatography using 0-60% EtOAc/heptanes gradient to afford 130 mg of product. 5-benzyloxy-1-(4-fluoro-3-methyl-phenyl)-2-isopropyl-3-[3-(2H-tetrazol-5-yl)cyclobutyl]indole (65%). ¹H NMR (400 MHz, Chloroform-d) δ 7.85 (s, 1H), 7.61-7.54 (m, 2H), 7.45-7.35 (m, 2H), 7.29 (s, 1H), 7.18-7.03 (m, 3H), 6.92-6.86 (m, 1H), 6.81 (d, J=8.8 Hz, 1H), 5.28 (s, 2H), 4.08 (m, 1H), 3.88 (m, 1H), 3.27-3.24 (m, 2H), 3.05-2.95 (m, 1H), 2.85 (dd, J=8.6, 3.2 Hz, 2H), 2.35 (d, J=2.0 Hz, 3H), 1.32 (d, J=1.6 Hz, 3H), 1.30 (d, J=1.6 Hz, 3H).

### Step 2. Synthesis of 1-(4-fluoro-3-methyl-phenyl)-2-isopropyl-3-[3-(2H-tetrazol-5-yl)cyclobutyl]indol-5-ol (217)

A solution of 5-benzyloxy-1-(4-fluoro-3-methyl-phenyl)-2-isopropyl-3-[3-(2H-tetrazol-5-yl)cyclobutyl]indole C108 (0.130 g, 0.258 mmol) in MeOH (5 mL) and EtOAc (5 mL) was purged with nitrogen. Pd/C (0.050 g, 0.047 mmol, wet, Degussa). The reaction mixture was evacuated and purged with hydrogen and stirred under a hydrogen atmosphere for 2 hours. The crude mixture was filtered through a pad of celite and the filtrate was concentrated in vacuo. The resulting residue was purified by silica gel chromatography (12 g ISCO column) using 0-70% EtOAc/heptanes gradient to afford 74 mg of product. 1-(4-fluoro-3-methyl-phenyl)-2-isopropyl-3-[3-(2H-tetrazol-5-yl)cyclobutyl]indol-5-ol (67%). ¹H NMR (400 MHz, DMSO-d6) δ 8.74 (s, 1H), 7.45-7.26 (m, 4H), 7.20-7.18 (m, 1H), 6.66-6.21 (m, 2H), 4.04-4.02 (m, 1H), 3.84-3.82 (m, 1H), 3.03-2.82 (m, 3H), 2.71-2.69 (m, 2H), 2.30 (d, J=1.9 Hz, 3H), 1.26 (d, J=7.2 Hz, 6H). ESI-MS m/z calc. 405.2, found 404.6 (M+1)⁺.

### Compound 218

### 1-(4-fluoro-3-methyl-phenyl)-2-tetrahydropyran-4-yl-3-[3-(2H-tetrazol-5-yl)cyclobutyl]indol-5-ol (218)

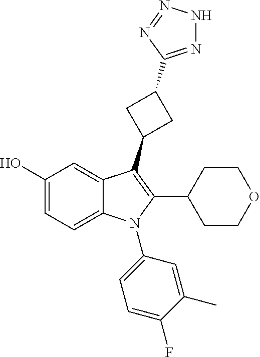 [SMILES: Cc1cc(-n2c(C3CCOCC3)c([C@H]3C[C@H](c4nn[nH]n4)C3)c3cc(O)ccc32)ccc1F]

Compound 218 was prepared from 5-(benzyloxy)-1-(4-fluoro-3-methylphenyl)-2-(tetrahydro-2H-pyran-4-yl)-1H-indole (S5) as described in the preparation of 217. Hydrogenation with Pd/C in EtOAc afforded final product. Purification by reverse phase flash chromatography (RF ISCO, C18 column, 30 g) eluting with CH₃CN/water (0-100%, 0.1% TFA) afforded the trans-isomer. ¹H NMR (400 MHz, DMSO-d6) δ 7.44-7.22 (m, 3H), 6.63-6.51 (m, 3H), 4.35-4.33 (m, 1H), 3.98 (s, 1H), 3.85-3.82 (m, 2H), 3.26-3.19 (m, 3H), 2.99-2.73 (m, 3H), 2.64 (d, J=11.3 Hz, 3H), 2.31 (s, 3H), 1.68-1.65 (m, 4H). ESI-MS m/z calc. 447.21, found 448.03 (M+1)⁺.

### Compounds 219 and 220

### Cis-3-[1-(4-fluoro-3-methyl-phenyl)-5-hydroxy-2-isopropyl-indol-3-yl]-N-methylsulfonyl-cyclobutanecarboxamide (219) and trans-3-[1-(4-fluoro-3-methyl-phenyl)-5-hydroxy-2-isopropyl-indol-3-yl]-N-methylsulfonyl-cyclobutanecarboxamide (220)

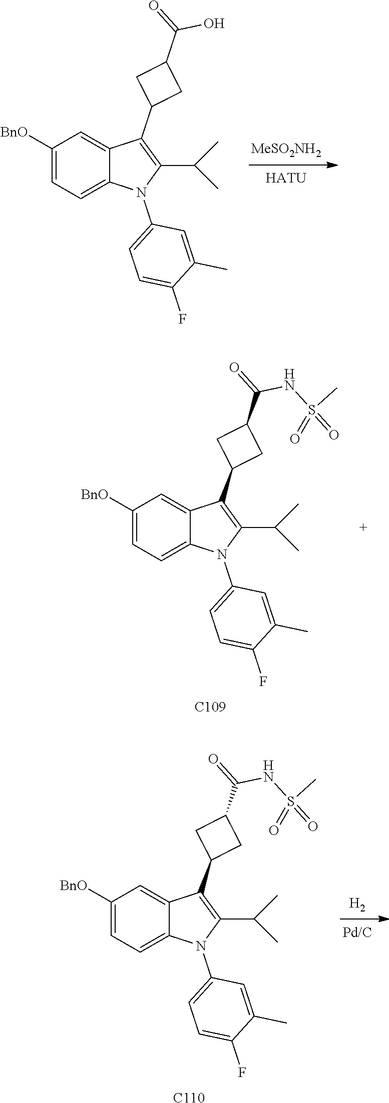

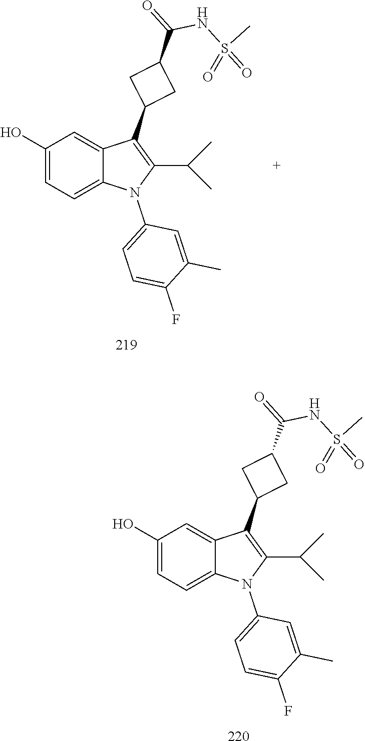 [SMILES: Cc1cc(-n2c(C(C)C)c([C@H]3C[C@@H](C(=O)NS(C)(=O)=O)C3)c3cc(O)ccc32)ccc1F ; "219" ; Cc1cc(-n2c(C(C)C)c([C@H]3C[C@H](C(=O)NS(C)(=O)=O)C3)c3cc(O)ccc32)ccc1F ; "220"]

### Step 1. Synthesis of cis-3-[5-benzyloxy-1-(4-fluoro-3-methyl-phenyl)-2-isopropyl-indol-3-yl]-N-methylsulfonyl-cyclobutanecarboxamide (C109) and trans-3-[5-benzyloxy-1-(4-fluoro-3-methyl-phenyl)-2-isopropyl-indol-3-yl]-N-methylsulfonyl-cyclobutanecarboxamide (C110)

A solution of cis/trans mixture of 3-[5-benzyloxy-1-(4-fluoro-3-methyl-phenyl)-2-isopropyl-indol-3-yl]cyclobutanecarboxylic acid (0.38 g, 0.46 mmol), methanesulfonamide (0.09 g, 0.94 mmol), HATU (0.35 g, 0.92 mmol) and diisopropylethyl amine (0.25 mL, 1.44 mmol) in DMF (5 mL) was stirred at room temperature for 3 days. The mixture was diluted into water and extracted three times with EtOAc. The organic phase was dried (MgSO₄), filtered, and concentrated in vacuo. The resulting residue was purified by silica gel chromatography (40 g ISCO column) using 0-60% EtOAc/heptanes gradient to afford products. cis isomer 3-[5-benzyloxy-1-(4-fluoro-3-methyl-phenyl)-2-isopropyl-indol-3-yl]-N-methylsulfonyl-cyclobutanecarboxamide C110 (60 mg, 24%) ESI-MS m/z calc. 548.2, found 549.0. trans isomer 3-[5-benzyloxy-1-(4-fluoro-3-methyl-phenyl)-2-isopropyl-indol-3-yl]-N-methylsulfonyl-cyclobutanecarboxamide C111 (106 mg, 41%) ESI-MS m/z calc. 548.2, found 549.7 (M+1)⁺. ¹H NMR (400 MHz, Chloroform-d) δ 8.29 (brs, 1H), 7.56-7.50 (m, 2H), 7.45-7.33 (m, 4H), 7.16-7.06 (m, 3H), 6.91-6.77 (m, 2H), 5.18 (s, 2H), 4.23-4.21 (m, 1H), 3.40 (s, 3H), 3.04-2.85 (m, 4H), 2.75-2.71 (m, 2H), 2.35 (d, J=1.9 Hz, 3H), 1.25-1.13 (m, 6H).

### Step 2. Synthesis of Cis-3-[1-(4-fluoro-3-methyl-phenyl)-5-hydroxy-2-isopropyl-indol-3-yl]-N-methylsulfonyl-cyclobutanecarboxamide (219) and trans-3-[1-(4-fluoro-3-methyl-phenyl)-5-hydroxy-2-isopropyl-indol-3-yl]-N-methylsulfonyl-cyclobutanecarboxamide (220)

A solution of 3-[5-benzyloxy-1-(4-fluoro-3-methyl-phenyl)-2-isopropyl-indol-3-yl]-N-methylsulfonyl-cyclobutanecarboxamide (0.060 g, 0.109 mmol) in MeOH (5 mL) was purged with nitrogen. Pd/C (0.030 g, 0.028 mmol, wet, Degussa) The reaction mixture was evacuated and purged with hydrogen and stirred under a hydrogen atmosphere for 2 hours. The crude mixture was filtered through a pad of celite and the filtrate was concentrated in vacuo. The resulting residue was purified by silica gel chromatography (12 g ISCO column) using 0-60% EtOAc/heptanes gradient to afford 36 mg of product. 3-[1-(4-fluoro-3-methyl-phenyl)-5-hydroxy-2-isopropyl-indol-3-yl]-N-methylsulfonyl-cyclobutanecarboxamide (71%). Trans isomer: ¹H NMR (300 MHz, Methanol-d4) δ 7.50 (d, J=2.2 Hz, 1H), 7.26-7.02 (m, 3H), 6.65-6.40 (m, 2H), 4.03-3.76 (m, 1H), 3.32 (s, 3H), 3.18-3.16 (m, 1H), 3.10-2.82 (m, 3H), 2.58-2.46 (m, 2H), 2.33 (d, J=2.0 Hz, 3H), 1.27 (d, J=7.2 Hz, 6H). ESI-MS m/z calc. 458.2, found 458.5 (M+1)⁺. Cis isomer ESI-MS m/z calc. 458.2, found 459.0 (M+1)⁺.

### Compounds 221 and 222

### Trans-5-[3-[1-(4-fluoro-3-methyl-phenyl)-5-hydroxy-2-isopropyl-indol-3-yl]cyclobutyl]-3H-1,3,4-oxadiazol-2-one (221) and cis-5-[3-[1-(4-fluoro-3-methyl-phenyl)-5-hydroxy-2-isopropyl-indol-3-yl]cyclobutyl]-3H-1,3,4-oxadiazol-2-one (222)

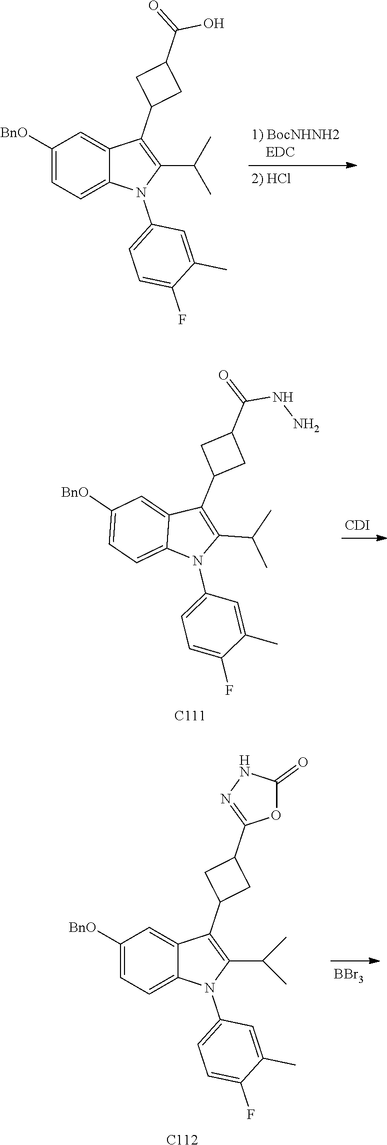

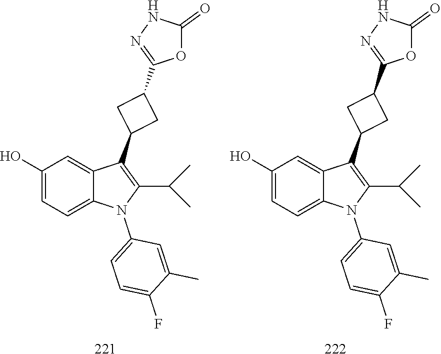 [SMILES: Cc1cc(-n2c(C(C)C)c([C@H]3C[C@H](c4n[nH]c(=O)o4)C3)c3cc(O)ccc32)ccc1F ; Cc1cc(-n2c(C(C)C)c([C@H]3C[C@@H](c4n[nH]c(=O)o4)C3)c3cc(O)ccc32)ccc1F ; "221" ; "222"]

### Step 1. Synthesis of 3-[5-benzyloxy-1-(4-fluoro-3-methyl-phenyl)-2-isopropyl-indol-3-yl]cyclobutanecarbohydrazide hydrochloride (C111)

To a solution of 3-[5-benzyloxy-1-(4-fluoro-3-methyl-phenyl)-2-isopropyl-indol-3-yl]cyclobutanecarboxylic acid (0.60 g, 1.27 mmol), EDC (0.32 g, 1.65 mmol), 1-hydroxybenzotriazole-hydrate (0.25 g, 1.65 mmol), and triethylamine (0.45 mL, 3.18 mmol) in CH₂Cl₂ (6 mL) was added tert-butyl N-aminocarbamate (0.20 g, 1.53 mmol). The reaction mixture was stirred overnight at room temperature. The mixture was diluted into water and the organic layer was separated and concentrated to dryness. The resulting residue was purified by silica gel chromatography using 0-50% EtOAc/heptanes gradient to afford 615 mg of product as a mixture of cis and trans isomers. tert-Butyl N-[[3-[5-benzyloxy-1-(4-fluoro-3-methyl-phenyl)-2-isopropyl-indol-3-yl]cyclobutanecarbonyl]-amino]carbamate (83%). ¹H NMR (400 MHz, Chloroform-d) δ 7.61-7.51 (m, 2H), 7.45-7.30 (m, 3H), 7.22-7.05 (m, 4H), 6.88-6.75 (m, 2H), 5.23 (d, J=44.4 Hz, 2H), 4.33-3.85 (m, 1H), 3.12 (dt, J=40.4, 9.2 Hz, 2H), 3.01-2.85 (m, 2H), 2.71 (t, J=10.5 Hz, 1H), 2.54 (q, J=9.3, 8.0 Hz, 1H), 2.39-2.31 (m, 3H), 1.51 (d, J=22.9 Hz, 9H), 1.30-1.23 (m, 6H). ESI-MS m/z calc. 585.3, found 586.0 (M+1)⁺.

A solution of the hydrazide product (0.60 g, 1.02 mmol) in HCl (7.0 mL of 4 M solution in dioxane, 28.0 mmol) was stirred at room temperature for 15 minutes and concentrated to dryness to afford 534 mg of product. Crude product was used without further purification in step 2. 3-[5-benzyloxy-1-(4-fluoro-3-methyl-phenyl)-2-isopropyl-indol-3-yl]cyclobutanecarbohydrazide HCl (100%). ESI-MS m/z calc. 485.2, found 486.0 (M+1)⁺.

### Step 2. Synthesis of 5-[3-[5-benzyloxy-1-(4-fluoro-3-methyl-phenyl)-2-isopropyl-indol-3-yl]cyclobutyl]-3H-1,3,4-oxadiazol-2-one (C112)

To a solution of 3-[5-benzyloxy-1-(4-fluoro-3-methyl-phenyl)-2-isopropyl-indol-3-yl]cyclobutanecarbohydrazide (Hydrochloride salt) C111 (0.500 g, 0.958 mmol) and triethylamine (0.400 mL, 2.870 mmol) in CH₂C₂ (9 mL) was added carbonyl diimidazole (0.200 g, 1.233 mmol). The reaction mixture was stirred at room temperature for 3 hours. Another 100 mg of carbonyl diimidazole was added and the reaction was stirred for 20 minutes. The reaction was then washed with water, dried over magnesium sulfate, filtered, and concentrated to dryness. The resulting residue was purified by silica gel chromatography (40 g ISCO column) using 0-65% EtOAc/heptanes gradient to afford 300 mg of product. 5-[3-[5-benzyloxy-1-(4-fluoro-3-methyl-phenyl)-2-isopropyl-indol-3-yl]cyclobutyl]-3H-1,3,4-oxadiazol-2-one (61%). ESI-MS m/z calc. 511.2, found 512.0 (M+1)⁺.

### Step 3. Synthesis of trans-5-[3-[1-(4-fluoro-3-methyl-phenyl)-5-hydroxy-2-isopropyl-indol-3-yl]cyclobutyl]-3H-1,3,4-oxadiazol-2-one (221) and cis-5-[3-[1-(4-fluoro-3-methyl-phenyl)-5-hydroxy-2-isopropyl-indol-3-yl]cyclobutyl]-3H-1,3,4-oxadiazol-2-one (222)

To a cold (0° C.) solution of 5-[3-[5-benzyloxy-1-(4-fluoro-3-methyl-phenyl)-2-isopropyl-indol-3-yl]cyclobutyl]-3H-1,3,4-oxadiazol-2-one C112 (0.300 g, 0.586 mmol) in CH₂C₂ (5 mL) was added dropwise BBr₃ (0.645 mL of 1 M solution in CH₂Cl₂, 0.645 mmol). The reaction mixture was quenched with water and the organic layer was concentrated to dryness. The crude residue was purified by reverse phase flash chromatography (RF ISCO, C18 column, 30 g) eluting with CH₃CN/water (0-100%, 0.1% TFA) to afford the product as a mixture of cis and trans products. Trans-product (221): 5-[3-[1-(4-fluoro-3-methyl-phenyl)-5-hydroxy-2-isopropyl-indol-3-yl]cyclobutyl]-3H-1,3,4-oxadiazol-2-one (35 mg, 27%). ¹H NMR (400 MHz, Chloroform-d) δ 9.36 (s, 1H), 7.42-7.33 (m, 1H), 7.16-6.99 (m, 3H), 6.74 (d, J=8.7 Hz, 1H), 6.66 (dd, J=8.7, 2.3 Hz, 1H), 4.33-4.21 (m, 1H), 3.74-3.64 (m, 1H), 3.18 (q, J=9.7 Hz, 2H), 2.93 (hept, J=7.2 Hz, 1H), 2.71 (ddd, J=13.3, 9.8, 3.4 Hz, 2H), 2.33 (d, J=2.0 Hz, 3H), 1.26-1.22 (m, 6H). ESI-MS m/z calc. 421.2, found 422.0 (M+1)⁺. Cis product (222): 5-[3-[1-(4-fluoro-3-methyl-phenyl)-5-hydroxy-2-isopropyl-indol-3-yl]cyclobutyl]-3H-1,3,4-oxadiazol-2-one (78 mg, 60%). ¹H NMR (400 MHz, Chloroform-d) δ 10.14 (s, 1H), 7.58 (dd, J=2.0, 0.9 Hz, 1H), 7.15-7.00 (m, 3H), 6.78-6.71 (m, 2H), 3.95 (tt, J=10.5, 8.3 Hz, 1H), 3.45 (tt, J=10.0, 8.0 Hz, 1H), 3.20-3.08 (m, 2H), 2.93 (p, J=7.2 Hz, 1H), 2.66-2.53 (m, 2H), 2.32 (d, J=2.0 Hz, 3H), 1.29-1.26 (m, 6H). ESI-MS m/z calc. 421.18018, found 422.0 (M+1)⁺.

### Compounds 223 and 224

### Trans-3-[3-[1-(4-fluoro-3-methyl-phenyl)-5-hydroxy-2-tetrahydropyran-4-yl-indol-3-yl]cyclobutyl]-4H-1,2,4-oxadiazol-5-one (223) and cis-3-[3-[1-(4-fluoro-3-methyl-phenyl)-5-hydroxy-2-tetrahydropyran-4-yl-indol-3-yl]cyclobutyl]-4H-1,2,4-oxadiazol-5-one (224)

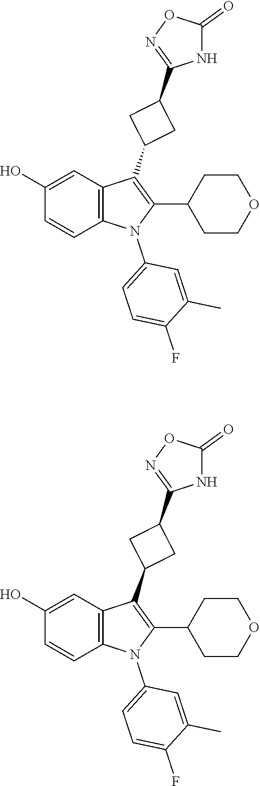 [SMILES: Cc1cc(-n2c(C3CCOCC3)c([C@H]3C[C@H](c4noc(=O)[nH]4)C3)c3cc(O)ccc32)ccc1F ; Cc1cc(-n2c(C3CCOCC3)c([C@H]3C[C@@H](c4noc(=O)[nH]4)C3)c3cc(O)ccc32)ccc1F]

Compounds 223 and 224 were prepared from 5-(benzyloxy)-1-(4-fluoro-3-methylphenyl)-2-(tetrahydro-2H-pyran-4-yl)-1H-indole (S5) as described in the preparation of 221 and 222. Benzyl deprotection with boron tribromide in CH₂Cl₂ afforded final products. Purification by SFC chiral chromatography afforded the individual isomers. trans-isomer (223): ¹H NMR (400 MHz, Chloroform-d) δ 7.32 (d, J=5.9 Hz, 1H), 7.06 (t, J=8.6 Hz, 1H), 6.96 (dd, J=22.7, 7.2 Hz, 2H), 6.72-6.58 (m, 2H), 4.07 (d, J=7.1 Hz, 2H), 3.33 (t, J=11.7 Hz, 2H), 2.68 (d, J=25.7 Hz, 3H), 2.26 (d, J=1.7 Hz, 3H), 1.20 (d, J=7.1 Hz, 8H). ESI-MS m/z calc. 463.2, found 464.0 (M+1)⁺. Cis isomer (224): ¹H NMR (400 MHz, Chloroform-d) δ 10.61 (s, 1H), 7.52-7.46 (m, 1H), 7.16 (t, J=8.7 Hz, 1H), 7.09 (dd, J=7.0, 2.5 Hz, 1H), 7.03 (ddd, J=7.8, 4.2, 2.6 Hz, 1H), 6.80-6.72 (m, 2H), 4.08 (dd, J=14.1, 6.5 Hz, 2H), 3.60-3.47 (m, 1H), 3.47-3.34 (m, 2H), 3.12 (q, J=10.6 Hz, 2H), 2.76-2.65 (m, 2H), 2.36 (d, J=1.9 Hz, 3H), 2.10 (s, 1H), 1.69 (d, J=10.5 Hz, 2H), 1.28 (s, 4H). ESI-MS m/z calc. 463.2, found 464.0 (M+1)⁺.

### Compounds 225 and 226

### Trans-1-(4-fluoro-3-methyl-phenyl)-3-[3-(hydroxymethyl)cyclobutyl]-2-isopropyl-indol-5-ol (224) and cis-1-(4-fluoro-3-methyl-phenyl)-3-[3-(hydroxymethyl)cyclobutyl]-2-isopropyl-indol-5-ol (225)

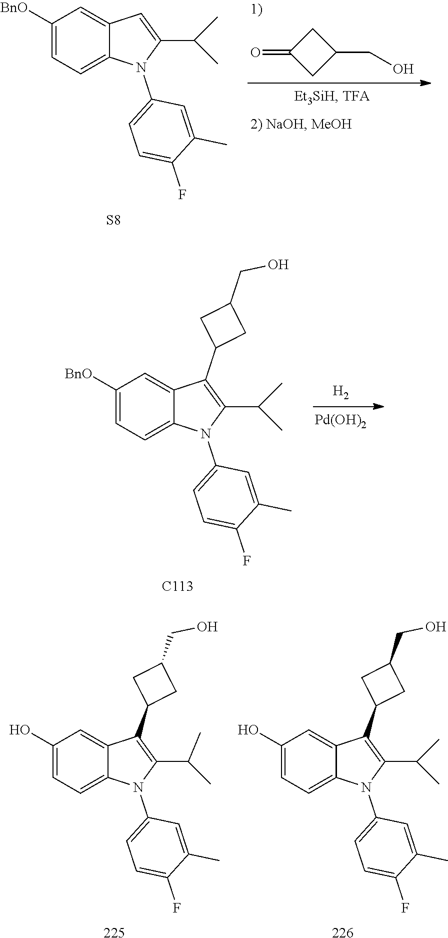

### Step 1. Synthesis of [3-[5-benzyloxy-1-(4-fluoro-3-methyl-phenyl)-2-isopropyl-indol-3-yl]cyclobutyl]methanol (C113)

To a solution of 5-benzyloxy-1-(4-fluoro-3-methyl-phenyl)-2-isopropyl-indole (0.30 g, 0.80 mmol) and 3-(hydroxymethyl)cyclobutanone (0.10 g, 0.99 mmol) in CH₂Cl₂ (3 mL) was added trifluoroacetic acid (0.25 mL, 3.25 mmol) and Et₃SiH (0.70 mL, 4.38 mmol). The mixture was heated to 45° C. and stirred overnight. The solvent was removed under reduced pressure. The resulting residue was purified by silica gel chromatography (12 g ISCO column) using 0-20% EtOAc/heptanes gradient to afford 280 mg of product. [3-[5-benzyloxy-1-(4-fluoro-3-methyl-phenyl)-2-isopropyl-indol-3-yl]cyclobutyl]methyl 2,2,2-trifluoroacetate (63%). ESI-MS m/z calc. 553.2, found 554.8 (M+1)⁺. A solution of the product ester in MeOH (10 mL) and NaOH (4.0 mL of 2 M, 8.0 mmol). The reaction mixture was stirred at room temperature for 2 hours and neutralized to pH 3 with 1N HCl solution. The aqueous phase was extracted with CH₂Cl₂, dried over Na₂SO₄, filtered and concentrated in vacuo to afford 215 mg of product. [3-[5-benzyloxy-1-(4-fluoro-3-methyl-phenyl)-2-isopropyl-indol-3-yl]cyclobutyl]methanol (58%). ESI-MS m/z calc. 457.2, found 458.5 (M+1)⁺.

### Step 2. Synthesis of Trans-1-(4-fluoro-3-methyl-phenyl)-3-[3-(hydroxymethyl)cyclobutyl]-2-isopropyl-indol-5-ol (225) and cis-1-(4-fluoro-3-methyl-phenyl)-3-[3-(hydroxymethyl)cyclobutyl]-2-isopropyl-indol-5-ol (226)

A solution of [3-[5-benzyloxy-1-(4-fluoro-3-methyl-phenyl)-2-isopropyl-indol-3-yl]cyclobutyl]methanol C113 (0.22 g, 0.47 mmol) in EtOAc (10 mL) was added Pd(OH)₂ (0.05 g, 0.36 mmol). The reaction mixture was evacuated and purged with hydrogen and stirred under a hydrogen atmosphere for 2 hours. The crude mixture was filtered through a pad of celite and the filtrate was concentrated in vacuo. The resulting residue was purified by silica gel chromatography (4 g ISCO column) using 0-30% EtOAc/CH₂Cl₂ gradient to afford 12 mg of trans product. Trans-1-(4-fluoro-3-methyl-phenyl)-3-[3-(hydroxymethyl)cyclobutyl]-2-isopropyl-indol-5-ol (6%). ¹H NMR (400 MHz, Chloroform-d) δ 7.41 (dd, J=2.4, 0.5 Hz, 1H), 7.17-7.05 (m, 3H), 6.75 (dd, J=8.7, 0.5 Hz, 1H), 6.66 (dd, J=8.7, 2.4 Hz, 1H), 4.8 (br, 1H), 4.05 (m, 1H), 3.92 (d, J=7.4 Hz, 2H), 3.05-2.68 (m, 4H), 2.35 (d, J=2.0 Hz, 3H), 2.20 (tt, J=9.5, 3.0 Hz, 2H), 1.27-1.18 (m, 6H). ESI-MS m/z calc. 367.2, found 368.0 (M+1)⁺. Cis product 1-(4-fluoro-3-methyl-phenyl)-3-[3-(hydroxymethyl)cyclobutyl]-2-isopropyl-indol-5-ol (85 mg, 45%), ¹H NMR (400 MHz, Chloroform-d) δ 7.58 (dd, J=2.3, 0.6 Hz, 1H), 7.18-7.03 (m, 3H), 6.78-6.64 (m, 2H), 6.44 (s, 1H), 3.95-3.72 (m, 3H), 2.96 (p, J=7.2 Hz, 1H), 2.76-2.53 (m, 4H), 1.41-1.08 (m, 6H). ESI-MS m/z calc. 367.2, found 368.7 (M+1)⁺.

### Compound 227

### 1-(4-fluoro-3-methyl-phenyl)-2-tetrahydropyran-4-yl-3-[3-(2H-tetrazol-5-yl)cyclobutyl]indol-5-ol (227)

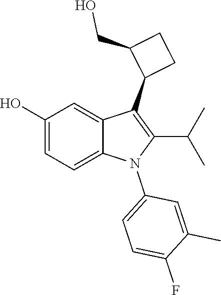 [SMILES: Cc1cc(-n2c(C(C)C)c([C@@H]3CC[C@@H]3CO)c3cc(O)ccc32)ccc1F]

Compound 227 was prepared from 5-benzyloxy-1-(4-fluoro-3-methyl-phenyl)-2-isopropyl-indole (S8) as described in the preparation of 225 and 226. Hydrogenation with Pd(OH)₂ in EtOAc afforded final product. Purification by reverse phase flash chromatography (RF ISCO, C18 column, 30 g) eluting with CH₃CN/water (0-100%, 0.1% TFA) afforded the cis-isomer. ¹H NMR (400 MHz, Chloroform-d) δ 7.50-7.34 (m, 1H), 7.14 (ddd, J=21.4, 10.5, 5.0 Hz, 3H), 6.81-6.57 (m, 2H), 4.65 (s, 1H), 4.20 (q, J=9.2 Hz, 1H), 3.96 (s, 1H), 3.72 (d, J=8.9 Hz, 2H), 3.31 (d, J=8.4 Hz, 1H), 2.98 (ddd, J=29.7, 13.0, 7.7 Hz, 2H), 2.64 (t, J=10.2 Hz, 1H), 2.55-2.12 (m, 5H), 1.95 (ddd, J=27.7, 23.8, 13.2 Hz, 1H), 1.28 (ddd, J=21.5, 12.9, 4.6 Hz, 6H). ESI-MS m/z calc. 367.2, found 368.0 (M+1)⁺.

### Assays for Detecting and Measuring AAT Modulator Properties of Compounds

A. AAT Function Assay (MSD Assay NL20-SI Cell Line)

Alpha-1 antitrypsin (AAT) is a SERpIN (serine protease inhibitor) that inactivates enzymes by binding to them covalently. This assay measured the amount of functionally active AAT in a sample in the presence of the disclosed Compounds 1-227 by determining the ability of AAT to form an irreversible complex with human neutrophil Elastase (hNE). In practice, the sample (cell supernatant, blood sample, or other) was incubated with excess hNE to allow AAT-Elastase complex to be formed with all functional AAT in the sample. This complex was then captured to a microplate coated with an anti-AAT antibody. The complex captured to the plate was detected with a labeled anti-Elastase antibody and quantitated using a set of AAT standards spanning the concentration range present in the sample. Meso Scale Discovery (MSD) plate reader, Sulfo-tag labeling, and microplates were used to provide high sensitivity and wide dynamic range.

Materials:

| Reagents/Plates | Concentration |
|---|---|
| Goat anti-human Alpha-1-Antitrypsin | 1 mL @ 1 mg/mL |
| Polyclonal Antibody | |
| Use at 5 μg/mL in phosphate buffered saline (PBS) | |
| Human Neutrophil Elastase | 100 μg lyophilized |
| Stock at 3.4 μM (0.1 mg + 1 mL PBS) | |
| Working at 1 μg/mL (34 nm) in MSD Assay buffer | |
| (1% bovine serum albumin (BSA)) | |
| Mouse anti-human Neutrophil Elastase Monoclonal | 900 μg/mL |
| Antibody | |
| Sulfo-tagged @ 12:1 using MSD Gold Sulfo-tag N- | |
| hydroxysuccinimide (NHS) ester; use at 0.45 μg/mL | |
| in MSD Assay buffer (1% BSA) | |
| M-AAT (Alpha-1-Antitrypsin) | 5 mg lyophilized |
| MSD Blocker A (BSA) | 250 mL |
| 5% solution in PBS for blocking | |
| 1% solution in PBS for assay buffer | |
| MSD Read Buffer T (4X) with Surfactant | 1 L or 250 mL |
| MSD 384 high bind plates | |
| Polypropylene for dilution 384 well plate | |
| Tissue culture treated black well 384 well plate | |

Instrument(S):
Meso Sector S600
Bravo
Washer dispenser
Multidrop Combi

Assay Protocol

Day 1 Cell Culture
1. Harvest NL20 human bronchial epithelial cells expressing human Z-AAT in OptiMEM™ containing Pen/Strep (P/S)
2. Seed at 16,000 cells/well in 30 μL (384 well plate)
3. Centrifuge plates briefly up to speed (1200 rpm) and place into 37° C. incubator overnight

Day 2: Compound Addition and Coating Plates with Capture Antibody

Compound Addition:
1. Dispense 40 μL of OptiMEM™ (P/S) with doxycycline (1:1000 stock=0.1 μM final) to each well of the compound plate using a multidrop Combi in hood
2. Remove cell plate from incubator, flip/blot and take immediately to Bravo to transfer compounds
3. Return plates to incubator overnight

Coat MSD Plates
1. Dilute capture antibody (Polyclonal Goat anti-AAT) to 5 μg/mL (1:200) in PBS (no BSA).
2. Dispense 25 μL of diluted capture antibody into all wells of MSD 384-well High Bind plate using the Multidrop equipped with a standard cassette.
3. Incubate overnight at 4° C.

Prepare Blocker A (BSA) Solutions
1. Prepare solution of 5% MSD Blocker A (BSA) following the manufacturer's instructions.
2. Further dilute the 5% MSD Blocker A in PBS to 1% (Blocker A) as needed.

Day 3: Run MSD Assay

Block Plates
1. Wash plate 1× with 50 μL Wash buffer (PBS+0.5% Tween 20), and adds 35 μL 5% Block A buffer to block non-specific binding on washer dispenser
2. Rotate plates on shaker for 1 hour at 600 rpm

Prepare M-AA T Standards
1. Dilute M-AAT stock to 1.6 μg/mL in 1% BSA Blocker A (Stock in −70° C.); then prepare 12×1:2 serial dilutions in 1% Blocker A
2. The top starting final concentration on MSD plate is 320 ng/mL. These dilutions correspond to a final concentration of 320, 160, 80, 40, 20, 10, 5, 2.5, 1.25, 0.625, 0.312, 0.156 ng/mL.

Dilution Plate
1. Add 80 μL of 1% Assay buffer to all wells except columns 1/24 (standards) with Multidrop Combi
2. Add diluted standards to columns 1 and 24
3. Centrifuge dilution plates 1200 rpm briefly

Cell Plate
1. Aspirate columns which will have the standards from the cell plates in the hood using 16-pin aspirator

Prepare Human Neutrophil Elastase (hNE)
1. Prepare 1 μg/mL Human Neutrophil Elastase by diluting in 1% Blocker A.
   a. Small 100 μg vial—add 1 mL PBS (100 μg/mL)
      i. This can then be diluted 1:100 in 1% Assay Buffer for a final 1 μg/mL concentration

MSD—add hNE (20 μL/well)
1. After the MSD plate has blocked for at least 1 hour, wash plate 1× with 50 μL Wash buffer (PBS+0.5% Tween 20) and then add 20 μL hNE to each well

Bravo—Cell Plate—Dilution Plate—MSD Plate

Using the Bravo aspirate 10 μL from the cell plate, transfer to the dilution plate (9-fold dilution)
1. Mix 25 μL 3×, then aspirate 5 μL, transfer to MSD plate (5-fold dilution)
2. Mix 10 μL 3×. Total dilution is 45 fold.
3. Shake plates at 600 rpm for 1.5 hours

Add Functional Detection hNE Antibody
1. Wash plate 1× with wash buffer
2. Add 25 μL Sulfo-tagged anti-Elastase Monoclonal Mouse anti-Elastase) diluted to 0.45 μg/mL (1:2000) in 1% Blocker A into all wells of the functional activity MSD plates using the washer/dispenser
   Note: The dilution required for sufficient signal must be determined for each new lot of labeled antibody.
3. Incubate at RT shaking at 600 rpm for 1 hour.

Final Wash and MSD Imager Read
1. Wash the plate 1×, and add 25 μL of Wash Buffer to the plate.
2. Make 2× Read buffer
3. Remove wash buffer from MSD plate
4. Transfer 35 μL 2× Read Buffer to MSD plate using Bravo and take to MSD to read immediately
Data analysis in MSD Discovery Workbench 4.0 software and EC₅₀ values were determined using Genedata. See Table 17 for data.

B. Biochemical Assay (Z-AAT Elastase Activity Assay)

This assay measured the modulation of Compounds 1-227 on Z-AAT SERpIN activity using purified Z-AAT protein and purified human neutrophil elastase (hNE). Normally, when active monomeric Z-AAT encounters a protease such as trypsin or elastase, it forms a 1:1 covalent “suicide” complex in which both the AAT and protease are irreversibly inactivated. However, compounds binding to Z-AAT can lead to a decrease in SERpIN activity. In such cases, when a protease encounters compound-bound Z-AAT, the protease cleaves and inactivates Z-AAT without itself being inactivated.

Materials

Reagents
PBS buffer (media prep)+0.01% BRIJ35 detergent (Calbiochem catalog #203728) Opti-MEM media (Fisher 11058-021)
Human neutrophil elastase (hNE, Athens Research #16-14-051200)
   3.4 μM stock (0.1 mg/mL) prepared in 50 mM Na Acetate, pH 5.5, 150 mM NaCl, stored at −80° C.
Elastase substrate V (ES V, fluorescent peptide substrate MeOSuc-Ala-Ala-Pro-Val-AMC, Calbiochem catalog #324740)
   20 mM stock in DMSO, stored at −20° C.
Purified Z-AAT protein from human plasma;
   12.9 μM (0.67 mg/mL) Z-AAT Vertex Cambridge Sample 4942, from patient
   #061-SSN, stored at −80 C

Plates
Corning 4511 (384 well black low volume)

Instruments
PerkinElmer® EnVision™

Assay Protocol

Pre-Incubation of Z-AAT with Compounds
1. 7.5 μL of Z-AAT (20 nM) was incubated with compounds 1-227 in a GCA plate for 1 hour at room temperature

Addition of hNE
1. 7.5 ul of HNE solution (3 nM in PBS+0.01% BRIJ35) added into GCA plate
2. Incubate plate for 30 minutes to allow Z-AAT/HNE suicide complex formation.

Addition of Substrate and Read Plate on PE Envision
1. 7.5 μL of substrate (300 μM solution of elastase substrate (ES V) in PBS+0.01% BRIJ35) dispensed per well into GCA plate
2. Immediately read on Envision.

C. EC50 and Z-AAT Elastase Activity Data for Compounds 1-227

The compounds of Formula (I) are useful as modulators of AAT activity. Table 17 below illustrates the EC₅₀ of the Compounds 1-227 using procedures described in Section A above. Table 17 below also provides the Z-AAT elastase activity using procedures described in Section B above. In Table 17 below, the following meanings apply: For EC₅₀ “+++” means<0.5 μM; “++” means between 0.5 μM and 2.0 μM; “+” means greater than 2.0 μM. For IC₅₀: “+++” means<2.0 μM; “++” means between 2.0 μM and 5.0 μM; “+” means greater than 5.0 μM; and “N/A” means activity not assessed. For IC₅₀, “N.D.” means activity not detected up to 30 μM.

TABLE 17
EC₅₀ and IC₅₀ data for Compounds 1-227

| Compound | NL20 Functional | Z-AAT Elastase |
|---|---|---|
| No. | EC₅₀ (μM) | Activity IC₅₀ (μM) |
| 1 | ++ | N.D. |
| 2 | + | N.D. |
| 3 | ++ | N.D. |
| 4 | + | N.D. |
| 5 | + | N.D. |
| 6 | + | N.D. |
| 7 | + | N.D. |
| 8 | ++ | N.D. |
| 9 | ++ | + |
| 10 | + | N.D. |
| 11 | +++ | N.D. |
| 12 | +++ | + |
| 13 | + | N.D. |
| 14 | ++ | N.D. |
| 15 | + | N.D. |
| 16 | + | N.D. |
| 17 | ++ | + |
| 18 | + | N.D. |
| 19 | + | N.D. |
| 20 | + | N.D. |
| 21 | + | N.D. |
| 22 | ++ | ++ |
| 23 | ++ | N.D. |
| 24 | + | N.D. |
| 25 | ++ | N.D. |
| 26 | + | N.D. |
| 27 | ++ | + |
| 28 | + | N.D. |
| 29 | ++ | N.D. |
| 30 | ++ | N.D. |
| 31 | +++ | + |
| 32 | +++ | + |
| 33 | +++ | + |
| 34 | + | N.D. |
| 35 | + | N.D. |
| 36 | + | N.D. |
| 37 | ++ | + |
| 38 | + | N.D. |
| 39 | + | N.D. |
| 40 | + | N.D. |
| 41 | ++ | N.D. |
| 42 | ++ | N.D. |
| 43 | + | N.D. |
| 44 | + | N.D. |
| 45 | + | N.D. |
| 46 | + | N.D. |
| 47 | + | N.D. |
| 48 | + | N.D. |
| 49 | + | N.D. |
| 50 | ++ | N.D. |
| 51 | ++ | N.D. |
| 52 | + | N.D. |
| 53 | + | N.D. |
| 54 | + | N.D. |
| 55 | + | N.D. |
| 56 | ++ | N.D. |
| 57 | +++ | +++ |
| 58 | ++ | ++ |
| 59 | + | + |
| 60 | +++ | + |
| 61 | ++ | + |
| 62 | ++ | N.D. |
| 63 | ++ | + |
| 64 | + | N.D. |
| 65 | + | N.D. |
| 66 | ++ | N.D. |
| 67 | + | N.D. |
| 68 | ++ | + |
| 69 | + | + |
| 70 | ++ | N.D. |
| 71 | ++ | + |
| 72 | + | N.D. |
| 73 | + | N.D. |
| 74 | + | N.D. |
| 75 | ++ | + |
| 76 | + | N.D. |
| 77 | ++ | + |
| 78 | + | + |
| 79 | ++ | + |
| 80 | ++ | +++ |
| 81 | ++ | N.D. |
| 82 | + | N.D. |
| 83 | + | N.D. |
| 84 | + | N.D. |
| 85 | + | N.D. |
| 86 | + | N.D. |
| 87 | + | N.D. |
| 88 | + | N.D. |
| 89 | ++ | ++ |
| 90 | ++ | + |
| 91 | + | N.D. |
| 92 | + | + |
| 93 | + | N.D. |
| 94 | + | N.D. |
| 95 | +++ | + |
| 96 | ++ | N.D. |
| 97 | +++ | ++ |
| 98 | + | N.D. |
| 99 | ++ | + |
| 100 | + | N.D. |
| 101 | + | N.D. |
| 102 | + | N.D. |
| 103 | + | N.D. |
| 104 | ++ | + |
| 105 | ++ | N.D. |
| 106 | + | N.D. |
| 107 | + | N.D. |
| 108 | ++ | + |
| 109 | + | N.D. |
| 110 | + | N.D. |
| 111 | + | N.D. |
| 112 | + | N.D. |
| 113 | + | N.D. |
| 114 | ++ | N.D. |
| 115 | + | N.D. |
| 116 | + | N.D. |
| 117 | + | N.D. |
| 118 | + | N.D. |
| 119 | + | N.D. |
| 120 | + | N.D. |
| 121 | + | N.D. |
| 122 | ++ | N.D. |
| 123 | + | N.D. |
| 124 | + | N.D. |
| 125 | ++ | N.D. |
| 126 | + | N.D. |
| 127 | + | N.D. |
| 128 | + | N.D. |
| 129 | ++ | + |
| 130 | ++ | + |
| 131 | + | N.D. |
| 132 | + | N.D. |
| 133 | + | N.D. |
| 134 | ++ | + |
| 135 | + | N.D. |
| 136 | ++ | N.D. |
| 137 | + | N.D. |
| 138 | + | N.D. |
| 139 | ++ | + |
| 140 | ++ | + |
| 141 | ++ | + |
| 142 | + | N.D. |
| 143 | + | N.D. |
| 144 | + | N.D. |
| 145 | ++ | + |
| 146 | ++ | N.D. |
| 147 | + | N.D. |
| 148 | +++ | +++ |
| 149 | ++ | ++ |
| 150 | ++ | + |
| 151 | + | N.D. |
| 152 | + | N.D. |
| 153 | + | N.D. |
| 154 | + | N.D. |
| 155 | + | N.D. |
| 156 | + | N.D. |
| 157 | + | N.D. |
| 158 | + | N.D. |
| 159 | + | N.D. |
| 160 | + | N.D. |
| 161 | + | N.D. |
| 162 | + | N.D. |
| 163 | +++ | N.D. |
| 164 | +++ | +++ |
| 165 | ++ | +++ |
| 166 | + | N.D. |
| 167 | +++ | +++ |
| 168 | +++ | ++ |
| 169 | + | N.D. |
| 170 | ++ | +++ |
| 171 | + | N.D. |
| 172 | ++ | ++ |
| 173 | + | N.D. |
| 174 | + | N.D. |
| 175 | ++ | N.D. |
| 176 | ++ | N.D. |
| 177 | +++ | +++ |
| 178 | +++ | +++ |
| 179 | + | N.D. |
| 180 | + | N.D. |
| 181 | + | + |
| 182 | + | N/A |
| 183 | + | N/A |
| 184 | + | + |
| 185 | +++ | N.D. |
| 186 | +++ | + |
| 187 | ++ | N.D. |
| 188 | ++ | N.D. |
| 189 | + | N.D. |
| 190 | ++ | + |
| 191 | + | N.D. |
| 192 | + | N.D. |
| 193 | ++ | N.D. |
| 194 | ++ | N.D. |
| 195 | + | N.D. |
| 196 | + | N.D. |
| 197 | ++ | N.D. |
| 198 | + | N.D. |
| 199 | + | N.D. |
| 200 | + | N.D. |
| 201 | + | N.D. |
| 202 | + | N.D. |
| 203 | +++ | N.D. |
| 204 | + | N.D. |
| 205 | + | N.D. |
| 206 | + | N.D. |
| 207 | + | N.D. |
| 208 | + | N.D. |
| 209 | + | N.D. |
| 210 | + | N.D. |
| 211 | + | N.D. |
| 212 | + | N.D. |
| 213 | + | + |
| 214 | + | N.D. |
| 215 | + | + |
| 216 | + | + |
| 217 | ++ | + |
| 218 | + | N.D. |
| 219 | + | + |
| 220 | + | + |
| 221 | + | N/A |
| 222 | + | N/A |
| 223 | ++ | N.D. |
| 224 | + | N.D. |
| 225 | + | N.D. |
| 226 | + | N.D. |
| 227 | + | N.D. |

### Other Embodiments

This description provides merely exemplary embodiments of the disclosed subject matter. One skilled in the art will readily recognize from the disclosure and accompanying claims, that various changes, modifications and variations can be made therein without departing from the spirit and scope of the subject matter as defined in the following claims.

What is claimed is:

1. A compound represented by the following structural formula: 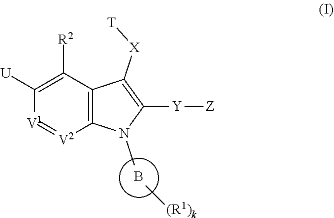
a tautomer thereof, a deuterated derivative of the compound or tautomer, or a pharmaceutically acceptable salt of any of the foregoing, wherein:
V¹ and V² are each independently N or —CR²;
U is —OH or —NH₂;
X is absent or a bond, —(CR^{a}R^{a})ₚ—, or —R^{a′}C═CR^{a′}—;
Y is absent or a bond, —(CR^{b}R^{b})_{q}—, or —R^{b′}C═CR^{b′}—;
T is —CR^{c}R^{c}COOH, —CR^{c}═CR^{c}COOH, —CN, or 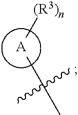
R^{a} and R^{b}, for each occurrence, are each independently hydrogen, halogen, —OH, benzyl, C₁-C₆ alkyl, C₂-C₆ alkenyl, C₁-C₆ haloalkyl, C₁-C₆ alkoxy, or C₁-C₆ haloalkoxy;
R^{a′} and R^{b′}, for each occurrence, are each independently hydrogen, C₁-C₆ alkyl, C₂-C₆ alkenyl, C₁-C₆ haloalkyl, C₁-C₆ alkoxy, or C₁-C₆ haloalkoxy;
R^{c}, for each occurrence, are independently hydrogen, halogen, —OH, benzyl, C₁-C₆ alkyl, C₂-C₆ alkenyl, C₁-C₆ haloalkyl, C₁-C₆ alkoxy, or C₁-C₆ haloalkoxy;
Ring A is C₃-C₁₂ cycloalkyl, 3 to 12-membered heterocyclyl, C₆ or C₁₀ aryl, or 5 to 10-membered heteroaryl;
Ring B is C₄-C₁₂ cycloalkyl, C₆ or C₁₀ aryl, benzyl, or 5 to 10-membered heteroaryl;
Z is —CN, 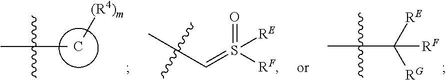
wherein:
when T is not —CN, Ring C is C₃-C₁₂ cycloalkyl, C₆ or C₁₀ aryl, 3 to 12-membered heterocyclyl, or 5 to 10-membered heteroaryl;
when T is —CN, Ring C is C₃-C₁₂ cycloalkyl or 3 to 12-membered heterocyclyl;
R^{E}, R^{F}, and R^{G} are each independently hydrogen, halogen, cyano, C₁-C₆ alkyl, C₂-C₆ alkenyl, C₁-C₆ alkoxy, C₁-C₆ haloalkyl, C₁-C₆ haloalkoxy, —C(═O)R^{s}, —C(═O)OR^{s}, —C(═O)NR^{p}R^{q}, —CR^{p}(═N)OR^{s}, —NR^{p}R^{q}, —NR^{p}C(═O)R^{s}, —NR^{p}C(═O)OR^{s}, —NR^{p}C(═O)NR^{q}R^{r}, —OR^{s}, —OC(═O)R^{s}, or —OC(═O)NR^{p}R^{q}; wherein:
the C₁-C₆ alkyl or the C₂-C₆ alkenyl of any one of R^{E}, R^{F}, and R^{G} is optionally substituted with 1 to 3 groups selected from cyano, —C(═O)R^{s}, —C(═O)OR^{s}, —C(═O)NR^{p}R^{q}, —NR^{p}C(═O)R^{s}, —NR^{p}C(═O)OR^{s}, —NR^{p}C(═O)NR^{q}R^{r}, —NR^{p}S(═O)ᵣR^{s}, —OR^{s}, —OC(═O)R^{s}, —OC(═O)OR^{s}, —OC(═O)NR^{p}R^{q}, —S(═O)ᵣR^{s}, and —S(═O)ᵣNR^{p}R^{q}; wherein:
R^{p}, R^{q}, and R^{r}, for each occurrence, are each independently hydrogen, C₁-C₄ alkyl, C₃-C₆ cycloalkyl, or 3 to 6-membered heterocyclyl; wherein:
the C₁-C₄ alkyl of any one of R^{p}, R^{q}, and R^{r} is optionally substituted with 1 to 3 groups selected from halogen, cyano, —OH, C₁-C₃ alkoxy, —C(═O)NH(C₁-C₂ alkyl), and —C(═O)N(C₁-C₂ alkyl)₂; and
the C₃-C₆ cycloalkyl or the 3 to 6-membered heterocyclyl of any one of R^{p}, R^{q}, and R^{r} is optionally substituted with 1 to 3 groups selected from halogen, cyano, —OH, C₁-C₃ alkyl, C₁-C₃ alkoxy, C₁-C₃ haloalkyl, C₁-C₃ haloalkoxy, —C(═O)O(C₁-C₂ alkyl), —C(═O)NH(C₁-C₂ alkyl), and —C(═O)N(C₁-C₂ alkyl)₂;
R^{s}, for each occurrence, is independently hydrogen, C₁-C₄ alkyl, C₃-C₆ cycloalkyl, phenyl, or 5 or 6-membered heteroaryl; wherein:
the C₁-C₄ alkyl of R^{s} is optionally substituted with 1 to 3 groups selected from halogen, cyano, —OH, —NH₂, —NH(C₁-C₂ alkyl), —N(C₁-C₂ alkyl)₂, C₁-C₃ alkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
the C₃-C₆ cycloalkyl, the phenyl. or the 5 or 6-membered heteroaryl of R^{s} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
R¹ is halogen, cyano, C₁-C₃ alkyl, C₁-C₃ haloalkyl, C₁-C₃ alkoxy, C₁-C₃ haloalkoxy, or —O—(C₃-C₆ cycloalkyl);
R², for each occurrence, is independently hydrogen, halogen, cyano, C₁-C₆ alkyl, C₂-C₆ alkenyl, C₁-C₆ alkoxy, C₁-C₆ haloalkyl, C₁-C₆ haloalkoxy, C₃-C₆ cycloalkyl, —NR^{h}Rⁱ, phenyl, or 5 or 6-membered heteroaryl; wherein:
the C₁-C₆ alkyl, the C₂-C₆ alkenyl or the C₃-C₆ cycloalkyl of R² is optionally substituted with 1 to 3 groups selected from cyano, —C(═O)R^{k}, —C(═O)OR^{k}, —C(═O)NR^{h}Rⁱ, —NR^{h}Rⁱ, —NR^{h}C(═O)R^{k}, —NR^{h}C(═O)OR^{k}, —NR^{h}C(═O)NRⁱR^{j}, —NR^{h}S(═O)ₛR^{h}, —OR^{k}, —OC(═O)R^{h}, —OC(═O)OR^{h}, —OC(═O)NR^{h}Rⁱ, —S(═O)ₛR^{k}, and S(═O)ₛNR^{h}Rⁱ; wherein:
R^{h}, Rⁱ, and R^{j}, for each occurrence, are each independently hydrogen, C₁-C₄ alkyl, or C₃-C₆ cycloalkyl; wherein:
the C₁-C₄ alkyl of any one of R^{h}, Rⁱ, and R^{j} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
the C₃-C₆ cycloalkyl of any one of R^{h}, Rⁱ, and Rⁱ is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
R^{k}, for each occurrence, is independently hydrogen, C₁-C₄ alkyl, C₃-C₆ cycloalkyl, phenyl, or 5 or 6-membered heteroaryl; wherein:
—OR^{k} cannot be —OH;
the C₁-C₄ alkyl of R^{k} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
the C₃-C₆ cycloalkyl, the phenyl, or the 5 or 6-membered heteroaryl of R^{k} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
R³ and R⁴, for each occurrence, are each independently halogen, cyano, ═O, C₁-C₆ alkyl, C₂-C₆ alkenyl, C₁-C₆ alkoxy, C₁-C₆ haloalkyl, C₁-C₆ haloalkoxy, C₃-C₆ cycloalkyl, —C(═O)R^{y}, —C(═O)OR^{y}, —C(═O)NR^{v}R^{w}, —C(═O)NR^{v}OR^{y}, -(═O)NR^{v}S(═O)ₜR^{y}, —NR^{v}R^{w}, —NR^{v}C(═O)R^{y}, —NR^{v}C(═O)OR^{y}, —NR C(═O)NR^{w}R^{x}, —NR^{v}S(═O)ₜR^{y}, —OR^{y}, —OC(═O)R^{y}, —OC(═O)OR^{y}, —OC(═O)NR^{v}R^{w}, —S(═O)ₜR^{y}, —S(═O)ₜNR^{v}R^{w}, —S(═O)ₜNR^{v}C(═O)R^{y}, —P(═O)R^{z}R^{z}, phenyl, or 5 or 6-membered heteroaryl; wherein:
the C₁-C₆ alkyl, the C₂-C₆ alkenyl, the C₃-C₆ cycloalkyl, or the 5 or 6-membered heteroaryl of any one of R³ and R⁴ is optionally substituted with 1 to 3 groups selected from cyano, —C(═O)R^{y}, —C(═O)OR^{y}, —C(═O)NR^{v}R^{w}, —NR^{v}R^{W}, —NR^{v}C(═O)R^{y}, —NR^{v}C(═O)OR^{y}, —NR^{v}C(═O)NR^{w}R^{x}, —NR^{v}S(═O)ᵣR^{y}, —OR^{y}, —OC(═O)R^{y}, —OC(═O)OR^{y}, —OC(═O)NR^{v}R^{w}, —S(═O)ₜR^{y}, and —S(═O)ₜNR^{v}R^{w}; wherein:
R^{v}, R^{w}, and R^{x}, for each occurrence, are each independently hydrogen, C₁-C₄ alkyl, C₃-C₆ cycloalkyl, 5 or 6-membered heterocyclyl, or 5 or 6-membered heteroaryl; wherein:
the C₁-C₄ alkyl of any one of R^{v}, R^{w}, and R^{x} is optionally substituted with 1 to 3 groups selected from halogen, cyano —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
the C₃-C₆ cycloalkyl, the 5 or 6-membered heterocyclyl, or the 5 or 6-membered heteroaryl of any one of R^{v}, R^{w}, and R^{x} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
R^{y}, for each occurrence, is independently hydrogen, C₁-C₄ alkyl, C₃-C₆ cycloalkyl, phenyl, a 5 or 6-membered heterocyclyl, or a 5 or 6-membered heteroaryl; wherein
the C₁-C₄ alkyl of R^{y} is optionally substituted with 1 to 3 groups selected from halogen, cyano, —OH, —NH₂, —NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂; and
the C₃-C₆ cycloalkyl, the phenyl, the 5 or 6-membered heterocyclyl, or the 5 or 6-membered heteroaryl of R^{y} is optionally substituted with 1 to 3 groups selected from halogen, cyano, —OH, —NH₂, NH(C₁-C₂ alkyl), —N(C₁-C₂ alkyl)₂, C₁-C₃ alkyl, C₁-C₃ alkoxy, C₁-C₃ haloalkyl, C₁-C₃ haloalkoxy, —C(═O)OH, —C(═O)O(C₁-C₂ alkyl), —C(═O)NH₂, —C(═O)NH(C₁-C₂ alkyl), and —C(═O)N(C₁-C₂ alkyl)₂;
R^{z}, for each occurrence, is independently C₁-C₂ alkyl, —OH, or —O(C₁-C₂ alkyl);
k, n, and o are each independently an integer selected from 0, 1, 2, and 3; and
p, q, r, s, and t are each independently an integer selected from 1 and 2.

2. The compound, tautomer, deuterated derivative, or pharmaceutically acceptable salt according claim 1, represented by Formula (IIa): 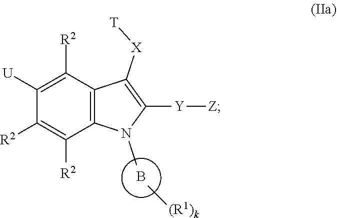
wherein:
Y is absent or a bond, —CR^{b}R^{b}—, or —R^{b′}C═CR^{b′}—;
R^{b}, for each occurrence, is independently hydrogen or C₁-C₂ alkyl;
Ring B is optionally substituted with Rⁱ and Ring B is C₄-C₆ cycloalkyl, phenyl, or 5 or 6-membered heteroaryl;
and wherein all other variables not specifically defined herein are as defined in claim 1.

3. The compound, tautomer, deuterated derivative, or pharmaceutically acceptable salt, according to claim 1 represented by Formulae (IIb) or (IIc): 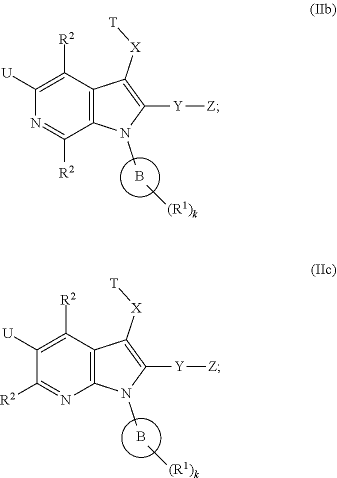
wherein:
Y is absent or a bond, —CR^{b}R^{b}—, or —R^{b′}C═CR^{b′}—;
R^{b}, for each occurrence, is independently hydrogen or C₁-C₂ alkyl;
Ring B is optionally substituted with R¹ and Ring B is C₄-C₆ cycloalkyl, phenyl, or 5 or 6-membered heteroaryl;
and wherein all other variables not specifically defined herein are as defined in claim 1.

4. The compound, tautomer, deuterated derivative, or pharmaceutically acceptable salt according to any one of claims 1 to 3, wherein Y is absent or a bond, —CH₂—, or —HC═CH—; and wherein all other variables not specifically defined herein are as defined in any one of the preceding claims.

5. The compound, tautomer, deuterated derivative, or pharmaceutically acceptable salt according to any one of claims 1 to 4, represented by Formula (III): 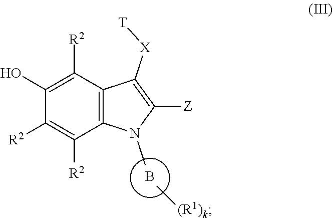
wherein:
X is absent or a bond, or —(CR^{a}R^{a})ₚ—;
R^{a}, for each occurrence, is each independently hydrogen or C₁-C₂ alkyl;
R^{c}, for each occurrence, is independently hydrogen, F, —OH, benzyl, C₁-C₂ alkyl, or C₁-C₂ alkoxy;
Ring B is optionally substituted with R¹ and Ring B is cyclobutyl, phenyl, pyridinyl, or pyrimidinyl;
and wherein all other variables not specifically defined herein are as defined in any one of the preceding claims.

6. The compound, tautomer, deuterated derivative, or pharmaceutically acceptable salt according to any one of claims 1 to 5, wherein:
X is absent or a bond, —CH₂—, —CHCH₃—, —CH₂CH₂—, or —CHCH₃CH₂—;
Ring B is optionally substituted with Rⁱ and Ring B is cyclobutyl, phenyl, pyridine-4-yl, or pyrimidin-4-yl;
and wherein all other variables not specifically defined herein are as defined in any one of the preceding claims.

7. The compound, tautomer, deuterated derivative, or pharmaceutically acceptable salt according to any one of claims 1 to 6, represented by Formula (IV): 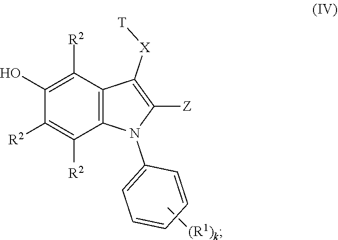
wherein:
T is —CH₂COOH, —CHCH₃COOH, —CHC₂H₅COOH, —C(CH₃)₂COOH, —CF₂COOH, —CH═CHCOOH, —C(CH₃)(OH)COOH, —C(CH₃)(OCH₃)COOH, cyano, —CH(benzyl)COOH, or Ring A optionally substituted with R³;
when Z is Ring C, Ring C is optionally substituted with R⁴ and Ring C is C₃-C₆ cycloalkyl, 4 to 8-membered heterocyclyl, phenyl, or 5 or 6-membered heteroaryl; and
R¹ is halogen, C₁-C₂ alkyl, or C₁-C₂ haloalkyl; and
k is an integer selected from 0, 1 and 2;
and wherein all other variables not specifically defined herein are as defined in any one of the preceding claims.

8. The compound, tautomer, deuterated derivative, or pharmaceutically acceptable salt according to any one of claims 1 to 7, wherein R¹ is F, Cl, or —CH₃; and wherein all other variables not specifically defined herein are as defined in any one of preceding claims.

9. The compound, tautomer, deuterated derivative, or pharmaceutically acceptable salt according to any one of claims 1 to 8, wherein when T is Ring A, Ring A is optionally substituted with R³, and Ring A is C₃-C₇ cycloalkyl, 4 to 6-membered heterocyclyl, phenyl, or 5 or 6-membered heteroaryl; and
wherein all other variables not specifically defined herein are as defined in any one of preceding claims.

10. The compound, tautomer, deuterated derivative, or pharmaceutically acceptable salt according to any one of claims 1 to 9, wherein when T is Ring A, Ring A is optionally substituted with R³, and Ring A is C₃-C₇ cycloalkyl, 4 to 6-membered heterocyclyl, phenyl, or 5 or 6-membered heteroaryl containing one or two nitrogen atoms; and wherein all other variables not specifically defined herein are as defined in any one of preceding claims.

11. The compound, tautomer, deuterated derivative, or pharmaceutically acceptable salt according to any one of claims 1 to 10, wherein when T is Ring A, Ring A is optionally substituted with R³, and Ring A is selected from 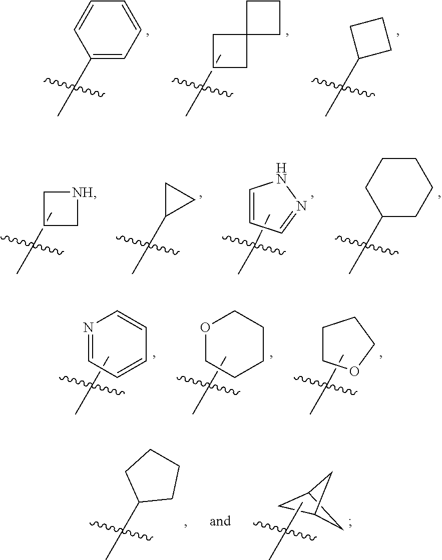
and wherein all other variables not specifically defined herein are as defined in any one of preceding claims.

12. The compound, tautomer, deuterated derivative, or pharmaceutically acceptable salt according to any one of claims 1 to 11, wherein when T is Ring A, Ring A is optionally substituted with R³, and Ring A is selected from 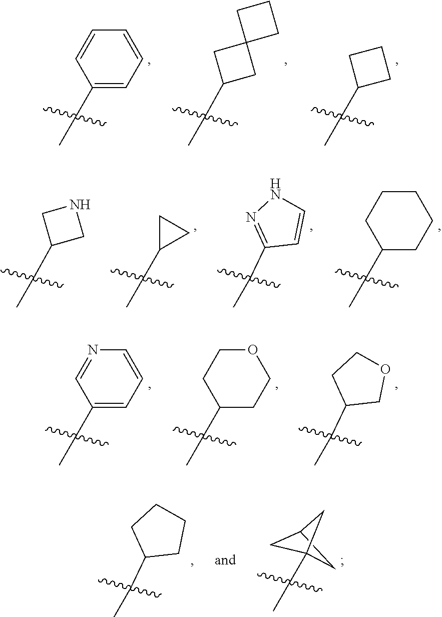
and wherein all other variables not specifically defined herein are as defined in any one of preceding claims.

13. The compound, tautomer, deuterated derivative, or pharmaceutically acceptable salt according to any one of claims 1 to 12, wherein when Z is Ring C, Ring C is optionally substituted with R⁴, and Ring C is C₃-C₄ cycloalkyl or 4 to 6-membered heterocyclyl; and wherein all other variables not specifically defined herein are as defined in any one of the preceding claims.

14. The compound, tautomer, deuterated derivative, or pharmaceutically acceptable salt according to any one of claims 1 to 13, wherein when Z is Ring C, Ring C is optionally substituted with R⁴, and Ring C is 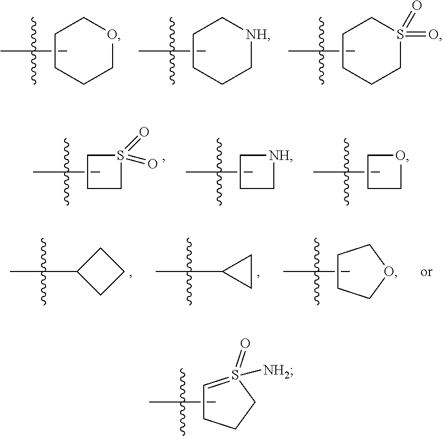
and wherein all other variables not specifically defined herein are as defined in any one of the preceding claims.

15. The compound, tautomer, deuterated derivative, or pharmaceutically acceptable salt according to any one of claims 1 to 14, wherein when Z is Ring C, Ring C is optionally substituted with R⁴, and Ring C is 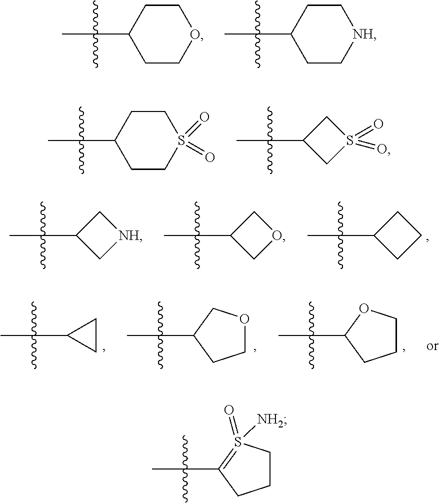
and wherein all other variables not specifically defined herein are as defined in any one of the preceding claims.

16. The compound, tautomer, deuterated derivative, or pharmaceutically acceptable salt according to any one of claims 1 to 12, wherein when Z is 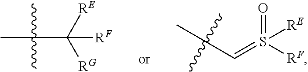
R^{E}, R^{F}, and R^{G} are each independently hydrogen, halogen, cyano, C₁-C₆ alkyl, C₁-C₆ alkoxy, C₁-C₆ haloalkyl, —C(═O)OR^{s}, —C(═O)NR^{p}R^{q}, —CR^{p}(═N)OR^{s}, —NR^{p}R^{q}, or —OR^{s}; wherein:
the C₁-C₆ alkyl of any one of R^{E}, R^{F}, and R^{G} is optionally substituted with 1 to 3 groups selected from cyano and —OR^{s}; wherein:
R^{p} and R^{a}, for each occurrence, are each independently hydrogen or C₁-C₄ alkyl; and
R^{s}, for each occurrence, is independently hydrogen or C₁-C₄ alkyl;
and wherein all other variables not specifically defined herein are as defined in any one of preceding claims.

17. The compound, tautomer, deuterated derivative, or pharmaceutically acceptable salt according to any one of claims 1 to 12 and 16, wherein when Z is 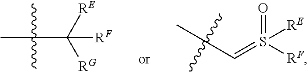
R^{E}, R^{F}, and R^{G} are each independently hydrogen, halogen, C₁-C₂ alkyl, —NR^{p}R^{q}, or —OR^{s}; wherein:
the C₁-C₂ alkyl of any one of R^{E}, R^{F}, and R^{G} is optionally substituted with 1 to 3 groups selected from cyano, —OH, and —OCH₃; wherein:
R^{p} and R^{q}, for each occurrence, are each independently hydrogen or C₁-C₂ alkyl; and
R^{s}, for each occurrence, is independently hydrogen or C₁-C₂ alkyl;
and wherein all other variables not specifically defined herein are as defined in any one of preceding claims.

18. The compound, tautomer, deuterated derivative, or pharmaceutically acceptable salt according to any one of claims 1 to 12, 16, and 17, wherein:
when Z is 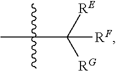
R^{E}, R^{F}, and R^{G} are each independently hydrogen, F, —CH₂CN, —OH, —OCH₃, —CH₃, —C₂H₅, or —CH₂OCH₃; and
when Z is 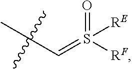
R^{E} and R^{F} are each independently —CH₃ or —NH₂;
and wherein all other variables not specifically defined herein are as defined in any one of preceding claims.

19. The compound, tautomer, deuterated derivative, or pharmaceutically acceptable salt according to any one of claims 1 to 18, represented by Formulae (Va), (Vb), or (Vc): 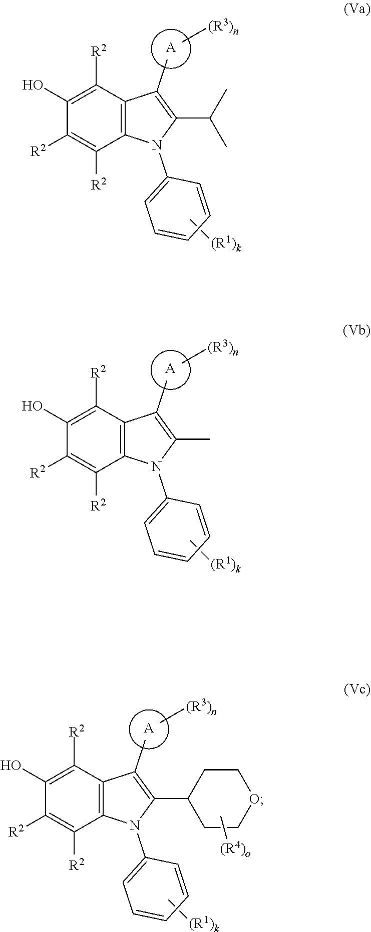
wherein all other variables not specifically defined herein are as defined in any one of preceding claims.

20. The compound, tautomer, deuterated derivative, or pharmaceutically acceptable salt according to any one of claims 1 to 19, represented by Formulae (VIa), (VIb), or (VIc): 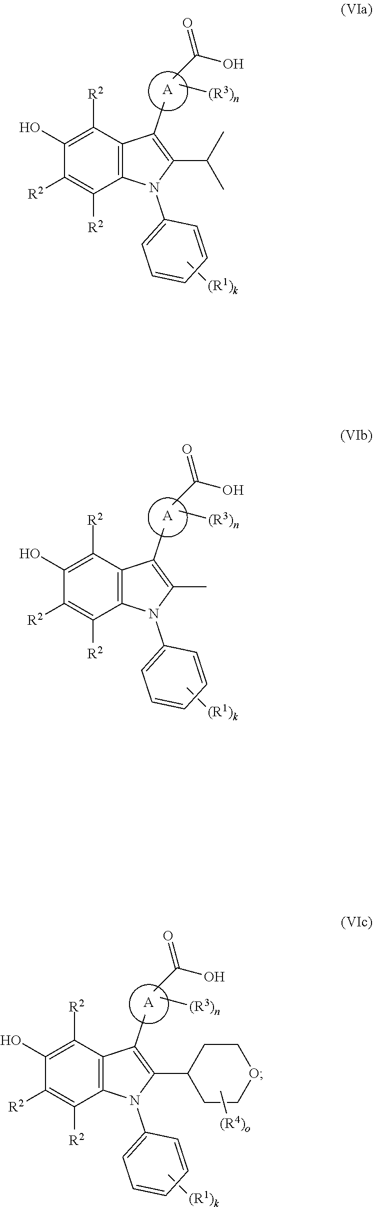
wherein n is an integer selected from 0, 1, and 2; and wherein all other variables not specifically defined herein are as defined in any one of preceding claims.

21. The compound, tautomer, deuterated derivative, or pharmaceutically acceptable salt according to any one of claims 1 to 20, represented by Formulae (VIIa), (VIIb), (VIIc), (VIId), or (VIIe): 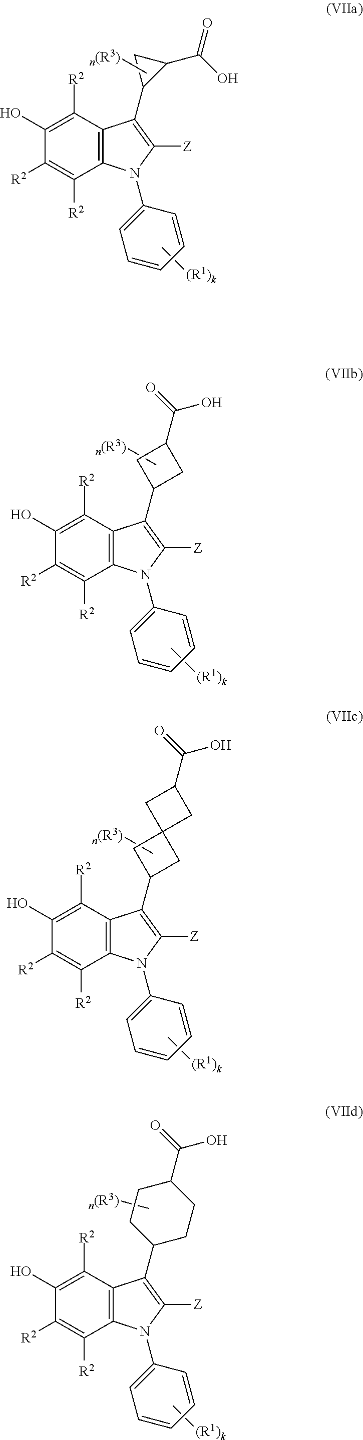 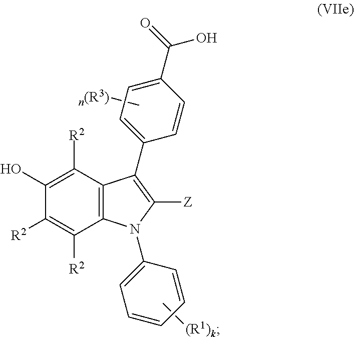
wherein n is an integer selected from 0, 1, and 2; and wherein all other variables not specifically defined herein are as defined in any one of preceding claims.

22. The compound, tautomer, deuterated derivative, or pharmaceutically acceptable salt according to any one of claims 1 to 21, wherein R², for each occurrence, is independently hydrogen, halogen, cyano, C₁-C₄ alkyl, C₁-C₄ alkoxy, C₁-C₄ haloalkyl, —NR^{h}Rⁱ, or cyclopropyl; wherein R^{h} and Rⁱ, for each occurrence, is independently hydrogen or C₁-C₄ alkyl; and wherein all other variables not specifically defined herein are as defined in any one of preceding claims.

23. The compound, tautomer, deuterated derivative, or pharmaceutically acceptable salt according to any one of claims 1 to 22, wherein R², for each occurrence, is independently hydrogen, F, Cl, —CH₃, —NH₂, or cyclopropyl; and wherein all other variables not specifically defined herein are as defined in any one of preceding claims.

24. The compound, tautomer, deuterated derivative, or pharmaceutically acceptable salt according to any one of claims 1 to 23, wherein R³, for each occurrence, is independently halogen, cyano, ═O, C₁-C₆ alkyl, C₁-C₆ alkoxy, C₁-C₆ haloalkyl, —C(═O)OR^{y}, —C(═O)NR^{v}S(═O)₂R^{y}, —S(═O)₂NR^{v}R^{w}, —S(═O)₂NR^{v}C(═O)R^{w}, —P(═O)R^{z}R^{z}, or 5 or 6-membered heteroaryl; wherein:
the C₁-C₆ alkyl or the 5-membered heteroaryl of R³ is optionally substituted with 1 to 3 groups selected from cyano, —C(═O)OR^{y}, —OR^{y}, and —NR^{v}R^{w}; wherein:
R^{v} and R^{w}, for each occurrence, are each independently hydrogen or C₁-C₄ alkyl; and
R^{y}, for each occurrence, is independently hydrogen or C₁-C₄ alkyl;
and wherein all other variables not specifically defined herein are as defined in any one of preceding claims.

25. The compound, tautomer, deuterated derivative, or pharmaceutically acceptable salt according to any one of claims 1 to 24, wherein R³, for each occurrence, is independently halogen, cyano, ═O, C₁-C₄ alkyl, C₁-C₄ alkoxy, C₁-C₄ haloalkyl, —C(═O)OR^{y}, —C(═O)NR^{v}S(═O)₂R^{y}, —S(═O)₂NR^{v}R^{w}, —S(═O)₂NR^{v}C(═O)R^{y}, or 5-membered heteroaryl; wherein:
the C₁-C₄ alkyl or the 5-membered heteroaryl of R³ is optionally substituted with 1 to 3 groups selected from cyano, —C(═O)OR^{y}, —OR^{y}, and —NR^{v}R^{w}; wherein:
R^{v} and R^{w}, for each occurrence, are each independently hydrogen or C₁-C₂ alkyl; and
R^{y}, for each occurrence, is independently hydrogen or C₁-C₂ alkyl;
and wherein all other variables not specifically defined herein are as defined in any one of preceding claims.

26. The compound, tautomer, deuterated derivative, or pharmaceutically acceptable salt according to any one of claims 1 to 25, wherein R³, for each occurrence, is independently halogen, cyano, ═O, C₁-C₂ alkyl, C₁-C₂ alkoxy, C₁-C₂ haloalkyl, —C(═O)OR^{y}, —C(═O)NR'S(═O)₂R^{y}, —S(═O)₂NR^{v}R^{w}, —S(═O)₂NR^{v}C(═O)R^{y}, tetrazolyl, or oxadiazolyl; wherein:
the C₁-C₂ alkyl or the oxadiazolyl of R³ is optionally substituted with 1 to 3 groups selected from cyano, —COOH, and —OH; wherein:
R^{v} and R^{w}, for each occurrence, are each independently hydrogen or —CH₃; and
R^{y}, for each occurrence, is independently hydrogen or —CH₃;
and wherein all other variables not specifically defined herein are as defined in any one of preceding claims.

27. The compound, tautomer, deuterated derivative, or pharmaceutically acceptable salt according to any one of claims 1 to 26, wherein R³, for each occurrence, is independently F, cyano, ═O, —CH₃, —CH₂F, —CHF₂, —CF₃, —CH₂OH, —CH₂OCH₃, —OCH₃, —COOH, —CH₂COOH, —C(═O)NHS(═O)₂CH₃, —S(═O)₂NHCH₃, —S(═O)₂NHC(═O)CH₃, tetrazol-5-yl, 1,2,4-oxadiazol-5(4H)-onyl, or 1,3,4-oxadiazol-2(3H)-onyl; and wherein all other variables not specifically defined herein are as defined in any one of preceding claims.

28. The compound, tautomer, deuterated derivative, or pharmaceutically acceptable salt according to any one of claims 1 to 27, wherein R⁴, for each occurrence, is independently halogen, cyano, C₁-C₆ alkyl, C₁-C₆ haloalkyl, —C(═O)R^{y}, —C(═O)OR^{y}, C(═O)NR^{v}R^{w}, —NR^{v}R^{w}, —OR^{y}, or —P(═O)R^{z}R^{z}; wherein:
R^{v} and R^{w}, for each occurrence, are each independently hydrogen or C₁-C₄ alkyl; and
R^{y}, for each occurrence, is independently hydrogen or C₁-C₄ alkyl;
and wherein all other variables not specifically defined herein are as defined in any one of preceding claims.

29. The compound, tautomer, deuterated derivative, or pharmaceutically acceptable salt according to any one of claims 1 to 28, wherein R⁴, for each occurrence, is independently halogen, cyano, C₁-C₄ alkyl, C₁-C₄ haloalkyl, —C(═O)R^{y}, —C(═O)OR^{y}, C(═O)NR^{v}R^{w}, —NR^{v}R^{w}, or —OR^{y}; wherein:
R^{v} and R^{w}, for each occurrence, are each independently hydrogen or C₁-C₂ alkyl; and
R^{y}, for each occurrence, is independently hydrogen or C₁-C₄ alkyl;
and wherein all other variables not specifically defined herein are as defined in any one of preceding claims.

30. The compound, tautomer, deuterated derivative, or pharmaceutically acceptable salt according to any one of claims 1 to 29, wherein R⁴, for each occurrence, is independently halogen, cyano, C₁-C₂ alkyl, C₁-C₂ haloalkyl, —C(═O)OR^{y}, or —OR^{y}; wherein:
R^{y}, for each occurrence, is independently hydrogen or C₁-C₄ alkyl;
and wherein all other variables not specifically defined herein are as defined in any one of preceding claims.

31. The compound, tautomer, deuterated derivative, or pharmaceutically acceptable salt according to any one of claims 1 to 30, wherein R⁴, for each occurrence, is —C(═O)OC(CH₃)₃; and wherein all other variables not specifically defined herein are as defined in any one of preceding claims.

32. The compound, tautomer, deuterated derivative, or pharmaceutically acceptable salt according to any one of claims 1 to 31, wherein m is 0; and wherein all other variables not specifically defined herein are as defined in any one of preceding claims.

33. A compound selected from: 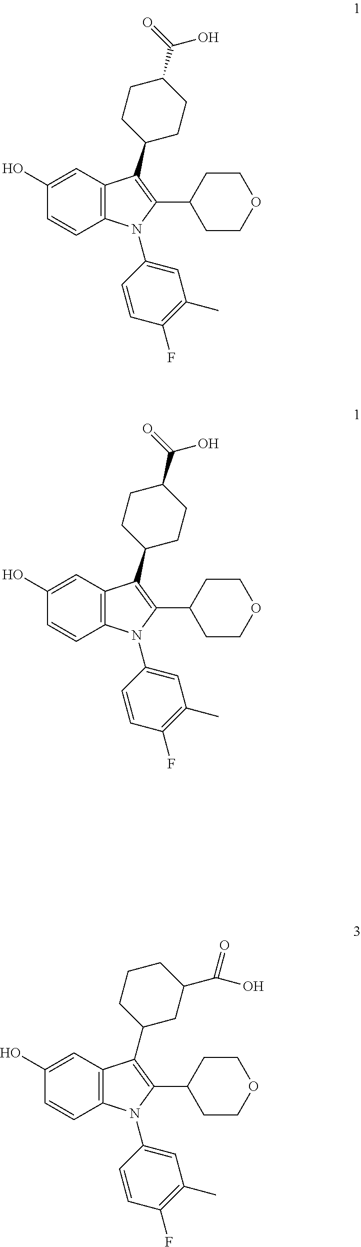 [SMILES: Cc1cc(-n2c(C3CCOCC3)c([C@H]3CC[C@H](C(=O)O)CC3)c3cc(O)ccc32)ccc1F ; Cc1cc(-n2c(C3CCOCC3)c([C@H]3CC[C@@H](C(=O)O)CC3)c3cc(O)ccc32)ccc1F ; Cc1cc(-n2c(C3CCOCC3)c(C3CCCC(C(=O)O)C3)c3cc(O)ccc32)ccc1F] 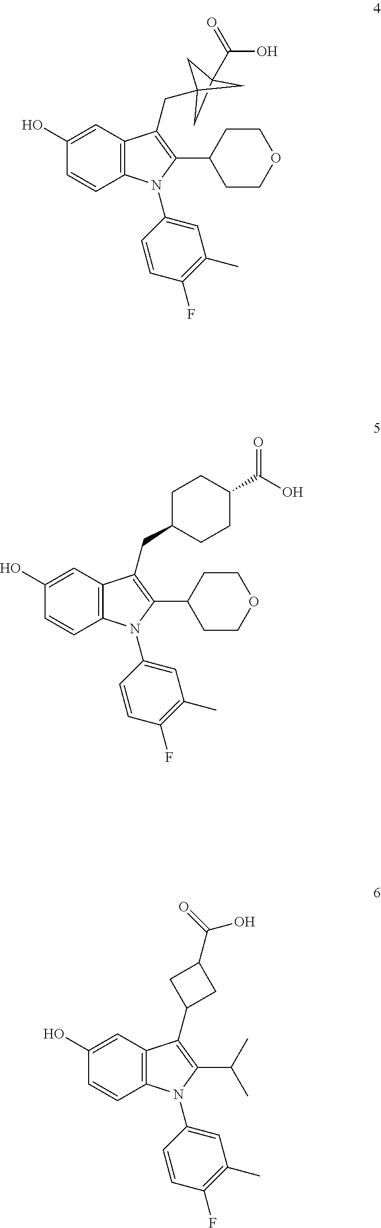 [SMILES: Cc1cc(-n2c(C3CCOCC3)c(CC34CC(C(=O)O)(C3)C4)c3cc(O)ccc32)ccc1F ; Cc1cc(-n2c(C3CCOCC3)c(C[C@H]3CC[C@H](C(=O)O)CC3)c3cc(O)ccc32)ccc1F ; Cc1cc(-n2c(C(C)C)c(C3CC(C(=O)O)C3)c3cc(O)ccc32)ccc1F] 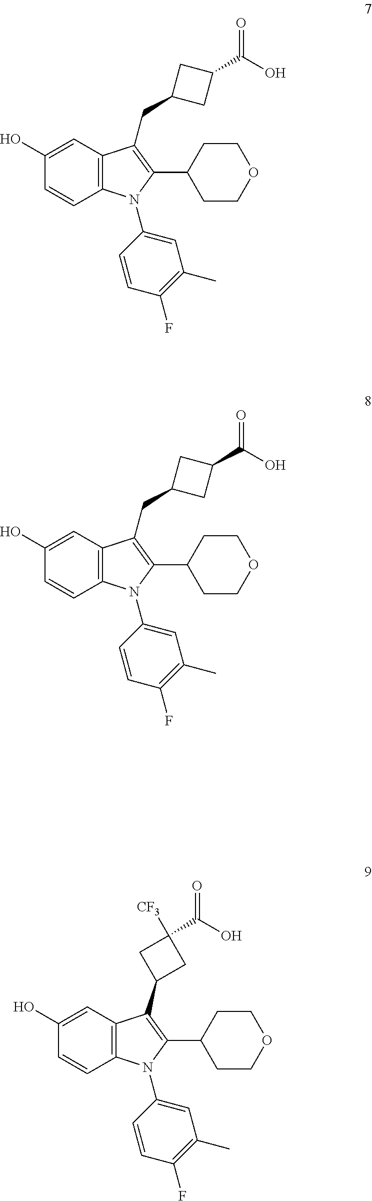 [SMILES: Cc1cc(-n2c(C3CCOCC3)c(C[C@H]3C[C@H](C(=O)O)C3)c3cc(O)ccc32)ccc1F ; Cc1cc(-n2c(C3CCOCC3)c(C[C@H]3C[C@@H](C(=O)O)C3)c3cc(O)ccc32)ccc1F ; Cc1cc(-n2c(C3CCOCC3)c([C@H]3C[C@@](C(=O)O)(C(F)(F)F)C3)c3cc(O)ccc32)ccc1F] 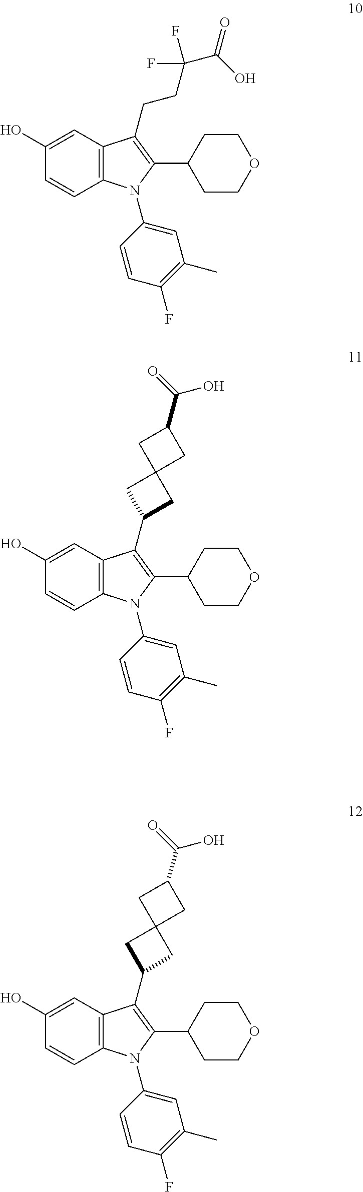 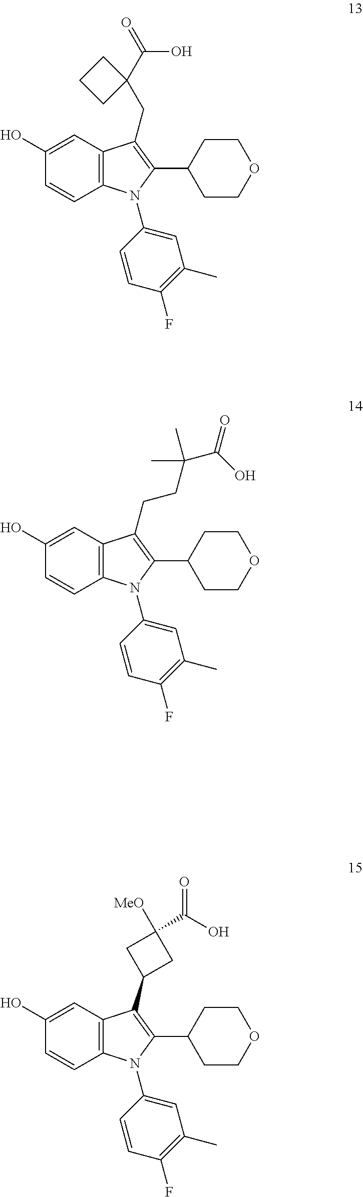 [SMILES: Cc1cc(-n2c(C3CCOCC3)c(CC3(C(=O)O)CCC3)c3cc(O)ccc32)ccc1F ; Cc1cc(-n2c(C3CCOCC3)c(CCC(C)(C)C(=O)O)c3cc(O)ccc32)ccc1F ; CO[C@]1(C(=O)O)C[C@H](c2c(C3CCOCC3)n(-c3ccc(F)c(C)c3)c3ccc(O)cc32)C1] 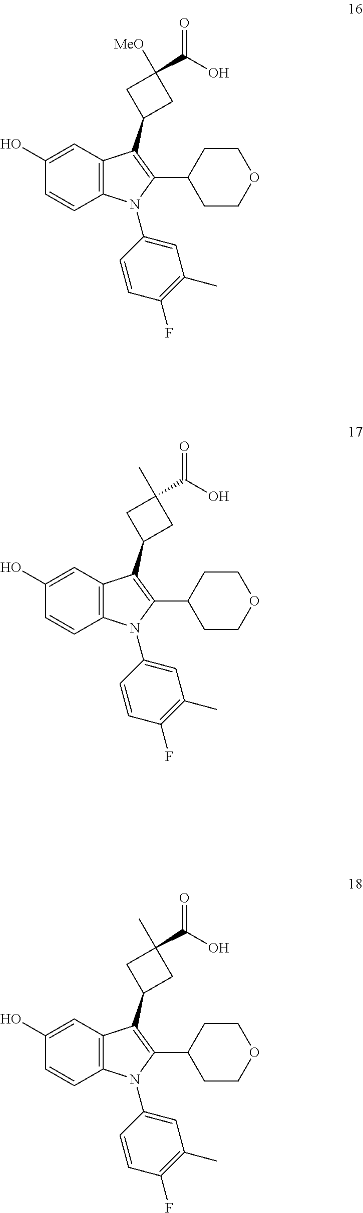 [SMILES: CO[C@]1(C(=O)O)C[C@@H](c2c(C3CCOCC3)n(-c3ccc(F)c(C)c3)c3ccc(O)cc32)C1 ; Cc1cc(-n2c(C3CCOCC3)c([C@H]3C[C@](C)(C(=O)O)C3)c3cc(O)ccc32)ccc1F ; Cc1cc(-n2c(C3CCOCC3)c([C@H]3C[C@@](C)(C(=O)O)C3)c3cc(O)ccc32)ccc1F] 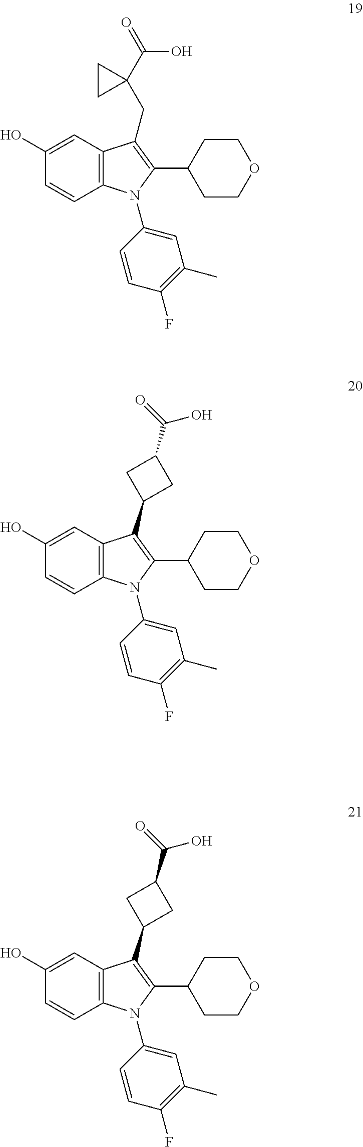 [SMILES: Cc1cc(-n2c(C3CCOCC3)c(CC3(C(=O)O)CC3)c3cc(O)ccc32)ccc1F ; Cc1cc(-n2c(C3CCOCC3)c([C@H]3C[C@H](C(=O)O)C3)c3cc(O)ccc32)ccc1F ; Cc1cc(-n2c(C3CCOCC3)c([C@H]3C[C@@H](C(=O)O)C3)c3cc(O)ccc32)ccc1F] 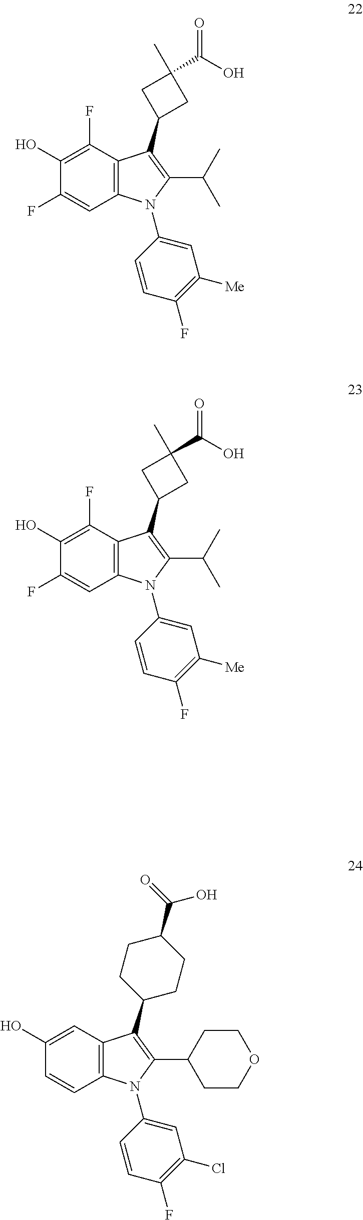 [SMILES: Cc1cc(-n2c(C(C)C)c([C@H]3C[C@](C)(C(=O)O)C3)c3c(F)c(O)c(F)cc32)ccc1F ; Cc1cc(-n2c(C(C)C)c([C@H]3C[C@@](C)(C(=O)O)C3)c3c(F)c(O)c(F)cc32)ccc1F ; O=C(O)[C@H]1CC[C@@H](c2c(C3CCOCC3)n(-c3ccc(F)c(Cl)c3)c3ccc(O)cc32)CC1] 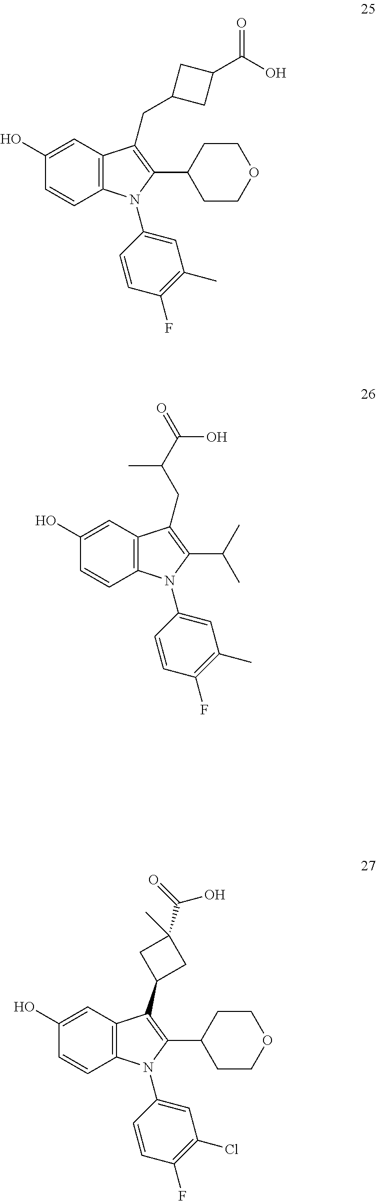 [SMILES: Cc1cc(-n2c(C3CCOCC3)c(CC3CC(C(=O)O)C3)c3cc(O)ccc32)ccc1F ; Cc1cc(-n2c(C(C)C)c(CC(C)C(=O)O)c3cc(O)ccc32)ccc1F ; C[C@]1(C(=O)O)C[C@H](c2c(C3CCOCC3)n(-c3ccc(F)c(Cl)c3)c3ccc(O)cc32)C1] 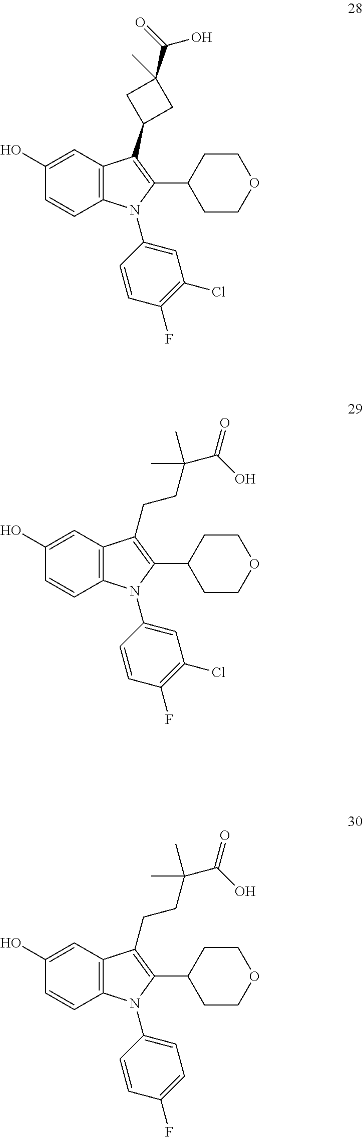 [SMILES: C[C@]1(C(=O)O)C[C@@H](c2c(C3CCOCC3)n(-c3ccc(F)c(Cl)c3)c3ccc(O)cc32)C1 ; CC(C)(CCc1c(C2CCOCC2)n(-c2ccc(F)c(Cl)c2)c2ccc(O)cc12)C(=O)O ; CC(C)(CCc1c(C2CCOCC2)n(-c2ccc(F)cc2)c2ccc(O)cc12)C(=O)O] 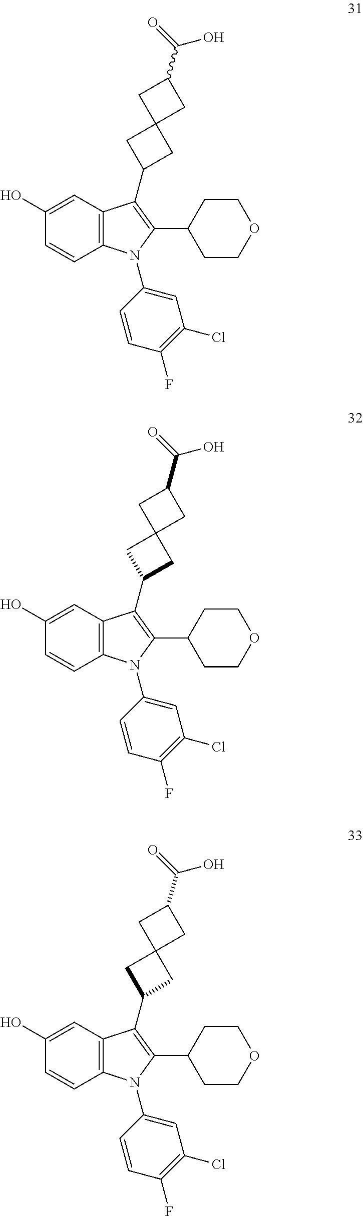 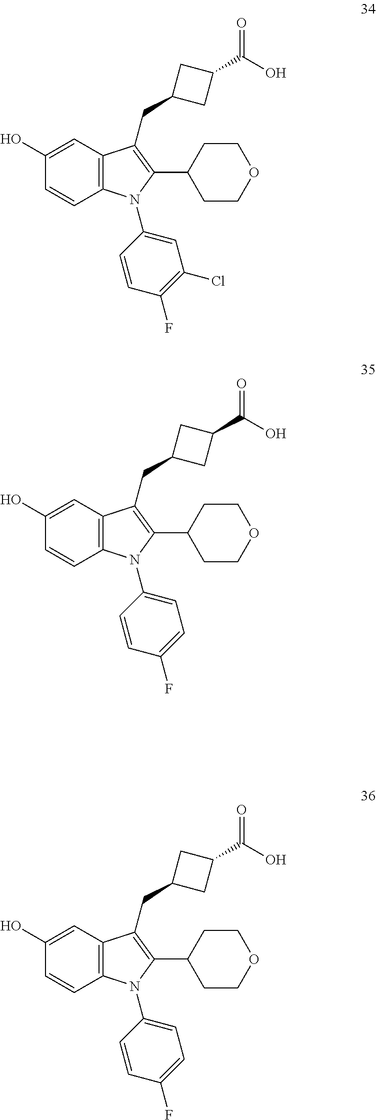 [SMILES: O=C(O)[C@H]1C[C@H](Cc2c(C3CCOCC3)n(-c3ccc(F)c(Cl)c3)c3ccc(O)cc23)C1 ; O=C(O)[C@H]1C[C@@H](Cc2c(C3CCOCC3)n(-c3ccc(F)cc3)c3ccc(O)cc23)C1 ; O=C(O)[C@H]1C[C@H](Cc2c(C3CCOCC3)n(-c3ccc(F)cc3)c3ccc(O)cc23)C1] 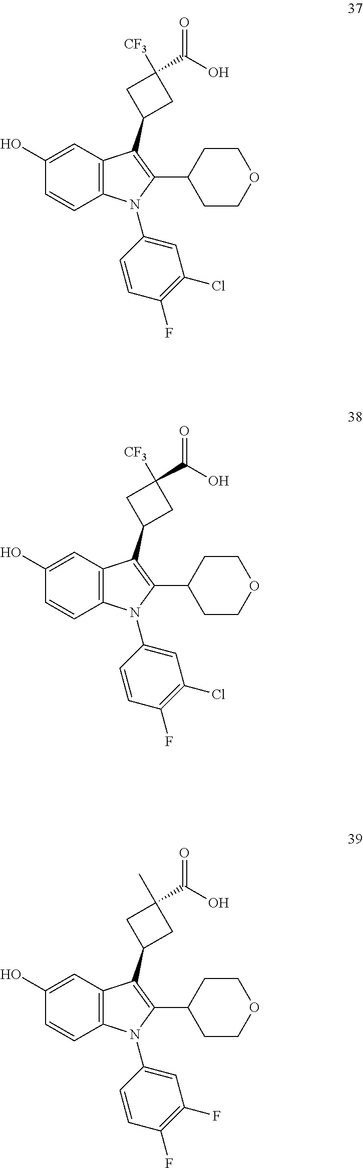 [SMILES: O=C(O)[C@]1(C(F)(F)F)C[C@@H](c2c(C3CCOCC3)n(-c3ccc(F)c(Cl)c3)c3ccc(O)cc32)C1 ; O=C(O)[C@]1(C(F)(F)F)C[C@H](c2c(C3CCOCC3)n(-c3ccc(F)c(Cl)c3)c3ccc(O)cc32)C1 ; C[C@]1(C(=O)O)C[C@H](c2c(C3CCOCC3)n(-c3ccc(F)c(F)c3)c3ccc(O)cc32)C1] 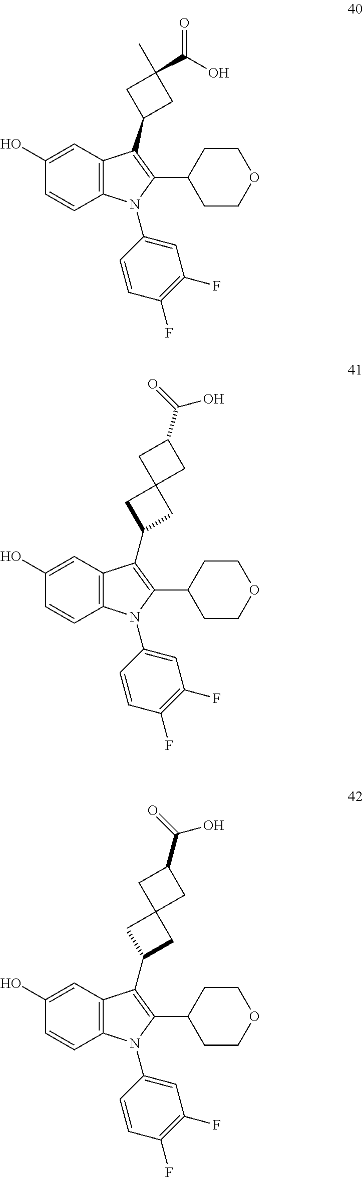 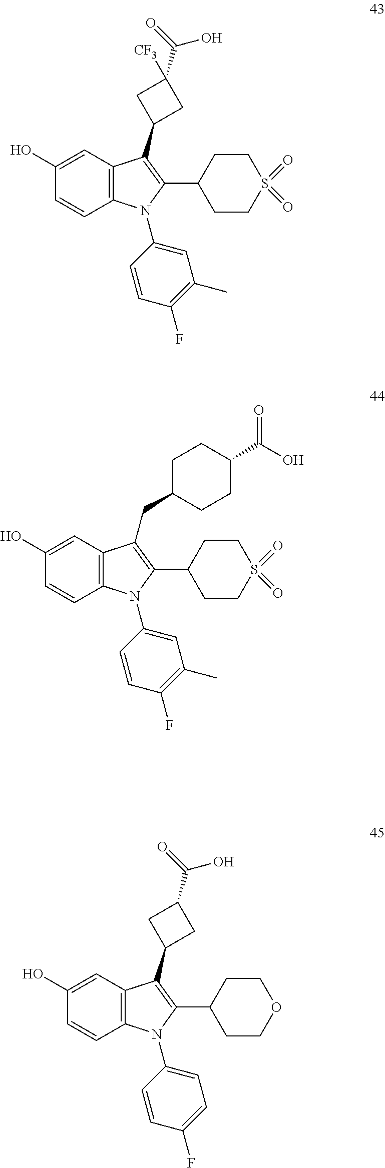 [SMILES: Cc1cc(-n2c(C3CCS(=O)(=O)CC3)c([C@H]3C[C@@](C(=O)O)(C(F)(F)F)C3)c3cc(O)ccc32)ccc1F ; Cc1cc(-n2c(C3CCS(=O)(=O)CC3)c(C[C@H]3CC[C@H](C(=O)O)CC3)c3cc(O)ccc32)ccc1F ; O=C(O)[C@H]1C[C@H](c2c(C3CCOCC3)n(-c3ccc(F)cc3)c3ccc(O)cc32)C1] 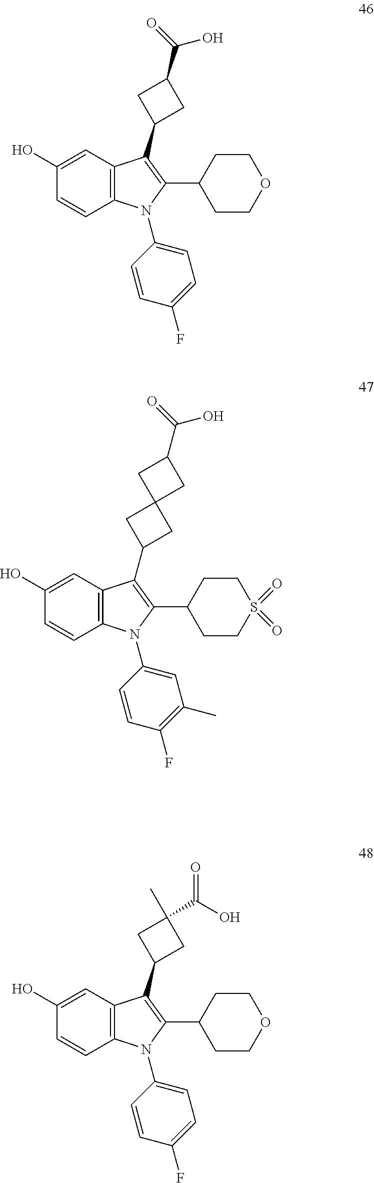 [SMILES: O=C(O)[C@H]1C[C@@H](c2c(C3CCOCC3)n(-c3ccc(F)cc3)c3ccc(O)cc32)C1 ; Cc1cc(-n2c(C3CCS(=O)(=O)CC3)c(C3CC4(CC(C(=O)O)C4)C3)c3cc(O)ccc32)ccc1F ; C[C@]1(C(=O)O)C[C@H](c2c(C3CCOCC3)n(-c3ccc(F)cc3)c3ccc(O)cc32)C1] 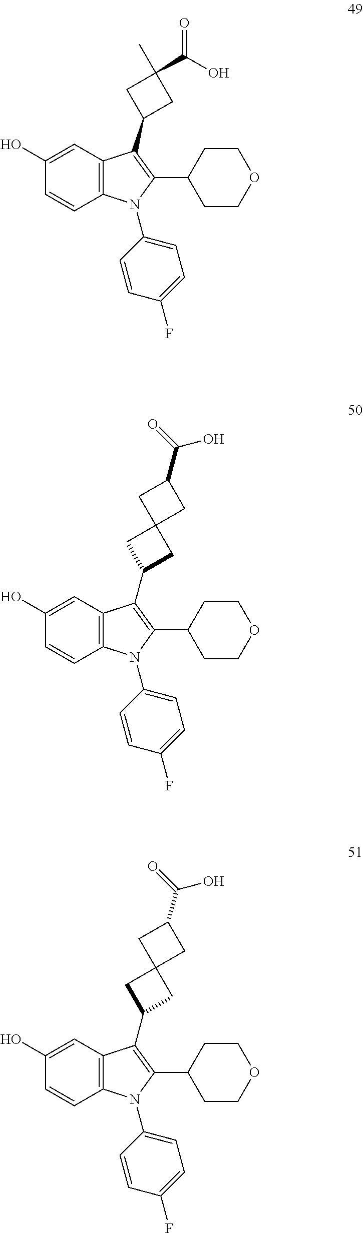 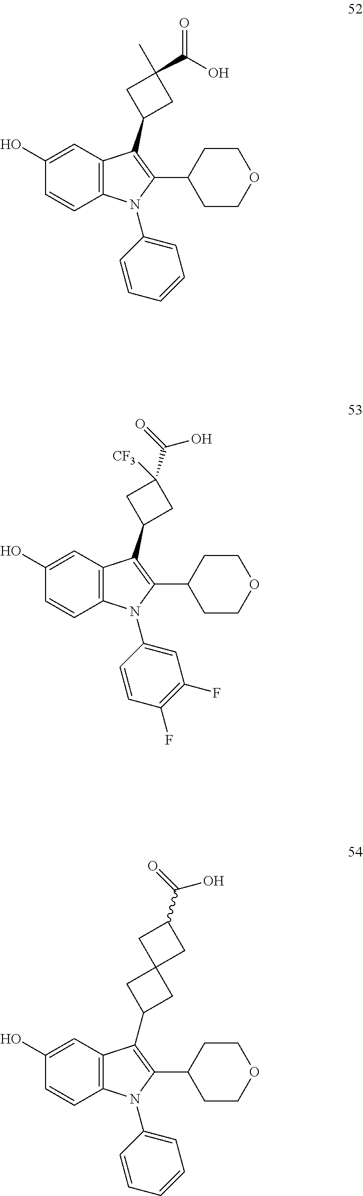 [SMILES: C[C@]1(C(=O)O)C[C@@H](c2c(C3CCOCC3)n(-c3ccccc3)c3ccc(O)cc32)C1 ; O=C(O)[C@]1(C(F)(F)F)C[C@@H](c2c(C3CCOCC3)n(-c3ccc(F)c(F)c3)c3ccc(O)cc32)C1 ; O=C(O)C1CC2(C1)CC(c1c(C3CCOCC3)n(-c3ccccc3)c3ccc(O)cc13)C2] 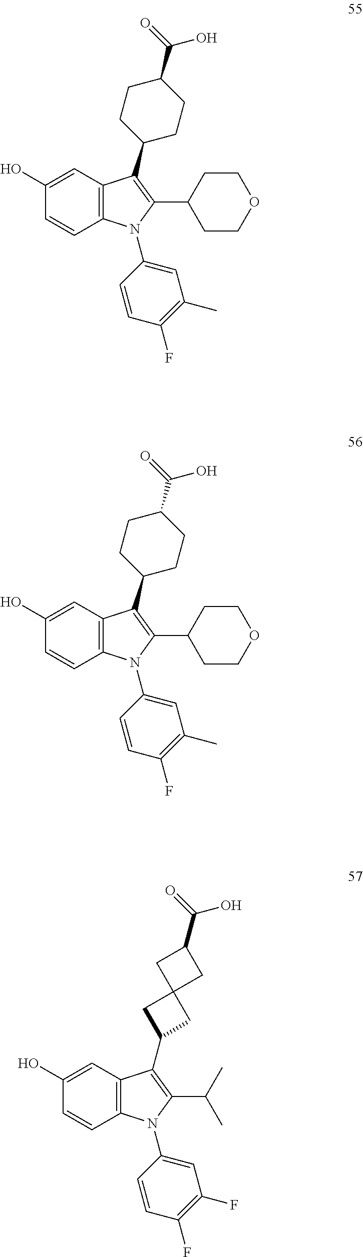 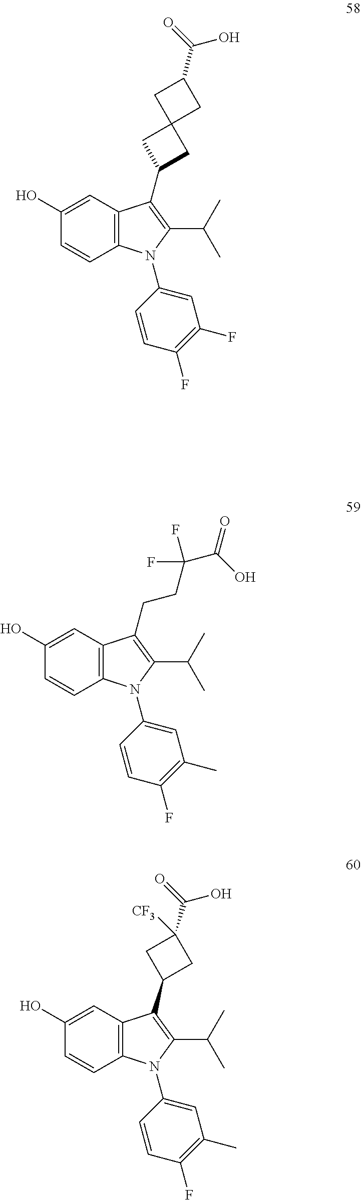 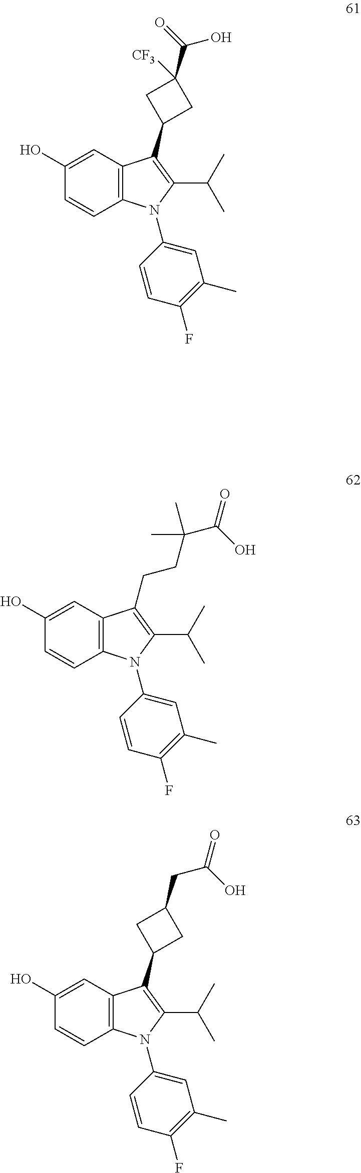 [SMILES: Cc1cc(-n2c(C(C)C)c([C@H]3C[C@](C(=O)O)(C(F)(F)F)C3)c3cc(O)ccc32)ccc1F ; Cc1cc(-n2c(C(C)C)c(CCC(C)(C)C(=O)O)c3cc(O)ccc32)ccc1F ; Cc1cc(-n2c(C(C)C)c([C@H]3C[C@@H](CC(=O)O)C3)c3cc(O)ccc32)ccc1F] 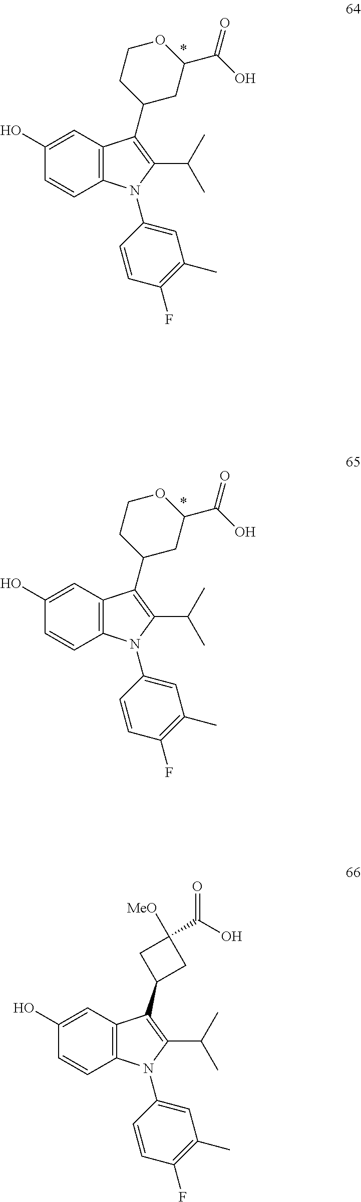 [SMILES: Cc1cc(-n2c(C(C)C)c(C3CCOC(C(=O)O)C3)c3cc(O)ccc32)ccc1F ; Cc1cc(-n2c(C(C)C)c(C3CCOC(C(=O)O)C3)c3cc(O)ccc32)ccc1F ; CO[C@]1(C(=O)O)C[C@H](c2c(C(C)C)n(-c3ccc(F)c(C)c3)c3ccc(O)cc32)C1] 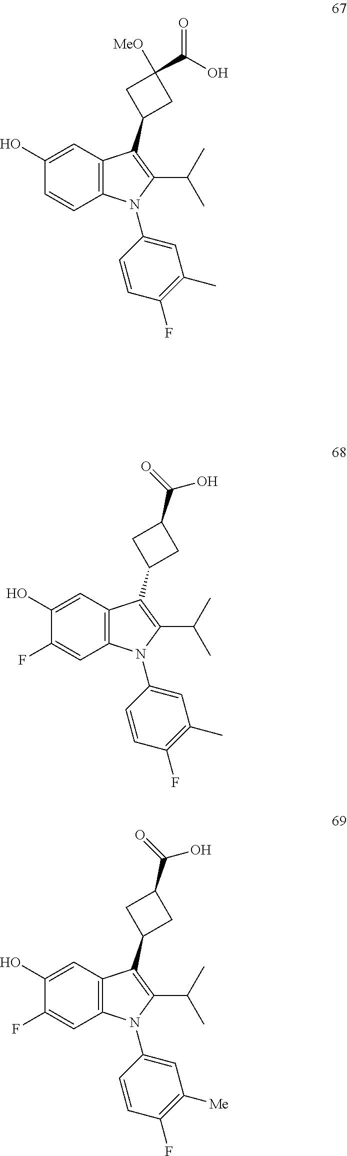 [SMILES: CO[C@]1(C(=O)O)C[C@@H](c2c(C(C)C)n(-c3ccc(F)c(C)c3)c3ccc(O)cc32)C1 ; Cc1cc(-n2c(C(C)C)c([C@H]3C[C@H](C(=O)O)C3)c3cc(O)c(F)cc32)ccc1F ; Cc1cc(-n2c(C(C)C)c([C@H]3C[C@@H](C(=O)O)C3)c3cc(O)c(F)cc32)ccc1F] 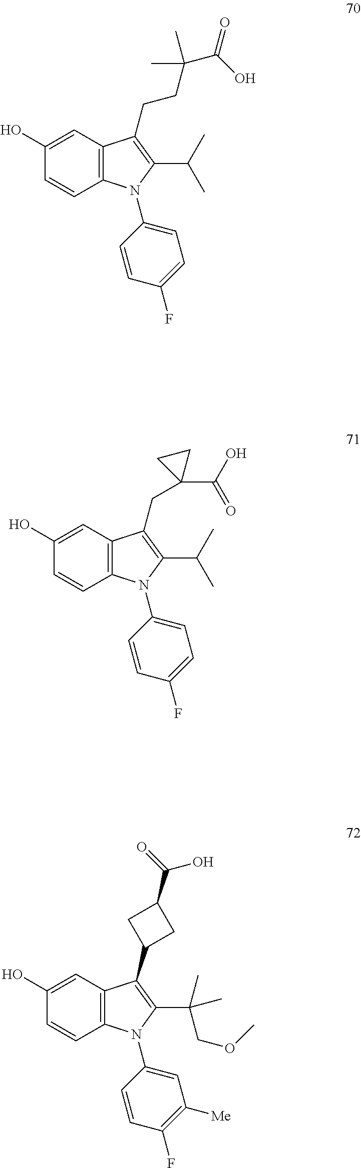 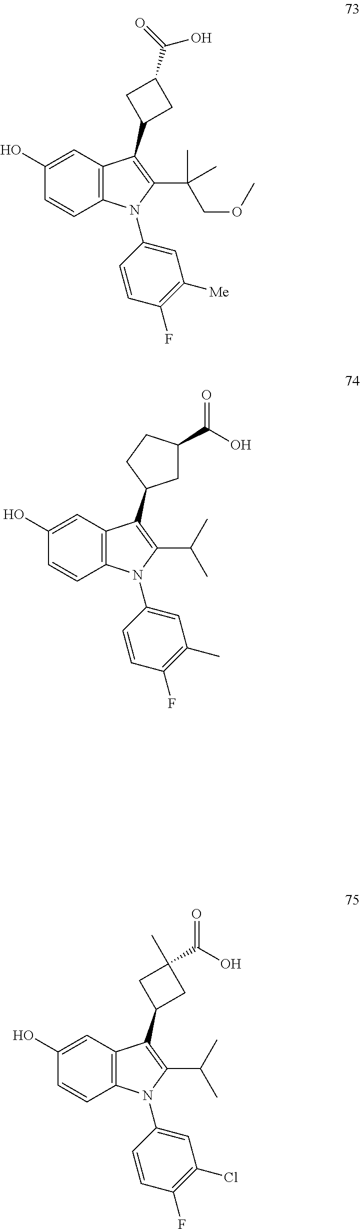 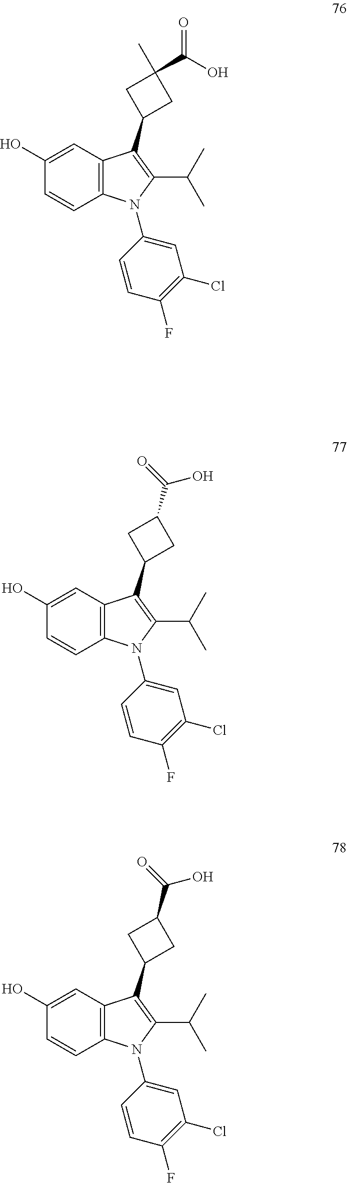 [SMILES: CC(C)c1c([C@H]2C[C@@](C)(C(=O)O)C2)c2cc(O)ccc2n1-c1ccc(F)c(Cl)c1 ; CC(C)c1c([C@H]2C[C@H](C(=O)O)C2)c2cc(O)ccc2n1-c1ccc(F)c(Cl)c1 ; CC(C)c1c([C@H]2C[C@@H](C(=O)O)C2)c2cc(O)ccc2n1-c1ccc(F)c(Cl)c1] 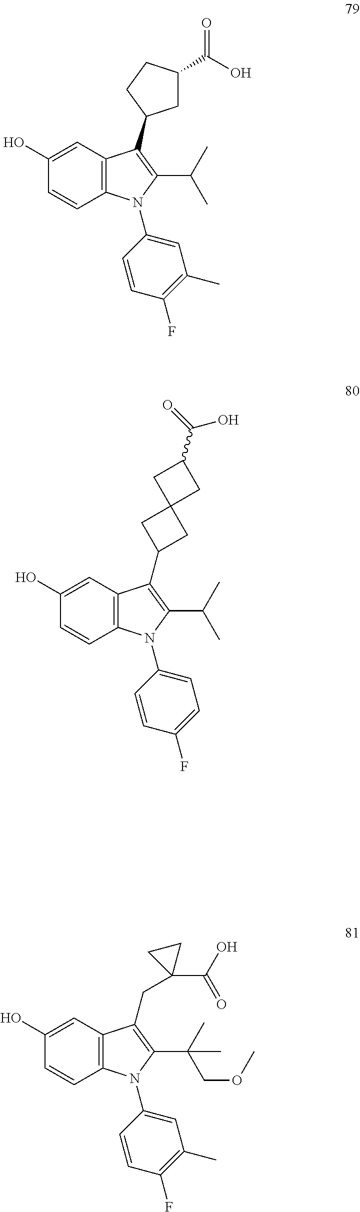 [SMILES: Cc1cc(-n2c(C(C)C)c([C@H]3CC[C@H](C(=O)O)C3)c3cc(O)ccc32)ccc1F ; CC(C)c1c(C2CC3(CC(C(=O)O)C3)C2)c2cc(O)ccc2n1-c1ccc(F)cc1 ; COCC(C)(C)c1c(CC2(C(=O)O)CC2)c2cc(O)ccc2n1-c1ccc(F)c(C)c1] 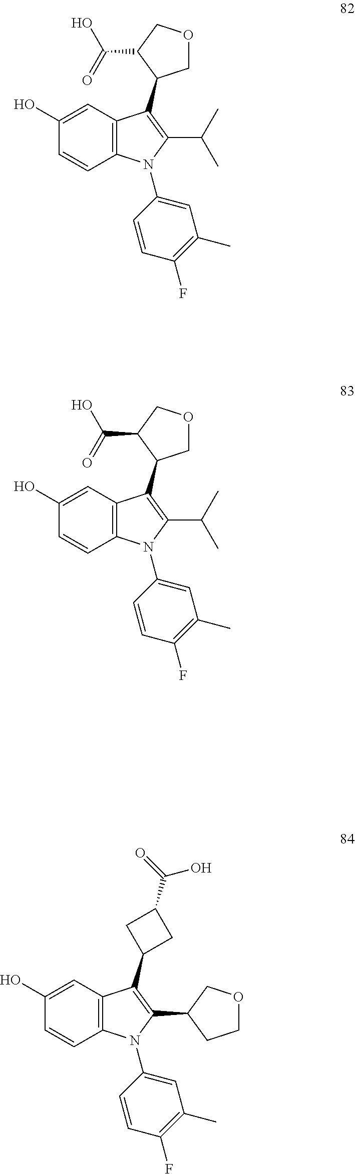 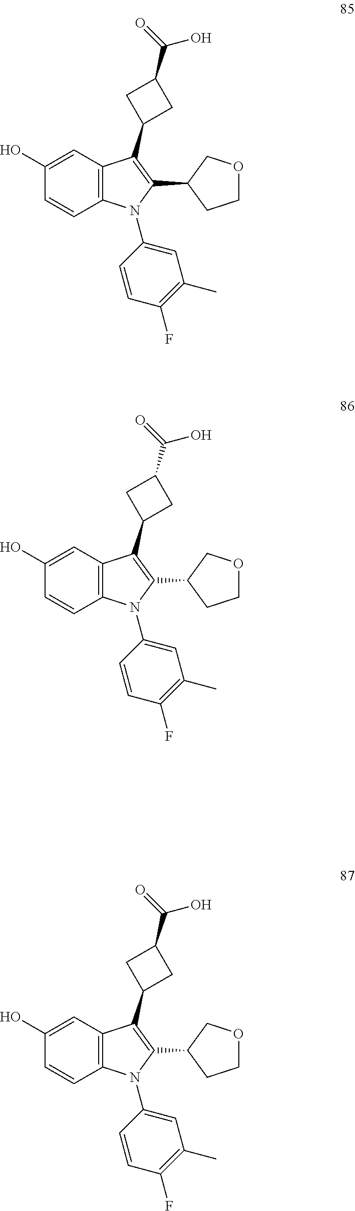 [SMILES: Cc1cc(-n2c([C@@H]3CCOC3)c([C@H]3C[C@@H](C(=O)O)C3)c3cc(O)ccc32)ccc1F ; Cc1cc(-n2c([C@H]3CCOC3)c([C@H]3C[C@H](C(=O)O)C3)c3cc(O)ccc32)ccc1F ; Cc1cc(-n2c([C@H]3CCOC3)c([C@H]3C[C@@H](C(=O)O)C3)c3cc(O)ccc32)ccc1F] 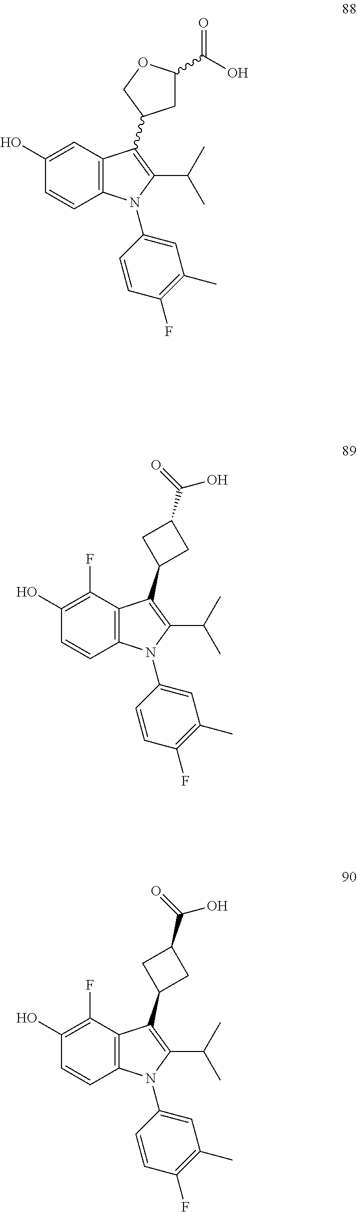 [SMILES: Cc1cc(-n2c(C(C)C)c(C3COC(C(=O)O)C3)c3cc(O)ccc32)ccc1F ; Cc1cc(-n2c(C(C)C)c([C@H]3C[C@H](C(=O)O)C3)c3c(F)c(O)ccc32)ccc1F ; Cc1cc(-n2c(C(C)C)c([C@H]3C[C@@H](C(=O)O)C3)c3c(F)c(O)ccc32)ccc1F] 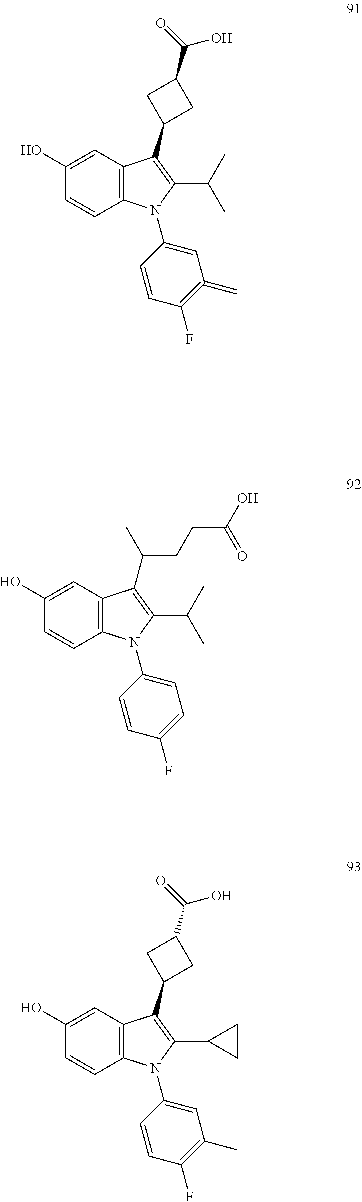 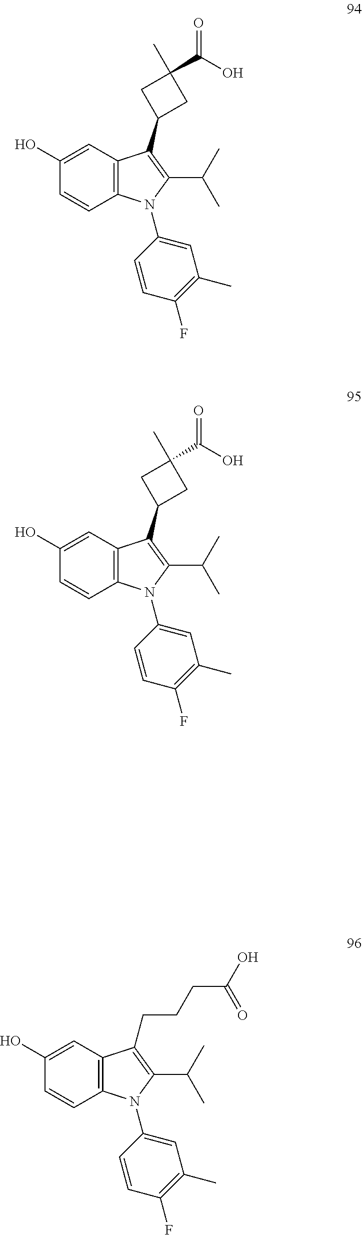 [SMILES: Cc1cc(-n2c(C(C)C)c([C@H]3C[C@@](C)(C(=O)O)C3)c3cc(O)ccc32)ccc1F ; Cc1cc(-n2c(C(C)C)c([C@H]3C[C@](C)(C(=O)O)C3)c3cc(O)ccc32)ccc1F ; Cc1cc(-n2c(C(C)C)c(CCCC(=O)O)c3cc(O)ccc32)ccc1F] 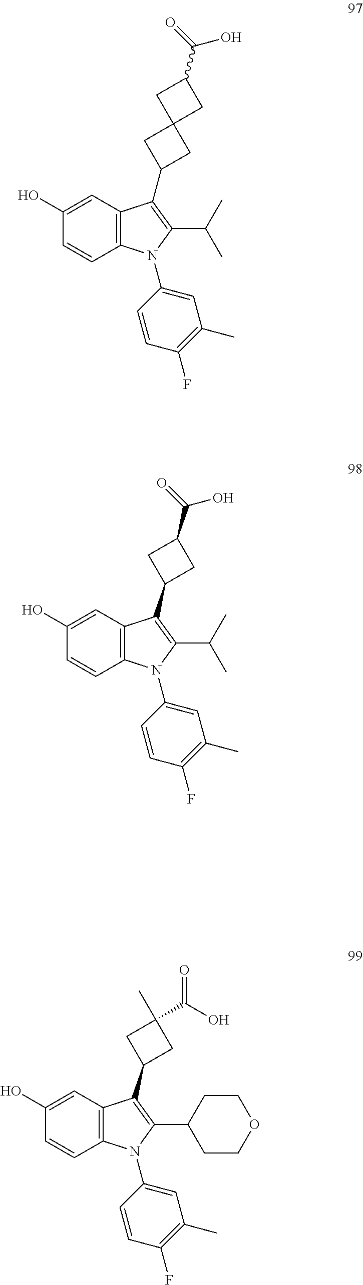 [SMILES: Cc1cc(-n2c(C(C)C)c(C3CC4(CC(C(=O)O)C4)C3)c3cc(O)ccc32)ccc1F ; Cc1cc(-n2c(C(C)C)c([C@H]3C[C@@H](C(=O)O)C3)c3cc(O)ccc32)ccc1F ; Cc1cc(-n2c(C3CCOCC3)c([C@H]3C[C@](C)(C(=O)O)C3)c3cc(O)ccc32)ccc1F] 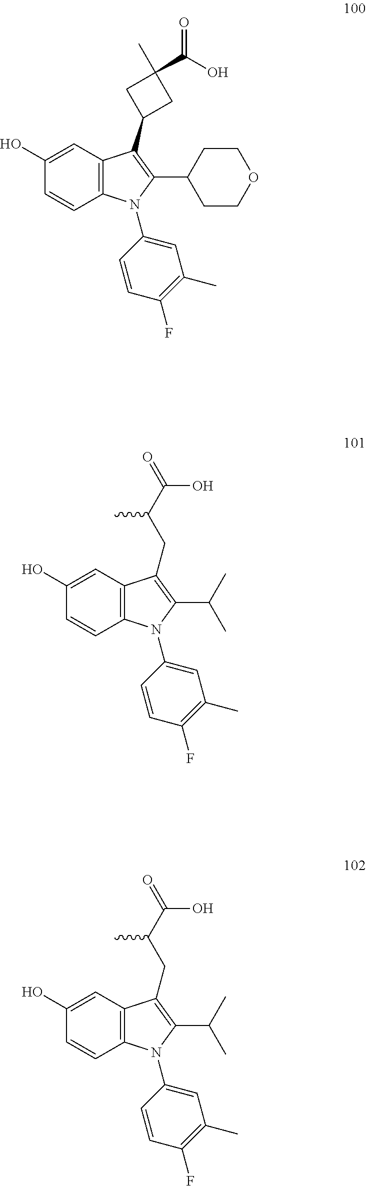 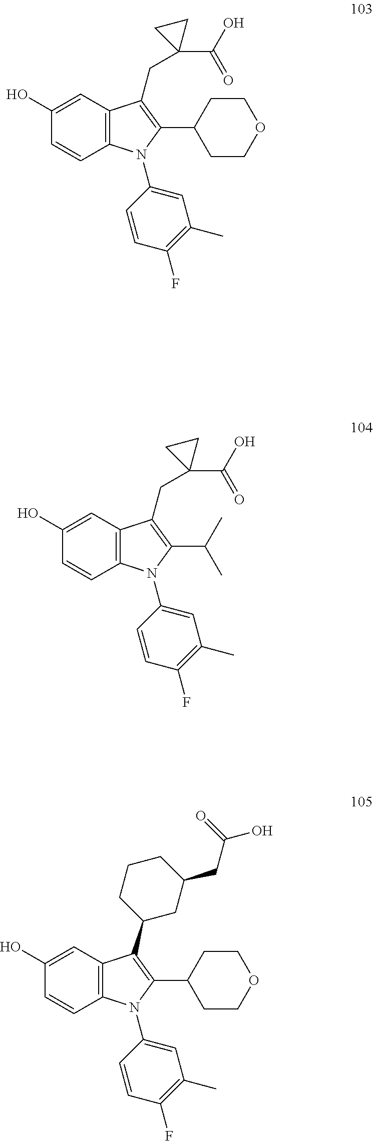 [SMILES: Cc1cc(-n2c(C3CCOCC3)c(CC3(C(=O)O)CC3)c3cc(O)ccc32)ccc1F ; Cc1cc(-n2c(C(C)C)c(CC3(C(=O)O)CC3)c3cc(O)ccc32)ccc1F ; Cc1cc(-n2c(C3CCOCC3)c([C@H]3CCC[C@@H](CC(=O)O)C3)c3cc(O)ccc32)ccc1F] 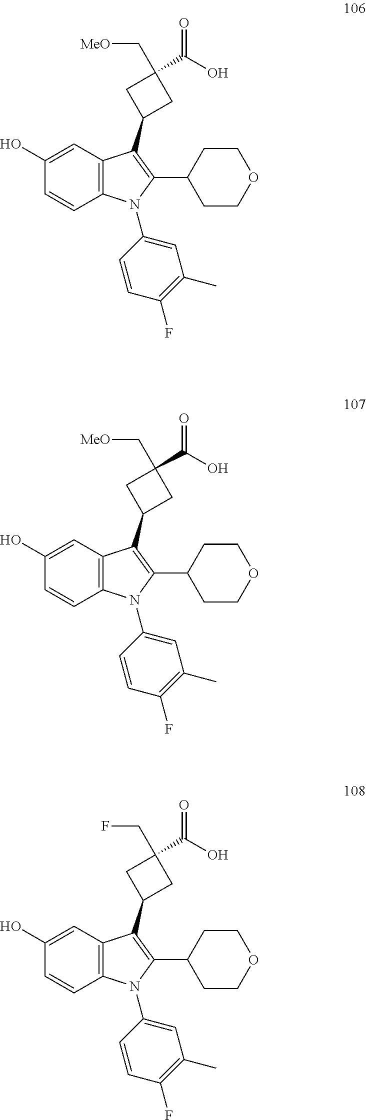 [SMILES: COC[C@]1(C(=O)O)C[C@H](c2c(C3CCOCC3)n(-c3ccc(F)c(C)c3)c3ccc(O)cc32)C1 ; COC[C@]1(C(=O)O)C[C@@H](c2c(C3CCOCC3)n(-c3ccc(F)c(C)c3)c3ccc(O)cc32)C1 ; Cc1cc(-n2c(C3CCOCC3)c([C@H]3C[C@](CF)(C(=O)O)C3)c3cc(O)ccc32)ccc1F] 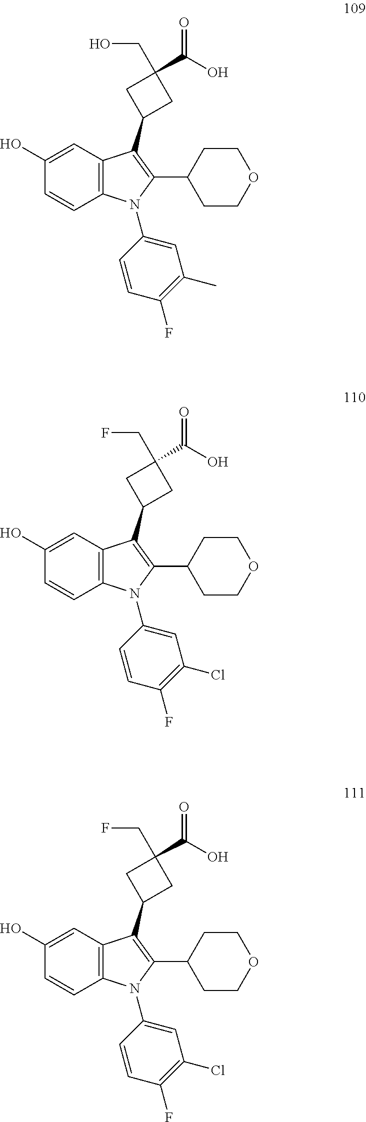 [SMILES: Cc1cc(-n2c(C3CCOCC3)c([C@H]3C[C@@](CO)(C(=O)O)C3)c3cc(O)ccc32)ccc1F ; O=C(O)[C@]1(CF)C[C@@H](c2c(C3CCOCC3)n(-c3ccc(F)c(Cl)c3)c3ccc(O)cc32)C1 ; O=C(O)[C@]1(CF)C[C@H](c2c(C3CCOCC3)n(-c3ccc(F)c(Cl)c3)c3ccc(O)cc32)C1] 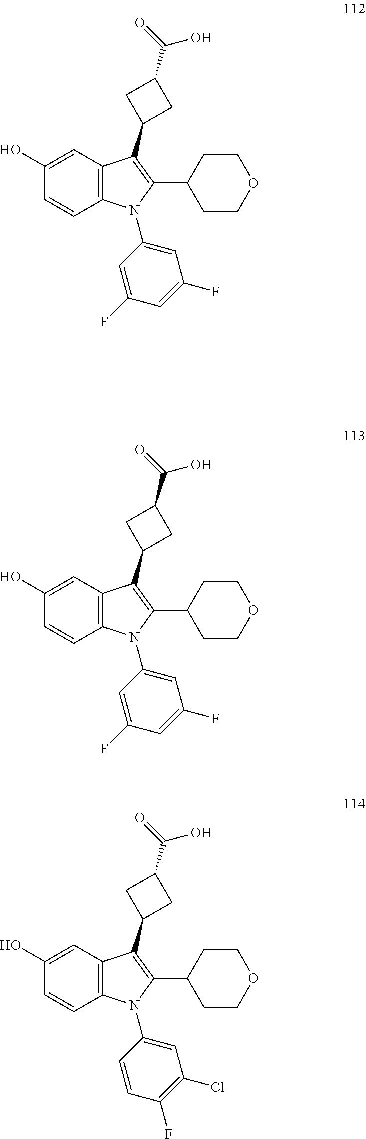 [SMILES: O=C(O)[C@H]1C[C@H](c2c(C3CCOCC3)n(-c3cc(F)cc(F)c3)c3ccc(O)cc32)C1 ; O=C(O)[C@H]1C[C@@H](c2c(C3CCOCC3)n(-c3cc(F)cc(F)c3)c3ccc(O)cc32)C1 ; O=C(O)[C@H]1C[C@H](c2c(C3CCOCC3)n(-c3ccc(F)c(Cl)c3)c3ccc(O)cc32)C1] 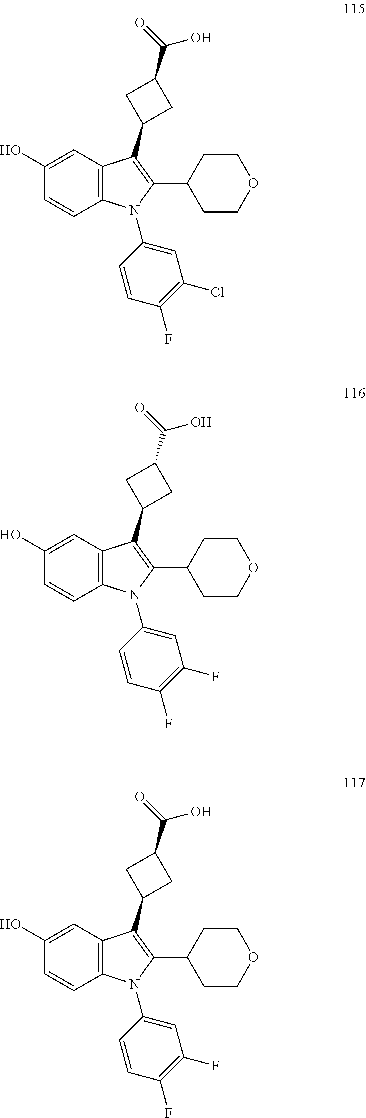 [SMILES: O=C(O)[C@H]1C[C@@H](c2c(C3CCOCC3)n(-c3ccc(F)c(Cl)c3)c3ccc(O)cc32)C1 ; O=C(O)[C@H]1C[C@H](c2c(C3CCOCC3)n(-c3ccc(F)c(F)c3)c3ccc(O)cc32)C1 ; O=C(O)[C@H]1C[C@@H](c2c(C3CCOCC3)n(-c3ccc(F)c(F)c3)c3ccc(O)cc32)C1] 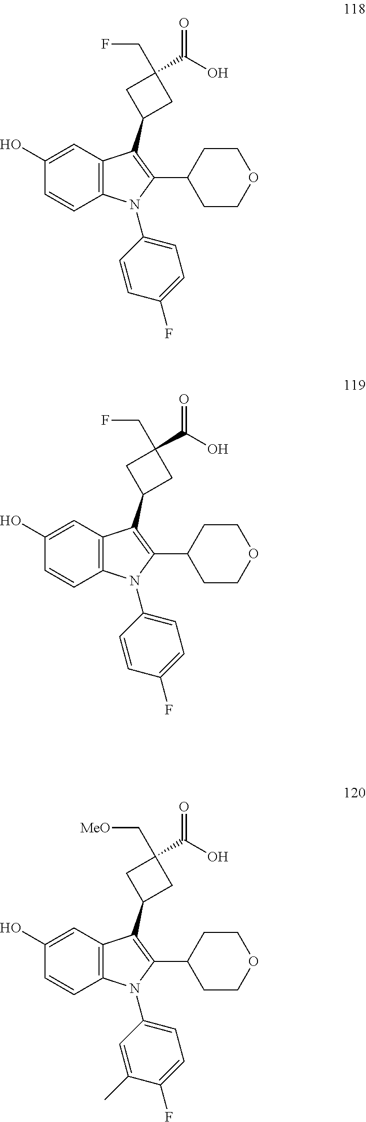 [SMILES: O=C(O)[C@]1(CF)C[C@@H](c2c(C3CCOCC3)n(-c3ccc(F)cc3)c3ccc(O)cc32)C1 ; O=C(O)[C@]1(CF)C[C@H](c2c(C3CCOCC3)n(-c3ccc(F)cc3)c3ccc(O)cc32)C1 ; COC[C@]1(C(=O)O)C[C@H](c2c(C3CCOCC3)n(-c3ccc(F)c(C)c3)c3ccc(O)cc32)C1] 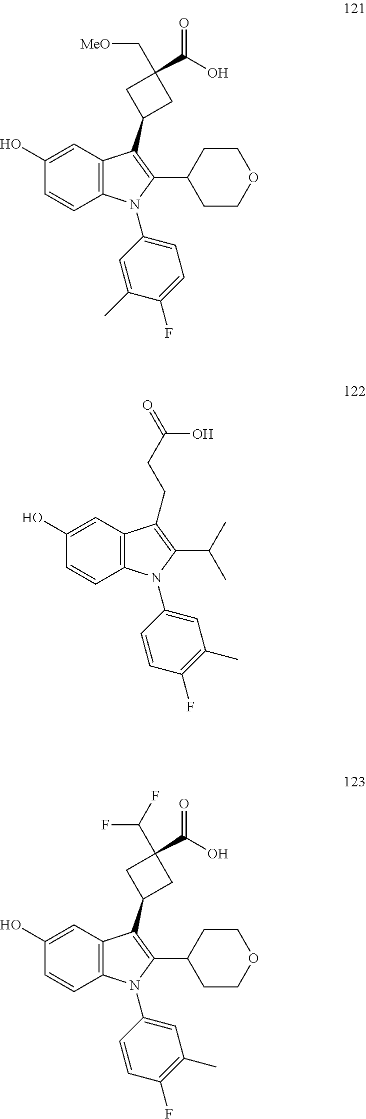 [SMILES: COC[C@]1(C(=O)O)C[C@@H](c2c(C3CCOCC3)n(-c3ccc(F)c(C)c3)c3ccc(O)cc32)C1 ; Cc1cc(-n2c(C(C)C)c(CCC(=O)O)c3cc(O)ccc32)ccc1F ; Cc1cc(-n2c(C3CCOCC3)c([C@H]3C[C@](C(=O)O)(C(F)F)C3)c3cc(O)ccc32)ccc1F] 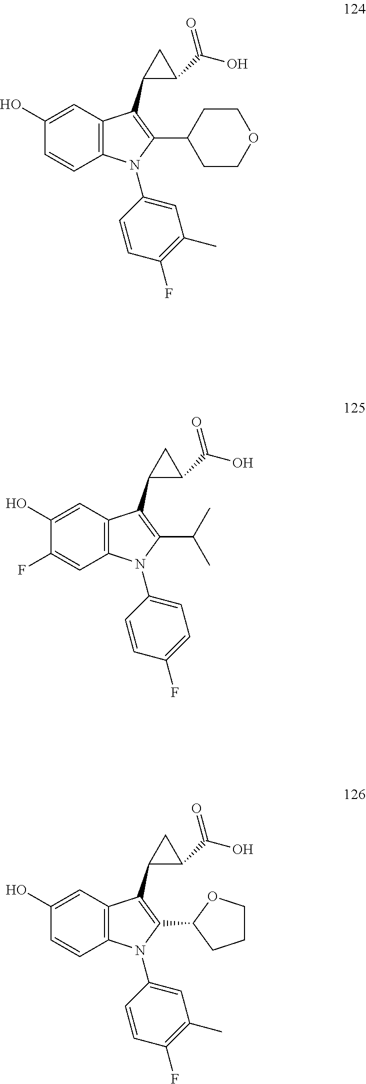 [SMILES: Cc1cc(-n2c(C3CCOCC3)c([C@H]3C[C@@H]3C(=O)O)c3cc(O)ccc32)ccc1F ; CC(C)c1c([C@H]2C[C@@H]2C(=O)O)c2cc(O)c(F)cc2n1-c1ccc(F)cc1 ; Cc1cc(-n2c([C@H]3CCCO3)c([C@H]3C[C@@H]3C(=O)O)c3cc(O)ccc32)ccc1F] 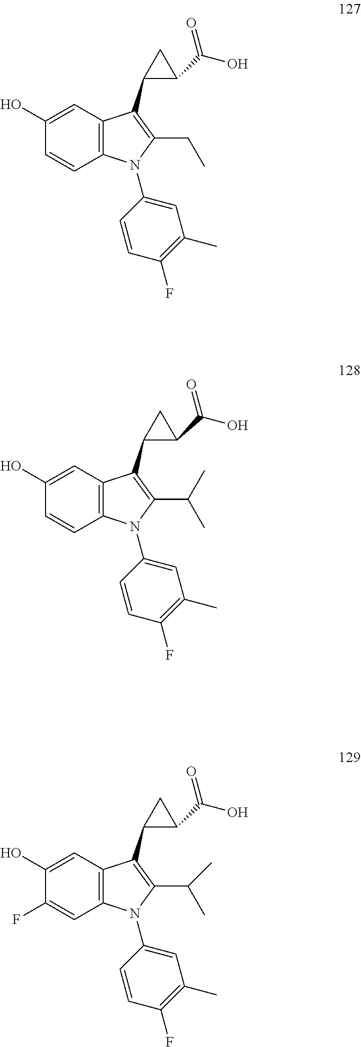 [SMILES: CCc1c([C@H]2C[C@@H]2C(=O)O)c2cc(O)ccc2n1-c1ccc(F)c(C)c1 ; Cc1cc(-n2c(C(C)C)c([C@H]3C[C@H]3C(=O)O)c3cc(O)ccc32)ccc1F ; Cc1cc(-n2c(C(C)C)c([C@H]3C[C@@H]3C(=O)O)c3cc(O)c(F)cc32)ccc1F] 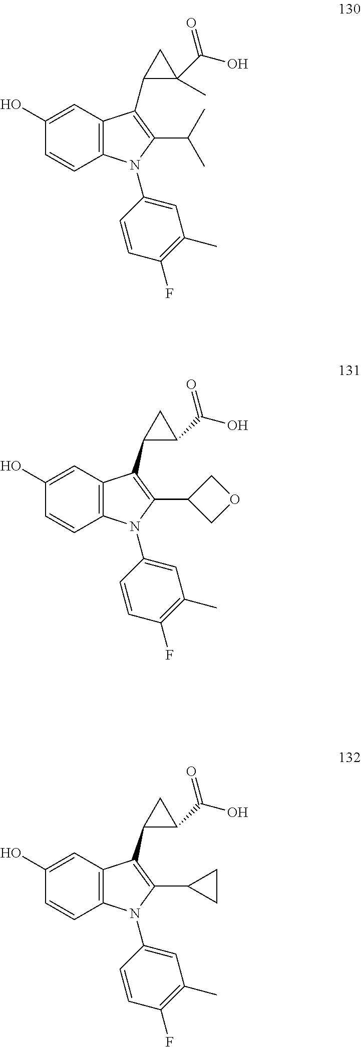 [SMILES: Cc1cc(-n2c(C(C)C)c(C3CC3(C)C(=O)O)c3cc(O)ccc32)ccc1F ; Cc1cc(-n2c(C3COC3)c([C@H]3C[C@@H]3C(=O)O)c3cc(O)ccc32)ccc1F ; Cc1cc(-n2c(C3CC3)c([C@H]3C[C@@H]3C(=O)O)c3cc(O)ccc32)ccc1F] 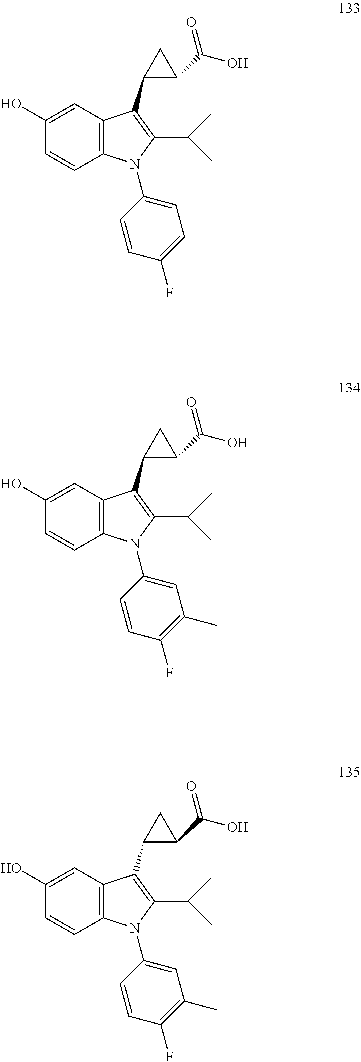 [SMILES: CC(C)c1c([C@H]2C[C@@H]2C(=O)O)c2cc(O)ccc2n1-c1ccc(F)cc1 ; Cc1cc(-n2c(C(C)C)c([C@H]3C[C@@H]3C(=O)O)c3cc(O)ccc32)ccc1F ; Cc1cc(-n2c(C(C)C)c([C@@H]3C[C@H]3C(=O)O)c3cc(O)ccc32)ccc1F] 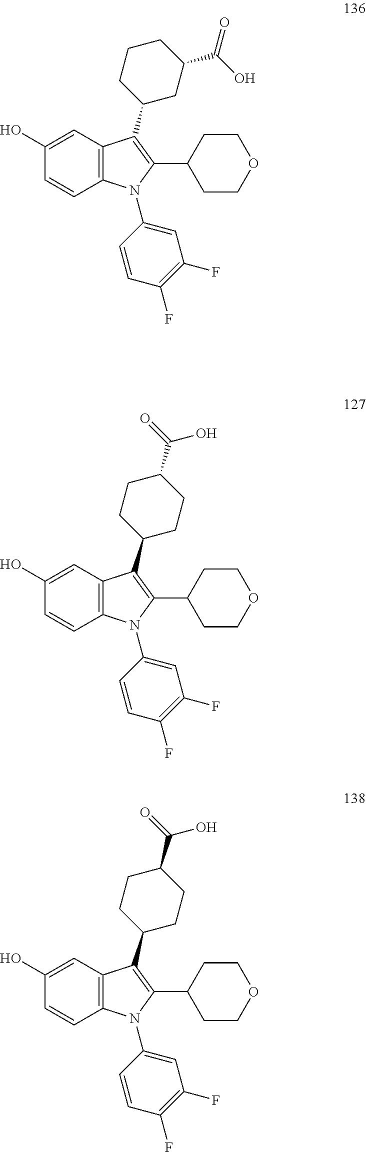 [SMILES: O=C(O)[C@H]1CCC[C@@H](c2c(C3CCOCC3)n(-c3ccc(F)c(F)c3)c3ccc(O)cc23)C1 ; O=C(O)[C@H]1CC[C@H](c2c(C3CCOCC3)n(-c3ccc(F)c(F)c3)c3ccc(O)cc32)CC1 ; O=C(O)[C@H]1CC[C@@H](c2c(C3CCOCC3)n(-c3ccc(F)c(F)c3)c3ccc(O)cc32)CC1] 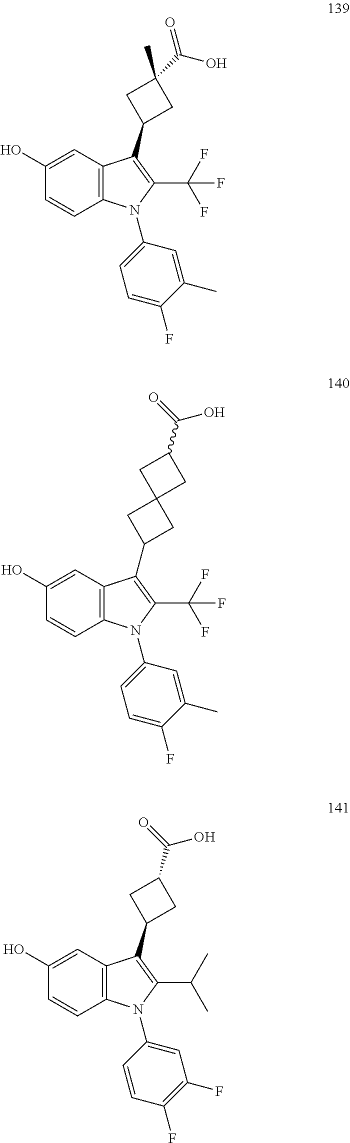 [SMILES: Cc1cc(-n2c(C(F)(F)F)c([C@H]3C[C@](C)(C(=O)O)C3)c3cc(O)ccc32)ccc1F ; Cc1cc(-n2c(C(F)(F)F)c(C3CC4(CC(C(=O)O)C4)C3)c3cc(O)ccc32)ccc1F ; CC(C)c1c([C@H]2C[C@H](C(=O)O)C2)c2cc(O)ccc2n1-c1ccc(F)c(F)c1] 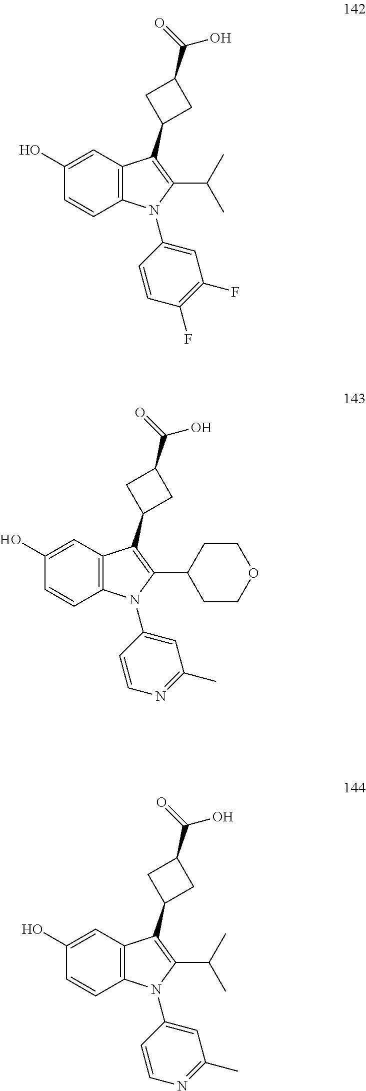 [SMILES: CC(C)c1c([C@H]2C[C@@H](C(=O)O)C2)c2cc(O)ccc2n1-c1ccc(F)c(F)c1 ; Cc1cc(-n2c(C3CCOCC3)c([C@H]3C[C@@H](C(=O)O)C3)c3cc(O)ccc32)ccn1 ; Cc1cc(-n2c(C(C)C)c([C@H]3C[C@@H](C(=O)O)C3)c3cc(O)ccc32)ccn1] 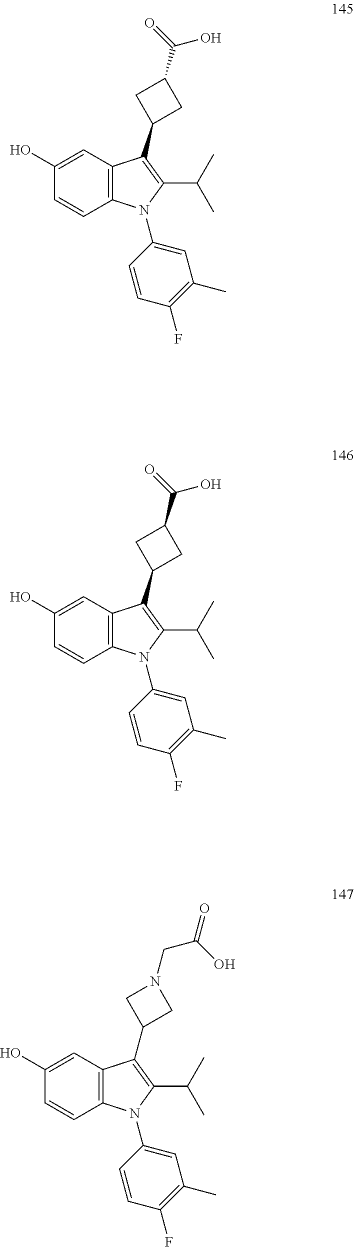 [SMILES: Cc1cc(-n2c(C(C)C)c([C@H]3C[C@H](C(=O)O)C3)c3cc(O)ccc32)ccc1F ; Cc1cc(-n2c(C(C)C)c([C@H]3C[C@@H](C(=O)O)C3)c3cc(O)ccc32)ccc1F ; Cc1cc(-n2c(C(C)C)c(C3CN(CC(=O)O)C3)c3cc(O)ccc32)ccc1F] 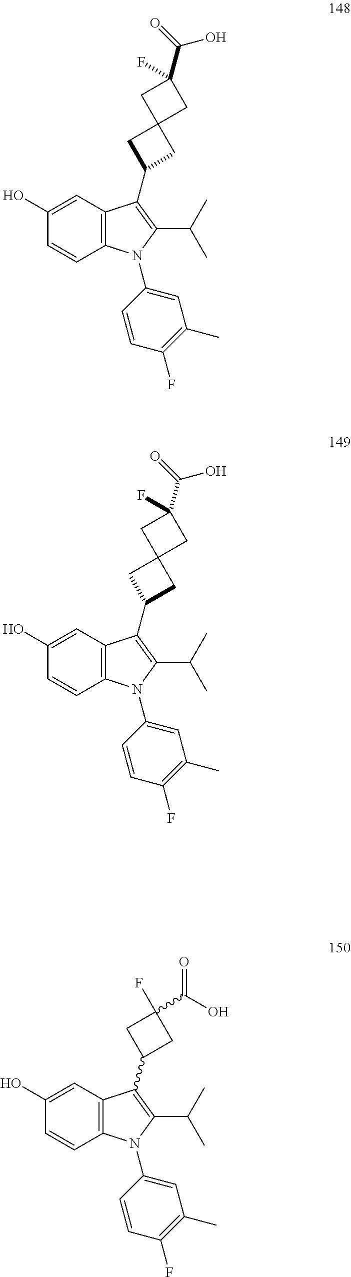 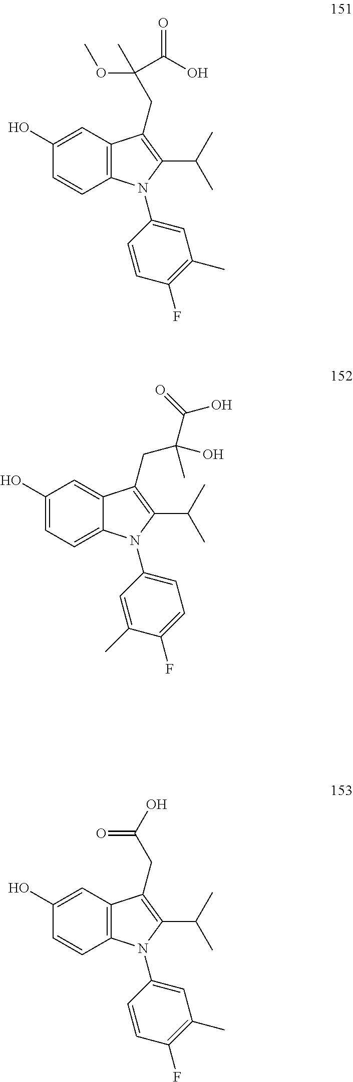 [SMILES: COC(C)(Cc1c(C(C)C)n(-c2ccc(F)c(C)c2)c2ccc(O)cc12)C(=O)O ; Cc1cc(-n2c(C(C)C)c(CC(C)(O)C(=O)O)c3cc(O)ccc32)ccc1F ; Cc1cc(-n2c(C(C)C)c(CC(=O)O)c3cc(O)ccc32)ccc1F] 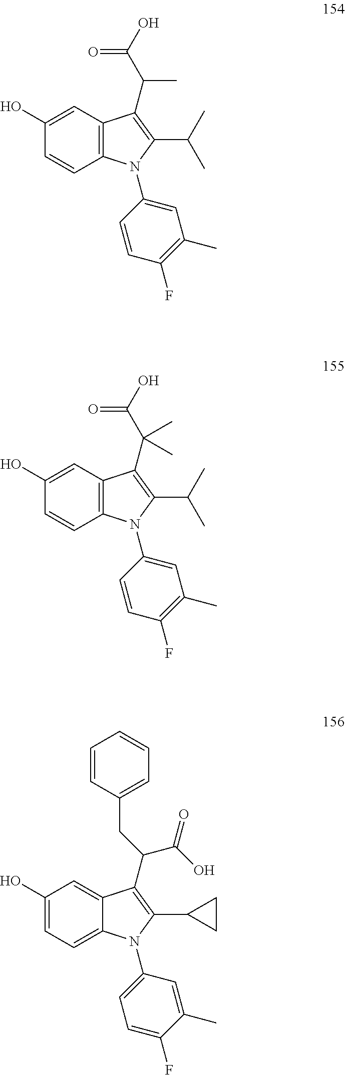 [SMILES: Cc1cc(-n2c(C(C)C)c(C(C)C(=O)O)c3cc(O)ccc32)ccc1F ; Cc1cc(-n2c(C(C)C)c(C(C)(C)C(=O)O)c3cc(O)ccc32)ccc1F ; Cc1cc(-n2c(C3CC3)c(C(Cc3ccccc3)C(=O)O)c3cc(O)ccc32)ccc1F] 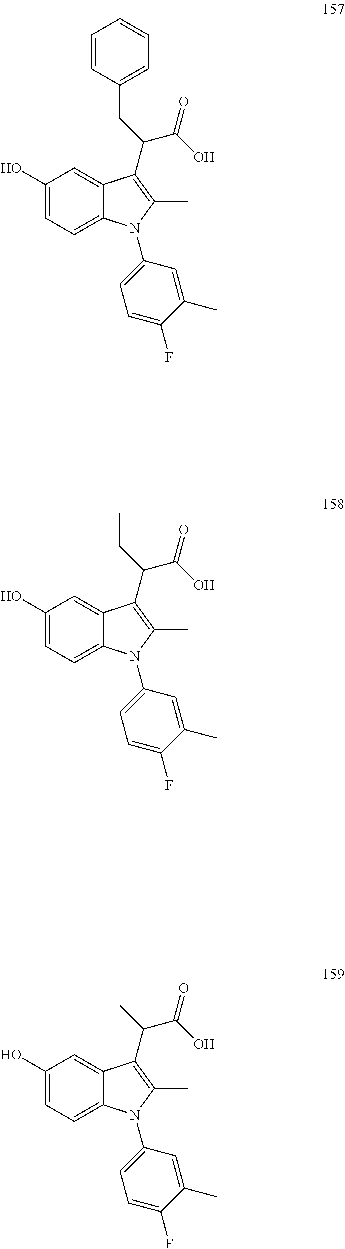 [SMILES: Cc1cc(-n2c(C)c(C(Cc3ccccc3)C(=O)O)c3cc(O)ccc32)ccc1F ; CCC(C(=O)O)c1c(C)n(-c2ccc(F)c(C)c2)c2ccc(O)cc12 ; Cc1cc(-n2c(C)c(C(C)C(=O)O)c3cc(O)ccc32)ccc1F] 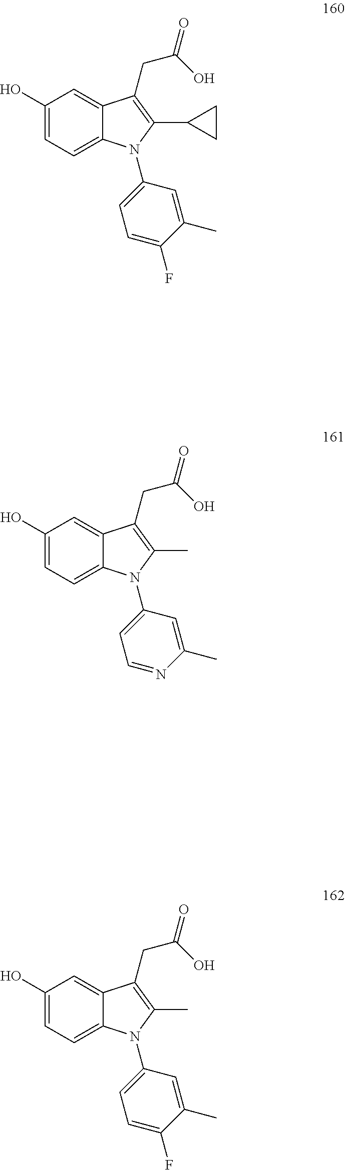 [SMILES: Cc1cc(-n2c(C3CC3)c(CC(=O)O)c3cc(O)ccc32)ccc1F ; Cc1cc(-n2c(C)c(CC(=O)O)c3cc(O)ccc32)ccn1 ; Cc1cc(-n2c(C)c(CC(=O)O)c3cc(O)ccc32)ccc1F] 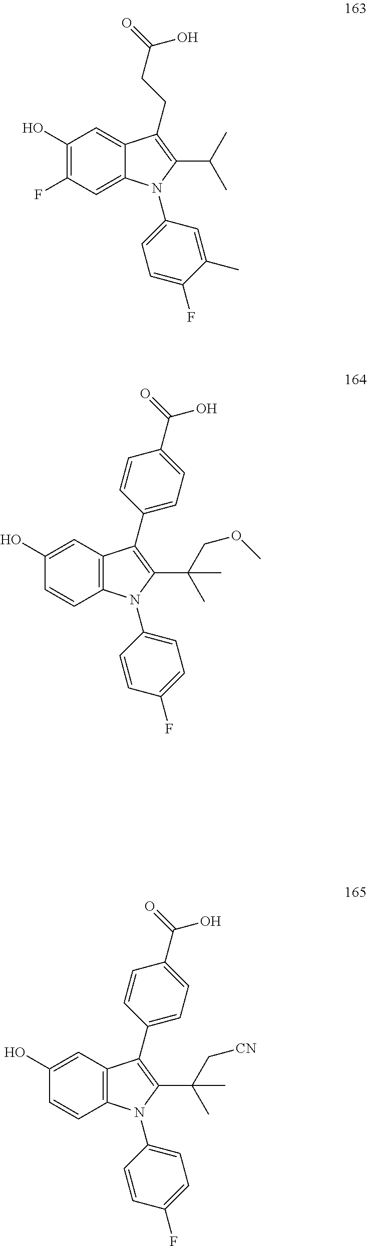 [SMILES: Cc1cc(-n2c(C(C)C)c(CCC(=O)O)c3cc(O)c(F)cc32)ccc1F ; COCC(C)(C)c1c(-c2ccc(C(=O)O)cc2)c2cc(O)ccc2n1-c1ccc(F)cc1 ; CC(C)(CC#N)c1c(-c2ccc(C(=O)O)cc2)c2cc(O)ccc2n1-c1ccc(F)cc1] 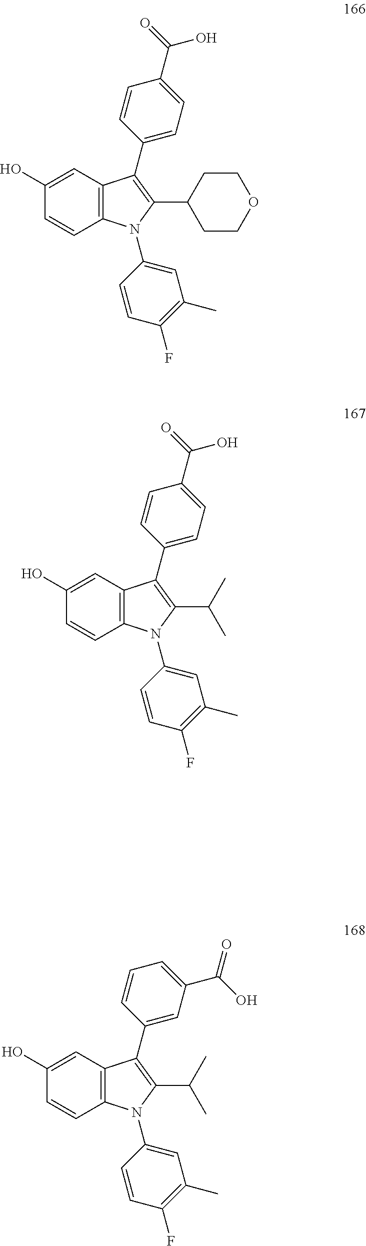 [SMILES: Cc1cc(-n2c(C3CCOCC3)c(-c3ccc(C(=O)O)cc3)c3cc(O)ccc32)ccc1F ; Cc1cc(-n2c(C(C)C)c(-c3ccc(C(=O)O)cc3)c3cc(O)ccc32)ccc1F ; Cc1cc(-n2c(C(C)C)c(-c3cccc(C(=O)O)c3)c3cc(O)ccc32)ccc1F] 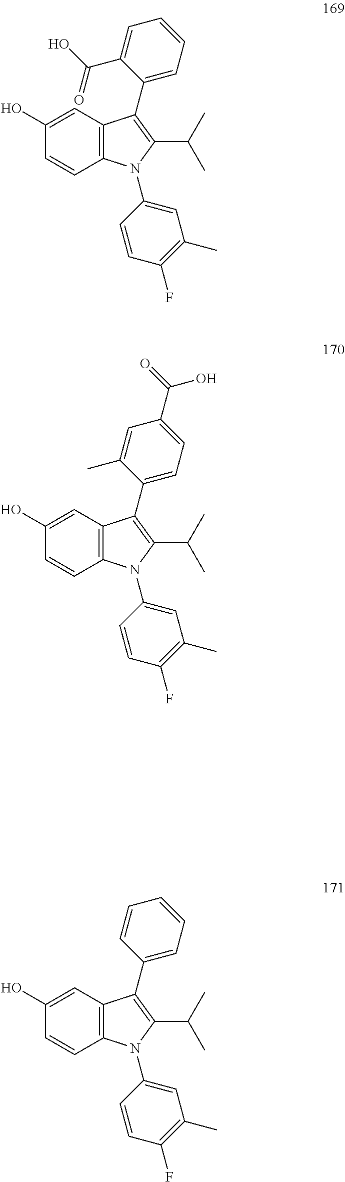 [SMILES: Cc1cc(-n2c(C(C)C)c(-c3ccccc3C(=O)O)c3cc(O)ccc32)ccc1F ; Cc1cc(-n2c(C(C)C)c(-c3ccc(C(=O)O)cc3C)c3cc(O)ccc32)ccc1F ; Cc1cc(-n2c(C(C)C)c(-c3ccccc3)c3cc(O)ccc32)ccc1F] 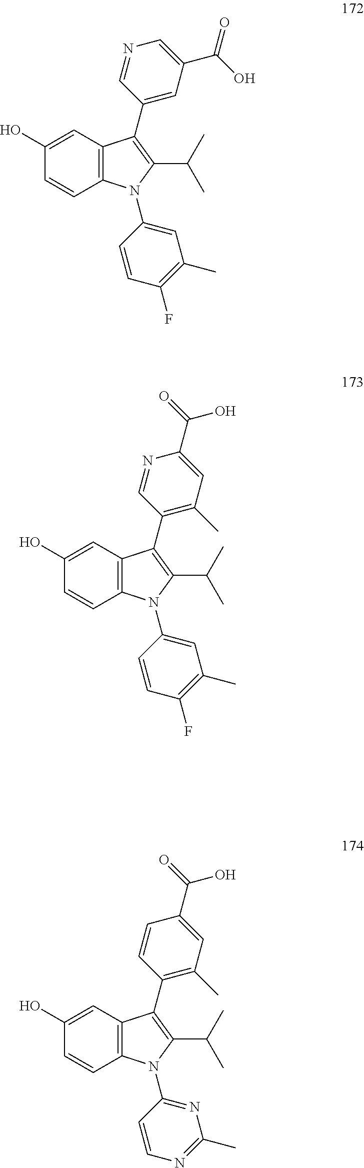 [SMILES: Cc1cc(-n2c(C(C)C)c(-c3cncc(C(=O)O)c3)c3cc(O)ccc32)ccc1F ; Cc1cc(-n2c(C(C)C)c(-c3cnc(C(=O)O)cc3C)c3cc(O)ccc32)ccc1F ; Cc1nccc(-n2c(C(C)C)c(-c3ccc(C(=O)O)cc3C)c3cc(O)ccc32)n1] 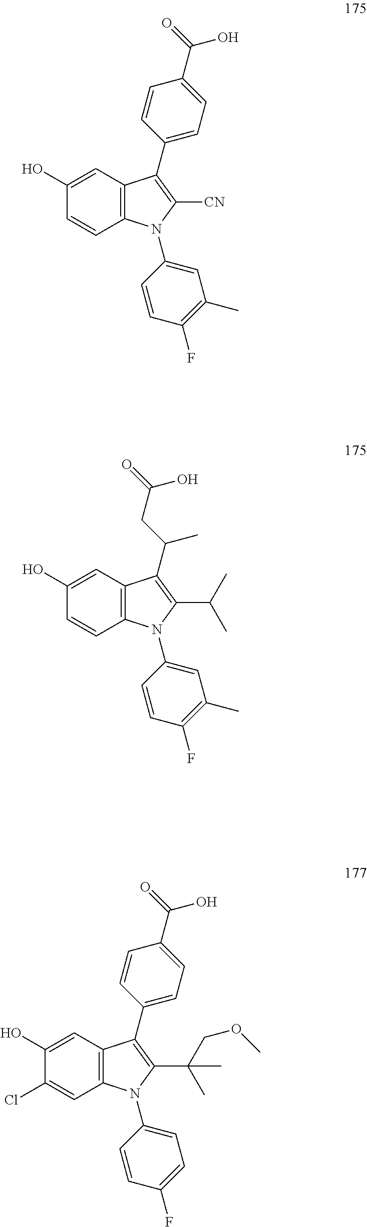 [SMILES: Cc1cc(-n2c(C#N)c(-c3ccc(C(=O)O)cc3)c3cc(O)ccc32)ccc1F ; Cc1cc(-n2c(C(C)C)c(C(C)CC(=O)O)c3cc(O)ccc32)ccc1F ; COCC(C)(C)c1c(-c2ccc(C(=O)O)cc2)c2cc(O)c(Cl)cc2n1-c1ccc(F)cc1] 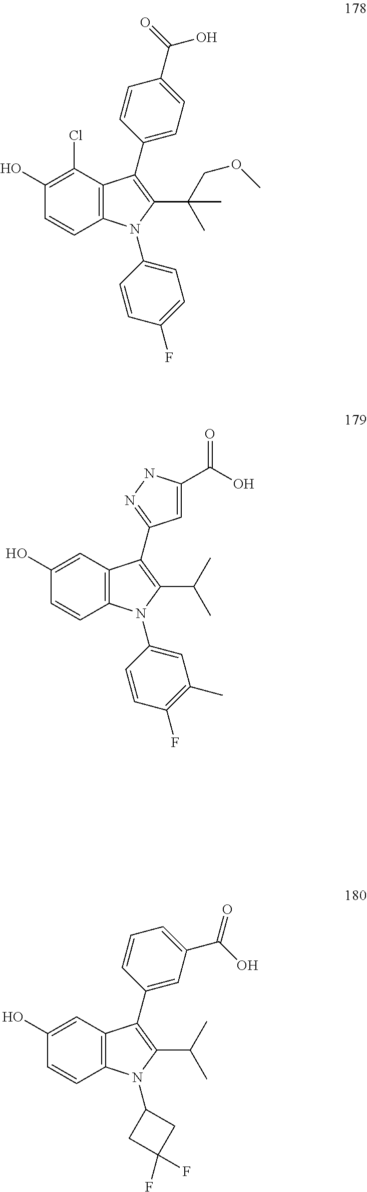 [SMILES: COCC(C)(C)c1c(-c2ccc(C(=O)O)cc2)c2c(Cl)c(O)ccc2n1-c1ccc(F)cc1 ; Cc1cc(-n2c(C(C)C)c(-c3cc(C(=O)O)[nH]n3)c3cc(O)ccc32)ccc1F ; CC(C)c1c(-c2cccc(C(=O)O)c2)c2cc(O)ccc2n1C1CC(F)(F)C1] 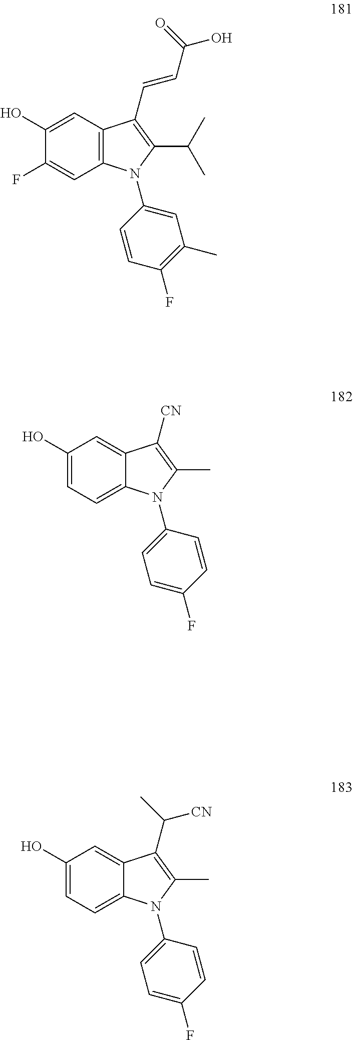 [SMILES: Cc1cc(-n2c(C(C)C)c(/C=C/C(=O)O)c3cc(O)c(F)cc32)ccc1F ; Cc1c(C#N)c2cc(O)ccc2n1-c1ccc(F)cc1 ; Cc1c(C(C)C#N)c2cc(O)ccc2n1-c1ccc(F)cc1] 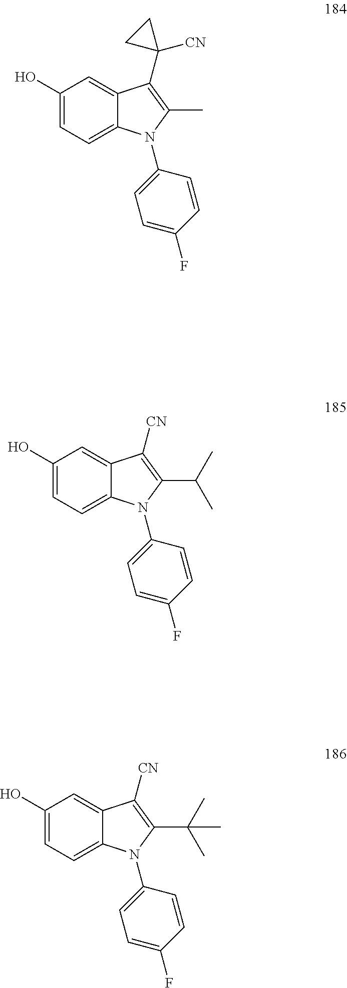 [SMILES: Cc1c(C2(C#N)CC2)c2cc(O)ccc2n1-c1ccc(F)cc1 ; CC(C)c1c(C#N)c2cc(O)ccc2n1-c1ccc(F)cc1 ; CC(C)(C)c1c(C#N)c2cc(O)ccc2n1-c1ccc(F)cc1] 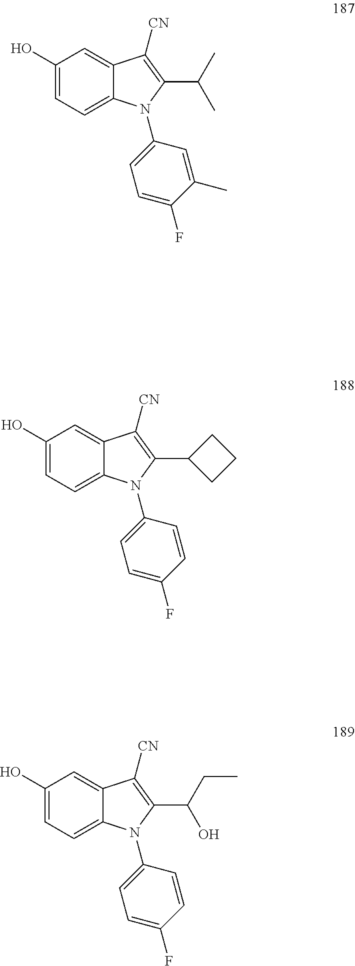 [SMILES: Cc1cc(-n2c(C(C)C)c(C#N)c3cc(O)ccc32)ccc1F ; N#Cc1c(C2CCC2)n(-c2ccc(F)cc2)c2ccc(O)cc12 ; CCC(O)c1c(C#N)c2cc(O)ccc2n1-c1ccc(F)cc1] 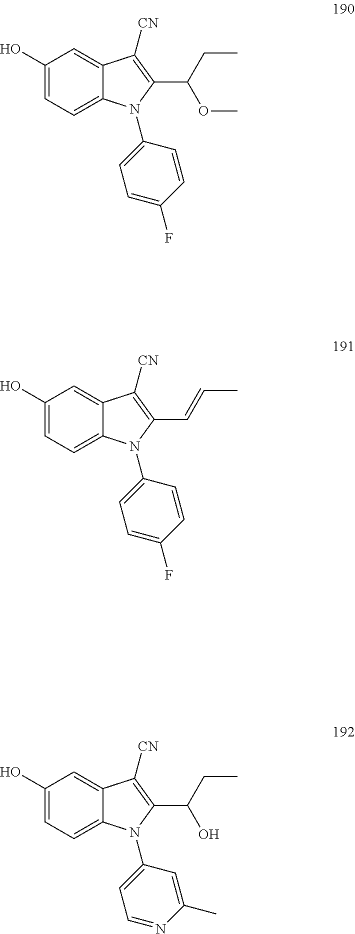 [SMILES: CCC(OC)c1c(C#N)c2cc(O)ccc2n1-c1ccc(F)cc1 ; C/C=C/c1c(C#N)c2cc(O)ccc2n1-c1ccc(F)cc1 ; CCC(O)c1c(C#N)c2cc(O)ccc2n1-c1ccnc(C)c1] 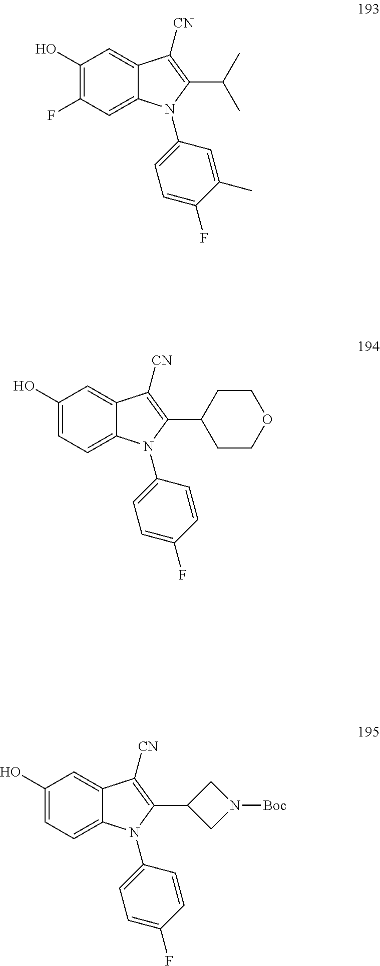 [SMILES: Cc1cc(-n2c(C(C)C)c(C#N)c3cc(O)c(F)cc32)ccc1F ; N#Cc1c(C2CCOCC2)n(-c2ccc(F)cc2)c2ccc(O)cc12 ; CC(C)(C)OC(=O)N1CC(c2c(C#N)c3cc(O)ccc3n2-c2ccc(F)cc2)C1] 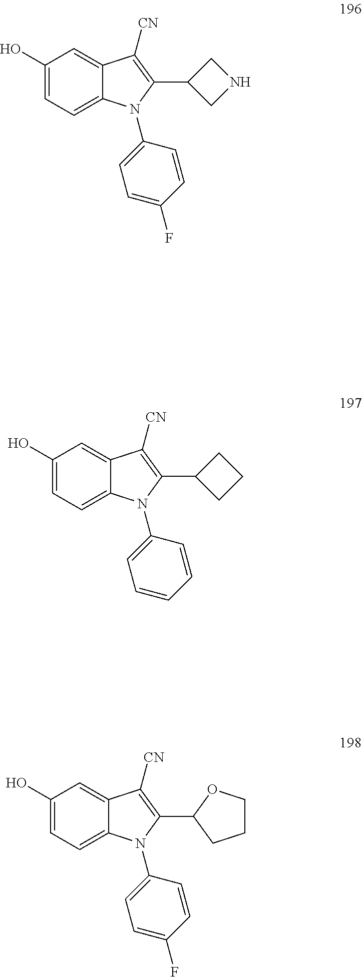 [SMILES: N#Cc1c(C2CNC2)n(-c2ccc(F)cc2)c2ccc(O)cc12 ; N#Cc1c(C2CCC2)n(-c2ccccc2)c2ccc(O)cc12 ; N#Cc1c(C2CCCO2)n(-c2ccc(F)cc2)c2ccc(O)cc12] 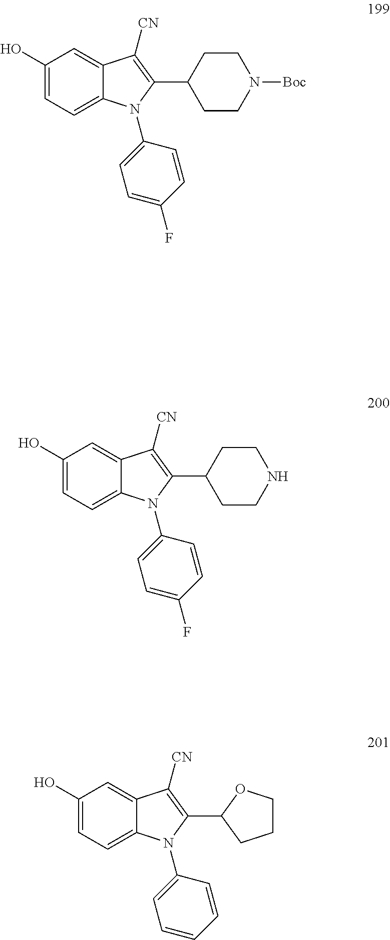 [SMILES: CC(C)(C)OC(=O)N1CCC(c2c(C#N)c3cc(O)ccc3n2-c2ccc(F)cc2)CC1 ; N#Cc1c(C2CCNCC2)n(-c2ccc(F)cc2)c2ccc(O)cc12 ; N#Cc1c(C2CCCO2)n(-c2ccccc2)c2ccc(O)cc12] 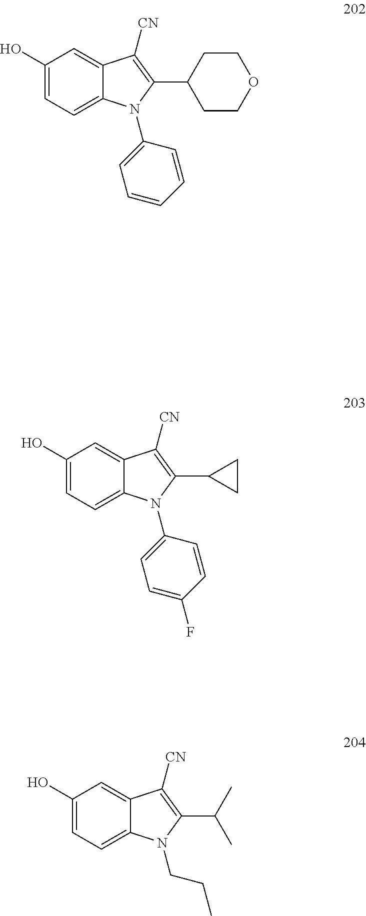 [SMILES: N#Cc1c(C2CCOCC2)n(-c2ccccc2)c2ccc(O)cc12 ; N#Cc1c(C2CC2)n(-c2ccc(F)cc2)c2ccc(O)cc12 ; CCCn1c(C(C)C)c(C#N)c2cc(O)ccc21] 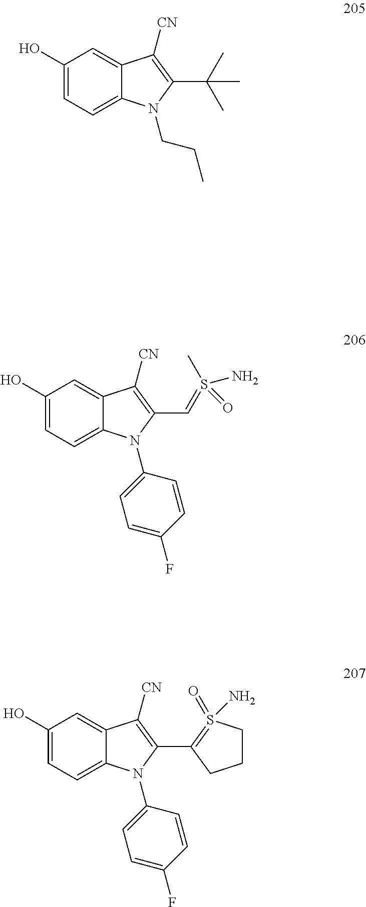 [SMILES: CCCn1c(C(C)(C)C)c(C#N)c2cc(O)ccc21 ; CS(N)(=O)=Cc1c(C#N)c2cc(O)ccc2n1-c1ccc(F)cc1 ; N#Cc1c(C2=S(N)(=O)CCC2)n(-c2ccc(F)cc2)c2ccc(O)cc12] 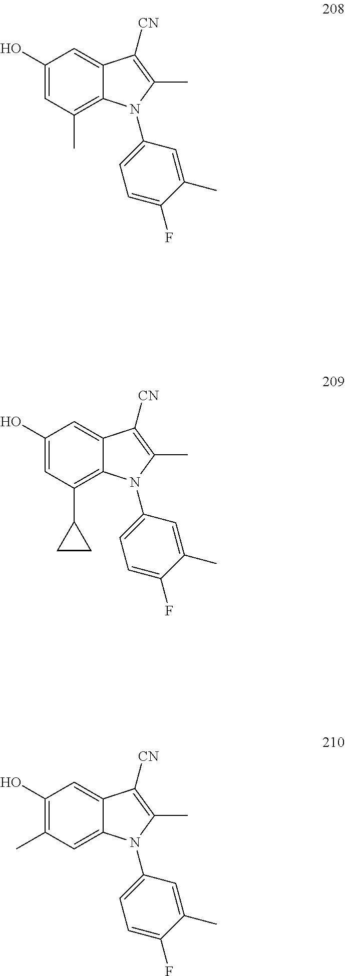 [SMILES: Cc1cc(-n2c(C)c(C#N)c3cc(O)cc(C)c32)ccc1F ; Cc1cc(-n2c(C)c(C#N)c3cc(O)cc(C4CC4)c32)ccc1F ; Cc1cc2c(cc1O)c(C#N)c(C)n2-c1ccc(F)c(C)c1] 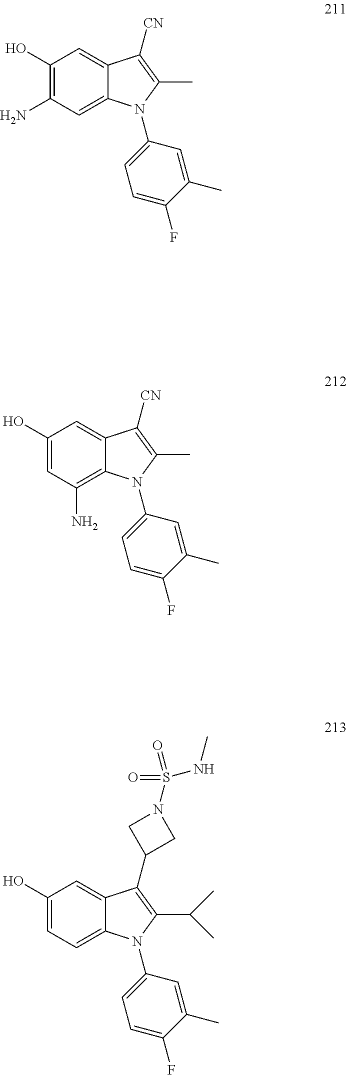 [SMILES: Cc1cc(-n2c(C)c(C#N)c3cc(O)c(N)cc32)ccc1F ; Cc1cc(-n2c(C)c(C#N)c3cc(O)cc(N)c32)ccc1F ; CNS(=O)(=O)N1CC(c2c(C(C)C)n(-c3ccc(F)c(C)c3)c3ccc(O)cc23)C1] 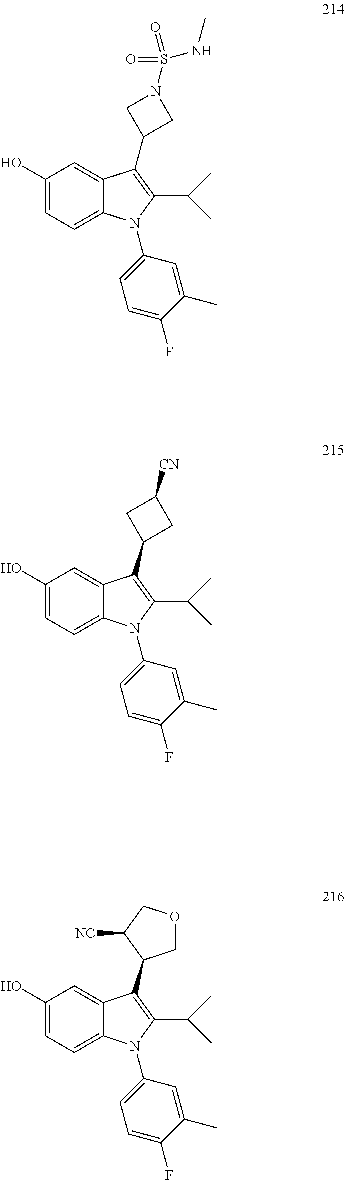 [SMILES: CNS(=O)(=O)N1CC(c2c(C(C)C)n(-c3ccc(F)c(C)c3)c3ccc(O)cc23)C1 ; Cc1cc(-n2c(C(C)C)c([C@H]3C[C@@H](C#N)C3)c3cc(O)ccc32)ccc1F ; Cc1cc(-n2c(C(C)C)c([C@@H]3COC[C@@H]3C#N)c3cc(O)ccc32)ccc1F] 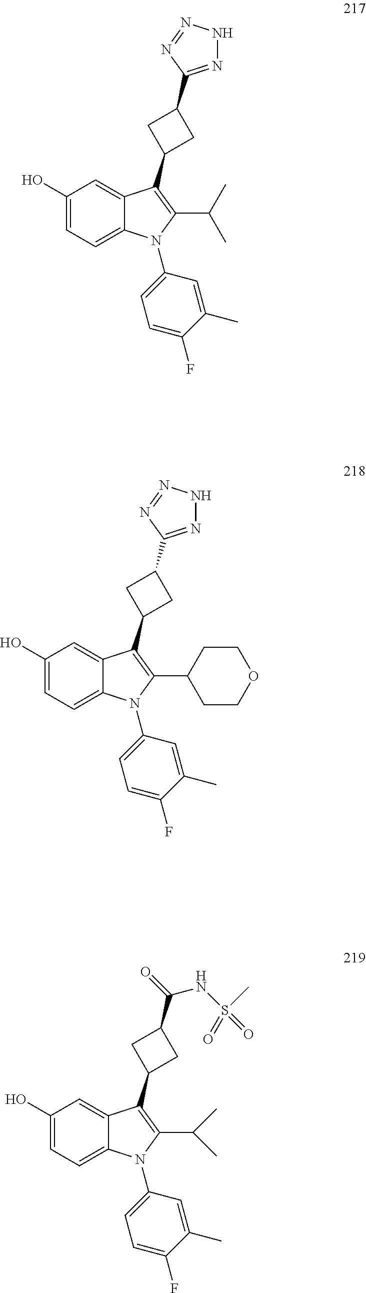 [SMILES: Cc1cc(-n2c(C(C)C)c([C@H]3C[C@@H](c4nn[nH]n4)C3)c3cc(O)ccc32)ccc1F ; Cc1cc(-n2c(C3CCOCC3)c([C@H]3C[C@H](c4nn[nH]n4)C3)c3cc(O)ccc32)ccc1F ; Cc1cc(-n2c(C(C)C)c([C@H]3C[C@@H](C(=O)NS(C)(=O)=O)C3)c3cc(O)ccc32)ccc1F] 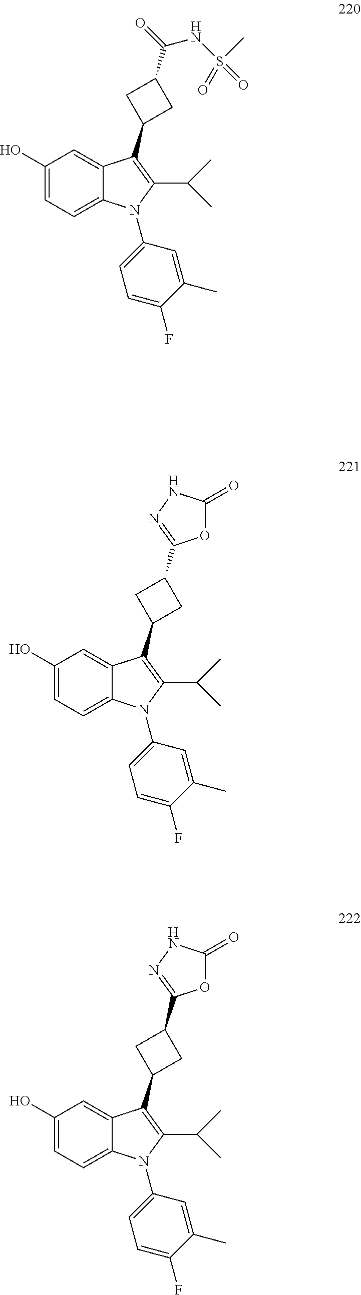 [SMILES: Cc1cc(-n2c(C(C)C)c([C@H]3C[C@H](C(=O)NS(C)(=O)=O)C3)c3cc(O)ccc32)ccc1F ; Cc1cc(-n2c(C(C)C)c([C@H]3C[C@H](c4n[nH]c(=O)o4)C3)c3cc(O)ccc32)ccc1F ; Cc1cc(-n2c(C(C)C)c([C@H]3C[C@@H](c4n[nH]c(=O)o4)C3)c3cc(O)ccc32)ccc1F] 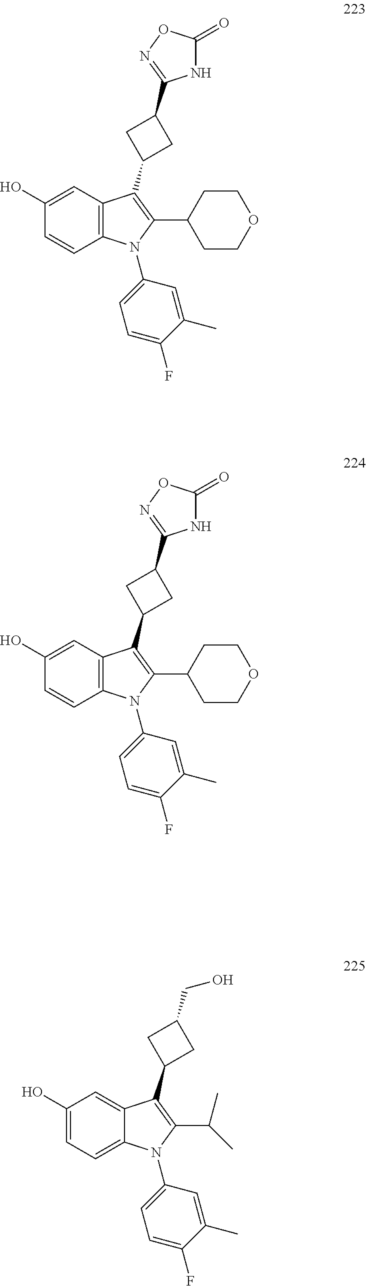 [SMILES: Cc1cc(-n2c(C3CCOCC3)c([C@H]3C[C@H](c4noc(=O)[nH]4)C3)c3cc(O)ccc32)ccc1F ; Cc1cc(-n2c(C3CCOCC3)c([C@H]3C[C@@H](c4noc(=O)[nH]4)C3)c3cc(O)ccc32)ccc1F ; Cc1cc(-n2c(C(C)C)c([C@H]3C[C@H](CO)C3)c3cc(O)ccc32)ccc1F] 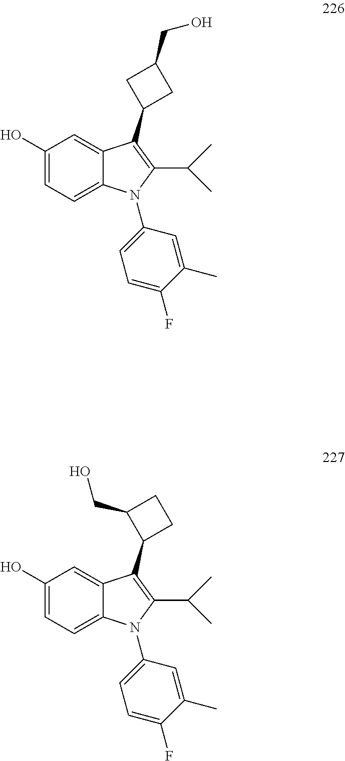 [SMILES: Cc1cc(-n2c(C(C)C)c([C@H]3C[C@@H](CO)C3)c3cc(O)ccc32)ccc1F ; Cc1cc(-n2c(C(C)C)c([C@@H]3CC[C@@H]3CO)c3cc(O)ccc32)ccc1F]
a tautomer thereof, a deuterated derivative of the compound or tautomer, or a pharmaceutically acceptable salt of any of the foregoing.

34. A pharmaceutical composition comprising at least one compound, tautomer, deuterated derivative, or pharmaceutically acceptable salt according to any one of claims 1 to 33.

35. A method of treating alpha-1 antitrypsin (AAT) deficiency comprising administering to a patient in need thereof a therapeutically effective amount of at least one compound, tautomer, deuterated derivative, or pharmaceutically acceptable salt according to any one of claims 1 to 33, or a therapeutically effective amount of a pharmaceutical composition according to claim 34.

36. A method of modulating alpha-1 antitrypsin (AAT) activity comprising the step of contacting said AAT with a therapeutically effective amount of at least one compound, tautomer, deuterated derivative, or pharmaceutically acceptable salt according to any one of claims 1 to 33, or a therapeutically effective amount of a pharmaceutical composition according to claim 34.

37. The method of claim 35 or claim 36, wherein said therapeutically effective amount of the at least one compound, tautomer, deuterated derivative, or pharmaceutically acceptable salt is administered in combination with AAT augmentation therapy and/or AAT replacement therapy.
